(19) United States
(12) Patent Application Publication
(10) Pub. No.: US 2004/0043930 A1
(43) Pub. Date: Mar. 4, 2004

(54) Novel proteins and nucleic acids encoding same

(76) Inventors: David W. Anderson, Plantsville, CT (US); Patricia Bento, Wolcott, CT (US); Ferenc Boldog, North Haven, CT (US); Catherine Burgess, Wethersfield, CT (US); Stacie Casman, North Haven, CT (US); Katarzyna Furtak, Ansonia, CT (US); Linda Gorman, Branford, CT (US); Bonnie Gould-Rothberg, Guilford, CT (US); Erik Gunther, Branford, CT (US); Melvyn Heyes, New Haven, CT (US); Li Li, Branford, CT (US); Kimberly Spytek, Ellington, CT (US); David Stone, Guilford, CT (US); Mei Zhong, Branford, CT (US); Uriel Malyankar, Branford, CT (US); Shlomit Edinger, New Haven, CT (US); Meera Patturajan, Branford, CT (US); Mark Rothenberg, Clinton, CT (US); Glennda Smithson, Guilford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/403,161

(22) Filed: Mar. 31, 2003

Related U.S. Application Data
(63) Continuation-in-part of application No. 09/779,679, filed on Feb. 8, 2001, now abandoned.
(60) Provisional application No. 60/370,349, filed on Apr. 5, 2002.
(60) Provisional application No. 60/384,543, filed on May 30, 2002.
(60) Provisional application No. 60/370,969, filed on Apr. 8, 2002.
(60) Provisional application No. 60/403,748, filed on Aug. 15, 2002.
(60) Provisional application No. 60/372,019, filed on Apr. 12, 2002.
(60) Provisional application No. 60/374,379, filed on Apr. 22, 2002.
(60) Provisional application No. 60/181,045, filed on Feb. 8, 2000.

(51) Int. Cl. A61K 38/17; C07K 14/47; C12P 21/02; C12N 5/06; C07H 21/04

(52) U.S. Cl. 514/12; 435/69.1; 435/320.1; 435/325; 530/350; 536/23.5

(57) ABSTRACT

The present invention provides novel isolated polynucleotides and small molecule target polypeptides encoded by the polynucleotides. Antibodies that immunospecifically bind to a novel small molecule target polypeptide or any derivative, variant, mutant or fragment of that polypeptide, polynucleotide or antibody are disclosed, as are methods in which the small molecule target polypeptide, polynucleotide and antibody are utilized in the detection and treatment of a broad range of pathological states. More specifically, the present invention discloses methods of using recombinantly expressed and/or endogenously expressed proteins in various screening procedures for the purpose of identifying therapeutic antibodies and therapeutic small molecules associated with diseases. The invention further discloses therapeutic, diagnostic and research methods for diagnosis, treatment, and prevention of disorders involving any one of these novel human nucleic acids and proteins.

### RELATED APPLICATIONS

This application claims priority to U.S. Ser. No. 60/370349, filed Apr. 5, 2002; U.S. Ser. No. 60/384543, filed May 30, 2002; U.S. Ser. No. 60/370969, filed Apr. 8, 2002; U.S. Ser. No. 60/403748, filed Aug. 15, 2002; U.S. Ser. No. 60/372019, filed Apr. 12, 2002; and U.S. Ser. No. 60/374379, filed Apr. 22, 2002, and this application is a continuation-in-part of U.S. Ser. No. 09/779679, filed Feb. 8, 2001; which claims priority to U.S. Ser. No. 60/181045, filed Feb. 8, 2000; U.S. Ser. No. 10/055877, filed Jan. 22, 2002; which claims priority to U.S. Ser. No. 60/262892, filed Jan. 19, 2001; U.S. Ser. No. 10/038854, filed Dec. 31, 2001; which claims priority to U.S. Ser. No. 60/259785, filed Jan. 4, 2001; and U.S. Ser. No. 60/294080, filed May 29, 2001; U.S. Ser. No. 10/044564, filed Jan. 11, 2002; which claims priority to U.S. Ser. No. 60/261026, filed Jan. 11, 2001; and U.S. Ser. No. 60/313170, filed Aug. 17, 2001; U.S. Ser. No. 10/092900, filed Mar. 7, 2002; which claims priority to U.S. Ser. No. 60/299027, filed Jun. 18, 2001; U.S. Ser. No. 10/115479, filed Apr. 2, 2002; which claims priority to U.S. Ser. No. 60/283657, filed Apr. 13, 2001; U.S. Ser. No. 10/160619, filed Jun. 3, 2002; which claims priority to U.S. Ser. No. 60/295607, filed Jun. 4, 2001; U.S. Ser. No. 10/287226, filed Nov. 4, 2002; which claims priority to U.S. Ser. No. 60/333461, filed Nov. 27, 2001; U.S. Ser. No. 10/307719, filed Dec. 2, 2002; which claims priority to U.S. Ser. No. 60/339286, filed Dec. 11, 2001; U.S. Ser. No. 10/093463, filed Mar. 8, 2002 which claims priority to U.S. Ser. No. 60/274191, filed Mar. 8, 2001; U.S. Ser. No. 60/287424, filed Apr. 30, 2001; and U.S. Ser. No. 10/161493, filed Jun. 3, 2002, which claims priority to U.S. Ser. No. 60/337524, filed Nov. 16, 2001 each of which is incorporated herein by reference in its entirety.

### FIELD OF THE INVENTION

The present invention relates to novel polypeptides that are targets of small molecule drugs and that have properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Small molecule targets have been implicated in various disease states or pathologies. These targets may be proteins, and particularly enzymatic proteins, which are acted upon by small molecule drugs for the purpose of altering target function and achieving a desired result. Cellular, animal and clinical studies can be performed to elucidate the genetic contribution to the etiology and pathogenesis of conditions in which small molecule targets are implicated in a variety of physiologic, pharmacologic or native states. These studies utilize the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the objective of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

### SUMMARY OF THE INVENTION

The invention includes nucleic acid sequences and the novel polypeptides they encode. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid, which represents the nucleotide sequence selected from the group consisting of SEQ ID NO: 2n-1, wherein n is an integer between 1 and 48, or polypeptide sequences, which represents the group consisting of SEQ ID NO: 2n, wherein n is an integer between 1 and 48.

In one aspect, the invention provides an isolated polypeptide comprising a mature form of a NOVX amino acid. One example is a variant of a mature form of a NOVX amino acid sequence, wherein any amino acid in the mature form is changed to a different amino acid, provided that no more than 15% of the amino acid residues in the sequence of the mature form are so changed. The amino acid can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of these. In another aspect, the invention also includes an isolated nucleic acid that encodes a NOVX polypeptide, or a fragment, homolog, analog or derivative thereof.

Also included in the invention is a NOVX polypeptide that is a naturally occurring allelic variant of a NOVX sequence. In one embodiment, the allelic variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution. In one embodiment, the invention discloses a method for determining the presence or amount of the NOVX polypeptide in a sample. The method involves the steps of: providing a sample; introducing the sample to an antibody that binds immunospecifically to the polypeptide; and determining the presence or amount of antibody bound to the NOVX polypeptide, thereby determining the presence or amount of the NOVX polypeptide in the sample. In another embodiment, the invention provides a method for determining the presence of or predisposition to a disease associated with altered levels of a NOVX polypeptide in a mammalian subject. This method involves the steps of: measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In a further embodiment, the invention includes a method of identifying an agent that binds to a NOVX polypeptide. This method involves the steps of: introducing the polypeptide to the agent; and determining whether the agent binds to the polypeptide. In various embodiments, the agent is a cellular receptor or a downstream effector.

In another aspect, the invention provides a method for identifying a potential therapeutic agent for use in treatment of a pathology, wherein the pathology is related to aberrant expression or aberrant physiological interactions of a NOVX polypeptide. The method involves the steps of: providing a cell expressing the NOVX polypeptide and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent. In another aspect, the invention describes a method for screening for a modulator of activity or of latency or predisposition to a pathology associated with the NOVX polypeptide. This method involves the following steps: administering a test compound to a test animal at increased risk for a pathology associated with the NOVX polypeptide, wherein the test animal recombinantly expresses the NOVX polypeptide. This method involves the steps of measuring the activity of the NOVX polypeptide in the test animal after administering the compound of step; and comparing the activity of the protein in the test animal with the activity of the NOVX polypeptide in a control animal not administered the polypeptide, wherein a change in the activity of the NOVX polypeptide in the test animal relative to the control animal indicates the test compound is a modulator of latency of, or predisposition to, a pathology associated with the NOVX polypeptide. In one embodiment, the test animal is a recombinant test animal that expresses a test protein transgene or expresses the transgene under the control of a promoter at an increased level relative to a wild-type test animal, and wherein the promoter is not the native gene promoter of the transgene. In another aspect, the invention includes a method for modulating the activity of the NOVX polypeptide, the method comprising introducing a cell sample expressing the NOVX polypeptide with a compound that binds to the polypeptide in an amount sufficient to modulate the activity of the polypeptide.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n-1, wherein n is an integer between 1 and 48, or a complement of the nucleotide sequence. In another aspect, the invention provides a vector or a cell expressing a NOVX nucleotide sequence.

In one embodiment, the invention discloses a method for modulating the activity of a NOVX polypeptide. The method includes the steps of: introducing a cell sample expressing the NOVX polypeptide with a compound that binds to the polypeptide in an amount sufficient to modulate the activity of the polypeptide. In another embodiment, the invention includes an isolated NOVX nucleic acid molecule comprising a nucleic acid sequence encoding a polypeptide comprising a NOVX amino acid sequence or a variant of a mature form of the NOVX amino acid sequence, wherein any amino acid in the mature form of the chosen sequence is changed to a different amino acid, provided that no more than 15% of the amino acid residues in the sequence of the mature form are so changed. In another embodiment, the invention includes an amino acid sequence that is a variant of the NOVX amino acid sequence, in which any amino acid specified in the chosen sequence is changed to a different amino acid, provided that no more than 15% of the amino acid residues in the sequence are so changed.

In one embodiment, the invention discloses a NOVX nucleic acid fragment encoding at least a portion of a NOVX polypeptide or any variant of the polypeptide, wherein any amino acid of the chosen sequence is changed to a different amino acid, provided that no more than 10% of the amino acid residues in the sequence are so changed. In another embodiment, the invention includes the complement of any of the NOVX nucleic acid molecules or a naturally occurring allelic nucleic acid variant. In another embodiment, the invention discloses a NOVX nucleic acid molecule that encodes a variant polypeptide, wherein the variant polypeptide has the polypeptide sequence of a naturally occurring polypeptide variant. In another embodiment, the invention discloses a NOVX nucleic acid, wherein the nucleic acid molecule differs by a single nucleotide from a NOVX nucleic acid sequence.

In another aspect, the invention includes a NOVX nucleic acid, wherein one or more nucleotides in the NOVX nucleotide sequence is changed to a different nucleotide provided that no more than 15% of the nucleotides are so changed. In one embodiment, the invention discloses a nucleic acid fragment of the NOVX nucleotide sequence and a nucleic acid fragment wherein one or more nucleotides in the NOVX nucleotide sequence is changed from that selected from the group consisting of the chosen sequence to a different nucleotide provided that no more than 15% of the nucleotides are so changed. In another embodiment, the invention includes a nucleic acid molecule wherein the nucleic acid molecule hybridizes under stringent conditions to a NOVX nucleotide sequence or a complement of the NOVX nucleotide sequence. In one embodiment, the invention includes a nucleic acid molecule, wherein the sequence is changed such that no more than 15% of the nucleotides in the coding sequence differ from the NOVX nucleotide sequence or a fragment thereof.

In a further aspect, the invention includes a method for determining the presence or amount of the NOVX nucleic acid in a sample. The method involves the steps of: providing the sample; introducing the sample to a probe that binds to the nucleic acid molecule; and determining the presence or amount of the probe bound to the NOVX nucleic acid molecule, thereby determining the presence or amount of the NOVX nucleic acid molecule in the sample. In one embodiment, the presence or amount of the nucleic acid molecule is used as a marker for cell or tissue type.

In another aspect, the invention discloses a method for determining the presence of or predisposition to a disease associated with altered levels of the NOVX nucleic acid molecule of in a first mammalian subject. The method involves the steps of: measuring the amount of NOVX nucleic acid in a sample from the first mammalian subject; and comparing the amount of the nucleic acid in the sample of step (a) to the amount of NOVX nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Requences and Corresponding SEQ ID Numbers

| | | SEQ ID | SEQ ID | |
|---|---|---|---|---|
| | | NO | NO | |
| NOVX | Internal | (nucleic | (amino | |
| Assignment | Identification | acid) | acid) | Homology |
| NOV1a | CG164221-01 | 1 | 2 | Sequence 1 from Patent WO02074987 - |
| | | | | Homo sapiens |
| NOV1b | CG164221-02 | 3 | 4 | Sequence 1 from Patent WO02074987 - |
| | | | | Homo sapiens |
| NOV2a | CG180777-01 | 5 | 6 | Cell cycle related kinase - Homo |
| | | | | sapiens |
| NOV2b | CG180777-02 | 7 | 8 | Cell cycle related kinase - Homo |
| | | | | sapiens |
| NOV3a | CG181825-01 | 9 | 10 | Aquaporin 6 (Aquaporin-2 like) (hKID) - |
| | | | | Homo sapiens |
| NOV4a | CG50183-01 | 11 | 12 | C—C chemokine receptor type 11 (C—C |
| | | | | CKR-11) (CC-CKR-11) (CCR-11) |
| | | | | (Chemokine receptor-like 1) (CCRL1) |
| | | | | (CCX CKR) - Homo sapiens |
| NOV5a | CG50249-01 | 13 | 14 | Voltage gated potassium channel |
| | | | | Kv3.2b (Potassium voltage-gated |
| | | | | potassium channel subfamily C |
| | | | | member 2) - Homo sapiens |
| NOV5b | CG50249-02 | 15 | 16 | Voltage gated potassium channel |
| | | | | Kv3.2b (Potassium voltage-gated |
| | | | | potassium channel subfamily C |
| | | | | member 2) - Homo sapiens |
| NOV5c | CG50249-03 | 17 | 18 | Voltage gated potassium channel |
| | | | | Kv3.2b (Potassium voltage-gated |
| | | | | potassium channel subfamily C |
| | | | | member 2) - Homo sapiens |
| NOV5d | CG50249-04 | 19 | 20 | Voltage gated potassium channel |
| | | | | Kv3.2b (Potassium voltage-gated |
| | | | | potassium channel subfamily C |
| | | | | member 2) - Homo sapiens |
| NOV6a | CG54236-02 | 21 | 22 | Cysteinyl leukotriene receptor 2 |
| | | | | (CysLTR2) (PSEC0146) (HG57) |
| | | | | (HPN321) (hGPCR21) - Homo sapiens |
| NOV6b | CG54236-01 | 23 | 24 | Cysteinyl leukotriene receptor 2 |
| | | | | (CysLTR2) (PSEC0146) (HG57) |
| | | | | (HPN321) (hGPCR21) - Homo sapiens |
| NOV7a | CG54566-01 | 25 | 26 | Sequence 11 from Patent WO0194416 - |
| | | | | Homo sapiens |
| NOV8a | CG55912-01 | 27 | 28 | Voltage-dependent calcium channel |
| | | | | gamma-8 subunit (Neuronal voltage- |
| | | | | gated calcium channel gamma-8 |
| | | | | subunit) - Rattus norvegicus |
| NOV9a | CG56001-01 | 29 | 30 | D-beta-hydroxybutyrate |
| | | | | dehydrogenase, mitochondrial |
| | | | | precursor (EC 1.1.1.30) (BDH) (3- |
| | | | | hydroxybutyrate dehydrogenase) - |
| | | | | Homo sapiens |
| NOV9b | CG56001-02 | 31 | 32 | D-beta-hydroxybutyrate |
| | | | | dehydrogenase, mitochondrial |
| | | | | precursor (EC 1.1.1.30) (BDH) (3- |
| | | | | hydroxybutyrate dehydrogenase) - |
| | | | | Homo sapiens |
| NOV10a | CG56151-01 | 33 | 34 | Solute carrier family 2, facilitated |
| | | | | glucose transporter, member 2 (Glucose |
| | | | | transporter type 2, liver) - Homo |
| | | | | sapiens |
| NOV10b | 246837923 | 35 | 36 | Solute carrier family 2, facilitated |
| | | | | glucose transporter, member 2 (Glucose |
| | | | | transporter type 2, liver) - Homo |
| | | | | sapiens |
| NOV10c | 246837941 | 37 | 38 | Solute carrier family 2, facilitated |
| | | | | glucose transporter, member 2 (Glucose |
| | | | | transporter type 2, liver) - Homo |
| | | | | sapiens |
| NOV10d | CG56151-02 | 39 | 40 | Solute carrier family 2, facilitated |
| | | | | glucose transporter, member 2 (Glucose |
| | | | | transporter type 2, liver) - Homo |
| | | | | sapiens |
| NOV10e | CG56151-03 | 41 | 42 | Solute carrier family 2, facilitated |
| | | | | glucose transporter, member 2 (Glucose |
| | | | | transporter type 2, liver) - Homo |
| | | | | sapiens |
| NOV10f | CG56151-04 | 43 | 44 | Solute carrier family 2, facilitated |
| | | | | glucose transporter, member 2 (Glucose |
| | | | | transporter type 2, liver) - Homo |
| | | | | sapiens |
| NOV11a | CG56155-02 | 45 | 46 | Plasma kallikrein precursor (EC |
| | | | | 3.4.21.34) (Plasma prekallikrein) |
| | | | | (Kininogenin) (Fletcher factor) - Homo |
| | | | | sapiens |
| NOV11b | 227803167 | 47 | 48 | Plasma kallikrein precursor (EC |
| | | | | 3.4.21.34) (Plasma prekallikrein) |
| | | | | (Kininogenin) (Fletcher factor) - Homo |
| | | | | sapiens |
| NOV11c | CG56155-01 | 49 | 50 | Plasma kallikrein precursor (EC |
| | | | | 3.4.21.34) (Plasma prekallikrein) |
| | | | | (Kininogenin) (Fletcher factor) - Homo |
| | | | | sapiens |
| NOV11d | CG56155-03 | 51 | 52 | Plasma kallikrein precursor (EC |
| | | | | 3.4.21.34) (Plasma prekallikrein) |
| | | | | (Kininogenin) (Fletcher factor) - Homo |
| | | | | sapiens |
| NOV12a | CG56262-01 | 53 | 54 | Putative calcium binding transporter - |
| | | | | Homo sapiens |
| NOV13a | CG56829-01 | 55 | 56 | Testis-specific serine/threonine kinase |
| | | | | 3 (Similar to serine/threonine kinase) |
| | | | | (TSSK3) - Homo sapiens |
| NOV14a | CG57183-01 | 57 | 58 | Fibroblast growth factor receptor 3 |
| | | | | precursor (EC 2.7.1.112) (FGFR-3) - |
| | | | | Homo sapiens |
| NOV14b | CG57183-02 | 59 | 60 | Fibroblast growth factor receptor 3 |
| | | | | precursor (EC 2.7.1.112) (FGFR-3) - |
| | | | | Homo sapiens |
| NOV15a | CG57341-01 | 61 | 62 | Sequence 2 from Patent WO0144446 - |
| | | | | Homo sapiens |
| NOV16a | CG57460-01 | 63 | 64 | Hypothetical protein FLJ37478 - Homo |
| | | | | sapiens |
| NOV17a | CG57570-01 | 65 | 66 | Solute carrier family 41 member 1 - |
| | | | | Homo sapiens |
| NOV18a | CG57758-02 | 67 | 68 | Na+ coupled citrate transporter protein - |
| | | | | Homo sapiens |
| NOV18b | CG57758-01 | 69 | 70 | Na+ coupled citrate transporter protein - |
| | | | | Homo sapiens |
| NOV18c | CG57758-03 | 71 | 72 | Na+ coupled citrate transporter protein - |
| | | | | Homo sapiens |
| NOV18d | CG57758-04 | 73 | 74 | Na+ coupled citrate transporter protein - |
| | | | | Homo sapiens |
| NOV18e | CG57758-05 | 75 | 76 | Na+ coupled citrate transporter protein - |
| | | | | Homo sapiens |
| NOV19a | CG59693-01 | 77 | 78 | Aldo-keto reductase family 1 member |
| | | | | C1 (EC 1.1.1.-) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV19b | CG59693-02 | 79 | 80 | Aldo-keto reductase family 1 member |
| | | | | C1 (EC 1.1.1.-) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV19c | CG59693-03 | 81 | 82 | Aldo-keto reductase family 1 member |
| | | | | C1 (EC 1.1.1.-) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV19d | CG59693-04 | 83 | 84 | Aldo-keto reductase family 1 member |
| | | | | C1 (EC 1.1.1.-) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV19e | CG59693-05 | 85 | 86 | Aldo-keto reductase family 1 member |
| | | | | C1 (EC 1.1.1.-) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV19f | CG59693-06 | 87 | 88 | Aldo-keto reductase family 1 member |
| | | | | C1 (EC 1.1.1.-) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV19g | CG59693-07 | 89 | 90 | Aldo-keto reductase family 1 member |
| | | | | C1 (EC 1.1.1.-) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV19h | CG59693-08 | 91 | 92 | Aldo-keto reductase family 1 member |
| | | | | C1 (EC 1.1.1.-) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV19i | CG59693-09 | 93 | 94 | Aldo-keto reductase family 1 member |
| | | | | C1 (EC 1.1.1.-) (Trans-1,2- |
| | | | | dihydrobenzene-1,2-diol |
| | | | | dehydrogenase) (EC 1.3.1.20) (High- |
| | | | | affinity hepatic bile acid-binding |
| | | | | protein) (HBAB) (Chlordecone |
| | | | | reductase homolog HAKRC) |
| | | | | (Dihydrodiol dehydrogenase 2) (DD2) |
| | | | | (20 alpha-hydroxysteroid |
| | | | | dehydrogenase) - Homo sapiens |
| NOV20a | CG93088-01 | 95 | 96 | Adult male lung cDNA, RIKEN full- |
| | | | | length enriched library, |
| | | | | clone: 1200003C15 |
| | | | | product: hypothetical protein, full insert |
| | | | | sequence - Mus musculus |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, the metabolic syndrome X and wasting disorders associated with chronic diseases and various cancers, as well as conditions such as transplantation and fertility.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g., detection of a variety of cancers. SNP analysis for each NOVX, if applicable, is presented in Example D.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 48; (b) a variant of a mature form of the amino acid sequence selected from the group consisting of SEQ ID NO: 2n, wherein n is an integer between 1 and 48, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 48; (d) a variant of the amino acid sequence selected from the group consisting of SEQ ID NO: 2n, wherein n is an integer between 1 and 48 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 48; (b) a variant of a mature form of the amino acid sequence selected from the group consisting of SEQ ID NO: 2n, wherein n is an integer between 1 and 48 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 48; (d) a variant of the amino acid sequence selected from the group consisting of SEQ ID NO: 2n, wherein n is an integer between 1 and 48, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 48 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n-1, wherein n is an integer between 1 and 48; (b) a nucleotide sequence wherein one or more nucleotides in the nucleotide sequence selected from the group consisting of SEQ ID NO: 2n-1, wherein n is an integer between 1 and 48 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n-1, wherein n is an integer between 1 and 48; and (d) a nucleic acid fragment wherein one or more nucleotides in the nucleotide sequence selected from the group consisting of SEQ ID NO: 2n-1, wherein n is an integer between 1 and 48 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO: 2n-1, wherein n is an integer between 1 and 48, or a complement of this nucleotide sequence, can be isolated using standard molecular biology techniques and the sequence information provided herein. Using all or a portion of the nucleic acid sequence of SEQ ID NO: 2n-1, wherein n is an integer between 1 and 48, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, NY, 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO: 2n-1, wherein n is an integer between 1 and 48, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO: 2n-1, wherein n is an integer between 1 and 48, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO: 2n-1, wherein n is an integer between 1 and 48, is one that is sufficiently complementary to the nucleotide sequence of SEQ ID NO: 2n-1, wherein n is an integer between 1 and 48, that it can hydrogen bond with few or no mismatches to the nucleotide sequence shown in SEQ ID NO: 2n-1, wherein n is an integer between 1 and 48, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g., they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g., Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO: 2n-1, wherein n is an integer between 1 and 48, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g., from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO: 2n-1, wherein n is an integer between 1 and 48; or an anti-sense strand nucleotide sequence of SEQ ID NO: 2n-1, wherein n is an integer between 1 and 48; or of a naturally occurring mutant of SEQ ID NO: 2n-1, wherein n is an integer between 1 and 48.

Probes based on the human NOVX nucleotide sequences can be used to detect transcripts or genomic sequences encoding the same or homologous proteins. In various embodiments, the probe has a detectable label attached, e.g., the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO: 2n-1, wherein n is an integer between 1 and 48, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO: 2n-1, wherein n is an integer between 1 and 48, due to degeneracy of the genetic code and thus encode the same NOVX proteins as that encoded by the nucleotide sequences of SEQ ID NO: 2n-1, wherein n is an integer between 1 and 48. In another embodiment, an isolated nucleic acid molecule of the invention has a nucleotide sequence encoding a protein having an amino acid sequence of SEQ ID NO: 2n, wherein n is an integer between 1 and 48.

In addition to the human NOVX nucleotide sequences of SEQ ID NO: 2n-1, wherein n is an integer between 1 and 48, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO: 2n-1, wherein n is an integer between 1 and 48, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 2n-1, wherein n is an integer between 1 and 48. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO: 2n-1, wherein n is an integer between 1 and 48, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 2n-1, wherein n is an integer between 1 and 48, or fragments, analogs or derivatives thereof, under conditions of moderate stringency is provided. A non-limiting example of moderate stringency hybridization conditions are hybridization in 6×SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, N.Y.

In a third embodiment, a nucleic acid that is hybridizable to the nucleic acid molecule comprising the nucleotide sequences of SEQ ID NO: 2n-1, wherein n is an integer between 1 and 48, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4),5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO: 2n-1, wherein n is an integer between 1 and 48, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO: 2n, wherein n is an integer between 1 and 48. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are not particularly amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO: 2n-1, wherein n is an integer between 1 and 48, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO: 2n, wherein n is an integer between 1 and 48. Preferably, the protein encoded by the nucleic acid molecule is at least about 60% homologous to SEQ ID NO: 2n, wherein n is an integer between 1 and 48; more preferably at least about 70% homologous to SEQ ID NO: 2n, wherein n is an integer between 1 and 48; still more preferably at least about 80% homologous to SEQ ID NO: 2n, wherein n is an integer between 1 and 48; even more preferably at least about 90% homologous to SEQ ID NO: 2n, wherein n is an integer between 1 and 48; and most preferably at least about 95% homologous to SEQ ID NO: 2n, wherein n is an integer between 1 and 48.

An isolated nucleic acid molecule encoding a NOVX protein homologous to the protein of SEQ ID NO: 2n, wherein n is an integer between 1 and 48, can be created by introducing one or more nucleotide substitutions, additions or deletions into the nucleotide sequence of SEQ ID NO: 2n-1, wherein n is an integer between 1 and 48, such that one or more amino acid substitutions, additions or deletions are introduced into the encoded protein.

Mutations can be introduced any one of SEQ ID NO: 2n-1, wherein n is an integer between 1 and 48, by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO: 2n-1, wherein n is an integer between 1 and 48, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g., avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO62/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g., inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g., commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2×-proteinase-K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in-vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO: 2n-1, wherein n is an integer between 1 and 48, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO: 2n, wherein n is an integer between 1 and 48, or antisense nucleic acids complementary to a NOVX nucleic acid sequence of SEQ ID NO: 2n-1, wherein n is an integer between 1 and 48, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences that flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO: 2n-1, wherein n is an integer between 1 and 48). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)-amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization-triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO: 2n, wherein n is an integer between 1 and 48. The invention also includes a mutant or variant protein any of whose residues may be changed from the corresponding residues shown in any one of SEQ ID NO: 2n, wherein n is an integer between 1 and 48, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO: 2n, wherein n is an integer between 1 and 48) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO: 2n, wherein n is an integer between 1 and 48. In other embodiments, the NOVX protein is substantially homologous to SEQ ID NO: 2n, wherein n is an integer between 1 and 48, and retains the functional activity of the protein of SEQ ID NO: 2n, wherein n is an integer between 1 and 48, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO: 2n, wherein n is an integer between 1 and 48, and retains the functional activity of the NOVX proteins of SEQ ID NO: 2n, wherein n is an integer between 1 and 48.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO: 2n-1, wherein n is an integer between 1 and 48.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO: 2n, wherein n is an integer between 1 and 48, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g., promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, F_{ab}, F_{ab}, and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO: 2n, wherein n is an integer between 1 and 48, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen that is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse mycloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subdlones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals. For example, mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers that are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies can be prepared as full length antibodies or antibody fragments (e.g., F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective inmmobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fe receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fe region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fe regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i. e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidinibiotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand that may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, NJ, 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, useful expression vectors in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharnacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kudan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Baneji, et al., 1983. Cell 33: 729-740; Queen and Baltimore, 1983. Cell 33: 741-748), neuron-specific promoters (e.g., the neurofilament promoter; Byrne and Ruddle, 1989 Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO: 2n-1, wherein n is an integer between 1 and 48, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO: 2n-1, wherein n is an integer between 1 and 48), but more preferably, is a non-human homologue of a human NOVX gene. For example, a mouse homologue of human NOVX gene of SEQ ID NO: 2n-1, wherein n is an integer between 1 and 48, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g. a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et a., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds that bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g., a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e., intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g., stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Tritono® X-100, Tritono® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO: 2n-1, wherein n is an integer between 1 and 48, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO: 2n-1, wherein n is an integer between 1 and 48, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO: 2n-1, wherein n is an integer between 1 and 48, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7:5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265:12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

### Polynucleotide and Polypeptide Sequences, and Homology Data

### Example 1

The NOV1 clone was analyzed, and the nucleotide and encoded polypeptide sequences are shown in Table 1A.

TABLE 1A
NOV1 Sequence Analysis

| | | |
|---|---|---|
| NOV1a, CG164221-01 | SEQ ID NO: 1 | 2343 bp |
| DNA Sequence | ORF Start: ATG at 36 | ORF Stop: end of sequence |

GAGCGCTAACGTCTTTCTGTCTCCCCGCGGTGGTGATGACGGTGAAAACTGAGGCTGCTAAGGGCACC
CTCACTTACTCCAGGATCAGGGGCATGGTGGCAATTCTCATCGCTTTCATGAAGCAGAGGAGGATGGG
TCTGAACGACTTTATTCAGAAGATTGCCAATAACTCCTATGCATGCAAACACCCTGAAGTTCAGTCCA
TCTTGAAGATCTCCCAACCTCAGGAGCCTGAGCTTATGAATGCCAACCCTTCTCCTCCACCAAGTCCT
TCTCAGCAAATCAACCTTGGCCCGTCGTCCAATCCTCATGCTAAACCATCTGACTTTCACTTCTTGAA
AGTGATCGGAAAGGGCAGTTTTGGAAAGGTTCTTCTAGCAAGACACAAGGCAGAAGAAGTGTTCTATG
CAGTCAAAGTTTTACAGAAGAAAGCAATCCTGAAAAAGAAAGAGGAGAAGCATATTATGTCGGAGCGG
AATGTTCTGTTGAAGAATGTGAAGCACCCTTTCCTGGTGGGCCTTCACTTCTCTTTCCAGACTGCTGA
CAAATTGTACTTTGTCCTAGACTACATTAATGGTGGAGAGTTGTTCTACCATCTCCAGAGGGAACGCT
GCTTCCTGGAACCACGGGCTCGTTTCTATGCTGCTGAAATAGCCAGTGCCTTGGGCTACCTGCATTCA
CTGAACATCGTTTATAGAGACTTAAAACCAGAGAATATTTTGCTAGATTCACAGGGACACATTGTCCT
TACTGACTTCGGACTCTGCAAGGAGAACATTGAACACAACAGCACAACATCCACCTTCTGTGGCACGC
CGGAGTATCTCGCACCTGAGGTGCTTCATAAGCAGCCTTATGACAGGACTGTGGACTGGTGGTGCCTG
GGAGCTGTCTTGTATGAGATGCTGTATGGCCTGCCGCCTTTTTATAGCCGAAACACAGCTGAAATGTA
CGACAACATTCTGAACAAGCCTCTCCAGCTGAAACCAAATATTACAAATTCCGCAAGACACCTCCTGG
AGGGCCTCCTGCAGAAGGACAGGACAAAGCGGCTCGGGGCCAAGGATGACTTCATGGAGATTAAGAGT
CATGTCTTCTTCTCCTTAATTAACTGGGATGATCTCATTAATAAGAAGATTACTCCCCCTTTTAACCC
AAATGTGAGTGGGCCCAACGACCTACGGCACTTTGACCCCGAGTTTACCGAAGAGCCTGTCCCCAACT
CCATTGGCAAGTCCCCTGACAGCGTCCTCGTCACAGCCAGCGTCAAGGAAGCTGCCGAGGCTTTCCTA
GGCTTTTCCTATGCGCCTCCCACGGACTCTTTCCTCTGAACCCTGTTAGGGCTTGGTTTTAAAGGATT
TTATGTGTGTTTCCGAATGTTTTAGTTAGCCTTTTGGTGGAGCCGCCAGCTGACAGGACATCTTACAA
GAGAATTTGCACATCTCTGGAAGCTTAGCAATCTTATTGCACACTGTTCGCTGGAAGCTTTTTGAAGA
GCACATTCTCCTCAGTGAGCTCATGAGGTTTTCATTTTTATTCTTCCTTCCAACGTGGTGCTATCTCT
GAAACGAGCGTTAGAGTGCCGCCTTAGACGGAGGCAGGAGTTTCGTTAGAAAGCGGACGCTGTTCTAA
AAAAGGTCTCCTGCAGATCTGTCTGGGCTGTGATGACGAATATTATGAAATGTGCCTTTTCTGAAGAG
ATTGTGTTAGCTCCAAAGCTTTTCCTATCGCAGTGTTTCAGTTCTTTATTTTCCCTTGTGGATATGCT
GTGTGAACCGTCGTGTGAGTGTGGTATGCCTGATCACAGATGGATTTTGTTATAAGCATCAATGTGAC
ACTTGCAGGACACTACAACGTGGGACATTGTTTGTTTCTTCCATATTTGGAAGATAAATTTATGTGTA
GACTTTTTTGTAAGATACGGTTAATAACTAAAATTTATTGAAATGGTCTTGCAATGACTCGTATTCAG
ATGCTTAAAGAAAGCATTGCTGCTACAAATATTTCTATTTTTAGAAAGGGTTTTTATGGACCAATGCC
CCAGTTGTCAGTCAGAGCCGTTGGTGTTTTTCATTGTTTAAAATGTCACCTGTAAAATGGGCATTATT
TATGTTTTTTTTTTTGCATTCCTGATAATTGTATGTATTGTATAAAGAACGTCTGTACATTGGGTTAT
AACACTAGTATATTTAAACTTACAGGCTTATTTGTAATGTAAACCACCATTTTAATGTACTGTAATTA
ACATGGTTATAATACGTACAATCCTTCCCTCATCCCATCACACAACTTTTTTTGTGTGTGATAAACTG
ATTTTGGTTTGCAATAAAACCTTGAAAAATA

| | | | |
|---|---|---|---|
| NOV1a, CG164221-01 | | | |
| Protein Sequence | SEQ ID NO: 2 | 431 aa | MW at 48941.8 kD |

MTVKTEAAKGTLTYSRMRGMVAILIAFMKQRRMGLNDFIQKIANNSYACKHPEVQSILKISQPQEPEL
MNANPSPPPSPSQQINLGPSSNPHAKPSDFHFLKVIGKGSFGKVLLARHKAEEVFYAVKVLQKKAILK
KKEEKHIMSERNVLLKNVKHPFLVGLHFSFQTADKLYFVLDYINGGELFYHLQRERCFLEPRARFYAA
EIASALGYLHSLNIVYRDLKPENILLDSQGHIVLTDFGLCKENIEHNSTTSTFCGTPEYLAPEVLHKQ
PYDRTVDWWCLGAVLYEMLYGLPPFYSRNTAEMYDNILNKPLQLKPNITNSARHLLEGLLQKDRTKRL
GAKDDFMEIKSHVFFSLINWDDLINKKITPPFNPNVSGPNDLRHFDPEFTEEPVPNSIGKSPDSVLVT
ASVKEAAEAFLGFSYAPPTDSFL

| | | |
|---|---|---|
| NOV1b, CG164221-02 | SEQ ID NO: 3 | 1315 bp |
| DNA Sequence | ORF Start: ATG at 14 | ORF Stop: end of sequence |

CACCGGATCCACCATGACGGTGAAAACTGAGGCTGCTAAGGGCACCCTCACTTACTCCAGGATGAGGG
GCATGGTGGCAATTCTCATCGCTTTCATGAAGCAGAGGAGGATGGGTCTGAACGACTTTATTCAGAAG
ATTGCCAATAACTCCTATGCATGCAAACACCCTGAAGTTCAGTCCATCTTGAAGATCTCCCAACCTCA
GGAGCCTGAGCTTATGAATGCCAACCCTTCTCCTCCACCAAGTCCTTCTCAGCAAATCAACCTTGGCC
CGTCGTCCAATCCTCATGCTAAACCATCTGACTTTCACTTCTTGAAAGTGATCGGAAAGGGCAGTTTT
GGAAAGGTTCTTCTAGCAAGACACAAGGCAGAAGAAGTGTTCTATGCAGTCAAAGTTTTACAGAAGAA
AGCAATCCTGAAAAAGAAAGAGGAGAAGCATATTATGTCGGAGCGGAATGTTCTGTTGAAGAATGTGA
AGCACCCTTTCCTGGTGGGCCTTCACTTCTCTTTCCAGACTGCTGACAAATTGTACTTTGTCCTAGAC
TACATTAATGGTGGAGAGTTGTTCTACCATCTCCAGAGGGAACGCTGCTTCCTGGAACCACGGGCTCG
TTTCTATGCTGCTGAAATAGCCAGTGCCTTGGGCTACCTGCATTCACTGAACATCGTTTATAGAGACT
TAAAACCAGAGAATATTTTGCTAGATTCACAGGGACACATTGTCCTTACTGACTTCGGACTCTGCAAG
GAGAACATTGAACACAACAGCACAACATCCACCTTCTGTGGCACGCCGGAGTATCTCGCACCTGAGGT
GCTTCATAAGCAGCCTTATGACAGGACTGTGGACTGGTGGTGCCTGGGAGCTGTCTTGTATGAGATGC
TGTATGGCCTGCCGCCTTTTTATAGCCGAAACACAGCTGAAATGTACGACAACATTCTGAACAAGCCT
CTCCAGCTGAAACCAAATATTACAAATTCCGCAAGACACCTCCTGGAGGGCCTCCTGCAGAAGGACAG
GACAAAGCGGCTCGGGGCCAAGGATGACTTCATGGAGATTAAGAGTCATGTCTTCTTCTCCTTAATTA
ACTGGGATGATCTCATTAATAAGAAGATTACTCCCCCTTTTAACCCAAATGTGAGTGGGCCCAACGAC
CTACGGCACTTTGACCCCGAGTTTACCGAAGAGCCTGTCCCCAACTCCATTGGCAAGTCCCCTGACAG
CGTCCTCGTCACAGCCAGCGTCAAGGAAGCTGCCGAGGCTTTCCTAGGCTTTTCCTATGCGCCTCCCA
CGGACTCTTTCCTCCTCGAGGGC

| | | | |
|---|---|---|---|
| NOV1b, CG164221-02 | | | |
| Protein Sequence | SEQ ID NO: 4 | 431 aa | MW at 48941.8 kD |

MTVKTEAAKGTLTYSRMRGMVAILIAFMKQRRMGLNDFIQKIANNSYACKHPEVQSILKISQPQEPEL
MNANPSPPPSPSQQINLGPSSNPHAKPSDFHFLKVIGKGSFGKVLLARHKAEEVFYAVKVLQKKAILK
KKEEKHIMSERNVLLKNVKHPFLVGLHFSFQTADKLYFVLDYINGGELFYHLQRERCFLEPRARFYAA
EIASALGYLHSLNIVYRDLKPENILLDSQGHIVLTDFGLCKENIEHNSTTSTFCGTPEYLAPEVLHKQ
PYDRTVDWWCLGAVLYEMLYGLPPFYSRNTAEMYDNILNKPLQLKPNITNSARHLLEGLLQKDRTKRL
GAKDDFMEIKSHVFFSLINWDDLINKKITPPFNPNVSGPNDLRHFDPEFTEEPVPNSIGKSPDSVLVT
ASVKEAAEAFLGFSYAPPTDSFL

A ClustalW comparison of the above protein sequences yields the following sequence alignment shown in Table 1B.

TABLE 1B
Comparison of the NOV1 protein sequences.

| | |
|---|---|
| NOV1a | MTVKTEAAKGTLTYSRMRGMVAILIAFMKQRRMGLNDFIQKIANNSYACKHPEVQSILKI |
| NOV1b | MTVKTEAAKGTLTYSRMRGMVAILIAFMKQRRMGLNDFIQKIANNSYACKHPEVQSILKI |
| NOV1a | SQPQEPELMNANPSPPPSPSQQINLGPSSNPHAKPSDFHFLKVIGKGSFGKVLLARHKAE |
| NOV1b | SQPQEPELMNANPSPPPSPSQQINLGPSSNPHAKPSDFHFLKVIGKGSFGKVLLARHKAE |
| NOV1a | EVFYAVKVLQKKAILKKKEEKHIMSERNVLLKNVKHPFLVGLHFSFQTADKLYFVLDYIN |
| NOV1b | EVFYAVKVLQKKAILKKKEEKHIMSERNVLLKNVKHPFLVGLHFSFQTADKLYFVLDYIN |
| NOV1a | GGELFYHLQRERCFLEPRARFYAAEIASALGYLHSLNIVYRDLKPENILLDSQGHIVLTD |
| NOV1b | GGELFYHLQRERCFLEPRARFYAAEIASALGYLHSLNTVYRDLKPENILLDSQGHIVLTD |
| NOV1a | FGLCKENIEHNSTTSTFCGTPEYLAPEVLHKQPYDRTVDWWCLGAVLYEMLYGLPPFYSR |
| NOV1b | FGLCKENIEHNSTTSTFCGTPEYLAPEVLHKQPYDRTVDWWCLGAVLYEMLYGLPPFYSR |
| NOV1a | NTAEMYDNILNKPLQLKPNITNSARHLLEGLLQKDRTKRLGAKDDFMEIKSHVFFSLINW |
| NOV1b | NTAEMYDNILNKPLQLKPNITNSARHLLEGLLQKDRTKRLGAKDDFMEIKSHVFFSLINW |
| NOV1a | DDLINKKITPPFNPNVSGPNDLRHFDPEFTEEPVPNSIGKSPDSVLVTASVKEAAEAFLG |
| NOV1b | DDLINKKITPPFNPNVSGPNDLRHFDPEFTEEPVPNSIGKSPDSVLVTASVKEAAEAFLG |
| NOV1a | FSYAPPTDSFL |
| NOV1b | FSYAPPTDSFL |
| NOV1a | (SEQ ID NO: 2) |
| NOV1b | (SEQ ID NO: 4) |

Further analysis of the NOV1a protein yielded the following properties shown in Table 1C.

TABLE 1C
Protein Sequence Properties NOVIa

| SignalP analysis: | No Known Signal Sequence Predicted | | | |
|---|---|---|---|---|
| PSORT II analysis: | PSG: a new signal peptide prediction method | | | |
| | N-region: length 9; pos. chg 2; neg. chg 1 | | | |
| | H-region: length 6; peak value −1.48 | | | |
| | PSG score: −5.88 | | | |
| | GvH: von Heijne's method for signal seq. recognition | | | |
| | GvH score (threshold: −2.1): −12.80 | | | |
| | possible cleavage site: between 34 and 35 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = | 3.29 (at 12) | |
| | ALOM score: 3.29 (number of TMSs: 0) | | | |
| | MITDISC: discrimination of mitochondrial targeting seq | | | |
| | R content: | 4 | Hyd Moment (75): | 3.99 |
| | Hyd Moment (95): | 1.34 | G content: | 3 |
| | D/E content: | 2 | S/T content: | 5 |

```
Score: −4.17
Gavel: prediction of cleavage sites for mitochondrial preseq
R-2 motif at 42 RRM|GL
NUCDISC: discrimination of nuclear localization signals
pat4: none
pat7: none
bipartite: none
content of basic residues: 11.8%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues
Final Results (k = 9/23):
39.1%: cytoplasmic
30.4%: nuclear
21.7%: mitochondrial
  4.3%: Golgi
  4.3%: endoplasmic reticulum
>> prediction for CG164221-01 is cyt (k = 23)
```

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAW90139 | Human sgk protein - Homo | 1 . . . 431 | 431/431 (100%) | 0.0 |
| | sapiens, 431 aa. [EP887081-A2, | 1 . . . 431 | 431/431 (100%) | |
| | 30 DEC. 1998] | | | |
| AAB65613 | Novel protein kinase, SEQ ID | 1 . . . 431 | 430/431 (99%) | 0.0 |
| | NO: 139 - Homo sapiens, 431 aa. | 1 . . . 431 | 431/431 (99%) | |
| | [WO200073469-A2, 07 DEC. | | | |
| | 2000] | | | |
| AAB24115 | Human serum and glucocorticoid | 1 . . . 431 | 430/431 (99%) | 0.0 |
| | regulated kinase protein (HSGK) - | 1 . . . 431 | 431/431 (99%) | |
| | Homo sapiens, 431 aa. | | | |
| | [CN1259573-A, 12 JUL. 2000] | | | |
| AAW77217 | Human cell-volume regulating | 1 . . . 431 | 430/431 (99%) | 0.0 |
| | kinase h-sgk - Homo sapiens, | 1 . . . 431 | 431/431 (99%) | |
| | 431 aa. [EP861896-A2, 02 SEP. | | | |
| | 1998] | | | |
| AAY95279 | Human serum and | 1 . . . 431 | 429/431 (99%) | 0.0 |
| | glucocorticoid-induced protein | 1 . . . 431 | 430/431 (99%) | |
| | kinase - Homo sapiens, 431 aa. | | | |
| | [WO200035946-A1, 22 JUN. | | | |
| | 2000] | | | |

In a BLAST search of public sequence databases, the NOV1a protein was found to have homology to the proteins shown in the BLASTP data in Table 1E.

TABLE 1E
Public BLASTP Results for NOV1a

| | | NOV1a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD58123 | Sequence 1 from Patent | 1 . . . 431 | 431/431 (100%) | 0.0 |
| | WO02074987 - Homo sapiens | 1 . . . 431 | 431/431 (100%) | |
| | (Human), 431 aa. | | | |
| O00141 | Serine/threonine-protein kinase | 1 . . . 431 | 430/431 (99%) | 0.0 |
| | Sgk1 (EC 2.7.1.37) | 1 . . . 431 | 431/431 (99%) | |
| | (Serum/glucocorticoid-regulated | | | |
| | kinase 1) - Homo sapiens | | | |
| | (Human), 431 aa. | | | |
| A48094 | serum and glucocorticoid- | 1 . . . 431 | 419/431 (97%) | 0.0 |
| | regulated kinase - rat, 431 aa. | 1 . . . 431 | 426/431 (98%) | |
| Q9XT18 | Serine/threonine-protein kinase | 1 . . . 431 | 418/431 (96%) | 0.0 |
| | Sgk1 (EC 2.7.1.37) | 1 . . . 431 | 426/431 (97%) | |
| | (Serum/glucocorticoid-regulated | | | |
| | kinase 1) - Oryctolagus cuniculus | | | |
| | (Rabbit), 431 aa. | | | |
| Q9WVC6 | Serine/threonine-protein kinase | 1 . . . 431 | 416/431 (96%) | 0.0 |
| | Sgk1 (EC 2.7.1.37) | 1 . . . 431 | 425/431 (98%) | |
| | (Serum/glucocorticoid-regulated | | | |
| | kinase 1) - Mus musculus | | | |
| | (Mouse), 431 aa. | | | |

PFam analysis predicts that the NOV1a protein contains the domains shown in the Table 1F.

TABLE 1F
Domain Analysis of NOV1a

| | | Identities/ | |
|---|---|---|---|
| | | Similarities | |
| Pfam | NOV1a Match | for the | Expect |
| Domain | Region | Matched Region | Value |
| pkinase | 98 . . . 355 | 107/301 (36%) | 3.2e−88 |
| | | 212/301 (70%) | |
| pkinase_C | 356 . . . 430 | 28/77 (36%) | 2.2e−13 |
| | | 54/77 (70%) | |

### Example 2

The NOV2 clone was analyzed, and the nucleotide and encoded polypeptide sequences are shown in Table 2A.

TABLE 2A
NOV2 Sequence Analysis

NOV2a, CG180777-01 SEQ ID NO: 5 1140 bp
DNA Sequence ORF Start: ATG at 55 ORF Stop: end of sequence
ATTGGAGTTTGGAAGTTCAGGAGCACAGGAGCACAGGCCCACGACTGCAGCGGGATGGACCAGTACTG
CATCCTGGGCCGCATCGGGGAGGGCGCCCACGGCATCGTCTTCAAGGCCAAGCACGTGGAGCCGAGGG
TGGGCTGGCAGTGTCTGCCTTCTATCCTGCAGACTGGCGAGATAGTTGCCCTCAAGAAGGTGGCCCTA
AGGCGGTTGGAGGACGGCTTCCCTAACCAGGCCCTGCGGGAGATTAAGGCTCTGCAGGAGATGGAGGA
CAATCAGTATGTGGTACAACTGAAGGCTGTGTTCCCACACGGTGGAGGCTTTGTGCTGGCCTTTGAGT
TCATGCTGTCGGATCTGGCCGAGGTGGTGCGCCATGCCCAGAGGCCACTAGCCCAGGCACAGGTCAAG
AGCTACCTGCAGATGCTGCTCAAGGGTGTCGCCTTCTGCCATGCCAACAACATTGTACATCGGGACCT
GAAACCTGCCAACCTGCTCATCAGCGCCTCAGGCCAGCTCAAGATAGCGGACTTTGGCCTGGCTCGAG
TCTTTTCCCCAGACGGCAGCCACCTCTACACACACCAGGTGGCCACCAGGTGGTACCGAGCCCCCGAG
CTCCTGTATGGTGCCCGCCAGTATGACCAGGGCGTCGATCTGTGGTCTGTGGGCTGCATCATGGGGGA
GCTGTTGAATGGGTCCCCCCTTTTCCCGGGCAAGAACGATATTGAACAGCTTTGCTATGTGCTTCGCA
TCTTGGGCACCCCAAACCCTCAAGTCTGGCCGGAGCTCACTGAGCTGCCGGACTACAACAAGATCTCC
TTTAAGGAGCAGGTGCCCATGCCCCTGGAGGAGGTGCTGCCTGACGTCTCTCCCCAGGCATTGGATCT
GCTGGGTCAATTCCTTCTCTACCCTCCTCACCAGCGCATCGCAGCTTCCAAGGCTCTCCTCCATCAGT
ACTTCTTCACAGCTCCCCTGCCTGCCCATCCATCTGAGCTGCCGATTCCTCAGCGTCTAGGGGGACCT
GCCCCCAAGGCCCATCCAGGGCCCCCCCACATCCATGACTTCCACGTGGACCGGCCTCTTGAGGAGTC
GCTGTTGAACCCAGAGCTGATTCGGCCCTTCATCCTGGAGGGGTGAGAAGTT
NOV2a, CG180777-01
Protein Sequence SEQ ID NO: 6 359 aa MW at 40154.1 kD
MDQYCILGRIGEGAHGIVFKAKHVEPRVGWQCLPSILQTGEIVALKKVALRRLEDGFPNQALREIKAL
QEMEDNQYVVQLKAVFPHGGGFVLAFEFMLSDLAEVVRHAQRPLAQAQVKSYLQMLLKGVAFCHANNI
VHRDLKPANLLISASGQLKIADFGLARVFSPDGSHLYTHQVATRWYRAPELLYGARQYDQGVDLWSVG
CIMGELLNGSPLFPGKNDIEQLCYVLRILGTPNPQVWPELTELPDYNKISFKEQVPMPLEEVLPDVSP
QALDLLGQFLLYPPHQRIAASKALLHQYFFTAPLPAHPSELPIPQRLGGPAPKAHPGPPHIHDFHVDR
PLEESLLNPELIRPFILEG
NOV2b, CG180777-02 SEQ ID NO: 7 2078 bp
DNA Sequence ORF Start: ATG at 238 ORF Stop: end of sequence
CAGGTGCTGCGTGACAGCGGAGGGCTAGGAAAAGGCGCAGTGGGGCCCGGAGCTGTCACCCCTGACTC
GACGCAGCTTCCGTTCTCCTGGTGACGTGGCCTACAGGAACCGCCCCGGTGGTCAGCTGCCGCGCTGT
TGCTAGGCAACAGCGTGCGTGCTCAGATCAGCGTGGGGTGGAGGAGAAGTGGAGTTTGGAAGTTCAGG
GGCACAGGGGCACAGGCCCACGACTGCAGCGGGATGGACCAGTACTGCATCCTGGGCCGCATCGGGGA
GGGCGCCCACGGCATCGTCTTCAAGGCCAAGCACGTGGAGACTGGCGAGATAATTGCCCTCAAGAAGG
TGGCCCTAAGGCGGTTGGAAGACGGCTTCCCTAACCAGGCCCTGCGGGAGATTAAGGCTCTGCAGGAG
ATGGAGGACAATCAGTATGTGGTACAACTGAAGGCTGTGTTCCCACACGGTGGAGGCTTTGTGCTGGC
CTTTGAGTTCATGCTGTCGGATCTGGCCGAGGTGGTGCGCCATGCCCAGAGGCCACTAGCCCAGGCAC
AGGTCAAGAGCTACCTGCAGATGCTGCTCAAGGGTGTCGCCTTCTGCCATGCCAACAACATTGTACAT
CGGGACCTGAAACCTGCCAACCTGCTCATCAGCGCCTCAGGCCAGCTCAAGATAGCGGACTTTGGCCT
GGCTCGAGTCTTTTCCCCAGACGGCAGCCGCCTCTACACACACCAGGTGGCCACCAGGTCTGTGGGCT
GCATCATGGGGGAGCTGTTGAATGGGTCCCCCCTTTTCCCGGGCAAGAACGATATTGAACAGCTTTGC
TATGTGCTTCGCATCTTGGGCACCCCAAACCCTCAAGTCTGGCCGGAGCTCACTGAGCTGCCGGACTA
CAACAAGATCTCCTTTAAGGAGCAGGTGCCCATGCCCCTGGAGGAGGTGCTGCCTGACGTCTCTCCCC
AGGCATTGGATCTGCTGGGTCAATTCCTTCTCTACCCTCCTCACCAGCGCATCGCAGCTTCCAAGGCT
CTCCTCCATCAGTACTTCTTCACAGCTCCCCTGCCTGCCCATCCATCTGAGCTGCCGATTCCTCAGCG
TCTAGGGGGACCTGCCCCCAAGGCCCATCCAGGGCCCCCCCACATCCATGACTTCCACGTGGACCGGC
CTCTTGAGGGAGTCGCTGTTGAACCCAGAGCTGATTCGGCCCTTCATCCTGGAGGGGTGAGAAGTTGG
CCCTGGTCCCGTCTGCCTGCTCCTCAGGACCACTCAGTCCACCTGTTCCTCTGCCACCTGCCTGGCTT
CACCCTCCAAGGCCTCCCCATGGCCACAGTGGGCCCACACCACACCTTGCCCCTTAGCCCTTGCGAGG
GTTGGTCTCGAGGCAGAGGTCATGTTCCCAGCCAAGAGTATGAGAACATCCAGTCGAGCAGAGGAGAT
TCATGGCCTGTGCTCGGTGAGCCTTACCTTCTGTGTGCTACTGACGTACCCATCAGGACAGTGAGCTC
TGCTGCCAGTCAAGGCCTGCATATGCAGAATGACGATGCCTGCCTTGGTGCTGCTTCCCCCGAGTGCT
GCCTCCTGGTCAAGGAGAAGTGCAGAGAGTAAGGTGTCCTTATGTTGGAAACTCAAGTGGAAGGAAGA
TTTGGTTTGGTTTTATTCTCAGAGCCATTAAACACTAGTTCAGTATGTGAGATATAGATTCTAAAAAC
CTCAGGTGGTCTGCTTAGTCTGTCTTCTTCCTTTCTCAAGGGAAATGGCTAAGGTGGCATTGTCTCAT
GGCTCTCGTTTTTGGGGTCATGGGGAGGGTAGCACCAGCATAGCCACTTTTGCCCTGAGGGACTCCTG
TGTGCTTCACATCACTGAGCACTCATTTAGAAGTGAGGGAGACAGAAGTCTAGGCCCAGGGATGGCTC
CAGTTGGGGATCCAGCAGGAGACCCTCTGCACATGAGGCTGGTTTACCAACATCTACTCCCTCAGGAT
GAGCGTGAGCCAGAAGCAGCTGTGTATTTAAGGAAACAAGCGTTCCTGGAATTAATTTATAAATTTAA
TAAATCCCAATATAATCCCAAAAAAAAAAAAAAAAAAA
NOV2b, CG180777-02
Protein Sequence SEQ ID NO: 8 452 aa MW at 49710.7 kD
MDQYCILGRIGEGAHGIVFKAKHVETGEIIALKKVALRRLEDGFPNQALREIKALQEMEDNQYVVQLK
AVFPHGGGFVLAFEFMLSDLAEVVRHAQRPLAQAQVKSYLQMLLKGVAFCHANNIVHRDLKPANLLIS
ASGQLKIADFGLARVFSPDGSRLYTHQVATRSVGCIMGELLNGSPLFPGKNDIEQLCYVLRILGTPNP
QVWPELTELPDYNKISFKEQVPMPLEEVLPDVSPQALDLLGQFLLYPPHQRIAASKALLHQYFFTAPL
PAHPSELPIPQRLGGPAPKAHPGPPHIHDFHVDRPLEGVAVEPRADSALHPGGVRSWPWSRLPAPQDH
SVHLFLCHLPGFTLQGLPMATVGPHHTLPLSPCEGWSRGRGHVPSQEYENIQSSRGDSWPVLGEPYLL
CATDVPIRTVSSAASQGLHMQNDDACLGAASPECCLLVKEKCRE

A ClustalW comparison of the above protein sequences yields the following sequence alignment shown in Table 2B.

TABLE 2B
Comparison of the NOV2 protein sequences.

| | |
|---|---|
| NOV2a | MDQYCILGRIGEGAHGIVFKAKHVEPRVGWQCLPSILQTGEIVALKKVALRRLEDGFPNQ |
| NOV2b | MDQYCILGRIGEGAHGIVFKAKHVE-------------TGEIIALKKVALRRLEDGFPNQ |
| NOV2a | ALREIKALQEMEDNQYVVQLKAVFPHGGGFVLAFEFMLSDLAEVVRHAQRPLAQAQVKSY |
| NOV2b | ALREIKALQEMEDNQYVVQLKAVFPHGGGFVLAFEFMLSDLAEVVRHAQRPLAQAQVKSY |
| NOV2a | LQMLLKGVAFCHANNIVHRDLKPANLLISASGQLKIADFGLARVFSPDGSHLYTHQVATR |
| NOV2b | LQMLLKGVAFCHANNIVHRDLKPANLLISASGQLKIADFGLARVFSPDGSRLYTHQVATR |
| NOV2a | WYRAPELLYGARQYDQGVDLWSVGCIMGELLNGSPLFPGKNDIEQLCYVLRILGTPNPQV |
| NOV2b | ---------------------SVGCIMGELLNGSPLFPGKNDIEQLCYVLRILGTPNPQV |
| NOV2a | WPELTELPDYNKISFKEQVPMPLEEVLPDVSPQALDLLGQFLLYPPHQRIAASKALLHQY |
| NOV2b | WPELTELPDYNKISFKEQVPMPLEEVLPDVSPQALDLLGQFLLYPPHQRIAASKALLHQY |
| NOV2a | FFTAPLPAHPSELPIPQRLGGPAPKAHPGPPHIHDFHVDRPLE--------ESLLNPELI |
| NOV2b | FFTAPLPAHPSELPIPQRLGGPAPKAHPGPPHIHDFHVDRPLEGVAVEPRADSALHPGGV |
| NOV2a | RPFILEG----------------------------------------------------- |
| NOV2b | RSWPWSRLPAPQDHSVHLFLCHLPGFTLQGLPMATVGPHHTLPLSPCEGWSRGRGHVPSQ |
| NOV2a | ------------------------------------------------------------ |
| NOV2b | EYENIQSSRGDSWPVLGEPYLLCATDVPIRTVSSAASQGLHMQNDDACLGAASPECCLLV |
| NOV2a | ------ |
| NOV2b | KEKCRE |
| NOV2a | (SEQ ID NO: 6) |
| NOV2b | (SEQ ID NO: 8) |

Further analysis of the NOV2a protein yielded the following properties shown in Table 2C.

TABLE 2C
Protein Sequence Properties NOV2a

| SignalP analysis: | No Known Signal Sequence Predicted | | | |
|---|---|---|---|---|
| PSORT II analysis: | PSG: a new signal peptide prediction method | | | |
| | N-region: length 9; pos. chg 1; neg. chg 1 | | | |
| | H-region: length 2; peak value −0.20 | | | |
| | PSG score: −4.60 | | | |
| | GvH: von Heijne's method for signal seq. recognition | | | |
| | GvH score (threshold: −2.1): −8.44 | | | |
| | possible cleavage site: between 44 and 45 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = | 4.14 (at 146) | |
| | ALOM score: 0.37 (number of TMSs: 0) | | | |
| | MITDISC: discrimination of mitochondrial targeting seq | | | |
| | R content: | 1 | Hyd Moment (75): | 6.08 |
| | Hyd Moment (95): | 4.92 | G content: | 2 |
| | D/E content: | 2 | S/T content: | 0 |

Score: −7.23
Gavel: prediction of cleavage sites for mitochondrial preseq
cleavage site motif not found
NUCDISC: discrimination of nuclear localization signals
pat4: none
pat7: none
bipartite: none
content of basic residues: 8.9%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
47.8%: cytoplasmic
26.1%: mitochondrial
26.1%: nuclear
>> prediction for CG180777-01 is cyt (k = 23)

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY90244 | Human cyclin dependent kinase, | 1 . . . 359 | 357/359 (99%) | 0.0 |
| | hPNQALRE, protein sequence #3 - | 1 . . . 359 | 358/359 (99%) | |
| | Homo sapiens, 359 aa. | | | |
| | [WO200036118-A2, 22 JUN. | | | |
| | 2000] | | | |
| AAY90245 | Human cyclin dependent kinase, | 1 . . . 359 | 344/359 (95%) | 0.0 |
| | hPNQALRE, protein sequence #4 - | 1 . . . 345 | 344/359 (95%) | |
| | Homo sapiens, 345 aa. | | | |
| | [WO200036118-A2, 22 JUN. | | | |
| | 2000] | | | |
| AAE11784 | Human kinase (PKIN)-18 protein - | 1 . . . 359 | 345/359 (96%) | 0.0 |
| | Homo sapiens, 346 aa. | 1 . . . 346 | 345/359 (96%) | |
| | [WO200181555-A2, 01 NOV. | | | |
| | 2001] | | | |
| AAY90243 | Human cyclin dependent kinase, | 1 . . . 358 | 344/358 (96%) | 0.0 |
| | hPNQALRE, protein sequence #2 - | 1 . . . 345 | 344/358 (96%) | |
| | Homo sapiens, 346 aa. | | | |
| | [WO200036118-A2, 22 JUN. | | | |
| | 2000] | | | |
| AAM93918 | Human polypeptide, SEQ ID NO: | 1 . . . 359 | 337/359 (93%) | 0.0 |
| | 4077 - Homo sapiens, 338 aa. | 1 . . . 338 | 337/359 (93%) | |
| | [EP1130094-A2, 05 SEP. 2001] | | | |

In a BLAST search of public sequence databases, the NOV2a protein was found to have homology to the proteins shown in the BLASTP data in Table 2E.

TABLE 2E
Public BLASTP Results for NOV2a

| | | NOV2a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8IZL9 | Cell cycle related kinase - Homo | 1 . . . 359 | 345/359 (96%) | 0.0 |
| | sapiens (Human), 346 aa. | 1 . . . 346 | 345/359 (96%) | |
| Q9JHU3 | CDK-related protein kinase | 1 . . . 359 | 325/359 (90%) | 0.0 |
| | PNQLARE - Mus musculus | 1 . . . 346 | 335/359 (92%) | |
| | (Mouse), 346 aa. | | | |
| O95137 | Cell cycle related kinase (Cyclin- | 1 . . . 349 | 308/349 (88%) | e−174 |
| | dependent protein kinase H) - | 1 . . . 315 | 310/349 (88%) | |
| | Homo sapiens (Human), 452 aa. | | | |
| Q9BUF4 | Similar to cell cycle related | 1 . . . 294 | 259/294 (88%) | e−141 |
| | kinase - Homo sapiens (Human), | 1 . . . 260 | 259/294 (88%) | |
| | 275 aa. | | | |
| P29620 | CDC2+/CDC28-related protein | 2 . . . 338 | 150/338 (44%) | 2e−73 |
| | kinase R2 (EC 2.7.1.-) - Oryza | 17 . . . 330 | 200/338 (58%) | |
| | sativa (Rice), 424 aa. | | | |

PFam analysis predicts that the NOV2a protein contains the domains shown in the Table 2F.

TABLE 2F
Domain Analysis of NOV2a

| | | Identities/ | |
|---|---|---|---|
| | | Similarities | |
| Pfam | NOV2a Match | for the | Expect |
| Domain | Region | Matched Region | Value |
| pkinase | 4 . . . 301 | 104/317 (33%) | 3.7e−73 |
| | | 220/317 (69%) | |

### Example 3

The NOV3 clone was analyzed, and the nucleotide and encoded polypeptide sequences are shown in Table 3A.

TABLE 3A
NOV3 Sequence Analysis

NOV3a, CG181825-01 SEQ ID NO: 9 958 bp
DNA Sequence ORF Start: ATG at 99 ORF Stop: end of sequence
AGCCAGAGACAGGACACCAGAAGAGACAGGAGATCAGAGACCAGAGGAACAGAGAAGAGGCCCCAGAG
CAAGGCAAGGAACGGCCAAGGCACCAGGACATGGATGCAGTGGAGCCAGGAGGACGTGGCTGGGCCAG
CATGTTGGCGTGCAGGCTTTGGAAAGCCATCAGCAGGGCGCTGTTTGCAGAGTTCCTGGCCACGGGGC
TGTATGTGTTCTTTGGCGTGGGCTCAGTCATGCGCTGGCCCACAGCACTTCCCTCCGTGCTACAGATT
GCCATCACCTTCAACCTGGTCACCGCCATGGCTGTGCAGGTCACCTGGAAGGCCAGCGGGGCCCACGC
CAACCCCGCCGTGACGCTGGCCTTCCTCGTAGGCTCCCACATCTCTCTGCCCCGTGCTGTGGCCTATG
TGGCTGCCCAGCTGGTGGGGGCCACGGTGGGGGCTGCTCTGCTTTATGGGGTCATGCCGGGAGACATC
CGAGAGACCCTTGGGATCAACGTGGTCCGGAACAGTGTCTCAACTGGCCAGGCGGTGGCAGTGGAGCT
GCTTCTGACCCTGCAGCTGGTGCTCTGTGTCTTCGCTTCCACCGACAGCCGTCAGACATCAGGCTCCC
CGGCCACCATGATTGGGATCTCTGTGGCACTGGGCCACCTCATTGGGATCCACTTCACTGGCTGCTCC
ATGAATCCAGCCCGCTCCTTCGGCCCTGCCATCATCATTGGGAAGTTCACAGTCCACTGGGTCTTCTG
GGTGGGGCCCCTGATGGGAGCCCTCCTGGCCTCACTGATCTACAACTTCGTCCTGTTCCCCGACACCA
AGAACCTGGCGCAGCGGCTGGCTATCCTCACAGGCACCGTAGAGGTGGGGACAGGGGCAGGGGCAGGG
GCGGAGCCCCTGAAGAAGGAATCCCAGCCGGGTTCGGGAGCCGTGGAGATGGAGAGTGTGTGAAACAG
CCTAAC
NOV3a, CG181825-01
Protein Sequence SEQ ID NO: 10 282 aa MW at 29383.1 kD
MDAVEPGGRGWASMLACRLWKAISRALFAEFLATGLYVFFGVGSVMRWPTALPSVLQIAITFNLVTAM
AVQVTWKASGAHANPAVTLAFLVGSHISLPRAVAYVAAQLVGATVGAALLYGVMPGDIRETLGINVVR
NSVSTGQAVAVELLLTLQLVLCVFASTDSRQTSGSPATMIGISVALGHLIGIHFTGCSMNPARSFGPA
IIIGKFTVHWVFWVGPLMGALLASLIYNFVLFPDTKNLAQRLAILTGTVEVGTGAGAGAEPLKKESQP
GSGAVEMESV

Further analysis of the NOV3a protein yielded the following properties shown in Table 3B.

TABLE 3B
Protein Sequence Properties NOV3a

| SignalP analysis: | Cleavage site between residues 36 and 37 | | | | |
|---|---|---|---|---|---|
| PSORT II analysis: | PSG: a new signal peptide prediction method | | | | |
| | N-region: length 9; pos. chg 1; neg. chg 2 | | | | |
| | H-region: length 8; peak value 0.00 | | | | |
| | PSG score: −4.40 | | | | |
| | GvH: von Heijne's method for signal seq. recognition | | | | |
| | GvH score (threshold: −2.1): −5.10 | | | | |
| | possible cleavage site: between 44 and 45 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 6 | | | | |
| | INTEGRAL | Likelihood = | −1.70 | Transmembrane | 26-42 |
| | INTEGRAL | Likelihood = | −1.70 | Transmembrane | 54-70 |
| | INTEGRAL | Likelihood = | −1.06 | Transmembrane | 101-117 |
| | INTEGRAL | Likelihood = | −7.38 | Transmembrane | 144-160 |
| | INTEGRAL | Likelihood = | −0.48 | Transmembrane | 172-188 |
| | INTEGRAL | Likelihood = | −2.39 | Transmembrane | 214-230 |
| | PERIPHERAL | Likelihood = | 2.86 (at 81) | | |

| ALOM score: −7.38 (number of TMSs: 6) | | | |
|---|---|---|---|
| MTOP: Prediction of membrane topology (Hartmann et al.) | | | |
| Center position for calculation: 33 | | | |
| Charge difference: −1.0 C(1.0)-N(2.0) | | | |
| N >= C: N-terminal side will be inside | | | |
| >>> membrane topology: type 3a | | | |
| MITDISC: discrimination of mitochondrial targeting seq | | | |
| R content: | 0 | Hyd Moment (75): | 3.62 |
| Hyd Moment (95): | 8.06 | G content: | 0 |
| D/E content: | 2 | S/T content: | 0 |

Score: −7.16
Gavel: prediction of cleavage sites for mitochondrial preseq
cleavage site motif not found
NUCDISC: discrimination of nuclear localization signals
pat4: none
pat7: none
bipartite: none
content of basic residues: 5.7%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
55.6%: endoplasmic reticulum
33.3%: mitochondrial
11.1%: vesicles of secretory system
>> prediction for CG181825-01 is end (k = 9)

A search of the NOV3a protein against the Geneseq database, a proprietary database that contains sequences published in patents and patent publication, yielded several homologous proteins shown in Table 3C.

TABLE 3C
Geneseq Results for NOV3a

| | | NOV3a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB97500 | Novel human protein SEQ ID NO: | 22 . . . 254 | 138/236 (58%) | 1e−77 |
| | 768 - Homo sapiens, 265 aa. | 9 . . . 244 | 184/236 (77%) | |
| | [WO200222660-A2, 21 MAR. | | | |
| | 2002] | | | |
| ABG96280 | Human ovarian cancer marker | 22 . . . 254 | 138/236 (58%) | 1e−77 |
| | OV3 - Homo sapiens, 265 aa. | 9 . . . 244 | 184/236 (77%) | |
| | [WO200271928-A2, 19 SEP. | | | |
| | 2002] | | | |
| AAR51070 | A water channel protein localized | 22 . . . 251 | 138/232 (59%) | 2e−76 |
| | in the rat kidney collecting tubule - | 8 . . . 239 | 175/232 (74%) | |
| | Rattus sp. (Sprague-Dawley), | | | |
| | 271 aa. [EP591789-A, 13 APR. | | | |
| | 1994] | | | |
| AAW94319 | Rat aquaporin-5 - Rattus sp, 265 | 22 . . . 254 | 133/236 (56%) | 7e−75 |
| | aa. [US5858702-A, 12 JAN. 1999] | 9 . . . 244 | 181/236 (76%) | |
| AAW55787 | Rat aquaporin-5 - Rattus sp, 265 | 22 . . . 254 | 133/236 (56%) | 7e−75 |
| | aa. [US5741671-A, 21 APR. | 9 . . . 244 | 181/236 (76%) | |
| | 1998] | | | |

In a BLAST search of public sequence databases, the NOV3a protein was found to have homology to the proteins shown in the BLASTP data in Table 3D.

TABLE 3D
Public BLASTP Results for NOV3a

| | | NOV3a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q13520 | Aquaporin 6 (Aquaporin-2 like) | 1 . . . 282 | 275/282 (97%) | e−152 |
| | (hKID) - Homo sapiens (Human), | 1 . . . 282 | 275/282 (97%) | |
| | 282 aa. | | | |
| Q9WTY0 | Aquaporin-6 - Rattus norvegicus | 4 . . . 279 | 213/276 (77%) | e−117 |
| | (Rat), 276 aa. | 1 . . . 274 | 234/276 (84%) | |
| Q8C4A0 | Aquaporin 6 - Mus musculus | 4 . . . 278 | 211/275 (76%) | e−115 |
| | (Mouse), 293 aa. | 1 . . . 273 | 230/275 (82%) | |
| P41181 | Aquaporin-CD (AQP-CD) (Water | 22 . . . 251 | 148/232 (63%) | 6e−81 |
| | channel protein for renal collecting | 8 . . . 239 | 178/232 (75%) | |
| | duct) (ADH water channel) | | | |
| | (Aquaporin 2) (Collecting duct | | | |
| | water channel protein) (WCH-CD) - | | | |
| | Homo sapiens (Human), 271 aa. | | | |
| I64818 | water-channel aquaporin 2 - human, | 22 . . . 251 | 147/232 (63%) | 5e−80 |
| | 271 aa. | 8 . . . 239 | 177/232 (75%) | |

PFam analysis predicts that the NOV3a protein contains the domains shown in the Table 3E.

TABLE 3E
Domain Analysis of NOV3a

| | | Identities/ | |
|---|---|---|---|
| | | Similarities for | |
| Pfam | NOV3a Match | the Matched | Expect |
| Domain | Region | Region | Value |
| MIP | 16 . . . 231 | 109/268 (41%) | 4.3e−87 |
| | | 185/268 (69%) | |

### Example 4

The NOV4 clone was analyzed, and the nucleotide and encoded polypeptide sequences are shown in Table 4A.

TABLE 4A
NOV4 Sequence Analysis

NOV4a, CG50183-01 SEQ ID NO: 11 1067 bp
DNA Sequence ORF Start: at 3 ORF Stop: end of sequence
CTTTGGAACAGAACCAGTCAACAGATTATTATTATGAGGAAAATGAAATGAATGGCACTTATGACTAC
AGTCAATATGAACTGATCTGTATCAAAGAAGATGTCAGAGAATTTGCAAAAGTTTTCCTCCCTGTATT
CCTCACAATAGTTTTCGTCATTGGACTTGCAGGCAATTCCATGGTAGTGGCAATTTATGCCTATTACA
AGAAACAGAGAACCAAAACAGATGTGTACATCCTGAATTTGGCTGTAGCAGATTTACTCCTTCTATTC
ACTCTGCCTTTTTGGGCTGTTAATGCAGTTCATGGGTGGGTTTTAGGGAAAATAATGTGCAAAATAAC
TTCAGCCTTGTACACACTAAACTTTGTCTCTGGAATGCAGTTTCTGGCTTGTATCAGCATAGACAGAT
ATGTGGCAGTAACTAAAGTCCCCAGCCAATCAGGAGTGGGAAAACCATGCTGGATCATCTGTTTCTGT
GTCTGGATGGCTGCCATCTTGCTGAGCATACCCCAGCTGGTTTTTTATACAGTAAATGACAATGCTAG
GTGCATTCCCATTTTCCCCCGCTACCTAGGAACATCAATGAAAGCATTGATTCAAATGCTAGAGATCT
GCATTGGATTTGTAGTACCCTTTCTTATTATGGGGGTGTGCTACTTTATCACGGCAAGGACACTCATG
AAGATGCCAAACATTAAAATATCTCGACCCCTAAAAGTTCTGCTCACAGTCGTTATAGTTTTCATTGT
CACTCAACTGCCTTATAACATTGTCAAGTTCTGCCGAGCCATAGACATCATCTACTCTCTGATCACCA
GCTGCAACATGAGCAAACGCATGGACATCGCCATCCAAGTCACAGAAAGCATCGCACTCTTTCACAGC
TGCCTCAACCCAATCCTTTATGTTTTTATGGGAGCATCTTTCAAAAACTACGTTATGAAAGTGGCCAA
GAAATATGGGTCCTGGAGAAGACAGAGACAAAGTGTGGAGGAGTTTCCTTTTGATTCTGAGGGTCCTA
CAGAGCCAACCAGTACTTTTAGCATTTAAAGGTAAAACTGCTCTGCC
NOV4a, CG50183-01 SEQ ID NO: 12 348 aa MW at 39710.8 kD
Protein Sequence
LEQNQSTDYYYEENEMNGTYDYSQYELICIKEDVREFAKVFLPVFLTIVFVIGLAGNSMVVAIYAYYK
KQRTKTDVYILNLAVADLLLLFTLPFWAVNAVHGWVLGKIMCKITSALYTLNFVSGMQFLACISIDRY
VAVTKVPSQSGVGKPCWIICFCVWMAAILLSIPQLVFYTVNDNARCIPIFPRYLGTSMKALIQMLEIC
IGFVVPFLIMGVCYFITARTLMKMPNIKISRPLKVLLTVVIVFIVTQLPYNIVKFCRAIDIIYSLITS
CNMSKRMDIAIQVTESIALFHSCLNPILYVFMGASFKNYVMKVAKKYGSWRRQRQSVEEFPFDSEGPT
EPTSTFSI

Further analysis of the NOV4a protein yielded the following properties shown in Table 4B.

TABLE 4B
Protein Sequence Properties NOV4a

| SignalP analysis: | Cleavage site between residues 63 and 64 | | | | |
|---|---|---|---|---|---|
| PSORT II analysis: | PSG: a new signal peptide prediction method | | | | |
| | N-region: length 8; pos. chg 0; neg. chg 2 | | | | |
| | H-region: length 3; peak value 0.00 | | | | |
| | PSG score: −4.40 | | | | |
| | GvH: von Heijne's method for signal seq. recognition | | | | |
| | GvH score (threshold: −2.1): −2.97 | | | | |
| | possible cleavage site: between 56 and 57 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 5 | | | | |
| | INTEGRAL | Likelihood = | −9.24 | Transmembrane | 40-56 |
| | INTEGRAL | Likelihood = | −5.31 | Transmembrane | 76-92 |
| | INTEGRAL | Likelihood = | −8.55 | Transmembrane | 152-168 |
| | INTEGRAL | Likelihood = | −7.75 | Transmembrane | 203-219 |
| | INTEGRAL | Likelihood = | −5.68 | Transmembrane | 236-252 |
| | PERIPHERAL | Likelihood = | 0.69 (at 289) | | |

| ALOM score: −9.24 (number of TMSs: 5) | | | |
|---|---|---|---|
| MTOP: Prediction of membrane topology (Hartmann et al.) | | | |
| Center position for calculation: 47 | | | |
| Charge difference: 4.0 C( 3.0)-N(−1.0) | | | |
| C > N: C-terminal side will be inside | | | |
| >>> membrane topology: type 3b | | | |
| MITDISC: discrimination of mitochondrial targeting seq | | | |
| R content: | 0 | Hyd Moment (75): | 8.15 |
| Hyd Moment (95): | 2.75 | G content: | 0 |
| D/E content: | 2 | S/T content: | 2 |

Score: −6.56
Gavel: prediction of cleavage sites for mitochondrial preseq
cleavage site motif not found
NUCDISC: discrimination of nuclear localization signals
pat4: none
pat7: none
bipartite: none
content of basic residues: 8.9%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
44.4%: endoplasmic reticulum
22.2%: vacuolar
22.2%: mitochondrial
11.1%: Golgi
>> prediction for CG50183-01 is end (k = 9)

A search of the NOV4a protein against the Geneseq database, a proprietary database that contains sequences published in patents and patent publication, yielded several homologous proteins shown in Table 4C.

TABLE 4C
Geneseq Results for NOV4a

| | | NOV4a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP81716 | Human C—C chemokine receptor | 1 . . . 348 | 348/348 (100%) | 0.0 |
| | 11 protein SEQ ID NO: 607 - | 3 . . . 350 | 348/348 (100%) | |
| | Homo sapiens, 350 aa. | | | |
| | [WO200261087-A2, 08 AUG. | | | |
| | 2002] | | | |
| AAB62389 | Human chemokine receptor CCX | 1 . . . 348 | 348/348 (100%) | 0.0 |
| | CKR polypeptide - Homo | 3 . . . 350 | 348/348 (100%) | |
| | sapiens, 382 aa. [WO200127146- | | | |
| | A2, 19 APR. 2001] | | | |
| AAG67237 | Amino acid sequence of human | 1 . . . 348 | 348/348 (100%) | 0.0 |
| | chemokine receptor CCR11 - | 3 . . . 350 | 348/348 (100%) | |
| | Homo sapiens, 350 aa. | | | |
| | [WO200166598-A2, 13 SEP. | | | |
| | 2001] | | | |
| AAU08994 | Human G protein-coupled | 1 . . . 348 | 348/348 (100%) | 0.0 |
| | receptor, GPCR, 2398 - Homo | 3 . . . 350 | 348/348 (100%) | |
| | sapiens, 350 aa. [WO200164882- | | | |
| | A2, 07 SEP. 2001] | | | |
| AAG80119 | Human CCR11 protein - Homo | 1 . . . 348 | 348/348 (100%) | 0.0 |
| | sapiens, 350 aa. [WO200172830- | 3 . . . 350 | 348/348 (100%) | |
| | A2, 04 OCT. 2001] | | | |

In a BLAST search of public sequence databases, the NOV4a protein was found to have homology to the proteins shown in the BLASTP data in Table 4D.

TABLE 4D
Public BLASTP Results for NOV4a

| | | NOV4a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NPB9 | C—C chemokine receptor type 11 | 1 . . . 348 | 348/348 (100%) | 0.0 |
| | (C—C CKR-11) (CC-CKR-11) | 3 . . . 350 | 348/348 (100%) | |
| | (CCR-11) (Chemokine receptor- | | | |
| | like 1) (CCRL1) (CCX CKR) - | | | |
| | Homo sapiens (Human), 350 aa. | | | |
| CAC17062 | Sequence 1 from Patent | 1 . . . 348 | 346/348 (99%) | 0.0 |
| | WO0064941 - Homo sapiens | 3 . . . 350 | 347/348 (99%) | |
| | (Human), 350 aa. | | | |
| P35350 | C—C chemokine receptor type 11 | 1 . . . 348 | 299/348 (85%) | e−180 |
| | (C—C CKR-11) (CC—CKR-11) | 3 . . . 350 | 326/348 (92%) | |
| | (CCR-11) (Possible gustatory | | | |
| | receptor type B) (PPR1 protein) - | | | |
| | Bos taurus (Bovine), 350 aa. | | | |
| Q924I3 | Chemokine receptor CCR11 - | 1 . . . 348 | 297/348 (85%) | e−177 |
| | Mus musculus (Mouse), 350 aa. | 3 . . . 350 | 323/348 (92%) | |
| Q8C0M1 | Chemokine receptor CCR11 | 1 . . . 348 | 297/348 (85%) | e−177 |
| | homolog - Mus musculus | 3 . . . 350 | 323/348 (92%) | |
| | (Mouse), 350 aa. | | | |

PFam analysis predicts that the NOV4a protein contains the domains shown in the Table 4E.

TABLE 4E
Domain Analysis of NOV4a

| | | Identities/ | |
|---|---|---|---|
| | | Similarities for | |
| Pfam | NOV4a Match | the Matched | Expect |
| Domain | Region | Region | Value |
| 7tm_1 | 56 . . . 301 | 90/276 (33%) | 1.7e−76 |
| | | 197/276 (71%) | |

### Example 5

The NOV5 clone was analyzed, and the nucleotide and encoded polypeptide sequences are shown in Table 5A.

TABLE 5A
NOV5 Sequence Analysis

NOV5a, CG50249-01 SEQ ID NO: 13 1953 bp
DNA Sequence ORF Start: ATG at 16 ORF Stop: end of sequence
GTCTGAGTCACAGAGATGGGCAAGATCGAGAACAACGAGAGGGTGATCCTCAATGTCGGGGGCACCCG
GCACGAAACCTACCGCAGCACCCTCAAGACCCTGCCTGGAACACGCCTGGCCCTTCTTGCCTCCTCCG
AGCCCCCAGGCGACTGCTTGACCACGGCGGGCGACAAGCTGCAGCCGTCGCCGCCTCCACTGTCGCCG
CCGCCGAGAGCGCCCCCGCTGTCCCCCGGGCCAGGCGGCTGCTTCGAGGGCGGCGCGGGCAACTGCAG
TTCCCGCGGCGGCAGGGCCAGCGACCATCCCGGTGGCGGCCGCGAGTTCTTCTTCGACCGGCACCCGG
GCGTCTTCGCCTATGTGCTCAATTACTACCGCACCGGCAAGCTGCACTGCCCCGCAGACGTGTGCGGG
CCGCTCTTCGAGGAGGAGCTGGCCTTCTGGGGCATCGACGAGACCGACGTGGAGCCCTGCTGCTGGAT
GACCTACCGGCAGCACCGCGACGCCGAGGAGGCGCTGGACATCTTCGAGACCCCCGACCTCATTGGCG
GCGACCCCGGCGACGACGAGGACCTGGCGGCCAAGAGGCTGGGCATCGAGGACGCGGCGGGGCTCGGG
GGCCCGGACGGCAAATCTGGCCGCTGGAGGAGGCTGCAGCCCCGCATGTGGGCCCTCTTCGAAGACCC
CTACTCGTCCAGAGCCGCCAGGTTTATTGCTTTTGCTTCTTTATTCTTCATCCTGGTTTCAATTACAA
CTTTTTGCCTGGAAACACATGAAGCTTTCAATATTGTTAAAAACAAGACAGAACCAGTCATCAATGGC
ACAAGTGTTGTTCTACAGTATGAAATTGAAACGGATCCTGCCTTGACGTATGTAGAAGGAGTGTGTGT
GGTGTGGTTTACTTTTGAATTTTTAGTCCGTATTGTTTTTTCACCCAATAAACTTGAATTCATCAAAA
ATCTCTTGAATATCATTGACTTTGTGGCCATCCTACCTTTCTACTTAGAGGTGGGACTCAGTGGGCTG
TCATCCAAAGCTGCTAAAGATGTGCTTGGCTTCCTCAGGGTGGTAAGGTTTGTGAGGATCCTGAGAAT
TTTCAAGCTCACCCGCCATTTTGTAGGTCTGAGGGTGCTTGGACATACTCTTCGAGCTAGTACTAATG
AATTTTTGCTGCTGATAATTTTCCTGGCTCTAGGAGTTTTGATATTTGCTACCATGATCTACTATGCC
GAGAGAGTGGGAGCTCAACCTAACGACCCTTCAGCTAGTGAGCACACACAGTTCAAAAACATTCCCAT
TGGGTTCTGGTGGGCTGTAGTGACCATGACTACCCTGGGTTATGGGGATATGTACCCCCAAACATGGT
CAGGCATGCTGGTGGGAGCCCTGTGTGCTCTGGCTGGAGTGCTGACAATAGCCATGCCAGTGCCTGTC
ATTGTCAATAATTTTGGAATGTACTACTCCTTGGCAATGGCAAAGCAGAAACTTCCAAGGAAAAGAAA
GAAGCACATCCCTCCTGCTCCTCAGGCAAGCTCACCTACTTTTTGCAAGACAGAATTAAATATGGCCT
GCAATAGTACACAGAGTGACACATGTCTGGGCAAAGACAATCGACTTCTGGAACATAACAGATCAGTG
TTATCAGGTGACGACAGTACAGGAAGTGAGCCGCCACTATCACCCCCAGAAAGGCTCCCCATCAGACG
CTCTAGTACCAGAGACAAAAACAGAAGAGGGGAAACATGTTTCCTACTGACGACAGGTGATTACACGT
GTGCTTCTGATGGAGGGATCAGGAAAGGTTATGAAAAATCCCGAAGCTTAAACAACATAGCGGGCTTG
GCAGGCAATGCTCTGAGGCTCTCTCCAGTAACATCACCCTACAACTCTCCTTGTCCTCTGAGGCGCTC
TCGATCTCCCATCCCATCTATCTTGTAAACCAAACAACCAAACTGCATC
NOV5a, CG50249-01 SEQ ID NO: 14 638 aa MW at 70224.7 kD
Protein Sequence
MGKIENNERVILNVGGTRHETYRSTLKTLPGTRLALLASSEPPGDCLTTAGDKLQPSPPPLSPPPRAP
PLSPGPGGCFEGGAGNCSSRGGRASDHPGGGREFFFDRHPGVFAYVLNYYRTGKLHCPADVCGPLFEE
ELAFWGIDETDVEPCCWMTYRQHRDAEEALDIFETPDLIGGDPGDDEDLAAKRLGIEDAAGLGGPDGK
SGRWRRLQPRMWALFEDPYSSRAARFIAFASLFFILVSITTFCLETHEAFNIVKNKTEPVINGTSVVL
QYEIETDPALTYVEGVCVVWFTFEFLVRIVFSPNKLEFIKNLLNIIDFVAILPFYLEVGLSGLSSKAA
KDVLGFLRVVRFVRILRIFKLTRHFVGLRVLGHTLRASTNEFLLLIIFLALGVLIFATMIYYAERVGA
QPNDPSASEHTQFKNIPIGFWWAVVTMTTLGYGDMYPQTWSGMLVGALCALAGVLTIAMPVPVIVNNF
GMYYSLAMAKQKLPRKRKKHIPPAPQASSPTFCKTELNMACNSTQSDTCLGKDNRLLEHNRSVLSGDD
STGSEPPLSPPERLPIRRSSTRDKNRRGETCFLLTTGDYTCASDGGIRKGYEKSRSLNNIAGLAGNAL
RLSPVTSPYNSPCPLRRSRSPIPSIL
NOV5b, CG50249-02 SEQ ID NO: 15 607 bp
DNA Sequence ORF Start: ATG at 13 ORF Stop: at 604
AGATTTCCCACCATGGGCAAGATCGAGAACAACGAGAGGGTGATCCTCAATGTCGGGGGCACCCGGCA
CGAAACCTACCGCAGCACCCTCAAGACCCTGCCTGGAACACGCCTGGCCCTTCTTGCCTCCTCCGAGC
CCCCAGGCGACTGCTTGACCACAGCGGGCAACTGCAGTTCCCGCGGCGGCAGGGCCAGCGACCATCCC
GGTGGCGGCCGCGAGTTCTTCTTCGACCGGCATCCGGGCGTCTTCGCCTATGTGCTCAATTACTACCG
CACCGGCAAGCTGCACTGTCCCGCAGACGTGTGCGGGCCGCTCTTCGAGGAGGAGCTGGCCTTCTGGG
GCATCGACGAGACCGACGTGGAGCCCTGCTGCTGGATGACCTACCGGCAGCACCGCGACGCCGAGGAG
GCGCTGGACATCTTCGAGACCCCCGACCTCATTGGCGGCGACCCCGGCGACGACGAGGACCTGGCGGC
CAAGAGGCTGGGCATCGAGGACGCGGCGGGGCTCGGGGGCCCCGACGGCAAATCTGGCCGCTGGAGGA
GGCTGCAGCCCCGCATGTGGGCCCTCTTCGAAGACCCCTACTCGTCCAGAGCCGCCAGGCTCG
NOV5b, CG50249-02 SEQ ID NO: 16 197 aa MW at 21779.0 kD
Protein Sequence
MGKIENNERVILNVGGTRHETYRSTLKTLPGTRLALLASSEPPGDCLTTAGNCSSRGGRASDHPGGGR
EFFFDRHPGVFAYVLNYYRTGKLHCPADVCGPLFEEELAFWGIDETDVEPCCWMTYRQHRDAEEALDI
FETPDLIGGDPGDDEDLAAKRLGIEDAAGLGGPDGKSGRWRRLQPRMWALFEDPYSSRAAR
NOV5c, CG50249-03 SEQ ID NO: 17 1815 bp
DNA Sequence ORF Start: ATG at 13 ORF Stop: end of sequence
AGATCTCCCACCATGGGCAGATCGAGAACAACGAGAGGGTGATCCTCAATGTCGGGGGCACCCGGCA
CGAAACCTACCGCAGCACCCTCAAGACCCTGCCTGGAACACGCCTGGCCCTTCTTGCCTCCTCCGAGC
CCCCAGGCAACTGCAGTTCCCGCGGCGGCAGGGCCAGCGACCATCCCGGTGGCGGCCGCGAGTTCTTC
TTCGACCGGCACCCGGGCGTCTTCGCCTATGTGCTCAATTACTACCGCACCGGCAAGCTGCACTGCCC
CGCAGACGTGTGCGGGCCGCTCTTCGAGGAGGAGCTGGCCTTCTGGGGCATCGACGAGACCGACGTGG
AGCCCTGCTGCTGGATGACCTACCGGCAGCACCGCGACGCCGAGGAGGCGCTGGACATCTTCGAGACC
CCCGACCTCATTGGCGGCGACCCCGGCGACGACGAGGACCTGGCGGCCAAGAGGCTGGGCATCGAGGA
CGCGGCGGGGCTCGGGGGCCCCGACGGCAAATCTGGCCGCTGGAGGAGGCTGCAGCCCCGCATGTGGG
CCCTCTTCGAAGACCCCTACTCGTCCAGAGCCGCCAGGTTTATTGCTTTTGCTTCTTTATTCTTCATC
CTGGTTTCAATTACAACTTTTTGCCTGGAAACACATGAAGCTTTCAATATTGTTAAAAACAAGACAGA
ACCAGTCATCAATGGCACAAGTGTTGTTCTACAGTATGAAATTGAAACGGATCCTGCCTTGACGTATG
TAGAAGGAGTGTGTGTGGTGTGGTTTACTTTTGAATTTTTAGTCCGTATTGTTTTTTCACCCAATAAA
CTTGAATTCATCAAAAATCTCTTGAATATCATTGACTTTGTGGCCATCCTACCTTTCTACTTAGAGGT
GGGACTCAGTGGGCTGTCATCCAAAGCTGCTAAAGATGTGCTTGGCTTCCTCAGGGTGGTAAGGTTTG
TGAGGATCCTGAGAATTTTCAAGCTCACCCGCCATTTTGTAGGTCTGAGGGTGCTTGGACATACTCTT
CGAGCTAGTACTAATGAATTTTTGCTGCTGATAATTTTCCTGGCTCTAGGAGTTTTGATATTTGCTAC
CATGATCTACTATGCCGAGAGAGTGGGAGCTCAACCTAACGACCCTTCAGCTAGTGAGCACACACAGT
TCAAAAACATTCCCATTGGGTTCTGGTGGGCTGTAGTGACCATGACTACCCTGGGTTATGAGGATACG
TACCCCCAAACATGGTCAGGCATGCTGGTGGGAGCCCTGTGTGCTCTGGCTGGAGTGCTGACAATAGC
CATGCCAGTGCCTGTCATTGTCAATAATTTTGGAATGTACTACTCCTTGGCAATGGCAAAGCAGAAAC
TTCCAAGGAAAAGAAAGAAGCACATCCCTCCTGCTCCTCAGGCAAGCTCACCTACTTTTTGCAAGACA
GAATTAAATATGGCCTGCAATAGTACACAGAGTGACACATGTCTGGGCAAAGACAATCGACTTCTGGA
ACATAACAGATCAGTGTTATCAGGTGACGACAGTACAGGAAGTGAGCCGCCACTATCACCCCCAGAAA
GGCTCCCCATCAGACGCTCTAGTACCAGAGACAAAAACAGAAGAGGGGAAACATGTTTCCTACTGACG
ACAGGTGATTACACGTGTGCTTCTGATGGAGGGATCAGGAAAGGATATGAAAAATCCCGAAGCTTAAA
CAACATAGCGGGCTTGGCAGGCAATGCTCTGAGGCTCTCTCCAGTAACATCACCCTACAACTCTCCTT
GTCCTCTGAGGCGCTCTCGATCTCCCATCCCATCTATCTTGCTCGAG
NOV5c, CG50249-03 SEQ ID NO: 18 599 aa MW at 66544.6 kD
Protein Sequence
MGKIENNERVILNVGGTRHETYRSTLKTLPGTRLALLASSEPPGNCSSRGGRASDHPGGGREFFFDRH
PGVFAYVLNYYRTGKLHCPADVCGPLFEEELAFWGIDETDVEPCCWMTYRQHRDAEEALDIFETPDLI
GGDPGDDEDLAAKRLGIEDAAGLGGPDGKSGRWRRLQPRMWALFEDPYSSRAARFIAFASLFFILVSI
TTFCLETHEAFNIVKNKTEPVINGTSVVLQYEIETDPALTYVEGVCVVWFTFEFLVRIVFSPNKLEFI
KNLLNIIDFVAILPFYLEVGLSGLSSKAAKDVLGFLRVVRFVRILRIFKLTRHFVGLRVLGHTLRAST
NEFLLLIIFLALGVLIFATMIYYAERVGAQPNDPSASEHTQFKNIPIGFWWAVVTMTTLGYEDTYPQT
WSGMLVGALCALAGVLTIAMPVPVIVNNFGMYYSLAMAKQKLPRKRKKHIPPAPQASSPTFCKTELNM
ACNSTQSDTCLGKDNRLLEHNRSVLSGDDSTGSEPPLSPPERLPIRRSSTRDKNRRGETCFLLTTGDY
TCASDGGIRKGYEKSRSLNNIAGLAGNALRLSPVTSPYNSPCPLRRSRSPIPSIL
NOV5d, CG50249-04 SEQ ID NO: 19 3028 bp
DNA Sequence ORF Start: ATG at 22 ORF Stop: end of sequence
AGTCATGTCTGAGTCACAGAGATGGGCAAGATCGAGAACAACGAGAGGGTGATCCTCAATGTCGGGGG
CACCCGGCACGAAACCTACCGCAGCACCCTCAAGACCCTGCCTGGAACACGCCTGGCCCTTCTTGCCT
CCTCCGAGCCCCCAGGCGACTGCTTGACCACGGCGGGCGACAAGCTGCAGCCGTCGCCGCCTCCACTG
TCGCCGCCGCCGAGAGCGCCCCCGCTGTCCCCCGGGCCAGGCGGCTGCTTCGAGGGCGGCGCGGGCAA
CTGCAGTTCCCGCGGCGGCAGGGCCAGCGACCATCCCGGTGGCGGCCGCGAGTTCTTCTTCGACCGGC
ACCCGGGCGTCTTCGCCTATGTGCTCAATTACTACCGCACCGGCAAGCTGCACTGCCCCGCAGACGTG
TGCGGGCCGCTCTTCGAGGAGGAGCTGGCCTTCTGGGGCATCGACGAGACCGACGTGGAGCCCTGCTG
CTGGATGACCTACCGGCAGCACCGCGACGCCGAGGAGGCGCTGGACATCTTCGAGACCCCCGACCTCA
TTGGCGGCGACCCCGGCGACGACGAGGACCTGGCGGCCAAGAGGCTGGGCATCGAGGACGCGGCGGGG
CTCGGGGGCCCCGACGGCAAATCTGGCCGCTGGAGGAGGCTGCAGCCCCGCATGTGGGCCCTCTTCGA
AGACCCCTACTCGTCCAGAGCCGCCAGGTTTATTGCTTTTGCTTCTTTATTCTTCATCCTGGTTTCAA
TTACAACTTTTTGCCTGGAAACACATGAAGCTTTCAATATTGTTAAAAACAAGACAGAACCAGTCATC
AATGGCACAAGTGTTGTTCTACAGTATGAAATTGAAACGGATCCTGCCTTGACGTATGTAGAAGGAGT
GTGTGTGGTGTGGTTTACTTTTGAATTTTTAGTCCGTATTGTTTTTTCACCCAATAAACTTGAATTCA
TCAAAAATCTCTTGAATATCATTGACTTTGTGGCCATCCTACCTTTCTACTTAGAGGTGGGACTCAGT
GGGCTGTCATCCAAAGCTGCTAAAGATGTGCTTGGCTTCCTCAGGGTGGTAAGGTTTGTGAGGATCCT
GAGAATTTTCAAGCTCACCCGCCATTTTGTAGGTCTGAGGGTGCTTGGACATACTCTTCGAGCTAGTA
CTAATGAATTTTTGCTGCTGATAATTTTCCTGGCTCTAGGAGTTTTGATATTTGCTACCATGATCTAC
TATGCCGAGAGAGTGGGAGCTCAACCTAACGACCCTTCAGCTAGTGAGCACACACAGTTCAAAAACAT
TCCCATTGGGTTCTGGTGGGCTGTAGTGACCATGACTACCCTGGGTTATGGGCATATGTACCCCCAAA
CATGGTCAGGCATGCTGGTGGGAGCCCTGTGTGCTCTGGCTGGAGTGCTGACAATAGCCATGCCAGTG
CCTGTCATTGTCAATAATTTTGGAATGTACTACTCCTTGGCAATGGCAAAGCAGAAACTTCCAAGGAA
AAGAAAGAAGCACATCCCTCCTGCTCCTCAGGCAAGCTCACCTACTTTTTGCAAGACAGAATTAAATA
TGGCCTGCAATAGTACACAGAGTGACACATGTCTGGGCAAAGACAATCGACTTCTGGAACATAACAGA
TCAGTGTTATCAGGTGACGACAGTACAGGAAGTGAGCCGCCACTATCACCCCCAGAAAGGCTCCCCAT
CAGACGCTCTAGTACCAGAGACAAAAACAGAAGAGGGGAAACATGTTTCCTACTGACGACAGGTGATT
ACACGTGTGCTTCTGATGGAGGGATCAGGAAAGATAACTGCAAAGAGGTTGTCATTACTGGTTACACG
CAAGCCGAGGCCAGATCTCTTACTTAATGACTTGGGGGAAGGCACAAAACATGAGAGAAAGTGTTGTA
CAGAATTTATCATGGATTATTGACTGCTGAGAAAGGGACAGTGGAATTTAGCCATACAAAGGACTATA
CTGGAAACAGACTTCTGCTGCTGAATGTGCCCTGATGTGACCAGGTTGCACTTGGAAGAGATCCTCCG
CGTCTTCATGAGGCACTTAAAGCTTATAAAAGAACTGCGGCTGGAACTCATCTGGTGCTCCCCATGAG
AGTGCTCTGCTTGTAGACTGGCCAGTGTCCATGAAACAACTGTAAATACCAACATGTGTGCATGGGTC
AACAGTCTTGGCCATTTCTCATCAAAAGAAGCCAAATTCATGATCAACATCTCTGAAGTTTCAAGTAA
GGCCCACACTTCTTTGAATTACTCTTCATGGGCCCACATTAGGTTGTGCTGTGAATTACTTAAGGCAG
TGATACTGATGTAGTATAGTTTTGTCTTAATTTCCCTTATTTCTACTTCTTTGGTTGAATCTATGAAC
TTGATTGTATAATTTTCTTATAAATTACTGATGTAATCAGCTTGTCAATTATGTTGTGAAATTGTTAG
TATTCATTTATCAAAAATGACCTATGTTTAGTCACATATTTGTTTAGTTCTGGGAAATTGTTATAGCT
TAAATGGAACTCACCAACATTATTCATAGTTTAAGTCTTTTATCATTATTACCTCAATTATAAATATT
ACAAAAACATAATTCTGGCAATGAGAGTATTTTTTTATTCAATGATCAAGGAGCAATGTCAGTATATA
GTAGAATATCAATTAAATTATATCCTAAAATGTATATTTTGCATAAAAGAGATATTCTTTAATCAATT
ACTTTTTTGTGAGTTTTGTGGCGAATGAAGCTTGTACGTGTCTTTAAAACTGTTGTAGATGAAACTGT
ATAAGATTTTTACATCTTGCTTAATCAATATTTTCAGAGTCTATTAGTTCCCCTGGGATTCTGAATAT
AACATATAGCCTATTATAAATCCCTGTATCGTGGACCTTTTGTGAACATTTCAAGGCGCATGCACAAC
CTTGATGATAACCAGTGGAAATGTAACTAACTGAAATGAAGAATAAAAGGCAAATGAGCTGGGGATAA
ACTTGAATGTTATCTGATTAAATTACTCAAATTATT
NOV5d, CG50249-04 SEQ ID NO: 20 613 aa MW at 67598.7 kD
Protein Sequence
MGKIENNERVILNVGGTRHETYRSTLKTLPGTRLALLASSEPPGDCLTTAGDKLQPSPPPLSPPPRAP
PLSPGPGGCFEGGAGNCSSRGGRASDHPGGGREFFFDRHPGVFAYVLNYYRTGKLHCPADVCGPLFEE
ELAFWGIDETDVEPCCWMTYRQHRDAEEALDIFETPDLIGGDPGDDEDLAAKRLGIEDAAGLGGPDGK
SGRWRRLQPRMWALFEDPYSSRAARFIAFASLFFILVSITTFCLETHEAFNIVKNKTEPVINGTSVVL
QYEIETDPALTYVEGVCVVWFTFEFLVRIVFSPNKLEFIKNLLNIIDFVAILPFYLEVGLSGLSSKAA
KDVLGFLRVVRFVRILRIFKLTRHFVGLRVLGHTLRASTNEFLLLIIFLALGVLIFATMIYYAERVGA
QPNDPSASEHTQFKNIPIGFWWAVVTMTTLGYGDMYPQTWSGMLVGALCALAGVLTIAMPVPVIVNNF
GMYYSLAMAKQKLPRKRKKHIPPAPQASSPTFCKTELNMACNSTQSDTCLGKDNRLLEHNRSVLSGDD
STGSEPPLSPPERLPIRRSSTRDKNRRGETCFLLTTGDYTCASDGGIRKDNCKEVVITGYTQAEARSL
T

A ClustalW comparison of the above protein sequences yields the following sequence alignment shown in Table 5B.

TABLE 5B
Comparison of the NOV5 protein sequences.

| | |
|---|---|
| NOV5a | MGKIENNERVILNVGGTRHETYRSTLKTLPGTRLALLASSEPPGDCLTTAGDKLQPSPPP |
| NOV5b | MGKIENNERVILNVGGTRHETYRSTLKTLPGTRLALLASSEPPGDCLTTA---------- |
| NOV5c | MGKIENNERVILNVGGTRHETYRSTLKTLPGTRLALLASSEPPG---------------- |
| NOV5d | MGKIENNERVILNVGGTRHETYRSTLKTLPGTRLALLASSEPPGDCLTTAGDKLQPSPPP |
| NOV5a | LSPPPRAPPLSPGPGGCFEGGAGNCSSRGGRASDHPGGGREFFFDRHPGVFAYVLNYYRT |
| NOV5b | -----------------GN-----CSSRGGRASDHPGGGREFFFDRHPGVFAYVLNYYRT |
| NOV5c | -----------------------NCSSRGGRASDHPGGGREFFFDRHPGVFAYVLNYYRT |
| NOV5d | LSPPPRAPPLSPGPGGCFEGGAGNCSSRGGRASDHPGGGREFFFDRHPGVFAYVLNYYRT |
| NOV5a | GKLHCPADVCGPLFEEELAFWGIDETDVEPCCWMTYRQHRDAEEALDIFETPDLIGGDPG |
| NOV5b | GKLHCPADVCGPLFEEELAFWGIDETDVEPCCWMTYRQHRDAEEALDIFETPDLIGGDPG |
| NOV5c | GKLHCPADVCGPLFEEELAFWGIDETDVEPCCWMTYRQHRDAEEALDIFETPDLIGGDPG |
| NOV5d | GKLHCPADVCGPLFEEELAFWGIDETDVEPCCWMTYRQHRDAEEALDIFETPDLIGGDPG |
| NOV5a | DDEDLAAKRLGIEDAAGLGGPDGKSGRWRRLQPRMWALFEDPYSSRAARFIAFASLFFIL |
| NOV5b | DDEDLAAKRLGIEDAAGLGGPDGKSGRWRRLQPRMWALFEDPYSSRAAR----------- |
| NOV5c | DDEDLAAKRLGIEDAAGLGGPDGKSGRWRRLQPRMWALFEDPYSSRAARFIAFASLFFIL |
| NOV5d | DDEDLAAKRLGIEDAAGLGGPDGKSGRWRRLQPRMWALFEDPYSSRAARFIAFASLFFIL |
| NOV5a | VSITTFCLETHEAFNIVKNKTEPVINGTSVVLQYEIETDPALTYVEGVCVVWFTFEFLVR |
| NOV5b | ------------------------------------------------------------ |
| NOV5c | VSITTFCLETHEAFNIVKNKTEPVINGTSVVLQYEIETDPALTYVEGVCVVWFTFEFLVR |
| NOV5d | VSITTFCLETHEAFNIVKNKTEPVINGTSVVLQYEIETDPALTYVEGVCVVWFTFEFLVR |
| NOV5a | IVFSPNKLEFIKNLLNIIDFVAILPFYLEVGLSGLSSKAAKDVLGFLRVVRFVRILRIFK |
| NOV5b | ------------------------------------------------------------ |
| NOV5c | IVFSPNKLEFIKNLLNIIDFVAILPFYLEVGLSGLSSKAAKDVLGFLRVVRFVRILRIFK |
| NOV5d | IVFSPNKLEFIKNLLNIIDFVAILPFYLEVGLSGLSSKAAKDVLGFLRVVRFVRILRIFK |
| NOV5a | LTRHFVGLRVLGHTLRASTNEFLLLIIFLALGVLIFATMIYYAERVGAQPNDPSASEHTQ |
| NOV5b | ------------------------------------------------------------ |
| NOV5c | LTRHFVGLRVLGHTLRASTNEFLLLIIFLALGVLIFATMIYYAERVGAQPNDPSASEHTQ |
| NOV5d | LTRHFVGLRVLGHTLRASTNEFLLLIIFLALGVLIFATMIYYAERVGAQPNDPSASEHTQ |
| NOV5a | FKNIPIGFWWAVVTMTTLGYGDMYPQTWSGMLVGALCALAGVLTIAMPVPVIVNNFGMYY |
| NOV5b | ------------------------------------------------------------ |
| NOV5c | FKNIPIGFWWAVVTMTTLGYEDTYPQTWSGMLVGALCALAGVLTIAMPVPVIVNNFGMYY |
| NOV5d | FKNIPIGFWWAVVTMTTLGYGDMYPQTWSGMLVGALCALAGVLTIAMPVPVIVNNFGMYY |
| NOV5a | SLAMAKQKLPRKRKKHIPPAPQASSPTFCKTELNMACNSTQSDTCLGKDNRLLEHNRSVL |
| NOV5b | ------------------------------------------------------------ |
| NOV5c | SLAMAKQKLPRKRKKHIPPAPQASSPTFCKTELNMACNSTQSDTCLGKDNRLLEHNRSVL |
| NOV5d | SLAMAKQKLPRKRKKHIPPAPQASSPTFCKTELNMACNSTQSDTCLGKDNRLLEHNRSVL |
| NOV5a | SGDDSTGSEPPLSPPERLPIRRSSTRDKNRRGETCFLLTTGDYTCASDGGIRKGYEKSRS |
| NOV5b | ------------------------------------------------------------ |
| NOV5c | SGDDSTGSEPPLSPPERLPIRRSSTRDKNRRGETCFLLTTGDYTCASDGGIRKGYEKSRS |
| NOV5d | SGDDSTGSEPPLSPPERLPIRRSSTRDKNRRGETCFLLTTGDYTCASDGGIRKDNCKEVV |
| NOV5a | LNNIAGLAGNALRLSPVTSPYNSPCPLRRSRSPIPSIL |
| NOV5b | -------------------------------------- |
| NOV5c | LNNIAGLAGNALRLSPVTSPYNSPCPLRRSRSPIPSIL |
| NOV5d | ITGYTQAEARSLT------------------------- |
| NOV5a | (SEQ ID NO: 14) |
| NOV5b | (SEQ ID NO: 16) |
| NOV5c | (SEQ ID NO: 18) |
| NOV5d | (SEQ ID NO: 20) |

Further analysis of the NOV5a protein yielded the following properties shown in Table 5C.

TABLE 5C
Protein Sequence Properties NOVSa

| SignalP analysis: | No Known Signal Sequence Predicted | | | | |
|---|---|---|---|---|---|
| PSORT II analysis: | PSG: a new signal peptide prediction method | | | | |
| | N-region: length 9; pos. chg 2; neg. chg 2 | | | | |
| | H-region: length 8; peak value 4.97 | | | | |
| | PSG score: 0.57 | | | | |
| | GvH: von Heijne's method for signal seq. recognition | | | | |
| | GvH score (threshold: −2.1): −3.54 | | | | |
| | possible cleavage site: between 46 and 47 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 6 | | | | |
| | INTEGRAL | Likelihood = | −6.90 | Transmembrane | 230-246 |
| | INTEGRAL | Likelihood = | −3.24 | Transmembrane | 287-303 |
| | INTEGRAL | Likelihood = | −2.23 | Transmembrane | 314-330 |
| | INTEGRAL | Likelihood = | −0.16 | Transmembrane | 343-359 |
| | INTEGRAL | Likelihood = | −13.00 | Transmembrane | 382-398 |
| | INTEGRAL | Likelihood = | −7.01 | Transmembrane | 451-467 |
| | PERIPHERAL | Likelihood = | 3.61 (at 424) | | |

| ALOM score: −13.00 (number of TMSs: 6) | | | |
|---|---|---|---|
| MTOP: Prediction of membrane topology (Hartmann et al.) | | | |
| Center position for calculation: 237 | | | |
| Charge difference: −0.5 C(−0.5)-N( 0.0) | | | |
| N >= C: N-terminal side will be inside | | | |
| >>> membrane topology: type 3a | | | |
| MITDISC: discrimination of mitochondrial targeting seq | | | |
| R content: | 0 | Hyd Moment (75): | 5.56 |
| Hyd Moment (95): | 3.52 | G content: | 1 |
| D/E content: | 2 | S/T content: | 0 |

Score: −7.90
Gavel: prediction of cleavage sites for mitochondrial preseq
cleavage site motif not found
NUCDISC: discrimination of nuclear localization signals
pat4: PRKR (4) at 490
pat4: RKRK (5) at 491
pat4: KRKK (5) at 492
pat4: RKKH (3) at 493
pat7: PRKRKKH (5) at 490
pat7: PLRRSRS (4) at 626
bipartite: none
content of basic residues: 10.7%
NLS Score: 1.37
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 89
COIL: Lupas's algorithm to detect coiled-coil regions
total: 0 residues
Final Results (k = 9/23):
66.7%: endoplasmic reticulum
22.2%: mitochondrial
11.1%: nuclear
>> prediction for CG50249-01 is end (k = 9)

A search of the NOV5a protein against the Geneseq database, a proprietary database that contains sequences published in patents and patent publication, yielded several homologous proteins shown in Table 5D.

TABLE 5D
Geneseq Results for NOV5a

| | | NOV5a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB78396 | Longer splice variant of a human | 1 . . . 638 | 638/638 (100%) | 0.0 |
| | voltage-gated potassium channel - | 1 . . . 638 | 638/638 (100%) | |
| | Homo sapiens, 638 aa. | | | |
| | [GB2372503-A, 28 AUG. 2002] | | | |
| AAO14201 | Human transporter and ion | 1 . . . 638 | 638/638 (100%) | 0.0 |
| | channel TRICH-18 - Homo | 1 . . . 638 | 638/638 (100%) | |
| | sapiens, 638 aa. [WO200204520- | | | |
| | A2, 17 JAN. 2002] | | | |
| ABP52157 | Human 53763 transporter protein | 1 . . . 638 | 638/638 (100%) | 0.0 |
| | SEQ ID NO: 11 - Homo sapiens, | 1 . . . 638 | 638/638 (100%) | |
| | 638 aa. [WO200255701-A2, 18 | | | |
| | JUL. 2002] | | | |
| ABG70285 | Human novel polypeptide #1 - | 1 . . . 638 | 638/638 (100%) | 0.0 |
| | Homo sapiens, 638 aa. | 1 . . . 638 | 638/638 (100%) | |
| | [WO200257452-A2, 25 JUL. | | | |
| | 2002] | | | |
| ABB78396 | Longer splice variant of a human | 1 . . . 638 | 638/638 (100%) | 0.0 |
| | voltage-gated potassium channel - | 1 . . . 638 | 638/638 (100%) | |
| | Homo sapiens, 638 aa. | | | |
| | [GB2372503-A, 28 AUG. 2002] | | | |

In a BLAST search of public sequence databases, the NOV5a protein was found to have homology to the proteins shown in the BLASTP data in Table 5E.

TABLE 5E
Public BLASTP Results for NOV5a

| | | NOV5a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96PR1 | Voltage gated potassium channel | 1 . . . 638 | 638/638 (100%) | 0.0 |
| | Kv3.2b (Potassium voltage-gated | 1 . . . 638 | 638/638 (100%) | |
| | potassium channel subfamily C | | | |
| | member 2) - Homo sapiens | | | |
| | (Human), 638 aa. | | | |
| P22462 | Potassium voltage-gated channel | 1 . . . 638 | 623/638 (97%) | 0.0 |
| | subfamily C member 2 (Potassium | 1 . . . 638 | 625/638 (97%) | |
| | channel Kv3.2) (KSHIIIA) - | | | |
| | Rattus norvegicus (Rat), 638 aa. | | | |
| Q96PR0 | Voltage gated potassium channel | 1 . . . 593 | 593/593 (100%) | 0.0 |
| | Kv3.2a - Homo sapiens (Human), | 1 . . . 593 | 593/593 (100%) | |
| | 613 aa. | | | |
| A39402 | potassium channel protein IIIA | 1 . . . 593 | 578/593 (97%) | 0.0 |
| | form 1, shaker-type - rat, 613 aa. | 1 . . . 593 | 580/593 (97%) | |
| S22703 | voltage-gated potassium channel | 1 . . . 593 | 577/593 (97%) | 0.0 |
| | protein Raw1 - rat, 624 aa. | 1 . . . 593 | 579/593 (97%) | |

PFam analysis predicts that the NOV5a protein contains the domains shown in the Table 5F.

TABLE 5F
Domain Analysis of NOV5a

| | | Identities/ | |
|---|---|---|---|
| | | Similarities | |
| Pfam | NOV5a Match | for the | Expect |
| Domain | Region | Matched Region | Value |
| K_tetra | 9 . . . 156 | 50/161 (31%) | 3.2e−42 |
| | | 121/161 (75%) | |
| ion_trans | 281 . . . 472 | 53/233 (23%) | 6.3e−43 |
| | | 155/233 (67%) | |

### Example 6

The NOV6 clone was analyzed, and the nucleotide and encoded polypeptide sequences are shown in Table 6A.

TABLE 6A
NOV6 Sequence Analysis

NOV6a, CG54236-02 SEQ ID NO: 21 1193 bp
DNA Sequence ORF Start: ATG at 105 ORF Stop: end of sequence
TGCTCCCTGTTTCATTAAAACCTAGAGAGATGTAATCAGTAAGCAAGAAGGAAAAAGGGAAATTCACA
AAGTAACTTTTTGTGTCTGTTTCTTTTTAACCCAGCATGGAGAGAAAATTTATGTCCTTGCAACCATC
CATCTCCGTATCAGAAATGGAACCAAATGGCACCTTCAGCAATAACAACAGCAGGAACTGCACAATTG
AAAACTTCAAGAGAGAATTTTTCCCAATTGTATATCTGATAATATTTTTCTGGGGAGTCTTGGGAAAT
GGGTTGTCCATATATGTTTTCCTGCAGCCTTATAAGAAGTCCACATCTGTGAACGTTTTCATGCTAAA
TCTGGCCATTTCAGATCTCCTGTTCATAAGCACGCTTCCCTTCAGGGCTGACTATTATCTTAGAGGCT
CCAATTGGATATTTGGAGACCTGGCCTGCAGGATTATGTCTTATTCCTTGTATGTCAACATGTACAGC
AGTATTTATTTCCTGACCGTGCTGAGTGTTGTGCGTTTCCTGGCAATGGTTCACCCCTTTCGGCTTCT
GCATGTCACCAGCATCAGGAGTGCCTGGATCCTCTGTGGGATCATATGGATCCTTATCATGGCTTCCT
CAATAATGCTCCTGGACAGTGGCTCTGAGCAGAACGGCAGTGTCACATCATGCTTAGAGCTGAATCTC
TATAAAATTGCTAAGCTGCAGACCATGAACTATATTGCCTTGGTGGTGGGCTGCCTGCTGCCATTTTT
CACACTCAGCATCTGTTATCTGCTGATCATTCGGGTTCTGTTAAAAGTGGAGGTCCCAGAATCGGGGC
TGCGGGTTTCTCACAGGAAGGCACTGACCACCATCATCATCACCTTGATCATCTTCTTCTTGTGTTTC
CTGCCCTATCACACACTGAGGACCGTCCACTTGACGACATGGAAAGTGGGTTTATGCAAAGACAGACT
GCATAAAGCTTTGGTTATCACACTGGCCTTGGCAGCAGCCAATGCCTGCTTCAATCCTCTGCTCTATT
ACTTTGCTGGGGAGAATTTTAAGGACAGACTAAAGTCTGCACTCAGAAAAGGCCATCCACAGAAGGCA
AAGACAAAGTGTGTTTTCCCTGTTAGTGTGTGGTTGAGAAAGGAAACAAGAGTATAAGGAGCTCTTAG
ATGAGACCTGTTCTTGTATCCTTGTGTCCATCTTCAT
NOV6a, CG54236-02 SEQ ID NO: 22 346 aa MW at 39634.7 kD
Protein Sequence
MERKFMSLQPSISVSEMEPNGTFSNNNSRNCTIENFKREFFPIVYLIIFFWGVLGNGLSIYVFLQPYK
KSTSVNVFMLNLAISDLLFISTLPFRADYYLRGSNWIFGDLACRIMSYSLYVNMYSSIYFLTVLSVVR
FLAMVHPFRLLHVTSIRSAWILCGIIWILIMASSIMLLDSGSEQNGSVTSCLELNLYKIAKLQTMNYI
ALVVGCLLPFFTLSICYLLIIRVLLKVEVPESGLRVSHRKALTTIIITLIIFFLCFLPYHTLRTVHLT
TWKVGLCKDRLHKALVITLALAAANACFNPLLYYFAGENFKDRLKSALRKGHPQKAKTKCVFPVSVWL
RKETRV
NOV6b, CG54236-01 SEQ ID NO: 23 1260 bp
DNA Sequence ORF Start: ATG at 105 ORF Stop: end of sequence
TGCTCCCTGTTTCATTAAAACCTAGAGAGATGTAATCAGTAAGCAAGAAGGAAAAAGGGAAATTCACA
AAGTAACTTTTTGTGTCTGTTTCTTTTTAACCCAGCATGGAGAGAAAATTTATGTCCTTGCAACCATC
CATCTCCGTATCAGAAATGGAACCAAATGGCACCTTCAGCAATAACAACAGCAGGAACTGCACAATTG
AAAACTTCAAGAGAGAATTTTTCCCAATTGTATATCTGATAATATTTTTCTGGGGAGTCTTGGGAAAT
GGGTTGTCCATATATGTTTTCCTGCAGCCTTATAAGAAGTCCACATCTGTGAACGTTTTCATGCTAAA
TCTGGCCATTTCAGATCTCCTGTTCATAAGCACGCTTCCCTTCAGGGCTGACTATTATCTTAGAGGCT
CCAATTGGATATTTGGAGACCTGGCCTGCAGGATTATGTCTTATTCCTTGTATGTCAACATGTACAGC
AGTATTTATTTCCTGACCGTGCTGAGTGTTGTGCGTTTCCTGGCAATGGTTCACCCCTTTCGGCTTCT
GCATGTCACCAGCATCAGGAGTGCCTGGATCCTCTGTGGGATCATATGGATCCTTATCATGGCTTCCT
CAATAATGCTCCTGGACAGTGGCTCTGAGCAGAACGGCAGTGTCACATCATGCTTAGAGCTGAATCTC
TATAAAATTGCTAAGCTGCAGACCATGAACTATATTGCCTTGGTGGTGGGCTGCCTGCTGCCATTTTT
CACACTCAGCATCTGTTATCTGCTGATCATTCGGGTTCTGTTAAAAGTGGAGGTCCCAGAATCGGGGC
TGCGGGTTTCTCACAGGAAGGCACTGACCACCATCATCATCACCTTGATCATCTTCTTCTTGTGTTTC
CTGCCCTATCACACACTGAGGACCGTCCACTTGACGACATGGAAAGTGGGTTTATGCAAAGACAGACT
GCATAAAGCTTTGGTTATCACACTGGCCTTGGCAGCAGCCAATGCCTGCTTCAATCCTCTGCTCTATT
ACTTTGCTGGGGAGAATTTTAAGGACAGACTAAAGTCTGCACTCAGAAAAGGCCATCCACAGAAGGCA
AAGACAAAGTGTGTTTTCCCTGTTAGTGTGTGGTTGAGAAAGGAAACAAGAGTATAAGGAGCTCTTAG
ATGAGACCTGTTCTTGTATCCTTGTGTCCATCTTCATTCACTCATAGTCTCCAAATGACTTTGTATTT
ACATCACTCCCAACAAATGTTGATTCTTAATATTTA
NOV6b, CG54236-01 SEQ ID NO: 24 346 aa MW at 39634.7 kD
Protein Sequence
MERKFMSLQPSISVSEMEPNGTFSNNNSRNCTIENFKREFFPIVYLIIFFWGVLGNGLSIYVFLQPYK
KSTSVNVFMLNLAISDLLFISTLPFRADYYLRGSNWIFGDLACRIMSYSLYVNMYSSIYFLTVLSVVR
FLAMVHPFRLLHVTSIRSAWILCGIIWILIMASSIMLLDSGSEQNGSVTSCLELNLYKIAKLQTMNYI
ALVVGCLLPFFTLSICYLLIIRVLLKVEVPESGLRVSHRKALTTIIITLIIFFLCFLPYHTLRTVHLT
TWKVGLCKDRLHKALVITLALAAANACFNPLLYYFAGENFKDRLKSALRKGHPQKAKTKCVFPVSVWL
RKETRV

A ClustalW comparison of the above protein sequences yields the following sequence alignment shown in Table 6B.

TABLE 6B
Comparison of the NOV6 protein sequences.

| | |
|---|---|
| NOV6a | MERKFMSLQPSISVSEMEPNGTFSNNNSRNCTIENFKREFFPIVYLIIFFWGVLGNGLSI |
| NOV6b | MERKFMSLQPSISVSEMEPNGTFSNNNSRNCTIENFKREFFPIVYLIIFFWGVLGNGLSI |
| NOV6a | YVFLQPYKKSTSVNVFMLNLAISDLLFISTLPFRADYYLRGSNWIFGDLACRIMSYSLYV |
| NOV6b | YVFLQPYKKSTSVNVFMLNLAISDLLFISTLPFRADYYLRGSNWIFGDLACRIMSYSLYV |
| NOV6a | NMYSSIYFLTVLSVVRFLAMVHPFRLLHVTSIRSAWILCGIIWILIMASSIMLLDSGSEQ |
| NOV6b | NMYSSIYFLTVLSVVRFLAMVHPFRLLHVTSIRSAWILCGIIWILIMASSIMLLDSGSEQ |
| NOV6a | NGSVTSCLELNLYKIAKLQTMNYIALVVGCLLPFFTLSICYLLIIRVLLKVEVPESGLRV |
| NOV6b | NGSVTSCLELNLYKIAKLQTMNYIALVVGCLLPFFTLSICYLLIIRVLLKVEVPESGLRV |
| NOV6a | SHRKALTTIIITLIIFFLCFLPYHTLRTVHLTTWKVGLCKDRLHKALVITLALAAANACF |
| NOV6b | SHRKALTTIIITLIIFFLCFLPYHTLRTVHLTTWKVGLCKDRLHKALVITLALAAANACF |
| NOV6a | NPLLYYFAGENFKDRLKSALRKGHPQKAKTKCVFPVSVWLRKETRV |
| NOV6b | NPLLYYFAGENFKDRLKSALRKGHPQKAKTKCVFPVSVWLRKETRV |
| NOV6a | (SEQ ID NO: 22) |
| NOV6b | (SEQ ID NO: 24) |

Further analysis of the NOV6a protein yielded the following properties shown in Table 6C.

TABLE 6C
Protein Sequence Properties NOV6a

| SignalP analysis: | Cleavage site between residues 60 and 61 | | | | |
|---|---|---|---|---|---|
| PSORT II analysis: | PSG: a new signal peptide prediction method | | | | |
| | N-region: length 4; pos. chg 2; neg. chg 1 | | | | |
| | H-region: length 11; peak value 5.18 | | | | |
| | PSG score: 0.78 | | | | |
| | GvH: von Heijne's method for signal seq. recognition | | | | |
| | GvH score (threshold: −2.1): −0.37 | | | | |
| | possible cleavage site: between 55 and 56 | | | | |
| | >>> Seems to have a cleavable signal peptide (1 to 55) | | | | |
| | ALOM: Klein et al's method for TM region allocation | | | | |
| | Init position for calculation: 56 | | | | |
| | Tentative number of TMS(s) for the threshold 0.5: 6 | | | | |
| | INTEGRAL | Likelihood = | −2.81 | Transmembrane | 75-91 |
| | INTEGRAL | Likelihood = | −3.72 | Transmembrane | 125-141 |
| | INTEGRAL | Likelihood = | −8.86 | Transmembrane | 157-173 |
| | INTEGRAL | Likelihood = | −7.75 | Transmembrane | 204-220 |
| | INTEGRAL | Likelihood = | −11.36 | Transmembrane | 245-261 |
| | INTEGRAL | Likelihood = | −1.06 | Transmembrane | 287-303 |
| | PERIPHERAL | Likelihood = | 2.38 (at 222) | | |

| ALOM score: −11.36 (number of TMSs: 6) | | | |
|---|---|---|---|
| MTOP: Prediction of membrane topology (Hartmann et al.) | | | |
| Center position for calculation: 27 | | | |
| Charge difference: 2.0 C(1.0)-N(−1.0) | | | |
| C > N: C-terminal side will be inside | | | |
| >>>Caution: Inconsistent mtop result with signal peptide | | | |
| >>> membrane topology: type 3b | | | |
| MITDISC: discrimination of mitochondrial targeting seq | | | |
| R content: | 1 | Hyd Moment (75): | 11.72 |
| Hyd Moment (95): | 9.21 | G content: | 0 |
| D/E content: | 2 | S/T content: | 4 |

Score: −3.79
Gavel: prediction of cleavage sites for mitochondrial preseq
R-2 motif at 13 ERK|FM
NUCDISC: discrimination of nuclear localization signals
pat4: none
pat7: none
bipartite: none
content of basic residues: 10.4%
NLS Score: −0.47
KDEL: ER retention motif in the C-terminus: none
ER Membrane Retention Signals:
XXRR-like motif in the N-terminus: ERKF
KKXX-like motif in the C-terminus: KETR
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
66.7%: endoplasmic reticulum
11.1%: Golgi
11.1%: vacuolar
11.1%: cytoplasmic
>> prediction for CG54236-02 is end (k = 9)

A search of the NOV6a protein against the Geneseq database, a proprietary database that contains sequences published in patents and patent publication, yielded several homologous proteins shown in Table 6D.

TABLE 6D
Geneseq Results for NOV6a

| | | NOV6a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP81707 | Human cysteinyl leukotriene | 1 . . . 346 | 346/346 (100%) | 0.0 |
| | CYSLT2 receptor protein SEQ ID | 1 . . . 346 | 346/346 (100%) | |
| | NO: 589 - Homo sapiens, 346 aa. | | | |
| | [WO200261087-A2, 08 AUG. | | | |
| | 2002] | | | |
| ABP95624 | Human GPCR polypeptide SEQ | 1 . . . 346 | 346/346 (100%) | 0.0 |
| | ID NO 58 - Homo sapiens, 346 | 1 . . . 346 | 346/346 (100%) | |
| | aa. [WO200216548-A2, 28 FEB. | | | |
| | 2002] | | | |
| ABU11923 | Human G-protein coupled | 1 . . . 346 | 346/346 (100%) | 0.0 |
| | receptor HGPRBMY11v1 - | 1 . . . 346 | 346/346 (100%) | |
| | Homo sapiens, 346 aa. | | | |
| | [WO200286123-A2, 31 OCT. | | | |
| | 2002] | | | |
| ABB05229 | Human LTD4-like G protein- | 1 . . . 346 | 346/346 (100%) | 0.0 |
| | coupled receptor protein SEQ ID | 1 . . . 346 | 346/346 (100%) | |
| | NO: 2 - Homo sapiens, 346 aa. | | | |
| | [WO200194580-A1, 13 DEC. | | | |
| | 2001] | | | |
| AAG77965 | Human G-protein coupled | 1 . . . 346 | 346/346 (100%) | 0.0 |
| | receptor PFI-017* - Homo | 1 . . . 346 | 346/346 (100%) | |
| | sapiens, 346 aa. [US2001039037- | | | |
| | A1, 08 NOV. 2001] | | | |

In a BLAST search of public sequence databases, the NOV6a protein was found to have homology to the proteins shown in the BLASTP data in Table 6E.

TABLE 6E
Public BLASTP Results for NOV6a

| | | NOV6a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NS75 | Cysteinyl leukotriene receptor 2 | 1 . . . 346 | 346/346 (100%) | 0.0 |
| | (CysLTR2) (PSEC0146) (HG57) | 1 . . . 346 | 346/346 (100%) | |
| | (HPN321) (hGPCR21) - Homo | | | |
| | sapiens (Human), 346 aa. | | | |
| CAC69290 | Sequence 1 from Patent | 1 . . . 346 | 344/346 (99%) | 0.0 |
| | WO0159118 - Homo sapiens | 1 . . . 346 | 345/346 (99%) | |
| | (Human), 346 aa. | | | |
| Q95N03 | Cysteinyl leukotriene receptor 2 | 1 . . . 346 | 275/347 (79%) | e−158 |
| | (CysLTR2) - Sus scrofa (Pig), | 1 . . . 345 | 300/347 (86%) | |
| | 345 aa. | | | |
| Q8R528 | Cysteinyl leukotriene 2 receptor - | 17 . . . 324 | 226/308 (73%) | e−132 |
| | Mus musculus (Mouse), 309 aa. | 1 . . . 308 | 256/308 (82%) | |
| Q920A1 | Cysteinyl leukotriene receptor 2 | 17 . . . 324 | 224/308 (72%) | e−131 |
| | (CysLTR2) - Mus musculus | 1 . . . 308 | 255/308 (82%) | |
| | (Mouse), 309 aa. | | | |

PFam analysis predicts that the NOV6a protein contains the domains shown in the Table 6F.

TABLE 6F
Domain Analysis of NOV6a

| | | Identities/ | |
|---|---|---|---|
| | | Similarities | |
| Pfam | NOV6a Match | for the | Expect |
| Domain | Region | Matched Region | Value |
| 7tm_1 | 55 . . . 305 | 89/277 (32%) | 6.1e−54 |
| | | 185/277 (67%) | |
| TAS2R | 33 . . . 324 | 64/314 (20%) | 0.019 |
| | | 173/314 (55%) | |

### Example 7

The NOV7 clone was analyzed, and the nucleotide and encoded polypeptide sequences are shown in Table 7A.

TABLE 7A
NOV7 Sequence Analysis

| | | |
|---|---|---|
| NOV7a, CG54566-01 | SEQ ID NO: 25 | 1358 bp |
| DNA Sequence | ORF Start: ATG at 5 | ORF Stop: end of sequence |

AGTGATGGATGTTAACAGCAGCGGCCACCCGGACCTCTACGGGCGCCTCTGCTCTTTCCTCCTGCCGG
AGGTGGGGGGCAGGCTGCCCGACCTGAGCCCCGACGGTGGCGCCGAACCGGTCGCGGTCTCCGGGACG
CCGCATCTGCTGAGCGAGGTGACGGCCAGCCCGGCGCCCACCTGGGACGCAACCCCGGGCAATGCCTC
CGGCCGCGGGGAGCAAATCAATCAAGAAAGGGCCGAGAAAGTTGTGATCGGCTCTGTCCTGACGCTCA
TCTCTCTGTCTGCGATCGCGGGCAACTGCCTGGTGGTAATCTCTGTGTGCTTCGTCAAGAAGCTCCGC
CAGCCCTCCAACTACCTCATCGTGTCCATGGCGCTGGCCAACCTCTCGGTGGCCATGGCGGTCATGCC
CTTCATCAGTGTCACCGACCTCATCGGGGGCAAGTGGATCTTTGGACACTTTTTCTGTAACGTCTTCT
CCGTGAATGTCATGTGCTGCACGGCCTGGATCTTGACCTTGTACGTGATCAGCATCGACAGGGACCTT
GGGATCATGAAGCCTCTCACGTACCCTATGAGGCAGAAGGGGAAATGCATGACGAAGATGATTCTTTC
TGTCTGCCTTCTTTCCGCCTTTGTCACTTTACCTACCATTTTTGGTCGGGCTCAGAATGTAAACGATG
ATAAGGTGTGCTTGGTCAATCAAGACTTTGGCTACACGATTTACTCCCCGCATTTGGCAGCATTTATC
CCCATGTGCGTCATGCTTTTCATGTACTATCAGATTTACAAGGCCGCCAGGAAAAGCGCGGCCAAACA
CAGGTTACCTGGCTTCCCTCGAGTGGAGCCAGACAGCGTAGTCACCCTGAATGGCACAGTGAAGTTCC
AGGAGGTGGAAGAGTGTGCAAACCTTTCGAGACTCCTCAAGCATGAAAGGAAAAATATCTCCATCTTT
AAGCGGAAACAGAAAGCAGCGACTACCTTGGGGATCATCGTCTGGGCCTCCACCATGTGCTGGCCGCC
CTTTTTCCTCCTGACAGCCAGACCCTTCTGTCTATGGCACTGCCCTTCTGTCTATGGCACTGCCTGCA
GCTGCATCCCACTGTGGGTGGAGAGGATATTTCCATGGCTGGGCTATGCAAACTCTCTCATTAACCCT
TTTATTTATGCCTTCTTCAACTGGGACCTGAGGACCACCTATTGCAGCCGGCTCCAGTGCCAGTACCA
GAATATCAACCAGACACTCTCAGCTGCAGGCATGCATGAAGCCCTGAAGCTTGCTGAGAGGCCAGAGA
GACCTGAGTTTGTCCTACAAAACTCTGACTACTGTAGAAAAAAAAGTCATGATTCATGACTGAAAG

| | | | |
|---|---|---|---|
| NOV7a, CG54566-01 | SEQ ID NO: 26 | 448 aa | MW at 49974.9kD |
| Protein Sequence | | | |

MDVNSSGHPDLYGRLCSFLLPEVGGRLPDLSPDGGAEPVAVSGTPHLLSEVTASPAPTWDATPGNASG
RGEQINQERAEKVVIGSVLTLISLSAIAGNCLVVISVCFVKKLRQPSNYLIVSMALANLSVAMAVMPF
ISVTDLIGGKWIFGHFFCNVFSVNVMCCTAWILTLYVISIDRDLGIMKPLTYPMRQKGKCMTKMILSV
CLLSAFVTLPTIFGRAQNVNDDKVCLVNQDFGYTIYSPHLAAFIPMCVMLFMYYQIYKAARKSAAKHR
LPGFPRVEPDSVVTLNGTVKFQEVEECANLSRLLKHERKNISIFKRKQKAATTLGIIVWASTMCWPPF
FLLTARPFCLWHCPSVYGTACSCIPLWVERIFPWLGYANSLINPFIYAFFNWDLRTTYCSRLQCQYQN
INQTLSAAGMHEALKLAERPERPEFVLQNSDYCRKKSHDS

Further analysis of the NOV7a protein yielded the following properties shown in Table 7B.

TABLE 7B
Protein Sequence Properties NOV7a

| SignalP analysis: | No Known Signal Sequence Predicted | | | | |
|---|---|---|---|---|---|
| PSORT II analysis: | PSG: a new signal peptide prediction method | | | | |
| | N-region: length 10; pos. chg 0; neg. chg 2 | | | | |
| | H-region: length 3; peak value 0.00 | | | | |
| | PSG score: −4.40 | | | | |
| | GvH: von Heijne's method for signal seq. recognition | | | | |
| | GvH score (threshold: −2.1): −3.34 | | | | |
| | possible cleavage site: between 25 and 26 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 5 | | | | |
| | INTEGRAL | Likelihood = | −6.58 | Transmembrane | 92-108 |
| | INTEGRAL | Likelihood = | −4.99 | Transmembrane | 118-134 |
| | INTEGRAL | Likelihood = | −1.81 | Transmembrane | 160-176 |
| | INTEGRAL | Likelihood = | −5.84 | Transmembrane | 201-217 |
| | INTEGRAL | Likelihood = | −1.91 | Transmembrane | 244-260 |
| | PERIPHERAL | Likelihood = | 0.63 (at 326) | | |

| ALOM score: −6.58 (number of TMSs: 5) | | | |
|---|---|---|---|
| MTOP: Prediction of membrane topology (Hartmann et al.) | | | |
| Center position for calculation: 99 | | | |
| Charge difference: 3.0 C(3.0)-N(0.0) | | | |
| C > N: C-terminal side will be inside | | | |
| >>> membrane topology: type 3b | | | |
| MITDISC: discrimination of mitochondrial targeting seq | | | |
| R content: | 0 | Hyd Moment (75): | 3.84 |
| Hyd Moment (95): | 1.55 | G content: | 1 |
| D/E content: | 2 | S/T content: | 2 |

Score: −7.77
Gavel: prediction of cleavage sites for mitochondrial preseq
cleavage site motif not found
NUCDISC: discrimination of nuclear localization signals
pat4: none
pat7: none
bipartite: none
content of basic residues: 9.4%
NLS Score: −0.47
KDEL: ER retention motif in the C-terminus: none
ER Membrane Retention Signals:
KKXX-like motif in the C-terminus: KSHD
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
44.4%: endoplasmic reticulum
22.2%: vacuolar
11.1%: mitochondrial
11.1%: Golgi
11.1%: cytoplasmic
>> prediction for CG54566-01 is end (k = 9)

A search of the NOV7a protein against the Geneseq database, a proprietary database that contains sequences published in patents and patent publication, yielded several homologous proteins shown in Table 7C.

TABLE 7C
Geneseq Results for NOV7a

| | | NOV7a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE15653 | Human 5-hydroxytryptamine-7- | 1 . . . 448 | 448/448 (100%) | 0.0 |
| | receptor-like protein, NOV4 - | 1 . . . 448 | 448/448 (100%) | |
| | Homo sapiens, 448 aa. | | | |
| | [WO200194416-A2, 13 DEC. | | | |
| | 2001] | | | |
| ABP81770 | Human 5-HT7 receptor protein | 1 . . . 448 | 368/451 (81%) | 0.0 |
| | SEQ ID NO: 22 - Homo sapiens, | 2 . . . 445 | 396/451 (87%) | |
| | 445 aa. [WO200261087-A2, 08 | | | |
| | AUG. 2002] | | | |
| AAR54782 | Human brain serotonin receptor | 1 . . . 448 | 368/451 (81%) | 0.0 |
| | 5-HT4B protein - Homo sapiens, | 2 . . . 445 | 396/451 (87%) | |
| | 445 aa. [WO9409828-A, 11 | | | |
| | MAY 1994] | | | |
| ABB56329 | Non-endogenous human GPCR | 1 . . . 448 | 367/451 (81%) | 0.0 |
| | protein, SEQ ID NO: 451 - Homo | 2 . . . 445 | 395/451 (87%) | |
| | sapiens, 445 aa. [WO200177172- | | | |
| | A2, 18 OCT. 2001] | | | |
| AAR57200 | Rat 5HT6 receptor - Rattus rattus, | 1 . . . 448 | 357/454 (78%) | 0.0 |
| | 448 aa. [FR2699922-A, 01 JUL. | 2 . . . 445 | 389/454 (85%) | |
| | 1994] | | | |

In a BLAST search of public sequence databases, the NOV7a protein was found to have homology to the proteins shown in the BLASTP data in Table 7D.

TABLE 7D
Public BLASTP Results for NOV7a

| | | NOV7a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD32672 | Sequence 11 from Patent | 1 . . . 448 | 448/448 (100%) | 0.0 |
| | WO0194416 - Homo sapiens | 1 . . . 448 | 448/448 (100%) | |
| | (Human), 448 aa. | | | |
| AAH47526 | 5-hydroxytryptamine (serotonin) | 1 . . . 448 | 368/451 (81%) | 0.0 |
| | receptor 7 (adenylate cyclase- | 2 . . . 445 | 396/451 (87%) | |
| | coupled) - Homo sapiens | | | |
| | (Human), 445 aa. | | | |
| P34969 | 5-hydroxytryptamine 7 receptor | 1 . . . 436 | 357/439 (81%) | 0.0 |
| | (5-HT-7) (5-HT-X) (Serotonin | 2 . . . 433 | 385/439 (87%) | |
| | receptor) (5HT7) - Homo sapiens | | | |
| | (Human), 479 aa. | | | |
| P32305 | 5-hydroxytryptamine 7 receptor | 1 . . . 448 | 357/454 (78%) | 0.0 |
| | (5-HT-7) (5-HT-X) (Serotonin | 2 . . . 448 | 389/454 (85%) | |
| | receptor) (5HT7) (GPRFO) - | | | |
| | Rattus norvegicus (Rat), 448 aa. | | | |
| Q8SPH2 | Serotonin 5-hydroxytryptamine | 1 . . . 448 | 361/454 (79%) | 0.0 |
| | 7-a receptor - Sus scrofa (Pig), | 2 . . . 447 | 387/454 (84%) | |
| | 447 aa. | | | |

PFam analysis predicts that the NOV7a protein contains the domains shown in the Table 7E.

TABLE 7E
Domain Analysis of NOV7a

| | | Identities/ | |
|---|---|---|---|
| | | Similarities | |
| Pfam | NOV7a Match | for the | Expect |
| Domain | Region | Matched Region | Value |
| 7tm_1 | 97 . . . 387 | 76/315 (24%) | 3.8e−56 |
| | | 219/315 (70%) | |

### Example 8

The NOV8 clone was analyzed, and the nucleotide and encoded polypeptide sequences are shown in Table 8A.

TABLE 8A
NOV8 Sequence Analysis

| | | |
|---|---|---|
| NOV8a, CG55912-01 | SEQ ID NO: 27 | 1173 bp |
| DNA Sequence | ORF Start: ATG at 55 | ORF Stop: end of sequence |

AATATAGCCGGTCCTGTCCCCGTGTTAACTGGAGTGCTGAAGCGCTGGAACGAAATGCGGGGCCTCTG
GTGCGAGAAGGGGGTGCAGGTGCTGCTGACGACGGTGGGCGCCTTCGCCGCCTTCGGCCTCATGACCA
TCGCCATCAGCACTGACTACTGGCTCTACACGGGGCAACAAGAGCGAAAATCTGTCTCAAAAAATAAA
AGAAGTAAGAAGGACCCCGGCGGCCTCACGCACTCGGGCCTCTGGAGGATCTGCTGCCTGGAAGGGTT
GAAAAGAGGCGTCTGCGTGAAGATCAATCATTTCCCGGAGGACACGGACTACGACCACGACAGCGCGG
AGTATCTACTCCGTACGGTCCGGGCCTCCAGCATCTTCCCCATCCTTAGCGCCATCCTGCTGCTGCTC
GGGGGTGTGTGCGTGGCGGCCTCCCGCGTCTACAAGTCCAAGAGGAACATCATTCTGGGCGCAGGGAT
CCTGTTCGTGGCAGCAGGTCTGAGCAACATCATCGGCGTGATCGTGTACATCTCCGCCAACGCGGGCG
AGCCGGGCCGAAGCAGAGCCAAGAAAAACCACTACTCGTACGGCTGGTCCTTCTACTTCGGCGGGCTG
TCGTTCATCCTGGCCGAGGTGATAGGCGTGCTGGCCGTCAACATCTACATCGAGCCAGCCGAGGCGCA
CTGCCAGTCTCGGAGCGGGACCGCGGGGGGTCGTCCGGCTTCCTCACGCTGCACAACGCCTTCCCCAA
GGAGGCGGGCGGCGGCGTCACGGTCACGTTCACCCGGCCGCCCGCCCCGCCCGCGCCACGCCACCCCG
GCCAACACCAACTCCACGGACATCTCCATGTACACGCTCAGCCGCGACCCCTCCAAGGGCAGCCCCCA
TTCCAATGCCACCACCCCCACCCCCACTAGCCTCAAGGATAGGAAAAACTCATTTGTGTCCATAAAAA
TAAAGGTAAAAAAGAAAAAAAGAAATATATATATATATATATATACGCTCAACAGGAAAACCACGCCT
GTGTAGGGGCGCGGCGGGGGAGCCGAGGGGCGTGTCCGGGGCGCGTGCGGGCGCGCGTGCATCGAGGC
TGCCGGGGTCGGGGGCGCCCCCGCTTTCCCCCGTGAGCGCGCTGGAGACTGCTGGGCCCGCCCCACGC
CCACCCTCCCCGCCCCC

| | | | |
|---|---|---|---|
| NOV8a, CG55912-01 | SEQ ID NO: 28 | 323 aa | MW at 35342.3kD |
| Protein Sequence | | | |

MRGLWCEKGVQVLLTTVGAFAAFGLMTIAISTDYWLYTGQQERKSVSKNKRSKKDPGGLTHSGLWRIC
CLEGLKRGVCVKINHFPEDTDYDHDSAEYLLRTVRASSIFPILSAILLLLGGVCVAASRVYKSKRNII
LGAGILFVAAGLSNIIGVIVYISANAGEPGRSRAKKNHYSYGWSFYFGGLSFILAEVIGVLAVNIYIE
PAEAHCQSRSGTAGGRPASSRCTTPSPRRRAAASRSRSPGRPPRPRHATPANTNSTDISMYTLSRDPS
KGSPHSNATTPTPTSLKDRKNSFVSIKIKVKKKKRNIYIYIYTLNRKTTPV

Further analysis of the NOV8a protein yielded the following properties shown in Table 8B.

TABLE 8B
Protein Sequence Properties NOV8a

| SignalP analysis: | Cleavage site between residues 22 and 23 | | | | |
|---|---|---|---|---|---|
| PSORT II analysis: | PSG: a new signal peptide prediction method | | | | |
| | N-region: length 8; pos. chg 2; neg. chg 1 | | | | |
| | H-region: length 24; peak value 10.41 | | | | |
| | PSG score: 6.01 | | | | |
| | GvH: von Heijne's method for signal seq. recognition | | | | |
| | GvH score (threshold: −2.1): −3.71 | | | | |
| | possible cleavage site: between 24 and 25 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 4 | | | | |
| | INTEGRAL | Likelihood = | −3.93 | Transmembrane | 12-28 |
| | INTEGRAL | Likelihood = | −9.50 | Transmembrane | 107-123 |
| | INTEGRAL | Likelihood = | −6.48 | Transmembrane | 140-156 |
| | INTEGRAL | Likelihood = | −5.20 | Transmembrane | 183-199 |
| | PERIPHERAL | Likelihood = | 7.21 (at 63) | | |

| ALOM score: −9.50 (number of TMSs: 4) | | | |
|---|---|---|---|
| MTOP: Prediction of membrane topology (Hartmann et al.) | | | |
| Center position for calculation: 19 | | | |
| Charge difference: −2.0 C(0.0)-N(2.0) | | | |
| N >= C: N-terminal side will be inside | | | |
| >>> membrane topology: type 3a | | | |
| MITDISC: discrimination of mitochondrial targeting seq | | | |
| R content: | 1 | Hyd Moment (75): | 13.83 |
| Hyd Moment (95): | 3.82 | G content: | 4 |
| D/E content: | 2 | S/T content: | 5 |

```
Score: −5.73
Gavel: prediction of cleavage sites for mitochondrial preseq
R-2 motif at 12 MRG|LW
NUCDISC: discrimination of nuclear localization signals
pat4: PRRR (4) at 231
pat4: KKKK (5) at 303
pat4: KKKR (5) at 304
pat7: PSPRRRA (4) at 229
pat7: PRRRAAA (5) at 231
bipartite: RKNSFVSIKIKVKKKKR at 291
content of basic residues: 14.9%
NLS Score: 2.00
KDEL: ER retention motif in the C-terminus: none
ER Membrane Retention Signals:
XXRR-like motif in the N-terminus: RGLW
KKXX-like motif in the C-terminus: KTTP
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 76.7
COIL: Lupas's algorithm to detect coiled-coil regions
total: 0 residues
Final Results (k = 9/23):
39.1%: endoplasmic reticulum
30.4%: nuclear
17.4%: mitochondrial
  4.3%: vesicles of secretory system
  4.3%: cytoplasmic
  4.3%: peroxisomal
>> prediction for CG55912-01 is end (k = 23)
```

A search of the NOV8a protein against the Geneseq database, a proprietary database that contains sequences published in patents and patent publication, yielded several homologous proteins shown in Table 8C.

TABLE 8C
Geneseq Results for NOV8a

| | | NOV8a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY70462 | Human membrane channel | 7 . . . 323 | 183/327 (55%) | 6e−93 |
| | protein-12 (MECHP-12) - Homo | 5 . . . 323 | 233/327 (70%) | |
| | sapiens, 323 aa. [WO200012711- | | | |
| | A2, 09 MAR. 2000] | | | |
| ABB11805 | Human voltage gated Ca channel | 7 . . . 323 | 183/327 (55%) | 1e−92 |
| | subunit homologue, SEQ ID | 7 . . . 325 | 233/327 (70%) | |
| | NO: 2175 - Homo sapiens, 325 aa. | | | |
| | [WO200157188-A2, 09 AUG. | | | |
| | 2001] | | | |
| AAY84376 | A human voltage-gated calcium | 7 . . . 323 | 183/327 (55%) | 1e−92 |
| | channel designated | 5 . . . 323 | 233/327 (70%) | |
| | CACNGLIKE1 - Homo sapiens, | | | |
| | 323 aa. [WO200014223-A1, 16 | | | |
| | MAR. 2000] | | | |
| AAU97153 | Mouse neuronal voltage-gated | 7 . . . 323 | 183/327 (55%) | 2e−92 |
| | calcium channel gamma subunit | 5 . . . 323 | 232/327 (69%) | |
| | Cacng2 - Murinae gen. sp, 323 aa. | | | |
| | [US6365337-B1, 02 APR. 2002] | | | |
| AAY84374 | A human a neuronal voltage-gated | 6 . . . 323 | 173/324 (53%) | 1e−85 |
| | calcium chanel polypeptide - | 4 . . . 315 | 217/324 (66%) | |
| | Homo sapiens, 315 aa. | | | |
| | [WO200014225-A1, 16 MAR. | | | |
| | 2000] | | | |

In a BLAST search of public sequence databases, the NOV8a protein was found to have homology to the proteins shown in the BLASTP data in Table 8D.

TABLE 8D
Public BLASTP Results for NOV8a

| | | NOV8a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8VHW5 | Voltage-dependent calcium channel | 2 . . . 275 | 226/309 (73%) | e−113 |
| | gamma-8 subunit (Neuronal | 11 . . . 317 | 234/309 (75%) | |
| | voltage-gated calcium channel | | | |
| | gamma-8 subunit) - Rattus | | | |
| | norvegicus (Rat), 421 aa. | | | |
| Q8WXS5 | Voltage-dependent calcium channel | 2 . . . 275 | 229/310 (73%) | e−113 |
| | gamma-8 subunit (Neuronal | 11 . . . 317 | 236/310 (75%) | |
| | voltage-gated calcium channel | | | |
| | gamma-8 subunit) - Homo sapiens | | | |
| | (Human), 425 aa. | | | |
| Q8VHW2 | Voltage-dependent calcium channel | 2 . . . 275 | 225/309 (72%) | e−113 |
| | gamma-8 subunit (Neuronal | 11 . . . 317 | 234/309 (74%) | |
| | voltage-gated calcium channel | | | |
| | gamma-8 subunit) - Mus musculus | | | |
| | (Mouse), 423 aa. | | | |
| Q9Y698 | Voltage-dependent calcium channel | 7 . . . 323 | 183/327 (55%) | 4e−92 |
| | gamma-2 subunit (Neuronal | 5 . . . 323 | 233/327 (70%) | |
| | voltage-gated calcium channel | | | |
| | gamma-2 subunit) - Homo sapiens | | | |
| | (Human), 323 aa. | | | |
| O88602 | Voltage-dependent calcium channel | 7 . . . 323 | 183/327 (55%) | 5e−92 |
| | gamma-2 subunit (Neuronal | 5 . . . 323 | 232/327 (69%) | |
| | voltage-gated calcium channel | | | |
| | gamma-2 subunit) (Stargazin) - | | | |
| | Mus musculus (Mouse), 323 aa. | | | |

PFam analysis predicts that the NOV8a protein contains the domains shown in the Table 8E.

TABLE 8E
Domain Analysis of NOV8a

| | | Identities/ | |
|---|---|---|---|
| | | Similarities for | |
| Pfam | NOV8a Match | the Matched | Expect |
| Domain | Region | Region | Value |
| PMP22_Claudin | 8 . . . 198 | 51/198 (26%) | 7.6e−53 |
| | | 161/198 (81%) | |

### Example 9

The NOV9 clone was analyzed, and the nucleotide and encoded polypeptide sequences are shown in Table 9A.

TABLE 9A
NOV9 Sequence Analysis

| | | |
|---|---|---|
| NOV9a, CG56001-01 | SEQ ID NO: 29 | 1192 bp |
| DNA Sequence | ORF Start: ATG at 69 | ORF Stop: end of sequence |

TGCTGAGGGTGCATTTATGTTTCAGAACCACCGGGAGGAACTGGGCCATTCTAACACCCGTTGCTACC
ATGCTGGCCACCCGCCTCTCCAGACCCCTGTCACGGCTCCCAGGAAAAACCCTAAGTGCCTGTGATAG
AGAAAATGGAGCAAGGCGCCCACTATTGCTTGGTTCTACTTCCTTTATCCCGATTGGCCGTCGGACTT
ATGCCAGTGCGGCGGAGCCGGTGAGTGGAAAAGCTGTCCTGGTCACAGGCTGTGACTCTGGATTTGGG
TTCTCATTGGCCAAGCATCTGCATTCAAAAGGCTTCCTTGTGTTTGCTGGCTGCTTGATGAAGGACAA
AGGCCATGATGGGGTCAAGGAGCTGGACAGCCTAAACAGTGACCGATTGAGAACCGTCCAGCTCAATG
TCTGCAGCAGCGAAGAGGTGGAGAAAGTGGTGGAGATTGTCCGCTCGAGCCTGAAGGACCCTGAGAAA
GGTATGTGGGGCCTCGTTAACAATGCCGGCATCTCAACGTTCGGGGAGGTGGAGTTCACCAGCCTGGA
GACCTACAAGCAGGTGGCAGAAGTGAACCTTTGGGGCACAGTGCGGATGACGAAATCCTTTCTCCCCC
TCATCCGAAGGGCCAAAGGTCGCGTCGTCAATATCAGCAGCATGCTGGGCCGCATGGCCAACCCGGCC
CGCTCCCCGTACTGCATCACCAAGTTCGGGGTAGAGGCTTTCTCGGACTGCCTGCGCTATGAGATGTA
CCCCCTGGGCGTGAAGGTCAGCGTGGTGGAGCCCGGCAACTTCATCGCTGCCACCAGCCTTTACAGCC
CTGAGAGCATTCAGGCCATCGCCAAGAAGATGTGGGAGGAGCTGCCTGAGGTCGTGCGCAAGGACTAC
GGCAAGAAGTACTTTGATGAAAAGATCGCCAAGATGGAGACCTACTGCAGCAGTGGCTCCACAGACAC
GTCCCCTGTCATCGATGCTGTCACACACGCCCTGACCGCCACCACCCCCTACACCCGCTACCACCCCA
TGGACTACTACTGGTGGCTGCGAATGCAGATCATGACCCACTTGCCTGGAGCCATCTCCGACATGATC
TACATCCGCTGAAGAGTCTCGCTGTGGCCTCTGTCAGGGATCCCTGGTGGAAGGGGAGGGGAGGGAGG
AACCCATATAGTCAACTCTTGATTATCCACGTGTGG

| | | | |
|---|---|---|---|
| NOV9a, CG56001-01 | SEQ ID NO: 30 | 343 aa | MW at 38156.7kD |
| Protein Sequence | | | |

MLATRLSRPLSRLPGKTLSACDRENGARRPLLLGSTSFIPIGRRTYASAAEPVSGKAVLVTGCDSGFG
FSLAKHLHSKGFLVFAGCLMKDKGHDGVKELDSLNSDRLRTVQLNVCSSEEVEKVVEIVRSSLKDPEK
GMWGLVNNAGISTFGEVEFTSLETYKQVAEVNLWGTVRMTKSFLPLIRRAKGRVVNISSMLGRMANPA
RSPYCITKFGVEAFSDCLRYEMYPLGVKVSVVEPGNFIAATSLYSPESIQAIAKKMWEELPEVVRKDY
GKKYFDEKIAKMETYCSSGSTDTSPVIDAVTHALTATTPYTRYHPMDYYWWLRMQIMTHLPGAISDMI
YIR

| | | |
|---|---|---|
| NOV9b, CG56001-02 | SEQ ID NO: 31 | 1166 bp |
| DNA Sequence | ORF Start: ATG at 69 | ORF Stop: end of sequence |

TGCTGAGGGTGCATTTATGTTTCAGAACCACCGGGAGGAACTGGGCCATTCTAACACCCGTTGCTACC
ATGCTGGCCACCCGCCTCTCCAGACCCCTGTCACGGCTCCCAGGAAAAACCCTAAGTGCCTGTGATAG
AGAAAATGGAGCAAGACGCCCACTATTGCTTGGTTCTACTTCCTTTATCCCGATTGGCCGTCGGACTT
ATGCCAGTGCGGCGGAGCCGGTTGGCAGCAAAGCTGTCCTGGTCACAGGCTGTGACTCTGGATTTGGG
TTCTCATTGGCCAAGCATCTGCATTCAAAAGGCTTCCTTGTGTTTGCTGGCTGCTTGATGAAGGACAA
AGGCCATGATGGGGTCAAGGAGCTGGACAGCCTAAACAGTGACCGATTGAGAACCGTCCAGCTCAATG
TCTGCAGCAGCGAAGAGGTGGAGAAAGTGGTGGAGATTGTCCGCTCGAGCCTGAAGGACCCTGAGAAA
GGCATGTGGGGCCTCGTTAACAATGCCGGCATCTCAACGTTCGGGGAGGTGGAGTTCACCAGCCTGGA
GACCTACAAGCAGGTGGCAGAAGTGAACCTTTGGGGCACAGTGCGGATGACGAAATCCTTTCTCCCCC
TCATCCGAAGGGCCAAAGGCCGCGTCGTCAATATCAGCAGCATGCTGGGCCGCATGGCCAACCCGGCC
CGCTCCCCGTACTGCATCACCAAGTTCGGGGTAGAGGCTTTCTCGGACTGCCTGCGCTATGAGATGTA
CCCCCTGGGCGTGAAGGTCAGCGTGGTGGAGCCCGGCAACTTCATCGCTGCCACCAGCCTTTACAGCC
CTGAGAGCATTCAGGCCATCGCCAAGAAGATGTGGGAGGAGCTGCCTGAGGTCGTGCGCAAGGACTAC
GGCAAGAAGTACTTTGATGAAAAGATCGCCAAGATGGAGACCTACTGCAGCAGTGGCTCCACAGACAC
GTCCCCTGTCATCGATGCTGTCACACACGCCCTGACCGCCACCACCCCCTACACCCGCTACCACCCCA
TGGACTACTACTGGTGGCTGCGAATGCAGATCATGACCCACTTGCCTGGAGCCATCTCCGACATGATC
TACATCCGCTGAAGAGTCTCGCTGTGGCCTCTGTCAGGGATTCCTGGTGGAAGGGGAGGGGAGGGAGG
AACCCATATA

| | | | |
|---|---|---|---|
| NOV9b, CG56001-02 | SEQ ID NO: 32 | 343 aa | MW at 38156.7kD |
| Protein Sequence | | | |

MLATRLSRPLSRLPGKTLSACDRENGARRPLLLGSTSFIPIGRRTYASAAEPVGSKAVLVTGCDSGFG
FSLAKHLHSKGFLVFAGCLMKDKGHDGVKELDSLNSDRLRTVQLNVCSSEEVEKVVEIVRSSLKDPEK
GMWGLVNNAGISTFGEVEFTSLETYKQVAEVNLWGTVRMTKSFLPLIRRAKGRVVNISSMLGRMANPA
RSPYCITKFGVEAFSDCLRYEMYPLGVKVSVVEPGNFIAATSLYSPESIQAIAKKMWEELPEVVRKDY
GKKYFDEKIAKMETYCSSGSTDTSPVIDAVTHALTATTPYTRYHPMDYYWWLRMQIMTHLPGAISDMI
YIR

A ClustalW comparison of the above protein sequences yields the following sequence alignment shown in Table 9B.

TABLE 9B
Comparison of the NOV9 protein sequences.

| | |
|---|---|
| NOV9a | MLATRLSRPLSRLPGKTLSACDRENGARRPLLLGSTSFIPIGRRTYASAAEPVSGKAVLV |
| NOV9b | MLATRLSRPLSRLPGKTLSACDRENGARRPLLLGSTSFIPIGRRTYASAAEPVGSKAVLV |
| NOV9a | TGCDSGFGFSLAKHLHSKGFLVFAGCLMKDKGHDGVKELDSLNSDRLRTVQLNVCSSEEV |
| NOV9b | TGCDSGFGFSLAKHLHSKGFLVFAGCLMKDKGHDGVKELDSLNSDRLRTVQLNVCSSEEV |
| NOV9a | EKVVEIVRSSLKDPEKGMWGLVNNAGISTFGEVEFTSLETYKQVAEVNLWGTVRMTKSFL |
| NOV9b | EKVVEIVRSSLKDPEKGMWGLVNNAGISTFGEVEFTSLETYKQVAEVNLWGTVRMTKSFL |
| NOV9a | PLIRRAKGRVVNISSMLGRMANPARSPYCITKFGVEAFSDCLRYEMYPLGVKVSVVEPGN |
| NOV9b | PLIRRAKGRVVNISSMLGRMANPARSPYCITKFGVEAFSDCLRYEMYPLGVKVSVVEPGN |
| NOV9a | FIAATSLYSPESIQAIAKKMWEELPEVVRKDYGKKYFDEKIAKMETYCSSGSTDTSPVID |
| NOV9b | FIAATSLYSPESIQAIAKKMWEELPEVVRKDYGKKYFDEKIAKMETYCSSGSTDTSPVID |
| NOV9a | AVTHALTATTPYTRYHPMDYYWWLRMQIMTHLPGAISDMIYIR |
| NOV9b | AVTHALTATTPYTRYHPMDYYWWLRMQIMTHLPGAISDMIYIR |
| NOV9a | (SEQ ID NO: 30) |
| NOV9b | (SEQ ID NO: 32) |

Further analysis of the NOV9a protein yielded the following properties shown in Table 9C.

TABLE 9C
Protein Sequence Properties NOV9a

| SignalP analysis: | Cleavage site between residues 13 and 14 | | | |
|---|---|---|---|---|
| PSORT II analysis: | PSG: a new signal peptide prediction method | | | |
| | N-region: length 8; pos. chg 2; neg. chg 0 | | | |
| | H-region: length 3; peak value −7.47 | | | |
| | PSG score: −11.88 | | | |
| | GvH: von Heijne's method for signal seq. recognition | | | |
| | GvH score (threshold: −2.1): −5.98 | | | |
| | possible cleavage site: between 43 and 44 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | 5.36 (at 56) Likelihood = PERIPHERAL | | | |
| | ALOM score: 5.36 (number of TMSs: 0) | | | |
| | MITDISC: discrimination of mitochondrial targeting seq | | | |
| | R content: | 3 | Hyd Moment (75): | 8.06 |
| | Hyd Moment (95): | 10.84 | G content: | 1 |
| | D/E content: | 1 | S/T content: | 5 |

Score: −0.40
Gavel: prediction of cleavage sites for mitochondrial preseq
R-3 motif at 47 RRTY|A
NUCDISC: discrimination of nuclear localization signals
pat4: none
pat7: PLIRRAK (3) at 181
bipartite: none
content of basic residues: 12.8%
NLS Score: −0.22
KDEL: ER retention motif in the C-terminus: none
ER Membrane Retention Signals:
XXRR-like motif in the N-terminus: LATR
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
78.3%: mitochondrial
13.0%: cytoplasmic
8.7%: nuclear
>> prediction for CG56001-01 is mit (k = 23)

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP53584 | Human NOV14a protein SEQ ID | 1 . . . 343 | 343/343 (100%) | 0.0 |
| | NO: 32 - Homo sapiens, 343 aa. | 1 . . . 343 | 343/343 (100%) | |
| | [WO200262999-A2, 15 AUG. | | | |
| | 2002] | | | |
| ABP53584 | Human NOV14a protein SEQ ID | 1 . . . 343 | 343/343 (100%) | 0.0 |
| | NO: 32 - Homo sapiens, 343 aa. | 1 . . . 343 | 343/343 (100%) | |
| | [WO200262999-A2, 15 AUG. | | | |
| | 2002] | | | |
| ABP53585 | Human NOV14b protein SEQ | 1 . . . 343 | 341/343 (99%) | 0.0 |
| | ID NO: 34 - Homo sapiens, 343 | 1 . . . 343 | 341/343 (99%) | |
| | aa. [WO200262999-A2, 15 | | | |
| | AUG. 2002] | | | |
| ABP53585 | Human NOV14b protein SEQ | 1 . . . 343 | 341/343 (99%) | 0.0 |
| | ID NO: 34 - Homo sapiens, 343 | 1 . . . 343 | 341/343 (99%) | |
| | aa. [WO200262999-A2, 15 | | | |
| | AUG. 2002] | | | |
| AAM78804 | Human protein SEQ ID NO | 1 . . . 343 | 341/343 (99%) | 0.0 |
| | 1466 - Homo sapiens, 343 aa. | 1 . . . 343 | 341/343 (99%) | |
| | [WO200157190-A2, 09 AUG. | | | |
| | 2001] | | | |

In a BLAST search of public sequence databases, the NOV9a protein was found to have homology to the proteins shown in the BLASTP data in Table 9E.

TABLE 9E
Public BLASTP Results for NOV9a

| | | NOV9a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q02338 | D-beta-hydroxybutyrate | 1 . . . 343 | 341/343 (99%) | 0.0 |
| | dehydrogenase, mitochondrial | 1 . . . 343 | 341/343 (99%) | |
| | precursor (EC 1.1.1.30) (BDH) (3- | | | |
| | hydroxybutyrate dehydrogenase) - | | | |
| | Homo sapiens (Human), 343 aa. | | | |
| A42845 | 3-hydroxybutyrate dehydrogenase | 11 . . . 343 | 319/333 (95%) | 0.0 |
| | (EC 1.1.1.30) - human, 343 aa | 11 . . . 343 | 321/333 (95%) | |
| | (fragment). | | | |
| P29147 | D-beta-hydroxybutyrate | 1 . . . 342 | 297/342 (86%) | e−174 |
| | dehydrogenase, mitochondrial | 2 . . . 343 | 313/342 (90%) | |
| | precursor (EC 1.1.1.30) (BDH) (3- | | | |
| | hydroxybutyrate dehydrogenase) - | | | |
| | Rattus norvegicus (Rat), 344 aa. | | | |
| AAH43683 | Hypothetical protein - Mus | 1 . . . 342 | 297/342 (86%) | e−174 |
| | musculus (Mouse), 343 aa. | 1 . . . 342 | 313/342 (90%) | |
| Q8BK53 | D-beta-hydroxybutyrate | 1 . . . 342 | 295/342 (86%) | e−173 |
| | dehydrogenase - Mus musculus | 1 . . . 342 | 312/342 (90%) | |
| | (Mouse), 343 aa. | | | |

PFam analysis predicts that the NOV9a protein contains the domains shown in the Table 9F.

TABLE 9F
Domain Analysis of NOV9a

| | | Identities/ | |
|---|---|---|---|
| | | Similarities for | |
| Pfam | NOV9a Match | the Matched | Expect |
| Domain | Region | Region | Value |
| adh_short | 54 . . . 336 | 80/291 (27%) | 7.9e−79 |
| | | 235/291 (81%) | |

### Example 10

The NOV10 clone was analyzed, and the nucleotide and encoded polypeptide sequences are shown in Table 10A.

TABLE 10A
NOV10 Sequence Analysis

| | | |
|---|---|---|
| NOV10a, CG56151-01 | SEQ ID NO: 33 | 3168 bp |
| DNA Sequence | ORF Start: ATG at 39 | ORF Stop: end of sequence |

CACAAGACCTGGAATTGACAGGACTCCCAACTAGTACAATGACAGAAGATAAGGTCACTGGGACCCTG
GTTTTCACTGTCATCACTGCTGTGCTGGGTTCCTTCCAGTTTGGATATGACATTGGTGTGATCAATGC
ACCTCAACAGGTAATAATATCTCACTATAGACATGTTTTGGGTGTTCCACTGGATGACCGAAAAGCTA
TCAACAACTATGTTATCAACAGTACAGATGAACTGCCCACAATCTCATACTCAATGAACCCAAAACCA
ACCCCTTGGGCTGAGGAAGAGACTGTGGCAGCTGCTCAACTAATCACCATGCTCTGGTCCCTGTCTGT
ATCCAGCTTTGCAGTTGGTGGAATGACTGCATCATTCTTTGGTGGGTGGCTTGGGGACACACTTGGAA
GAATCAAAGCCATGTTAGTAGCAAACATTCTGTCATTAGTTGGAGCTCTCTTGATGGGGTTTTCAAAA
TTGGGACCATCTCATATACTTATAATTGCTGGAAGAAGCATATCAGGACTATATTGTGGGCTAATTTC
AGGCCTGGTTCCTATGTATATCGGTGAAATTGCTCCAACCGCTCTCAGGGGAGCACTTGGCACTTTTC
ATCAGCTGGCCATCGTCACGGGCATTCTTATTAGTCAGATTATTGGTCTTGAATTTATCTTGGGCAAT
TATGATCTGTGGCACATCCTGCTTGGCCTGTCTGGTGTGCGAGCCATCCTTCAGTCTCTGCTACTCTT
TTTCTGTCCAGAAAGCCCCAGATACCTTTACATCAAGTTAGATGAGGAAGTCAAAGCAAAACAAAGCT
TGAAAAGACTCAGAGGATATGATGATGTCACCAAAGATATTAATGAAATGAGAAAAGAAAGAGAAGAA
GCATCGAGTGAGCAGAAAGTCTCTATAATTCAGCTCTTCACCAATTCCAGCTACCGACAGCCTATTCT
AGTGGCACTGATGCTGCATGTGGCTCAGCAATTTTCCGGAATCAATGGCATTTTTTACTACTCAACCA
GCATTTTTCAGACGGCTGGTATCAGCAAACCTGTTTATGCAACCATTGGAGTTGGCGCTGTAAACATG
GTTTTCACTGCTGTCTCTGTATTCCTTGTGGAGAAGGCAGGGCGACGTTCTCTCTTTCTAATTGGAAT
GAGTGGGATGTTTGTTTGTGCCATCTTCATGTCAGTGGGACTTGTGCTGCTGAATAAGTTCTCTTGGA
TGAGTTATGTGAGCATGATAGCCATCTTCCTCTTTGTCAGCTTCTTTGAAATTGGGCCAGGCCCGATC
CCCTGGTTCATGGTGGCTGAGTTTTTCAGTCAAGGACCACGTCCTGCTGCTTTAGCAATAGCTGCATT
CAGCAATTGGACCTGCAATTTCATTGTAGCTCTGTGTTTCCAGTACATTGCGGACTTCTGTGGACCTT
ATGTGTTTTTCCTCTTTGCTGGAGTGCTCCTGGCCTTTACCCTGTTCACATTTTTTAAAGTTCCAGAA
ACCAAAGGAAAGTCTTTTGAGGAAATTGCTGCAGAATTCCAAAAGAAGAGTGGCTCAGCCCACAGGCC
AAAAGCTGCTGTAGAAATGAAATTCCTAGGAGCTACAGAGACTGTGTAAAAAAAAACCCTGCTTTTT
GACATGAACAGAAACAATAAGGGAACCGTCTGTTTTTAAATGATGATTCCTTGAGCATTTTATATCCA
CATCTTTAAGTATTGTTTTATTTTTATGTGCTCTCATCAGAAATGTCATCAAATATTACCAAAAAAGT
ATTTTTTTAAGTTAGAGAATATATTTTTGATGGTAAGACTGTAATTAAGTAAACCAAAAAGGCTAGTT
TATTTTGTTACACTAAAGGGCAGGTGGTTCTAATATTTTTAGCTCTGTTCTTTATAACAAGGTTCTTC
TAAAATTGAAGAGATTTCAACATATCATTTTTTTAACACATAACTAGAAACCTGAGGATGCAACAAAT
ATTTATATATTTGAATATCATTAAATTGGAATTTTCTTACCCATATATCTTATGTTAAAGGAGATATG
GCTAGTGGCAATAAGTTCCATGTTAAAATAGACAACTCTTCCATTTATTGCACTCAGCTTTTTTCTTG
AGTACTAGAATTTGTATTTTGCTTAAAATTTTACTTTTGTTCTGTATTTTCATGTGGAATGGATTATA
GAGTATACTAAAAAATGTCTATAGAGAAAAACTTTCATTTTTGGTAGGCTTATCAAAATCTTTCAGCA
CTCAGAAAAGAAAACCATTTTAGTTCCTTTATTTAATGGCCAAATGGTTTTTGCAAGATTTAACACTA
AAAAGGTTTCACCTGATCATATAGCGTGGGTTATCAGTTAACATTAACATCTATTATAAAACCATGTT
GATTCCCTTCTGGTACAATCCTTTGAGTTATAGTTTGCTTTGCTTTTTAATTGAGGACAGCCTGGTTT
TCACATACACTCAAACAATCATGAGTCAGACATTTGGTATATTACCTCAAATTCCTAATAAGTTTGAT
CAAATCTAATGTAAGAAAATTTGAAGTAAAGGATTGATCACTTTGTTAAAAATATTTTCTGAATTATT
ATGTCTCAAAATAAGTTGAAAAGGTAGGGTTTGAGGATTCCTGAGTGTGGGCTTCTGAAACTTCATAA
ATGTTCAGCTTCAGACTTTTATCAAAATCCCTATTTAATTTTCCTGGAAAGACTGATTGTTTTATGGT
GTGTTCCTAACATAAAATAATCGTCTCCTTTGACATTTCCTTCTTTGTCTTAGCTGTATACAGATTCT
AGCCAAACTATTCTATGGCCATTACTAACACGCATTGTACACTATCTATCTGCCTTTACCTACATAGG
CAAATTGGAAATACACAGATGATTAAACAGACTTTAGCTTACAGTCAATTTTACAATTATGGAAATAT
AGTTCTGATGGGTCCCAAAAGCTTAGCAGGGTGCTAACGTATCTCTAGGCTGTTTTCTCCACCAACTG
GAGCACTGATCAATCCTTCTTATGTTTGCTTTAATGTGTATTGAAGAAAAGCACTTTTTAAAAAGTAC
TCTTTAAGAGTGAAATAATTAAAAACCACTGAACATTTGCTTTGTTTTCTAAAGTTGTTCACATATAT
GTAATTTAGCAGTCCAAAGAACAAGAAATTGTTTCTTTTC

| | | | |
|---|---|---|---|
| NOV10a, CG56151-01 | SEQ ID NO: 34 | 524 aa | MW at 57488.8kD |
| Protein Sequence | | | |

MTEDKVTGTLVFTVITAVLGSFQFGYDIGVINAPQQVIISHYRHVLGVPLDDRKAINNYVINSTDELP
TISYSMNPKPTPWAEEETVAAAQLITMLWSLSVSSFAVGGMTASFFGGWLGDTLGRIKAMLVANILSL
VGALLMGFSKLGPSHILIIAGRSISGLYCGLISGLVPMYIGEIAPTALRGALGTFHQLAIVTGILISQ
IIGLEFILGNYDLWHILLGLSGVRAILQSLLLFFCPESPRYLYIKLDEEVKAKQSLKRLRGYDDVTKD
INEMRKEREEASSEQKVSIIQLFTNSSYRQPILVALMLHVAQQFSGINGIFYYSTSIFQTAGISKPVY
ATIGVGAVNMVFTAVSVFLVEKAGRRSLFLIGMSGMFVCAIFMSVGLVLLNKFSWMSYVSMIAIFLFV
SFFEIGPGPIPWFMVAEFFSQGPRPAALAIAAFSNWTCNFIVALCFQYIADFCGPYVFFLFAGVLLAF
TLFTFFKVPETKGKSFEEIAAEFQKKSGSAHRPKAAVEMKFLGATETV

| | | |
|---|---|---|
| NOV10b, 246837923 | SEQ ID NO: 35 | 1587 bp |
| DNA Sequence | ORF Start: at 1 | ORF Stop: end of sequence |

GGATCCACCATGACAGAAGATAAGGTCACTGGGACCCTGGTTTTCACTGTCATCACTGCTGTGCTGGG
TTCCTTCCAGTTTGGATATGACATTGGTGTGATCAATGCACCTCAACAGGTAATAATATCTCACTATA
GACATGTTTTGGGTGTTCCACTGGATGACCGAAAAGCTATCAACAACTATGTTATCAACAGTACAGAT
GAACTGCCCACAATCTCATACTCAATGAACCCAAAACCAACCCCTTGGGCTGAGGAAGAGACTGTGGC
AGCTGCTCAACTAATCACCATGCTCTGGTCCCTGTCTGTATCCAGCTTTGCAGTTGGTGGAATGATTG
CATCATTCTTTGGTGGGTGGCTTGGGGACACACTTGGAAGAATCAAAGCCATGTTAGTAGCAAACATT
CTGTCATTAGTTGGAGCTCTCTTGATGGGGTTTTCAAAATTGGGACCATCTCATATACTTATAATTGC
TGGAAGAAGCATATCAGGACTATATTGTGGGCTAATTTCAGGCCTGGTTCCTATGTATATCGGTGAAA
TTGCTCCAACCGCTCTCAGGGGAGCACTTGGCACTTTTCATCAGCTGGCCATCGTCACGGGCATTCTT
ATTAGTCAGATTATTGGTCTTGAATTTATCTTGGGCAATTATGATCTGTGGCACATCCTGCTTGGCCT
GTCTGGTGTGCGAGCCATCCTTCAGTCTCTGCTACTCTTTTTCTGTCCAGAAAGCCCCAGATACCTTT
ACATCAAGTTAGATGAGGAAGTCAAAGCAAAACAAATCTTGAAAAGACTCAGAGGATATGATGATGTC
ACCAAAGATATTAATGAAATGAGAAAAGAAAGAGAAGAAGCATCGAGTGAGCAGAAAGTCTCTATAAT
TCAGCTCTTCACCAATTCCAGCTACCGACAGCCTATTCTAGTGGCACTGATGCTGCATGTGGCTCAGC
AATTTTCCGGAATCAATGGCATTTTTTACTACTCAACCAGCATTTTTCAGACGGCTGGTATCAGCAAA
CCTGTTTATGCAACCATTGGAGTTGGCGCTGTAAACATGGTTTTCACTGCTGTCTCTGTATTCCTTGT
GGAGAAGGCAGGGCGACGTTCTCTCTTTCTAATTGGAATGAGTGGGATGTTTGTTTGTGCCATCTTCA
TGTCAGTGGGACTTGTGCTGCTGAATAAGTTCTCTTGGATGAGTTATGTGAGCATGATAGCCATCTTC
CTCTTTGTCAGCTTCTTTGAAATTGGGCCAGGCCCGATCCCCTGGTTCATGGTGGCTGAGTTTTTCAG
TCAAGGACCACGTCCTGCTGCTTTAGCAATAGCTGCATTCAGCAATTGGACCTGCAATTTCATTGTAG
CTCTGTGTTTCCAGTACATTGCGGACTTCTGTGGACCTTATGTGTTTTTCCTCTTTGCTGGAGTGCTC
CTGGCCTTTACCCTGTTTACATTTTTTAAAGTTCCAGAAACCAAAGGAAAGTCTTTTGAGGAAATTGC
TGCAGAATTCCAAAAGAAGAGTGGCTCAGCCCACAGGCCAAAAGCTGCTGTAGAAATGAAATTCCTAG
GAGCTACAGAGACTGTGCTCGAG

| | | | |
|---|---|---|---|
| NOV10b, 246837923 | SEQ ID NO: 36 | 529 aa | MW at 58014.4kD |
| Protein Sequence | | | |

GSTMTEDKVTGTLVFTVITAVLGSFQFGYDIGVINAPQQVIISHYRHVLGVPLDDRKAINNYVINSTD
ELPTISYSMNPKPTPWAEEETVAAAQLITMLWSLSVSSFAVGGMIASFFGGWLGDTLGRIKAMLVANI
LSLVGALLMGFSKLGPSHILIIAGRSISGLYCGLISGLVPMYIGEIAPTALRGALGTFHQLAIVTGIL
ISQIIGLEFILGNYDLWHILLGLSGVRAILQSLLLFFCPESPRYLYIKLDEEVKAKQILKRLRGYDDV
TKDINEMRKEREEASSEQKVSIIQLFTNSSYRQPILVALMLHVAQQFSGINGIFYYSTSIFQTAGISK
PVYATIGVGAVNMVFTAVSVFLVEKAGRRSLFLIGMSGMFVCAIFMSVGLVLLNKFSWMSYVSMIAIF
LFVSFFEIGPGPIPWFMVAEFFSQGPRPAALAIAAFSNWTCNFIVALCFQYIADFCGPYVFFLFAGVL
LAFTLFTFFKVPETKGKSFEEIAAEFQKKSGSAHRPKAAVEMKFLGATETVLE

| | | |
|---|---|---|
| NOV10c, 246837941 | SEQ ID NO: 37 | 1587 bp |
| DNA Sequence | ORF Start: at 1 | ORF Stop: end of sequence |

GGATCCACCATGACAGAAGATAAGGTCACTGGGACCCTGGTTTTCACTGTCATCACTGCTGTGCTGGG
TTCCTTCCAGTTTGGATATGACATTGGTGTGATCAATGCACCTCAACAGGTAATAATATCTCACTATA
GACATGTTTTGGGTGTTCCACTGGATGACCGAAAAGCTATCAACAACTATGTTATCAACAGTACAGAT
GAACTGCCCACAATCTCATACTCAATGAACCCAAAACCAACCCCTTGGGCTGAGGAAGAGACTGTGGC
AGCTGCTCAACTAATCACCATGCTCTGGTCCCTGTCTGTATCCAGCTTTGCAGTTGGTGGAATGATTG
CATCATTCTTTGGTGGGTGGCTTGGGGACACACTTGGAAGAATCAAAGCCATGTTAGTAGCAAACATT
CTGTCATTAGTTGGAGCTCTCTTGATGGGGTTTTCAAAATTGGGACCATCTCATATACTTATAATTGC
TGGAAGAAGCATATCAGGACTATATTGTGGGCTAATTTCAGGCCTGGTTCCTATGTATATCGGTGAAA
TTGCTCCAACCGCTCTCAGGGGAGCACTTGGCACTTTTCATCAGCTGGCCATCGTCACGGGCATTCTT
ATTAGTCAGATTATTGGTCTTGAATTTATCTTGGGCAATTATGATCTGTGGCACATCCTGCTTGGCCT
GTCTGGTGTGCGAGCCATCCTTCAGTCTCTGCTACTCTTTTTCTGTCCAGAAAGCCCCAGATACCTTT
ACATCAAGTTAGATGAGGAAGTCAAAGCAAAACAAAGCTTGAAAAGACTCAGAGGATATGATGATGTC
ACCAAAGATATTAATGAAATGAGAAAAGAAAGAGAAGAAGCATCGAGTGAGCAGAAAGTCTCTATAAT
TCAGCTCTTCACCAATTCCAGCTACCGACAGCCTATTCTAGTGGCACTGATGCTGCATGTGGCTCAGC
AATTTTCCGGAATCAATGGCATTTTTTACTACTCAACCAGCATTTTTCAGACGGCTGGTATCAGCAAA
CCTGTTTATGCAACCATTGGAGTTGGCGCTGTAAACATCGTTTTCACTGCTGTCTCTGTATTCCTTGT
GGAGAAGGCAGGGCGACGTTCTCTCTTTCTAATTGGAATGAGTGGGATGTTTGTTTGTGCCATCTTCA
TGTCAGTGGGACTTGTGCTGCTGAATAAGTTCTCTTGGATGAGTTATGTGAGCATGATAGCCATCTTC
CTCTTTGTCAGCTTCTTTGAAATTGGGCCAGGCCCGATCCCCTGGTTCATGGTGGCTGAGTTTTTCAG
TCAAGGACCACGTCCTGCTGCTTTAGCAATAGCTGCATTCAGCAATTGGACCTGCAATTTCATTGTAG
CTCTGTGTTTCCAGTACATTGCGGACTTCTGTGGACCTTATGTGTTTTTCCTCTTTGCTGGAGTGCTC
CTGGCCTTTACCCTGTTTACATTTTTTAAAGTTCCAGAAACCAAAGGAAAGTCTTTTGAGGAAATTGC
TGCAGAATTCCAAAAGAAGAGTGGCTCAGCCCACAGGCCAAAAGCTGCTGTAGAAATGAAATTCCTAG
GAGCTACAGAGACTGTGCTCGAG

| | | | |
|---|---|---|---|
| NOV10c, 246837941 | SEQ ID NO: 38 | 529 aa | MW at 57988.4kD |
| Protein Sequence | | | |

GSTMTEDKVTGTLVFTVITAVLGSFQFGYDIGVINAPQQVIISHYRHVLGVPLDDRKAINNYVINSTD
ELPTISYSMNPKPTPWAEEETVAAAQLITMLWSLSVSSFAVGGMIASFFGGWLGDTLGRIKAMLVANI
LSLVGALLMGFSKLGPSHILIIAGRSISGLYCGLISGLVPMYIGEIAPTALRGALGTFHQLAIVTGIL
ISQIIGLEFILGNYDLWHILLGLSGVRAILQSLLLFFCPESPRYLYIKLDEEVKAKQSLKRLRGYDDV
TKDINEMRKEREEASSEQKVSIIQLFTNSSYRQPILVALMLHVAQQFSGINGIFYYSTSIFQTAGISK
PVYATIGVGAVNMVFTAVSVFLVEKAGRRSLFLIGMSGMFVCAIFMSVGLVLLNKFSWMSYVSMIAIF
LFVSFFEIGPGPIPWFMVAEFFSQGPRPAALAIAAFSNWTCNFIVALCFQYIADFCGPYVFFLFAGVL
LAFTLFTFFKVPETKGKSFEEIAAEFQKKSGSAHRPKAAVEMKFLGATETVLE

| | | |
|---|---|---|
| NOV10d, CG56151-02 | SEQ ID NO: 39 | 1664 bp |
| DNA Sequence | ORF Start: ATG at 39 | ORF Stop: end of sequence |

CACAAGACCTGGAATTGACAGGACTCCCAACTAGTACAATGACAGAAGATAAGGTCACTGGGACCCTG
GTTTTCACTGTCATCACTGCTGTGCTGGGTTCCTTCCAGTTTGGATATGACATTGGTGTGATCAATGC
ACCTCAACAGGTAATAATATCTCACTATAGACATGTTTTGGGTGTTCCACTGGATGACCGAAAAGCTA
TCAACAACTATGTTATCAACAGTACAGATGAACTGCCCACAATCTCATACTCAATGAACCCAAAACCA
ACCCCTTGGGCTGAGGAAGAGACTGTGGCAGCTGCTCAACTAATCACCATGCTCTGGTCCCTGTCTGT
ATCCAGCTTTGCAGTTGGTGGAATGACTGCATCATTCTTTGGTGGGTGGCTTGGGGACACACTTGGAA
GAATCAAAGCCATGTTAGTAGCAAACATTCTGTCATTAGTTGGAGCTCTCTTGATGGGGTTTTCAAAA
TTGGGACCATCTCATATACTTATAATTGCTGGAAGAAGCATATCAGGACTATATTGTGGGCTAATTTC
AGGCCTGGTTCCTATGTATATCGGTGAAATTGCTCCAACCGCTCTCAGGGGAGCACTTGGCACTTTTC
ATCAGCTGGCCATCGTCACGGGCATTCTTATTAGTCAGATTATTGGTCTTGAATTTATCTTGGGCAAT
TATGATCTGTGGCACATCCTGCTTGGCCTGTCTGGTGTGCGAGCCATCCTTCAGTCTCTGCTACTCTT
TTTCTGTCCAGAAAGCCCCAGATACCTTTACATCAAGTTAGATGAGGAAGTCAAAGCAAAACAAAGCT
TGAAAAGACTCAGAGGATATGATGATGTCACCAAAGATATTAATGAAATGAGAAAAGAAAGAGAAGAA
GCATCGAGTGAGCAGAAAGTCTCTATAATTCAGCTCTTCACCAATTCCAGCTACCGACAGCCTATTCT
AGTGGCACTGATGCTGCATGTGGCTCAGCAATTTTCCGGAATCAATGGCATTTTTTACTACTCAACCA
GCATTTTTCAGACGGCTGGTATCAGCAAACCTGTTTATGCAACCATTGGAGTTGGCGCTGTAAACATG
GTTTTCACTGCTGTCTCTGTATTCCTTGTGGAGAAGGCAGGGCGACGTTCTCTCTTTCTAATTGGAAT
GAGTGGGATGTTTGTTTGTGCCATCTTCATGTCAGTGGGACTTGTGCTGCTGAATAAGTTCTCTTGGA
TGAGTTATGTGAGCATGATAGCCATCTTCCTCTTTGTCAGCTTCTTTGAAATTGGGCCAGGCCCGATC
CCCTGGTTTATGGTGGCTGAGTTTTTCAGTCAAGGACCACGTCCTGCTGCTTTAGCAATAGCTGCATT
CAGCAATTGGACCTGCAATTTCATTGTAGCTCTGTGTTTCCAGTACATTGCGGACTTCTGTGGACCTT
ATGTGTTTTTCCTCTTTGCTGGAGTGCTCCTGGCCTTTACCCTGTTTACATTTTTTAAAGTTCCAGAA
ACCAAAGGAAAGTCTTTTGAGGAAATTGCTGCAGAATTCCAAAAGAAGAGTGGCTCAGCCCACAGGCC
AAAAGCTGCTGTAGAAATGAAATTCCTAGGAGCTACAGAGACTGTGTAAAAAAAAAACCCTGCTTTTT
GGCATGAACAGAAACAATAAGGGAACCGTCTG

| | | | |
|---|---|---|---|
| NOV10d, CG56151-02 | SEQ ID NO: 40 | 524 aa | MW at 57488.8kD |
| Protein Sequence | | | |

MTEDKVTGTLVFTVITAVLGSFQFGYDIGVINAPQQVIISHYRHVLGVPLDDRKAINNYVINSTDELP
TISYSMNPKPTPWAEEETVAAAQLITMLWSLSVSSFAVGGMTASFFGGWLGDTLGRIKAMLVANILSL
VGALLMGFSKLGPSHILIIAGRSISGLYCGLISGLVPMYIGEIAPTALRGALGTFHQLAIVTGILISQ
IIGLEFILGNYDLWHILLGLSGVRAILQSLLLFFCPESPRYLYIKLDEEVKAKQSLKRLRGYDDVTKD
INEMRKEREEASSEQKVSIIQLFTNSSYRQPILVALMLHVAQQFSGINGIFYYSTSIFQTAGISKPVY
ATIGVGAVNMVFTAVSVFLVEKAGRRSLFLIGMSGMFVCAIFMSVGLVLLNKFSWMSYVSMIAIFLFV
SFFEIGPGPIPWFMVAEFFSQGPRPAALAIAAFSNWTCNFIVALCFQYIADFCGPYVFFLFAGVLLAF
TLFTFFKVPETKGKSFEEIAAEFQKKSGSAHRPKAAVEMKFLGATETV

| | | |
|---|---|---|
| NOV10e, CG56151-03 | SEQ ID NO: 41 | 1587 bp |
| DNA Sequence | ORF Start: ATG at 10 | ORF Stop: end of sequence |

GGATCCACCATGACAGAAGATAAGGTCACTGGGACCCTGGTTTTCACTGTCATCACTGCTGTGCTGGG
TTCCTTCCAGTTTGGATATGACATTGGTGTGATCAATGCACCTCAACAGGTAATAATATCTCACTATA
GACATGTTTTGGGTGTTCCACTGGATGACCGAAAAGCTATCAACAACTATGTTATCAACAGTACAGAT
GAACTGCCCACAATCTCATACTCAATGAACCCAAAACCAACCCCTTGGGCTGAGGAAGAGACTGTGGC
AGCTGCTCAACTAATCACCATGCTCTGGTCCCTGTCTGTATCCAGCTTTGCAGTTGGTGGAATGACTG
CATCATTCTTTGGTGGGTGGCTTGGGGACACACTTGGAAGAATCAAAGCCATGTTAGTAGCAAACATT
CTGTCATTAGTTGGAGCTCTCTTGATGGGGTTTTCAAAATTGGGACCATCTCATATACTTATAATTGC
TGGAAGAAGCATATCAGGACTATATTGTGGGCTAATTTCAGGCCTGGTTCCTATGTATATCGGTGAAA
TTGCTCCAACCGCTCTCAGGGGAGCACTTGGCACTTTTCATCAGCTGGCCATCGTCACGGGCATTCTT
ATTAGTCAGATTATTGGTCTTGAATTTATCTTGGGCAATTATGATCTGTGGCACATCCTGCTTGGCCT
GTCTGGTGTGCGAGCCATCCTTCAGTCTCTGCTACTCTTTTTCTGTCCAGAAAGCCCCAGATACCTTT
ACATCAAGTTAGATGAGGAAGTCAAAGCAAAACAAAGCTTGAAAAGACTCAGAGGATATGATGATGTC
ACCAAAGATATTAATGAAATGAGAAAAGAAAGAGAAGAAGCATCGAGTGAGCAGAAAGTCTCTATAAT
TCAGCTCTTCACCAATTCCAGCTACCGACAGCCTATTCTAGTGGCACTGATGCTGCATGTGGCTCAGC
AATTTTCCGGAATCAATGGCATTTTTTACTACTCAACCAGCATTTTTCAGACGGCTGGTATCAGCAAA
CCTGTTTATGCAACCATTGGAGTTGGCGCTGTAAACATGGTTTTCACTGCTGTCTCTGTATTCCTTGT
GGAGAAGGCAGGGCGACGTTCTCTCTTTCTAATTGGAATGAGTGGGATGTTTGTTTGTGCCATCTTCA
TGTCAGTGGGACTTGTGCTGCTGAATAAGTTCTCTTGGATGAGTTATGTGAGCATGATAGCCATCTTC
CTCTTTGTCAGCTTCTTTGAAATTGGGCCAGGCCCGATCCCCTGGTTTATGGTGGCTGAGTTTTTCAG
TCAAGGACCACGTCCTGCTGCTTTAGCAATAGCTGCATTCAGCAATTGGACCTGCAATTTCATTGTAG
CTCTGTGTTTCCAGTACATTGCGGACTTCTGTGGACCTTATGTGTTTTTCCTCTTTGCTGGAGTGCTC
CTGGCCTTTACCCTGTTTACATTTTTTAAAGTTCCAGAAACCAAAGGAAAGTCTTTTGAGGAAATTGC
TGCAGAATTCCAAAAGAAGAGTGGCTCAGCCCACAGGCCAAAAGCTGCTGTAGAAATGAAATTCCTAG
GAGCTACAGAGACTGTGCTCGAG

| | | | |
|---|---|---|---|
| NOV10e, CG56151-03 | SEQ ID NO: 42 | 524 aa | MW at 57488.8kD |
| Protein Sequence | | | |

MTEDKVTGTLVFTVITAVLGSFQFGYDIGVINAPQQVIISHYRHVLGVPLDDRKAINNYVINSTDELP
TISYSMNPKPTPWAEEETVAAAQLITMLWSLSVSSFAVGGMTASFFGGWLGDTLGRIKAMLVANILSL
VGALLMGFSKLGPSHILIIAGRSISGLYCGLISGLVPMYIGEIAPTALRGALGTFHQLAIVTGILISQ
IIGLEFILGNYDLWHILLGLSGVRAILQSLLLFFCPESPRYLYIKLDEEVKAKQSLKRLRGYDDVTKD
INEMRKEREEASSEQKVSIIQLFTNSSYRQPILVALMLHVAQQFSGINGIFYYSTSIFQTAGISKPVY
ATIGVGAVNMVFTAVSVFLVEKAGRRSLFLIGMSGMFVCAIFMSVGLVLLNKFSWMSYVSMIAIFLFV
SFFEIGPGPIPWFMVAEFFSQGPRPAALAIAAFSNWTCNFIVALCFQYIADFCGPYVFFLFAGVLLAF
TLFTFFKVPETKGKSFEEIAAEFQKKSGSAHRPKAAVEMKFLGATETV

| | | |
|---|---|---|
| NOV10f, CG56151-04 | SEQ ID NO: 43 | 1473 bp |
| DNA Sequence | ORF Start: at 7 | ORF Stop: end of sequence |

GGATCCACTGTCATCACTGCTGTGCTGGGTTCCTTCCAGTTTGGATATGACATTGGTGTGATCAATGC
ACCTCAACAGGTAATAATATCTCACTATAGACATGTTTTGGGTGTTCCACTGGATGACCGAAAAGCTA
TCAACAACTATGTTATCAACAGTACAGATGAACTGCCCACAATCTCATACTCAATGAACCCAAAACCA
ACCCCTTGGGCTGAGGAAGAGACTGTGGCAGCTGCTCAACTAATCACCATGCTCTGGTCCCTGTCTGT
ATCCAGCTTTGCAGTTGGTGGAATGACTGCATCATTCTTTGGTGGGTGGCTTGGGGACACACTTGGAA
GAATCAAAGCCATGTTAGTAGCAAACATTCTGTCATTAGTTGGAGCTCTCTTGATGGGGTTTTCAAAA
TTGGGACCATCTCATATACTTATAATTGCTGGAAGAAGCATATCAGGACTATATTGTGGGCTAATTTC
AGGCCTGGTTCCTATGTATATCGGTGAAATTGCTCCAACCGCTCTCAGGGGAGCACTTGGCACTTTTC
ATCAGCTGGCCATCGTCACGGGCATTCTTATTAGTCAGATTATTGGTCTTGAATTTATCTTGGGCAAT
TATGATCTGTGGCACATCCTGCTTGGCCTGTCTGGTGTGCGAGCCATCCTTCAGTCTCTGCTACTCTT
TTTCTGTCCAGAAAGCCCCAGATACCTTTACATCAAGTTAGATGAGGAAGTCAAAGCAAAACAAAGCT
TGAAAAGACTCAGAGGATATGATGATGTCACCAAAGATATTAATGAAATGAGAAAAGAAAGAGAAGAA
GCATCGAGTGAGCAGAAAGTCTCTATAATTCAGCTCTTCACCAATTCCAGCTACCGACAGCCTATTCT
AGTGGCACTGATGCTGCATGTGGCTCAGCAATTTTCCGGAATCAATGGCATTTTTTACTACTCAACCA
GCATTTTTCAGACGGCTGGTATCAGCAAACCTGTTTATGCAACCATTGGAGTTGGCGCTGTAAACATG
GTTTTCACTGCTGTCTCTGTATTCCTTGTGGAGAAGGCAGGGCGACGTTCTCTCTTTCTAATTGGAAT
GAGTGGGATGTTTGTTTGTGCCATCTTCATGTCAGTGGGACTTGTGCTGCTGAATAAGTTCTCTTGGA
TGAGTTATGTGAGCATGATAGCCATCTTCCTCTTTGTCAGCTTCTTTGAAATTGGGCCAGGCCCGATC
CCCTGGTTTATGGTGGCTGAGTTTTTCAGTCAAGGACCACGTCCTGCTGCTTTAGCAATAGCTGCATT
CAGCAATTGGACCTGCAATTTCATTGTAGCTCTGTGTTTCCAGTACATTGCGGACTTCTGTGGACCTT
ATGTGTTTTTCCTCTTTGCTGGAGTGCTCCTGGCCTTTACCCTGTTTACATTTTTTAAAGTTCCAGAA
ACCAAAGGAAAGTCTTTTGAGGAAATTGCTGCAGAATTCCTCGAG

| | | | |
|---|---|---|---|
| NOV10f, CG56151-04 | SEQ ID NO: 44 | 487 aa | MW at 53512.2kD |
| Protein Sequence | | | |

TVITAVLGSFQFGYDIGVINAPQQVIISHYRHVLGVPLDDRKAINNYVINSTDELPTISYSMNPKPTP
WAEEETVAAAQLITMLWSLSVSSFAVGGMTASFFGGWLGDTLGRIKAMLVANILSLVGALLMGFSKLG
PSHILIIAGRSISGLYCGLISGLVPMYIGEIAPTALRGALGTFHQLAIVTGILISQIIGLEFILGNYD
LWHILLGLSGVRAILQSLLLFFCPESPRYLYIKLDEEVKAKQSLKRLRGYDDVTKDINEMRKEREEAS
SEQKVSIIQLFTNSSYRQPILVALMLHVAQQFSGINGIFYYSTSIFQTAGISKPVYATIGVGAVNMVF
TAVSVFLVEKAGRRSLFLIGMSGMFVCAIFMSVGLVLLNKFSWMSYVSMIAIFLFVSFFEIGPGPIPW
FMVAEFFSQGPRPAALAIAAFSNWTCNFIVALCFQYIADFCGPYVFFLFAGVLLAFTLFTFFKVPETK
GKSFEEIAAEF

A ClustalW comparison of the above protein sequences yields the following sequence alignment shown in Table 10B.

TABLE 10B
Comparison of the NOV10 protein sequences.

| | |
|---|---|
| NOV10a | ---MTEDKVTGTLVFTVITAVLGSFQFGYDIGVINAPQQVIISHYRHVLGVPLDDRKAIN |
| NOV10b | GSTMTEDKVTGTLVFTVITAVLGSFQFGYDIGVINAPQQVIISHYRHVLGVPLDDRKAIN |
| NOV10c | GSTMTEDKVTGTLVFTVITAVLGSFQFGYDIGVINAPQQVIISHYRHVLGVPLDDRKAIN |
| NOV10d | ---MTEDKVTGTLVFTVITAVLGSFQFGYDIGVINAPQQVIISHYRHVLGVPLDDRKAIN |
| NOV10e | ---MTEDKVTGTLVFTVITAVLGSFQFGYDIGVINAPQQVIISHYRHVLGVPLDDRKAIN |
| NOV10f | ---------------TVITAVLGSFQFGYDIGVINAPQQVIISHYRHVLGVPLDDRKAIN |
| NOV10a | NYVINSTDELPTISYSMNPKPTPWAEEETVAAAQLITMLWSLSVSSFAVGGMTASFFGGW |
| NOV10b | NYVINSTDELPTISYSMNPKPTPWAEEETVAAAQLITMLWSLSVSSFAVGGMIASFFGGW |
| NOV10c | NYVINSTDELPTISYSMNPKPTPWAEEETVAAAQLITMLWSLSVSSFAVGGMIASFFGGW |
| NOV10d | NYVINSTDELPTISYSMNPKPTPWAEEETVAAAQLITMLWSLSVSSFAVGGMTASFFGGW |
| NOV10e | NYVINSTDELPTISYSMNPKPTPWAEEETVAAAQLITMLWSLSVSSFAVGGMTASFFGGW |
| NOV10f | NYVINSTDELPTISYSMNPKPTPWAEEETVAAAQLITMLWSLSVSSFAVGGMTASFFGGW |
| NOV10a | LGDTLGRIKAMLVANILSLVGALLMGFSKLGPSHILIIAGRSISGLYCGLISGLVPMYIG |
| NOV10b | LGDTLGRIKAMLVANILSLVGALLMGFSKLGPSHILIIAGRSISGLYCGLISGLVPMYIG |
| NOV10c | LGDTLGRIKAMLVANILSLVGALLMGFSKLGPSHILIIAGRSISGLYCGLISGLVPMYIG |
| NOV10d | LGDTLGRIKAMLVANILSLVGALLMGFSKLGPSHILIIAGRSISGLYCGLISGLVPMYIG |
| NOV10e | LGDTLGRIKAMLVANILSLVGALLMGFSKLGPSHILIIAGRSISGLYCGLISGLVPMYIG |
| NOV10f | LGDTLGRIKAMLVANILSLVGALLMGFSKLGPSHILIIAGRSISGLYCGLISGLVPMYIG |
| NOV10a | EIAPTALRGALGTFHQLAIVTGILISQIIGLEFILGNYDLWHILLGLSGVRAILQSLLLF |
| NOV10b | EIAPTALRGALGTFHQLAIVTGILISQIIGLEFILGNYDLWHILLGLSGVRAILQSLLLF |
| NOV10c | EIAPTALRGALGTFHQLAIVTGILISQIIGLEFILGNYDLWHILLGLSGVRAILQSLLLF |
| NOV10d | EIAPTALRGALGTFHQLAIVTGILISQIIGLEFILGNYDLWHILLGLSGVRAILQSLLLF |
| NOV10e | EIAPTALRGALGTFHQLAIVTGILISQIIGLEFILGNYDLWHILLGLSGVRAILQSLLLF |
| NOV10f | EIAPTALRGALGTFHQLAIVTGILISQIIGLEFILGNYDLWHILLGLSGVRAILQSLLLF |
| NOV10a | FCPESPRYLYIKLDEEVKAKQSLKRLRGYDDVTKDINEMRKEREEASSEQKVSIIQLFTN |
| NOV10b | FCPESPRYLYIKLDEEVKAKQILKRLRGYDDVTKDINEMRKEREEASSEQKVSIIQLFTN |
| NOV10c | FCPESPRYLYIKLDEEVKAKQSLKRLRGYDDVTKDINEMRKEREEASSEQKVSIIQLFTN |
| NOV10d | FCPESPRYLYIKLDEEVKAKQSLKRLRGYDDVTKDINEMRKEREEASSEQKVSIIQLFTN |
| NOV10e | FCPESPRYLYIKLDEEVKAKQSLKRLRGYDDVTKDINEMRKEREEASSEQKVSIIQLFTN |
| NOV10f | FCPESPRYLYIKLDEEVKAKQSLKRLRGYDDVTKDINEMRKEREEASSEQKVSIIQLFTN |
| NOV10a | SSYRQPILVALMLHVAQQFSGINGIFYYSTSIFQTAGISKPVYATIGVGAVNMVFTAVSV |
| NOV10b | SSYRQPILVALMLHVAQQFSGINGIFYYSTSIFQTAGISKPVYATIGVGAVNMVFTAVSV |
| NOV10c | SSYRQPILVALMLHVAQQFSGINGIFYYSTSIFQTAGISKPVYATIGVGAVNMVFTAVSV |
| NOV10d | SSYRQPILVALMLHVAQQFSGINGIFYYSTSIFQTAGISKPVYATIGVGAVNMVFTAVSV |
| NOV10e | SSYRQPILVALMLHVAQQFSGINGIFYYSTSIFQTAGISKPVYATIGVGAVNMVFTAVSV |
| NOV10f | SSYRQPILVALMLHVAQQFSGINGIFYYSTSIFQTAGISKPVYATIGVGAVNMVFTAVSV |
| NOV10a | FLVEKAGRRSLFLIGMSGMFVCAIFMSVGLVLLNKFSWMSYVSMIAIFLFVSFFEIGPGP |
| NOV10b | FLVEKAGRRSLFLIGMSGMFVCAIFMSVGLVLLNKFSWMSYVSMIAIFLFVSFFEIGPGP |
| NOV10c | FLVEKAGRRSLFLIGMSGMFVCAIFMSVGLVLLNKFSWMSYVSMIAIFLFVSFFEIGPGP |
| NOV10d | FLVEKAGRRSLFLIGMSGMFVCAIFMSVGLVLLNKFSWMSYVSMIAIFLFVSFFEIGPGP |
| NOV10e | FLVEKAGRRSLFLIGMSGMFVCAIFMSVGLVLLNKFSWMSYVSMIAIFLFVSFFEIGPGP |
| NOV10f | FLVEKAGRRSLFLIGMSGMFVCAIFMSVGLVLLNKFSWMSYVSMIAIFLFVSFFEIGPGP |
| NOV10a | IPWFMVAEFFSQGPRPAALAIAAFSNWTCNFIVALCFQYIADFCGPYVFFLFAGVLLAFT |
| NOV10b | IPWFMVAEFFSQGPRPAALAIAAFSNWTCNFIVALCFQYIADFCGPYVFFLFAGVLLAFT |
| NOV10c | IPWFMVAEFFSQGPRPAALAIAAFSNWTCNFIVALCFQYIADFCGPYVFFLFAGVLLAFT |
| NOV10d | IPWFMVAEFFSQGPRPAALAIAAFSNWTCNFIVALCFQYIADFCGPYVFFLFAGVLLAFT |
| NOV10e | IPWFMVAEFFSQGPRPAALAIAAFSNWTCNFIVALCFQYIADFCGPYVFFLFAGVLLAFT |
| NOV10f | IPWFMVAEFFSQGPRPAALAIAAFSNWTCNFIVALCFQYIADFCGPYVFFLFAGVLLAFT |
| NOV10a | LFTFFKVPETKGKSFEEIAAEFQKKSGSAHRPKAAVEMKFLGATETV-- |
| NOV10b | LFTFFKVPETKGKSFEEIAAEFQKKSGSAHRPKAAVEMKFLGATETVLE |
| NOV10c | LFTFFKVPETKGKSFEEIAAEFQKKSGSAHRPKAAVEMKFLGATETVLE |
| NOV10d | LFTFFKVPETKGKSFEEIAAEFQKKSGSAHRPKAAVEMKFLGATETV-- |
| NOV10e | LFTFFKVPETKGKSFEEIAAEFQKKSGSAHRPKAAVEMKFLGATETV-- |
| NOV10f | LFTFFKVPETKGKSFEEIAAEF--------------------------- |
| NOV10a | (SEQ ID NO: 34) |
| NOV10b | (SEQ ID NO: 36) |
| NOV10c | (SEQ ID NO: 38) |
| NOV10d | (SEQ ID NO: 40) |
| NOV10e | (SEQ ID NO: 42) |
| NOV10f | (SEQ ID NO: 44) |

Further analysis of the NOV10a protein yielded the following properties shown in Table 10C.

TABLE 10C
Protein Sequence Properties NOV10a

| SignalP analysis: | Cleavage site between residues 21 and 22 | | | | |
|---|---|---|---|---|---|
| PSORT II analysis: | PSG: a new signal peptide prediction method | | | | |
| | N-region: length 5; pos. chg 1; neg. chg 2 | | | | |
| | H-region: length 21; peak value 0.00 | | | | |
| | PSG score: −4.40 | | | | |
| | GvH: von Heijne's method for signal seq. recognition | | | | |
| | GvH score (threshold: −2.1): −2.99 | | | | |
| | possible cleavage site: between 20 and 21 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 10 | | | | |
| | INTEGRAL | Likelihood = | −2.66 | Transmembrane | 6-22 |
| | INTEGRAL | Likelihood = | −5.36 | Transmembrane | 128-144 |
| | INTEGRAL | Likelihood = | −1.44 | Transmembrane | 152-168 |
| | INTEGRAL | Likelihood = | −4.88 | Transmembrane | 196-212 |
| | INTEGRAL | Likelihood = | −2.28 | Transmembrane | 220-236 |
| | INTEGRAL | Likelihood = | −4.09 | Transmembrane | 343-356 |
| | INTEGRAL | Likelihood = | −6.85 | Transmembrane | 374-390 |
| | INTEGRAL | Likelihood = | −3.66 | Transmembrane | 398-414 |
| | INTEGRAL | Likelihood = | 0.10 | Transmembrane | 436-452 |
| | INTEGRAL | Likelihood = | −8.12 | Transmembrane | 465-481 |
| | PERIPHERAL | Likelihood = | 0.90 (at 92) | | |
| | ALOM score: −8.12 (number of TMSs: 10) | | | | |
| | MTOP: Prediction of membrane topology (Hartmann et al.) | | | | |
| | Center position for calculation: 13 | | | | |
| | Charge difference: −0.5 C(−0.5)-N( 0.0) | | | | |
| | N >= C: N-terminal side will be inside | | | | |
| | >>> membrane topology: type 3a | | | | |
| | MITDISC: discrimination of mitochondrial targeting seq | | | | |
| | R content: | 0 | Hyd Moment (75): | 9.24 | |
| | Hyd Moment (95): | 7.63 | G content: | 0 | |
| | D/E content: | 2 | S/T content: | 1 | |

| Score: −6.13 | | |
|---|---|---|
| Gavel: prediction of cleavage sites for mitochondrial preseq | | |
| cleavage site motif not found | | |
| NUCDISC: discrimination of nuclear localization signals | | |
| pat4: none | | |
| pat7: none | | |
| bipartite: none | | |
| content of basic residues: 7.3% | | |
| NLS Score: −0.47 | | |
| KDEL: ER retention motif in the C-terminus: none | | |
| ER Membrane Retention Signals: none | | |
| SKL: peroxisomal targeting signal in the C-terminus: none | | |
| PTS2: 2nd peroxisomal targeting signal: none | | |
| VAC: possible vacuolar targeting motif: none | | |
| RNA-binding motif: none | | |
| Actinin-type actin-binding motif: | | |
| type 1: none | | |
| type 2: none | | |
| NMYR: N-myristoylation pattern: none | | |
| Prenylation motif: none | | |
| memYQRL: transport motif from cell surface to Golgi: none | | |
| Tyrosines in the tail: none | | |
| Dileucine motif in the tail: none | | |
| checking 63 PROSITE DNA binding motifs: none | | |
| checking 71 PROSITE ribosomal protein motifs: none | | |
| checking 33 PROSITE prokaryotic DNA binding motifs: none | | |
| NNCN: Reinhardt's method for Cytoplasmic/Nuclear discrimination | | |
| Prediction: cytoplasmic | | |
| Reliability: 94.1 | | |
| COIL: Lupas's algorithm to detect coiled-coil regions | | |
| 266 | D | 0.57 |
| 267 | D | 0.57 |
| 268 | V | 0.57 |
| 269 | T | 0.57 |
| 270 | K | 0.57 |
| 271 | D | 0.57 |
| 272 | I | 0.57 |
| 273 | N | 0.57 |
| 274 | E | 0.57 |
| 275 | M | 0.57 |
| 276 | R | 0.57 |
| 277 | K | 0.57 |
| 278 | E | 0.57 |
| 279 | R | 0.57 |
| 280 | E | 0.57 |
| 281 | E | 0.57 |
| 282 | A | 0.57 |
| 283 | S | 0.57 |
| 284 | S | 0.57 |
| 285 | E | 0.57 |
| 286 | Q | 0.57 |
| 287 | K | 0.57 |
| 288 | V | 0.57 |
| 289 | S | 0.57 |
| 290 | I | 0.57 |
| 291 | I | 0.57 |
| 292 | Q | 0.57 |
| 293 | L | 0.57 |

total: 28 residues
Final Results (k = 9/23):
66.7%: endoplasmic reticulum
22.2%: mitochondrial
11.1%: nuclear
>> prediction for CG56151-01 is end (k = 9)

A search of the NOV10a protein against the Geneseq database, a proprietary database that contains sequences published in patents and patent publication, yielded several homologous proteins shown in Table 10D.

TABLE 10D
Geneseq Results for NOV10a

| | | NOV10a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB05602 | Human glucose transporter | 1 . . . 524 | 524/524 (100%) | 0.0 |
| | protein SEQ ID NO: 12 - Homo | 1 . . . 524 | 524/524 (100%) | |
| | sapiens, 524 aa. [US6323002-B1, | | | |
| | 27 NOV. 2001] | | | |
| ABG76953 | Human protein, homologous to | 1 . . . 524 | 524/524 (100%) | 0.0 |
| | glucose transporter type 2, | 1 . . . 524 | 524/524 (100%) | |
| | designated NOV5 - Homo | | | |
| | sapiens, 524 aa. [WO200255705- | | | |
| | A2, 18 JUL. 2002] | | | |
| AAW17836 | Rat glucose transporter GLUT-2 - | 1 . . . 524 | 427/524 (81%) | 0.0 |
| | Rattus sp, 522 aa. | 1 . . . 522 | 469/524 (89%) | |
| | [WO9715668-A2, 01 MAY | | | |
| | 1997] | | | |
| AAY27288 | Glucose transporter protein | 3 . . . 502 | 333/507 (65%) | 0.0 |
| | GLUT2 - Homo sapiens, 534 aa. | 10 . . . 515 | 407/507 (79%) | |
| | [US5942398-A, 24 AUG. 1999] | | | |
| AAB30522 | Amino acid sequence of a | 6 . . . 514 | 288/509 (56%) | e−162 |
| | consensus GLUT polypeptide - | 10 . . . 483 | 372/509 (72%) | |
| | Synthetic, 493 aa. [US6136547- | | | |
| | A, 24 OCT. 2000] | | | |

In a BLAST search of public sequence databases, the NOV10a protein was found to have homology to the proteins shown in the BLASTP data in Table 10E.

TABLE 10E
Public BLASTP Results for NOV10a

| | | NOV10a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P11168 | Solute carrier family 2, facilitated | 1 . . . 524 | 524/524 (100%) | 0.0 |
| | glucose transporter, member 2 | 1 . . . 524 | 524/524 (100%) | |
| | (Glucose transporter type 2, liver) - | | | |
| | Homo sapiens (Human), 524 aa. | | | |
| P14246 | Solute carrier family 2, facilitated | 1 . . . 524 | 429/524 (81%) | 0.0 |
| | glucose transporter, member 2 | 1 . . . 523 | 471/524 (89%) | |
| | (Glucose transporter type 2, liver) - | | | |
| | Mus musculus (Mouse), 523 aa. | | | |
| P12336 | Solute carrier family 2, facilitated | 1 . . . 524 | 427/524 (81%) | 0.0 |
| | glucose transporter, member 2 | 1 . . . 522 | 469/524 (89%) | |
| | (Glucose transporter type 2, liver) - | | | |
| | Rattus norvegicus (Rat), 522 aa. | | | |
| S06920 | glucose transport protein, hepatic - | 1 . . . 524 | 426/524 (81%) | 0.0 |
| | mouse, 523 aa. | 1 . . . 523 | 468/524 (89%) | |
| Q90592 | Solute carrier family 2, facilitated | 3 . . . 502 | 333/506 (65%) | 0.0 |
| | glucose transporter, member 2 | 10 . . . 514 | 407/506 (79%) | |
| | (Glucose transporter type 2, liver) - | | | |
| | Gallus gallus (Chicken), 533 aa. | | | |

PFam analysis predicts that the NOV10a protein contains the domains shown in the Table 10F.

TABLE 10F
Domain Analysis of NOV10a

| | | Identities/ | |
|---|---|---|---|
| | NOV10a | Similarities | |
| Pfam | Match | for the | Expect |
| Domain | Region | Matched Region | Value |
| OATP_C | 10 . . . 394 | 70/474 (15%) | 0.2 |
| | | 251/474 (53%) | |
| sugar_tr | 13 . . . 499 | 208/521 (40%) | 1.4e−201 |
| | | 436/521 (84%) | |

### Example 11

The NOV11 clone was analyzed, and the nucleotide and encoded polypeptide sequences are shown in Table 11A.

TABLE 11A
NOV11 Sequence Analysis

| | | |
|---|---|---|
| NOV11a, CG56155-02 | SEQ ID NO: 45 | 2038 bp |
| DNA Sequence | ORF Start: ATG at 10 | ORF Stop: end of sequence |

GTTTTCAGAATGATTTTATTCAAGCAAGCAACTTATTTCATTTCCTTGTTTGCTACAGTTTCCTGTGG
ATGTCTGACTCAACTCTATGAAAACGCCTTCTTCAGAGGTGGGGATGTAGCTTCCATGTACACCCCAA
ATGCCCAATACTGCCAGATGAGGTGCACATTCCACCCAAGGTGTTTGCTATTCAGTTTTCTTCCAGCA
AGTTCAATCAATGACATGGAGAAAAGGTTTGGTTGCTTCTTGAAAGATAGTGTTACAGGAACCCTGCC
AAAAGTACATCGAACAGGTGCAGTTTCTGGACATTCCTTGAAGCAATGTGGTCATCAAATAAGTGCTT
GCCATCGAGACATTTATAAAGGAGTTGATATGAGAGGAGTCAATTTTAATGTGTCTAAGGTTAGCAGT
GTTGAAGAATGCCAAAAAAGGTGCACCAATAACATTCGCTGCCAGTTTTTTTCATATGCCACGCAAAC
ATTTCACAAGGCAGAGTACCGGAACAATTGCCTATTAAAGTACAGTCCCGGAGGAACACCTACCGCTA
TAAAGGTGCTGAGTAACGTGGAATCTGGATTCTCACTGAAGCCCTGTGCCCTTTCAGAAATTGGTTGC
CACATGAACATCTTCCAGCATCTTGCGTTCTCAGATGTGGATGTTGCCAGGTTTCTCACTCCAGATGC
TTTTGTGTGTCGGACCATCTGCACCTATCACCCCAACTGCCTCTTCTTTACATTCTATACAAATGTAT
GGAAAATCGAGTCACAAAGAAATGTTTGTCTTCTTAAAACATCTGAAAGTGGCACACCAAGTTCCTCT
ACTCCTCAAGAAAACACCATATCTGGATATAGCCTTTTAACCTGCAAAAGAACTTTACCTGAACCCTG
CCATTCTAAAATTTACCCGGGAGTTGACTTTGGAGGAGAAGAATTGAATGTGACTTTTGTTAAAGGAG
TGAATGTTTGCCAAGAGACTTGCACAAAGATGATTCGCTGTCAGTTTTTCACTTATTCTTTACTCCCA
GAAGACTGTAAGGAAGAGAAGTGTAAGTGTTTCTTAAGATTATCTATGGATGGTTCTCCAACTAGGAT
TGCGTATGGGACACAAGGGAGCTCTGGTTACTCTTTGAGATTGTGTAACACTGGGGACAACGCTGTCT
GCACAACAAAAACAAGCACACGCATTGTTGGAGGAACAAACTCTTCTTGGGGAGAGTGGCCCTGGCAG
GTGAGCCTGCAGGTGAAGCTGACAGCTCAGAGGCACCTGTGTGGAGGGTCACTCATAGGACACCAGTG
GGTCCTCACTGCTGCCCACTGCTTTGATGGGCTTCCCCTGCAGGATGTTTGGCGCATCTATAGTGGCA
TTTTAAATCTGTCAGACATTACAAAAGATACACCTTTCTCACAAATAAAAGAGATTATTATTCACCAA
AACTATAAAGTCTCAGAAGGGAATCATGATATCGCCTTGATAAAACTCCAGGCTCCTTTGAATTACAC
TGAATTCCAAAAACCAATATGCCTACCTTCCAAAGGTGACACAAGCACAATTTATACCAACTGTTGGG
TAACCGGATGGGGCTTCTCGAAGGAGAAAGGTGAAATCCAAAATATTCTACAAAAGGTAAATATTCCT
TTGGTAACAAATGAAGAATGCCAGAAAAGATATCAAGATTATAAAATAACCCAACGGATGGTCTGTGC
TGGCTATAAAGAAGGGGGAAAAGATGCTTGTAAGGGAGATTCAGGTGGTCCCTTAGTTTGCAAACACA
ACGGAATGTGGCGTTTGGTGGGCATCACCAGCTGGGGTGAAGGCTGTGCCCGCAGGGAGCAACCTGGT
GTCTACACCAAAGTCGCTGAGTACATGGACTGGATTTTAGAGAAAACACAGAGCAGTGATGGAAAAGC
TCAGATGCAGTCACCAGCATGAGAAGCAGTCCAGAGTCTAGGCAATTTTTACAACCTGAGTTCAAGTC
AAATTCTGAGCCTGGGGGGTCCTCATCTGCAAAGCATGAAGAGTGGCATCTTCTTTGCATCCTAAG

| | | | |
|---|---|---|---|
| NOV11a, CG56155-02 | SEQ ID NO: 46 | 638 aa | MW at 71401.1kD |
| Protein Sequence | | | |

MILFKQATYFISLFATVSCGCLTQLYENAFFRGGDVASMYTPNAQYCQMRCTFHPRCLLFSFLPASSI
NDMEKRFGCFLKDSVTGTLPKVHRTGAVSGHSLKQCGHQISACHRDIYKGVDMRGVNFNVSKVSSVEE
CQKRCTNNIRCQFFSYATQTFHKAEYRNNCLLKYSPGGTPTAIKVLSNVESGFSLKPCALSEIGCHMN
IFQHLAFSDVDVARFLTPDAFVCRTICTYHPNCLFFTFYTNVWKIESQRNVCLLKTSESGTPSSSTPQ
ENTISGYSLLTCKRTLPEPCHSKIYPGVDFGGEELNVTFVKGVNVCQETCTKMIRCQFFTYSLLPEDC
KEEKCKCFLRLSMDGSPTRIAYGTQGSSGYSLRLCNTGDNAVCTTKTSTRIVGGTNSSWGEWPWQVSL
QVKLTAQRHLCGGSLIGHQWVLTAAHCFDGLPLQDVWRIYSGILNLSDITKDTPFSQIKEIIIHQNYK
VSEGNHDIALIKLQAPLNYTEFQKPICLPSKGDTSTIYTNCWVTGWGFSKEKGEIQNILQKVNIPLVT
NEECQKRYQDYKITQRMVCAGYKEGGKDACKGDSGGPLVCKHNGMWRLVGITSWGEGCARREQPGVYT
KVAEYMDWILEKTQSSDGKAQMQSPA

| | | |
|---|---|---|
| NOV11b, 227803167 | SEQ ID NO: 47 | 1869 bp |
| DNA Sequence | ORF Start: at 1 | ORF Stop: end of sequence |

GGATCCGGATGTCTGACTCAACTCTATGAAAACGCCTTCTTCAGAGGTGGGGATGTAGCTTCCATGTA
CACCCCAAATGCCCAATACTGCCAGATGAGGTGCACATTCCACCCAAGGTGTTTGCTATTCAGTTTTC
TTCCAGCAAGTTCAATCAATGACATGGAGAAAAGGTTTGGTTGCTTCTTGAAAGATAGTGTTACAGGA
ACCCTGCCAAAAGTACATCGAACAGGTGCAGTTTCTGGACATTCCTTGAAGCAATGTGGTCATCAAAT
AAGTGCTTGCCATCGAGACATTTATAAAGGAGTTGATATGAGAGGAGTCAATTTTAATGTGTCTAAGG
TTAGCAGTGTTGAAGAATGCCAAAAAAGGTGCACCAGTAACATTCGCTGCCAGTTTTTTTCATATGCC
ACGCAAACATTTCACAAGGCAGAGTACCGGAACAATTGCCTATTAAAGTACAGTCCCGGAGGAACACC
TACCGCTATAAAGGTGCTGAGTAACGTGGAATCTGGATTCTCACTGAAGCCCTGTGCCCTTTCAGAAA
TTGGTTGCCACATGAACATCTTCCAGCATCTTGCGTTCTCAGATGTGGATGTTGCCAGGTTTCTCACT
CCAGATGCTTTTGTGTGTCGGACCATCTGCACCTATCACCCCAACTGCCTCTTCTTTACATTCTATAC
AAATGTATGGAAAATCGAGTCACAAAGAAATGTTTGTCTTCTTAAAACATCTGAAAGTGGCACACCAA
GTTCCTCTACTCCTCAAGAAAACACCATATCTGGATATAGCCTTTTAACCTGCAAAAGAACTTTACCT
GAACCCTGCCATTCTAAAATTTACCCGGGAGTTGACTTTGGAGGAGAAGAATTGAATGTGACTTTTGT
TAAAGGAGTGAATGTTTGCCAAGAGACTTGCACAAAGATGATTCGCTGTCAGTTTTTCACTTATTCTT
TACTCCCAGAAGACTGTAAGGAAGAGAAGTGTAAGTGTTTCTTAAGATTATCTATGGATGGTTCTCCA
ACTAGGATTGCGTATGGGACACAAGGGAGCTCTGGTTACTCTTTGAGATTGTGTAACACTGGGGACAA
CGCTGTCTGCACAACAAAAACAAGCACACGCATTGTTGGAGGAACAAACTCTTCTTGGGGAGAGTGGC
CCTGGCAGGTGAGCCTGCAGGTGAAGCTGACAGCTCAGAGGCATCTGTGTGGAGGGTCACTCATAGGA
CACCAGTGGGTCCTCACTGCTGCCCACTGCTTTGATGGGCTTCCCCTGCAGGATGTTTGGCGCATCTA
TAGTGGCATTTTAAATCTGTCAGACATTACAAAAGATACACCTTTCTCACAAATAAAAGAGATTATTA
TTCACCAAAACTATAAAGTCTCAGAAGGGAATCATGATATCGCCTTGATAAAACTCCAGGCTCCTTTG
AATTACACTGAATTCCAAAAACCAATATGCCTACCTTCCAAAGGTGACACAAGCACAATTTATACCAA
CTGTTGGGTAACCGGATGGGGCTTCTCGAAGGAGAAAGGTGAAATCCAAAATATTCTACAAAAGGTAA
ATATTCCTTTGGTAACAAATGAAGAATGCCAGAAAAGATATCAAGATTATAAAATAACCCAACGGATG
GTCTGTGCTGGCTATAAAGAAGGGGGAAAAGATGCTTGTAAGGGAGATTCAGGTGGTCCCTTAGTTTG
CAAACACAATGGAATGTGGCGTTTGGTGGGCATCACCAGCTGGGGTGAAGGCTGTGCCCGCAGGGAGC
AACCTGGTGTCTACACCAAAGTCGCTGAGTACATGGACTGGATTTTAGAGAAAACACAGAGCAGTGAT
GGAAAAGCTCAGATGCAGTCACCAGCACTCGAG

| | | | |
|---|---|---|---|
| NOV11b, 227803167 | SEQ ID NO: 48 | 623 aa | MW at 69594.8kD |
| Protein Sequence | | | |

GSGCLTQLYENAFFRGGDVASMYTPNAQYCQMRCTFHPRCLLFSFLPASSINDMEKRFGCFLKDSVTG
TLPKVHRTGAVSGHSLKQCGHQISACHRDIYKGVDMRGVNFNVSKVSSVEECQKRCTSNIRCQFFSYA
TQTFHKAEYRNNCLLKYSPGGTPTAIKVLSNVESGFSLKPCALSEIGCHMNIFQHLAFSDVDVARFLT
PDAFVCRTICTYHPNCLFFTFYTNVWKIESQRNVCLLKTSESGTPSSSTPQENTISGYSLLTCKRTLP
EPCHSKIYPGVDFGGEELNVTFVKGVNVCQETCTKMIRCQFFTYSLLPEDCKEEKCKCFLRLSMDGSP
TRIAYGTQGSSGYSLRLCNTGDNAVCTTKTSTRIVGGTNSSWGEWPWQVSLQVKLTAQRHLCGGSLIG
HQWVLTAAHCFDGLPLQDVWRIYSGILNLSDITKDTPFSQIKEIIIHQNYKVSEGNHDIALIKLQAPL
NYTEFQKPICLPSKGDTSTIYTNCWVTGWGFSKEKGEIQNILQKVNIPLVTNEECQKRYQDYKITQRM
VCAGYKEGGKDACKGDSGGPLVCKHNGMWRLVGITSWGEGCARREQPGVYTKVAEYMDWILEKTQSSD
GKAQMQSPALE

| | | |
|---|---|---|
| NOV11c, CG56155-01 | SEQ ID NO: 49 | 2245 bp |
| DNA Sequence | ORF Start: ATG at 72 | ORF Stop: end of sequence |

AGAACAGCTTGAAGACCGTTCATTTTTAAGTGACAAGAGACTCACCTCCAAGAAGCAATTGTGTTTTC
AGAATGATTTTATTCAAGCAAGCAACTTATTTCATTTCCTTGTTTGCTACAGTTTCCTGTGGATGTCT
GACTCAACTCTATGAAAACGCCTTCTTCAGAGGTGGGGATGTAGCTTCCATGTACACCCCAAATGCCC
AATACTGCCAGATGAGGTGCACATTCCACCCAAGGTGTTTGCTATTCAGTTTTCTTCCAGCAAGTTCA
ATCAATGACATGGAGAAAAGGTTTGGTTGCTTCTTGAAAGATAGTGTTACAGGAACCCTGCCAAAAGT
ACATCGAACAGGTGCAGTTTCTGGACATTCCTTGAAGCAATGTGGTCATCAAATAAGTGCTTGCCATC
GAGACATTTATAAAGGAGTTGATATGAGAGGAGTCAATTTTAATGTGTCTAAGGTTAGCAGTGTTGAA
GAATGCCAAAAAAGGTGCACCAATAACATTCGCTGCCAGTTTTTTTCATATGCCACGCAAACATTTCA
CAAGGCAGAGTACCGGAACAATTGCCTATTAAAGTACAGTCCCGGAGGAACACCTACCGCTATAAAGG
TGCTGAGTAACGTGGAATCTGGATTCTCACTGAAGCCCTGTGCCCTTTCAGAAATTGGTTGCCACATG
AACATCTTCCAGCATCTTGCGTTCTCAGATGTGGATGTTGCCAGGGTTCTCACTCCAGATGCTTTTGT
GTGTCGGACCATCTGCACCTATCACCCCAACTGCCTCTTCTTTACATTCTATACAAATGTATGGAAAA
TCGAGTCACAAAGAAATGTTTGTCTTCTTAAAACATCTGAAAGTGGCACACCAAGTTCCTCTACTCCT
CAAGAAAACACCATATCTGGATATAGCCTTTTAACCTGCAAAAGAACTTTACCTGAACCCTGCCATTC
TAAAATTTACCCGGGAGTTGACTTTGGAGGAGAAGAATTGAATGTGACTTTTGTTAAAGGAGTGAATG
TTTGCCAAGAGACTTGCACAAAGATGATTCGCTGTCAGTTTTTCACTTATTCTTTACTCCCAGAAGAC
TGTAAGGAAGAGAAGTGTAAGTGTTTCTTAAGATTATCTATGGATGGTTCTCCAACTAGGATTGCGTA
TGGGACACAAGGGAGCTCTGGTTACTCTTTGAGATTGTGTAACACTGGGGACAACTCTGTCTGCACAA
CAAAAACAAGCACACGCATTGTTGGAGGAACAAACTCTTCTTGGGGAGAGTGGCCCTGGCAGGTGAGC
CTGCAGGTGAAGCTGACAGCTCAGAGGCACCTGTGTGGAGGGTCACTCATAGGACACCAGTGGGTCCT
CACTGCTGCCCACTGCTTTGATGGGCTTCCCCTGCAGGATGTTTGGCGCATCTATAGTGGCATTTTAA
ATCTGTCAGACATTACAAAAGATACACCTTTCTCACAAATAAAAGAGATTATTATTCACCAAAACTAT
AAAGTCTCAGAAGGGAATCATGATATCGCCTTGATAAAACTCCAGGCTCCTTTGAATTACACTGAATT
CCAAAAACCAATATGCCTACCTTCCAAAGGTGACACAAGCACAATTTATACCAACTGTTGGGTAACCG
GATGGGGCTTCTCGAAGGAGAAAGGTGAAATCCAAAATATTCTACAAAAGGTAAATATTCCTTTGGTA
ACAAATGAAGAATGCCAGAAAAGATATCAAGATTATAAAATAACCCAACGGATGGTCTGTGCTGGCTA
TAAAGAAGGGGGAAAAGATGCTTGTAAGGGAGATTCAGGTGGTCCCTTAGTTTGCAAACACAACGGAA
TGTGGCGTTTGGTGGGCATCACAAGCTGGGGTGAAGGCTGTGCCCGCAGGGAGCAACCTGGTGTCTAC
ACCAAAGTCGCTGAGTACATGGACTGGATTTTAGAGAAAACACAGAGCAGTGATGGAAAAGCTCAGAT
GCAGTCACCAGCATGAGAAGCAGTCCAGAGTCTAGGCAATTTTTACAACCTGAGTTCAAGTCAAATTC
TGAGCCTGGGGGGTCCTCATCTGCAAAGCATGGAGAGTGGCATCTTCTTTGCATCCTAAGGACGAAAG
ACACAGTGCACTCAGAGCTGCTGAGGACAATGTCTGCTGAAGCCCGCTTTCAGCACGCCGTAACCAGG
GGCTGACAATGCGAGGTCGCAACTGAGATCTCCATGACTGTGTGTTGTGAAATAAAATGGTGAAAGAT
C

| | | | |
|---|---|---|---|
| NOV11c, CG56155-01 | SEQ ID NO: 50 | 638 aa | MW at 71369.0kD |
| Protein Sequence | | | |

MILFKQATYFISLFATVSCGCLTQLYENAFFRGGDVASMYTPNAQYCQMRCTFHPRCLLFSFLPASSI
NDMEKRFGCFLKDSVTGTLPKVHRTGAVSGHSLKQCGHQISACHRDIYKGVDMRGVNFNVSKVSSVEE
CQKRCTNNIRCQFFSYATQTFHKAEYRNNCLLKYSPGGTPTAIKVLSNVESGFSLKPCALSEIGCHMN
IFQHLAFSDVDVARVLTPDAFVCRTICTYHPNCLFFTFYTNVWKIESQRNVCLLKTSESGTPSSSTPQ
ENTISGYSLLTCKRTLPEPCHSKIYPGVDFGGEELNVTFVKGVNVCQETCTKMIRCQFFTYSLLPEDC
KEEKCKCFLRLSMDGSPTRIAYGTQGSSGYSLRLCNTGDNSVCTTKTSTRIVGGTNSSWGEWPWQVSL
QVKLTAQRHLCGGSLIGHQWVLTAAHCFDGLPLQDVWRIYSGILNLSDITKDTPFSQIKEIIIHQNYK
VSEGNHDIALIKLQAPLNYTEFQKPICLPSKGDTSTIYTNCWVTGWGFSKEKGEIQNILQKVNIPLVT
NEECQKRYQDYKITQRMVCAGYKEGGKDACKGDSGGPLVCKHNGMWRLVGITSWGEGCARREQPGVYT
KVAEYMDWILEKTQSSDGKAQMQSPA

| | | |
|---|---|---|
| NOV11d, CG56155-03 | SEQ ID NO: 51 | 1869 bp |
| DNA Sequence | ORF Start: at 7 | ORF Stop: end of sequence |

GGATCCGGATGTCTGACTCAACTCTATGAAAACGCCTTCTTCAGAGGTGGGGATGTAGCTTCCATGTA
CACCCCAAATGCCCAATACTGCCAGATGAGGTGCACATTCCACCCAAGGTGTTTGCTATTCAGTTTTC
TTCCAGCAAGTTCAATCAATGACATGGAGAAAAGGTTTGGTTGCTTCTTGAAAGATAGTGTTACAGGA
ACCCTGCCAAAAGTACATCGAACAGGTGCAGTTTCTGGACATTCCTTGAAGCAATGTGGTCATCAAAT
AAGTGCTTGCCATCGAGACATTTATAAAGGAGTTGATATGAGAGGAGTCAATTTTAATGTGTCTAAGG
TTAGCAGTGTTGAAGAATGCCAAAAAAGGTGCACCAGTAACATTCGCTGCCAGTTTTTTTCATATGCC
ACGCAAACATTTCACAAGGCAGAGTACCGGAACAATTGCCTATTAAAGTACAGTCCCGGAGGAACACC
TACCGCTATAAAGGTGCTGAGTAACGTGGAATCTGGATTCTCACTGAAGCCCTGTGCCCTTTCAGAAA
TTGGTTGCCACATGAACATCTTCCAGCATCTTGCGTTCTCAGATGTGGATGTTGCCAGGTTTCTCACT
CCAGATGCTTTTGTGTGTCGGACCATCTGCACCTATCACCCCAACTGCCTCTTCTTTACATTCTATAC
AAATGTATGGAAAATCGAGTCACAAAGAAATGTTTGTCTTCTTAAAACATCTGAAAGTGGCACACCAA
GTTCCTCTACTCCTCAAGAAAACACCATATCTGGATATAGCCTTTTAACCTGCAAAAGAACTTTACCT
GAACCCTGCCATTCTAAAATTTACCCGGGAGTTGACTTTGGAGGAGAAGAATTGAATGTGACTTTTGT
TAAAGGAGTGAATGTTTGCCAAGAGACTTGCACAAAGATGATTCGCTGTCAGTTTTTCACTTATTCTT
TACTCCCAGAAGACTGTAAGGAAGAGAAGTGTAAGTGTTTCTTAAGATTATCTATGGATGGTTCTCCA
ACTAGGATTGCGTATGGGACACAAGGGAGCTCTGGTTACTCTTTGAGATTGTGTAACACTGGGGACAA
CGCTGTCTGCACAACAAAAACAAGCACACGCATTGTTGGAGGAACAAACTCTTCTTGGGGAGAGTGGC
CCTGGCAGGTGAGCCTGCAGGTGAAGCTGACAGCTCAGAGGCACCTGTGTGGAGGGTCACTCATAGGA
CACCAGTGGGTCCTCACTGCTGCCCACTGCTTTGATGGGCTTCCCCTGCAGGATGTTTGGCGCATCTA
TAGTGGCATTTTAAATCTGTCAGACATTACAAAAGATACACCTTTCTCACAAATAAAAGAGATTATTA
TTCACCAAAACTATAAAGTCTCAGAAGGGAATCATGATATCGCCTTGATAAAACTCCAGGCTCCTTTG
AATTACACTGAATTCCAAAAACCAATATGCCTACCTTCCAAAGGTGACACAAGCACAATTTATACCAA
CTGTTGGGTAACCGGATGGGGCTTCTCGAAGGAGAAAGGTGAAATCCAAAATATTCTACAAAAGGTAA
ATATTCCTTTGGTAACAAATGAAGAATGCCAGAAAAGATATCAAGATTATAAAATAACCCAACGGATG
GTCTGTGCTGGCTATAAAGAAGGGGGAAAAGATGCTTGTAAGGGAGATTCAGGTGGTCCCTTAGTTTG
CAAACACAATGGAATGTGGCGTTTGGTGGGCATCACCAGCTGGGGTGAAGGCTGTGCCCGCAGGGAGC
AACCTGGTGTCTACACCAAAGTCGCTGAGTACATGGACTGGATTTTAGAGAAAACACAGAGCAGTGAT
GGAAAAGCTCAGATGCAGTCACCAGCACTCGAG

| | | | |
|---|---|---|---|
| NOV11d, CG56155-03 | SEQ ID NO: 52 | 619 aa | MW at 69208.4kD |
| Protein Sequence | | | |

GCLTQLYENAFFRGGDVASMYTPNAQYCQMRCTFHPRCLLFSFLPASSINDMEKRFGCFLKDSVTGTL
PKVHRTGAVSGHSLKQCGHQISACHRDIYKGVDMRGVNFNVSKVSSVEECQKRCTSNIRCQFFSYATQ
TFHKAEYRNNCLLKYSPGGTPTAIKVLSNVESGFSLKPCALSEIGCHMNIFQHLAFSDVDVARFLTPD
AFVCRTICTYHPNCLFFTFYTNVWKIESQRNVCLLKTSESGTPSSSTPQENTISGYSLLTCKRTLPEP
CHSKIYPGVDFGGEELNVTFVKGVNVCQETCTKMIRCQFFTYSLLPEDCKEEKCKCFLRLSMDGSPTR
IAYGTQGSSGYSLRLCNTGDNAVCTTKTSTRIVGGTNSSWGEWPWQVSLQVKLTAQRHLCGGSLIGHQ
WVLTAAHCFDGLPLQDVWRIYSGILNLSDITKDTPFSQIKEIIIHQNYKVSEGNHDIALIKLQAPLNY
TEFQKPICLPSKGDTSTIYTNCWVTGWGFSKEKGEIQNILQKVNIPLVTNEECQKRYQDYKITQRMVC
AGYKEGGKDACKGDSGGPLVCKHNGMWRLVGITSWGEGCARREQPGVYTKVAEYMDWILEKTQSSDGK
AQMQSPA

A ClustalW comparison of the above protein sequences yields the following sequence alignment shown in Table 11B.

TABLE 11B
Comparison of the NOV11 protein sequences.

| | |
|---|---|
| NOV11a | MILFKQATYFISLFATVSCGCLTQLYENAFFRGGDVASMYTPNAQYCQMRCTFHPRCLLF |
| NOV11b | -----------------GSGCLTQLYENAFFRGGDVASMYTPNAQYCQMRCTFHPRCLLF |
| NOV11c | MILFKQATYFISLFATVSCGCLTQLYENAFFRGGDVASMYTPNAQYCQMRCTFHPRCLLF |
| Nov11d | -------------------GCLTQLYENAFFRGGDVASMYTPNAQYCQMRCTFHPRCLLF |
| NOV11a | SFLPASSINDMEKRFGCFLKDSVTGTLPKVHRTGAVSGHSLKQCGHQISACHRDIYKGVD |
| NOV11b | SFLPASSINDMEKRFGCFLKDSVTGTLPKVHRTGAVSGHSLKQCGHQISACHRDIYKGVD |
| NOV11c | SFLPASSINDMEKRFGCFLKDSVTGTLPKVHRTGAVSGHSLKQCGHQISACHRDIYKGVD |
| NOV11d | SFLPASSINDMEKRFGCFLKDSVTGTLPKVHRTGAVSGHSLKQCGHQISACHRDIYKGVD |
| NOV11a | MRGVNFNVSKVSSVEECQKRCTNNIRCQFFSYATQTFHKAEYRNNCLLKYSPGGTPTAIK |
| NOV11b | MRGVNFNVSKVSSVEECQKRCTSNIRCQFFSYATQTFHKAEYRNNCLLKYSPGGTPTAIK |
| NOV11c | MRGVNFNVSKVSSVEECQKRCTNNIRCQFFSYATQTFHKAEYRNNCLLKYSPGGTPTAIK |
| NOV11d | MRGVNFNVSKVSSVEECQKRCTSNIRCQFFSYATQTFHKAEYRNNCLLKYSPGGTPTAIK |
| NOV11a | VLSNVESGFSLKPCALSEIGCHMNIFQHLAFSDVDVARFLTPDAFVCRTICTYHPNCLFF |
| NOV11b | VLSNVESGFSLKPCALSEIGCHMNIFQHLAFSDVDVARFLTPDAFVCRTICTYHPNCLFF |
| NOV11c | VLSNVESGFSLKPCALSEIGCHMNIFQHLAFSDVDVARVLTPDAFVCRTICTYHPNCLFF |
| NOV11d | VLSNVESGFSLKPCALSEIGCHMNIFQHLAFSDVDVARFLTPDAFVCRTICTYHPNCLFF |
| NOV11a | TFYTNVWKIESQRNVCLLKTSESGTPSSSTPQENTISGYSLLTCKRTLPEPCHSKIYPGV |
| NOV11b | TFYTNVWKIESQRNVCLLKTSESGTPSSSTPQENTISGYSLLTCKRTLPEPCHSKIYPGV |
| NOV11c | TFYTNVWKIESQRNVCLLKTSESGTPSSSTPQENTISGYSLLTCKRTLPEPCHSKIYPGV |
| NOV11d | TFYTNVWKIESQRNVCLLKTSESGTPSSSTPQENTISGYSLLTCKRTLPEPCHSKIYPGV |
| NOV11a | DFGGEELNVTFVKGVNVCQETCTKMIRCQFFTYSLLPEDCKEEKCKCFLRLSMDGSPTRI |
| NOV11b | DFGGEELNVTFVKGVNVCQETCTKMIRCQFFTYSLLPEDCKEEKCKCFLRLSMDGSPTRI |
| NOV11c | DFGGEELNVTFVKGVNVCQETCTKMIRCQFFTYSLLPEDCKEEKCKCFLRLSMDGSPTRI |
| NOV11d | DFGGEELNVTFVKGVNVCQETCTKMIRCQFFTYSLLPEDCKEEKCKCFLRLSMDGSPTRI |
| NOV11a | AYGTQGSSGYSLRLCNTGDNAVCTTKTSTRIVGGTNSSWGEWPWQVSLQVKLTAQRHLCG |
| NOV11b | AYGTQGSSGYSLRLCNTGDNAVCTTKTSTRIVGGTNSSWGEWPWQVSLQVKLTAQRHLCG |
| NOV11c | AYGTQGSSGYSLRLCNTGDNSVCTTKTSTRIVGGTNSSWGEWPWQVSLQVKLTAQRHLCG |
| NOV11d | AYGTQGSSGYSLRLCNTGDNAVCTTKTSTRIVGGTNSSWGEWPWQVSLQVKLTAQRHLCG |
| NOV11a | GSLIGHQWVLTAAHCFDGLPLQDVWRIYSGILNLSDITKDTPFSQIKEIIIHQNYKVSEG |
| NOV11b | GSLIGHQWVLTAAHCFDGLPLQDVWRIYSGILNLSDITKDTPFSQIKEIIIHQNYKVSEG |
| NOV11c | GSLIGHQWVLTAAHCFDGLPLQDVWRIYSGILNLSDITKDTPFSQIKEIIIHQNYKVSEG |
| Nov11d | GSLIGHQWVLTAAHCFDGLPLQDVWRIYSGILNLSDITKDTPFSQIKEIIIHQNYKVSEG |
| NOV11a | NHDIALIKLQAPLNYTEFQKPICLPSKGDTSTIYTNCWVTGWGFSKEKGEIQNILQKVNI |
| NOV11b | NHDIALIKLQAPLNYTEFQKPICLPSKGDTSTIYTNCWVTGWGFSKEKGEIQNILQKVNI |
| NOV11c | NHDIALIKLQAPLNYTEFQKPICLPSKGDTSTIYTNCWVTGWGFSKEKGEIQNILQKVNI |
| NOV11d | NHDIALIKLQAPLNYTEFQKPICLPSKGDTSTIYTNCWVTGWGFSKEKGEIQNILQKVNI |
| NOV11a | PLVTNEECQKRYQDYKITQRMVCAGYKEGGKDACKGDSGGPLVCKHNGMWRLVGITSWGE |
| NOV11b | PLVTNEECQKRYQDYKITQRMVCAGYKEGGKDACKGDSGGPLVCKHNGMWRLVGITSWGE |
| NOV11c | PLVTNEECQKRYQDYKITQRMVCAGYKEGGKDACKGDSGGPLVCKHNGMWRLVGITSWGE |
| NOV11d | PLVTNEECQKRYQDYKITQRMVCAGYKEGGKDACKGDSGGPLVCKHNGMWRLVGITSWGE |
| NOV11a | GCARREQPGVYTKVAEYMDWILEKTQSSDGKAQMQSPA-- |
| NOV11b | GCARREQPGVYTKVAEYMDWILEKTQSSDGKAQMQSPALE |
| NOV11c | GCARREQPGVYTKVAEYMDWILEKTQSSDGKAQMQSPA-- |
| NOV11d | GCARREQPGVYTKVAEYMDWILEKTQSSDGKAQMQSPA-- |
| NOV11a | (SEQ ID NO: 46) |
| NOV11b | (SEQ ID NO: 48) |
| NOV11c | (SEQ ID NO: 50) |
| NOV11d | (SEQ ID NO: 52) |

Further analysis of the NOV11a protein yielded the following properties shown in Table 11C.

TABLE 11C
Protein Sequence Properties NOV11a

| SignalP analysis: | Cleavage site between residues 20 and 21 | | | |
|---|---|---|---|---|
| PSORT II analysis: | PSG: a new signal peptide prediction method | | | |
| | N-region: length 5; pos. chg 1; neg. chg 0 | | | |
| | H-region: length 21; peak value 10.10 | | | |
| | PSG score: 5.70 | | | |
| | GvH: von Heijne's method for signal seq. recognition | | | |
| | GvH score (threshold: −2.1): −2.14 | | | |
| | possible cleavage site: between 23 and 24 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL Likelihood = 0.95 (at 7) | | | |
| | ALOM score: 0.95 (number of TMSs: 0) | | | |
| | MTOP: Prediction of membrane topology (Hartmann et al.) | | | |
| | Center position for calculation: 6 | | | |
| | Charge difference: −3.0 C(−1.0)-N(2.0) | | | |
| | N >= C: N-terminal side will be inside | | | |
| | MITDISC: discrimination of mitochondrial targeting seq | | | |
| | R content: | 0 | Hyd Moment (75): | 7.62 |
| | Hyd Moment (95): | 5.84 | G content: | 1 |
| | D/E content: | 1 | S/T content: | 5 |

```
Score: −3.79
Gavel: prediction of cleavage sites for mitochondrial preseq
cleavage site motif not found
NUCDISC: discrimination of nuclear localization signals
pat4: none
pat7: none
bipartite: none
content of basic residues: 10.5%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: found
TLPK at 86
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues
Final Results (k = 9/23):
26.1%: cytoplasmic
21.7%: mitochondrial
17.4%: nuclear
  8.7%: extracellular, including cell wall
  8.7%: vacuolar
  8.7%: Golgi
  8.7%: endoplasmic reticulum
>> prediction for CG56155-02 is cyt (k = 23)
```

A search of the NOV11a protein against the Geneseq database, a proprietary database that contains sequences published in patents and patent publication, yielded several homologous proteins shown in Table 11D.

TABLE 11D
Geneseq Results for NOV11a

| | | | Identities/ | |
|---|---|---|---|---|
| | | NOV11a | Similarities | |
| | | Residues/ | for the | |
| Geneseq | Protein/Organism/Length | Match | Matched | Expect |
| Identifier | [Patent #, Date] | Residues | Region | Value |
| ABG76951 | Human protein, homologous to | 1 . . . 638 | 638/638 (100%) | 0.0 |
| | Kallikrein, designated NOV3 - | 1 . . . 638 | 638/638 (100%) | |
| | Homo sapiens, 638 aa. | | | |
| | [WO200255705-A2, 18 JUL. | | | |
| | 2002] | | | |
| AAU68928 | Human protease domian of | 427 . . . 584 | 158/158 (100%) | 1e−92 |
| | kallikrein I - Homo sapiens, 158 | 1 . . . 158 | 158/158 (100%) | |
| | aa. [US6294663-B1, 25 SEP. | | | |
| | 2001] | | | |
| AAU82755 | Amino acid sequence of novel | 319 . . . 621 | 115/306 (37%) | 1e−56 |
| | human protease #54 - Homo | 513 . . . 797 | 172/306 (55%) | |
| | sapiens, 802 aa. [WO200200860- | | | |
| | A2, 03 JAN. 2002] | | | |
| AAB24052 | Human PRO618 protein | 319 . . . 621 | 115/306 (37%) | 1e−56 |
| | sequence SEQ ID NO: 24 - Homo | 513 . . . 797 | 172/306 (55%) | |
| | sapiens, 802 aa. [WO200053754- | | | |
| | A1, 14 SEP. 2000] | | | |
| AAB44266 | Human PRO618 (UNQ354) | 319 . . . 621 | 115/306 (37%) | 1e−56 |
| | protein sequence SEQ ID | 513 . . . 797 | 172/306 (55%) | |
| | NO: 169 - Homo sapiens, 802 aa. | | | |
| | [WO200053756-A2, 14 SEP. | | | |
| | 2000] | | | |

In a BLAST search of public sequence databases, the NOV11a protein was found to have homology to the proteins shown in the BLASTP data in Table 11E.

TABLE 11E
Public BLASTP Results for NOV11a

| | | | Identities/ | |
|---|---|---|---|---|
| | | | Similarities | |
| Protein | | | for the | |
| Accession | | NOV11a Residues/ | Matched | Expect |
| Number | Protein/Organism/Length | Match Residues | Portion | Value |
| P03952 | Plasma kallikrein precursor (EC | 1 . . . 638 | 636/638 (99%) | 0.0 |
| | 3.4.21.34) (Plasma prekallikrein) | 1 . . . 638 | 637/638 (99%) | |
| | (Kininogenin) (Fletcher factor) - | | | |
| | Homo sapiens (Human), 638 aa. | | | |
| O97506 | Kallikrein - Sus scrofa (Pig), 643 | 1 . . . 635 | 503/635 (79%) | 0.0 |
| | aa. | 9 . . . 643 | 568/635 (89%) | |
| Q8R0P5 | Kallikrein B, plasma 1 - Mus | 1 . . . 638 | 486/638 (76%) | 0.0 |
| | musculus (Mouse), 638 aa. | 1 . . . 638 | 554/638 (86%) | |
| P26262 | Plasma kallikrein precursor (EC | 1 . . . 638 | 485/638 (76%) | 0.0 |
| | 3.4.21.34) (Plasma prekallikrein) | 1 . . . 638 | 553/638 (86%) | |
| | (Kininogenin) (Fletcher factor) - | | | |
| | Mus musculus (Mouse), 638 aa. | | | |
| P14272 | Plasma kallikrein precursor (EC | 1 . . . 638 | 476/638 (74%) | 0.0 |
| | 3.4.21.34) (Plasma prekallikrein) | 1 . . . 638 | 549/638 (85%) | |
| | (Kininogenin) (Fletcher factor) - | | | |
| | Rattus norvegicus (Rat), 638 aa. | | | |

PFam analysis predicts that the NOV11a protein contains the domains shown in the Table 11F.

TABLE 11F
Domain Analysis of NOV11a

| | | Identities/ | |
|---|---|---|---|
| | NOV11a | Similarities | |
| Pfam | Match | for the | Expect |
| Domain | Region | Matched Region | Value |
| PAN | 21 . . . 104 | 19/112 (17%) | 7.9e−14 |
| | | 64/112 (57%) | |
| PAN | 111 . . . 194 | 23/111 (21%) | 4.8e−15 |
| | | 67/111 (60%) | |
| PAN | 201 . . . 284 | 19/111 (17%) | 4e−10 |
| | | 62/111 (56%) | |
| PAN | 292 . . . 375 | 22/111 (20%) | 2.3e−09 |
| | | 65/111 (59%) | |
| trypsin | 391 . . . 621 | 113/263 (43%) | 5.2e−92 |
| | | 196/263 (75%) | |

### Example 12

The NOV12 clone was analyzed, and the nucleotide and encoded polypeptide sequences are shown in Table 12A.

TABLE 12A
NOV12 Sequence Analysis

| | | |
|---|---|---|
| NOV12a, CG56262-01 | SEQ ID NO: 53 | 1551 bp |
| DNA Sequence | ORF Start: ATG at 108 | ORF Stop: end of sequence |

GCGGCCGCGGGAGCTGACCCTGCGGGGTCCCGGGGGGGGAGGGGGAGCCGCGAAGCCCCCACTGAGGC
CGCCGCTGCCGGGCCTCCCCTCCCCCCCGGGCGGGCGCCATGCGGGGGAGCCCGGGCGACGCGGAGCG
GCGGCAGCGCTGGGGTCGCCTGTTCGAGGAGCTGGACAGTAACAAGGATGGCCGCGTGGACGTGCACG
AGTTGCGCCAGGGGCTGGCCAGGCTGGGCGGGGGCAACCCAGACCCCGGCGCCCAACAGGGTATCTCC
TCTGAGGGTGATGCTGACCCAGATGGCGGGCTCGACCTGGAGGAATTTTCCCGCTATCTGCAGGAGCG
GGAACAGCGTCTGCTGCTCATGTTTCACAGTCTTGACCGGAACCAGGATGGTCACATTGATGTCTCTG
AGATCCAACAGAGTTTCCGAGCTCTGGGCATTTCCATCTCGCTGGAGCAGGCTGAGAAAATTTTGCAC
AGCATGGACCGAGACGGCACAATGACCATTGACTGGCAAGAATGGCGCGACCACTTCCTGTTGCATTC
GCTGGAAAATGTGGAGGACGTGCTGTATTTCTGGAAGCATTCCACGGTCCTGGACATTGGCGAGTGCC
TGACAGTGCCGGACGAGTTCTCAAAGCAAGAGAAGCTGACGGGCATGTGGTGGAAACAGCTGGTGGCC
GGCGCAGTGGCAGGTGCCGTGTCACGGACAGGCACGGCCCCTCTGGACCGCCTCAAGGTCTTCATTCA
GGTCCATGCCTCAAAGACCAACCGGCTGAACATCCTTGGGGGGCTTCGAAGCATGGTCCTTGAGGGAG
GCATCCGCTGCCTGTGGCGCGGCAATGGTATTAATGTACTCAAGATTGCCCCCGAGTCAGCTATCAAG
TTCATGGCCTATGAACAGGTGAGGAGGGCCATCCTGGGGCAGCAGGAGACACTGCATGTGCAGGAGCG
CTTCGTGGCTGGCTCCCTGGCTGGTGCCACAGCCCAAACCATCATTTACCCTATGGAGGTGCTGAAGA
CGCGGCTGACCTTGCGCCGGACGGGCCAGTATAAGGGGCTGCTGGACTGCGCCAGGCGTATCCTGGAG
AGGGAGGGGCCCCGTGCCTTCTACCGCGGCTACCTCCCCAACGTGCTGGGCATCATCCCCTATGCGGG
CATCGACCTGGCCGTCTACGAGGTCCTGAAGAACTGGTGGCTTCAGCAGTACAGCCACGACTCGGCAG
ACCCAGGCATCCTCGTGCTCCTGGCCTGCGGTACCATATCCAGCACCTGCGGCCAGATAGCCAGTTAC
CCGCTGGCCCTGGTCCGGACCCGCATGCAGGCACAAGCCTCCATCGAGGGTGGCCCCCAGCTGTCCAT
GCTGGGTCTGCTACGTCACATCCTGTCCCAGGAGGGCATGCGGGGCCTCTACCGGGGGATCGCCCCCA
ACTTCATGAAGGTTATTCCAGCTGTGAGCATCTCCTATGTGGTCTACGAGAACATGAAGCAGGCCTTG
GGGGTCACGTCCAGGTGAGGGACCCGGAGCCCGTCCCCCCAATCCCTCACCCCCC

| | | | |
|---|---|---|---|
| NOV12a, CG56262-01 | SEQ ID NO: 54 | 468 aa | MW at 52387.5kD |
| Protein Sequence | | | |

MRGSPGDAERRQRWGRLFEELDSNKDGRVDVHELRQGLARLGGGNPDPGAQQGISSEGDADPDGGLDL
EEFSRYLQEREQRLLLMFHSLDRNQDGHIDVSEIQQSFRALGISISLEQAEKILHSMDRDGTMTIDWQ
EWRDHFLLHSLENVEDVLYFWKHSTVLDIGECLTVPDEFSKQEKLTGMWWKQLVAGAVAGAVSRTGTA
PLDRLKVFIQVHASKTNRLNILGGLRSMVLEGGIRCLWRGNGINVLKIAPESAIKFMAYEQVRRAILG
QQETLHVQERFVAGSLAGATAQTIIYPMEVLKTRLTLRRTGQYKGLLDCARRILEREGPRAFYRGYLP
NVLGIIPYAGIDLAVYEVLKNWWLQQYSHDSADPGILVLLACGTISSTCGQIASYPLALVRTRMQAQA
SIEGGPQLSMLGLLRHILSQEGMRGLYRGIAPNFMKVIPAVSISYVVYENMKQALGVTSR

Further analysis of the NOV12a protein yielded the following properties shown in Table 12B.

TABLE 12B
Protein Sequence Properties NOV12a

| SignalP analysis: | No Known Signal Sequence Predicted | | | |
|---|---|---|---|---|
| PSORT II analysis: | PSG: a new signal peptide prediction method | | | |
| | N-region: length 11; pos. chg 3; neg. chg 2 | | | |
| | H-region: length 1; peak value −14.40 | | | |
| | PSG score: −18.80 | | | |
| | GvH: von Heijne's method for signal seq. recognition | | | |
| | GvH score (threshold: −2.1): −7.60 | | | |
| | possible cleavage site: between 46 and 47 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL Likelihood = 0.85 (at 342) | | | |
| | ALOM score: −0.90 (number of TMSs: 0) | | | |
| | MITDISC: discrimination of mitochondrial targeting seq | | | |
| | R content: | 1 | Hyd Moment(75): | 10.59 |
| | Hyd Moment(95): | 7.77 | G content: | 2 |
| | D/E content: | 2 | S/T content: | 1 |

```
Score: −5.97
Gavel: prediction of cleavage sites for mitochondrial preseq
R-2 motif at 12 MRG|SP
NUCDISC: discrimination of nuclear localization signals
pat4: none
pat7: none
bipartite: none
content of basic residues: 11.3%
NLS Score: −0.47
KDEL: ER retention motif in the C-terminus: none
ER Membrane Retention Signals:
XXRR-like motif in the N-terminus: RGSP
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs:
Leucine zipper pattern (PS00029): *** found ***
LEEFSRYLQEREQRLLLMFHSL at 68
none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
52.2%: cytoplasmic
30.4%: nuclear
  8.7%: mitochondrial
  4.3%: vacuolar
  4.3%: vesicles of secretory system
>> prediction for CG56262-01 is cyt (k = 23)
```

A search of the NOV12a protein against the Geneseq database, a proprietary database that contains sequences published in patents and patent publication, yielded several homologous proteins shown in Table 12C.

TABLE 12C
Geneseq Results for NOV12a

| | | | Identities/ | |
|---|---|---|---|---|
| | | NOV12a | Similarities | |
| | | Residues/ | for the | |
| Geneseq | Protein/Organism/Length | Match | Matched | Expect |
| Identifier | [Patent #, Date] | Residues | Region | Value |
| AAE22927 | Human transporter and ion | 1 . . . 468 | 463/468 (98%) | 0.0 |
| | channel (TRICH) 26 - Homo | 1 . . . 468 | 466/468 (98%) | |
| | sapiens, 468 aa. [WO200222684- | | | |
| | A2, 21 MAR. 2002] | | | |
| AAU27869 | Human contig polypeptide | 1 . . . 468 | 463/468 (98%) | 0.0 |
| | sequence #22 - Homo sapiens, 509 | 39 . . . 506 | 466/468 (98%) | |
| | aa. [WO200164834-A2, 07 SEP. | | | |
| | 2001] | | | |
| AAU27697 | Human full-length polypeptide | 1 . . . 468 | 463/468 (98%) | 0.0 |
| | sequence #22 - Homo sapiens, 471 | 1 . . . 468 | 466/468 (98%) | |
| | aa. [WO200164834-A2, 07 SEP. | | | |
| | 2001] | | | |
| ABG22637 | Novel human diagnostic protein | 1 . . . 468 | 442/470 (94%) | 0.0 |
| | #22628 - Homo sapiens, 508 aa. | 39 . . . 508 | 450/470 (95%) | |
| | [WO200175067-A2, 11 OCT. | | | |
| | 2001] | | | |
| ABG30434 | Human protein sequence #2 used | 1 . . . 409 | 403/409 (98%) | 0.0 |
| | for determining sequence of | 1 . . . 409 | 406/409 (98%) | |
| | unknown gene - Homo sapiens, | | | |
| | 456 aa. [JP2002176980-A, 25 | | | |
| | JUN. 2002] | | | |

In a BLAST search of public sequence databases, the NOV12a protein was found to have homology to the proteins shown in the BLASTP data in Table 12D.

TABLE 12D
Public BLASTP Results for NOV12a

| | | NOV12a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAD55563 | Putative calcium binding | 1 . . . 407 | 402/407 (98%) | 0.0 |
| | transporter - Homo sapiens | 1 . . . 407 | 405/407 (98%) | |
| | (Human), 438 aa. | | | |
| Q96NQ4 | Hypothetical protein FLJ30339 - | 85 . . . 468 | 379/384 (98%) | 0.0 |
| | Homo sapiens (Human), 384 aa. | 1 . . . 384 | 382/384 (98%) | |
| CAD20531 | Sequence 1 from Patent | 1 . . . 407 | 374/407 (91%) | 0.0 |
| | WO0174854 - Homo sapiens | 1 . . . 406 | 388/407 (94%) | |
| | (Human), 460 aa (fragment). | | | |
| AAH43834 | Similar to hypothetical protein | 6 . . . 468 | 302/463 (65%) | e−177 |
| | MGC36388 - Xenopus laevis | 53 . . . 514 | 368/463 (79%) | |
| | (African clawed frog), 514 aa. | | | |
| Q8BHG0 | Weakly similar to peroxisomal | 6 . . . 468 | 305/463 (65%) | e−176 |
| | CA-dependent solute carrier - | 41 . . . 502 | 366/463 (78%) | |
| | Mus musculus (Mouse), 502 aa. | | | |

PFam analysis predicts that the NOV12a protein contains the domains shown in the Table 12E.

TABLE 12E
Domain Analysis of NOV12a

| | | Identities/ | |
|---|---|---|---|
| | NOV12a | Similarities | |
| Pfam | Match | for the | Expect |
| Domain | Region | Matched Region | Value |
| efhand | 13 . . . 41 | 8/29 (28%) | 0.00022 |
| | | 26/29 (90%) | |
| efhand | 81 . . . 109 | 8/29 (28%) | 0.012 |
| | | 21/29 (72%) | |
| mito_carr | 184 . . . 276 | 38/124 (31%) | 2.1e−24 |
| | | 78/124 (63%) | |
| mito_carr | 278 . . . 369 | 38/124 (31%) | 3.4e−32 |
| | | 81/124 (65%) | |
| mito_carr | 375 . . . 468 | 31/124 (25%) | 2.4e−23 |
| | | 76/124 (61%) | |

### Example 13

The NOV13 clone was analyzed, and the nucleotide and encoded polypeptide sequences are shown in Table 13A.

TABLE 13A
NOV13 Sequence Analysis

| | | |
|---|---|---|
| NOV13a, CG56829-01 | SEQ ID NO: 55 | 953 bp |
| DNA Sequence | ORF Start: ATG at 50 | ORF Stop: end of sequence |

CAGAGGCAGCATGAGCTGAGAGGGTGATAGGAAGGCGGCGCTAGACAGCATGGAGGACTTTCTGCTCT
CCAATGGGTACCAGCTGGGCAAGACCATTGGGGAAGGGACCTACTCAAAAGTCAAAGAAGCATTTTCC
AAAAAACACCAAAGAAAAGTGGCAATTAAAGTTATAGACAAGATGGGAACTTCCTCAGAGTTTATCCA
GAGATTCCTCCCTCGGGAGCTCCAAATCGTCCGTACCCTGGACCACAAGAACATCATCCAGGTGTATG
AGATGCTGGAGTCTGCCGACGGGAAAATCTGCCTGGTGATGGAGCTCGCTGAGGGAGGGGATGTCTTT
GACTGCGTGCTGAATGGGGGGCCACTGCCTGAAAGCCGGGCCAAGGCCCTCTTCCGTCAGATGGTTGA
GGCCATCCGCTACTGCCATGGCTGTGGTGTGGCCCACCGGGACCTCAAATGTGAGAACGCCTTGTTGC
AGGGCTTCAACCTGAAGCTGACTGACTTTGGCTTTGCCAAGGTGTTGCCCAAGTCACACCGGGAGCTG
AGCCAGACCTTCTGCGGCAGTACAGCCTATGCTGCCCCCGAGGTGCTGCAGGGCATTCCCCACGATAG
CAAAAAAGGTGATGTCTGGAGCATGGGTGTGGTCCTGTATGTCATGCTCTGTGCCAGCCTACCTTTTG
ACGACACAGACATCCCCAAGATGCTGTGGCAGCAGCAGAAGGGGGTGTCCTTCCCCACTCATCTGAGC
ATCTCGGCCGATTGCCAGGACCTGCTCAAGAGGCTCCTGGAACCCGATATGATCCTCCGGCCTTCAAT
TGAAGAAGTTAGTTGGCATCCATGGCTAGCAAGCACTTGATAAAAGCAATGGCAAGTGCTCTCCAATA
AAGTAGGGGGAGAAAGCAAACCCAAAAACCCGCTTCTAAAATGGTGATATATATTTTACGCTTTAAGT
T

| | | | |
|---|---|---|---|
| NOV13a, CG56829-01 | SEQ ID NO: 56 | 268 aa | MW at 30093.6kD |
| Protein Sequence | | | |

MEDFLLSNGYQLGKTIGEGTYSKVKEAFSKKHQRKVAIKVIDKMGTSSEFIQRFLPRELQIVRTLDHK
NIIQVYEMLESADGKICLVMELAEGGDVFDCVLNGGPLPESRAKALFRQMVEAIRYCHGCGVAHRDLK
CENALLQGFNLKLTDFGFAKVLPKSHRELSQTFCGSTAYAAPEVLQGIPHDSKKGDVWSMGVVLYVML
CASLPFDDTDIPKMLWQQQKGVSFPTHLSISADCQDLLKRLLEPDMILRPSIEEVSWHPWLAST

Further analysis of the NOV13a protein yielded the following properties shown in Table 13B.

TABLE 13B
Protein Sequence Properties NOV13a

| SignalP analysis: | No Known Signal Sequence Predicted | | | |
|---|---|---|---|---|
| PSORT II analysis: | PSG: a new signal peptide prediction method | | | |
| | N-region: length 3; pos.chg 0; neg.chg 2 | | | |
| | H-region: length 10; peak value 0.00 | | | |
| | PSG score: −4.40 | | | |
| | GvH: von Heijne's method for signal seq. recognition | | | |
| | GvH score (threshold: −2.1): −9.40 | | | |
| | possible cleavage site: between 17 and 18 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL Likelihood = 2.92 (at 84) | | | |
| | ALOM score: −1.91 (number of TMSs: 0) | | | |
| | MITDISC: discrimination of mitochondrial targeting seq | | | |
| | R content: | 0 | Hyd Moment (75): | 8.14 |
| | Hyd Moment(95): | 7.70 | G content: | 0 |
| | D/E content: | 2 | S/T content: | 0 |

Score: −6.58
Gavel: prediction of cleavage sites for mitochondrial preseq
cleavage site motif not found
NUCDISC: discrimination of nuclear localization signals
pat4: none
pat7: none
bipartite: none
content of basic residues: 11.6%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
52.2%: cytoplasmic
30.4%: mitochondrial
17.4%: nuclear
>> prediction for CG56829-01 is cyt (k = 23)

A search of the NOV13a protein against the Geneseq database, a proprietary database that contains sequences published in patents and patent publication, yielded several homologous proteins shown in Table 13C.

TABLE 13C
Geneseq Results for NOV13a

| | | NOV13a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB65682 | Novel protein kinase, SEQ ID | 1 . . . 268 | 266/268 (99%) | e−155 |
| | NO: 210 - Homo sapiens, 268 aa. | 1 . . . 268 | 266/268 (99%) | |
| | [WO200073469-A2, 07 DEC. | | | |
| | 2000] | | | |
| AAM47999 | Human hTSSK3 SEQ ID NO 2 - | 1 . . . 268 | 265/268 (98%) | e−155 |
| | Homo sapiens, 268 aa. | 1 . . . 268 | 265/268 (98%) | |
| | [WO200185954-A2, 15 NOV. | | | |
| | 2001] | | | |
| AAE19154 | Human kinase polypeptide | 1 . . . 268 | 265/268 (98%) | e−155 |
| | (PKIN-12) - Homo sapiens, 268 | 1 . . . 268 | 265/268 (98%) | |
| | aa. [WO200208399-A2, 31 JAN. | | | |
| | 2002] | | | |
| ABG30417 | Human testis specific kinase | 1 . . . 268 | 265/268 (98%) | e−155 |
| | (TSSK) 3 protein - Homo | 1 . . . 268 | 265/268 (98%) | |
| | sapiens, 268 aa. [WO200238732- | | | |
| | A2, 16 MAY 2002] | | | |
| ABG30419 | Mouse testis specific kinase | 1 . . . 268 | 259/268 (96%) | e−151 |
| | (TSSK) 3b protein - Mus sp, 268 | 1 . . . 268 | 261/268 (96%) | |
| | aa. [WO200238732-A2, 16 | | | |
| | MAY 2002] | | | |

In a BLAST search of public sequence databases, the NOV13a protein was found to have homology to the proteins shown in the BLASTP data in Table 13D.

TABLE 13D
Public BLASTP Results for NOV13a

| | | NOV13a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96PN8 | Testis-specific serine/threonine | 1 . . . 268 | 265/268 (98%) | e−154 |
| | kinase 3 (Similar to serine/ | 1 . . . 268 | 265/268 (98%) | |
| | threonine kinase) (TSSK3) - | | | |
| | Homo sapiens (Human), 268 aa. | | | |
| Q9D2E1 | 4930594I21Rik protein (Testis- | 1 . . . 268 | 259/268 (96%) | e−151 |
| | specific serine/threonine kinase | 1 . . . 268 | 261/268 (96%) | |
| | 3b) - Mus musculus (Mouse), 268 | | | |
| | aa. | | | |
| Q9JL98 | Testis specific serine kinase-3 - | 1 . . . 268 | 240/273 (87%) | e−134 |
| | Mus musculus (Mouse), 266 aa. | 1 . . . 266 | 245/273 (88%) | |
| Q8IY55 | Hypothetical protein - Homo | 5 . . . 265 | 125/266 (46%) | 1e−69 |
| | sapiens (Human), 358 aa. | 7 . . . 272 | 190/266 (70%) | |
| Q96PF2 | Testis specific serine/threonine | 5 . . . 265 | 125/266 (46%) | 1e−69 |
| | kinase 2 - Homo sapiens (Human), | 1 . . . 272 | 190/266 (70%) | |
| | 358 aa. | | | |

PFam analysis predicts that the NOV13a protein contains the domains shown in the Table 13E.

TABLE 13E
Domain Analysis of NOV13a

| | | Identities/ | |
|---|---|---|---|
| | NOV13a | Similarities for | |
| Pfam | Match | the Matched | Expect |
| Domain | Region | Region | Value |
| pkinase | 10 . . . 265 | 99/299 (33%) | 1.5e−80 |
| | | 203/299 (68%) | |

### Example 14

The NOV14 clone was analyzed, and the nucleotide and encoded polypeptide sequences are shown in Table 14A.

TABLE 14A
NOV14 Sequence Analysis

| | | |
|---|---|---|
| NOV14a, CG57183-01 | SEQ ID NO: 57 | 2490 bp |
| DNA Sequence | ORF Start: ATG at 40 | ORF Stop: end of sequence |

CGCGCGCTGCCTGAGGACGCCGCGGCCCCCGCCCCCGCCATGGGCGCCCCTGCCTGCGCCCTCGCGCT
CTGCGTGGCCGTGGCCATCGTGGCCGGCGCCTCCTCGGAGTCCTTGGGGACGGAGCAGCGCGTCGTGG
GGCGAGCGGCAGAAGTCCCGGGCCCAGAGCCCGGCCAGCAGGAGCAGTTGGTCTTCGGCAGCGGGGAT
GCTGTGGAGCTGAGCTGTCCCCCGCCCGGGGGTGGTCCCATGGGGCCCACTGTCTGGGTCAAGGATGG
CACAGGGCTGGTGCCCTCGGAGCGTGTCCTGGTGGGGCCCCAGCGGCTGCAGGTGCTGAATGCCTCCC
ACGAGGACTCCGGGGCCTACAGCTGCCGGCAGCGGCTCACGCAGCGCGTACTGTGCCACTTCAGTGTG
CGGGTGACAGACGCTCCATCCTCGGGAGATGACGAAGACGGGGAGGACGAGGCTGAGGACACAGGTGT
GGACACAGGGGCCCCTTACTGGACACGGCCCGAGCGGATGGACAAGAAGCTGCTGGCCGTGCCGGCCG
CCAACACCGTCCGCTTCCGCTGCCCAGCCGCTGGCAACCCCACTCCCTCCATCTCCTGGCTGAAGAAC
GGCAGGGAGTTCCGCGGCGAGCACCGCATTGGAGGCATCAAGCTGCGGCATCAGCAGTGGAGCCTGGT
CATGGAAAGCGTGGTGCCCTCGGACCGCGGCAACTACACCTGCGTCGTGGAGAACAAGTTTGGCAGCA
TCCGGCAGACGTACACGCTGGACGTGCTGGAGCGCTCCCCGCACCGGCCCATCCTGCAGGCGGGGCTG
CCGGCCAACCAGACGGCGGTGCTGGGCAGCGACGTGGAGTTCCACTGCAAGGTGTACAGTGACGCACA
GCCCCACATCCAGTGGCTCAAGCACGTGGAGGTGAACGGCAGCAAGGTGGGCCCGGACGGCACACCCT
ACGTTACCGTGCTCAAGACGGCGGGCGCTAACACCACCGACAAGGAGCTAGAGGTTCTCTCCTTGCAC
AACGTCACCTTTGAGGACGCCGGGGAGTACACCTGCCTGGCGGGCAATTCTATTGGGTTTTCTCATCA
CTCTGCGTGGCTGGTGGTGCTGCCAGCCGAGGAGGAGCTGGTGGAGGCTGACGAGGCGGGCAGTGTGT
ATGCAGGCATCCTCAGCTACGGGGTGGGCTTCTTCCTGTTCATCCTGGTGGTGGCGGCTGTGACGCTC
TGCCGCCTGCGCAGCCCCCCCAAGAAAGGCCTGGGCTCCCCCACCGTGCACAAGATCTCCCGCTTCCC
GCTCAAGCGACAGGTGTCCCTGGAGTCCAACGCGTCCATGAGCTCCAACACACCACTGGTGCGCATCG
CAAGGCTGTCCTCAGGGGAGGGCCCCACGCTGGCCAATGTCTCCGAGCTCGAGCTGCCTGCCGACCCC
AAATGGGAGCTGTCTCGGGCCCGGCTGACCCTGGGCAAGCCCCTTGGGGAGGGCTGCTTCGGCCAGGT
GGTCATGGCGGAGGCCATCGGCATTGACAAGGACCGGGCCGCCAAGCCTGTCACCGTAGCCGTGAAGA
TGCTGAAAGACGATGCCACTGACAAGGACCTGTCGGACCTGGTGTCTGAGATGGAGATGATGAAGATG
ATCGGGAAACACAAAAACATCATCAACCTGCTGGGCGCCTGCACGCAGGGCGGGCCCCTGTACGTGCT
GGTGGAGTACGCGGCCAAGGGTAACCTGCGGGAGTTTCTGCGGGCGCGGCGGCCCCCGGGCCTGGACT
ACTCCTTCGACACCTGCAAGCCGCCCGAGGAGCAGCTCACCTTCAAGGACCTGGTGTCCTGTGCCTAC
CAGGTGGCCCGGGGCATGGAGTACTTGGCCTCCCAGAAGTGCATCCACAGGGACCTGGCTGCCCGCAA
TGTGCTGGTGACCGAGGACAACGTGATGAAGATCGCAGACTTCGGGCTGGCCCGGGACGTGCACAACC
TCGACTACTACAAGAAGACAACCAACGGCCGGCTGCCCGTGAAGTGGATGGCGCCTGAGGCCTTGTTT
GACCGAGTCTACACTCACCAGAGTGACGTCTGGTCCTTTGGGGTCCTGCTCTGGGAGATCTTCACGCT
GGGGGGCTCCCCGTACCCCGGCATCCCTGTGGAGGAGCTCTTCAAGCTGCTGAAGGAGGGCCACCGCA
TGGACAAGCCCGCCAACTGCACACACGACCTGTACATGATCATGCGGGAGTGCTGGCATGCCGCGCCC
TCCCAGAGGCCCACCTTCAAGCAGCTGGTGGAGGACCTGGACCGTGTCCTTACCGTGACGTCCACCGA
CGAGTACCTGGACCTGTCGGCGCCTTTCGAGCAGTACTCCCCGGGTGGCCAGGACACCCCCAGCTCCA
GCTCCTCAGGGGACGACTCCGTGTTTGCCCACGACCTGCTGCCCCCGGCCCCACCCAGCAGTGGGGGC
TCGCGGACGTGAAGGGCCACTGGTCCCCAACAATGTGAGGGG

| | | | |
|---|---|---|---|
| NOV14a, CG57183-01 | SEQ ID NO: 58 | 806 aa | MW at 87708.7 kD |
| Protein Sequence | | | |

MGAPACALALCVAVAIVAGASSESLGTEQRVVGRAAEVPGPEPGQQEQLVFGSGDAVELSCPPPGGGP
MGPTVWVKDGTGLVPSERVLVGPQRLQVLNASHEDSGAYSCRQRLTQRVLCHFSVRVTDAPSSGDDED
GEDEAEDTGVDTGAPYWTRPERMDKKLLAVPAANTVRFRCPAAGNPTPSISWLKNGREFRGEHRIGGI
KLRHQQWSLVMESVVPSDRGNYTCVVENKFGSIRQTYTLDVLERSPHRPILQAGLPANQTAVLGSDVE
FHCKVYSDAQPHIQWLKHVEVNGSKVGPDGTPYVTVLKTAGANTTDKELEVLSLHNVTFEDAGEYTCL
AGNSIGFSHHSAWLVVLPAEEELVEADEAGSVYAGILSYGVGFFLFILVVAAVTLCRLRSPPKKGLGS
PTVHKISRFPLKRQVSLESNASMSSNTPLVRIARLSSGEGPTLANVSELELPADPKWELSRARLTLGK
PLGEGCFGQVVMAEAIGIDKDRAAKPVTVAVKMLKDDATDKDLSDLVSEMEMMKMIGKHKNIINLLGA
CTQGGPLYVLVEYAAKGNLREFLRARRPPGLDYSFDTCKPPEEQLTFKDLVSCAYQVARGMEYLASQK
CIHRDLAARNVLVTEDNVMKIADFGLARDVHNLDYYKKTTNGRLPVKWMAPEALFDRVYTHQSDVWSF
GVLLWEIFTLGGSPYPGIPVEELFKLLKEGHRMDKPANCTHDLYMIMRECWHAAPSQRPTFKQLVEDL
DRVLTVTSTDEYLDLSAPFEQYSPGGQDTPSSSSSGDDSVFAHDLLPPAPPSSGGSRT

| | | |
|---|---|---|
| NOV14b, CG57183-02 | SEQ ID NO: 59 | 2427 bp |
| DNA Sequence | ORF Start: ATG at 1 | ORF Stop: end of sequence |

ATGGGCGCCCCTGCCTGCGCCCTCGCGCTCTGCGTGGCCGTGGCCATCGTGGCCGGCGCCTCCTCGGA
GTCCTTGGGGACGGAGCAGCGCGTCGTGGGGCGAGCGGCAGAAGTCCCGGGCCCAGAGCCCGGCCAGC
AGGAGCAGTTGGTCTTCGGCAGCGGGGATGCTGTGGAGCTGAGCTGTCCCCCGCCCGGGGGTGGTCCC
ATGGGGCCCACTGTCTGGGTCAAGGATGGCACAGGGCTGGTGCCCTCGGAGCGTGTCCTGGTGGGGCC
CCAGCGGCTGCAGGTGCTGAATGCCTCCCACGAGGACTCCGGGGCCTACAGCTGCCGGCAGCGGCTCA
CGCAGCGCGTACTGTGCCACTTCAGTGTGCGGGTGACAGACGCTCCATCCTCGGGAGATGACGAAGAC
GGGGAGGACGAGGCTGAGGACACAGGTGTGGACACAGGGGCCCCTTACTGGACACGGCCCGAGCGGAT
GGACAAGAAGCTGCTGGCCGTGCCGGCCGCCAACACCGTCCGCTTCCGCTGCCCAGCCGCTGGCAACC
CCACTCCCTCCATCTCCTGGCTGAAGAACGGCAGGGAGTTCCGCGGCGAGCACCGCATTGGAGGCATC
AAGCTGCGGCATCAGCAGTGGAGCCTGGTCATGGAAAGCGTGGTGCCCTCGGACCGCGGCAACTACAC
CTGCGTCGTGGAGAACAAGTTTGGCAGCATCCGGCAGACGTACACGCTGGACGTGCTGGAGCGCTCCC
CGCACCGGCCCATCCTGCAGGCGGGGCTGCCGGCCAACCAGACGGCGGTGCTGGGCAGCGACGTGGAG
TTCCACTGCAAGGTGTACAGTGACGCACAGCCCCACATCCAGTGGCTCAAGCACGTGGAGGTGAACGG
CAGCAAGGTGGGCCCGGACGGCACACCCTACGTTACCGTGCTCAAGTCCTGGATCAGTGAGAGTGTGG
AGGCCGACGTGCGCCTCCGCCTGGCCAATGTGTCGGAGCGGGACGGGGGCGAGTACCTCTGTCGAGCC
ACCAATTTCATAGGCGTGGCCGAGAAGGCCTTTTGGCTGAGCGTTCACGGGCCCCGAGCAGCCGAGGA
GGAGCTGGTGGAGGCTGACGAGGCGGGCAGTGTGTATGCAGGCATCCTCAGCTACGGGGTGGGCTTCT
TCCTGTTCATCCTGGTGGTGGCGGCTGTGACGCTCTGCCGCCTGCGCAGCCCCCCCAAGAAAGGCCTG
GGCTCCCCCACCGTGCACAAGATCTCCCGCTTCCCGCTCAAGCGACAGGTGTCCCTGGAGTCCAACGC
GTCCATGAGCTCCAACACACCACTGGTGCGCATCGCAAGGCTGTCCTCAGGGGAGGGCCCCACGCTGG
CCAATGTCTCCGAGCTCGAGCTGCCTGCCGACCCCAAATGGGAGCTGTCTCGGGCCCGGCTGACCCTG
GGCAAGCCCCTTGGGGAGGGCTGCTTCGGCCAGGTGGTCATGGCGGAGGCCATCGGCATTGACAAGGA
CCGGGCCGCCAAGCCTGTCACCGTAGCCGTGAAGATGCTGAAAGACGATGCCACTGACAAGGACCTGT
CGGACCTGGTGTCTGAGATGGAGATGATGAAGATGATCGGGAAACACAAAAACATCATCAACCTGCTG
GGCGCCTGCACGCAGGGCGGGCCCCTGTACGTGCTGGTGGAGTACGCGGCCAAGGGTAACCTGCGGGA
GTTTCTGCGGGCGCGGCGGCCCCCGGGCCTGGACTACTCCTTCGACACCTGCAAGCCGCCCGAGGAGC
AGCTCACCTTCAAGGACCTGGTGTCCTGTGCCTACCAGGTGGCCCGGGGCATGGAGTACTTGGCCTCC
CAGAAGTGCATCCACAGGGACCTGGCTGCCCGCAATGTGCTGGTGACCGAGGACAACGTGATGAAGAT
CGCAGACTTCGGGCTGGCCCGGGACGTGCACAACCTCGACTACTACAAGAAGACAACCAACGGCCGGC
TGCCCGTGAAGTGGATGGCGCCTGAGGCCTTGTTTGACCGAGTCTACACTCACCAGAGTGACGTCTGG
TCCTTTGGGGTCCTGCTCTGGGAGATCTTCACGCTGGGGGGCTCCCCGTACCCCGGCATCCCTGTGGA
GGAGCTCTTCAAGCTGCTGAAGGAGGGCCACCGCATGGACAAGCCCGCCAACTGCACACACGACCTGT
ACATGATCATGCGGGAGTGCTGGCATGCCGCGCCCTCCCAGAGGCCCACCTTCAAGCAGCTGGTGGAG
GACCTGGACCGTGTCCTTACCGTGACGTCCACCGACGAGTACCTGGACCTGTCGGCGCCTTTCGAGCA
GTACTCCCCGGGTGGCCAGGACACCCCCAGCTCCAGCTCCTCAGGGGACGACTCCGTGTTTGCCCACG
ACCTGCTGCCCCCGGCCCCACCCAGCAGTGGGGGCTCGCGGACGTGA

| | | | |
|---|---|---|---|
| NOV14b, CG57183-02 | SEQ ID NO: 60 | 808 aa | MW at 88156.3 kD |
| Protein Sequence | | | |

MGAPACALALCVAVAIVAGASSESLGTEQRVVGRAAEVPGPEPGQQEQLVFGSGDAVELSCPPPGGGP
MGPTVWVKDGTGLVPSERVLVGPQRLQVLNASHEDSGAYSCRQRLTQRVLCHFSVRVTDAPSSGDDED
GEDEAEDTGVDTGAPYWTRPERMDKKLLAVPAANTVRFRCPAAGNPTPSISWLKNGREFRGEHRTGGI
KLRHQQWSLVMESVVPSDRGNYTCVVENKFGSIRQTYTLDVLERSPHRPILQAGLPANQTAVLGSDVE
FHCKVYSDAQPHIQWLKHVEVNGSKVGPDGTPYVTVLKSWISESVEADVRLRLANVSERDGGEYLCRA
TNFIGVAEKAFWLSVHGPRAAEEELVEADEAGSVYAGILSYGVGFFLFILVVAAVTLCRLRSPPKKGL
GSPTVHKISRFPLKRQVSLESNASMSSNTPLVRIARLSSGEGPTLANVSELELPADPKWELSRARLTL
GKPLGEGCFGQVVMAEAIGIDKDRAAKPVTVAVKMLKDDATDKDLSDLVSEMEMMKMIGKHKNIINLL
GACTQGGPLYVLVEYAAKGNLREFLRARRPPGLDYSFDTCKPPEEQLTFKDLVSCAYQVARGMEYLAS
QKCIHRDLAARNVLVTEDNVMKIADFGLARDVHNLDYYKKTTNGRLPVKWMAPEALFDRVYTHQSDVW
SFGVLLWEIFTLGGSPYPGIPVEELFKLLKEGHRMDKPANCTHDLYMIMRECWHAAPSQRPTFKQLVE
DLDRVLTVTSTDEYLDLSAPFEQYSPGGQDTPSSSSSGDDSVFAHDLLPPAPPSSGGSRT

A ClustalW comparison of the above protein sequences yields the following sequence alignment shown in Table 14B.

TABLE 14B
Comparison of the NOV14 protein sequences.

| | |
|---|---|
| NOV14a | MGAPACALALCVAVAIVAGASSESLGTEQRVVGRAAEVPGPEPGQQEQLVFGSGDAVELS |
| NOV14b | MGAPACALALCVAVAIVAGASSESLGTEQRVVGRAAEVPGPEPGQQEQLVFGSGDAVELS |
| NOV14a | CPPPGGGPMGPTVWVKDGTGLVPSERVLVGPQRLQVLNASHEDSGAYSCRQRLTQRVLCH |
| NOV14b | CPPPGGGPMGPTVWVKDGTGLVPSERVLVGPQRLQVLNASHEDSGAYSCRQRLTQRVLCH |
| NOV14a | FSVRVTDAPSSGDDEDGEDEAEDTGVDTGAPYWTRPERMDKKLLAVPAANTVRFRCPAAG |
| NOV14b | FSVRVTDAPSSGDDEDGEDEAEDTGVDTGAPYWTRPERMDKKLLAVPAANTVRFRCPAAG |
| NOV14a | NPTPSISWLKNGREFRGEHRIGGIKLRHQQWSLVMESVVPSDRGNYTCVVENKFGSIRQT |
| NOV14b | NPTPSISWLKNGREFRGEHRIGGIKLRHQQWSLVMESVVPSDRGNYTCVVENKFGSIRQT |
| NOV14a | YTLDVLERSPHRPILQAGLPANQTAVLGSDVEFHCKVYSDAQPHIQWLKHVEVNGSKVGP |
| NOV14b | YTLDVLERSPHRPILQAGLPANQTAVLGSDVEFHCKVYSDAQPHIQWLKHVEVNGSKVGP |
| NOV14a | DGTPYVTVLKTAGANTTDKELEVLSLHNVTFEDAGEYTCLAGNSIGFSHHSAWLVVLP-- |
| NOV14b | DGTPYVTVLKSWISESVEADVRLR-LANVSERDGGEYLCRATNFIGVAEKAFWLSVHGPR |
| NOV14a | -AEEELVEADEAGSVYAGILSYGVGFFLFILVVAAVTLCRLRSPPKKGLGSPTVHKISRF |
| NOV14b | AAEEELVEADEAGSVYAGILSYGVGFFLFILVVAAVTLCRLRSPPKKGLGSPTVHKISRF |
| NOV14a | PLKRQVSLESNASMSSNTPLVRIARLSSGEGPTLANVSELELPADPKWELSRARLTLGKP |
| NOV14b | PLKRQVSLESNASMSSNTPLVRIARLSSGEGPTLANVSELELPADPKWELSRARLTLGKP |
| NOV14a | LGEGCFGQVVMAEAIGIDKDRAAKPVTVAVKMLKDDATDKDLSDLVSEMEMMKMIGKHKN |
| NOV14b | LGEGCFGQVVMAEAIGIDKDRAAKPVTVAVKMLKDDATDKDLSDLVSEMEMMKMIGKHKN |
| NOV14a | IINLLGACTQGGPLYVLVEYAAKGNLREFLRARRPPGLDYSFDTCKPPEEQLTFKDLVSC |
| NOV14b | IINLLGACTQGGPLYVLVEYAAKGNLREFLRARRPPGLDYSFDTCKPPEEQLTFKDLVSC |
| NOV14a | AYQVARGMEYLASQKCIHRDLAARNVLVTEDNVMKIADFGLARDVHNLDYYKKTTNGRLP |
| NOV14b | AYQVARGMEYLASQKCIHRDLAARNVLVTEDNVMKIADFGLARDVHNLDYYKKTTNGRLP |
| NOV14a | VKWMAPEALFDRVYTHQSDVWSFGVLLWEIFTLGGSPYPGIPVEELFKLLKEGHRMDKPA |
| NOV14b | VKWMAPEALFDRVYTHQSDVWSFGVLLWEIFTLGGSPYPGIPVEELFKLLKEGHRMDKPA |
| NOV14a | NCTHDLYMIMRECWHAAPSQRPTFKQLVEDLDRVLTVTSTDEYLDLSAPFEQYSPGGQDT |
| NOV14b | NCTHDLYMIMRECWHAAPSQRPTFKQLVEDLDRVLTVTSTDEYLDLSAPFEQYSPGGQDT |
| NOV14a | PSSSSSGDDSVFAHDLLPPAPPSSGGSRT |
| NOV14b | PSSSSSGDDSVFAHDLLPPAPPSSGGSRT |
| NOV14a | (SEQ ID NO: 58) |
| NOV14b | (SEQ ID NO: 60) |

Further analysis of the NOV14a protein yielded the following properties shown in Table 14C.

TABLE 14C
Protein Sequence Properties NOV14a

| | |
|---|---|
| SignalP analysis: | Cleavage site between residues 23 and 24 |

PSORT II analysis:
PSG: a new signal peptide prediction method
N-region: length 0; pos. chg 0; neg. chg 0
H-region: length 22; peak value 9.90
PSG score: 5.50
GvH: von Heijne's method for signal seq. recognition
GvH score (threshold: −2.1): −0.62
possible cleavage site: between 22 and 23
>>> Seems to have a cleavable signal peptide (1 to 22)
ALOM: Klein et al's method for TM region allocation
Init position for calculation: 23
Tentative number of TMS(s) for the threshold 0.5: 1
Number of TMS(s) for threshold 0.5: 1

| | | | |
|---|---|---|---|
| INTEGRAL | Likelihood = | −9.98 | Transmembrane 380-396 |

| | | |
|---|---|---|
| PERIPHERAL | Likelihood = | 3.07 (at 677) |

ALOM score: −9.98 (number of TMSs: 1)
MTOP: Prediction of membrane topology (Hartmann et al.)
Center position for calculation: 11
Charge difference: −2.0 C(−1.0)-N( 1.0)
N >= C: N-terminal side will be inside
>>> membrane topology: type 1a (cytoplasmic tail 397 to 806)
MITDISC: discrimination of mitochondrial targeting seq

| | | | |
|---|---|---|---|
| R content: | 0 | Hyd Moment (75): | 2.17 |
| Hyd Moment (95): | 1.34 | G content: | 2 |
| D/E content: | 1 | S/T content: | 2 |

Score: −6.45
Gavel: prediction of cleavage sites for mitochondrial preseq
cleavage site motif not found
NUCDISC: discrimination of nuclear localization signals
pat4: none
pat7: none
bipartite: none
content of basic residues: 10.0%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: MGAPACA
Prenylation motif: none
memYQRL: transport motif from cell surface to Golgi: none
Tyrosines in the tail: too long tail
Dileucine motif in the tail: found
LL at 541
LL at 683
LL at 706
LL at 793
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 89
COIL: Lupas's algorithm to detect coiled-coil regions
total: 0 residues
Final Results (k = 9/23):
44.4%: endoplasmic reticulum
22.2%: Golgi
22.2%: extracellular, including cell wall
11.1%: plasma membrane
>> prediction for CG57183-01 is end (k = 9)

A search of the NOV14a protein against the Geneseq database, a proprietary database that contains sequences published in patents and patent publication, yielded several homologous proteins shown in Table 14D.

TABLE 14D
Geneseq Results for NOV14a

| | | NOV14a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE21976 | Human fibroblast growth factor | 1 . . . 806 | 805/806 (99%) | 0.0 |
| | receptor 3c (FGFR3c) mutant | 1 . . . 806 | 805/806 (99%) | |
| | protein - Homo sapiens, 806 aa. | | | |
| | [US6265632-B1, 24 JUL. 2001] | | | |
| AAE21977 | Mouse fibroblast growth factor | 1 . . . 806 | 745/807 (92%) | 0.0 |
| | receptor 3 (FGFR3) mutant | 1 . . . 801 | 763/807 (94%) | |
| | protein - Mus sp, 801 aa. | | | |
| | [US6265632-B1, 24 JUL. 2001] | | | |
| AAU02978 | Angiotensin converting enzyme | 1 . . . 542 | 541/542 (99%) | 0.0 |
| | (ACEV) splice variant protein | 1 . . . 542 | 541/542 (99%) | |
| | #78 - Homo sapiens, 561 aa. | | | |
| | [WO200136632-A2, 25 MAY | | | |
| | 2001] | | | |
| AAR21080 | flg receptor protein - Homo | 45 . . . 798 | 535/769 (69%) | 0.0 |
| | sapiens, 821 aa. [WO9200999-A, | 46 . . . 807 | 615/769 (79%) | |
| | 23 JAN. 1992] | | | |
| AAB84383 | Amino acid sequence of a | 39 . . . 798 | 508/774 (65%) | 0.0 |
| | fibroblast growth factor receptor - | 33 . . . 803 | 608/774 (77%) | |
| | Homo sapiens, 820 aa. | | | |
| | [US6255454-B1, 03 JUL. 2001] | | | |

In a BLAST search of public sequence databases, the NOV14a protein was found to have homology to the proteins shown in the BLASTP data in Table 14E.

TABLE 14E
Public BLASTP Results for NOV14a

| | | NOV14a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P22607 | Fibroblast growth factor receptor | 1 . . . 806 | 806/806 (100%) | 0.0 |
| | 3 precursor (EC 2.7.1.112) | 1 . . . 806 | 806/806 (100%) | |
| | (FGFR-3) - Homo sapiens | | | |
| | (Human), 806 aa. | | | |
| Q8NI15 | Fibroblast growth factor receptor | 38 . . . 806 | 769/769 (100%) | 0.0 |
| | 3 - Homo sapiens (Human), 769 | 1 . . . 769 | 769/769 (100%) | |
| | aa (fragment). | | | |
| A48991 | heparin-binding growth factor | 1 . . . 806 | 747/806 (92%) | 0.0 |
| | receptor - mouse, 800 aa. | 1 . . . 800 | 765/806 (94%) | |
| Q9JHX9 | Fibroblast growth factor receptor | 1 . . . 806 | 745/806 (92%) | 0.0 |
| | 3 - Rattus norvegicus (Rat), 800 | 1 . . . 800 | 764/806 (94%) | |
| | aa. | | | |
| Q99052 | Fibroblast growth factor | 1 . . . 806 | 745/806 (92%) | 0.0 |
| | receptor - Mus musculus | 1 . . . 800 | 764/806 (94%) | |
| | (Mouse), 800 aa. | | | |

PFam analysis predicts that the NOV14a protein contains the domains shown in the Table 14F.

TABLE 14F
Domain Analysis of NOV14a

| | | Identities/ | |
|---|---|---|---|
| | NOV14a | Similarities for | |
| Pfam | Match | the Matched | Expect |
| Domain | Region | Region | Value |
| ig | 54 . . . 111 | 18/59 (31%) | 8.3e−05 |
| | | 40/59 (68%) | |
| ig | 169 . . . 230 | 21/65 (32%) | 5.6e−07 |
| | | 50/65 (77%) | |
| ig | 268 . . . 341 | 18/77 (23%) | 1.6e−06 |
| | | 54/77 (70%) | |
| pkinase | 472 . . . 750 | 98/319 (31%) | 2.2e−91 |
| | | 237/319 (74%) | |

### Example 15

The NOV15 clone was analyzed, and the nucleotide and encoded polypeptide sequences are shown in Table 15A.

TABLE 15A
NOV15 Sequence Analysis

| | | |
|---|---|---|
| | SEQ ID NO: 61 | 2077 bp |
| NOV15a, CG57341-01 | SEQ ID NO: 61 | 2077 bp |
| DNA Sequence | | |

ATGGAGCGGTGGCGCGACCGG [symbol]
CCGGGCCCTGGTCCAGCAGGGACTGAAGGTGGTGGGCTGCGCCCGCACTGTGGGCAACATCGAGGAGC
TGGCTGCTGAATGTAAGAGTGCAGGCTACCCCGGGACTTTGATCCCCTACAGATGTGACCTATCAAAT
GAAGAGGACATCCTCTCCATGTTCTCAGCTATCCGTTCTCAGCACAGCGGTGTAGACATCTGCATCAA
CAATGCTGGCTTGGCCCGGCCTGACACCCTGCTCTCAGGCAGCACCAGTGGTTGGAAGGACATGTTCA
ATGTGAACGTGCTGGCCCTCAGCATCTGCACACGGGAAGCCTACCAGTCCATGAAGGAGCGGAATGTG
GACGATGGGCACATCATTAACATCAATAGCATGTCTGGCCACCGAGTGTTACCCCTGTCTGTGACCCA
CTTCTATAGTGCCACCAAGTATGCCGTCACTGCGCTGACAGAGGGACTGAGGCAAGAGCTTCGGGAGG
CCCAGACCCACATCCGAGCCACGTGGCAGCTTCGGAGGGAGGAGGCCGCTGCCGGATATCAGGCAGCC
ATCACTGTGAAGCTGGGGTTCTGTGGCCTCCATCCTCTCCCCTCGACCTCCCCAAGACCTGGCAAAGC
TCAGCCCCTGAGAAGGCCCTCTCTGTTGGCCCAGTGCATCTCTCCAGGTGTGGTGGAGACACAATTCG
CCTTCAAACTCCACGACAAGGACCCTGAGAAGGCAGCTGCCACCTATGAGCAAATGAAGTGTCTCAAA
CCCGAGGATGTGGCCGAGGCTGTTATCTACGTCCTCAGCACCCCCGCACACATCCAGATTGGAGACAT
CCAGATGAGGCCCACGGAGCAGAGAGCTCGGCGGAGACGGCTGTCGAGTACCCTTCACCTCGGTGTTG
GGAGCCTGGGAGCGAACTGCGGCGCGGGTTACCGCTCCCGGGGACGCAGCAAGGGGCATCGAGTCCCT
GGCGGGAGCTGCGCCATGGCATTGCTCTCGACCGTCCGGGGCGCGACCTGGGGTCGCCTCGTCACCCG
TCATTTCTCCCATGCAGCGCGGCATGGGGAGCGGCCTGGTGGGGAGGAGCTAAGCCGCTTGCTGCTGG
ATGACCTGGTGCCGACCTCTCGGCTGGAGCTTCTGTTTGGCATGACCCCGTGTCTCCTGGCTCTGCAG
GCCGCCCGCCGCTCTGTGGCCCGGCTCCTGCTCCAGGCGGGTAAAGCTGGGCTGCAGGGGAAGCGGGC
CGAGCTGCTCCGGATGGCCGAGGCGCGGGACATTCCAGTTCTGCGGCCCAGACGGCAGAAACTGGACA
CAATGTGCCGCTACCAGGTCCACCAGGGTGTCTGCATGGAGGTGAGCCCGCTGCGGCCCCGGCCTTGG
AGAGAGGCCGGGGAGGCGAGCCCAGGCGACGACCCCCAGCAGTTGTGGCTCGTCCTCGATGGGATCCA
GGATCCCCGGAATTTTGGGGCTGTGCTGCGTTCCGCACACTTCCTCGGAGTGGATAAGACCAAAGCCC
AGCAGGGCTGGCTCGTGGCCGGCACGGTGGGCTGCCCAAGCACAGAGGATCCCCAGTCCTCCGAGATC
CCCATCATGAGTTGCTTGGAGTTCCTCTGGGAACGGCCTACTCTCCTTGTGCTGGGGAATGAGGGCTC
AGGTCTATCCCAGGAGGTGCAGGCCTCCTGCCAGCTTCTCCTCACCATCCTGCCCCGGCGCCAGCTGC
CTCCTGGACTTGAGTCCTTGAACGTCTCTGTGGCTGCAGGAATTCTTCTTCACTCCATTTGCAGCCAG
AGGAAGGGTTTCCCCACAGAGGGGGAGAGAAGGCAGCTTCTCCAAGACCCCCAAGAACCCTCAGCCAG
GTCTGAAGGGCTCAGCATGGCTCAGCACCCAGGGCTGTCTTCAGGCCCAGAGAAAGAGAGGCAAAATG
AGGGCTGACGTGGACTGTCCACAGTGTTCATGTGCTGGAGTCAGGGACGGCCGCACCTGCCTCCGCCG
GCTCCAGTGTGCGGGGAGCCTCTGCCTGAGTGTGCAC

| | | | |
|---|---|---|---|
| NOV15a, CG57341-01 | SEQ ID NO: 62 | 659 aa | MW at 71854.5 kD |
| Protein Sequence | | | |

MERWRDRLALVTGASGGIGAAVARALVQQGLKVVGCARTVGNIEELAAECKSAGYPGTLIPYRCDLSN
EEDILSMFSAIRSQHSGVDICINNAGLARPDTLLSGSTSGWKDMFNVNVLALSICTREAYQSMKERNV
DDGHIININSMSGHRVLPLSVTHFYSATKYAVTALTEGLRQELREAQTHIRATWQLRREEAAAGYQAA
ITVKLGFCGLHPLPSTSPRPGKAQPLRRPSLLAQCISPGVVETQFAFKLHDKDPEKAAATYEQMKCLK
PEDVAEAVIYVLSTPAHIQIGDIQMRPTEQRARRRRLSSTLHLGVGSLGANCGAGYRSRGRSKGHRVP
GGSCAMALLSTVRGATWGRLVTRHFSHAARHGERPGGEELSRLLLDDLVPTSRLELLFGMTPCLLALQ
AARRSVARLLLQAGKAGLQGKRAELLRMAEARDIPVLRPRRQKLDTMCRYQVHQGVCMEVSPLRPRPW
REAGEASPGDDPQQLWLVLDGIQDPRNFGAVLRSAHFLGVDKTKAQQGWLVAGTVGCPSTEDPQSSEI
PIMSCLEFLWERPTLLVLGNEGSGLSQEVQASCQLLLTILPRRQLPPGLESLNVSVAAGILLHSICSQ
RKGFPTEGERRQLLQDPQEPSARSEGLSMAQHPGLSSGPEKERQNEG

Further analysis of the NOV15a protein yielded the following properties shown in Table 15B.

TABLE 15B
Protein Sequence Properties NOV15a

| | |
|---|---|
| SignalP analysis: | No Known Signal Sequence Predicted |

PSORT II analysis:
PSG: a new signal peptide prediction method
N-region: length 7; pos. chg 3; neg. chg 2
H-region: length 16; peak value 7.43
PSG score: 3.03
GvH: von Heijne's method for signal seq. recognition
GvH score (threshold: −2.1): −0.02
possible cleavage site: between 20 and 21
>>> Seems to have a cleavable signal peptide (1 to 20)
ALOM: Klein et al's method for TM region allocation
Init position for calculation: 21
Tentative number of TMS(s) for the threshold 0.5: 0
number of TMS(s) . . . fixed
PERIPHERAL Likelihood = 0.63 (at 394)
ALOM score: 0.63 (number of TMSs: 0)
MTOP: Prediction of membrane topology (Hartmann et al.)
Center position for calculation: 10
Charge difference: 1.0 C( 3.0)-N( 2.0)
C > N: C-terminal side will be inside
>>>Caution: Inconsistent mtop result with signal peptide
MITDISC: discrimination of mitochondrial targeting seq

| | | | |
|---|---|---|---|
| R content: | 2 | Hyd Moment (75): | 4.39 |
| Hyd Moment (95): | 11.67 | G content: | 0 |
| D/E content: | 2 | S/T content: | 0 |

```
Score: −4.81
Gavel: prediction of cleavage sites for mitochondrial preseq
cleavage site motif not found
NUCDISC: discrimination of nuclear localization signals
pat4: RRRR (5) at 305
pat4: RPRR (4) at 446
pat7: PVLRPRR (3) at 443
pat7: PRRQKLD (5) at 447
bipartite: none
content of basic residues: 12.0%
NLS Score: 0.78
KDEL: ER retention motif in the C-terminus: none
ER Membrane Retention Signals:
XXRR-like motif in the N-terminus: ERWR
none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 55.5
COIL: Lupas's algorithm to detect coiled-coil regions
total: 0 residues
Final Results (k = 9/23):
47.8%: nuclear
39.1%: mitochondrial
  4.3%: cytoplasmic
  4.3%: vacuolar
  4.3%: extracellular, including cell wall
>> prediction for CG57341-01 is nuc (k = 23)
```

A search of the NOV15a protein against the Geneseq database, a proprietary database that contains sequences published in patents and patent publication, yielded several homologous proteins shown in Table 15C.

TABLE 15C
Geneseq Results for NOV15a

| | | NOV15a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABU10698 | Human secreted/transmembrane | 1 . . . 302 | 253/302 (83%) | e−137 |
| | protein #189 - Homo sapiens, 260 | 6 . . . 258 | 253/302 (83%) | |
| | aa. [US2002127584-A1, 12 SEP. | | | |
| | 2002] | | | |
| AAU76220 | Human 21620 alcohol | 1 . . . 302 | 253/302 (83%) | e−137 |
| | dehydrogenase (ADH) protein - | 6 . . . 258 | 253/302 (83%) | |
| | Homo sapiens, 260 aa. | | | |
| | [US2002010946-A1, 24 JAN. | | | |
| | 2002] | | | |
| ABG95914 | Human secreted/transmembrane | 1 . . . 302 | 253/302 (83%) | e−137 |
| | protein PRO 1774 - Homo sapiens, | 6 . . . 258 | 253/302 (83%) | |
| | 260 aa. [US2002119130-A1, 29 | | | |
| | AUG. 2002.] | | | |
| AAB87589 | Human PRO1774 - Homo sapiens, | 1 . . . 302 | 253/302 (83%) | e−137 |
| | 260 aa. [WO200116318-A2, 08 | 6 . . . 258 | 253/302 (83%) | |
| | MAR. 2001] | | | |
| AAB84364 | Amino acid sequence of human | 1 . . . 302 | 253/302 (83%) | e−137 |
| | alcohol dehydrogenase 21620 - | 6 . . . 258 | 253/302 (83%) | |
| | Homo sapiens, 260 aa. | | | |
| | [WO200144446-A2, 21 JUN. | | | |
| | 2001] | | | |

In a BLAST search of public sequence databases, the NOV15a protein was found to have homology to the proteins shown in the BLASTP data in Table 15D.

TABLE 15D
Public BLASTP Results for NOV15a

| | | NOV15a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC43880 | Sequence 2 from Patent | 1 . . . 302 | 253/302 (83%) | e−136 |
| | WO0144446 - Homo sapiens | 6 . . . 258 | 253/302 (83%) | |
| | (Human), 260 aa. | | | |
| Q9BUC7 | Hypothetical protein - Homo | 75 . . . 302 | 179/228 (78%) | 5e−92 |
| | sapiens (Human), 181 aa. | 1 . . . 179 | 179/228 (78%) | |
| Q96GK2 | Hypothetical protein - Homo | 515 . . . 659 | 142/145 (97%) | 1e−76 |
| | sapiens (Human), 158 aa. | 14 . . . 158 | 143/145 (97%) | |
| Q9H664 | Hypothetical protein | 515 . . . 659 | 142/145 (97%) | 1e−76 |
| | FLJ22578 - Homo sapiens | 11 . . . 155 | 143/145 (97%) | |
| | (Human), 155 aa. | | | |
| Q8BRZ1 | Hypothetical protein - Mus | 357 . . . 523 | 131/167 (78%) | 6e−71 |
| | musculus (Mouse), 197 aa. | 10 . . . 176 | 144/167 (85%) | |

PFam analysis predicts that the NOV15a protein contains the domains shown in the Table 15E.

TABLE 15E
Domain Analysis of NOV15a

| | | Identities/ | |
|---|---|---|---|
| | | Similarities | |
| | NOV15a | for the | Expect |
| Pfam Domain | Match Region | Matched Region | Value |
| adh_short | 5 . . . 319 | 73/337 (22%) | 1.9e−33 |
| | | 217/337 (64%) | |
| SpoU_methylase | 490 . . . 607 | 37/156 (24%) | 1e−05 |
| | | 91/156 (58%) | |

### Example 16

The NOV16 clone was analyzed, and the nucleotide and encoded polypeptide sequences are shown in Table 16A.

TABLE 16A
NOV16 Sequence Analysis

| | | |
|---|---|---|
| NOV16a, CG57460-01 | SEQ ID NO: 63 | 736 bp |
| DNA Sequence | ORF Start: ATG at 13 | ORF Stop: end of sequence |

AAAGCACCCGAGATGACCCCGGCTCCTCCACCAGGAGCGCGGCCGGGCGCGGCGTCCCTAGCGGGCTT
CGCCGGGGTGGCGTCTCTGGGGCCTGGGGACCCCCGCCGCGCCGCTGACCCGCGCCCTCTGCCCCCAG
CGCTGTGCTTCGCCGTGAGCCGCTCGCTGCTGCTGACGTGCCTGGTGCCGGCCGCGCTGCTGGGCCTG
CGCTACTACTACAGCCGCAAGGTGATCCGCGCCTACCTGGAGTGCGCGCTGCACACGGACATGGCGGA
CATCGAGCAGTACTACATGAAGCCGCCCGGTGTGTCCCTGACCGCCCTATCCCCTGCAGGCTCCTGCT
TCTGGGTGGCCGTGCTGGATGGCAACGTGGTGGGCATTGTGGCTGCACGGGCCCACGAGGAGGACAAC
ACGGTGGAGCTGCTGCGGATGTCTGTGGACTCACGTTTCCGAGGCAAGGGCATCGCCAAGGCGCTGGG
CCGGAAGGTGCTGGAGTTCGCCGTGGTGCACAACTACTCCGCGGTGGTGCTGGGCACGACGGCCGTCA
AGGTGGCCGCCCACAAGCTCTACGAGTCGCTGGGCTTCAGACACATGGGCGCCAGTGACCACTACGTG
CTGCCGGGCATGACCCTCTCGCTGGCTGAGCGCCTCTTCTTCCAGGTCCGCTACCACCGCTACCGCCT
GCAGCTGCGCGAGGAGTGACCGCCGCCGCTCGCCCGCCCGCCCCCCCGGCCGCCCT

| | | | |
|---|---|---|---|
| NOV16a, CG57460-01 | SEQ ID NO: 64 | 228 aa | MW at 24767.5 kD |
| Protein Sequence | | | |

MTPAPPPGARPGAASLAGFAGVASLGPGDPRRAADPRPLPPALCFAVSRSLLLTCLVPAALLGLRYYY
SRKVIRAYLECALHTDMADIEQYYMKPPGVSLTALSPAGSCFWVAVLDGNVVGIVAARAHEEDNTVEL
LRMSVDSRFRGKGIAKALGRKVLEFAVVHNYSAVVLGTTAVKVAAHKLYESLGFRHMGASDHYVLPGM
TLSLAERLFFQVRYHRYRLQLREE

Further analysis of the NOV16a protein yielded the following properties shown in Table 16B.

TABLE 16B
Protein Sequence Properties NOV16a

| SignalP analysis: | Cleavage site between residues 64 and 65 | | | | |
|---|---|---|---|---|---|
| PSORT II analysis: | | | | | |
| | PSG: a new signal peptide prediction method | | | | |
| | N-region: length 10; pos. chg 1; neg. chg 0 | | | | |
| | H-region: length 18; peak value 8.32 | | | | |
| | PSG score: 3.92 | | | | |
| | GvH: von Heijne's method for signal seq. recognition | | | | |
| | GvH score (threshold: −2.1): −3.74 | | | | |
| | possible cleavage site: between 25 and 26 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 2 | | | | |
| | INTEGRAL | Likelihood = | −2.02 | Transmembrane | 46-62 |
| | INTEGRAL | Likelihood = | −2.34 | Transmembrane | 109-125 |

| PERIPHERAL | Likelihood = | 2.38 (at 161) | |
|---|---|---|---|
| ALOM score: −2.34 (number of TMSs: 2) | | | |
| MTOP: Prediction of membrane topology (Hartmann et al.) | | | |
| Center position for calculation: 53 | | | |
| Charge difference: 2.0 C(3.0)-N(1.O) | | | |
| C > N: C-terminal side will be inside | | | |
| >>>Caution: Inconsistent mtop result with signal peptide | | | |
| >>> membrane topology: type 3b | | | |
| MITDISC: discrimination of mitochondrial targeting seq | | | |
| R content: | 1 | Hyd Moment(75): | 0.78 |
| Hyd Moment (95): | 2.47 | G content: | 6 |
| D/E content: | 1 | S/T content: | 3 |

| Score: −7.20 | |
|---|---|
| Gavel: prediction of cleavage sites for mitochondrial preseq | |
| R-2 motif at 20 ARP\|GA | |
| NUCDISC: discrimination of nuclear localization signals | |
| pat4: none | |
| pat7: none | |
| bipartite: none | |
| content of basic residues: 11.4% | |
| NLS Score: −0.47 | |
| KDEL: ER retention motif in the C-terminus: none | |
| ER Membrane Retention Signals: none | |
| SKL: peroxisomal targeting signal in the C-terminus: none | |
| PTS2: 2nd peroxisomal targeting signal: none | |
| VAC: possible vacuolar targeting motif: none | |
| RNA-binding motif: none | |
| Actinin-type actin-binding motif: | |
| type 1: none | |
| type 2: none | |
| NMYR: N-myristoylation pattern: none | |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: Reinhardt's method for Cytoplasmic/Nuclear discrimination | |
| Prediction: cytoplasmic | |
| Reliability: 94.1 | |
| COIL: Lupas's algorithm to detect coiled-coil regions | |
| total: 0 residues | |
| Final Results (k = 9/23): | |
| 44.4%: | endoplasmic reticulum |
| 11.1%: | vacuolar |
| 11.1%: | Golgi |
| 11.1%: | mitochondrial |
| 11.1%: | nuclear |
| 11.1%: | cytoplasmic |

>> prediction for CG57460-01 is end (k=9)

A search of the NOV16a protein against the Geneseq database, a proprietary database that contains sequences published in patents and patent publication, yielded several homologous proteins shown in Table 16C.

TABLE 16C
Geneseq Results for NOV16a

| | | | Identities/ | |
|---|---|---|---|---|
| | | NOV16a | Similarities | |
| | Protein/Organism/ | Residues/ | for the | |
| Geneseq | Length | Match | Matched | Expect |
| Identifier | [Patent #, Date] | Residues | Region | Value |
| AAE15442 | Human drug metabolising | 1 . . . 228 | 218/228 (95%) | e−121 |
| | enzyme (DME)-9 - Homo | 1 . . . 218 | 218/228 (95%) | |
| | sapiens, 218 aa. | | | |
| | [WO200179468- | | | |
| | A2, 25 OCT. 2001] | | | |
| ABU11545 | Human MDDT polypeptide SEQ | 42 . . . 228 | 176/187 (94%) | 2e−95 |
| | ID 492 - Homo | 20 . . . 196 | 177/187 (94%) | |
| | sapiens, 196 aa. | | | |
| | [WO200279449-A2, 10 OCT. | | | |
| | 2002] | | | |
| AAB19986 | Human camello 3 (Hcml13) | 42 . . . 195 | 144/154 (93%) | 1e−75 |
| | protein (partial) - Homo | 1 . . . 144 | 144/154 (93%) | |
| | sapiens, 144 aa. | | | |
| | [WO200077024-A1, 21 | | | |
| | DEC. 2000] | | | |
| AAB19985 | Human camello 2 (Hcml2) | 47 . . . 200 | 63/158 (39%) | 2e−21 |
| | protein - Homo | 56 . . . 203 | 92/158 (57%) | |
| | sapiens, 227 aa. | | | |
| | [WO200077024-A1, 21 DEC. | | | |
| | 2000] | | | |
| AAB19984 | Human camello 1 (Hcml1) | 41 . . . 196 | 60/160 (37%) | 1e−19 |
| | protein - Homo | 50 . . . 199 | 88/160 (54%) | |
| | sapiens, 227 aa. | | | |
| | [WO200077024-A1, 21 DEC. | | | |
| | 2000] | | | |

In a BLAST search of public sequence databases, the NOV16a protein was found to have homology to the proteins shown in the BLASTP data in Table 16D.

TABLE 16D
Public BLASTP Results for NOV16a

| | | NOV16a | Identities/ | |
|---|---|---|---|---|
| Protein | | Residues/ | Similarities | |
| Accession | Protein/Organism/ | Match | for the | Expect |
| Number | Length | Residues | Matched Portion | Value |
| Q8N9F0 | Hypothetical protein | 85 . . . 228 | 134/144 (93%) | 1e−69 |
| | FLJ37478 - Homo | 1 . . . 134 | 134/144 (93%) | |
| | sapiens (Human), 134 aa. | | | |
| Q8K065 | Hypothetical protein - Mus | 176 . . . 228 | 53/53 (100%) | 2e−23 |
| | musculus (Mouse), | 1 . . . 53 | 53/53 (100%) | |
| | 53 aa (fragment). | | | |
| Q9UHF3 | Putative N-acetyltransferase | 47 . . . 200 | 63/158 (39%) | 7e−21 |
| | Camello 2 - Homo | 56 . . . 203 | 92/158 (57%) | |
| | sapiens | | | |
| | (Human), 227 aa. | | | |
| Q9UQ17 | GLA protein - Homo | 41 . . . 196 | 60/160 (37%) | 4e−19 |
| | sapiens | 50 . . . 199 | 88/160 (54%) | |
| | (Human), 227 aa. | | | |
| Q9UHE5 | Putative N-acetyltransferase | 41 . . . 196 | 60/160 (37%) | 4e−19 |
| | CML1 - Homo | 50 . . . 199 | 88/160 (54%) | |
| | sapiens | | | |
| | (Human), 227 aa. | | | |

PFam analysis predicts that the NOV16a protein contains the domains shown in the Table 16E.

TABLE 16E
Domain Analysis of NOV16a

| | | Identities/ | |
|---|---|---|---|
| | | Similarities | |
| | NOV16a Match | for the | |
| Pfam Domain | Region | Matched Region | Expect Value |
| Acetyltransf | 111 . . . 191 | 28/82 (34%) | 1.8e−17 |
| | | 65/82 (79%) | |

### Example 17

The NOV17 clone was analyzed, and the nucleotide and encoded polypeptide sequences are shown in Table 17A.

TABLE 17A
NOV17 Sequence Analysis

| | | |
|---|---|---|
| NOV17a, CG57570-01 | SEQ ID NO: 65 | 1748 bp |
| DNA Sequence | ORF Start: ATG at 98 | ORF Stop: end of sequence |

GTTCACCCCAAGACTAAGTTCTTTCCCAAGTTAGAGAAGAAGAGAGAAAGCAAAAAGAAGAGAGGAAA
GTTCTCCCTTCCCCTCCTCCGTGCCTGTCATGTCCTCTAAGCCAGAGCCGAAGGACGTCCACCAACTG
AACGGGACTGGCCCTTCTGCCTCTCCCTGCTCTTCAGATGGCCCAGGGAGAGAGCCCTTGGCTGGGAC
CTCAGAGTTCCTGGGGCCTGATGGGGCTGGGGTAGAGGTGGTGATTGAGTCTCGGGCCAACGCCAAGG
GGGTTCGGGAGGAGGACGCCCTGCTGGAGAACGGGAGCCAGAGCAACGAAAGTGACGACGTCAGCACA
GACCGTGGCCCTGCGCCACCTTCCCCGCTCAAGGAGACCTCCTTTTCCATCGGGCTGCAAGTACTGTT
TCCATTCCTCCTGGCAGGCTTTGGGACCGTGGCTGCTGGCATGGTGTTGGACATCGTGCAGCACTGGG
AAGTCTTCCAGAAGGTGACAGAGGTCTTCATCCTAGTGCCTGCGCTGCTGGGGCTCAAAGGGAACCTG
GAAATGACCCTGGCATCAAGGCTTTCCACTGCAGCGAGTATCAACATTGGACACATGGACACACCCAA
GGAGCTCTGGCGGATGATCACTGGGAACATGGCCCTCATCCAGGTGCAGGCCACGGTGGTGGGCTTCC
TGGCGTCCATCGCAGCCGTCGTCTTTGGCTGGATCCCTGATGGCCACTTCAGTATTCCGCACGCCTTC
CTGCTCTGTGCTAGCAGCGTGGCCACAGCCTTCATTGCCTCCCTGGTACTGGGTATGATCATGATTGG
AGTCATCATTGGCTCTCGCAAGATTGGGATCAACCCAGACAATGTGGCCACACCCATTGCTGCCAGCC
TGGGCGACCTCATCACCTTGGCGCTGCTCTCAGGCATCAGCTGGGGACTCCTGACCTCTGCCCTCTCA
GATCACTGGCGATACATCTACCCACTGGTGTGTGCTTTCTTTGTGGCCCTGCTGCCTGTCTGGGTGGT
GCTGGCCCGACGAAGTCCAGCCACAAGGGAGGTGTTGTACTCGGGCTGGGAGCCTGTTATCATTGCCA
TGGCCATCAGCAGTGTGGGAGGCCTCATCTTGGACAAGACTGTCTCAGACCCCAACTTTGCTCGGATG
GCTGTCTTCACGCCTGTGATTAATGGTGTTGGGGGCAATCTGGTGGCAGTGCAGGCCAGCCGCATCTC
CACCTTCCTGCACATGAATGGAATGCCCGGAGAGAACTCTGAGCAAGCTCCTCGCCGCTGTCCCAGTC
CTTGTACCACCTTCTTCAGCCCTGGTGTGAATTCTCGCTCAGCCCGGGTCCTCTTCCTCCTCGTGGTC
CCAGGACACCTGGTGTTCCTCTACACCATCAGCTGTATGCAGGGCGGGCACACCACCCTCACACTCAT
CTTCATCATCTTCTATATGACAGCTGCACTGCTCCAGGTGCTGATTCTCCTGTACATCGCAGACTGGA
TGGTGCACTGGATGTGGGGCCGGGGCCTGGACCCGGACAACTTCTCCATCCCATACTTGACTGCTCTG
GGGGACCTGCTTGGCACTGGGCTCCTAGCACTCAGCTTCCATGTTCTCTGGCTCATAGGGGACCGAGA
CACGGATGTCGGGGACTAGCTTGGTCACTCAACATTTTCCCCATCCCTCTGCACTTTCTATTTGAAAT
TTTTCTTTTGTTCCCCTGTCCCTCCTCCACCCCACACTCCCACCTCTT

| | | | |
|---|---|---|---|
| NOV17a, CG57570-01 | SEQ ID NO: 66 | 517 aa | MW at 55097.3 kD |
| Protein Sequence | | | |

MSSKPEPKDVHQLNGTGPSASPCSSDGPGREPLAGTSEFLGPDGAGVEVVIESRANAKGVREEDALLE
NGSQSNESDDVSTDRGPAPPSPLKETSFSIGLQVLFPFLLAGFGTVAAGMVLDIVQHWEVFQKVTEVF
ILVPALLGLKGNLEMTLASRLSTAASINIGHMDTPKELWRMITGNMALIQVQATVVGFLASIAAVVFG
WIPDGHFSIPHAFLLCASSVATAFIASLVLGMIMIGVIIGSRKIGINPDNVATPIAASLGDLITLALL
SGISWGLLTSALSDHWRYIYPLVCAFFVALLPVWVVLARRSPATREVLYSGWEPVIIAMAISSVGGLI
LDKTVSDPNFAGMAVFTPVINGVGGNLVAVQASRISTFLHMNGMPGENSEQAPRRCPSPCTTFFSPGV
NSRSARVLFLLVVPGHLVFLYTISCMQGGHTTLTLIFIIFYMTAALLQVLILLYIADWMVHWMWGRGL
DPDNFSIPYLTALGDLLGTGLLALSFHVLWLIGDRDTDVCD

Further analysis of the NOV17a protein yielded the following properties shown in Table 17B.

TABLE 17B
Protein Sequence Properties NOV17a

| SignalP analysis: | No Known Signal Sequence Predicted | | | | |
|---|---|---|---|---|---|
| PSORT II analysis: | | | | | |
| | PSG: a new signal peptide prediction method | | | | |
| | N-region: length 9; pos. chg 2; neg. chg 2 | | | | |
| | H-region: length 16; peak value 3.46 | | | | |
| | PSG score: −0.94 | | | | |
| | GvH: von Heijne's method for signal seq. recognition | | | | |
| | GvH score (threshold: −2.1): −8.12 | | | | |
| | possible cleavage site: between 25 and 26 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 10 | | | | |
| | INTEGRAL | Likelihood = | −3.35 | Transmembrane | 107-123 |
| | INTEGRAL | Likelihood = | 0.21 | Transmembrane | 128-144 |
| | INTEGRAL | Likelihood = | −5.57 | Transmembrane | 190-206 |
| | INTEGRAL | Likelihood = | −10.72 | Transmembrane | 227-243 |
| | INTEGRAL | Likelihood = | −2.34 | Transmembrane | 259-275 |
| | INTEGRAL | Likelihood = | −10.77 | Transmembrane | 294-310 |
| | INTEGRAL | Likelihood = | −2.71 | Transmembrane | 326-342 |
| | INTEGRAL | Likelihood = | −6.26 | Transmembrane | 415-431 |
| | INTEGRAL | Likelihood = | −8.55 | Transmembrane | 443-459 |
| | INTEGRAL | Likelihood = | −3.50 | Transmembrane | 492-508 |

| PERIPHERAL | Likelihood = | 0.90 | (at 354) |
|---|---|---|---|
| ALOM score: −10.77 (number of TMSs: 10) | | | |
| MTOP: Prediction of membrane topology (Hartmann et al.) | | | |
| Center position for calculation: 114 | | | |
| Charge difference: −1.5 C(−1.5)-N(0.0) | | | |
| N >= C: N-terminal side will be inside | | | |
| >>> membrane topology: type 3a | | | |
| MITDISC: discrimination of mitochondrial targeting seq | | | |
| R content: | 0 | Hyd Moment (75): | 5.28 |
| Hyd Moment (95): | 4.64 | G content: | 0 |
| D/E content: | 2 | S/T content: | 2 |

| Score: −6.73 | |
|---|---|
| Gavel: prediction of cleavage sites for mitochondrial preseq | |
| cleavage site motif not found | |
| NUCDISC: discrimination of nuclear localization signals | |
| pat4: none | |
| pat7: none | |
| bipartite: none | |
| content of basic residues: 5.2% | |
| NLS Score: −0.47 | |
| KDEL: ER retention motif in the C-terminus: none | |
| ER Membrane Retention Signals: none | |
| SKL: peroxisomal targeting signal in the C-terminus: none | |
| PTS2: 2nd peroxisomal targeting signal: none | |
| VAC: possible vacuolar targeting motif: none | |
| RNA-binding motif: none | |
| Actinin-type actin-binding motif: | |
| type 1: none | |
| type 2: none | |
| NMYR: N-myristoylation pattern: none | |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: | |
| Leucine zipper pattern (PS00029): *** found *** | |
| LTALGDLLGTGLLALSFHVLWL at 486 | |
| none | |
| checking 71 PROSITE ribosomal protein motifs: none | |
| checking 33 PROSITE prokaryotic DNA binding motifs: none | |
| NNCN: Reinhardt's method for Cytoplasmic/Nuclear discrimination | |
| Prediction: cytoplasmic | |
| Reliability: 94.1 | |
| COIL: Lupas's algorithm to detect coiled-coil regions | |
| total: 0 residues | |
| Final Results (k = 9/23): | |
| 66.7%: | endoplasmic reticulum |
| 22.2%: | mitochondrial |
| 11.1%: | vesicles of secretory system |

>> prediction for CG57570-01 is end (k=9)

A search of the NOV17a protein against the Geneseq database, a proprietary database that contains sequences published in patents and patent publication, yielded several homologous proteins shown in Table 17C.

TABLE 17C
Geneseq Results for NOV17a

| | | NOV17a | Identities/ | |
|---|---|---|---|---|
| | Protein/Organism/ | Residues/ | Similarities | |
| Geneseq | Length | Match | for the | Expect |
| Identifier | [Patent #, Date] | Residues | Matched Region | Value |
| ABB08638 | Human transporter protein SEQ | 1 . . . 517 | 506/517 (97%) | 0.0 |
| | ID NO 2 - Homo | 1 . . . 513 | 507/517 (97%) | |
| | sapiens, 513 aa. | | | |
| | [WO200190360-A2, 29 NOV. | | | |
| | 2001] | | | |
| ABP74113 | Human TRICH SEQ ID NO 18 - | 62 . . . 517 | 307/457 (67%) | e−173 |
| | Homo | 125 . . . 573 | 373/457 (81%) | |
| | sapiens, 573 aa. | | | |
| | [WO200246415-A2, 13 JUN. | | | |
| | 2002] | | | |
| AAB95482 | Human protein sequence SEQ ID | 62 . . . 517 | 307/457 (67%) | e−173 |
| | NO: 18007 - Homo | 42 . . . 490 | 373/457 (81%) | |
| | sapiens, 490 | | | |
| | aa. [EP1074617-A2, 07 FEB. | | | |
| | 2001] | | | |
| AAY94882 | Human protein clone | 60 . . . 508 | 246/451 (54%) | e−129 |
| | HP10031 -Homo | 33 . . . 470 | 315/451 (69%) | |
| | sapiens, 487 aa. | | | |
| | [WO200005367-A2, | | | |
| | 03 FEB. 2000] | | | |
| AAM47910 | Human initiation | 107 . . . 508 | 227/404 (56%) | e−121 |
| | factor 46 -Homo | 1 . . . 397 | 290/404 (71%) | |
| | sapiens, 414 aa. | | | |
| | [CN1307045-A, 08 AUG. 2001] | | | |

In a BLAST search of public sequence databases, the NOV17a protein was found to have homology to the proteins shown in the BLASTP data in Table 17D.

TABLE 17D
Public BLASTP Results for NOV17a

| | | NOV17a | Identities/ | |
|---|---|---|---|---|
| Protein | | Residues/ | Similarities | |
| Accession | Protein/Organism/ | Match | for the | Expect |
| Number | Length | Residues | Matched Portion | Value |
| Q8IVJ1 | Solute carrier family 41 member | 1 . . . 517 | 502/517 (97%) | 0.0 |
| | 1 - Homo | 1 . . . 513 | 504/517 (97%) | |
| | sapiens (Human), 513 | | | |
| | aa. | | | |
| Q8BJA2 | Hypothetical divalent cation | 1 . . . 517 | 496/517 (95%) | 0.0 |
| | transporter containing protein- | 1 . . . 512 | 499/517 (95%) | |
| | Mus musculus (Mouse), 512 aa. | | | |
| Q8BYR8 | Hypothetical divalent cation | 62 . . . 517 | 306/456 (67%) | e−175 |
| | transporter containing protein- | 126 . . . 573 | 373/456 (81%) | |
| | Mus musculus (Mouse), 573 aa. | | | |
| Q9H0E5 | Hypothetical protein | 62 . . . 517 | 307/457 (67%) | e−173 |
| | DKFZp434K0427 - Homo | 42 . . . 490 | 373/457 (81%) | |
| | sapiens (Human), 490 aa. | | | |
| Q96JW4 | Hypothetical protein FLJ14932- | 62 . . . 517 | 307/457 (67%) | e−173 |
| | Homo | 42 . . . 490 | 373/457 (81%) | |
| | sapiens (Human), 490 aa. | | | |

PFam analysis predicts that the NOV17a protein contains the domains shown in the Table 17E.

TABLE 17E
Domain Analysis of NOV17a

| | | Identities/ | |
|---|---|---|---|
| | | Similarities | |
| Pfam | NOV17a | for the | |
| Domain | Match Region | Matched Region | Expect Value |
| MgtE | 138 . . . 276 | 39/140 (28%) | 2.8e−26 |
| | | 117/140 (84%) | |
| MgtE | 356 . . . 503 | 33/153 (22%) | 1.1e−06 |
| | | 103/153 (67%) | |

### Example 18

The NOV18 clone was analyzed, and the nucleotide and encoded polypeptide sequences are shown in Table 18A.

TABLE 18A
NOV18 Sequence Analysis

| | | |
|---|---|---|
| NOV18a, CG57758-02 | SEQ ID NO: 67 | 1899 bp |
| DNA Sequence | ORF Start: ATG at 31 | ORF Stop: end of sequence |

CGTCTCGCCCGCCAGTCTCCCTCCCGCGCGATGGCCTCGGCGCTGAGCTATGTCTCCAAGTTCAAGTC
CTTCGTGATCTTGTTCGTCACCCCGCTCCTGCTGCTGCCACTCGTCATTCTGATGCCCGCCAAGGTCA
GTTGCTGTGCCTACGTCATCATCCTCATGGCCATTTACTGGTGCACAGAAGTCATCCCTCTGGCTGTC
ACCTCTCTCATGCCTGTCTTGCTTTTCCCACTCTTCCAGATTCTGGACTCCAGGCAGGTGTGTGTCCA
GTACATGAAGGACACCAACATGCTGTTCCTGGGCGGCCTCATCGTGGCCGTGGCTGTGGAGCGCTGGA
ACCTGCACAAGAGGATCGCCCTGCGCACGCTCCTCTGGGTGGGGGCCAAGCCTGCACGGCTGATGCTG
GGCTTCATGGGCGTCACAGCCCTCCTGTCCATGTGGATCAGTAACACGGCAACCACGGCCATGATGGT
GCCCATCGTGGAGGCCATATTGCAGCAGATGGAAGCCACAAGCGCAGCCACCGAGGCCGGCCTGGAGG
GACAAGGTACCACAATAAACAACCTGAATGCACTGGAGGATGATACAGTGAAAGCAGTACTAGGAGGA
AAGTGTGTAGCTATAATAAGCACTTACGTCAAAAAAGTAGAAAAACTTCAAATAAACAATCTAATGAC
ACCTCTTAAAAAACTAGAAAAGCAAGAGCAACAGGACCTAGGGCCTGGCATCAGGCCTCAGGACTCTG
CCCAGTGCCAGGAAGACCAAGAGCGGAAGAGGTTGTGTAAGGCCATGACCCTGTGCATCTGCTACGCG
GCCAGCATCGGGGGCACCGCCACCCTGACCGGGACGGGACCCAACGTGGTGCTCCTGGGCCAGATGAA
CGAGTTGTTTCCTGACAGCAAGGACCTCGTGAACTTTGCTTCCTGGTTTGCATTTGCCTTTCCCAACA
TGCTGGTGATGCTGCTGTTCGCCTGGCTGTGGCTCCAGTTTGTTTACATGTTCTCCAGTTTTAAAAAG
TCCTGGGGCTGCGGGCTAGAGAGCAAGAAAAACGAGAAGGCTGCCCTCAAGGTGCTGCAGGAGGAGTA
CCGGAAGCTGGGGCCCTTGTCCTTCGCGGAGATCAACGTGCTGATCTGCTTCTTCCTGCTGGTCATCC
TGTGGTTCTCCCGAGACCCCGGCTTCATGCCCGGCTGGCTGACTGTTGCCTGGGTGGAGGGTGAGACA
AAGTCAGTCTCCGATGCCACTGTGGCCATCTTTGTGGCCACCCTGCTATTCATTGTGCCTTCACAGAA
GCCCAAGTTTAACTTCCGCAGCCAGACTGAGGAAGGTAAGTCTCCTGTTCTGATCGCCCCCCCTCCCC
TGCTGGATTGGAAGGTAACCCAGGAGAAAGTGCCCTGGGGCATCGTGCTGCTACTAGGGGGCGGATTT
GCTCTGGCTAAAGGATCCGAGGCCTCGGGGCTGTCCGTGTGGATGGGCAAGCAGATGGAGCCCTTGCA
CGCAGTGCCCCCGGCAGCCATCACCTTGATCTTGTCCTTGCTCGTTGCCGTGTTCACTGAGTGCACAA
GCAACGTGGCCACCACCACCTTGTTCCTGCCCATCTTTGCCTCCATGTCTCGCTCCATCGGCCTCAAT
CCGCTGTACATCATGCTGCCCTGTACCCTGAGTGCCTCCTTTGCCTTCATGTTGCCTGTGGCCACCCC
TCCAAATGCCATCGTGTTCACCTATGGGCACCTCAAGGTTGCTGACATGGTAAAAACAGGAGTCATAA
TGAACATAATTGGAGTCTTCTGTGTGTTTTTGGCTGTCAACACCTGGGGACGGGCCATATTTGACTTG
GATCATTTCCCTGACTGGGCTAATGTGACACATATTGAGACTTAGGAAGAGCCACAAGACCAC

| | | | |
|---|---|---|---|
| NOV18a, CG57758-02 | SEQ ID NO: 68 | 616 aa | MW at 67816.9 kD |
| Protein Sequence | | | |

MASALSYVSKFKSFVILFVTPLLLLPLVILMPAKVSCCAYVIILMAIYWCTEVIPLAVTSLMPVLLFP
LFQILDSRQVCVQYMKDTNMLFLGGLIVAVAVERWNLHKRIALRTLLWVGAKPARLMLGFMGVTALLS
MWISNTATTAMMVPIVEAILQQMEATSAATEAGLEGQGTTINNLNALEDDTVKAVLGGKCVAIISTYV
KKVEKLQINNLMTPLKKLEKQEQQDLGPGIRPQDSAQCQEDQERKRLCKAMTLCICYAASIGGTATLT
GTGPNVVLLGQMNELFPDSKDLVNFASWFAFAFPNMLVMLLFAWLWLQFVYMFSSFKKSWGCGLESKK
NEKAALKVLQEEYRKLGPLSFAEINVLICFFLLVILWFSRDPGFMPGWLTVAWVEGETKSVSDATVAI
FVATLLFIVPSQKPKFNFRSQTEEGKSPVLIAPPPLLDWKVTQEKVPWGIVLLLGGGFALAKGSEASG
LSVWMGKQMEPLHAVPPAAITLILSLLVAVFTECTSNVATTTLFLPIFASMSRSIGLNPLYIMLPCTL
SASFAFMLPVATPPNAIVFTYGHLKVADMVKTGVIMNIIGVFCVFLAVNTWGRAIFDLDHFPDWANVT
HIET

| | | |
|---|---|---|
| NOV18b, CG57758-01 | SEQ ID NO: 69 | 1790 bp |
| DNA Sequence | ORF Start: ATG at 16 | ORF Stop: end of sequence |

TCTCCCTCCCGCGCGATGGCCTCGGCGCTGAGCTATGTCTCCAAGTTCAAGTCCTTCGTGATCTTGTT
CGTCACCCCGCTCCTGCTGCTGCCACTCGTCATTCTGATGCCCGCCAAGGTCAGTTGTGCCTACGTCA
TCATCCTCATGGCCATTTACTGGTGCACAGAAGTCATCCCTCTGGCTGTCACCTCTCTCATGCCTGTC
TTGCTTTTCCCACTCTTCCAGATTCTGGACTCCAGGCAGGTGTGTGTCCAGTACATGAAGGACACCAA
CATGCTGTTCCTGGGCGGCCTCATCGTGGCCGTGGCTGTGGAGCGCTGGAACCTGCACAAGAGGATCG
CCCTGCGCACGCTCCTCTGGGTGGGGGCCAAGCCTGCACGGCTGATGCTGGGCTTCATGGGCGTCACA
GCCCTCCTGTCCATGTGGATCAGTAACACGGCAACCACGGCCATGATGGTGCCCATCGTGGAGGCCAT
ATTGCAGCAGATGGAAGCCACAAGCGCAGCCACCGAGGCCGGCCTGGAGCTGGTGGACAAGGGCAAGG
CCAAGGAGCTGCCAGGGAGTCAAGTGATTTTTGAAGGCCCCACTCTGGGGCAGCAGGAAGACCAAGAG
CGGAAGAGGTTGTGTAAGGCCATGACCCTGTGCATCTGCTACGCGGCCAGCATCGGGGGCACCGCCAC
CCTGACCGGGACGGGACCCAACGTGGTGCTCCTGGGCCAGATGAACGAGTTGTTTCCTGACAGCAAGG
ACCTCGTGAACTTTGCTTCCTGGTTTGCATTTGCCTTTCCCAACATGCTGGTGATGCTGCTGTTCGCC
TGGCTGTGGCTCCAGTTTGTTTACATGTTCTCCAGTTTTAAAAAGTCCTGGGGCTGCGGGCTAGAGAG
CAAGAAAAACGAGAAGGCTGCCCTCAAGGTGCTGCAGGAGGAGTACCGGAAGCTGGGGCCCTTGTCCT
TCGCGGAGATCAACGTGCTGATCTGCTTCTTCCTGCTGGTCATCCTGTGGTTCTCCCGAGACCCCGGC
TTCATGCCCGGCTGGCTGACTGTTGCCTGGGTGGAGGGTGAGACAAAGTATGTCTCCGATGCCACTGT
GGCCATCTTTGTGGCCACCCTGCTATTCATTGTGCCTTCACAGAAGCCCAAGTTTAACTTCCGCAGCC
AGACTGAGGAAGGTAAGTCTCCTGTTCTGATCGCCCCCCCTCCCCTGCTGGATTGGAAGGTAACCCAG
GAGAAAGTGCCCTGGGGCATCGTGCTGCTACTAGGGGGCGGATTTGCTCTGGCTAAAGGATCCGAGGC
CTCGGGGCTGTCCGTGTGGATGGGGAAGCAGATGGAGCCCTTGCACGCAGTGCCCCCGGCAGCCATCA
CCTTGATCTTGTCCTTGCTCGTTGCCGTGTTCACTGAGTGCACAAGCAACGTGGCCACCACCACCTTG
TTCCTGCCCATCTTTGCCTCCATGTCTCGCTCCATCGGCCTCAATCCGCTGTACATCATGCTGCCCTG
TACCCTGAGTGCCTCCTTTGCCTTCATGTTGCCTGTGGCCACCCCTCCAAATGCCATCGTGTTCACCT
ATGGGCACCTCAAGGTTGCTGACATGGTGAAAACAGGAGTCATAATGAACATAATTGGAGTCTTCTGT
GTGTTTTTGGCTGTCAACACCTGGGGACGGGCCATATTTGACTTGGATCATTTCCCTGACTGGGCTAA
TGTGACACATATTGAGACTTAGGAAGAGCCACAAGACCACACACACAGCCCTTACCCTCCTCAGGACT
ACCGAACCTTCTGGCACACCTT

| | | | |
|---|---|---|---|
| NOV18b, CG57758-01 | SEQ ID NO: 70 | 568 aa | MW at 62592.9 kD |
| Protein Sequence | | | |

MASALSYVSKFKSFVILFVTPLLLLPLVILMPAKVSCAYVIILMAIYWCTEVIPLAVTSLMPVLLFPL
FQILDSRQVCVQYMKDTNMLFLGGLIVAVAVERWNLHKRIALRTLLWVGAKPARLMLGFMGVTALLSM
WISNTATTAMMVPIVEAILQQMEATSAATEAGLELVDKGKAKELPGSQVIFEGPTLGQQEDQERKRLC
KAMTLCICYAASIGGTATLTGTGPNVVLLGQMNELFPDSKDLVNFASWFAFAFPNMLVMLLFAWLWLQ
FVYMFSSFKKSWGCGLESKKNEKAALKVLQEEYRKLGPLSFAEINVLICFFLLVILWFSRDPGFMPGW
LTVAWVEGETKYVSDATVAIFVATLLFIVPSQKPKFNFRSQTEEGKSPVLIAPPPLLDWKVTQEKVPW
GIVLLLGGGFALAKGSEASGLSVWMGKQMEPLHAVPPAAITLILSLLVAVFTECTSNVATTTLFLPIF
ASMSRSIGLNPLYIMLPCTLSASFAFMLPVATPPNAIVFTYGHLKVADMVKTGVIMNIIGVFCVFLAV
NTWGRAIFDLDHFPDWANVTHIET

| | | |
|---|---|---|
| NOV18c, CG57758-03 | SEQ ID NO: 71 | 3147 bp |
| DNA Sequence | ORF Start: ATG at 2 | ORF Stop: end of sequence |

GATGGCCTCGGCGCTGAGCTATGTCTCCAAGTTCAAGTCCTTCGTGATCTTGTTCGTCACCCCGCTCC
TGCTGCTGCCACTCGTCATTCTGATGCCCGCCAAGTTTGTCAGGTGTGCCTACGTCATCATCCTCATG
GCCATTTACTGGTGCACAGAAGTCATCCCTCTGGCTGTCACCTCTCTCATGCCTGTCTTGCTTTTCCC
ACTCTTCCAGATTCTGGACTCCAGGCAGGTGTGTGTCCAGTACATGAAGGACACCAACATGCTGTTCC
TGGGCGGCCTCATCGTGGCCGTGGCTGTGGAGCGCTGGAACCTGCACAAGAGGATCGCCCTGCGCACG
CTCCTCTGGGTGGGGGCCAAGCCTGCACGGCTGATGCTGGGCTTCATGGGCGTCACAGCCCTCCTGTC
CATGTGGATCAGTAACACGGCAACCACGGCCATGATGGTGCCCATCGTGGAGGCCATATTGCAGCAGA
TGGAAGCCACAAGCGCAGCCACCGAGGCCGGCCTGGAGCTGGTGGACAAGGGCAAGGCCAAGGAGCTG
CCAGGGAGTCAAGTGATTTTTGAAGGCCCCACTCTGGGGCAGCAGGAAGACCAAGAGCGGAAGAGGTT
GTGTAAGGCCATGACCCTGTGCATCTGCTACGCGGCCAGCATCGGGGGCACCGCCACCCTGACCGGGA
CGGGACCCAACGTGGTGCTCCTGGGCCAGATGAACGAGTTGTTTCCTGACAGCAAGGACCTCGTGAAC
TTTGCTTCCTGGTTTGCATTTGCCTTTCCCAACATGCTGGTGATGCTGCTGTTCGCCTGGCTGTGGCT
CCAGTTTGTTTACATGAGATTCAATTTTAAAAAGTCCTGGGGCTGCGGGCTAGAGAGCAAGAAAAACG
AGAAGGCTGCCCTCAAGGTGCTGCAGGAGGAGTACCGGAAGTTGGGGCCCTTGTCCTTCGCGGAGATC
AACGTGCTGATCTGCTTCTTCCTGCTGGTCATCCTGTGGTTCTCCCGAGACCCCGGCTTCATGCCCGG
CTGGCTGACTGTTGCCTGGGTGGAGGGTGAGACAAAGTATGTCTCCGATGCCACTGTGGCCATCTTTG
TGGCCACCCTGCTATTCATTGTGCCTTCACAGAAGCCCAAGTTTAACTTCCGCAGCCAGACTGAGGAA
GAAAGGAAAACTCCATTTTATCCCCCTCCCCTGCTGGATTGGAAGGTAACCCAGGAGAAAGTGCCCTG
GGGCATCGTGCTGCTACTAGGGGGCGGATTTGCTCTGGCTAAAGGATCCGAGGCCTCGGGGCTGTCCG
TGTGGATGGGGAAGCAGATGGAGCCCTTGCACGCAGTGCCCCCGGCAGCCATCACCTTGATCTTGTCC
TTGCTCGTTGCCGTGTTCACTGAGTGCACAAGCAACGTGGCCACCACCACCTTGTTCCTGCCCATCTT
TGCCTCCATGTCTCGCTCCATCGGCCTCAATCCGCTGTACATCATGCTGCCCTGTACCCTGAGTGCCT
CCTTTGCCTTCATGTTGCCTGTGGCCACCCCTCCAAATGCCATCGTGTTCACCTATGGGCACCTCAAG
GTTGCTGACATGGTGAAAACAGGAGTCATAATGAACATAATTGGAGTCTTCTGTGTGTTTTTGGCTGT
CAACACCTGGGGACGGGCCATATTTGACTTGGATCATTTCCCTGACTGGGCTAATGTGACACATATTG
AGACTTAGGAAGAGCCACAAGACCACACACACAGCCCTTACCCTCCTCAGGACTACCGAACCTTCTGG
CACACCTTGTACAGAGTTTTGGGGTTCACACCCCAAAATGACCCAACGATGTCCACACACCACCAAAA
CCCAGCCAATGGGCCACCTCTTCCTCCAAGCCCAGATGCAGAGATGGTCATGGGCAGCTGGAGGGTAG
GCTCAGAAATGAAGGGAACCCCTCAGTGGGCTGCTGGACCCATCTTTCCCAAGCCTTGCCATTATCTC
TGTGAGGGAGGCCAGGTAGCCGAGGGATCAGGATGCAGGCTGCTGTACCCGCTCTGCCTCAAGCATCC
CCCACACAGGGCTCTGGTTTTCACTCGCTTCGTCCTAGATAGTTTAAATGGGAATCGGATCCCCTGGT
TGAGAGCTAAGACAACCACCTACCAGTGCCCATGTCCCTTCCAGCTCACCTTGAGCAGCCTCAGATCA
TCTCTGTCACTCTGGAAGGGACACCCCAGCCAGGGACGGAATGCCTGGTCTTGAGCAACCTCCCACTG
CTGGAGTGCGAGTGGGAATCAGAGCCTCCTGAAGCCTCTGGGAACTCCTCCTGTGGCCACCACCAAAG
GATGAGGAATCTGAGTTGCCAACTTCAGGACGACACCTGGCTTGCCACCCACAGTGCACCACAGGCCA
ACCTACGCCCTTCATCACTTGGTTCTGTTTTAATCGACTGGCCCCCTGTCCCACCTCTCCAGTGAGCC
TCCTTCAACTCCTTGGTCCCCTGTTGTCTGGGTCAACATTTGCCGAGACGCCTTGGCTGGCACCCTCT
GGGGTCCCCCTTTTCTCCCAGGCAGGTCATCTTTTCTGGCAGATGCTTCCCCTGCCATCCCCAAATAG
CTAGGATCACACTCCAAGTATGGGCAGTGATGGCGCTCTGGGGGCCACAGTGGGCTATCTAGGCCCTC
CCTCACCTGAGGCCCAGAGTGGACACAGCTGTTAATTTCCACTGGCTATGCCACTTCAGAGTCTTTCA
TGCCAGCGTTTGAGCTCCTCTGGGTAAAATCTTCCCTTTGTTGACTGGCCTTCACAGCCATGGCTGGT
GACAACAGAGGATCGTTGAGATTGAGCAGCGCTTGGTGATCTCTCAGCAAACAACCCCTCCCCGTGGG
CCAATCTACTTGAAGTTACTCGGACAAAGACCCCAAAGTGGGGCAACAACTCCAGAGAGGCTGTGGGA
ATCTTCAGAAGCCCCCCTGTAAGAGACAGACATGAGAGACAAGCATCTTCTTTCCCCCGCAAGTCCAT
TTTATTTCCTTCTTGTGCTGCTCTGGAAGAGAGGCAGTAGCAAAGAGATGAGCTCCTGGATGGCATTT
TCCAGGGCAGGAGAAAGTATGAGAGCCTCAGGAAACCCCATCAAGGACCGAGTATGTGTCTGGTTCCT
TTGGTGGTTGGCTTCTGGC

| | | | |
|---|---|---|---|
| NOV18c, CG57758-03 | SEQ ID NO: 72 | 568 aa | MW at 63061.4 kD |
| Protein Sequence | | | |

MASALSYVSKFKSFVILFVTPLLLLPLVILMPAKFVRCAYVIILMAIYWCTEVIPLAVTSLMPVLLFP
LFQILDSRQVCVQYMKDTNMLFLGGLIVAVAVERWNLHKRIALRTLLWVGAKPARLMLGFMGVTALLS
MWISNTATTAMMVPIVEAILQQMEATSAATEAGLELVDKGKAKELPGSQVIFEGPTLGQQEDQERKRL
CKAMTLCICYAASIGGTATLTGTGPNVVLLGQMNELFPDSKDLVNFASWFAFAFPNMLVMLLFAWLWL
QFVYMRFNFKKSWGCGLESKKNEKAALKVLQEEYRKLGPLSFAEINVLICFFLLVILWFSRDPGFMPG
WLTVAWVEGETKYVSDATVAIFVATLLFIVPSQKPKFNFRSQTEEERKTPFYPPPLLDWKVTQEKVPW
GIVLLLGGGFALAKGSEASGLSVWMGKQMEPLHAVPPAAITLILSLLVAVFTECTSNVATTTLFLPIF
ASMSRSIGLNPLYIMLPCTLSASFAFMLPVATPPNAIVFTYGHLKVADMVKTGVIMNIIGVFCVFLAV
NTWGRAIFDLDHFPDWANVTHIET

| | | |
|---|---|---|
| NOV18d, CG57758-04 | SEQ ID NO: 73 | 1606 bp |
| DNA Sequence | ORF Start: ATG at 2 | ORF Stop: end of sequence |

GATGGCCTCGGCGCTGAGCTATGTCTCCAAGTTCAAGTCCTTCGTGATCTTGTTCGTCACCCCGCTCC
TGCTGCTGCCACTCGTCATTCTGATGCCCGCCAAGTTTGTCAGGTGTGCCTACGTCATCATCCTCATG
GCCATTTACTGGTGCACAGAAGTCATCCCTCTGGCTGTCACCTCTCTCATGCCTGTCTTGCTTTTCCC
ACTCTTCCAGATTCTGGACTCCAGGCAGGTGTGTGTCCAGTACATGAAGGACACCAACATGCTGTTCC
TGGGCGGCCTCATCGTGGCCGTGGCTGTGGAGCGCTGGAACCTGCACAAGAGGATCGCCCTGCGCACG
CTCCTCTGGGTGGGGGCCAAGCCTGCACGGCTGATGCTGGGCTTCATGGGCGTCACAGCCCTCCTGTC
CATGTGGATCAGTAACACGGCAACCACGGCCATGATGGTGCCCATCGTGGAGGCCATATTGCAGCAGA
TGGAAGCCACAAGCGCAGCCACCGAGGCCGGCCTGGAGCTGGTGGACAAGGGCAAGGCCAAGGAGCTG
CCAGGGAGTCAAGTGATTTTTGAAGGCCCCACTCTGGGGCAGCAGGAAGACCAAGAGCGGAAGAGGTT
GTGTAAGGCCATGACCCTGTGCATCTGCTACGCGGCCAGCATCGGGGGCACCGCCACCCTGACCGGGA
CGGGACCCAACGTGGTGCTCCTGGGCCAGATGAACGAGTTGTTTCCTGACAGCAAGGACCTCGTGAAC
TTTGCTTCCTGGTTTGCATTTGCCTTTCCCAACATGCTGGTGATGCTGCTGTTCGCCTGGCTGTGGCT
CCAGTTTGTTTACATGAGATTCAATTTTAAAAAGTCCTGGGGCTGCGGGCTAGAGAGCAAGAAAAACG
AGAAGGCTGCCCTCAAGGTGCTGCAGGAGGAGTACCGGAAGTTGGGGCCCTTGTCCTTCGCGGAGATC
AACGTGCTGATCTGCTTCTTCCTGCTGGTCATCCTGTGGTTCTCCCGAGACCCCGGCTTCATGCCCGG
CTGGCTGACTGTTGCCTGGGTGGAGGGTGAGACAAAGTATGTCTCCGATGCCACTGTGGCCATCTTTG
TGGCCACCCTGCTATTCATTGTGCCTTCACAGAAGCCCAAGTTTAACTTCCGCAGCCAGACTGAGGAA
GAAAGGAAAACTCCATTTTATCCCCCTCCCCTGCTGGATTGGAAGGTAACCCAGGAGAAAGTGCCCTG
GGGCATCGTGCTGCTACTAGGGGGCGGATTTGCTCTGGCTAAAGGATCCGAGGCCTCGGGGCTGTCCG
TGTGGATGGGGAAGCAGATGGAGCCCTTGCACGCAGTGCCCCCGGCAGCCATCACCTTGATCTTGTCC
TTGCTCGTTGCCGTGTTCACTGAGTGCACAAGCAACGTGGCCACCACCACCTTGTTCCTGCCCATCTT
TGCCTCCATGGTGAAAACAGGAGTCATAATGAACATAATTGGAGTCTTCTGTGTGTTTTTGGCTGTCA
ACACCTGGGGACGGGCCATATTTGACTTGGATCATTTCCCTGACTGGGCTAATGTGACACATATTGAG
ACTTAGGAAGAGCCACAAGACCACACACATAGCCCTTACCCT

| | | | |
|---|---|---|---|
| NOV18d, CG57758-04 | SEQ ID NO: 74 | 522 aa | MW at 58109.6 kD |
| Protein Sequence | | | |

MASALSYVSKFKSFVILFVTPLLLLPLVILMPAKFVRCAYVIILMAIYWCTEVIPLAVTSLMPVLLFP
LFQILDSRQVCVQYMKDTNMLFLGGLIVAVAVERWNLHKRIALRTLLWVGAKPARLMLGFMGVTALLS
MWISNTATTAMMVPIVEAILQQMEATSAATEAGLELVDKGKAKELPGSQVIFEGPTLGQQEDQERKRL
CKAMTLCICYAASIGGTATLTGTGPNVVLLGQMNELFPDSKDLVNFASWFAFAFPNMLVMLLFAWLWL
QFVYMRFNFKKSWGCGLESKKNEKAALKVLQEEYRKLGPLSFAEINVLICFFLLVILWFSRDPGFMPG
WLTVAWVEGETKYVSDATVAIFVATLLFIVPSQKPKFNFRSQTEEERKTPFYPPPLLDWKVTQEKVPW
GIVLLLGGGFALAKGSEASGLSVWMGKQMEPLHAVPPAAITLILSLLVAVFTECTSNVATTTLFLPIF
ASMVKTGVIMNIIGVFCVFLAVNTWGRAIFDLDHFPDWANVTHIET

| | | |
|---|---|---|
| NOV18e, CG57758-05 | SEQ ID NO: 75 | 1781 bp |
| DNA Sequence | ORF Start: ATG at 2 | ORF Stop: end of sequence |

GATGGCCTCGGCGCTGAGCTATGTCTCCAAGTTCAAGTCCTTCGTGATCTTGTTCGTCACCCCGCTCC
TGCTGCTGCCACTCGTCATTCTGATGCCCGCCAAGTTTGTCAGGTGTGCCTACGTCATCATCCTCATG
GCCATTTACTGGTGCACAGAAGTCATCCCTCTGGCTGTCACCTCTCTCATGCCTGTCTTGCTTTTCCC
ACTCTTCCAGATTCTGGACTCCAGGCAGGTGTGTGTCCAGTACATGAAGGACACCAACATGCTGTTCC
TGGGCGGCCTCATCGTGGCCGTGGCTGTGGAGCGCTGGAACCTGCACAAGAGGATCGCCCTGCGCACG
CTCCTCTGGGTGGGGGCCAAGCCTGCACGGCTGATGCTGGGCTTCATGGGCGTCACAGCCCTCCTGTC
CATGTGGATCAGTAACACGGCAACCACGGCCATGATGGTGCCCATCGTGGAGGCCATATTGCAGCAGA
TGGAAGCCACAAGCGCAGCCACCGAGGCCGGCCTGGAGCTGGTGGACAAGGGCAAGGCCAAGGAGCTG
CCAGGGAGTCAAGTGATTTTTGAAGGCCCCACTCTGGGGCAGCAGGAAGACCAAGAGCGGAAGAGGTT
GTGTAAGGCCATGACCCTGTGCATCTGCTACGCGGCCAGCATCGGGGGCACCGCCACCCTGACCGGGA
CGGGACCCAACGTGGTGCTCCTGGGCCAGATGAACGAGTTGTTTCCTGACAGCAAGGACCTCGTGAAC
TTTGCTTCCTGGTTTGCATTTGCCTTTCCCAACATGCTGGTGATGCTGCTGTTCGCCTGGCTGTGGCT
CCAGTTTGTTTACATGAGATTCAATTTTAAAAAGTCCTGGGGCTGCGGGCTAGAGAGCAAGAAAAACG
AGAAGGCTGCCCTCAAGGTGCTGCAGGAGGAGTACCGGAAGTTGGGGCCCTTGTCCTTCGCGGAGATC
AACGTGCTGATCTGCTTCTTCCTGCTGGTCATCCTGTGGTTCTCCCGAGACCCCGGCTTCATGCCCGG
CTGGCTGACTGTTGCCTGGGTGGAGGGTGAGACAAAGTATGTCTCCGATGCCACTGTGGCCATCTTTG
TGGCCACCCTGCTATTCATTGTGCCTTCACAGAAGCCCAAGTTTAACTTCCGCAGCCAGACTGAGGAA
GAAAGGAAAACTCCATTTTATCCCCCTCCCCTGCTGGATTGGAAGGTAACCCAGGAGAAAGTGCCCTG
GGGCATCGTGCTGCTACTAGGGGGCGGATTTGCTCTGGCTAAAGGATCCGAGGCCTCGGGGCTGTCCG
TGTGGATGGGGAAGCAGATGGAGCCCTTGCACGCAGTGCCCCCGGCAGCCATCACCTTGATCTTGTCC
TTGCTCGTTGCCGTGTTCACTGAGTGCACAAGCAACGTGGCCACCACCACCTTGTTCCTGCCCATCTT
TGCCTCCATGAATCACGTCCCCAAGAGCTTCTGTGTTCTGTACGGTGATGTTGCAGTGCTGTCTTTCC
GCAGTCTCGCTCCATCGGCCTCAATCCGCTGTACATCATGCTGCCCTGTACCCTGAGTGCCTCCTTTG
CCTTCATGTTGCCTGTGGCCACCCCTCCAAATGCCATCGTGTTCACCTATGGGCACCTCAAGGTTGCT
GACATGGTGAAAACAGGAGTCATAATGAACATAATTGGAGTCTTCTGTGTGTTTTTGGCTGTCAACAC
CTGGGGACGGGCCATATTTGACTTGGATCATTTCCCTGACTGGGCTAATGTGACACATATTGAGACTT
AGGAAGAGCCACA

| | | | |
|---|---|---|---|
| NOV18e, CG57758-05 | SEQ ID NO: 76 | 516 aa | MW at 57173.5 kD |
| Protein Sequence | | | |

MASALSYVSKFKSFVILFVTPLLLLPLVILMPAKFVRCAYVIILMAIYWCTEVIPLAVTSLMPVLLFP
LFQILDSRQVCVQYMKDTNMLFLGGLIVAVAVERWNLHKRIALRTLLWVGAKPARLMLGFMGVTALLS
MWISNTATTAMMVPIVEAILQQMEATSAATEAGLELVDKGKAKELPGSQVIFEGPTLGQQEDQERKRL
CKAMTLCICYAASIGGTATLTGTGPNVVLLGQMNELFPDSKDLVNFASWFAFAFPNMLVMLLFAWLWL
QFVYMRFNFKKSWGCGLESKKNEKAALKVLQEEYRKLGPLSFAEINVLICFFLLVILWFSRDPGFMPG
WLTVAWVEGETKYVSDATVAIFVATLLFIVPSQKPKFNFRSQTEEERKTPFYPPPLLDWKVTQEKVPW
GIVLLLGGGFALAKGSEASGLSVWMGKQMEPLHAVPPAAITLILSLLVAVFTECTSNVATTTLFLPIF
ASMNHVPKSFCVLYGDVAVLSFRSLAPSASIRCTSCCPVP

A ClustalW comparison of the above protein sequences yields the following sequence alignment shown in Table 18B.

TABLE 18B
Comparison of the NOV18 protein sequences.

| | |
|---|---|
| NOV18a | MASALSYVSKFKSFVILFVTPLLLLPLVILMPAKVSCCAYVIILMAIYWCTEVIPLAVTS |
| NOV18b | MASALSYVSKFKSFVILFVTPLLLLPLVILMPAKVS-CAYVIILMAIYWCTEVIPLAVTS |
| NOV18c | MASALSYVSKFKSFVILFVTPLLLLPLVILMPAKFVRCAYVIILMAIYWCTEVIPLAVTS |
| NOV18d | MASALSYVSKFKSFVILFVTPLLLLPLVILMPAKFVRCAYVIILMAIYWCTEVIPLAVTS |
| NOV18e | MASALSYVSKFKSFVILFVTPLLLLPLVILMPAKFVRCAYVIILMAIYWCTEVIPLAVTS |
| NOV18a | LMPVLLFPLFQILDSRQVCVQYMKDTNMLFLGGLIVAVAVERWNLHKRIALRTLLWVGAK |
| NOV18b | LMPVLLFPLFQILDSRQVCVQYMKDTNMLFLGGLIVAVAVERWNLHKRIALRTLLWVGAK |
| NOV18c | LMPVLLFPLFQILDSRQVCVQYMKDTNMLFLGGLIVAVAVERWNLHKRIALRTLLWVGAK |
| NOV18d | LMPVLLFPLFQILDSRQVCVQYMKDTNMLFLGGLIVAVAVERWNLHKRIALRTLLWVGAK |
| NOV18e | LMPVLLFPLFQILDSRQVCVQYMKDTNMLFLGGLIVAVAVERWNLHKRIALRTLLWVGAK |
| NOV18a | PARLMLGFMGVTALLSMWISNTATTAMMVPIVEAILQQMEATSAATEAGLEGQGTTINNL |
| NOV18b | PARLMLGFMGVTALLSMWISNTATTAMMVPIVEAILQQMEATSAATEAGLEL----VD-- |
| NOV18c | PARLMLGFMGVTALLSMWISNTATTAMMVPIVEAILQQMEATSAATEAGLELVDK----- |
| NOV18d | PARLMLGFMGVTALLSMWISNTATTAMMVPIVEAILQQMEATSAATEAGLELVDK----- |
| NOV18e | PARLMLGFMGVTALLSMWISNTATTAMMVPIVEAILQQMEATSAATEAGLELVDK----- |
| NOV18a | NALEDDTVKAVLGGKCVAIISTYVKKVEKLQINNLMTPLKKLEKQEQQDLGPGIRPQDSA |
| NOV18b | --------KGK---------------------------AKELPGSQVIFEGPTLG---Q- |
| NOV18c | -------------GK-----------------------AKELPGSQVIFEGPTLG-Q--- |
| NOV18d | -------------GK-----------------------AKELPGSQVIFEGPTLG-Q--- |
| NOV18e | -------------GK-----------------------AKELPGSQVIFEGPTLG-Q--- |
| NOV18a | QCQEDQERKRLCKAMTLCICYAASIGGTATLTGTGPNVVLLGQMNELFPDSKDLVNFASW |
| NOV18b | -Q-EDQERKRLCKAMTLCICYAASIGGTATLTGTGPNVVLLGQMNELFPDSKDLVNFASW |
| NOV18c | --QEDQERKRLCKAMTLCICYAASIGGTATLTGTGPNVVLLGQMNELFPDSKDLVNFASW |
| NOV18d | --QEDQERKRLCKAMTLCICYAASIGGTATLTGTGPNVVLLGQMNELFPDSKDLVNFASW |
| NOV18e | --QEDQERKRLCKAMTLCICYAASIGGTATLTGTGPNVVLLGQMNELFPDSKDLVNFASW |
| NOV18a | FAFAFPNMLVMLLFAWLWLQFVYMFSSFKKSWGCGLESKKNEKAALKVLQEEYRKLGPLS |
| NOV18b | FAFAFPNMLVMLLFAWLWLQFVYMFSSFKKSWGCGLESKKNEKAALKVLQEEYRKLGPLS |
| NOV18c | FAFAFPNMLVMLLFAWLWLQFVYMRFNFKKSWGCGLESKKNEKAALKVLQEEYRKLGPLS |
| NOV18d | FAFAFPNMLVMLLFAWLWLQFVYMRFNFKKSWGCGLESKKNEKAALKVLQEEYRKLGPLS |
| NOV18e | FAFAFPNMLVMLLFAWLWLQFVYMRFNFKKSWGCGLESKKNEKAALKVLQEEYRKLGPLS |
| NOV18a | FAEINVLICFFLLVILWFSRDPGFMPGWLTVAWVEGETKSVSDATVAIFVATLLFIVPSQ |
| NOV18b | FAEINVLICFFLLVILWFSRDPGFMPGWLTVAWVEGETKYVSDATVAIFVATLLFIVPSQ |
| NOV18c | FAEINVLICFFLLVILWFSRDPGFMPGWLTVAWVEGETKYVSDATVAIFVATLLFIVPSQ |
| NOV18d | FAEINVLICFFLLVILWFSRDPGFMPGWLTVAWVEGETKYVSDATVAIFVATLLFIVPSQ |
| NOV18e | FAEINVLICFFLLVILWFSRDPGFMPGWLTVAWVEGETKYVSDATVAIFVATLLFIVPSQ |
| NOV18a | KPKFNFRSQTEEGKSPVLIAPPPLLDWKVTQEKVPWGIVLLLGGGFALAKGSEASGLSVW |
| NOV18b | KPKFNFRSQTEEGKSPVLIAPPPLLDWKVTQEKVPWGIVLLLGGGFALAKGSEASGLSVW |
| NOV18c | KPKFNFRSQTEEERK-TPFYPPPLLDWKVTQEKVPWGIVLLLGGGFALAKGSEASGLSVW |
| NOV18d | KPKFNFRSQTEEERK-TPFYPPPLLDWKVTQEKVPWGIVLLLGGGFALAKGSEASGLSVW |
| NOV18e | KPKFNFRSQTEEERK-TPFYPPPLLDWKVTQEKVPWGIVLLLGGGFALAKGSEASGLSVW |
| NOV18a | MGKQMEPLHAVPPAAITLILSLLVAVFTECTSNVATTTLFLPIFASMSR---SIGLNPLY |
| NOV18b | MGKQMEPLHAVPPAAITLILSLLVAVFTECTSNVATTTLFLPIFASMSR---SIGLNPLY |
| NOV18c | MGKQMEPLHAVPPAAITLILSLLVAVFTECTSNVATTTLFLPIFASMSR---SIGLNPLY |
| NOV18d | MGKQMEPLHAVPPAAITLILSLLVAVFTECTSNVATTTLFLPIFASMVKTGVIMNIIGVF |
| NOV18e | MGKQMEPLHAVPPAAITLILSLLVAVFTECTSNVATTTLFLPIFASMN------HVPKSF |
| NOV18a | IMLPCTLSASFAFMLPVATPPNAIVFTYGHLKVADMVKTGVIMNIIGVFCVFLAVNTWGR |
| NOV18b | IMLPCTLSASFAFMLPVATPPNAIVFTYGHLKVADMVKTGVIMNIIGVFCVFLAVNTWGR |
| NOV18c | IMLPCTLSASFAFMLPVATPPNAIVFTYGHLKVADMVKTGVIMNIIGVFCVFLAVNTWGR |
| NOV18d | CVFLAVNTWGRAIFDLDHFPDWANVTHIET------------------------------ |
| NOV18e | CVLYGD----VAVLSFRSLAPSASIRCTSCCPVP-------------------------- |
| NOV18a | AIFDLDHFPDWANVTHIET |
| NOV18b | AIFDLDHFPDWANVTHIET |
| NOV18c | AIFDLDHFPDWANVTHIET |
| NOV18d | ------------------- |
| NOV18e | ------------------- |
| NOV18a | (SEQ ID NO: 68) |
| NOV18b | (SEQ ID NO: 70) |
| NOV18c | (SEQ ID NO: 72) |
| NOV18d | (SEQ ID NO: 74) |
| NOV18e | (SEQ ID NO: 76) |

Further analysis of the NOV18a protein yielded the following properties shown in Table 18C.

TABLE 18C
Protein Sequence Properties NOV18a

| SignalP analysis: | Cleavage site between residues 39 and 40 | | | | |
|---|---|---|---|---|---|
| PSORT II analysis: | | | | | |
| | PSG: a new signal peptide prediction method | | | | |
| | N-region: length 10; pos. chg 1; neg. chg 0 | | | | |
| | H-region: length 1; peak value 5.97 | | | | |
| | PSG score: 1.57 | | | | |
| | GvH: von Heijne's method for signal seq. recognition | | | | |
| | GvH score (threshold: −2.1): −2.36 | | | | |
| | possible cleavage site: between 30 and 31 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 11 | | | | |
| | INTEGRAL | Likelihood = | −12.21 | Transmembrane | 14-30 |
| | INTEGRAL | Likelihood = | −3.61 | Transmembrane | 35-51 |
| | INTEGRAL | Likelihood = | −4.99 | Transmembrane | 53-69 |
| | INTEGRAL | Likelihood = | −1.75 | Transmembrane | 124-140 |
| | INTEGRAL | Likelihood = | −4.19 | Transmembrane | 309-325 |
| | INTEGRAL | Likelihood = | −8.01 | Transmembrane | 359-375 |
| | INTEGRAL | Likelihood = | −6.26 | Transmembrane | 401-417 |
| | INTEGRAL | Likelihood = | 0.21 | Transmembrane | 453-469 |
| | INTEGRAL | Likelihood = | −7.11 | Transmembrane | 491-507 |
| | INTEGRAL | Likelihood = | −1.70 | Transmembrane | 538-554 |
| | INTEGRAL | Likelihood = | −7.43 | Transmembrane | 576-592 |

| PERIPHERAL | Likelihood = | 0.74 (at 89) | |
|---|---|---|---|
| ALOM score: −12.21 (number of TMSs: 11) | | | |
| MTOP: Prediction of membrane topology (Hartmann et al.) | | | |
| Center position for calculation: 21 | | | |
| Charge difference: −2.0 C(1.0)-N(3.0) | | | |
| N >= C: N-terminal side will be inside | | | |
| >>> membrane topology: type 3a | | | |
| MITDISC: discrimination of mitochondrial targeting seq | | | |
| R content: | 0 | Hyd Moment(75): | 1.18 |
| Hyd Moment (95): | 3.95 | G content: | 0 |
| D/E content: | 1 | S/T content: | 7 |

| Score: −3.82 | |
|---|---|
| Gavel: prediction of cleavage sites for mitochondrial preseq | |
| cleavage site motif not found | |
| NUCDISC: discrimination of nuclear localization signals | |
| pat4: none | |
| pat7: none | |
| bipartite: none | |
| content of basic residues: 7.6% | |
| NLS Score: −0.47 | |
| KDEL: ER retention motif in the C-terminus: none | |
| ER Membrane Retention Signals: none | |
| SKL: peroxisomal targeting signal in the C-terminus: none | |
| PTS2: 2nd peroxisomal targeting signal: none | |
| VAC: possible vacuolar targeting motif: none | |
| RNA-binding motif: none | |
| Actinin-type actin-binding motif: | |
| type 1: none | |
| type 2: none | |
| NMYR: N-myristoylation pattern: none | |
| Prenylation motif: none | |
| memYQRL: transport motif from cell surface to Golgi: none | |
| Tyrosines in the tail: none | |
| Dileucine motif in the tail: none | |
| checking 63 PROSITE DNA binding motifs: none | |
| checking 71 PROSITE ribosomal protein motifs: none | |
| checking 33 PROSITE prokaryotic DNA binding motifs: none | |
| NNCN: Reinhardt's method for Cytoplasmic/Nuclear discrimination | |
| Prediction: cytoplasmic | |
| Reliability: 94.1 | |
| COIL: Lupas's algorithm to detect coiled-coil regions | |
| total: 0 residues | |
| Final Results (k = 9/23): | |
| 66.7%: | endoplasmic reticulum |
| 22.2%: | mitochondrial |
| 11.1%: | nuclear |

>> prediction for CG57758-02 is end (k=9)

A search of the NOV18a protein against the Geneseq database, a proprietary database that contains sequences published in patents and patent publication, yielded several homologous proteins shown in Table 18D.

TABLE 18D
Geneseq Results for NOV18a

| | | NOV18a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| AAU79946 | Human transporter protein | 1 . . . 616 | 535/617 (86%) | 0.0 |
| | sequence - Homo sapiens, 568 aa. | 1 . . . 568 | 542/617 (87%) | |
| | [US2002019028-A1, 14 FEB. | | | |
| | 2002] | | | |
| AAB23625 | Human secreted protein SEQ ID | 10 . . . 614 | 253/623 (40%) | e−141 |
| | NO: 50 - Homo sapiens, 627 aa. | 9 . . . 623 | 395/623 (62%) | |
| | [WO200049134-A1, 24 AUG. | | | |
| | 2000] | | | |
| AAB36158 | Novel human transporter protein | 10 . . . 614 | 253/623 (40%) | e−141 |
| | SEQ ID NO: 2 - Homo sapiens, | 9 . . . 623 | 395/623 (62%) | |
| | 627 aa. [WO200065055-A2, 02 | | | |
| | NOV. 2000] | | | |
| ABB97450 | Novel human protein SEQ ID NO: | 10 . . . 614 | 253/623 (40%) | e−141 |
| | 718 - Homo sapiens, 627 aa. | 9 . . . 623 | 395/623 (62%) | |
| | [WO200222660-A2, 21 MAR. | | | |
| | 2002] | | | |
| AAB42213 | Human ORFX ORF1977 | 10 . . . 614 | 253/623 (40%) | e−141 |
| | polypeptide sequence SEQ ID | 9 . . . 623 | 395/623 (62%) | |
| | NO: 3954 - Homo sapiens, 627 aa. | | | |
| | [WO200058473-A2, 05 OCT. | | | |
| | 2000] | | | |

In a BLAST search of public sequence databases, the NOV18a protein was found to have homology to the proteins shown in the BLASTP data in Table 18E.

TABLE 18E
Public BLASTP Results for NOV18a

| | | NOV18a | Identities/ | |
|---|---|---|---|---|
| Protein | | Residues/ | Similarities | |
| Accession | | Match | for the Matched | Expect |
| Number | Protein/Organism/Length | Residues | Portion | Value |
| AAN86530 | Na+-coupled citrate transporter | 1 . . . 616 | 537/617 (87%) | 0.0 |
| | protein - Homo sapiens (Human), | 1 . . . 568 | 544/617 (88%) | |
| | 568 aa. | | | |
| Q8CJ44 | Sodium-coupled citrate transporter | 1 . . . 616 | 422/620 (68%) | 0.0 |
| | - Rattus norvegicus (Rat), 572 aa. | 1 . . . 572 | 484/620 (78%) | |
| O57661 | Intestinal sodium/LITHIUM- | 1 . . . 612 | 355/619 (57%) | 0.0 |
| | dependent dicarboxylate | 1 . . . 619 | 472/619 (75%) | |
| | transporter (NA(+)/dicarboxylate | | | |
| | cotransporter) - Xenopus laevis | | | |
| | (African clawed frog), 622 aa. | | | |
| AAH44437 | Similar to solute carrier family 13, | 5 . . . 612 | 330/635 (51%) | 0.0 |
| | member 2 - Brachydanio rerio | 10 . . . 605 | 443/635 (68%) | |
| | (Zebrafish) (Danio rerio), 613 aa. | | | |
| AAO27449 | Sodium dicarboxylate Co- | 11 . . . 611 | 324/602 (53%) | 0.0 |
| | transporter - Didelphis marsupialis | 11 . . . 587 | 439/602 (72%) | |
| | virginiana (North American | | | |
| | opossum), 605 aa. | | | |

PFam analysis predicts that the NOV18a protein contains the domains shown in the Table 18F.

TABLE 18F
Domain Analysis of NOV18a

| | | Identities/ | |
|---|---|---|---|
| | | Similarities | |
| | NOV18a | for the Matched | Expect |
| Pfam Domain | Match Region | Region | Value |
| Na_sulph_symp | 6 . . . 602 | 163/647 (25%) | 3.5e−134 |
| | | 447/647 (69%) | |

### Example 19

The NOV19 clone was analyzed, and the nucleotide and encoded polypeptide sequences are shown in Table 19A.

TABLE 19A
NOV19 Sequence Analysis

| | | |
|---|---|---|
| NOV19a, CG59693-01 | SEQ ID NO: 77 | 972 bp |
| DNA Sequence | ORF Start: ATG at 1 | ORF Stop: end of sequence |

ATGGATTCGAAATATCAGTGTGTGAAGCTGAATGATGGTCACTTCATGCCTGTCCTGGGATTTGGCAC
CTATGCGCCTGCAGAGGTTCCTAAAAGTAAAGCTTTAGAGGCCACCAAATTGGCAATTGAAGCTGGCT
TCCGCCATATTGATTCTGCTCATTTATACAATAATGAGGAGCAGGTTGGACTGGCCATCCGAAGCAAG
ATTGCAGATGGCAGTGTGAAGAGAGAAGACATATTCTACACTTCAAAGCTTTGGTGCAATTCCCATCG
ACCAGAGTTGGTCCGACCAGCCTTGGAAAGGTCACTGAAAAATCTTCAATTGGATTATGTTGACCTCT
ACCTTATTCATTTTCCAGTGTCTGTAAAGCCAGGTGAGGAAGTGATCCCAAAAGATGAAAATGGAAAA
ATACTATTTGACACAGTGGATCTCTGTGCCACGTGGGAGGCCGTGGAGAAGTGTAAAGATGCAGGATT
GGCCAAGTCCATCGGGGTGTCCAACTTCAACCGCAGGCAGCTGGAGATGATCCTCAACAAGCCAGGGC
TCAAGTACAAGCCTGTCTGCAACCAGGTGGAATGTCATCCTTACTTCAACCAGAGAAAACTGCTGGAT
TTCTGCAAGTCAAAAGACATTGTTCTGGTTGCCTATAGTGCTCTGGGATCCCACCGAGAAGAACCATG
GGTGGACCCGAACTCCCCGGTGCTCTTGGAGGACCCAGTCCTTTGTGCCTTGGCAAAAAAGCACAAGC
GAACCCCAGCCCTGATTGCCCTGCGCTACCAGCTACAGCGTGGGGTTGTGGTCCTGGCCAAGAGCTAC
AATGAGCAGCGCATCAGACAGAACGTGCAGGTGTTTGAATTCCAGTTGACTTCAGAGGAGATGAAAGC
CATAGATGGCCTAAACAGAAATGTGCGATATTTGACCCTTGATATTTTTGCTGGCCCCCCTAATTATC
CATTTTCTGATGAATATTAA

| | | | |
|---|---|---|---|
| NOV19a, CG59693-01 | SEQ ID NO: 78 | 323 aa | MW at 36787.9 kD |
| Protein Sequence | | | |

MDSKYQCVKLNDGHFMPVLGFGTYAPAEVPKSKALEATKLAIEAGFRHIDSAHLYNNEEQVGLAIRSK
IADGSVKREDIFYTSKLWCNSHRPELVRPALERSLKNLQLDYVDLYLIHFPVSVKPGEEVIPKDENGK
ILFDTVDLCATWEAVEKCKDAGLAKSIGVSNFNRRQLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKSY
NEQRIRQNVQVFEFQLTSEEMKAIDGLNRNVRYLTLDIFAGPPNYPFSDEY

| | | |
|---|---|---|
| NOV19b, CG59693-02 | SEQ ID NO: 79 | 983 bp |
| DNA Sequence | ORF Start: ATG at 30 | ORF Stop: end of sequence |

ATGGATTCGATATCAGTGTGTGAAGCTGAATGATGGTCACTTCGTGCCTGTCCTGGGATTTGGCACCT
ATGCGCCTGCAGAGGTTACTCCCCCAGGTTCCTAAAAGTAAAGCTTTAGAGGCCACCAAATTGGCAAT
TGAAGCTGGCTTCCGCCATATTGATTCTGCTCATTTATACAATAATGAGGAGCAGGTTGGACTGGCCA
TCCGAAGCAAGATTGCAGATGGCAGTGTGAAGAGAGAAGACATATTCTACACTTCAAAGCTTTGGTGC
AATTCCCATCGACCAGAGTTGGTCCGACCAGCCTTGGAAAGGTCACTGAAAAATCTTCAATTGGATTA
TGTTGACCTCTACCTTATTCATTTTCCAGTGTCTGTAAAGCCAGGTGAGGAAGTGATCCCAAAAGATG
AAAGTGGAAAAATACTATTTGACACAGTGGATCTCTGTGCCACGTGGGAGGCCGTGGAGAAGTGTAAA
GATGCAGGATTGGCCAAGTCCATCGGGGTGTCCAACTTCAACCGCAGGCAGCTGGAGATGATCCTCAA
CAAGCCAGGGCTCAAGTACAAGCCTGTCTGCAACCAGGTGGAATGTCATCCTTACTTCAACCAGAGAA
AACTGCTGGATTTCTGCAAGTCAAAAGACATTGTTCTGGTTGCCTATAGTGCTCTGGGATCCCACCGA
GAAGAACCATGGGTGGACCCGAACTCCCCGGTGCTCTTGGAGGACCCAGTCCTTTGTGCCTTGGCAAA
AAAGCACAAGCGAACCCCAGCCCTGGTTGCCCTGCGCTACCAGCTACAGCGTGGGGTTGTGGTCCTGG
CCAAGAGCTACAATGAGCAGCGCATCAGACAGAACGTGCAGGTGTTTGAATTCCAGTTGACTTCAGAG
GAGATGAAAGCCATAGATGGCCTAAACAGAAATGTGCGATATTTGACCCTTGATATTTTTGCTGGCCC
CCCTAATTATCCATTTTCTGATGAATATTAA

| | | | |
|---|---|---|---|
| NOV19b, CG59693-02 | SEQ ID NO: 80 | 317 aa | MW at 36217.5 kD |
| Protein Sequence | | | |

MMVTSCLSWDLAPMRLQRLLPQVPKSKALEATKLAIEAGFRHIDSAHLYNNEEQVGLAIRSKIADGSV
KREDIFYTSKLWCNSHRPELVRPALERSLKNLQLDYVDLYLIHFPVSVKPGEEVIPKDESGKILFDTV
DLCATWEAVEKCKDAGLAKSIGVSNFNRRQLEMILNKPGLKYKPVCNQVECHPYFNQRKLLDFCKSKD
IVLVAYSALGSHREEPWVDPNSPVLLEDPVLCALAKKHKRTPALVALRYQLQRGVVVLAKSYNEQRIR
QNVQVFEFQLTSEEMKAIDGLNRNVRYLTLDIFAGPPNYPFSDEY

| | | |
|---|---|---|
| NOV19c, CG59693-03 | SEQ ID NO: 81 | 972 bp |
| DNA Sequence | ORF Start: ATG at 1 | ORF Stop: end of sequence |

ATGGATTCGAAATATCAGTGTGTGAAGCTGAATGATGGTCACTTCATGCCTGTCCTGGCATTTGGCAC
CTATGCGCCTGCAGAGGTTCCTAAAAGTAAAGCTTTAGAGGCCACCAAATTGGCAATTGAAGCTGGCT
TCCGCCATATTGATTCTGCTCATTTATACAATAATGAGGAGCAGGTTGGACTGGCCATCCGAAGCAAG
ATTGCAGATGGCAGTGTGAAGAGAGAAGACATATTCTACACTTCAAAGCTTTGGTGCAATTCCCATCG
ACCAGAGTTGGTCCGACCAGCCTTGGAAAGGTCACTGAAAAATCTTCAATTGGATTATGTTGACCTCT
ACCTTATTCATTTTCCAGTGTCTGTAAAGCCAGGTGAGGAAGTGATCCCAAAAGATGAAAATGGAAAA
ATACTATTTGACACAGTGGATCTCTGTGCCACGTGGGAGGCCGTGGAGAAGTGTAAAGATGCAGGATT
GGCCAAGTCCATCGGGGTGTCCAACTTCAACCGCAGGCAGCTGGAGATGATCCTCAACAAGCCAGGGC
TCAAGTACAAGCCTGTCTGCAACCAGGTGGAATGTCATCCTTACTTCAACCAGAGAAAACTGCTGGAT
TTCTGCAAGTCAAAAGACATTGTTCTGGTTGCCTATAGTGCTCTGGGATCCCACCGAGAAGAACCATG
GGTGGACCCGAACTCCCCGGTGCTCTTGGAGGACCCAGTCCTTTGTGCCTTGGCAAAAAAGCACAAGC
GAACCCCAGCCCTGATTGCCCTGCGCTACCAGCTACAGCGTGGGGTTGTGGTCCTGGCCAAGAGATAC
AATGAGCAGCGCATCAGACAGAACGTGCAGGTGTTTGAATTCCAGTTGACTTCAGAGGAGATGAAAGC
CATAGATGGCCTAAACAGAAATGTGCGATATTTGACCCTTGATATTTTTGCTGGCCCCCCTAATTATC
CATTTTCTGATGAATATTAA

| | | | |
|---|---|---|---|
| NOV19c, CG59693-03 | SEQ ID NO: 82 | 323 aa | MW at 36857.0 kD |
| Protein Sequence | | | |

MDSKYQCVKLNDGHFMPVLGFGTYAPAEVPKSKALEATKLAIEAGFRHIDSAHLYNNEEQVGLAIRSK
IADGSVKREDIFYTSKLWCNSHRPELVRPALERSLKNLQLDYVDLYLIHFPVSVKPGEEVIPKDENGK
ILFDTVDLCATWEAVEKCKDAGLAKSIGVSNFNRRQLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKRY
NEQRIRQNVQVFEFQLTSEEMKAIDGLNRNVRYLTLDIFAGPPNYPFSDEY

| | | |
|---|---|---|
| NOV19d, CG59693-04 | SEQ ID NO: 83 | 994 bp |
| DNA Sequence | ORF Start: ATG at 16 | ORF Stop: end of sequence |

GCCAGATCTCCCACCATGGATTCGAAATATCAGTGTGTGAAGCTGAATGATGGTCACTTCATGCCTGT
CCTGGGATTTGGCACCTATGCGCCTGCAGAGGTTCCTAAAAGTAAAGCTTTAGAGGCCACCAAATTGG
CAATTGAAGCTGGCTTCCGCCATATTGATTCTGCTCATTTATACAATAATGAGGAGCAGGTTGGACTG
GCCATCCGAAGCAAGATTGCAGATGGCAGTGTGAAGAGAGAAGACATATTCTACACTTCAAAGCTTTG
GTGCAATTCCCATCGACCAGAGTTGGTCCGACCAGCCTTGGAAAGGTCACTGAAAAATCTTCAATTGG
ATTATGTTGACCTCTACCTTATTCATTTTCCAGTGTCTGTAAAGCCAGGTGAGGAAGTGATCCCAAAA
GATGAAAATGGAAAAATACTATTTGACACAGTGGATCTCTGTGCCACGTGGGAGGCCGTGGAGAAGTG
TAAAGATGCAGGATTGGCCAAGTCCATCGGGGTGTCCAACTTCAACCGCAGGCAGCTGGAGATGATCC
TCAACAAGCCAGGGCTCAAGTACAAGCCTGTCTGCAACCAGGTGGAATGTCATCCTTACTTCAACCAG
AGAAAACTGCTGGATTTCTGCAAGTCAAAAGACATTGTTCTGGTTGCCTATAGTGCTCTGGGATCCCA
CCGAGAAGAACCATGGGTGGACCCGAACTCCCCGGTGCTCTTGGAGGACCCAGTCCTTTGTGCCTTGG
CAAAAAAGCACAAGCGAACCCCAGCCCTGATTGCCCTGCGCTACCAGCTACAGCGTGGGGTTGTGGTC
CTGGCCAAGAGCTACAATGAGCAGCGCATCAGACAGAACGTGCAGGTGTTTGAATTCCAGTTGACTTC
AGAGGAGATGAAAGCCATAGATGGCCTAAACAGAAATGTGCGATATTTGACCCTTGATATTTTTGCTG
GCCCCCCTAATTATCCATTTTCTGATGAATATCTCGAGGGTG

| | | | |
|---|---|---|---|
| NOV19d, CG59693-04 | SEQ ID NO: 84 | 321 aa | MW at 36495.6 kD |
| Protein Sequence | | | |

MDSKYQCVKLNDGHFMPVLGFGTYAPAEVPKSKALEATKLAIEAGFRHIDSAHLYNNEEQVGLAIRSK
IADGSVKREDIFYTSKLWCNSHRPELVRPALERSLKNLQLDYVDLYLIHFPVSVKPGEEVIPKDENGK
ILFDTVDLCATWEAVEKCKDAGLAKSIGVSNFNRRQLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKSY
NEQRIRQNVQVFEFQLTSEEMKAIDGLNRNVRYLTLDIFAGPPNYPFSD

| | | |
|---|---|---|
| NOV19e, CG59693-05 | SEQ ID NO: 85 | 1219 bp |
| DNA Sequence | ORF Start: ATG at 24 | ORF Stop: end of sequence |

TGCTAACCAGGCCAGTGACAGAAATGGATTCGAAATACCAGTGTGTGAAGCTGAATGATGGTCACTTC
ATGCCTGTCCTGGGATTTGGCACCTATGCGCCTGCAGAGGTTCCTAAAAGTAAAGCTCTAGAGGCCGT
CAAATTGGCAATAGAAGCCGGGTTCCACCATATTGATTCTGCACATGTTTACAATAATGAGGAGCAGG
TTGGACTGGCCATCCGAAGCAAGATTGCAGATGGCAGTGTGAAGAGAGAAGACATATTCTACACTTCA
AAGCTTTGGAGCAATTCCCATCGACCAGAGTTGGTCCGACCAGCCTTGGAAAGGTCACTGAAAAATCT
TCAATTGGACTATGTTGACCTCTATCTTATTCATTTTCCAGTGTCTGTAAAGCCAGGTGAGGAAGTGA
TCCCAAAAGATGAAAATGGAAAAATACTATTTGACACAGTGGATCTCTGTGCCACGTGGGAGGCCATG
GAGAAGTGTAAAGATGCAGGATTGGCCAAGTCCATCGGGGTGTCCAACTTCAACCACAGGCTGCTGGA
GATGATCCTCAACAAGCCAGGGCTCAAGTACAAGCCTGTCTGCAACCAGGTGGAATGTCATCCTTACT
TCAACCAGAGAAAACTGCTGGATTTCTGCAAGTCAAAAGACATTGTTCTGGTTGCCTATAGTGCTCTG
GGATCCCATCGAGAAGAACCATGGGTGGACCCGAACTCCCCGGTGCTCTTGGAGGACCCAGTCCTTTG
TGCCTTGGCAAAAAAGCACAAGCGAACCCCAGCCCTGATTGCCCTGCGCTACCAGCTGCAGCGTGGGG
TTGTGGTCCTGGCCAAGAGCTACAATGAGCAGCGCATCAGACAGAACGTGCAGGTGTTTGAATTCCAG
TTGACTTCAGAGGAGATGAAAGCCATAGATGGCCTAAACAGAAATGTGCGATATTTGACCCTTGATAT
TTTTGCTGGCCCCCCTAATTATCCATTTTCTGATGAATATTAACATGGAGGGCATTGCATGAGGTCTG
CCAGAAGGCCCTGCGTGTGGATGGTGACACAGAGGATGGCTCTATGCTGGTGACTGGACACATCGCCT
CTGGTTAAATCTCTCCTGCTTGGCGACTTCAGTAAGCTACAGCTAAGCCCATCGGCCGGAAAAGAAAG
ACAATAATTTTGTTTTTTCATTTTGAAAAAATTAAATGCTCTCTCCTAAAGATTCTTCACCTA

| | | | |
|---|---|---|---|
| NOV19e, CG59693-05 | SEQ ID NO: 86 | 323 aa | MW at 36734.9 kD |
| Protein Sequence | | | |

MDSKYQCVKLNDGHFMPVLGFGTYAPAEVPKSKALEAVKLAIEAGFHHIDSAHVYNNEEQVGLAIRSK
IADGSVKREDIFYTSKLWSNSHRPELVRPALERSLKNLQLDYVDLYLIHFPVSVKPGEEVIPKDENGK
ILFDTVDLCATWEAMEKCKDAGLAKSIGVSNFNHRLLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKSY
NEQRIRQNVQVFEFQLTSEEMKAIDGLNRNVRYLTLDIFAGPPNYPFSDEY

| | | |
|---|---|---|
| NOV19f, CG59693-06 | SEQ ID NO: 87 | 1001 bp |
| DNA Sequence | ORF Start: at 11 | ORF Stop: end of sequence |

CATCTAGGCCACCATGGCCATGGATTCGAAATATCAGTGTGTGAAGCTGAATGATGGTCACTTCATGC
CTGTCCTGGGATTTGGCACCTATGCGCCTGCAGAGGTTCCTAAAAGTAAAGCTTTAGAGGCCACCAAA
TTGGCAATTGAAGCTGGCTTCCGCCATATTGATTCTGCTCATTTATACAATAATGAGGAGCAGGTTGG
ACTGGCCATCCGAAGCAAGATTGCAGATGGCAGTGTGAAGAGAGAAGACATATTCTACACTTCAAAGC
TTTGGTGCAATTCCCATCGACCAGAGTTGGTCCGACCAGCCTTGGAAAGGTCACTGAAAAATCTTCAA
TTGGATTATGTTGACCTCTACCTTATTCATTTTCCAGTGTCTGTAAAGCCAGGTGAGGAAGTGATCCC
AAAAGATGAAAATGGAAAAATACTATTTGACACAGTGGATCTCTGTGCCACGTGGGAGGCCGTGGAGA
AGTGTAAAGATGCAGGATTGGCCAAGTCCATCGGGGTGTCCAACTTCAACCGCAGGCAGCTGGAGATG
ATCCTCAACAAGCCAGGGCTCAAGTACAAGCCTGTCTGCAACCAGGTGGAATGTCATCCTTACTTCAA
CCAGAGAAAACTGCTGGATTTCTGCAAGTCAAAAGACATTGTTCTGGTTGCCTATAGTGCTCTGGGAT
CCCACCGAGAAGAACCATGGGTGGACCCGAACTCCCCGGTGCTCTTGGAGGACCCAGTCCTTTGTGCC
TTGGCAAAAAAGCACAAGCGAACCCCAGCCCTGATTGCCCTGCGCTACCAGCTACAGCGTGGGGTTGT
GGTCCTGGCCAAGAGCTACAATGAGCAGCGCATCAGACAGAACGTGCAGGTGTTTGAATTCCAGTTGA
CTTCAGAGGAGATGAAAGCCATAGATGGCCTAAACAGAAATGTGCGATATTTGACCCTTGATATTTTT
GCTGGCCCCCCTAATTATCCATTTTCTGATGAATATTAAACGCGTGATC

| | | | |
|---|---|---|---|
| NOV19f, CG59693-06 | SEQ ID NO: 88 | 324 aa | MW at 36799.0 kD |
| Protein Sequence | | | |

TMAMDSKYQCVKLNDGHFMPVLGFGTYAPAEVPKSKALEATKLAIEAGFRHIDSAHLYNNEEQVGLAI
RSKIADGSVKREDIFYTSKLWCNSHRPELVRPALERSLKNLQLDYVDLYLIHFPVSVKPGEEVIPKDE
NGKILFDTVDLCATWEAVEKCKDAGLAKSIGVSNFNRRQLEMILNKPGLKYKPVCNQVECHPYFNQRK
LLDFCKSKDIVLVAYSALGSHREEPWVDPNSPVLLEDPVLCALAKKHKRTPALIALRYQLQRGVVVLA
KSYNEQRIRQNVQVFEFQLTSEEMKAIDGLNRNVRYLTLDIFAGPPNYPFSD

| | | |
|---|---|---|
| NOV19g, CG59693-07 | SEQ ID NO: 89 | 1012 bp |
| DNA Sequence | ORF Start: at 1 | ORF Stop: end of sequence |

GCCGGTACCACCATGGGCCACCATCACCACCATCACGATTCGAAATATCAGTGTGTGAAGCTGAATGA
TGGTCACTTCATGCCTGTCCTGCGATTTGGCACCTATGCGCCTGCAGAGGTTCCTAAAAGTAAAGCTT
TAGAGGCCACCAAATTGGCAATTGAAGCTGGCTTCCGCCATATTGATTCTGCTCATTTATACAATAAT
GAGGAGCAGGTTGGACTGGCCATCCGAAGCAAGATTGCAGATGGCAGTGTGAAGAGAGAAGACATATT
CTACACTTCAAAGCTTTGGTGCAATTCCCATCGACCAGAGTTGGTCCGACCAGCCTTGGAAAGGTCAC
TGAAAAATCTTCAATTGGATTATGTTGACCTCTACCTTATTCATTTTCCAGTGTCTGTAAAGCCAGGT
GAGGAAGTGATCCCAAAAGATGAAAATGGAAAAATACTATTTGACACAGTGGATCTCTGTGCCACGTG
GGAGGCCGTGGAGAAGTGTAAAGATGCAGGATTGGCCAAGTCCATCGGGGTGTCCAACTTCAACCGCA
GGCAGCTGGAGATGATCCTCAACAAGCCAGGGCTCAAGTACAAGCCTGTCTGCAACCAGGTGGAATGT
CATCCTTACTTCAACCAGAGAAAACTGCTGGATTTCTGCAAGTCAAAAGACATTGTTCTGGTTGCCTA
TAGTGCTCTGGGATCCCACCGAGAAGAACCATGGGTGGACCCGAACTCCCCGGTGCTCTTGGAGGACC
CAGTCCTTTGTGCCTTGGCAAAAAAGCACAAGCGAACCCCAGCCCTGATTGCCCTGCGCTACCAGCTA
CAGCGTGGGGTTGTGGTCCTGGCCAAGAGCTACAATGAGCAGCGCATCAGACAGAACGTGCAGGTGTT
TGAATTCCAGTTGACTTCAGAGGAGATGAAAGCCATAGATGGCCTAAACAGAAATGTGCGATATTTGA
CCCTTGATATTTTTGCTGGCCCCCCTAATTATCCATTTTCTGATGAATATCTCGAGGGTG

| | | | |
|---|---|---|---|
| NOV19g, CG59693-07 | SEQ ID NO: 90 | 337 aa | MW at 38297.5 kD |
| Protein Sequence | | | |

AGTTMGHHHHHHDSKYQCVKLNDGHFMPVLGFGTYAPAEVPKSKALEATKLAIEAGFRHIDSAHLYNN
EEQVGLAIRSKIADGSVKREDIFYTSKLWCNSHRPELVRPALERSLKNLQLDYVDLYLIHFPVSVKPG
EEVIPKDENGKILFDTVDLCATWEAVEKCKDAGLAKSIGVSNFNRRQLEMILNKPGLKYKPVCNQVEC
HPYFNQRKLLDFCKSKDIVLVAYSALGSHREEPWVDPNSPVLLEDPVLCALAKKHKRTPALIALRYQL
QRGVVVLAKSYNEQRIRQNVQVFEFQLTSEEMKAIDGLNRNVRYLTLDIFAGPPNYPFSDEYLEG

| | | |
|---|---|---|
| NOV19h, CG59693-08 | SEQ ID NO: 91 | 1225 bp |
| DNA Sequence | ORF Start: ATG at 24 | ORF Stop: end of sequence |

TGCTAACCAGGCCAGTGACAGAAATGGATTCGAAATACCAGTGTGTGAAGCTGAATGATGGTCACTTC
ATGCCTGTCCTGGGATTTGGCACCTATGCGCCTGCAGAGGTTCCTAAAAGTAAAGCTCTAGAGGCCGT
CAAATTGGCAATAGAAGCCGGGTTCCACCATATTGATTCTGCACATGTTTACAATAATGAGGAGCAGG
TTGGACTGGCCATCCGAAGCAAGATTGCAGATGGCAGTGTGAAGAGAGAAGACATATTCTACACTTCA
AAGCTTTGGAGCAATTCCCATCGACCAGAGTTGGTCCGACCAGCCTTGGAAAGGTCACTGAAAAATCT
TCAATTGGACTATGTTGACCTCTATCTTATTCATTTTCCAGTGTCTGTAAAGCCAGGTGAGGAAGTGA
TCCCAAAAGATGAAAATGGAAAAATACTATTTGACACAGTGGATCTCTGTGCCACGTGGGAGGCCATG
GAGAAGTGTAAAGATGCAGGATTGGCCAAGTCCATCGGGGTGTCCAACTTCAACCACAGGCTGCTGGA
GATGATCCTCAACAAGCCAGGGCTCAAGTACAAGCCTGTCTGCAACCAGGTGGAATGTCATCCTTACT
TCAACCAGAGAAAACTGCTGGATTTCTGCAAGTCAAAAGACATTGTTCTGGTTGCCTATAGTGCTCTG
GGATCCCATCGAGAAGAACCATGGGTGGACCCGAACTCCCCGGTGCTCTTGGAGGACCCAGTCCTTTG
TGCCTTGGCAAAAAAGCACAAGCGAACCCCAGCCCTGATTGCCCTGCGCTACCAGCTGCAGCGTGGGG
TTGTGGTCCTGGCCAAGAGCTACAATGAGCAGCGCATCAGACAGAACGTGCAGGTGTTTGAATTCCAG
TTGACTTCAGAGGAGATGAAAGCCATAGATGGCCTAAACAGAAATGTGCGATATTTGACCCTTGATAT
TTTTGCTGGCCCCCCTAATTATCCATTTTCTGATGAATATTAACATGGAGGGCATTGCATGAGGTCTG
CCAGAAGGCCCTGCGTGTGGATGGTGACACAGAGGATGGCTCTATGCTGGTGACTGGACACATCGCCT
CTGGTTAAATCTCTCCTGCTTGGCGACTTCAGTAAGCTACAGCTAAGCCCATCGGCCGGAAAAGAAAG
ACAATAATTTTGTTTTTTCATTTTGAAAAAATTAAATGCTCTCTCCTAAAGATTCTTCACCTAAAAAA
A

| | | | |
|---|---|---|---|
| NOV19h, CG59693-08 | SEQ ID NO: 92 | 323 aa | MW at 36734.9 kD |
| Protein Sequence | | | |

MDSKYQCVKLNDGHFMPVLGFGTYAPAEVPKSKALEAVKLAIEAGFHHIDSAHVYNNEEQVGLAIRSK
IADGSVKREDIFYTSKLWSNSHRPELVRPALERSLKNLQLDYVDLYLIHFPVSVKPGEEVIPKDENGK
ILFDTVDLCATWEAMEKCKDAGLAKSIGVSNFNHRLLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKSY
NEQRIRQNVQVFEFQLTSEEMKAIDGLNRNVRYLTLDIFAGPPNYPFSDEY

| | | |
|---|---|---|
| NOV19i, CG59693-09 | SEQ ID NO: 93 | 996 bp |
| DNA Sequence | ORF Start: ATG at 16 | ORF Stop: end of sequence |

CACCGCGGCCGCACCATGGATTCGAAATATCAGTGTGTGAAGCTGAATGATGGTCACTTCATGCCTGT
CCTGGGATTTGGCACCTATGCGCCTGCAGAGGTTCCTAAAAGTAAAGCTTTAGAGGCCACCAAATTGG
CAATTGAAGCTGGCTTCCGCCATATTGATTCTGCTCATTTATACAATAATGAGGAGCAGGTTCGACTG
GCCATCCGAAGCAAGATTGCAGATGGCAGTGTGAAGAGAGAAGACATATTCTACACTTCAAAGCTTTG
GTGCAATTCCCATCGACCAGAGTTGGTCCGACCAGCCTTGGAAAGGTCACTGAAAAATCTTCAATTGG
ATTATGTTGACCTCTACCTTATTCATTTTCCAGTGTCTGTAAAGCCAGGTGAGGAAGTGATCCCAAAA
GATGAAAATGGAAAAATACTATTTGACACAGTGGATCTCTGTGCCACGTGGGAGGCCGTGGAGAAGTG
TAAAGATGCAGGATTGGCCAAGTCCATCGGGGTGTCCAACTTCAACCGCAGGCAGCTGGAGATGATCC
TCAACAAGCCAGGGCTCAAGTACAAGCCTGTCTGCAACCAGGTGGAATGTCATCCTTACTTCAACCAG
AGAAAACTGCTGGATTTCTGCAAGTCAAAAGACATTGTTCTGGTTGCCTATAGTGCTCTGGGATCCCA
CCGAGAAGAACCATGGGTGGACCCGAACTCCCCGGTGCTCTTGGAGGACCCAGTCCTTTGTGCCTTGG
CAAAAAAGCACAAGCGAACCCCAGCCCTGATTGCCCTGCGCTACCAGCTACAGCGTGGGGTTGTGGTC
CTGGCCAAGAGCTACAATGAGCAGCGCATCAGACAGAACGTGCAGGTGTTTGAATTCCAGTTGACTTC
AGAGGAGATGAAAGCCATAGATGGCCTAAACAGAAATGTGCGATATTTGACCCTTGATATTTTTGCTG
GCCCCCCTAATTATCCATTTTCTGATGAATATTAGGTCGACGGC

| | | | |
|---|---|---|---|
| NOV19i, CG59693-09 | SEQ ID NO: 94 | 323 aa | MW at 36787.9 kD |
| Protein Sequence | | | |

MDSKYQCVKLNDGHFMPVLGFGTYAPAEVPKSKALEATKLAIEAGFRHIDSAHLYNNEEQVGLAIRSK
IADGSVKREDIFYTSKLWCNSHRPELVRPALERSLKNLQLDYVDLYLIHFPVSVKPGEEVIPKDENGK
ILFDTVDLCATWEAVEKCKDAGLAKSIGVSNFNRRQLEMILNKPGLKYKPVCNQVECHPYFNQRKLLD
FCKSKDIVLVAYSALGSHREEPWVDPNSPVLLEDPVLCALAKKHKRTPALIALRYQLQRGVVVLAKSY
NEQRIRQNVQVFEFQLTSEEMKAIDGLNRNVRYLTLDIFAGPPNYPFSDEY

A ClustalW comparison of the above protein sequences yields the following sequence alignment shown in Table 19B.

TABLE 19B
Comparison of the NOV19 protein sequences.

| | |
|---|---|
| NOV19a | -----------MDSKYQCVKLNDGHFMPVLGFGTYAPAEVPKSKALEATKLAIEAGFRHI |
| NOV19b | ----------------MMVTSCLSWDLAPMRLQRLLP-QVPKSKALEATKLAIEAGFRHI |
| NOV19c | -----------MDSKYQCVKLNDGHFMPVLGFGTYAPAEVPKSKALEATKLAIEAGFRHI |
| NOV19d | -----------MDSKYQCVKLNDGHFMPVLGFGTYAPAEVPKSKALEATKLAIEAGFRHI |
| NOV19e | -----------MDSKYQCVKLNDGHFMPVLGFGTYAPAEVPKSKALEAVKLAIEAGFHHI |
| NOV19f | --------TMAMDSKYQCVKLNDGHFMPVLGFGTYAPAEVPKSKALEATKLAIEAGFRHI |
| NOV19g | AGTTMGHHHHHHDSKYQCVKLNDGHFMPVLGFGTYAPAEVPKSKALEATKLAIEAGFRHI |
| NOV19h | -----------MDSKYQCVKLNDGHFMPVLGFGTYAPAEVPKSKALEAVKLAIEAGFHHI |
| NOV19i | -----------MDSKYQCVKLNDGHFMPVLGFGTYAPAEVPKSKALEATKLAIEAGFRHI |
| NOV19a | DSAHLYNNEEQVGLAIRSKIADGSVKREDIFYTSKLWCNSHRPELVRPALERSLKNLQLD |
| NOV19b | DSAHLYNNEEQVGLAIRSKIADGSVKREDIFYTSKLWCNSHRPELVRPALERSLKNLQLD |
| NOV19c | DSAHLYNNEEQVGLAIRSKIADGSVKREDIFYTSKLWCNSHRPELVRPALERSLKNLQLD |
| NOV19d | DSAHLYNNEEQVGLAIRSKIADGSVKREDIFYTSKLWCNSHRPELVRPALERSLKNLQLD |
| NOV19e | DSAHVYNNEEQVGLAIRSKIADGSVKREDIFYTSKLWSNSHRPELVRPALERSLKNLQLD |
| NOV19f | DSAHLYNNEEQVGLAIRSKIADGSVKREDIFYTSKLWCNSHRPELVRPALERSLKNLQLD |
| NOV19g | DSAHLYNNEEQVGLAIRSKIADGSVKREDIFYTSKLWCNSHRPELVRPALERSLKNLQLD |
| NOV19h | DSAHVYNNEEQVGLAIRSKIADGSVKREDIFYTSKLWSNSHRPELVRPALERSLKNLQLD |
| NOV19i | DSAHLYNNEEQVGLAIRSKIADGSVKREDIFYTSKLWCNSHRPELVRPALERSLKNLQLD |
| NOV19a | YVDLYLIHFPVSVKPGEEVIPKDENGKILFDTVDLCATWEAVEKCKDAGLAKSIGVSNFN |
| NOV19b | YVDLYLIHFPVSVKPGEEVIPKDESGKILFDTVDLCATWEAVEKCKDAGLAKSIGVSNFN |
| NOV19c | YVDLYLIHFPVSVKPGEEVIPKDENGKILFDTVDLCATWEAVEKCKDAGLAKSIGVSNFN |
| NOV19d | YVDLYLIHFPVSVKPGEEVIPKDENGKILFDTVDLCATWEAVEKCKDAGLAKSIGVSNFN |
| NOV19e | YVDLYLIHFPVSVKPGEEVIPKDENGKILFDTVDLCATWEAMEKCKDAGLAKSIGVSNFN |
| NOV19f | YVDLYLIHFPVSVKPGEEVIPKDENGKILFDTVDLCATWEAVEKCKDAGLAKSIGVSNFN |
| NOV19g | YVDLYLIHFPVSVKPGEEVIPKDENGKILFDTVDLCATWEAVEKCKDAGLAKSIGVSNFN |
| NOV19h | YVDLYLIHFPVSVKPGEEVIPKDENGKILFDTVDLCATWEAMEKCKDAGLAKSIGVSNFN |
| NOV19i | YVDLYLIHFPVSVKPGEEVIPKDENGKILFDTVDLCATWEAVEKCKDAGLAKSIGVSNFN |
| NOV19a | RRQLEMILNKPGLKYKPVCNQVECHPYFNQRKLLDFCKSKDIVLVAYSALGSHREEPWVD |
| NOV19b | RRQLEMILNKPGLKYKPVCNQVECHPYFNQRKLLDFCKSKDIVLVAYSALGSHREEPWVD |
| NOV19c | RRQLEMILNKPGLKYKPVCNQVECHPYFNQRKLLDFCKSKDIVLVAYSALGSHREEPWVD |
| NOV19d | RRQLEMILNKPGLKYKPVCNQVECHPYFNQRKLLDFCKSKDIVLVAYSALGSHREEPWVD |
| NOV19e | HRLLEMILNKPGLKYKPVCNQVECHPYFNQRKLLDFCKSKDIVLVAYSALGSHREEPWVD |
| NOV19f | RRQLEMILNKPGLKYKPVCNQVECHPYFNQRKLLDFCKSKDIVLVAYSALGSHREEPWVD |
| NOV19g | RRQLEMILNKPGLKYKPVCNQVECHPYFNQRKLLDFCKSKDIVLVAYSALGSHREEPWVD |
| NOV19h | HRLLEMILNKPGLKYKPVCNQVECHPYFNQRKLLDFCKSKDIVLVAYSALGSHREEPWVD |
| NOV19i | RRQLEMILNKPGLKYKPVCNQVECHPYFNQRKLLDFCKSKDIVLVAYSALGSHREEPWVD |
| NOV19a | PNSPVLLEDPVLCALAKKHKRTPALIALRYQLQRGVVVLAKSYNEQRIRQNVQVFEFQLT |
| NOV19b | PNSPVLLEDPVLCALAKKHKRTPALVALRYQLQRGVVVLAKSYNEQRIRQNVQVFEFQLT |
| NOV19c | PNSPVLLEDPVLCALAKKHKRTPALIALRYQLQRGVVVLAKRYNEQRIRQNVQVFEFQLT |
| NOV19d | PNSPVLLEDPVLCALAKKHKRTPALIALRYQLQRGVVVLAKSYNEQRIRQNVQVFEFQLT |
| NOV19e | PNSPVLLEDPVLCALAKKHKRTPALIALRYQLQRGVVVLAKSYNEQRIRQNVQVFEFQLT |
| NOV19f | PNSPVLLEDPVLCALAKKHKRTPALIALRYQLQRGVVVLAKSYNEQRIRQNVQVFEFQLT |
| NOV19g | PNSPVLLEDPVLCALAKKHKRTPALIALRYQLQRGVVVLAKSYNEQRIRQNVQVFEFQLT |
| NOV19h | PNSPVLLEDPVLCALAKKHKRTPALIALRYQLQRGVVVLAKSYNEQRIRQNVQVFEFQLT |
| NOV19i | PNSPVLLEDPVLCALAKKHKRTPALIALRYQLQRGVVVLAKSYNEQRIRQNVQVFEFQLT |
| NOV19a | SEEMKAIDGLNRNVRYLTLDIFAGPPNYPFSDEY--- |
| NOV19b | SEEMKAIDGLNRNVRYLTLDIFAGPPNYPFSDEY--- |
| NOV19c | SEEMKAIDGLNRNVRYLTLDIFAGPPNYPFSDEY--- |
| NOV19d | SEEMKAIDGLNRNVRYLTLDIFAGPPNYPFSD----- |
| NOV19e | SEEMKAIDGLNRNVRYLTLDIFAGPPNYPFSDEY--- |
| NOV19f | SEEMKAIDGLNRNVRYLTLDIFAGPPNYPFSD----- |
| NOV19g | SEEMKAIDGLNRNVRYLTLDIFAGPPNYPFSDEYLEG |
| NOV19h | SEEMKAIDGLNRNVRYLTLDIFAGPPNYPFSDEY--- |
| NOV19i | SEEMKAIDGLNRNVRYLTLDIFAGPPNYPFSDEY--- |
| NOV19a | (SEQ ID NO: 78) |
| NOV19b | (SEQ ID NO: 80) |
| NOV19c | (SEQ ID NO: 82) |
| NOV19d | (SEQ ID NO: 84) |
| NOV19e | (SEQ ID NO: 86) |
| NOV19f | (SEQ ID NO: 88) |
| NOV19g | (SEQ ID NO: 90) |
| NOV19h | (SEQ ID NO: 92) |
| NOV19i | (SEQ ID NO: 94) |

Further analysis of the NOV19a protein yielded the following properties shown in Table 19C.

TABLE 19C
Protein Sequence Properties NOV19a

| SignalP analysis: | No Known Signal Sequence Predicted | | | |
|---|---|---|---|---|
| PSORT II analysis: | PSG: a new signal peptide prediction method | | | |
| | N-region: length 9; pos. chg 2; neg. chg 1 | | | |
| | H-region: length 2; peak value −3.30 | | | |
| | PSG score: −7.70 | | | |
| | GvH: von Heijne's method for signal seq. recognition | | | |
| | GvH score (threshold: −2.1) : −5.32 | | | |
| | possible cleavage site: between 27 and 28 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = | 3.66 (at 253) | |
| | ALOM score: 3.66 (number of TMSs: 0) | | | |
| | MITDISC: discrimination of mitochondrial targeting seq | | | |
| | R content: | 0 | Hyd Moment (75): | 0.10 |
| | Hyd Moment | 4.27 | G content: | 0 |
| | (95): | | | |
| | D/E content: | 2 | S/T content: | 1 |

Score: −7.79
Gavel: prediction of cleavage sites for mitochondrial preseq
cleavage site motif not found
NUCDISC: discrimination of nuclearlocalization signals
pat4: KKHK (3) at 246
pat4: KHKR (3) at 247
pat7: none
bipartite: none
content of basic residues: 13.3%
NLS Score: −0.10
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern : none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 76.7
COIL: Lupas's algorithm to detect coiled-coil regions
total: 0 residues
Final Results (k = 9/23):
34.8%: cytoplasmic
30.4%: mitochondrial
30.4%: nuclear
4.3%: vacuolar
>> prediction for CG59693-01 is cyt (k = 23)

A search of the NOV19a protein against the Geneseq database, a proprietary database that contains sequences published in patents and patent publication, yielded several homologous proteins shown in Table 19D.

TABLE 19D
Geneseq Results for NOV19a

| | | NOV19a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB43444 | Human cancer associated protein | 1 . . . 323 | 318/323 (98%) | 0.0 |
| | sequence SEQ ID NO: 889 - Homo | 14 . . . 336 | 318/323 (98%) | |
| | sapiens, 336 aa. [WO200055350- | | | |
| | A1, 21 SEP. 2000] | | | |
| AAU85559 | Clone #59314 (L1426P) of lung | 1 . . . 323 | 311/323 (96%) | 0.0 |
| | tumour protein - Homo sapiens, | 1 . . . 323 | 316/323 (97%) | |
| | 323 aa. [WO200204514-A2, 17 | | | |
| | JAN. 2002] | | | |
| ABB75050 | Human lung tumour L773P | 29 . . . 323 | 288/295 (97%) | e−168 |
| | recombinant protein sequence | 77 . . . 371 | 290/295 (97%) | |
| | SEQ ID NO: 433 - Homo sapiens, | | | |
| | 371 aa. [WO200200174-A2, 03 | | | |
| | JAN. 2002] | | | |
| ABB74958 | Human lung tumour L773P | 29 . . . 323 | 288/295 (97%) | e−168 |
| | protein sequence SEQ ID NO: 172 - | 70 . . . 364 | 290/295 (97%) | |
| | Homo sapiens, 364 aa. | | | |
| | [WO200200174-A2, 03 JAN. | | | |
| | 2002] | | | |
| AAU85520 | L773P lung tumour protein - | 29 . . . 323 | 288/295 (97%) | e−168 |
| | Homo sapiens, 364 aa. | 70 . . . 364 | 290/295 (97%) | |
| | [WO200204514-A2, 17 JAN. | | | |
| | 2002] | | | |

In a BLAST search of public sequence databases, the NOV19a protein was found to have homology to the proteins shown in the BLASTP data in Table 19E.

TABLE 19E
Public BLASTP Results for NOV19a

| | | NOV19a | Identities/ | |
|---|---|---|---|---|
| Protein | | Residues/ | Similarities | |
| Accession | | Match | for the Matched | Expect |
| Number | Protein/Organism/Length | Residues | Portion | Value |
| Q04828 | Aldo-keto reductase family 1 | 1 . . . 323 | 323/323 (100%) | 0.0 |
| | member C1 (EC 1.1.1.-) (Trans-1,2- | 1 . . . 323 | 323/323 (100%) | |
| | dihydrobenzene-1,2-diol | | | |
| | dehydrogenase) (EC 1.3.1.20) (High- | | | |
| | affinity hepatic bile acid-binding | | | |
| | protein) (HBAB) (Chlordecone | | | |
| | reductase homolog HAKRC) | | | |
| | (Dihydrodiol dehydrogenase 2) | | | |
| | (DD2) (20 alpha- hydroxysteroid | | | |
| | dehydrogenase) - Homo sapiens | | | |
| | (Human), 323 aa. | | | |
| P52895 | Aldo-keto reductase family 1 | 1 . . . 323 | 316/323 (97%) | 0.0 |
| | member C2 (EC 1.1.1.-) (Trans-1,2- | 1 . . . 323 | 318/323 (97%) | |
| | dihydrobenzene-1,2-diol | | | |
| | dehydrogenase) (EC 1.3.1.20) | | | |
| | (Chlordecone reductase homolog | | | |
| | HAKRD) (Dihydrodiol | | | |
| | dehydrogenase/bile acid-binding | | | |
| | protein) (DD/BABP) (Dihydrodiol | | | |
| | dehydrogenase 2) (DD2) - Homo | | | |
| | sapiens (Human), 323 aa. | | | |
| I73676 | chlordecone reductase homolog | 1 . . . 323 | 313/323 (96%) | 0.0 |
| | (clone HAKRd) - human, 323 aa. | 1 . . . 323 | 317/323 (97%) | |
| I73675 | chlordecone reductase homolog | 4 . . . 323 | 312/320 (97%) | 0.0 |
| | (clone HAKRc) - human, 320 aa | 1 . . . 320 | 313/320 (97%) | |
| | (fragment). | | | |
| Q95JH6 | 3(20)alpha- | 1 . . . 323 | 304/323 (94%) | 0.0 |
| | hydroxysteroid/dihydrodiol/indanol | 1 . . . 323 | 319/323 (98%) | |
| | dehydrogenase (EC 1.1.1.112) - | | | |
| | Macaca fuscata (Japanese macaque), | | | |
| | 323 aa. | | | |

PFam analysis predicts that the NOV19a protein contains the domains shown in the Table 19F.

TABLE 19F
Domain Analysis of NOV19a

| | | Identities/ | |
|---|---|---|---|
| | | Similarities | |
| | NOV19a | for the Matched | Expect |
| Pfam Domain | Match Region | Region | Value |
| aldo_ket_red | 10 . . . 303 | 164/369 (44%) | 3.4e−156 |
| | | 274/369 (74%) | |

### Example 20

The NOV20 clone was analyzed, and the nucleotide and encoded polypeptide sequences are shown in Table 20A.

TABLE 20A
NOV20 Sequence Analysis

| | | |
|---|---|---|
| NOV20a, CG93088-01 | SEQ ID NO: 95 | 3815 bp |
| DNA Sequence | ORF Start: ATG at 263 | ORF Stop: end of sequence |

CGGCCGCGATCCCCACCACACCACCAGCCCGGCCGCACGGGGCACTGAGCCGGGTGCTGAGCACCGGA
GGCCCCGCCGAGGCCGGGACTCAGATGTTGAAAGTTAATTTGTGTAAAGACTTATGCACGTGGTGACA
TGAGTTCTGCCCAGTGCTCTGAAATCAAAGTGAAGAAATAAATCCATGGAAGCCCAGGCAAATGATGG
GTGTAGCTATGACTCTCTGAAGGACCTGCAGAGAAACGCCTCCTGATTTTGTCTTACAATGGAACTTA
AAAAGTCGCCTGACGGTGGATGGGGCTGGGTGATTGTGTTTGTCTCCTTCCTTACTCAGTTTTTGTGT
TACGGATCCCCACTAGCTGTTGGAGTCCTGTACATAGAATGGCTGGATGCCTTTGGTGAAGGAAAAGG
AAAAACAGCCTGGGTTGGATCCCTGGCAAGTGGAGTTGGCTTGCTTGCAAGTCCTGTCTGCAGTCTCT
GTGTCTCATCTTTTGGAGCAAGACCTGTCACAATCTTCAGTGGCTTCATGGTGGCTGGAGGCCTGATG
TTGAGCAGTTTTGCTCCCAATATCTACTTTCTGTTTTTTTCCTATGGCATTGTTGTAGGTCTTGGATG
TGGTTTATTATACACTGCAACAGTGACCATTACGTGCCAGTATTTTGACGATCGCCGAGGCCTAGCGC
TTGGCCTGATTTCAACAGGTTCAAGCGTTGGCCTTTTCATATATGCTGCTCTGCAGAGGATGCTGGTT
GAGTTCTATGGACTGGATGGATGCTTGCTGATTGTGGGTGCTTTAGCTTTAAATATATTAGCCTGTGG
CAGTCTGATGAGACCCCTCCAATCTTCTGATTGTCCTTTGCCTAAAAAAATAGCTCCAGAAGATCTAC
CAGATAAATACTCCATTTACAATGAAAAAGGAAAGAATCTGGAAGAAAACATAAACATTCTTGACAAG
AGCTACAGTAGTGAGGAAAAATGCAGGATCACGTTAGCCAATGGTGACTGGAAACAAGACAGCCTACT
TCATAAAAACCCCACAGTGACACACACAAAAGAGCCTGAAACGTACAAAAAGAAAGTTGCAGAACAGA
CATATTTTTGCAAACAGCTTGCCAAGAGGAAGTGGCAGTTATATAAAAACTACTGTGGTGAAACTGTG
GCTCTTTTTAAAAACAAAGTATTTTCAGCCCTTTTCATTGCTATCTTACTCTTTGACATCGGAGGGTT
TCCACCTTCATTACTTATGGAAGATGTAGCAAGAAGTTCAAACGTGAAAGAAGAAGAGTTTATTATGC
CACTTATTTCCATTATAGGCATTATGACAGCAGTTGGTAAACTGCTTTTAGGGATACTGGCTGACTTC
AAGTGGATTAATACCTTGTATCTTTATGTTGCTACCTTAATCATCATGGGCCTAGCCTTGTGTGCAAT
TCCATTTGCCAAAAGCTATGTCACATTGGCGTTGCTTTCTGGGATCCTAGGGTTTCTTACTGGTAATT
GGTCCATCTTTCCATATGTGACCACGAAGACTGTGGGAATTGAAAAATTAGCCCATGCCTATGGGATA
TTAATGTTCTTTGCTGGACTTGGAAATAGCCTAGGACCACCATCGTTGGGTTGGTTTTATGACTGGAC
CCAGACCTATGATATTGCATTTTATTTTAGTGGCTTCTGCGTCCTGCTGGGAGGTTTTATTCTGCTGC
TGGCAGCCTTGCCCTCTTGGGATACATGCAACAAGCAACTCCCCAAGCCAGCTCCAACAACTTTCTTG
TACAAAGTTGCCTCTAATGTTTAGAAGAATATTGGAAGACACTATTTTTGCTATTTTATACCATATAG
CAACGATATTTTAACAGATTCTCAAGCAAATTTTCTAGAGTCAAGACTATTTTCTCATAGCAAAATTT
CACAATGACTGACTCTGAATGAATTATTTTTTTTTATATATCCTATTTTTTATGTAGTGTATGCGTAG
CCTCTATCTCGTATTTTTTTCTATTTCTCCTCCCCACACCATCAATGGGACTATTCTGTTTTGCTGTT
ATTCACTAGTTCTTAACATTGTAAAAAGTTTGACCAGCCTCAGAAGGCTTTCTCTGTGTAAAGAAGTA
TAATTTCTCTGCTGACTCCATTTAATCCACTGCAAGGCACCTAGAGAGACTGCTCCTATTTTAAAAGT
GATGCAAGCATCATGATAAGATATGTGTGAAGCCCACTAGGAAATAAATCATTCTCTTCTCTATGTTT
GACTTGCTAGTAAACAGAAGACTTCAAGCCAGCCAGGAAATTAAAGTGGCGACTAAAACAGCCTTAAG
AATTGCAGTGGAGCAAATTGGTCATTTTTTAAAAAAATATATTTTAACCTACAGTCACCAGTTTTCAT
TATTCTATTTACCTCACTGAAGTACTCGCATGTTGTTTGGTACCCACTGAGCAACTGTTTCAGTTCCT
AAGGTATTTGCTGAGATGTGGGTGAACTCCAAATGGAGAAGTAGTCACTGTAGACTTTCTTCATGGTT
GACCACTCCAACCTTGCTCACTTTTGCTTCTTGGCCATCCACTCAGCTGATGTTTCCTGGAAGTGCTA
ATTTTACCTGTTTCCAAATTGGAAACACATTTCTCAATCATTCCGTTCTGGCAAATGGGAAACATCCA
TTTGCTTTGGGCACAGTGGGGATGGGCTGCAAGTTCTTGCATATCCTCCCAGTGAAGCATTTATTTGC
TACTATCAGATTTTACCACTATCAAATATAATTCAAGGGCAGAATTAAACGTGAGTGTGTGTGTGTGT
GTGTGTGTGTGTGCTATGCATGCTCTAAGTCTGCATGGGATATGGGAATGGAAAAGGGCAATAAGAAA
TTAATACCCTTATGCAGTTGCATTTAACCTTAAGAAAAATGTCCTTGGGATAAACTCCAATGTTTAAT
ACATTGATTTTTTTTCTAAAGAAATGGGTTTTAAACTTTGGTATGCATCAGAATTCCCTATAGATCTT
TTTGAAAATATAGGTACCTGGGTATCACACATAGAACTTTTAATTCTGCTGGTGTAGGCTGTTGCCCA
AACATCTATAATTTTACTGAGCTCTTCAAGTGATTCTGATAACACAGCCTGGATTGAGAATTTTTATA
AGATTGGCAATGGAAAAACATTTATTCTTTTAAATAATAATTTTTTTAAAACCCAAGAGGTCAGGGGA
TTTTATAAACCAATAGCCAAGTGTTCTTTAAATAGGAGGCACCCTTCCCATTGTGCCAAAATCATCTT
TTCATTTATTTTGAAATTTGTATGATTATTTTATACTTGTATGTTGCCTTTCTTCGAAGGCGCCTGAA
GCACTTTATAAACACAAATCCTCACAATACCTCTGTGAGGTAGGTAAATAGTACTTTTCTATGTAGTA
AACCTGGAATATGGAGAATTTCATAACAGTTCATTCTACTTAATAATGCAATAATGGAGCTCCAAGTT
GTCTTGGACTTCTACACCACACTCAGACTTCTGGAAAGTTTTCTGTACCTCATTCTTTAGTCCCTGTC
AAGGTTAGTAAATAAAATAAGTGACATAAAAAAAAAAAAAAAACTAAACTACTTGTTGTGTTGAAAGT
TCCTTTTTGCCAGTTATGTTCAGGAAACCCAATAACCTGAAAAAGTTTGACTTTGATGTGACATCTTC
ATATTCATCAATGCTGATAATTGTCCAAAGGCATCTTCACTATGTCTGCTAAATAACATCCAATGTGG
GCGTTATCTGTTGTCTAGGGGATGAATTTTAAGTTACAATAAAATATTTTTCTTTGTTTTGCATCAAA
AAAAAAA

| | | | |
|---|---|---|---|
| NOV20a, CG93088-01 | SEQ ID NO: 96 | 509 aa | MW at 55780.8 kD |
| Protein Sequence | | | |

MELKKSPDGGWGWVIVFVSFLTQFLCYGSPLAVGVLYIEWLDAFGEGKGKTAWVGSLASGVGLLASPV
CSLCVSSFGARPVTIFSGFMVAGGLMLSSFAPNIYFLFFSYGIVVGLGCGLLYTATVTITCQYFDDRR
GLALGLISTGSSVGLFIYAALQRMLVEFYGLDGCLLIVGALALNILACGSLMRPLQSSDCPLPKKIAP
EDLPDKYSIYNEKGKNLEENINILDKSYSSEEKCRITLANGDWKQDSLLHKNPTVTHTKEPETYKKKV
AEQTYFCKQLAKRKWQLYKNYCGETVALFKNKVFSALFIAILLFDIGGFPPSLLMEDVARSSNVKEEE
FIMPLISIIGIMTAVGKLLLGILADFKWINTLYLYVATLIIMGLALCAIPFAKSYVTLALLSGILGFL
TGNWSIFPYVTTKTVGIEKLAHAYGILMFFAGLGNSLGPPSLGWFYDWTQTYDIAFYFSGFCVLLGGF
ILLLAALPSWDTCNKQLPKPAPTTFLYKVASNV

Further analysis of the NOV20a protein yielded the following properties shown in Table 20B.

TABLE 20B
Protein Sequence Properties NOV20a

| SignalP analysis: | Cleavage site between residues 29 and 30 | | | | |
|---|---|---|---|---|---|
| PSORT II analysis: | PSG: a new signal peptide prediction method | | | | |
| | N-region: length 8; pos. chg 2; neg. chg 2 | | | | |
| | H-region: length 30; peak value 11.36 | | | | |
| | PSG score: 6.96 | | | | |
| | GvH: von Heijne's method for signal seq. recognition | | | | |
| | GvH score (threshold: −2.1) : −3.41 | | | | |
| | possible cleavage site: between 28 and 29 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 11 | | | | |
| | INTEGRAL | Likelihood = | −0.59 | Transmembrane | 10-26 |
| | INTEGRAL | Likelihood = | −2.50 | Transmembrane | 57-73 |
| | INTEGRAL | Likelihood = | −0.85 | Transmembrane | 83-99 |
| | INTEGRAL | Likelihood = | −4.46 | Transmembrane | 104-120 |
| | INTEGRAL | Likelihood = | −0.75 | Transmembrane | 137-153 |
| | INTEGRAL | Likelihood = | −6.32 | Transmembrane | 171-187 |
| | INTEGRAL | Likelihood = | −6.32 | Transmembrane | 305-321 |
| | INTEGRAL | Likelihood = | −5.41 | Transmembrane | 348-364 |
| | INTEGRAL | Likelihood = | −7.38 | Transmembrane | 376-392 |
| | INTEGRAL | Likelihood = | 0.05 | Transmembrane | 394-410 |
| | INTEGRAL | Likelihood = | −7.59 | Transmembrane | 467-483 |
| | PERIPHERAL | Likelihood = | 3.13 (at 423) | | |
| | ALOM score: −7.59 (number of TMSs: 11) | | | | |
| | MTOP: Prediction of membrane topology (Hartmann et al.) | | | | |
| | Center position for calculation: 17 | | | | |
| | Charge difference: −2.0 C(−1.0) - N( 1.0) | | | | |
| | N >= C: N-terminal side will be inside | | | | |
| | >>> membrane topology: type 3a | | | | |
| | MITDISC: discrimination of mitochondrial targeting seq | | | | |
| | R content: | 0 | Hyd Moment (75): | 5.89 | |
| | Hyd Moment (95): | 8.05 | G content: | 0 | |
| | D/E content: | 2 | S/T content: | 1 | |
| | Score: −6.55 | | | | |
| | Gavel: prediction of cleavage sites for mitochondrial preseq | | | | |
| | cleavage site motif not found | | | | |
| | NUCDISC: discrimination of nuclear localization signals | | | | |
| | pat4: none | | | | |
| | pat7: PETYKKK (3) at 265 | | | | |
| | bipartite: KKVAEQTYFCKQLAKRK at 270 | | | | |
| | content of basic residues: 7.9% | | | | |
| | NLS Score: 0.28 | | | | |
| | KDEL: ER retention motif in the C-terminus: none | | | | |
| | ER Membrane Retention Signals: none | | | | |
| | SKL: peroxisomal targeting signal in the C-terminus: none | | | | |
| | PTS2: 2nd peroxisomal targeting signal: none | | | | |
| | VAC: possible vacuolar targeting motif: none | | | | |
| | RNA-binding motif: none | | | | |
| | Actinin-type actin-binding motif: | | | | |
| | type 1: none | | | | |
| | type 2: none | | | | |
| | NMYR: N-myristoylation pattern: none | | | | |
| | Prenylation motif: none | | | | |
| | memYQRL: transport motif from cell surface to Golgi: none | | | | |
| | Tyrosines in the tail: none | | | | |
| | Dileucine motif in the tail: none | | | | |
| | checking 63 PROSITE DNA binding motifs: none | | | | |
| | checking 71 PROSITE ribosomal protein motifs: none | | | | |
| | checking 33 PROSITE prokaryotic DNA binding motifs: none | | | | |
| | NNCN: Reinhardt's method for Cytoplasmic/Nuclear discrimination | | | | |
| | Prediction: cytoplasmic | | | | |
| | Reliability: 94.1 | | | | |
| | COIL: Lupas's algorithm to detect coiled-coil regions | | | | |
| | 208 D | 0.71 | | | |
| | 209 K | 0.75 | | | |
| | 210 Y | 0.75 | | | |
| | 211 S | 0.75 | | | |
| | 212 I | 0.75 | | | |
| | 213 Y | 0.75 | | | |
| | 214 N | 0.75 | | | |
| | 215 E | 0.75 | | | |
| | 216 K | 0.75 | | | |
| | 217 G | 0.75 | | | |
| | 218 K | 0.75 | | | |
| | 219 N | 0.75 | | | |
| | 220 L | 0.75 | | | |
| | 221 E | 0.75 | | | |
| | 222 E | 0.75 | | | |
| | 223 N | 0.75 | | | |
| | 224 I | 0.75 | | | |
| | 225 N | 0.75 | | | |
| | 226 I | 0.75 | | | |
| | 227 L | 0.75 | | | |
| | 228 D | 0.75 | | | |
| | 229 K | 0.75 | | | |
| | 230 S | 0.75 | | | |
| | 231 Y | 0.75 | | | |
| | 232 S | 0.75 | | | |
| | 233 S | 0.75 | | | |
| | 234 E | 0.75 | | | |
| | 235 E | 0.75 | | | |
| | 236 K | 0.75 | | | |

total: 29 residues
Final Results (k = 9/23) :
66.7%: endoplasmic reticulum
22.2%: mitochondrial
11.1%: vesicles of secretory system
>> prediction for CG93088-01 is end (k = 9)

A search of the NOV20a protein against the Geneseq database, a proprietary database that contains sequences published in patents and patent publication, yielded several homologous proteins shown in Table 20C.

TABLE 20C
Geneseq Results for NOV20a

| | | NOV20a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABU12075 | Human NOV19a CG93088-01 | 1 . . . 509 | 509/509 (100%) | 0.0 |
| | protein SEQ ID 70 - Homo | 1 . . . 509 | 509/509 (100%) | |
| | sapiens, 509 aa. [WO200281625- | | | |
| | A2, 17 OCT. 2002] | | | |
| ABG61543 | Human transporter and ion | 1 . . . 509 | 507/509 (99%) | 0.0 |
| | channel, TRICH13, Incyte ID | 1 . . . 509 | 508/509 (99%) | |
| | 4027693CD1 - Homo sapiens, | | | |
| | 509 aa. [WO200240541-A2, 23 | | | |
| | MAY 2002] | | | |
| ABB83901 | Human transporter protein SEQ | 1 . . . 509 | 507/509 (99%) | 0.0 |
| | ID NO 2 - Homo sapiens, 509 aa. | 1 . . . 509 | 508/509 (99%) | |
| | [WO200257310-A2, 25 JUL. | | | |
| | 2002] | | | |
| ABP51421 | Human MDDT SEQ ID NO 443 - | 3 . . . 507 | 168/509(33%) | 3e−74 |
| | Homo sapiens, 529 aa. | 38 . . . 527 | 265/509 (52%) | |
| | [WO200240715-A2, 23 MAY | | | |
| | 2002] | | | |
| ABG33043 | Human 25466 transporter | 3 . . . 480 | 164/482 (34%) | 3e−74 |
| | protein - Homo sapiens, 510 aa. | 22 . . . 490 | 256/482 (53%) | |
| | [EP1233024-A2, 21 AUG. 2002] | | | |

In a BLAST search of public sequence databases, the NOV20a protein was found to have homology to the proteins shown in the BLASTP data in Table 20D.

TABLE 20D
Public BLASTP Results for NOV20a

| | | NOV20a | Identities/ | |
|---|---|---|---|---|
| Protein | | Residues/ | Similarities for | |
| Accession | | Match | the Matched | Expect |
| Number | Protein/Organism/Length | Residues | Portion | Value |
| BAB23369 | Adult male lung cDNA, RIKEN | 1 . . . 509 | 445/511 (87%) | 0.0 |
| | full-length enriched library, | 1 . . . 508 | 471/511 (92%) | |
| | clone:1200003C15 | | | |
| | product:hypothetical protein, full | | | |
| | insert sequence - Mus musculus | | | |
| | (Mouse), 508 aa. | | | |
| Q9UFH8 | Hypothetical protein - Homo | 353 . . . 509 | 155/157 (98%) | 3e−87 |
| | sapiens (Human), 157 aa | 1 . . . 157 | 156/157 (98%) | |
| | (fragment). | | | |
| Q9CPZ7 | 4930425B13Rik protein | 352 . . . 509 | 148/159 (93%) | 2e−82 |
| | (1200003C15Rik protein) - Mus | 1 . . . 159 | 152/159 (95%) | |
| | musculus (Mouse), 159 aa. | | | |
| CAD52855 | Sequence 1 from Patent | 3 . . . 480 | 164/482 (34%) | 1e−73 |
| | EP1233024 - Homo sapiens | 22 . . . 490 | 256/482 (53%) | |
| | (Human), 510aa. | | | |
| AAH47967 | Similar to solute carrier family 16 | 5 . . . 509 | 140/509 (27%) | 4e−50 |
| | (monocarboxylic acid | 22 . . . 499 | 234/509 (45%) | |
| | transporters), member 6 - Xenopus | | | |
| | laevis (African clawed frog), 533 | | | |
| | aa. | | | |

PFam analysis predicts that the NOV20a protein contains the domains shown in the Table 20E.

TABLE 20E
Domain Analysis of NOV20a

| | | Identities/ | |
|---|---|---|---|
| | NOV20a | Similarities for | |
| Pfam | Match | the Matched | Expect |
| Domain | Region | Region | Value |
| sugar_tr | 11 . . . 456 | 72/548 (13%) | 0.17 |
| | | 278/548 (51%) | |

### Example B

### Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth, factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Ga14-activation domain (Ga14-AD) fusion). Such cDNA libraries as well as commercially available cDNA libraries from Clontech (Palo Alto, Calif.) were then transferred from E.coli into a CuraGen Corporation proprietary yeast strain (disclosed in U.S. Pat. Nos. 6,057,101 and 6,083,693, incorporated herein by reference in their entireties).

Ga14-binding domain (Ga14-BD) fusions of a CuraGen Corportion proprietary library of human sequences was used to screen multiple Ga14-AD fusion cDNA libraries resulting in the selection of yeast hybrid diploids in each of which the Ga14-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma-Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/SI (containing human tissues and cell lines with an emphasis on metabolic diseases), AI_comprehensive_panel (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. Expression with CT values below 28 is considered as high expression, CT values between 28 and 32 is considered moderate and CT value between 32 to 35 is considered as low expression. All the relative expression with CT values above 35 is not considered as significant expression.

When working with sscDNA samples, normalized sscDNA was used as described previously for RNA samples. PCR reactions containing one or two sets of probe and primers were set up as described previously, using 1×TaqMang. Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff p1 effusion=pleural effusion,

glio=glioma,

astro=astrocytoma, and

neuro=neuroblastoma.

General_screening_panel_v1.4, v1.5, v1.6 and 1.7

The plates for Panels 1.4, 1.5, 1.6 and 1.7 include 2 control wells (genomic DNA control and chemistry control) and 88 to 94 wells containing cDNA from various samples. The samples in Panels 1.4, 1.5, 1.6 and 1.7 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, 1.5, 1.6 and 1.7 are widely available through the American Type Culture Collection (ATCC), a repository for cultured cell lines, and were cultured using the conditions recommended by the ATCC. The normal tissues found on Panels 1.4, 1.5, 1.6 and 1.7 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D, 2.2, 2.3 and 2.4

The plates for Panels 2D, 2.2, 2.3 and 2.4 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

HASS Panel v 1.0

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

ARDAIS Panel v 1.0

The plates for ARDAIS panel v 1.0 generally include 2 control wells and 22 test samples composed of RNA isolated from human tissue procured by surgeons working in close cooperation with Ardais Corporation. The tissues are derived from human lung malignancies (lung adenocarcinoma or lung squamous cell carcinoma) and in cases where indicated many malignant samples have “matched margins” obtained from noncancerous lung tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). Unmatched malignant and non-malignant RNA samples from lungs were also obtained from Ardais. Additional information from Ardais provides a gross histopathological assessment of tumor differentiation grade and stage. Moreover, most samples include the original surgical pathology report that provides information regarding the clinical state of the patient.

ARDAIS Prostate v 1.0

The plates for ARDAIS prostate 1.0 generally include 2 control wells and 68 test samples composed of RNA isolated from human tissue procured by surgeons working in close cooperation with Ardais Corporation. The tissues are derived from human prostate malignancies and in cases where indicated malignant samples have “matched margins” obtained from noncancerous prostate tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). RNA from unmatched malignant and non-malignant prostate samples were also obtained from Ardais. Additional information from Ardais provides a gross histopathological assessment of tumor differentiation grade and stage. Moreover, most samples include the original surgical pathology report that provides information regarding the clinical state of the patient.

Panel 3D, 3.1 and 3.2

The plates of Panel 3D, 3.1, and 3.2 are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D, 3.1, 3.2, 1, 1.1., 1.2, 1.3D, 1.4, 1.5, and 1.6 are of the most common cell lines used in the scientific literature.

Panels 4D, 4R, and 4.1D

Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 1 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

AI_comprehensive panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS)(BA)(MF)=Individual patients

Adj=Adjacent tissue

Match control=adjacent tissues

−M=Male

−F=Female

COPD=Chronic obstructive pulmonary disease

AI.05 Chondrosarcoma

The AI.05 chondrosarcoma plates are comprised of SW1353 cells that had been subjected to serum starvation and treatment with cytokines that are known to induce MMP (1, 3 and 13) synthesis (eg. IL1beta). These treatments include: IL-1beta (10 ng/ml), IL-1beta+TNF-alpha (50 ng/ml), IL-1beta+Oncostatin (50 ng/ml) and PMA (100 ng/ml). The SW1353 cells were obtained from the ATCC (American Type Culture Collection) and were all cultured under standard recommended conditions. The SW1353 cells were plated at 3×10⁵ cells/ml (in DMEM medium-10% FBS) in 6-well plates. The treatment was done in triplicate, for 6 and 18 h. The supernatants were collected for analysis of MMP 1, 3 and 13 production and for RNA extraction. RNA was prepared from these samples using the standard procedures.

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (less than 1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adiocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT Uterus

PL=Placenta

AD=Adipose Differentiated

AM Adipose Midway Differentiated

U=Undifferentiated Stem Cells

Human Metabolic RTQ-PCR Panel

The plates for the Human Metabolic RTQ-PCR Panel include two control wells (genomic DNA control and chemistry control) and 211 cDNAs isolated from human tissues and cell lines with an emphasis on metabolic diseases. This panel is useful for establishing the tissue and cellular expression profiles for genes believed to play a role in the etiology and pathogenesis of obesity and/or diabetes and to confirm differential expression of such genes derived from other methods. Metabolic tissues were obtained from patients enrolled in the CuraGen Gestational Diabetes study and from autopsy tissues from Type II diabetics and age, sex and race-matched control patients. One or more of the following were used to characterize the patients: body mass index [BMI=wt(kg)/ht(m²)], serum glucose, HgbA1c. Cell lines used in this panel are widely available through the American Type Culture Collection (ATCC), a repository for cultured cell lines. RNA from human Pancreatic Islets was also obtained.

In the Gestational Diabetes study, subjects are young (18-40 years), otherwise healthy women with and without gestational diabetes undergoing routine (elective) Caesarian section. After delivery of the infant, when the surgical incisions were being repaired/closed, the obstetrician removed a small sample (less than 1 cc) of the exposed metabolic tissues during the closure of each surgical level. The biopsy material was rinsed in sterile saline, blotted, and then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus), and subcutaneous adipose. Patient descriptions are as follows:

Patient 7—Non-diabetic Caucasian and obese

Patient 8—Non-diabetic Caucasian and obese

Patient 12—Diabetic Caucasian with unknown BMI and on insulin

Patient 13—Diabetic Caucasian, overweight, not on insulin

Patient 15—Diabetic Caucasian, obese, not on insulin

Patient 17—Diabetic Caucasian, normal weight, not on insulin

Patient 18—Diabetic Hispanic, obese, not on insulin

Patient 19—Non-diabetic Caucasian and normal weight

Patient 20—Diabetic Caucasian, overweight, and on insulin

Patient 21—Non-diabetic Caucasian and overweight

Patient 22—Diabetic Caucasian, normal weight, on insulin

Patient 23—Non-diabetic Caucasian and overweight

Patient 25—Diabetic Caucasian, normal weight, not on insulin

Patient 26—Diabetic Caucasian, obese, on insulin

Patient 27—Diabetic Caucasian, obese, on insulin

Total RNA was isolated from metabolic tissues of 12 Type II diabetic patients and 12 matched control patients included hypothalamus, liver, pancreas, small intestine, psoas muscle, diaphragm muscle, visceral adipose, and subcutaneous adipose. The diabetics and non-diabetics were matched for age, sex, ethnicity, and BMI where possible.

The panel also contains pancreatic islets from a 22 year old male patient (with a BMI of 35) obtained from the Diabetes Research Institute at the University of Miami School of Medicine. Islet tissue was processed to total RNA at CuraGen.

Cell lines used in this panel are widely available through the American Type Culture Collection (ATCC), a repository for cultured cell lines, and were cultured at an outside facility. The RNA was extracted at CuraGen according to CuraGen protocols. All samples were then processed at CuraGen to produce single stranded cDNA.

In the labels used to identify tissues in the Human Metabolic panel, the following abbreviations are used:

Pl=placenta

Go=greater omentum

Sk=skeletal muscle

Ut=uterus

CC=Caucasian

HI=Hispanic

A=African American

AS=Asian

Diab=Type II diabetic

Norm=Non-diabetic

Overwt=Overweight; med BMI

Obese=Hi BMI

Low BM=20-25

Med BM=26-30

Hi BMI=Greater than 30

M=Male

#=Patient identifier

Vis.=Visceral

SubQ=Subcutaneous

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supemuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimeres like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

Panel CNS_Neurodegeneration_V2.0

The plates for Panel CNS_Neurodegeneration_V2.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains sixteen brains from Alzheimer's disease (AD) patients, and twenty-nine brains from “Normal controls” who showed no evidence of dementia prior to death. The twenty-nine normal control brains are divided into two categories: Fourteen controls with no dementia and no Alzheimer's like pathology (Controls) and fifteen controls with no dementia but evidence of severe Alzheimer's like pathology, (specifically senile plaque load rated as level 3 on a scale of 0-3; 0=no evidence of plaques, 3=severe AD senile plaque load). Tissue from the temporal cotex (Broddmann Area 21) was selected for all samples from the Harvard Brain Tissue Resource Center; from the two sample from the Human Brain and Spinal Fluid Resource Center (samples 1 and 2) tissue from the inferior and superior temporal cortex was used; each sample on the panel represents a pool of inferior and superior temporal cortex from an individual patient. The temporal cortex was chosen as it shows a loss of neurons in the intermediate stages of the disease. Selection of a region which is affected in the early stages of Alzheimer's disease (e.g., hippocampus or entorhinal cortex) could potentially result in the examination of gene expression after vulnerable neurons are lost, and missing genes involved in the actual neurodegeneration process.

In the labels employed to identify tissues in the CNS_Neurodegeneration_V2.0 panel, the following abbreviations are used:

AD=Alzheimer's disease brain; patient was demented and showed AD-like pathology upon autopsy

Control=Control brains; patient not demented, showing no neuropathology

AH3=Control brains; pateint not demented but showing sever AD-like pathology

Inf & Sup Temp Ctx Pool=Pool of inferior and superior temporal cortex for a given individual

A. CG164221-01: Protein kinase, Sgk

Expression of gene CG164221-01 was assessed using the primer-probe set Ag6086, described in Table AA. Results of the RTQ-PCR runs are shown in Tables AB, AC and AD.

Table AA. Probe Name Ag6086

TABLE AA

Probe Name Ag6086

| | | | | |
|---|---|---|---|---|
| | | | Start | SEQ ID |
| Primers | Sequences | Length | Position | No |

| | | | | |
|---|---|---|---|---|
| Forward | 5′-aattctcatcgctttcatgaag-3′ | 22 | 101 | 98 |
| Probe | TET-5′-aagtcgttcagacccatcctcctctg-3′-TAMRA | 26 | 123 | 99 |
| Reverse | 5′-ataggagttattggcaatcttctga-3′ | 25 | 152 | 100 |

Table AB. CNS neurodegeneration_v1.0

TABLE AB
CNS_neurodegeneration_v1.0

| Tissue Name | A | B |
|---|---|---|
| AD 1 Hippo | 14.0 | 13.8 |
| AD 2 Hippo | 26.4 | 17.6 |
| AD 3 Hippo | 6.0 | 5.9 |
| AD 4 Hippo | 6.2 | 5.0 |
| AD 5 Hippo | 26.4 | 25.5 |
| AD 6 Hippo | 67.8 | 58.2 |
| Control 2 Hippo | 20.2 | 19.1 |
| Control 4 Hippo | 12.9 | 12.2 |
| Control (Path) 3 Hippo | 3.1 | 4.5 |
| AD 1 Temporal Ctx | 22.4 | 25.9 |
| AD 2 Temporal Ctx | 25.9 | 22.5 |
| AD 3 Temporal Ctx | 3.1 | 3.3 |
| AD 4 Temporal Ctx | 18.3 | 17.2 |
| AD 5 Inf Temporal Ctx | 40.1 | 72.2 |
| AD 5 Sup Temporal Ctx | 47.6 | 39.8 |
| AD 6 Inf Temporal Ctx | 100.0 | 100.0 |
| AD 6 Sup Temporal Ctx | 49.3 | 52.9 |
| Control 1 Temporal Ctx | 2.0 | 1.9 |
| Control 2 Temporal Ctx | 20.9 | 17.6 |
| Control 3 Temporal Ctx | 6.5 | 6.4 |
| Control 3 Temporal Ctx | 6.6 | 4.2 |
| Control (Path) 1 Temporal Ctx | 11.3 | 8.1 |
| Control (Path) 2 Temporal Ctx | 7.0 | 7.7 |
| Control (Path) 3 Temporal Ctx | 0.9 | 1.5 |
| Control (Path) 4 Temporal Ctx | 2.1 | 3.3 |
| AD 1 Occipital Ctx | 10.1 | 8.5 |
| AD 2 Occipital Ctx (Missing) | 0.0 | 0.0 |
| AD 3 Occipital Ctx | 0.3 | 3.2 |
| AD 4 Occipital Ctx | 14.1 | 11.8 |
| AD 5 Occipital Ctx | 20.2 | 20.7 |
| AD 6 Occipital Ctx | 20.2 | 18.4 |
| Control 1 Occipital Ctx | 1.2 | 1.3 |
| Control 2 Occipital Ctx | 27.9 | 31.6 |
| Control 3 Occipital Ctx | 5.6 | 4.3 |
| Control 4 Occipital Ctx | 9.3 | 6.3 |
| Control (Path) 1 Occipital Ctx | 23.5 | 20.3 |
| Control (Path) 2 Occipital Ctx | 2.1 | 3.1 |
| Control (Path) 3 Occipital Ctx | 1.9 | 2.5 |
| Control (Path) 4 Occipital Ctx | 3.1 | 3.2 |
| Control 1 Parietal Ctx | 2.0 | 2.5 |
| Control 2 Parietal Ctx | 33.9 | 32.3 |
| Control 3 Parietal Ctx | 4.6 | 6.4 |
| Control (Path) 1 Parietal Ctx | 12.4 | 11.7 |
| Control (Path) 2 Parietal Ctx | 6.7 | 7.5 |
| Control (Path) 3 Parietal Ctx | 1.4 | 1.2 |
| Control (Path) 4 Parietal Ctx | 6.9 | 5.2 |

Column A - Rel. Exp. (%) Ag6086, Run 248386491
Column B - Rel. Exp. (%) Ag6086, Run 275777034

Table AC. General_{—l screening panel v}1.5

TABLE AC
General_screening_panel_v1.5

| Tissue Name | A |
|---|---|
| Adipose | 29.9 |
| Melanoma* Hs688(A).T | 38.2 |
| Melanoma* Hs688(B).T | 17.6 |
| Melanoma* M14 | 55.5 |
| Melanoma* LOXIMVI | 9.1 |
| Melanoma* SK-MEL-5 | 62.4 |
| Squamous cell carcinoma SCC-4 | 1.9 |
| Testis Pool | 5.9 |
| Prostate ca.* (bone met) PC-3 | 0.9 |
| Prostate Pool | 1.0 |
| Placenta | 10.6 |
| Uterus Pool | 3.0 |
| Ovarian ca. OVCAR-3 | 1.4 |
| Ovarian ca. SK-OV-3 | 4.1 |
| Ovarian ca. OVCAR-4 | 1.6 |
| Ovarian ca. OVCAR-5 | 10.7 |
| Ovarian ca. IGROV-1 | 6.2 |
| Ovarian ca. OVCAR-8 | 0.5 |
| Ovary | 15.1 |
| Breast ca. MCF-7 | 0.9 |
| Breast ca. MDA-MB-231 | 4.8 |
| Breast ca. BT 549 | 24.5 |
| Breast ca. T47D | 0.0 |
| Breast ca. MDA-N | 11.7 |
| Breast Pool | 5.6 |
| Trachea | 8.7 |
| Lung | 7.4 |
| Fetal Lung | 15.8 |
| Lung ca. NCI-N417 | 0.4 |
| Lung ca. LX-1 | 1.8 |
| Lung ca. NCI-H146 | 2.7 |
| Lung ca. SHP-77 | 9.4 |
| Lung ca. A549 | 10.1 |
| Lung ca. NCI-H526 | 0.1 |
| Lung ca. NCI-H23 | 100.0 |
| Lung ca. NCI-H460 | 7.1 |
| Lung ca. HOP-62 | 1.5 |
| Lung ca. NCI-H522 | 0.1 |
| Liver | 1.0 |
| Fetal Liver | 4.8 |
| Liver ca. HepG2 | 1.4 |
| Kidney Pool | 4.5 |
| Fetal Kidney | 7.7 |
| Renal ca. 786-0 | 2.5 |
| Renal ca. A498 | 1.7 |
| Renal ca. ACHN | 6.6 |
| Renal ca. UO-31 | 2.8 |
| Renal ca. TK-10 | 1.4 |
| Bladder | 20.4 |
| Gastric ca. (liver met.) NCI-N87 | 2.4 |
| Gastric ca. KATO III | 3.7 |
| Colon ca. SW-948 | 0.3 |
| Colon ca. SW480 | 0.8 |
| Colon ca.* (SW480 met) SW620 | 0.4 |
| Colon ca. HT29 | 2.0 |
| Colon ca. HCT-116 | 0.6 |
| Colon ca. CaCo-2 | 9.8 |
| Colon cancer tissue | 15.9 |
| Colon ca. SW1116 | 0.5 |
| Colon ca. Colo-205 | 0.4 |
| Colon ca. SW-48 | 0.4 |
| Colon Pool | 4.2 |
| Small Intestine Pool | 1.2 |
| Stomach Pool | 3.7 |
| Bone Marrow Pool | 3.5 |
| Fetal Heart | 5.3 |
| Heart Pool | 1.9 |
| Lymph Node Pool | 4.0 |
| Fetal Skeletal Muscle | 3.4 |
| Skeletal Muscle Pool | 6.1 |
| Spleen Pool | 7.0 |
| Thymus Pool | 6.3 |
| CNS cancer (glio/astro) U87-MG | 6.2 |
| CNS cancer (glio/astro) U-118-MG | 10.2 |
| CNS cancer (neuro; met) SK-N-AS | 4.9 |
| CNS cancer (astro) SF-539 | 3.4 |
| CNS cancer (astro) SNB-75 | 13.9 |
| CNS cancer (glio) SNB-19 | 5.1 |
| CNS cancer (glio) SF-295 | 6.9 |
| Brain (Amygdala) Pool | 6.8 |
| Brain (cerebellum) | 8.0 |
| Brain (fetal) | 4.4 |
| Brain (Hippocampus) Pool | 8.4 |
| Cerebral Cortex Pool | 5.5 |
| Brain (Substantia nigra) Pool | 4.8 |
| Brain (Thalamus) Pool | 8.2 |
| Brain (whole) | 7.6 |
| Spinal Cord Pool | 7.6 |
| Adrenal Gland | 59.9 |
| Pituitary gland Pool | 0.5 |
| Salivary Gland | 2.6 |
| Thyroid (female) | 6.0 |
| Pancreatic ca. CAPAN2 | 3.1 |
| Pancreas Pool | 17.1 |

Column A - Rel. Exp. (%) Ag6086, Run 247775072

Table AD. Panel 5 Islet

TABLE AD
Panel 5 Islet

| Tissue Name | A |
|---|---|
| 97457_Patient-02go_adipose | 26.4 |
| 97476_Patient-07sk_skeletal muscle | 40.6 |
| 97477_Patient-07ut_uterus | 23.0 |
| 97478_Patient-07pl_placenta | 28.1 |
| 99167_Bayer Patient 1 | 59.5 |
| 97482_Patient-08ut_uterus | 24.3 |
| 97483_Patient-08pl_placenta | 3.9 |
| 97486_Patient-09sk_skeletal muscle | 5.3 |
| 97487_Patient-09ut_uterus | 21.0 |
| 97488_Patient-09pl_placenta | 16.4 |
| 97492_Patient-10ut_uterus | 29.5 |
| 97493_Patient-10pl_placenta | 40.9 |
| 97495_Patient-11go_adipose | 49.3 |
| 97496_Patient-11sk_skeletal muscle | 17.6 |
| 97497_Patient-11ut_uterus | 39.0 |
| 97498_Patient-11pl_placenta | 19.5 |
| 97500_Patient-12go_adipose | 84.7 |
| 97501_Patient-12sk_skeletal muscle | 67.8 |
| 97502_Patient-12ut_uterus | 25.7 |
| 97503_Patient-12pl_placenta | 37.4 |
| 94721_Donor 2 U - A_Mesenchymal Stem Cells | 26.2 |
| 94722_Donor 2 U - B_Mesenchymal Stem Cells | 19.9 |
| 94723_Donor 2 U - C Mesenchymal Stem Cells | 33.0 |
| 94709_Donor 2 AM - A_adipose | 4.7 |
| 94710_Donor 2 AM - B_adipose | 2.7 |
| 94711_Donor 2 AM - C_adipose | 3.4 |
| 94712_Donor 2 AD - A_adipose | 12.0 |
| 94713_Donor 2 AD - B_adipose | 12.6 |
| 94714_Donor 2 AD - C_adipose | 14.1 |
| 94742_Donor 3 U - A_Mesenchymal Stem Cells | 4.8 |
| 94743_Donor 3 U - B_Mesenchymal Stem Cells | 11.0 |
| 94730_Donor 3 AM - A_adipose | 5.6 |
| 94731_Donor 3 AM - B_adipose | 4.7 |
| 94732_Donor 3 AM - C_adipose | 4.0 |
| 94733_Donor 3 AD - A_adipose | 43.2 |
| 94734_Donor 3 AD - B_adipose | 15.5 |
| 94735_Donor 3 AD - C_adipose | 36.9 |
| 77138_Liver_HepG2untreated | 9.0 |
| 73556_Heart_Cardiac stromal cells (primary) | 20.0 |
| 81735_Small Intestine | 31.9 |
| 72409_Kidney_Proximal Convoluted Tubule | 38.7 |
| 82685_Small intestine_Duodenum | 4.6 |
| 90650_Adrenal_Adrenocortical adenoma | 68.3 |
| 72410_Kidney_HRCE | 79.0 |
| 72411_Kidney_HRE | 100.0 |
| 73139_Uterus_Uterine smooth muscle cells | 7.8 |

Column A - Rel. Exp. (%) Ag6086, Run 248045495

CNS_neurodegeneration_v1.0 Summary: Ag6086 Two experiments with same probe-primer sets are in excellent agreement. This panel confirms the expression of this gene at low levels in the brain in an independent group of individuals. This gene is found to be upregulated in the temporal cortex of Alzheimer's disease patients. This gene encodes serine/threonine-protein kinase Sgk protein. Blockade of SGK protein encoded by this gene may be of use in the treatment of this disease and decrease neuronal death.

General_screening_panel_v1.5 Summary: Ag6086 This gene shows ubiquitous expression with highest expression in a lung cancer NCI-H23 cell line (CT=25.5). Moderate to high levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate to high levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Panel 5 Islet Summary: Ag6086 This gene shows wide spread expression in this panel with highest expression in kidney (CT=27.8). The moderate expression of SGK1 in adipose, kidney and skeletal muscle is consistent with literature and the expression pattern seen in panel 1.5. Notably, SGK1 is also expressed in pancreatic islets. SGK is the serine/threonine kinase implicated in development of several diabetic complications, specifically hypertension and diabetic nephropathy (Lang, F.; et al., 2000Proc. Nat. Acad. Sci. 97: 8157-8162. (PMID: 10884438); Lang F, et al. 2001, Sci STKE 108:RE17 (PMID: 11707620). It has been shown that SGK phosphorylates the cytoplasmic domains of several ion channels/transporters modulating their activity, and/or endocytosis rate. SGK gene has been mapped to the region 6q23. Interestingly, duplication of this chromosomal region causes transient neonatal diabetes mellitus characterized by hyperglycemia and predisposition toward type 2 diabetes (Temple, I. K.; et al. 1996, Hum. Molec. Genet. 5: 1117-1124. (PMID: 8842729). Together with the localization of SGK gene in loci for neonatal diabetes, the data suggest that SGK1 might modulate insulin secretion.

B. CG50183-01: Chemokine Receptor

Expression of gene CG50183-01 was assessed using the primer-probe set Gpcr09, described in Table BA. Results of the RTQ-PCR runs are shown in Table BB.

TABLE BA

Probe Name Gpcr09

| | | | | |
|---|---|---|---|---|
| | | | Start | SEQ ID |
| Primers | Sequences | Length | Position | No |

| | | | | |
|---|---|---|---|---|
| Forward | 5′-tgagcaaacgcatggacatc-3′ | 20 | 826 | 101 |
| Probe | TET-5′-ccatccaagtcacagaaagcatcgcact-3′-TAMRA | 28 | 847 | 102 |
| Reverse | 5′-tgggttgaggcagctgtga-3′ | 19 | 878 | 103 |

TABLE BB
Panel 1

| Tissue Name | A |
|---|---|
| Endothelial cells | 0.8 |
| Endothelial cells (treated) | 0.8 |
| Pancreas | 17.4 |
| Pancreatic ca. CAPAN 2 | 2.5 |
| Adrenal gland | 17.2 |
| Thyroid | 8.5 |
| Salivary gland | 39.2 |
| Pituitary gland | 25.3 |
| Brain (fetal) | 2.0 |
| Brain (whole) | 3.3 |
| Brain (amygdala) | 8.2 |
| Brain (cerebellum) | 10.8 |
| Brain (hippocampus) | 7.3 |
| Brain (substantia nigra) | 27.2 |
| Brain (thalamus) | 4.0 |
| Brain (hypothalamus) | 100.0 |
| Spinal cord | 7.8 |
| glio/astro U87-MG | 9.9 |
| glio/astro U-118-MG | 6.7 |
| astrocytoma SW1783 | 4.4 |
| neuro*; met SK-N-AS | 5.5 |
| astrocytoma SF-539 | 9.0 |
| astrocytoma SNB-75 | 4.6 |
| glioma SNB-19 | 14.9 |
| glioma U251 | 11.7 |
| glioma SF-295 | 13.6 |
| Heart | 56.3 |
| Skeletal muscle | 6.2 |
| Bone marrow | 4.8 |
| Thymus | 22.1 |
| Spleen | 12.6 |
| Lymph node | 19.2 |
| Colon (ascending) | 37.1 |
| Stomach | 24.5 |
| Small intestine | 55.1 |
| Colon ca. SW480 | 3.8 |
| Colon ca.* SW620 (SW480 met) | 5.1 |
| Colon ca. HT29 | 8.4 |
| Colon ca. HCT-116 | 8.1 |
| Colon ca. CaCo-2 | 11.2 |
| Colon ca. HCT-15 | 8.3 |
| Colon ca. HCC-2998 | 13.5 |
| Gastric ca. * (liver met) NCI-N87 | 36.3 |
| Bladder | 30.6 |
| Trachea | 19.6 |
| Kidney | 24.1 |
| Kidney (fetal) | 14.4 |
| Renal ca. 786-0 | 5.8 |
| Renal ca. A498 | 19.2 |
| Renal ca. RXF 393 | 5.0 |
| Renal ca. ACHN | 14.8 |
| Renal ca. UO-31 | 11.1 |
| Renal ca. TK-10 | 13.4 |
| Liver | 18.4 |
| Liver (fetal) | 7.4 |
| Liver ca. (hepatoblast) HepG2 | 28.3 |
| Lung | 0.0 |
| Lung (fetal) | 9.1 |
| Lung ca. (small cell) LX-1 | 7.9 |
| Lung ca. (small cell) NCI-H69 | 13.2 |
| Lung ca. (s. cell var.) SHP-77 | 2.2 |
| Lung ca. (large cell) NCI-H460 | 25.5 |
| Lung ca. (non-sm. cell) A549 | 20.9 |
| Lung ca. (non-s. cell) NCI-H23 | 3.6 |
| Lung ca. (non-s. cell) HOP-62 | 12.5 |
| Lung ca. (non-s. cl) NCI-H522 | 19.9 |
| Lung ca. (squam.) SW 900 | 9.5 |
| Lung ca. (squam.) NCI-H596 | 20.2 |
| Mammary gland | 57.8 |
| Breast ca.* (pl. ef) MCF-7 | 7.6 |
| Breast ca.* (pl. ef) MDA-MB-231 | 1.2 |
| Breast ca.* (pl. ef) T47D | 34.4 |
| Breast ca. BT-549 | 4.1 |
| Breast ca. MDA-N | 51.8 |
| Ovary | 6.9 |
| Ovarian ca. OVCAR-3 | 15.2 |
| Ovarian ca. OVCAR-4 | 2.6 |
| Ovarian ca. OVCAR-5 | 22.2 |
| Ovarian ca. OVCAR-8 | 36.1 |
| Ovarian ca. IGROV-1 | 12.6 |
| Ovarian ca. (ascites) SK-OV-3 | 7.5 |
| Uterus | 21.8 |
| Placenta | 7.5 |
| Prostate | 19.5 |
| Prostate ca.* (bone met) PC-3 | 12.8 |
| Testis | 14.8 |
| Melanoma Hs688(A).T | 7.5 |
| Melanoma* (met) Hs688(B).T | 11.3 |
| Melanoma UACC-62 | 5.0 |
| Melanoma M14 | 10.1 |
| Melanoma LOX IMVI | 1.6 |
| Melanoma* (met) SK-MEL-5 | 15.7 |
| Melanoma SK-MEL-28 | 25.5 |

Column A - Rel. Exp. (%) Gpcr09, Run 109665126

Panel 1 Summary: Gpcr09 Highest expression is seen in hypothalamus (CT=25). Prominent levels of expression are seen primarily in normal tissues, including mammary gland, heart, colon, stomach, and substantia nigra.

C. CG50249-01: Voltage-Gated Potassium Channel Protein KV3.2 Like

Expression of gene CG50249-01 was assessed using the primer-probe set Ag2503, described in Table CA. Results of the RTQ-PCR runs are shown in Tables CB, CC, CD and CE.

Table CA. Probe Name Ag 2503

TABLE CA

Probe Name Ag2503

| | | | | |
|---|---|---|---|---|
| | | | Start | SEQ ID |
| Primers | Sequences | Length | Position | No |

| | | | | |
|---|---|---|---|---|
| Forward | 5′-gaggctctctccagtaacatca-3′ | 22 | 1851 | 104 |
| Probe | TET-5′-actctccttgtcctctgaggcgctct-3′-TAMRA | 26 | 1880 | 105 |
| Reverse | 5′-gcagtttggttgtttggtttac-3′ | 22 | 1929 | 106 |

Table CB. CNS_neurodegeneration_v1.0

TABLE CB
CNS_neurodegeneration_v1.0

| Tissue Name | A | B |
|---|---|---|
| AD 1 Hippo | 4.1 | 4.4 |
| AD 2 Hippo | 10.5 | 9.3 |
| AD 3 Hippo | 1.3 | 1.2 |
| AD 4 Hippo | 1.6 | 1.8 |
| AD 5 Hippo | 100.0 | 100.0 |
| AD 6 Hippo | 16.8 | 16.5 |
| Control 2 Hippo | 13.4 | 14.5 |
| Control 4 Hippo | 1.1 | 0.8 |
| Control (Path) 3 Hippo | 0.5 | 0.6 |
| AD 1 Temporal Ctx | 3.9 | 4.1 |
| AD 2 Temporal Ctx | 19.3 | 18.9 |
| AD 3 Temporal Ctx | 1.4 | 1.1 |
| AD 4 Temporal Ctx | 9.0 | 9.4 |
| AD 5 Inf Temporal Ctx | 84.1 | 83.5 |
| AD 5 Sup Temporal Ctx | 19.5 | 17.0 |
| AD 6 Inf Temporal Ctx | 18.0 | 20.2 |
| AD 6 Sup Temporal Ctx | 28.7 | 26.4 |
| Control 1 Temporal Ctx | 1.0 | 0.7 |
| Control 2 Temporal Ctx | 31.6 | 42.6 |
| Control 3 Temporal Ctx | 9.0 | 11.4 |
| Control 3 Temporal Ctx | 2.2 | 1.9 |
| Control (Path) 1 Temporal Ctx | 52.5 | 54.7 |
| Control (Path) 2 Temporal Ctx | 32.1 | 37.9 |
| Control (Path) 3 Temporal Ctx | 0.6 | 1.0 |
| Control (Path) 4 Temporal Ctx | 26.8 | 28.1 |
| AD 1 Occipital Ctx | 12.1 | 12.5 |
| AD 2 Occipital Ctx (Missing) | 0.0 | 0.0 |
| AD 3 Occipital Ctx | 1.8 | 2.1 |
| AD 4 Occipital Ctx | 13.8 | 10.4 |
| AD 5 Occipital Ctx | 45.7 | 48.3 |
| AD 6 Occipital Ctx | 15.5 | 12.6 |
| Control 1 Occipital Ctx | 0.2 | 0.0 |
| Control 2 Occipital Ctx | 54.0 | 56.3 |
| Control 3 Occipital Ctx | 11.2 | 10.7 |
| Control 4 Occipital Ctx | 0.5 | 0.5 |
| Control (Path) 1 Occipital Ctx | 76.8 | 78.5 |
| Control (Path) 2 Occipital Ctx | 9.5 | 8.4 |
| Control (Path) 3 Occipital Ctx | 0.2 | 0.3 |
| Control (Path) 4 Occipital Ctx | 13.0 | 14.1 |
| Control 1 Parietal Ctx | 1.0 | 1.1 |
| Control 2 Parietal Ctx | 26.6 | 26.4 |
| Control 3 Parietal Ctx | 18.2 | 15.4 |
| Control (Path) 1 Parietal Ctx | 71.2 | 73.7 |
| Control (Path) 2 Parietal Ctx | 17.7 | 16.6 |
| Control (Path) 3 Parietal Ctx | 0.6 | 0.5 |
| Control (Path) 4 Parietal Ctx | 44.1 | 43.2 |

Column A - Rel. Exp. (%) Ag2503, Run 208779478
Column B - Rel. Exp. (%) Ag2503, Run 253338938

Table CC. General screening_panel_v1.4

TABLE CC
General_screening_panel_v1.4

| Tissue Name | A | B |
|---|---|---|
| Adipose | 0.0 | 0.0 |
| Melanoma* Hs688(A).T | 0.0 | 0.0 |
| Melanoma* Hs688(B).T | 0.0 | 0.0 |
| Melanoma* M14 | 0.0 | 0.0 |
| Melanoma* LOXIMVI | 0.0 | 0.1 |
| Melanoma* SK-MEL-5 | 0.0 | 0.1 |
| Squamous cell carcinoma SCC-4 | 0.0 | 0.0 |
| Testis Pool | 0.2 | 0.3 |
| Prostate ca.* (bone met) PC-3 | 0.0 | 0.0 |
| Prostate Pool | 6.4 | 7.8 |
| Placenta | 0.0 | 0.0 |
| Uterus Pool | 0.0 | 0.0 |
| Ovarian ca. OVCAR-3 | 0.0 | 0.0 |
| Ovarian ca. SK-OV-3 | 0.0 | 0.1 |
| Ovarian ca. OVCAR-4 | 0.0 | 0.0 |
| Ovarian ca. OVCAR-5 | 8.4 | 7.2 |
| Ovarian ca. IGROV-1 | 0.0 | 0.0 |
| Ovarian ca. OVCAR-8 | 0.0 | 0.0 |
| Ovary | 0.0 | 0.1 |
| Breast ca. MCF-7 | 0.0 | 0.2 |
| Breast ca. MDA-MB-231 | 0.0 | 0.0 |
| Breast ca. BT 549 | 0.0 | 0.0 |
| Breast ca. T47D | 8.1 | 15.4 |
| Breast ca. MDA-N | 0.0 | 0.0 |
| Breast Pool | 0.9 | 0.5 |
| Trachea | 0.2 | 0.4 |
| Lung | 0.0 | 0.0 |
| Fetal Lung | 0.0 | 0.1 |
| Lung ca. NCI-N417 | 0.0 | 0.0 |
| Lung ca. LX-1 | 0.0 | 0.0 |
| Lung ca. NCI-H146 | 1.8 | 1.8 |
| Lung ca. SHP-77 | 0.5 | 0.5 |
| Lung ca. A549 | 0.0 | 0.0 |
| Lung ca. NCI-H526 | 0.0 | 0.0 |
| Lung ca. NCI-H23 | 0.0 | 0.9 |
| Lung ca. NCI-H460 | 2.0 | 0.1 |
| Lung ca. HOP-62 | 0.1 | 0.0 |
| Lung ca. NCI-H522 | 0.0 | 0.0 |
| Liver | 0.1 | 0.0 |
| Fetal Liver | 0.0 | 0.3 |
| Liver ca. HepG2 | 0.0 | 0.0 |
| Kidney Pool | 0.0 | 0.1 |
| Fetal Kidney | 1.1 | 2.2 |
| Renal ca. 786-0 | 0.0 | 0.0 |
| Renal ca. A498 | 0.0 | 0.0 |
| Renal ca. ACHN | 0.4 | 0.0 |
| Renal ca. UO-31 | 0.0 | 0.0 |
| Renal ca. TK-10 | 0.0 | 0.0 |
| Bladder | 0.1 | 0.1 |
| Gastric ca. (liver met.) NCI-N87 | 0.1 | 0.1 |
| Gastric ca. KATO III | 0.0 | 0.0 |
| Colon ca. SW-948 | 0.0 | 0.0 |
| Colon ca. SW480 | 0.0 | 0.1 |
| Colon ca.* (SW480 met) SW620 | 0.0 | 0.0 |
| Colon ca. HT29 | 0.1 | 0.1 |
| Colon ca. HCT-116 | 0.0 | 0.0 |
| Colon ca. CaCo-2 | 0.0 | 0.0 |
| Colon cancer tissue | 0.1 | 0.2 |
| Colon ca. SW1116 | 0.0 | 0.0 |
| Colon ca. Colo-205 | 0.0 | 0.0 |
| Colon ca. SW-48 | 0.0 | 0.0 |
| Colon Pool | 0.2 | 0.1 |
| Small Intestine Pool | 0.2 | 0.4 |
| Stomach Pool | 0.2 | 0.0 |
| Bone Marrow Pool | 0.0 | 0.0 |
| Fetal Heart | 0.0 | 0.0 |
| Heart Pool | 0.0 | 0.1 |
| Lymph Node Pool | 0.1 | 0.1 |
| Fetal Skeletal Muscle | 0.1 | 0.0 |
| Skeletal Muscle Pool | 0.0 | 0.1 |
| Spleen Pool | 0.0 | 0.0 |
| Thymus Pool | 0.4 | 0.7 |
| CNS cancer (glio/astro) U87-MG | 0.0 | 0.0 |
| CNS cancer (glio/astro) U-118-MG | 0.1 | 0.1 |
| CNS cancer (neuro; met) SK-N-AS | 0.0 | 0.0 |
| CNS cancer (astro) SF-539 | 0.0 | 0.0 |
| CNS cancer (astro) SNB-75 | 0.0 | 0.0 |
| CNS cancer (glio) SNB-19 | 0.0 | 0.0 |
| CNS cancer (glio) SF-295 | 0.0 | 0.0 |
| Brain (Amygdala) Pool | 55.9 | 49.7 |
| Brain (cerebellum) | 1.1 | 1.1 |
| Brain (fetal) | 25.9 | 38.4 |
| Brain (Hippocampus) Pool | 31.0 | 35.8 |
| Cerebral Cortex Pool | 100.0 | 80.7 |
| Brain (Substantia nigra) Pool | 64.2 | 64.6 |
| Brain (Thalamus) Pool | 97.3 | 100.0 |
| Brain (whole) | 66.9 | 65.5 |
| Spinal Cord Pool | 6.4 | 5.3 |
| Adrenal Gland | 0.0 | 0.0 |
| Pituitary gland Pool | 6.6 | 5.5 |
| Salivary Gland | 0.2 | 0.1 |
| Thyroid (female) | 0.0 | 0.0 |
| Pancreatic ca. CAPAN2 | 0.0 | 0.1 |
| Pancreas Pool | 0.2 | 0.7 |

Column A - Rel. Exp. (%) Ag2503, Run 208015585
Column B - Rel. Exp. (%) Ag2503, Run 212142287

Table CD. Panel 1.3D

TABLE CD
Panel 1.3D

| Tissue Name | A | B |
|---|---|---|
| Liver adenocarcinoma | 0.0 | 0.0 |
| Pancreas | 0.0 | 0.0 |
| Pancreatic ca. CAPAN 2 | 0.0 | 0.0 |
| Adrenal gland | 0.4 | 0.3 |
| Thyroid | 0.0 | 0.0 |
| Salivary gland | 0.1 | 0.1 |
| Pituitary gland | 6.0 | 3.0 |
| Brain (fetal) | 9.6 | 12.1 |
| Brain (whole) | 66.9 | 80.1 |
| Brain (amygdala) | 27.0 | 21.2 |
| Brain (cerebellum) | 0.8 | 2.0 |
| Brain (hippocampus) | 100.0 | 33.2 |
| Brain (substantia nigra) | 5.5 | 5.9 |
| Brain (thalamus) | 93.3 | 100.0 |
| Cerebral Cortex | 84.7 | 23.7 |
| Spinal cord | 0.8 | 0.9 |
| glio/astro U87-MG | 0.0 | 0.0 |
| glio/astro U-118-MG | 0.1 | 0.0 |
| astrocytoma SW1783 | 0.0 | 0.0 |
| neuro*; met SK-N-AS | 0.0 | 0.0 |
| astrocytoma SF-539 | 0.0 | 0.0 |
| astrocytoma SNB-75 | 0.0 | 0.0 |
| glioma SNB-19 | 0.1 | 0.0 |
| glioma U251 | 0.0 | 0.0 |
| glioma SF-295 | 0.1 | 0.0 |
| Heart (fetal) | 0.0 | 0.0 |
| Heart | 0.0 | 0.0 |
| Skeletal muscle (fetal) | 0.3 | 1.3 |
| Skeletal muscle | 0.0 | 0.0 |
| Bone marrow | 0.0 | 0.0 |
| Thymus | 0.0 | 0.0 |
| Spleen | 0.0 | 0.5 |
| Lymph node | 0.0 | 0.0 |
| Colorectal | 0.1 | 0.0 |
| Stomach | 0.0 | 0.0 |
| Small intestine | 0.1 | 0.2 |
| Colon ca. SW480 | 0.0 | 0.0 |
| Colon ca.* SW620 (SW480 met) | 0.0 | 0.0 |
| Colon ca. HT29 | 0.1 | 0.0 |
| Colon ca. HCT-116 | 0.0 | 0.0 |
| Colon ca. CaCo-2 | 0.0 | 0.0 |
| Colon ca. tissue (ODO3866) | 0.1 | 0.2 |
| Colon ca. HCC-2998 | 0.0 | 0.1 |
| Gastric ca.* (liver met) NCI-N87 | 0.0 | 0.1 |
| Bladder | 0.0 | 0.0 |
| Trachea | 0.1 | 0.0 |
| Kidney | 0.0 | 0.0 |
| Kidney (fetal) | 0.0 | 0.2 |
| Renal ca. 786-0 | 0.0 | 0.0 |
| Renal ca. A498 | 0.0 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.0 | 0.0 |
| Liver (fetal) | 0.0 | 0.0 |
| Liver ca. (hepatoblast) HepG2 | 0.0 | 0.0 |
| Lung | 0.0 | 0.0 |
| Lung (fetal) | 0.0 | 0.0 |
| Lung ca. (small cell) LX-1 | 0.0 | 0.1 |
| Lung ca. (small cell) NCI-H69 | 0.4 | 0.0 |
| Lung ca. (s. cell var.) SHP-77 | 0.2 | 0.1 |
| Lung ca. (large cell) NCI-H460 | 0.0 | 0.2 |
| Lung ca. (non-sm. cell) A549 | 0.0 | 0.0 |
| Lung ca. (non-s. cell) NCI-H23 | 0.0 | 0.1 |
| Lung ca. (non-s. cell) HOP-62 | 0.1 | 0.0 |
| Lung ca. (non-s. cl) NCI-H522 | 0.0 | 0.0 |
| Lung ca. (squam.) SW 900 | 0.0 | 0.0 |
| Lung ca. (squam.) NCI-H596 | 0.2 | 0.7 |
| Mammary gland | 5.1 | 1.7 |
| Breast ca.* (pl. ef) MCF-7 | 0.1 | 0.0 |
| Breast ca.* (pl. ef) MDA-MB-231 | 0.0 | 0.0 |
| Breast ca.* (pl. ef) T47D | 0.0 | 0.0 |
| Breast ca. BT-549 | 0.0 | 0.0 |
| Breast ca. MDA-N | 0.0 | 0.0 |
| Ovary | 0.0 | 0.0 |
| Ovarian ca. OVCAR-3 | 0.0 | 0.0 |
| Ovarian ca. OVCAR-4 | 0.0 | 0.0 |
| Ovarian ca. OVCAR-5 | 1.5 | 1.0 |
| Ovarian ca. OVCAR-8 | 0.0 | 0.0 |
| Ovarian ca. IGROV-1 | 0.0 | 0.0 |
| Ovarian ca.* (ascites) SK-OV-3 | 0.0 | 0.3 |
| Uterus | 0.2 | 0.0 |
| Placenta | 0.0 | 0.0 |
| Prostate | 3.2 | 1.7 |
| Prostate ca.* (bone met) PC-3 | 0.0 | 0.0 |
| Testis | 0.0 | 0.0 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |
| Adipose | 0.0 | 0.0 |

Column A - Rel. Exp. (%) Ag2503, Run 160838046
Column B - Rel. Exp. (%) Ag2503, Run 165519979

TABLE CE
Panel CNS_1

| Tissue Name | A |
|---|---|
| BA4 Control | 31.9 |
| BA4 Control2 | 65.1 |
| BA4 Alzheimer's2 | 4.0 |
| BA4 Parkinson's | 70.2 |
| BA4 Parkinson's2 | 100.0 |
| BA4 Huntington's | 41.2 |
| BA4 Huntington's2 | 2.1 |
| BA4 PSP | 5.3 |
| BA4 PSP2 | 24.7 |
| BA4 Depression | 10.8 |
| BA4 Depression2 | 9.0 |
| BA7 Control | 42.3 |
| BA7 Control2 | 40.9 |
| BA7 Alzheimer's2 | 6.4 |
| BA7 Parkinson's | 18.8 |
| BA7 Parkinson's2 | 46.3 |
| BA7 Huntington's | 57.8 |
| BA7 Huntington's2 | 52.9 |
| BA7 PSP | 35.4 |
| BA7 PSP2 | 25.9 |
| BA7 Depression | 5.2 |
| BA9 Control | 23.2 |
| BA9 Control2 | 88.3 |
| BA9 Alzheimer's | 4.9 |
| BA9 Alzheimer's2 | 12.8 |
| BA9 Parkinson's | 25.5 |
| BA9 Parkinson's2 | 61.6 |
| BA9 Huntington's | 42.3 |
| BA9 Huntington's2 | 12.4 |
| BA9 PSP | 9.2 |
| BA9 PSP2 | 4.0 |
| BA9 Depression | 3.5 |
| BA9 Depression2 | 8.4 |
| BA17 Control | 58.2 |
| BA17 Control2 | 62.4 |
| BA17 Alzheimer's2 | 7.3 |
| BA17 Parkinson's | 31.9 |
| BA17 Parkinson's2 | 57.8 |
| BA17 Huntington's | 32.8 |
| BA17 Huntington's2 | 12.8 |
| BA17 Depression | 2.6 |
| BA17 Depression2 | 23.3 |
| BA17 PSP | 22.2 |
| BA17 PSP2 | 10.1 |
| Sub Nigra Control | 14.8 |
| Sub Nigra Control2 | 9.9 |
| Sub Nigra Alzheimer's2 | 2.9 |
| Sub Nigra Parkinson's2 | 18.9 |
| Sub Nigra Huntington's | 10.5 |
| Sub Nigra Huntington's2 | 10.7 |
| Sub Nigra PSP2 | 0.9 |
| Sub Nigra Depression | 0.5 |
| Sub Nigra Depression2 | 3.1 |
| Glob Palladus Control | 0.5 |
| Glob Palladus Control2 | 1.4 |
| Glob Palladus Alzheimer's | 3.1 |
| Glob Palladus Alzheimer's2 | 1.0 |
| Glob Palladus Parkinson's | 39.8 |
| Glob Palladus Parkinson's2 | 0.8 |
| Glob Palladus PSP | 0.0 |
| Glob Palladus PSP2 | 0.8 |
| Glob Palladus Depression | 0.2 |
| Temp Pole Control | 17.3 |
| Temp Pole Control2 | 50.0 |
| Temp Pole Alzheimer's | 1.7 |
| Temp Pole Alzheimer's2 | 3.0 |
| Temp Pole Parkinson's | 19.1 |
| Temp Pole Parkinson's2 | 18.4 |
| Temp Pole Huntington's | 34.2 |
| Temp Pole PSP | 4.1 |
| Temp Pole PSP2 | 2.8 |
| Temp Pole Depression2 | 3.8 |
| Cing Gyr Control | 73.2 |
| Cing Gyr Control2 | 23.8 |
| Cing Gyr Alzheimer's | 19.6 |
| Cing Gyr Alzheimer's2 | 3.7 |
| Cing Gyr Parkinson's | 21.0 |
| Cing Gyr Parkinson's2 | 26.1 |
| Cing Gyr Huntington's | 49.7 |
| Cing Gyr Huntington's2 | 11.3 |
| Cing Gyr PSP | 5.6 |
| Cing Gyr PSP2 | 3.1 |
| Cing Gyr Depression | 2.5 |
| Cing Gyr Depression2 | 7.3 |

Column A - Rel. Exp. (%) Ag2503, Run 171656392

CNS_neurodegeneration_v1.0 Summary: Data obtained from RTQPCR assays performed for this panel was analyized using multivariate analysis (ANOVA). The multivariate results obtained using Ag2503 exhibits this gene has high brain—preferential expression in the hippocampus, cortex, amygdala, substantia nigra and thalamus. These regions are susceptible to the neurodegeneration associated with Alzheimer's Disease, Parkinson's disease, Huntington's disease and other pathological neurodegenerative conditions. This gene encodes a protein that is homologous to a potassium channel. Potassium channels have been implicated in neurodegenerative diseases, including Alzheimer's Disease. It has been suggested that modulating these channels to reduce outward K+ current may provide an approach to reducing neuronal degeneration in patients with Alzheimer's disease. Therefore, agents that modulate the function of this gene product could potentially reduce neuronal degeneration in patients with Alzheimer's Disease and other neurodegenerative diseases.

In addition, defective potassium channels are known to cause several CNS disorders, including epilepsy and episodic ataxia with myokymia. Therefore, modulation of the expression or function of this gene product may potentially be useful as a treatment for the symptoms produced by ataxia and epilepsy.

References:

Jhamandas J H, et al. J Neurophysiol September 2001;86(3):1312-20

Chi X, et al. Neurosci Lett Aug. 18, 2000 ;290(1):9-12

Piccini A, et al. Neuroreport May 15, 2000;11(7):1375-9

Yu S P, et al. Neurobiol Dis August 1998;5(2):81-8

Colom L V, et al. J Neurochem May 1998;70(5):1925-34

General_screening_panel_v1.4 Summary: Ag2503 Two experiments with the same probe and primer set produce reults that are in excellent agreement, with highest expression in the brain. Please see CNS_neurodegeneration_v1.0 for discussion of potential role in the central nervous system.

There is also moderate to low expression in normal prostate and in cell lines derived from breast, lung, and ovarian cancer. Thus, this expression could be used as a diagnostic marker for the presence of cancers in any of those tissues. Furthermore,inhibition of the activity of the gene product by antibodies or small molecule inhibitors could potentially be used as a treatment of these cancers.

In both experiments, there is also significantly higher levels of expression in the fetal kidney (CTs=30-31) when compared to the adult kidney (CTs=35-36). Thus, expression of this gene could be used to differentiate between adult and fetal sources of this tissue. Furthermore, the higher levels of expression in the fetal kidney suggest that this gene product may be involved in the development of this organ. Thus, therapeutic modulation of the expression or function of the protein encoded by this gene may be useful in the treatment of diseases of the kidney.

Among tissues with metabolic function, the expression of this potassium channel homolog is highest in the pituitary gland and shows very good concordance between the two independent runs. Potassium channels are involved in regulation of secretion in pituitary cells and their modulation by therapeutics such as small molecule inhibitors or antibodies could be used to modulate specific secretory activities in the pituitary.

Panel 1.3D Summary: Ag2503 Two experiments with the same probe and primer set produce results that are in very good agreement, with highest expression in both experiments seen in the brain. Please see CNS_neurodegeneration_v1.0.

Moderate to low expression is also observed in some cancer cell lines (lung and ovary) as well as normal prostate and breast. Thus, this expression could be used as a diagnostic marker for lung and ovarian cancers. Furthermore, inhibition of the activity of this gene product through the application of antibodies or small molecule inhibitors could effective in the treatment of lung or ovarian cancers.

As in panel 1.4, expression of this gene among metabolic tissues is highest in the pituitary. Significantly lower levels of expression are seen in the adrenal gland and in fetal skeletal muscle. Potassium channels are involved in regulation of secretion in pituitary cells and their modulation by therapeutics such as small molecule inhibitors or antibodies could be used to modulate specific secretory activities in the pituitary, as well as in other tissues.

In both experiments, there is also significantly higher levels of expression in the fetal skeletal muscle(CTs=33) when compared to expression in adult skeletal muscle(CTs=40). Thus, expression of this gene could be used to differentiate between adult and fetal sources of this tissue. Furthermore, the higher levels of expression in fetal skeletal muscle suggest that this gene product may be involved in the development of the skeletal muscle in the fetus. Thus, therapeutic modulation of the expression or function of the protein encoded by this gene may be useful in the adult to restore mass or function to weak or dystrophic muscle.

Panel CNS_{—}1 Summary: Ag2503 Ubiquitous expression in this panel confirms the presence in the brain of this protein product. See NS_neurodegeneration_v1.0 for discussion of potential role in the central nervous system.

D. CG54236-02: Cysteinyl Leukotriene CYSLT2 Receptor

Expression of gene CG54236-02 was assessed using the primer-probe set Ag2695, described in Table DA. Results of the RTQ-PCR runs are shown in Tables DB, DC, DD, DE and DF.

TABLE DA

Probe Name Ag2695

| | | | | |
|---|---|---|---|---|
| | | | Start | SEQ ID |
| Primers | Sequences | Length | Position | No |

| | | | | |
|---|---|---|---|---|
| Forward | 5′-gggaaatgggttgtccatatat-3′ | 22 | 266 | 107 |
| Probe | TET-5′-tcctgcagccttataagaagtccaca-3′-TAMRA | 26 | 292 | 108 |
| Reverse | 5′-atctgaaatggccagatttagc-3′ | 22 | 335 | 109 |

TABLE DB
AI_comprehensive_panel_v1.0

| Tissue Name | A | B |
|---|---|---|
| 110967 COPD-F | 7.1 | 2.5 |
| 110980 COPD-F | 6.2 | 0.0 |
| 110968 COPD-M | 6.9 | 7.9 |
| 110977 COPD-M | 12.3 | 15.2 |
| 110989 Emphysema-F | 45.1 | 28.5 |
| 110992 Emphysema-F | 15.4 | 7.6 |
| 110993 Emphysema-F | 11.9 | 4.8 |
| 110994 Emphysema-F | 2.5 | 0.0 |
| 110995 Emphysema-F | 12.9 | 38.4 |
| 110996 Emphysema-F | 12.2 | 0.0 |
| 110997 Asthma-M | 4.2 | 3.2 |
| 111001 Asthma-F | 46.3 | 25.2 |
| 111002 Asthma-F | 52.5 | 27.5 |
| 111003 Atopic Asthma-F | 58.2 | 46.0 |
| 111004 Atopic Asthma-F | 42.3 | 53.2 |
| 111005 Atopic Asthma-F | 26.4 | 26.8 |
| 111006 Atopic Asthma-F | 7.1 | 13.7 |
| 111417 Allergy-M | 20.0 | 18.8 |
| 112347 Allergy-M | 0.0 | 0.0 |
| 112349 Normal Lung-F | 0.0 | 0.0 |
| 112357 Normal Lung-F | 34.4 | 28.9 |
| 112354 Normal Lung-M | 98.6 | 55.5 |
| 112374 Crohns-F | 5.6 | 7.2 |
| 112389 Match Control Crohns-F | 11.0 | 5.2 |
| 112375 Crohns-F | 7.8 | 2.5 |
| 112732 Match Control Crohns-F | 43.5 | 18.6 |
| 112725 Crohns-M | 3.2 | 0.0 |
| 112387 Match Control Crohns-M | 2.1 | 8.0 |
| 112378 Crohns-M | 0.0 | 0.0 |
| 112390 Match Control Crohns-M | 28.1 | 32.5 |
| 112726 Crohns-M | 23.2 | 25.3 |
| 112731 Match Control Crohns-M | 20.0 | 15.3 |
| 112380 Ulcer Col-F | 11.3 | 20.7 |
| 112734 Match Control Ulcer Col-F | 82.9 | 43.2 |
| 112384 Ulcer Col-F | 24.7 | 25.9 |
| 112737 Match Control Ulcer Col-F | 8.3 | 3.7 |
| 112386 Ulcer Col-F | 2.2 | 3.6 |
| 112738 Match Control Ulcer Col-F | 10.5 | 4.1 |
| 112381 Ulcer Col-M | 1.5 | 0.0 |
| 112735 Match Control Ulcer Col-M | 11.7 | 4.5 |
| 112382 Ulcer Col-M | 25.5 | 16.6 |
| 112394 Match Control Ulcer Col-M | 1.3 | 2.9 |
| 112383 Ulcer Col-M | 12.2 | 11.7 |
| 112736 Match Control Ulcer Col-M | 15.7 | 5.6 |
| 112423 Psoriasis-F | 21.2 | 18.2 |
| 112427 Match Control Psoriasis-F | 54.0 | 53.2 |
| 112418 Psoriasis-M | 0.0 | 6.4 |
| 112723 Match Control Psoriasis-M | 0.0 | 0.0 |
| 112419 Psoriasis-M | 12.5 | 12.5 |
| 112424 Match Control Psoriasis-M | 19.5 | 11.0 |
| 112420 Psoriasis-M | 85.9 | 75.3 |
| 112425 Match Control Psoriasis-M | 48.0 | 58.2 |
| 104689 (MF) OA Bone-Backus | 39.0 | 59.9 |
| 104690 (MF) Adj “Normal” Bone-Backus | 21.8 | 27.7 |
| 104691 (MF) OA Synovium-Backus | 77.4 | 68.3 |
| 104692 (BA) OA Cartilage-Backus | 0.0 | 0.0 |
| 104694 (BA) OA Bone-Backus | 48.3 | 47.0 |
| 104695 (BA) Adj “Normal” Bone-Backus | 24.8 | 21.5 |
| 104696 (BA) OA Synovium-Backus | 81.8 | 49.7 |
| 104700 (SS) OA Bone-Backus | 26.8 | 15.2 |
| 104701 (SS) Adj “Normal” Bone-Backus | 13.5 | 11.4 |
| 104702 (SS) OA Synovium-Backus | 62.0 | 49.0 |
| 117093 OA Cartilage Rep7 | 89.5 | 59.0 |
| 112672 OA Bone5 | 16.7 | 38.2 |
| 112673 OA Synovium5 | 15.7 | 11.3 |
| 112674 OA Synovial Fluid cells5 | 0.0 | 12.9 |
| 117100 OA Cartilage Rep14 | 7.6 | 6.1 |
| 112756 OA Bone9 | 22.5 | 7.9 |
| 112757 OA Synovium9 | 1.7 | 0.0 |
| 112758 OA Synovial Fluid Cells9 | 6.7 | 20.4 |
| 117125 RA Cartilage Rep2 | 2.3 | 4.7 |
| 113492 Bone2 RA | 42.3 | 26.8 |
| 113493 Synovium2 RA | 16.6 | 3.7 |
| 113494 Syn Fluid Cells RA | 31.2 | 23.0 |
| 113499 Cartilage4 RA | 20.9 | 20.0 |
| 113500 Bone4 RA | 22.8 | 39.5 |
| 113501 Synovium4 RA | 26.6 | 24.8 |
| 113502 Syn Fluid Cells4 RA | 26.2 | 19.2 |
| 113495 Cartilage3 RA | 22.4 | 16.5 |
| 113496 Bone3 RA | 13.0 | 18.4 |
| 113497 Synovium3 RA | 7.0 | 9.9 |
| 113498 Syn Fluid Cells3 RA | 25.9 | 27.4 |
| 117106 Normal Cartilage Rep20 | 6.9 | 1.9 |
| 113663 Bone3 Normal | 0.0 | 0.0 |
| 113664 Synovium3 Normal | 0.0 | 0.0 |
| 113665 Syn Fluid Cells3 Normal | 1.2 | 0.0 |
| 117107 Normal Cartilage Rep22 | 12.9 | 0.0 |
| 113667 Bone4 Normal | 50.7 | 45.4 |
| 113668 Synovium4 Normal | 50.0 | 64.6 |
| 113669 Syn Fluid Cells4 Normal | 100.0 | 100.0 |

Column A - Rel. Exp. (%) Ag2695, Run 249247284
Column B - Rel. Exp. (%) Ag2695, Run 249259794

TABLE DC
CNS_neurodegeneration_v1.0

| Tissue Name | A | B |
|---|---|---|
| AD 1 Hippo | 20.4 | 10.6 |
| AD 2 Hippo | 51.4 | 39.0 |
| AD 3 Hippo | 16.0 | 9.4 |
| AD 4 Hippo | 17.9 | 10.7 |
| AD 5 hippo | 100.0 | 88.9 |
| AD 6 Hippo | 33.9 | 25.2 |
| Control 2 Hippo | 22.8 | 21.6 |
| Control 4 Hippo | 48.3 | 22.7 |
| Control (Path) 3 Hippo | 24.8 | 9.9 |
| AD 1 Temporal Ctx | 26.6 | 31.4 |
| AD 2 Temporal Ctx | 29.5 | 40.1 |
| AD 3 Temporal Ctx | 7.1 | 14.2 |
| AD 4 Temporal Ctx | 41.5 | 41.5 |
| AD 5 Inf Temporal Ctx | 88.3 | 100.0 |
| AD 5 Sup Temporal Ctx | 62.9 | 44.1 |
| AD 6 Inf Temporal Ctx | 35.1 | 26.4 |
| AD 6 Sup Temporal Ctx | 40.3 | 33.2 |
| Control 1 Temporal Ctx | 49.0 | 46.0 |
| Control 2 Temporal Ctx | 51.4 | 42.3 |
| Control 3 Temporal Ctx | 28.5 | 31.9 |
| Control 4 Temporal Ctx | 22.7 | 36.3 |
| Control (Path) 1 Temporal Ctx | 90.8 | 70.2 |
| Control (Path) 2 Temporal Ctx | 64.2 | 50.3 |
| Control (Path) 3 Temporal Ctx | 49.7 | 19.6 |
| Control (Path) 4 Temporal Ctx | 80.1 | 71.7 |
| AD 1 Occipital Ctx | 19.1 | 23.5 |
| AD 2 Occipital Ctx (Missing) | 0.0 | 0.0 |
| AD 3 Occipital Ctx | 17.4 | 8.7 |
| AD 4 Occipital Ctx | 51.8 | 33.7 |
| AD 5 Occipital Ctx | 14.5 | 22.5 |
| AD 6 Occipital Ctx | 59.0 | 44.4 |
| Control 1 Occipital Ctx | 43.2 | 34.9 |
| Control 2 Occipital Ctx | 80.1 | 57.0 |
| Control 3 Occipital Ctx | 46.3 | 37.6 |
| Control 4 Occipital Ctx | 37.1 | 11.2 |
| Control (Path) 1 Occipital Ctx | 87.7 | 85.3 |
| Control (Path) 2 Occipital Ctx | 50.3 | 31.0 |
| Control (Path) 3 Occipital Ctx | 36.6 | 22.2 |
| Control (Path) 4 Occipital Ctx | 84.7 | 71.7 |
| Control 1 Parietal Ctx | 60.7 | 48.6 |
| Control 2 Parietal Ctx | 55.5 | 40.9 |
| Control 3 Parietal Ctx | 22.8 | 10.9 |
| Control (Path) 1 Parietal Ctx | 97.3 | 58.2 |
| Control (Path) 2 Parietal Ctx | 76.3 | 46.3 |
| Control (Path) 3 Parietal Ctx | 25.0 | 28.5 |
| Control (Path) 4 Parietal Ctx | 84.7 | 72.2 |

Column A - Rel. Exp. (%) Ag2695, Run 209751330
Column B - Rel. Exp. (%) Ag2695, Run 219966626

TABLE DD
Panel 1.3D

| Tissue Name | A | B |
|---|---|---|
| Liver adenocarcinoma | 0.0 | 0.0 |
| Pancreas | 2.1 | 4.2 |
| Pancreatic ca. CAPAN 2 | 0.0 | 0.0 |
| Adrenal gland | 67.4 | 100.0 |
| Thyroid | 5.1 | 2.0 |
| Salivary gland | 5.1 | 3.5 |
| Pituitary gland | 1.0 | 4.9 |
| Brain (fetal) | 3.5 | 0.0 |
| Brain (whole) | 20.3 | 16.0 |
| Brain (amygdala) | 22.2 | 12.5 |
| Brain (cerebellum) | 2.1 | 4.5 |
| Brain (hippocampus) | 61.1 | 35.1 |
| Brain (substantia nigra) | 9.2 | 8.8 |
| Brain (thalamus) | 8.7 | 18.6 |
| Cerebral Cortex | 45.1 | 53.2 |
| Spinal cord | 11.3 | 8.0 |
| glio/astro U87-MG | 0.0 | 0.0 |
| glio/astro U-118-MG | 4.2 | 0.0 |
| astrocytoma SW1783 | 0.0 | 0.0 |
| neuro*; met SK-N-AS | 0.0 | 0.0 |
| astrocytoma SF-539 | 9.0 | 0.0 |
| astrocytoma SNB-75 | 0.0 | 0.0 |
| glioma SNB-19 | 0.0 | 2.9 |
| glioma U251 | 0.0 | 0.0 |
| glioma SF-295 | 0.0 | 2.0 |
| Heart (fetal) | 17.4 | 16.5 |
| Heart | 41.5 | 33.0 |
| Skeletal muscle (fetal) | 8.6 | 5.2 |
| Skeletal muscle | 0.0 | 0.0 |
| Bone marrow | 3.5 | 11.7 |
| Thymus | 1.4 | 6.8 |
| Spleen | 100.0 | 59.0 |
| Lymph node | 32.3 | 26.8 |
| Colorectal | 17.9 | 20.2 |
| Stomach | 9.5 | 0.0 |
| Small intestine | 13.4 | 39.0 |
| Colon ca. SW480 | 0.0 | 0.0 |
| Colon ca.* SW620 (SW480 met) | 0.0 | 0.0 |
| Colon ca. HT29 | 0.0 | 1.9 |
| Colon ca. HCT-116 | 0.0 | 0.0 |
| Colon ca. CaCo-2 | 0.0 | 0.0 |
| Colon ca. tissue (ODO3866) | 11.0 | 3.4 |
| Colon ca. HCC-2998 | 0.6 | 0.0 |
| Gastric ca.* (liver met) NCI-N87 | 0.0 | 1.9 |
| Bladder | 0.0 | 2.4 |
| Trachea | 4.2 | 0.0 |
| Kidney | 2.4 | 0.0 |
| Kidney (fetal) | 0.0 | 3.9 |
| Renal ca. 786-0 | 0.0 | 0.0 |
| Renal ca. A498 | 0.0 | 1.9 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 1.5 | 3.9 |
| Liver (fetal) | 0.0 | 2.5 |
| Liver ca. (hepatoblast) HepG2 | 0.0 | 0.0 |
| Lung | 18.8 | 16.2 |
| Lung (fetal) | 3.7 | 3.7 |
| Lung ca. (small cell) LX-1 | 0.0 | 0.0 |
| Lung ca. (small cell) NCI-H69 | 0.0 | 0.0 |
| Lung ca. (s. cell var.) SHP-77 | 0.0 | 0.0 |
| Lung ca. (large cell) NCI-H460 | 0.0 | 0.0 |
| Lung ca. (non-sm. cell) A549 | 0.0 | 0.0 |
| Lung ca. (non-s. cell) NCI-H23 | 0.0 | 1.8 |
| Lung ca. (non-s. cell) HOP-62 | 0.0 | 0.0 |
| Lung ca. (non-s. cl) NCI-H522 | 0.0 | 0.0 |
| Lung ca. (squam.) SW 900 | 0.0 | 0.0 |
| Lung ca. (squam.) NCI-H596 | 2.3 | 0.0 |
| Mammary gland | 5.2 | 9.0 |
| Breast ca.* (pl. ef) MCF-7 | 0.0 | 0.0 |
| Breast ca.* (pl. ef) MDA-MB-231 | 0.0 | 0.0 |
| Breast ca.* (pl. ef) T47D | 0.0 | 0.0 |
| Breast ca. BT-549 | 0.0 | 0.0 |
| Breast ca. MDA-N | 0.0 | 0.0 |
| Ovary | 16.2 | 9.1 |
| Ovarian ca. OVCAR-3 | 0.0 | 0.0 |
| Ovarian ca. OVCAR-4 | 0.0 | 0.0 |
| Ovarian ca. OVCAR-5 | 0.0 | 0.0 |
| Ovarian ca. OVCAR-8 | 0.0 | 0.0 |
| Ovarian ca. IGROV-1 | 0.0 | 0.0 |
| Ovarian ca.* (ascites) SK-OV-3 | 0.0 | 0.0 |
| Uterus | 6.5 | 4.3 |
| Placenta | 49.0 | 32.5 |
| Prostate | 15.0 | 2.2 |
| Prostate ca.* (bone met) PC-3 | 0.0 | 0.0 |
| Testis | 2.4 | 11.0 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 4.0 | 4.3 |
| Adipose | 15.5 | 16.7 |

Column A - Rel. Exp. (%) Ag2695, Run 153140732
Column B - Rel. Exp. (%) Ag2695, Run 153830081

TABLE DE
Panel 2D

| Tissue Name | A | B |
|---|---|---|
| Normal Colon | 3.8 | 3.3 |
| CC Well to Mod Diff (ODO3866) | 1.5 | 0.6 |
| CC Margin (ODO3866) | 2.7 | 0.5 |
| CC Gr.2 rectosigmoid (ODO3868) | 0.1 | 0.2 |
| CC Margin (ODO3868) | 0.9 | 0.3 |
| CC Mod Diff (ODO3920) | 0.6 | 0.2 |
| CC Margin (ODO3920) | 0.5 | 0.3 |
| CC Gr.2 ascend colon (ODO3921) | 3.2 | 2.2 |
| CC Margin (ODO3921) | 0.8 | 0.6 |
| CC from Partial Hepatectomy (ODO4309) Mets | 1.6 | 2.0 |
| Liver Margin (ODO4309) | 1.3 | 1.5 |
| Colon mets to lung (OD04451-01) | 0.3 | 1.2 |
| Lung Margin (OD04451-02) | 2.9 | 1.7 |
| Normal Prostate 6546-1 | 2.0 | 0.8 |
| Prostate Cancer (OD04410) | 1.9 | 1.7 |
| Prostate Margin (OD04410) | 3.5 | 3.9 |
| Prostate Cancer (OD04720-01) | 1.7 | 0.5 |
| Prostate Margin (OD04720-02) | 5.0 | 4.4 |
| Normal Lung 061010 | 6.9 | 7.8 |
| Lung Met to Muscle (ODO4286) | 2.2 | 2.0 |
| Muscle Margin (ODO4286) | 0.3 | 0.5 |
| Lung Malignant Cancer (OD03126) | 2.4 | 3.7 |
| Lung Margin (OD03126) | 6.9 | 6.7 |
| Lung Cancer (OD04404) | 5.5 | 2.1 |
| Lung Margin (OD04404) | 1.6 | 1.9 |
| Lung Cancer (OD04565) | 0.6 | 0.5 |
| Lung Margin (OD04565) | 1.0 | 2.0 |
| Lung Cancer (OD04237-01) | 4.5 | 3.8 |
| Lung Margin (OD04237-02) | 3.9 | 5.2 |
| Ocular Mel Met to Liver (ODO4310) | 0.0 | 0.2 |
| Liver Margin (ODO4310) | 1.7 | 0.7 |
| Melanoma Mets to Lung (OD04321) | 100.0 | 100.0 |
| Lung Margin (OD04321) | 7.3 | 6.5 |
| Normal Kidney | 6.4 | 5.0 |
| Kidney Ca, Nuclear grade 2 (OD04338) | 34.2 | 33.9 |
| Kidney Margin (OD04338) | 5.2 | 5.6 |
| Kidney Ca Nuclear grade 1/2 (OD04339) | 1.4 | 0.8 |
| Kidney Margin (OD04339) | 0.9 | 2.0 |
| Kidney Ca, Clear cell type (OD04340) | 8.5 | 10.3 |
| Kidney Margin (OD04340) | 11.3 | 6.6 |
| Kidney Ca, Nuclear grade 3 (OD04348) | 1.4 | 1.9 |
| Kidney Margin (OD04348) | 5.4 | 7.3 |
| Kidney Cancer (OD04622-01) | 49.0 | 63.7 |
| Kidney Margin (OD04622-03) | 0.7 | 1.2 |
| Kidney Cancer (OD04450-01) | 1.4 | 1.4 |
| Kidney Margin (OD04450-03) | 5.7 | 4.6 |
| Kidney Cancer 8120607 | 0.2 | 0.0 |
| Kidney Margin 8120608 | 1.4 | 0.5 |
| Kidney Cancer 8120613 | 0.1 | 0.1 |
| Kidney Margin 8120614 | 0.6 | 0.0 |
| Kidney Cancer 9010320 | 4.0 | 2.5 |
| Kidney Margin 9010321 | 0.4 | 1.1 |
| Normal Uterus | 0.4 | 0.3 |
| Uterus Cancer 064011 | 1.2 | 0.9 |
| Normal Thyroid | 0.6 | 0.8 |
| Thyroid Cancer 064010 | 7.3 | 5.7 |
| Thyroid Cancer A302152 | 7.1 | 8.9 |
| Thyroid Margin A302153 | 1.3 | 1.1 |
| Normal Breast | 1.1 | 1.8 |
| Breast Cancer (OD04566) | 0.2 | 1.3 |
| Breast Cancer (OD04590-01) | 2.2 | 2.0 |
| Breast Cancer Mets (OD04590-03) | 6.7 | 8.5 |
| Breast Cancer Metastasis (OD04655-05) | 2.7 | 2.7 |
| Breast Cancer 064006 | 0.6 | 0.6 |
| Breast Cancer 1024 | 0.7 | 0.6 |
| Breast Cancer 9100266 | 0.1 | 0.3 |
| Breast Margin 9100265 | 0.2 | 0.5 |
| Breast Cancer A209073 | 2.3 | 1.7 |
| Breast Margin A209073 | 0.4 | 0.3 |
| Normal Liver | 0.6 | 0.2 |
| Liver Cancer 064003 | 0.8 | 0.0 |
| Liver Cancer 1025 | 0.2 | 0.2 |
| Liver Cancer 1026 | 0.8 | 0.6 |
| Liver Cancer 6004-T | 0.4 | 0.5 |
| Liver Tissue 6004-N | 0.9 | 0.3 |
| Liver Cancer 6005-T | 0.2 | 0.2 |
| Liver Tissue 6005-N | 0.3 | 0.3 |
| Normal Bladder | 0.9 | 1.5 |
| Bladder Cancer 1023 | 0.1 | 0.4 |
| Bladder Cancer A302173 | 0.5 | 1.2 |
| Bladder Cancer (OD04718-01) | 1.8 | 1.2 |
| Bladder Normal Adjacent (OD04718-03) | 2.2 | 1.9 |
| Normal Ovary | 0.6 | 0.2 |
| Ovarian Cancer 064008 | 2.9 | 1.7 |
| Ovarian Cancer (OD04768-07) | 14.4 | 10.8 |
| Ovary Margin (OD04768-08) | 1.4 | 1.0 |
| Normal Stomach | 0.6 | 1.5 |
| Gastric Cancer 9060358 | 0.5 | 0.7 |
| Stomach Margin 9060359 | 0.6 | 0.6 |
| Gastric Cancer 9060395 | 1.1 | 1.2 |
| Stomach Margin 9060394 | 1.2 | 0.4 |
| Gastric Cancer 9060397 | 0.9 | 0.9 |
| Stomach Margin 9060396 | 1.7 | 1.1 |
| Gastric Cancer 064005 | 3.0 | 2.6 |

Column A - Rel. Exp. (%) Ag2695, Run 153140795
Column B - Rel. Exp. (%) Ag2695, Run 153789847

TABLE DF
Panel 4D

| Tissue Name | A | B |
|---|---|---|
| Secondary Th1 act | 1.0 | 1.6 |
| Secondary Th2 act | 21.0 | 29.5 |
| Secondary Tr1 act | 5.0 | 10.8 |
| Secondary Th1 rest | 0.6 | 3.8 |
| Secondary Th2 rest | 13.9 | 15.4 |
| Secondary Tr1 rest | 6.1 | 5.0 |
| Primary Th1 act | 0.0 | 2.6 |
| Primary Th2 act | 39.0 | 54.3 |
| Primary Tr1 act | 10.7 | 21.6 |
| Primary Th1 rest | 21.0 | 24.7 |
| Primary Th2 rest | 26.1 | 21.2 |
| Primary Tr1 rest | 11.7 | 19.1 |
| CD45RA CD4 lymphocyte act | 9.6 | 7.1 |
| CD45RO CD4 lymphocyte act | 15.6 | 17.3 |
| CD8 lymphocyte act | 11.7 | 9.3 |
| Secondary CD8 lymphocyte rest | 8.4 | 9.0 |
| Secondary CD8 lymphocyte act | 22.2 | 18.3 |
| CD4 lymphocyte none | 18.6 | 9.1 |
| 2ry Th1/Th2/Tr1_anti-CD95 CH11 | 3.8 | 7.6 |
| LAK cells rest | 43.2 | 32.5 |
| LAK cells IL-2 | 15.9 | 18.0 |
| LAK cells IL-2 + IL-12 | 32.3 | 30.8 |
| LAK cells IL-2 + IFN gamma | 62.9 | 57.8 |
| LAK cells IL-2 + IL-18 | 39.2 | 52.1 |
| LAK cells PMA/ionomycin | 43.5 | 52.9 |
| NK Cells IL-2 rest | 37.4 | 35.6 |
| Two Way MLR 3 day | 26.1 | 23.0 |
| Two Way MLR 5 day | 17.0 | 10.7 |
| Two Way MLR 7 day | 2.1 | 7.4 |
| PBMC rest | 19.9 | 28.3 |
| PBMC PWM | 28.3 | 31.4 |
| PBMC PHA-L | 10.9 | 14.4 |
| Ramos (B cell) none | 0.0 | 0.1 |
| Ramos (B cell) ionomycin | 0.0 | 0.0 |
| B lymphocytes PWM | 9.0 | 9.5 |
| B lymphocytes CD40L and IL-4 | 9.5 | 11.1 |
| EOL-1 dbcAMP | 1.1 | 0.0 |
| EOL-1 dbcAMP PMA/ionomycin | 3.7 | 0.6 |
| Dendritic cells none | 3.7 | 10.3 |
| Dendritic cells LPS | 6.1 | 8.1 |
| Dendritic cells anti-CD40 | 4.9 | 3.6 |
| Monocytes rest | 100.0 | 100.0 |
| Monocytes LPS | 0.0 | 0.3 |
| Macrophages rest | 0.6 | 3.7 |
| Macrophages LPS | 1.0 | 3.1 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.0 | 0.0 |
| Lung Microvascular EC none | 0.0 | 0.0 |
| Lung Microvascular EC TNFalpha + IL-1beta | 0.0 | 0.0 |
| Microvascular Dermal EC none | 0.0 | 0.0 |
| Microsvasular Dermal EC TNFalpha + IL-1beta | 0.0 | 0.0 |
| Bronchial epithelium TNFalpha + IL1beta | 0.0 | 0.0 |
| Small airway epithelium none | 0.0 | 0.0 |
| Small airway epithelium TNFalpha + IL-1beta | 0.0 | 0.0 |
| Coronery artery SMC rest | 0.0 | 0.0 |
| Coronery artery SMC TNFalpha + IL-1beta | 0.0 | 0.0 |
| Astrocytes rest | 0.0 | 0.5 |
| Astrocytes TNFalpha + IL-1beta | 0.0 | 0.0 |
| KU-812 (Basophil) rest | 0.5 | 2.4 |
| KU-812 (Basophil) PMA/ionomycin | 2.9 | 1.7 |
| CCD1106 (Keratinocytes) none | 0.0 | 0.7 |
| CCD1106 (Keratinocytes) TNFalpha + IL-1beta | 0.0 | 0.0 |
| Liver cirrhosis | 4.8 | 6.3 |
| Lupus kidney | 0.6 | 0.0 |
| NCI-H292 none | 0.0 | 0.0 |
| NCI-H292 IL-4 | 0.0 | 0.0 |
| NCI-H292 IL-9 | 0.0 | 0.0 |
| NCI-H292 IL-13 | 0.0 | 0.0 |
| NCI-H292 IFN gamma | 0.0 | 0.0 |
| HPAEC none | 0.0 | 0.5 |
| HPAEC TNF alpha + IL-1 beta | 0.0 | 0.0 |
| Lung fibroblast none | 0.0 | 0.0 |
| Lung fibroblast TNF alpha + IL-1 beta | 0.0 | 0.0 |
| Lung fibroblast IL-4 | 0.0 | 0.0 |
| Lung fibroblast IL-9 | 0.0 | 0.0 |
| Lung fibroblast IL-13 | 0.0 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 17.1 | 20.3 |
| Dermal fibroblast CCD1070 IL-1 beta | 0.0 | 0.0 |
| Dermal fibroblast IFN gamma | 0.0 | 0.0 |
| Dermal fibroblast IL-4 | 1.1 | 0.0 |
| IBD Colitis 2 | 1.1 | 0.0 |
| IBD Crohn's | 2.2 | 3.5 |
| Colon | 21.5 | 15.6 |
| Lung | 17.6 | 14.4 |
| Thymus | 11.8 | 7.1 |
| Kidney | 33.7 | 20.3 |

Column A - Rel. Exp. (%) Ag2695, Run 153140809
Column B - Rel. Exp. (%) Ag2695, Run 153766369

AI_comprehensive panel_v1.0 Summary: Ag2695 Two experiments with same probe-primer sets are in good agreement. Low expression of this gene is mainly seen in normal synovium and synovial fluid cells. Low expression of this gene is also seen in osteoarthritis bone, cartilage, synovium, RA bone, normal lung and a psoriasis sample.

CNS_neurodegeneration_v1.0 Summary: Data obtained from RTQPCR assays performed for this panel was analyized using multivariate analysis (ANOVA). The multivariate results obtained using Ag2695 in two experiments with same probe-primer sets are in good agreement. This gene is found to be down-regulated in the temporal cortex of Alzheimer's disease patients. Therefore, up-regulation of this gene or its gene product, or treatment with specific agonists for this protein encoded by this gene may be of use in reversing the dementia/memory loss associated with this disease and neuronal death.

Panel 1.3D Summary: Ag2695 Two experiments with same probe-primer sets are in good agreement. Highest expression of this gene is seen in adrenal gland and spleen (CTS=31.7). Significant expression of this gene is seen mainly in the normal tissues including brain, lymph node, heart, gastrointestinal tract, lung, ovary, placenta and adipose tissue. Interestingly, expression of this gene is low or undetectable in any of the cancer cell lines. Therefore, therapeutic modulation of this gene or its protein product may be useful in the treatment of cancer, metabolic and CNS disorders.

Panel 2D Summary: Ag2695 Two experiments with same probe-primer sets are in good agreement. Highest expression of this gene is detected in metastatic melanoma (CTs=26-27.8). High to moderate expression of this gene is also seen in normal and cancer samples from colon, lung, prostate, liver, prostate, thyroid, uterus, breast, ovary and stomach. Interestingly, expression of this gene is upregulated in ovarian, thyroid and kidney cancers compared to corresponding normal adjacent normal tissues. Therefore, therapeutic modulation of this gene or its protein product may be useful in the treatment of ovarian, thyroid and kidney cancers.

Panel 4D Summary: Ag2695 Two experiments with same probe-primer sets are in good agreement. Highest expression of this gene is seen in resting monocytes (CT=29.6). This gene is expressed by T lymphocytes prepared under a number of conditions at moderate levels and is expressed at significant levels in treated and untreated dendritic cells, LAK cells, PBMC, activated B lymphocytes, activated dermal fibroblasts, liver cirrhosis sample and normal tissues represented by colon, lung, thymus and kidney. Dendritic cells are powerful antigen-presenting cells (APC) whose function is pivotal in the initiation and maintenance of normal immune responses. Autoimmunity and inflammation may also be reduced by suppression of this function. Therefore, small molecule drugs or the antibodies that antagonize the function of this gene or its protein product may reduce or eliminate the symptoms in patients with several types of autoimmune and inflammatory diseases, such as lupus erythematosus, Crohn's disease, ulcerative colitis, multiple sclerosis, chronic obstructive pulmonary disease, asthma, emphysema, rheumatoid arthritis, or psoriasis.

E. CG54566-01: Serotonin Receptor

Expression of gene CG54566-01 was assessed using the primer-probe set Ag1252, described in Table EA. Results of the RTQ-PCR runs are shown in Tables EB, EC, ED, EE, EF, EG and EH.

TABLE EA

Probe Name Ag1252

| | | | | |
|---|---|---|---|---|
| | | | Start | SEQ ID |
| Primers | Sequences | Length | Position | No |

| | | | | |
|---|---|---|---|---|
| Forward | 5′-ccagtgccagtaccagaatatc-3′ | 22 | 1210 | 110 |
| Probe | TET-5′-aaccagacactctcagctgcaggcat-3′-TAMRA | 26 | 1232 | 111 |
| Reverse | 5′-ctctctggcctctcagcaa-3′ | 19 | 1275 | 112 |

TABLE EB
AI_comprehensive panel_v1.0

| Tissue Name | A |
|---|---|
| 110967 COPD-F | 0.0 |
| 110980 COPD-F | 1.3 |
| 110968 COPD-M | 5.5 |
| 110977 COPD-M | 14.9 |
| 110989 Emphysema-F | 74.7 |
| 110992 Emphysema-F | 2.0 |
| 110993 Emphysema-F | 24.5 |
| 110994 Emphysema-F | 10.0 |
| 110995 Emphysema-F | 33.9 |
| 110996 Emphysema-F | 0.0 |
| 110997 Asthma-M | 0.8 |
| 111001 Asthma-F | 3.3 |
| 111002 Asthma-F | 31.2 |
| 111003 Atopic Asthma-F | 32.8 |
| 111004 Atopic Asthma-F | 34.4 |
| 111005 Atopic Asthma-F | 30.1 |
| 111006 Atopic Asthma-F | 11.9 |
| 111417 Allergy-M | 21.0 |
| 112347 Allergy-M | 0.9 |
| 112349 Normal Lung-F | 1.3 |
| 112357 Normal Lung-F | 62.4 |
| 112354 Normal Lung-M | 28.3 |
| 112374 Crohns-F | 19.8 |
| 112389 Match Control Crohns-F | 9.3 |
| 112375 Crohns-F | 25.0 |
| 112732 Match Control Crohns-F | 16.0 |
| 112725 Crohns-M | 2.8 |
| 112387 Match Control Crohns-M | 8.3 |
| 112378 Crohns-M | 2.6 |
| 112390 Match Control Crohns-M | 56.6 |
| 112726 Crohns-M | 37.4 |
| 112731 Match Control Crohns-M | 24.3 |
| 112380 Ulcer Col-F | 37.6 |
| 112734 Match Control Ulcer Col-F | 35.4 |
| 112384 Ulcer Col-F | 42.3 |
| 112737 Match Control Ulcer Col-F | 11.2 |
| 112386 Ulcer Col-F | 10.4 |
| 112738 Match Control Ulcer Col-F | 2.8 |
| 112381 Ulcer Col-M | 3.6 |
| 112735 Match Control Ulcer Col-M | 8.7 |
| 112382 Ulcer Col-M | 13.9 |
| 112394 Match Control Ulcer Col-M | 7.6 |
| 112383 Ulcer Col-M | 36.3 |
| 112736 Match Control Ulcer Col-M | 11.2 |
| 112423 Psoriasis-F | 29.1 |
| 112427 Match Control Psoriasis-F | 100.0 |
| 112418 Psoriasis-M | 16.2 |
| 112723 Match Control Psoriasis-M | 39.2 |
| 112419 Psoriasis-M | 37.9 |
| 112424 Match Control Psoriasis-M | 19.3 |
| 112420 Psoriasis-M | 65.1 |
| 112425 Match Control Psoriasis-M | 95.9 |
| 104689 (MF) OA Bone-Backus | 80.7 |
| 104690 (MF) Adj “Normal” Bone-Backus | 58.2 |
| 104691 (MF) OA Synovium-Backus | 73.2 |
| 104692 (BA) OA Cartilage-Backus | 27.2 |
| 104694 (BA) OA Bone-Backus | 45.1 |
| 104695 (BA) Adj “Normal” Bone-Backus | 44.4 |
| 104696 (BA) OA Synovium-Backus | 51.8 |
| 104700 (SS) OA Bone-Backus | 22.4 |
| 104701 (SS) Adj “Normal” Bone-Backus | 33.4 |
| 104702 (SS) OA Synovium-Backus | 78.5 |
| 117093 OA Cartilage Rep7 | 18.2 |
| 112672 OA Bone5 | 47.0 |
| 112673 OA Synovium5 | 17.8 |
| 112674 OA Synovial Fluid cells5 | 15.9 |
| 117100 OA Cartilage Rep 14 | 6.3 |
| 112756 OA Bone9 | 23.2 |
| 112757 OA Synovium9 | 7.7 |
| 112758 OA Synovial Fluid Cells9 | 15.7 |
| 117125 RA Cartilage Rep2 | 39.5 |
| 113492 Bone2 RA | 28.5 |
| 113493 Synovium2 RA | 8.4 |
| 113494 Syn Fluid Cells RA | 15.8 |
| 113499 Cartilage4 RA | 17.7 |
| 113500 Bone4 RA | 37.9 |
| 113501 Synovium4 RA | 15.2 |
| 113502 Syn Fluid Cells4 RA | 15.3 |
| 113495 Cartilage3 RA | 12.8 |
| 113496 Bone3 RA | 29.5 |
| 113497 Synovium3 RA | 8.9 |
| 113498 Syn Fluid Cells3 RA | 24.5 |
| 117106 Normal Cartilage Rep20 | 2.1 |
| 113663 Bone3 Normal | 3.4 |
| 113664 Synovium3 Normal | 0.0 |
| 113665 Syn Fluid Cells3 Normal | 0.0 |
| 117107 Normal Cartilage Rep22 | 9.9 |
| 113667 Bone4 Normal | 18.4 |
| 113668 Synovium4 Normal | 15.6 |
| 113669 Syn Fluid Cells4 Normal | 26.8 |

Column A - Rel. Exp. (%) Ag1252, Run 249259629

TABLE EC
CNS_neurodegeneration_v1.0

| Tissue Name | A | B |
|---|---|---|
| AD 1 Hippo | 10.0 | 6.8 |
| AD 2 Hippo | 28.1 | 21.5 |
| AD 3 Hippo | 5.9 | 5.1 |
| AD 4 Hippo | 11.6 | 6.1 |
| AD 5 Hippo | 67.4 | 80.7 |
| AD 6 Hippo | 55.5 | 22.8 |
| Control 2 Hippo | 47.3 | 38.4 |
| Control 4 Hippo | 0.0 | 8.7 |
| Control (Path) 3 Hippo | 1.0 | 2.4 |
| AD 1 Temporal Ctx | 6.6 | 1.4 |
| AD 2 Temporal Ctx | 20.0 | 13.8 |
| AD 3 Temporal Ctx | 2.8 | 0.8 |
| AD 4 Temporal Ctx | 15.7 | 10.9 |
| AD 5 Inf Temporal Ctx | 56.3 | 100.0 |
| AD 5 Sup Temporal Ctx | 30.4 | 62.4 |
| AD 6 Inf Temporal Ctx | 33.9 | 20.0 |
| AD 6 Sup Temporal Ctx | 33.4 | 24.7 |
| Control 1 Temporal Ctx | 5.8 | 4.8 |
| Control 2 Temporal Ctx | 77.9 | 46.0 |
| Control 3 Temporal Ctx | 17.3 | 18.3 |
| Control 3 Temporal Ctx | 8.5 | 3.9 |
| Control (Path) 1 Temporal Ctx | 74.7 | 47.0 |
| Control (Path) 2 Temporal Ctx | 70.7 | 21.0 |
| Control (Path) 3 Temporal Ctx | 2.0 | 1.9 |
| Control (Path) 4 Temporal Ctx | 39.8 | 23.5 |
| AD 1 Occipital Ctx | 1.5 | 2.5 |
| AD 2 Occipital Ctx (Missing) | 0.0 | 0.0 |
| AD 3 Occipital Ctx | 1.3 | 1.7 |
| AD 4 Occipital Ctx | 14.6 | 11.0 |
| AD 5 Occipital Ctx | 59.9 | 77.9 |
| AD 6 Occipital Ctx | 11.2 | 20.4 |
| Control 1 Occipital Ctx | 2.5 | 1.8 |
| Control 2 Occipital Ctx | 54.3 | 45.1 |
| Control 3 Occipital Ctx | 18.4 | 7.3 |
| Control 4 Occipital Ctx | 6.3 | 2.0 |
| Control (Path) 1 Occipital Ctx | 100.0 | 63.3 |
| Control (Path) 2 Occipital Ctx | 11.1 | 5.2 |
| Control (Path) 3 Occipital Ctx | 3.8 | 4.8 |
| Control (Path) 4 Occipital Ctx | 12.9 | 13.5 |
| Control 1 Parietal Ctx | 4.8 | 1.3 |
| Control 2 Parietal Ctx | 29.3 | 94.0 |
| Control 3 Parietal Ctx | 18.8 | 16.0 |
| Control (Path) 1 Parietal Ctx | 100.0 | 83.5 |
| Control (Path) 2 Parietal Ctx | 23.5 | 16.7 |
| Control (Path) 3 Parietal Ctx | 3.2 | 2.0 |
| Control (Path) 4 Parietal Ctx | 59.5 | 30.8 |

Column A - Rel. Exp. (%) Ag1252, Run 206228025
Column B - Rel. Exp. (%) Ag1252, Run 219923396

TABLE ED
General_screening_panel_v1.4

| Tissue Name | A |
|---|---|
| Adipose | 11.8 |
| Melanoma* Hs688(A).T | 19.5 |
| Melanoma* Hs688(B).T | 33.0 |
| Melanoma* M14 | 19.8 |
| Melanoma* LOXIMVI | 35.4 |
| Melanoma* SK-MEL-5 | 6.3 |
| Squamous cell carcinoma SCC-4 | 24.1 |
| Testis Pool | 9.2 |
| Prostate ca.* (bone met) PC-3 | 11.2 |
| Prostate Pool | 7.6 |
| Placenta | 8.1 |
| Uterus Pool | 3.0 |
| Ovarian ca. OVCAR-3 | 12.0 |
| Ovarian ca. SK-OV-3 | 15.4 |
| Ovarian ca. OVCAR-4 | 3.8 |
| Ovarian ca. OVCAR-5 | 21.6 |
| Ovarian ca. IGROV-1 | 16.5 |
| Ovarian ca. OVCAR-8 | 9.5 |
| Ovary | 18.3 |
| Breast ca. MCF-7 | 84.1 |
| Breast ca. MDA-MB-231 | 23.5 |
| Breast ca. BT 549 | 34.6 |
| Breast ca. T47D | 59.5 |
| Breast ca. MDA-N | 7.3 |
| Breast Pool | 9.5 |
| Trachea | 4.8 |
| Lung | 3.3 |
| Fetal Lung | 10.2 |
| Lung ca. NCI-N417 | 1.3 |
| Lung ca. LX-1 | 8.2 |
| Lung ca. NCI-H146 | 38.2 |
| Lung ca. SHP-77 | 2.3 |
| Lung ca. A549 | 15.1 |
| Lung ca. NCI-H526 | 1.2 |
| Lung ca. NCI-H23 | 18.9 |
| Lung ca. NCI-H460 | 11.9 |
| Lung ca. HOP-62 | 29.9 |
| Lung ca. NCI-H522 | 40.6 |
| Liver | 0.3 |
| Fetal Liver | 12.5 |
| Liver ca. HepG2 | 4.4 |
| Kidney Pool | 16.7 |
| Fetal Kidney | 11.6 |
| Renal ca. 786-0 | 15.9 |
| Renal ca. A498 | 1.2 |
| Renal ca. ACHN | 19.2 |
| Renal ca. UO-31 | 32.1 |
| Renal ca. TK-10 | 6.0 |
| Bladder | 4.8 |
| Gastric ca. (liver met.) NCI-N87 | 20.0 |
| Gastric ca. KATO III | 0.2 |
| Colon ca. SW-948 | 12.7 |
| Colon ca. SW480 | 32.8 |
| Colon ca.* (SW480 met) SW620 | 7.9 |
| Colon ca. HT29 | 1.3 |
| Colon ca. HCT-116 | 49.0 |
| Colon ca. CaCo-2 | 9.3 |
| Colon cancer tissue | 10.7 |
| Colon ca. SW1116 | 4.5 |
| Colon ca. Colo-205 | 0.8 |
| Colon ca. SW-48 | 1.3 |
| Colon Pool | 10.6 |
| Small Intestine Pool | 9.8 |
| Stomach Pool | 3.1 |
| Bone Marrow Pool | 2.6 |
| Fetal Heart | 2.5 |
| Heart Pool | 4.2 |
| Lymph Node Pool | 10.2 |
| Fetal Skeletal Muscle | 1.8 |
| Skeletal Muscle Pool | 2.0 |
| Spleen Pool | 8.0 |
| Thymus Pool | 18.0 |
| CNS cancer (glio/astro) U87-MG | 67.4 |
| CNS cancer (glio/astro) U-118-MG | 38.4 |
| CNS cancer (neuro; met) SK-N-AS | 11.4 |
| CNS cancer (astro) SF-539 | 10.6 |
| CNS cancer (astro) SNB-75 | 20.3 |
| CNS cancer (glio) SNB-19 | 19.6 |
| CNS cancer (glio) SF-295 | 67.4 |
| Brain (Amygdala) Pool | 10.1 |
| Brain (cerebellum) | 100.0 |
| Brain (fetal) | 17.2 |
| Brain (Hippocampus) Pool | 14.0 |
| Cerebral Cortex Pool | 20.9 |
| Brain (Substantia nigra) Pool | 16.7 |
| Brain (Thalamus) Pool | 20.4 |
| Brain (whole) | 22.7 |
| Spinal Cord Pool | 5.4 |
| Adrenal Gland | 10.9 |
| Pituitary gland Pool | 3.5 |
| Salivary Gland | 2.9 |
| Thyroid (female) | 8.4 |
| Pancreatic ca. CAPAN2 | 11.1 |
| Pancreas Pool | 10.0 |

Column A - Rel. Exp. (%) Ag1252, Run 212704837

TABLE EE
Panel 1.2

| Tissue Name | A |
|---|---|
| Endothelial cells | 27.7 |
| Heart (Fetal) | 1.5 |
| Pancreas | 7.9 |
| Pancreatic ca. CAPAN 2 | 2.9 |
| Adrenal Gland | 8.3 |
| Thyroid | 15.7 |
| Salivary gland | 4.7 |
| Pituitary gland | 8.5 |
| Brain (fetal) | 6.0 |
| Brain (whole) | 17.2 |
| Brain (amygdala) | 10.0 |
| Brain (cerebellum) | 4.8 |
| Brain (hippocampus) | 12.0 |
| Brain (thalamus) | 12.2 |
| Cerebral Cortex | 15.7 |
| Spinal cord | 2.8 |
| glio/astro U87-MG | 12.2 |
| glio/astro U-118-MG | 4.5 |
| astrocytoma SW1783 | 1.9 |
| neuro*; met SK-N-AS | 4.2 |
| astrocytoma SF-539 | 2.8 |
| astrocytoma SNB-75 | 0.8 |
| glioma SNB-19 | 11.0 |
| glioma U251 | 6.9 |
| glioma SF-295 | 10.0 |
| Heart | 5.9 |
| Skeletal Muscle | 2.9 |
| Bone marrow | 2.4 |
| Thymus | 1.5 |
| Spleen | 2.9 |
| Lymph node | 4.3 |
| Colorectal Tissue | 1.1 |
| Stomach | 3.3 |
| Small intestine | 3.7 |
| Colon ca. SW480 | 4.5 |
| Colon ca.* SW620 (SW480 met) | 5.2 |
| Colon ca. HT29 | 0.2 |
| Colon ca. HCT-116 | 8.0 |
| Colon ca. CaCo-2 | 4.1 |
| Colon ca. Tissue (ODO3866) | 1.2 |
| Colon ca. HCC-2998 | 8.1 |
| Gastric ca.* (liver met) NCI-N87 | 6.9 |
| Bladder | 12.0 |
| Trachea | 2.6 |
| Kidney | 8.4 |
| Kidney (fetal) | 9.2 |
| Renal ca. 786-0 | 3.7 |
| Renal ca. A498 | 1.1 |
| Renal ca. RXF 393 | 0.9 |
| Renal ca. ACHN | 10.4 |
| Renal ca. UO-31 | 11.0 |
| Renal ca. TK-10 | 3.9 |
| Liver | 7.2 |
| Liver (fetal) | 3.7 |
| Liver ca. (hepatoblast) HepG2 | 3.4 |
| Lung | 2.7 |
| Lung (fetal) | 3.1 |
| Lung ca. (small cell) LX-1 | 4.3 |
| Lung ca. (small cell) NCI-H69 | 1.8 |
| Lung ca. (s. cell var.) SHP-77 | 0.5 |
| Lung ca. (large cell)NCI-H460 | 27.4 |
| Lung ca. (non-sm. cell) A549 | 6.4 |
| Lung ca. (non-s. cell) NCI-H23 | 5.9 |
| Lung ca. (non-s. cell) HOP-62 | 26.4 |
| Lung ca. (non-s. cl) NCI-H522 | 100.0 |
| Lung ca. (squam.) SW 900 | 7.6 |
| Lung ca. (squam.) NCI-H596 | 2.4 |
| Mammary gland | 11.2 |
| Breast ca.* (pl. ef) MCF-7 | 15.6 |
| Breast ca.* (pl. ef) MDA-MB-231 | 2.3 |
| Breast ca.* (pl. ef) T47D | 2.1 |
| Breast ca. BT-549 | 6.5 |
| Breast ca. MDA-N | 0.0 |
| Ovary | 6.0 |
| Ovarian ca. OVCAR-3 | 7.3 |
| Ovarian ca. OVCAR-4 | 3.0 |
| Ovarian ca. OVCAR-5 | 14.1 |
| Ovarian ca. OVCAR-8 | 3.6 |
| Ovarian ca. IGROV-1 | 10.4 |
| Ovarian ca. (ascites) SK-OV-3 | 6.7 |
| Uterus | 3.5 |
| Placenta | 13.4 |
| Prostate | 5.9 |
| Prostate ca.* (bone met) PC-3 | 12.8 |
| Testis | 7.4 |
| Melanoma Hs688(A).T | 4.3 |
| Melanoma* (met) Hs688(B).T | 3.8 |
| Melanoma UACC-62 | 11.7 |
| Melanoma M14 | 3.6 |
| Melanoma LOX IMVI | 6.0 |
| Melanoma* (met) SK-MEL-5 | 4.5 |

Column A - Rel. Exp. (%) Ag1252, Run 129179690

TABLE EF
Panel 2D

| Tissue Name | A |
|---|---|
| Normal Colon | 21.0 |
| CC Well to Mod Diff (ODO3866) | 2.6 |
| CC Margin (ODO3866) | 2.3 |
| CC Gr.2 rectosigmoid (ODO3868) | 2.3 |
| CC Margin (ODO3868) | 0.2 |
| CC Mod Diff (ODO3920) | 7.7 |
| CC Margin (ODO3920) | 4.2 |
| CC Gr.2 ascend colon (ODO3921) | 7.3 |
| CC Margin (ODO3921) | 3.6 |
| CC from Partial Hepatectomy (ODO4309) Mets | 9.1 |
| Liver Margin (ODO4309) | 9.4 |
| Colon mets to lung (OD04451-01) | 7.0 |
| Lung Margin (OD04451-02) | 7.5 |
| Normal Prostate 6546-1 | 44.1 |
| Prostate Cancer (OD04410) | 32.5 |
| Prostate Margin (OD04410) | 18.7 |
| Prostate Cancer (OD04720-01) | 21.8 |
| Prostate Margin (OD04720-02) | 31.2 |
| Normal Lung 061010 | 9.9 |
| Lung Met to Muscle (ODO4286) | 8.1 |
| Muscle Margin (ODO4286) | 7.3 |
| Lung Malignant Cancer (OD03126) | 9.2 |
| Lung Margin (OD03126) | 14.5 |
| Lung Cancer (OD04404) | 14.5 |
| Lung Margin (OD04404) | 7.9 |
| Lung Cancer (OD04565) | 13.5 |
| Lung Margin (OD04565) | 7.9 |
| Lung Cancer (OD04237-01) | 15.8 |
| Lung Margin (OD04237-02) | 11.3 |
| Ocular Mel Met to Liver (ODO4310) | 6.7 |
| Liver Margin (ODO4310) | 6.5 |
| Melanoma Mets to Lung (OD04321) | 4.1 |
| Lung Margin (OD04321) | 8.1 |
| Normal Kidney | 27.5 |
| Kidney Ca, Nuclear grade 2 (OD04338) | 17.9 |
| Kidney Margin (OD04338) | 9.9 |
| Kidney Ca Nuclear grade 1/2 (OD04339) | 13.3 |
| Kidney Margin (OD04339) | 11.8 |
| Kidney Ca, Clear cell type (OD04340) | 23.2 |
| Kidney Margin (OD04340) | 14.8 |
| Kidney Ca, Nuclear grade 3 (OD04348) | 24.3 |
| Kidney Margin (OD04348) | 13.7 |
| Kidney Cancer (OD04622-01) | 9.8 |
| Kidney Margin (OD04622-03) | 2.0 |
| Kidney Cancer (OD04450-01) | 11.7 |
| Kidney Margin (OD04450-03) | 20.6 |
| Kidney Cancer 8120607 | 1.1 |
| Kidney Margin 8120608 | 2.2 |
| Kidney Cancer 8120613 | 10.7 |
| Kidney Margin 8120614 | 2.9 |
| Kidney Cancer 9010320 | 16.0 |
| Kidney Margin 9010321 | 8.5 |
| Normal Uterus | 3.0 |
| Uterus Cancer 064011 | 14.5 |
| Normal Thyroid | 20.2 |
| Thyroid Cancer 064010 | 8.9 |
| Thyroid Cancer A302152 | 11.0 |
| Thyroid Margin A302153 | 21.0 |
| Normal Breast | 22.5 |
| Breast Cancer (OD04566) | 40.1 |
| Breast Cancer (OD04590-01) | 20.4 |
| Breast Cancer Mets (OD04590-03) | 26.1 |
| Breast Cancer Metastasis (OD04655-05) | 100.0 |
| Breast Cancer 064006 | 10.7 |
| Breast Cancer 1024 | 15.7 |
| Breast Cancer 9100266 | 14.8 |
| Breast Margin 9100265 | 7.4 |
| Breast Cancer A209073 | 20.4 |
| Breast Margin A209073 | 11.4 |
| Normal Liver | 11.1 |
| Liver Cancer 064003 | 2.9 |
| Liver Cancer 1025 | 4.9 |
| Liver Cancer 1026 | 5.8 |
| Liver Cancer 6004-T | 5.8 |
| Liver Tissue 6004-N | 6.1 |
| Liver Cancer 6005-T | 8.5 |
| Liver Tissue 6005-N | 1.3 |
| Normal Bladder | 15.3 |
| Bladder Cancer 1023 | 2.9 |
| Bladder Cancer A302173 | 8.5 |
| Bladder Cancer (OD04718-01) | 14.1 |
| Bladder Normal Adjacent (OD04718-03) | 18.9 |
| Normal Ovary | 6.7 |
| Ovarian Cancer 064008 | 32.3 |
| Ovarian Cancer (OD04768-07) | 6.1 |
| Ovary Margin (OD04768-08) | 4.6 |
| Normal Stomach | 5.6 |
| Gastric Cancer 9060358 | 3.6 |
| Stomach Margin 9060359 | 5.3 |
| Gastric Cancer 9060395 | 8.1 |
| Stomach Margin 9060394 | 4.5 |
| Gastric Cancer 9060397 | 11.2 |
| Stomach Margin 9060396 | 2.0 |
| Gastric Cancer 064005 | 19.2 |

Column A - Rel. Exp. (%) Ag1252, Run 162309309

TABLE EG
Panel 4D

| Tissue Name | A |
|---|---|
| Secondary Th1 act | 1.0 |
| Secondary Th2 act | 4.6 |
| Secondary Tr1 act | 1.6 |
| Secondary Th1 rest | 1.7 |
| Secondary Th2 rest | 4.1 |
| Secondary Tr1 rest | 2.3 |
| Primary Th1 act | 4.6 |
| Primary Th2 act | 4.5 |
| Primary Tr1 act | 9.5 |
| Primary Th1 rest | 12.9 |
| Primary Th2 rest | 8.5 |
| Primary Tr1 rest | 15.8 |
| CD45RA CD4 lymphocyte act | 12.6 |
| CD45RO CD4 lymphocyte act | 5.6 |
| CD8 lymphocyte act | 5.4 |
| Secondary CD8 lymphocyte rest | 3.2 |
| Secondary CD8 lymphocyte act | 1.9 |
| CD4 lymphocyte none | 4.9 |
| 2ry Th1/Th2/Tr1_anti-CD95 CH11 | 11.9 |
| LAK cells rest | 47.3 |
| LAK cells IL-2 | 1.9 |
| LAK cells IL-2 + IL-12 | 5.8 |
| LAK cells IL-2 + IFN gamma | 6.0 |
| LAK cells IL-2 + IL-18 | 5.5 |
| LAK cells PMA/ionomycin | 15.2 |
| NK Cells IL-2 rest | 1.1 |
| Two Way MLR 3 day | 20.9 |
| Two Way MLR 5 day | 8.7 |
| Two Way MLR 7 day | 1.1 |
| PBMC rest | 14.6 |
| PBMC PWM | 15.2 |
| PBMC PHA-L | 7.0 |
| Ramos (B cell) none | 4.0 |
| Ramos (B cell) ionomycin | 7.6 |
| B lymphocytes PWM | 8.2 |
| B lymphocytes CD40L and IL-4 | 8.5 |
| EOL-1 dbcAMP | 33.4 |
| EOL-1 dbcAMP PMA/ionomycin | 82.9 |
| Dendritic cells none | 28.1 |
| Dendritic cells LPS | 12.8 |
| Dendritic cells anti-CD40 | 35.1 |
| Monocytes rest | 35.8 |
| Monocytes LPS | 54.0 |
| Macrophages rest | 100.0 |
| Macrophages LPS | 23.3 |
| HUVEC none | 37.6 |
| HUVEC starved | 70.2 |
| HUVEC IL-1beta | 23.3 |
| HUVEC IFN gamma | 55.9 |
| HUVEC TNF alpha + IFN gamma | 8.6 |
| HUVEC TNF alpha + IL4 | 21.5 |
| HUVEC IL-11 | 31.9 |
| Lung Microvascular EC none | 58.6 |
| Lung Microvascular EC TNFalpha + IL-1beta | 32.1 |
| Microvascular Dermal EC none | 84.7 |
| Microsvasular Dermal EC TNFalpha + IL-1beta | 41.8 |
| Bronchial epithelium TNFalpha + IL1beta | 27.4 |
| Small airway epithelium none | 13.3 |
| Small airway epithelium TNFalpha + IL-1beta | 61.6 |
| Coronery artery SMC rest | 50.7 |
| Coronery artery SMC TNFalpha + IL-1beta | 20.3 |
| Astrocytes rest | 7.0 |
| Astrocytes TNFalpha + IL-1beta | 13.2 |
| KU-812 (Basophil) rest | 9.2 |
| KU-812 (Basophil) PMA/ionomycin | 5.3 |
| CCD1106 (Keratinocytes) none | 15.3 |
| CCD1106 (Keratinocytes) TNFalpha + IL-1beta | 53.2 |
| Liver cirrhosis | 8.9 |
| Lupus kidney | 10.9 |
| NCI-H292 none | 42.3 |
| NCI-H292 IL-4 | 34.2 |

Column A - Rel. Exp. (%) Ag1252, Run 141902582

TABLE EH
Panel CNS_

| Tissue Name | A |
|---|---|
| BA4 Control | 46.3 |
| BA4 Control2 | 77.9 |
| BA4 Alzheimer's2 | 4.7 |
| BA4 Parkinson's | 64.2 |
| BA4 Parkinson's2 | 80.1 |
| BA4 Huntington's | 37.6 |
| BA4 Huntington's2 | 12.2 |
| BA4 PSP | 22.5 |
| BA4 PSP2 | 39.8 |
| BA4 Depression | 28.9 |
| BA4 Depression2 | 4.2 |
| BA7 Control | 62.0 |
| BA7 Control2 | 95.3 |
| BA7 Alzheimer's2 | 5.1 |
| BA7 Parkinson's | 23.0 |
| BA7 Parkinson's2 | 55.1 |
| BA7 Huntington's | 56.3 |
| BA7 Huntington's2 | 44.1 |
| BA7 PSP | 32.5 |
| BA7 PSP2 | 44.4 |
| BA7 Depression | 8.1 |
| BA9 Control | 33.9 |
| BA9 Control2 | 77.9 |
| BA9 Alzheimer's | 0.0 |
| BA9 Alzheimer's2 | 26.8 |
| BA9 Parkinson's | 16.3 |
| BA9 Parkinson's2 | 37.1 |
| BA9 Huntington's | 100.0 |
| BA9 Huntington's2 | 35.6 |
| BA9 PSP | 13.7 |
| BA9 PSP2 | 9.4 |
| BA9 Depression | 7.1 |
| BA9 Depression2 | 18.3 |
| BA17 Control | 39.2 |
| BA17 Control2 | 26.1 |
| BA17 Alzheimer's2 | 5.2 |
| BA17 Parkinson's | 65.1 |
| BA17 Parkinson's2 | 51.8 |
| BA17 Huntington's | 43.2 |
| BA17 Huntington's2 | 12.3 |
| BA17 Depression | 16.7 |
| BA17 Depression2 | 5.8 |
| BA17 PSP | 12.2 |
| BA17 PSP2 | 1.4 |
| Sub Nigra Control | 28.9 |
| Sub Nigra Control2 | 44.1 |
| Sub Nigra Alzheimer's2 | 25.0 |
| Sub Nigra Parkinson's2 | 34.9 |
| Sub Nigra Huntington's | 67.4 |
| Sub Nigra Huntington's2 | 32.8 |
| Sub Nigra PSP2 | 9.7 |
| Sub Nigra Depression | 0.0 |
| Sub Nigra Depression2 | 1.1 |
| Glob Palladus Control | 14.4 |
| Glob Palladus Control2 | 31.2 |
| Glob Palladus Alzheimer's | 12.2 |
| Glob Palladus Alzheimer's2 | 0.0 |
| Glob Palladus Parkinson's | 71.7 |
| Glob Palladus Parkinson's2 | 19.8 |
| Glob Palladus PSP | 8.4 |
| Glob Palladus PSP2 | 12.8 |
| Glob Palladus Depression | 0.9 |
| Temp Pole Control | 14.7 |
| Temp Pole Control2 | 46.3 |
| Temp Pole Alzheimer's | 17.4 |
| Temp Pole Alzheimer's2 | 0.0 |
| Temp Pole Parkinson's | 26.4 |
| Temp Pole Parkinson's2 | 36.3 |
| Temp Pole Huntington's | 82.9 |
| Temp Pole PSP | 5.9 |
| Temp Pole PSP2 | 5.2 |
| Temp Pole Depression2 | 5.7 |
| Cing Gyr Control | 63.7 |
| Cing Gyr Control2 | 32.1 |
| Cing Gyr Alzheimer's | 22.1 |
| Cing Gyr Alzheimer's2 | 1.2 |
| Cing Gyr Parkinson's | 15.7 |
| Cing Gyr Parkinson's2 | 54.0 |
| Cing Gyr Huntington's | 58.2 |
| Cing Gyr Huntington's2 | 25.0 |
| Cing Gyr PSP | 26.8 |
| Cing Gyr PSP2 | 17.0 |
| Cing Gyr Depression | 3.3 |
| Cing Gyr Depression2 | 1.4 |

Column A - Rel. Exp. (%) Ag1252, Run 171629874

AI_comprehensive panel_v1.0 Summary: Ag1252 Highest expression of this gene is seen in matched control psoriasis sample (CT=32.8). Low expression of this gene is also seen in osteoarthitis/rheumatoid arthritis bone, cartilage, synovium and synovial fluid samples, from normal lung, emphysema, atopic asthma, asthma, Crohn's disease (normal matched control and diseased), and psoriasis (normal matched control and diseased). Therefore, therapeutic modulation of this gene product may ameliorate symptoms/conditions associated with autoimmune and inflammatory disorders including psoriasis, asthma, inflammatory bowel disease, rheumatoid arthritis and osteoarthritis.

CNS_neurodegeneration_v1.0 Summary: Data obtained from RTQPCR assays performed for this panel was analyized using multivariate analysis (ANOVA). The multivariate results obtained using Ag1252 in two experiments with the same probe-primer sets are in good agreement. This gene is found to be down-regulated in the temporal cortex of Alzheimer's disease patients. Therefore, up-regulation of this gene or its protein product, or treatment with specific agonists for this receptor may be of use in reversing the dementia/memory loss associated with this disease and neuronal death.

General_screening_panel_v1.4 Summary: Ag1252 This gene shows ubiquitous expression in this panel with highest expression in brain cerebellum (CT=26.8).This gene is expressed at high to moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Moderate levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Panel 1.2 Summary: Ag1252 The expression of this gene shows a significant level of expression across the majority of samples in the panel and correlates with expression seen in panel 1.4. Of interest is the observation that lung cancer cell lines appear to show considerably higher expression compared to other samples, suggesting that this gene may play a role in lung cancer. Please see panel 1.4 for further discussion in the role of this gene.

Panel 2D Summary: Ag1213 This gene shows widespread expression in this panel. Highest expression of this gene is seen in a breast cancer (CT=29.5). There is a slight up-regulation of this gene in lung, kidney, breast and gastric cancer when compared to corresponding normal adjacent controls. Thus, therapies targeted towards this gene or its protein product may be beneficial to the treatment of lung, kidney, breast and gastric cancers.

Panel 4D Summary: Ag1213 Highest expression of this gene is seen in resting macrophage (CT=30.6). This gene shows wide spread expression with higher expression in resting LAK cells, eosinophils, dendritic cells, monocytes, macrophages, endothelial cells, bronchial and small airway epithelial cells, keratinocytes, mucoepidermoid NCI-H292 cells, lung and dermal fibroblasts and normal tissues represented by colon, lung, and thymus. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel CNS_{—}1 Summary: Ag1213 This gene encodes for a homolog of serotinin receptor 7. Serotonin receptors have been implicated in neuropsychiatric disorders including schizophrenia, bipolar disorder, depression, and Alzheimer's disease. This gene is downregulated in the cingulate gyrus, parietal cortex, and substantia nigra in depression as measured via RTQ-PCR analysis in postmortem brain tissue. Because many antidepressants are serotonin reuptake inhibitors (e.g., fluvoxamine, hypericum perforatum, clomipramine, milnacipran, etc) the downregulation of a serotonin receptor in the brains of patients suffering from chronic depression suggests that this molecule may be a primary role in the etiology of this disease and be an excellent small molecule target for the treatment of psychiatric disease.

F. CG55912-01: CACNG4-Ion Channel

Expression of gene CG55912-01 was assessed using the primer-probe set Ag2841, described in Table FA. Results of the RTQ-PCR runs are shown in Tables FB, FC, FD, FE, FF and FG.

TABLE FA

Probe Name Ag2841

| | | | Start | SEQ ID |
|---|---|---|---|---|
| Primers | Sequences | Length | Position | No |
| Forward | 5′-gtctgcgtgaagatcaatcatt-3′ | 22 | 283 | 113 |
| Probe | TET-5′-aggacacggactacgaccacgacag-3′-TAMRA | 25 | 311 | 114 |
| Reverse | 5′-cggaccgtacggagtagatact-3′ | 22 | 341 | 115 |

TABLE FB
CNS_neurodegeneration_v1.0

| Tissue Name | A |
|---|---|
| AD 1 Hippo | 15.8 |
| AD 2 Hippo | 34.6 |
| AD 3 Hippo | 15.9 |
| AD 4 Hippo | 17.0 |
| AD 5 hippo | 85.3 |
| AD 6 Hippo | 51.1 |
| Control 2 Hippo | 21.0 |
| Control 4 Hippo | 0.0 |
| Control (Path) 3 Hippo | 0.0 |
| AD 1 Temporal Ctx | 6.3 |
| AD 2 Temporal Ctx | 16.3 |
| AD 3 Temporal Ctx | 5.0 |
| AD 4 Temporal Ctx | 7.9 |
| AD 5 Inf Temporal Ctx | 64.6 |
| AD 5 SupTemporal Ctx | 38.4 |
| AD 6 Inf Temporal Ctx | 46.3 |
| AD 6 Sup Temporal Ctx | 88.3 |
| Control 1 Temporal Ctx | 10.4 |
| Control 2 Temporal Ctx | 42.9 |
| Control 3 Temporal Ctx | 20.3 |
| Control 4 Temporal Ctx | 8.4 |
| Control (Path) 1 Temporal Ctx | 58.2 |
| Control (Path) 2 Temporal Ctx | 49.7 |
| Control (Path) 3 Temporal Ctx | 0.0 |
| Control (Path) 4 Temporal Ctx | 25.9 |
| AD 1 Occipital Ctx | 6.8 |
| AD 2 Occipital Ctx (Missing) | 0.0 |
| AD 3 Occipital Ctx | 24.0 |
| AD 4 Occipital Ctx | 21.8 |
| AD 5 Occipital Ctx | 0.0 |
| AD 6 Occipital Ctx | 51.8 |
| Control 1 Occipital Ctx | 6.0 |
| Control 2 Occipital Ctx | 100.0 |
| Control 3 Occipital Ctx | 39.2 |
| Control 4 Occipital Ctx | 5.1 |
| Control (Path) 1 Occipital Ctx | 46.7 |
| Control (Path) 2 Occipital Ctx | 23.0 |
| Control (Path) 3 Occipital Ctx | 6.3 |
| Control (Path) 4 Occipital Ctx | 25.3 |
| Control 1 Parietal Ctx | 0.0 |
| Control 2 Parietal Ctx | 51.4 |
| Control 3 Parietal Ctx | 18.9 |
| Control (Path) 1 Parietal Ctx | 86.5 |
| Control (Path) 2 Parietal Ctx | 31.0 |
| Control (Path) 3 Parietal Ctx | 7.5 |
| Control (Path) 4 Parietal Ctx | 63.7 |

Column A - Rel. Exp. (%) Ag2841, Run 209779166

TABLE FC
Panel 1.3D

| Tissue Name | A | B |
|---|---|---|
| Liver adenocarcinoma | 3.1 | 7.2 |
| Pancreas | 0.0 | 0.0 |
| Pancreatic ca. CAPAN 2 | 5.7 | 5.9 |
| Adrenal gland | 0.0 | 0.0 |
| Thyroid | 0.0 | 0.0 |
| Salivary gland | 0.0 | 0.0 |
| Pituitary gland | 0.0 | 0.0 |
| Brain (fetal) | 18.3 | 27.0 |
| Brain (whole) | 27.0 | 19.5 |
| Brain (amygdala) | 77.4 | 54.3 |
| Brain (cerebellum) | 0.0 | 0.0 |
| Brain (hippocampus) | 100.0 | 100.0 |
| Brain (substantia nigra) | 0.0 | 3.5 |
| Brain (thalamus) | 7.9 | 0.0 |
| Cerebral Cortex | 90.8 | 8.4 |
| Spinal cord | 3.9 | 0.0 |
| glio/astro U87-MG | 2.7 | 6.5 |
| glio/astro U-118-MG | 0.0 | 0.0 |
| astrocytoma SW1783 | 0.0 | 6.6 |
| neuro*; met SK-N-AS | 8.4 | 0.0 |
| astrocytoma SF-539 | 0.0 | 0.0 |
| astrocytoma SNB-75 | 0.0 | 0.0 |
| glioma SNB-19 | 27.2 | 6.2 |
| glioma U251 | 0.0 | 0.0 |
| glioma SF-295 | 0.0 | 3.4 |
| Heart (fetal) | 0.0 | 0.0 |
| Heart | 0.0 | 0.0 |
| Skeletal muscle (fetal) | 0.0 | 0.0 |
| Skeletal muscle | 0.0 | 0.0 |
| Bone marrow | 3.8 | 0.0 |
| Thymus | 0.0 | 0.0 |
| Spleen | 0.0 | 0.0 |
| Lymph node | 0.0 | 0.0 |
| Colorectal | 0.0 | 0.0 |
| Stomach | 0.0 | 0.0 |
| Small intestine | 0.0 | 0.0 |
| Colon ca. SW480 | 0.0 | 2.6 |
| Colon ca.* SW620 (SW480 met) | 0.0 | 0.0 |
| Colon ca. HT29 | 0.0 | 0.0 |
| Colon ca. HCT-116 | 0.0 | 0.0 |
| Colon ca. CaCo-2 | 0.0 | 0.0 |
| Colon ca. tissue (ODO3866) | 0.0 | 0.0 |
| Colon ca. HCC-2998 | 8.4 | 0.0 |
| Gastric ca.* (liver met) NCI-N87 | 18.0 | 4.2 |
| Bladder | 0.0 | 0.0 |
| Trachea | 0.0 | 0.0 |
| Kidney | 0.0 | 0.0 |
| Kidney (fetal) | 0.0 | 0.0 |
| Renal ca. 786-0 | 0.0 | 0.0 |
| Renal ca. A498 | 0.0 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.0 | 0.0 |
| Liver (fetal) | 0.0 | 0.0 |
| Liver ca. (hepatoblast) HepG2 | 0.0 | 0.0 |
| Lung | 3.0 | 9.2 |
| Lung (fetal) | 0.0 | 0.0 |
| Lung ca. (small cell) LX-1 | 0.0 | 0.0 |
| Lung ca. (small cell) NCI-H69 | 0.0 | 0.0 |
| Lung ca. (s. cell var.) SHP-77 | 0.0 | 3.9 |
| Lung ca. (large cell) NCI-H460 | 0.0 | 0.0 |
| Lung ca. (non-sm. cell) A549 | 15.0 | 0.0 |
| Lung ca. (non-s. cell) NCI-H23 | 7.2 | 12.5 |
| Lung ca. (non-s. cell) HOP-62 | 0.0 | 0.0 |
| Lung ca. (non-s. cl) NCI-H522 | 12.9 | 3.2 |
| Lung ca. (squam.) SW 900 | 0.0 | 0.0 |
| Lung ca. (squam.) NCI-H596 | 0.0 | 0.0 |
| Mammary gland | 0.0 | 0.0 |
| Breast ca.* (pl. ef) MCF-7 | 0.0 | 0.0 |
| Breast ca.* (pl. ef) MDA-MB-231 | 0.0 | 0.0 |
| Breast ca.* (pl. ef) T47D | 0.0 | 0.0 |
| Breast ca. BT-549 | 0.0 | 0.0 |
| Breast ca. MDA-N | 0.0 | 0.0 |
| Ovary | 0.0 | 0.0 |
| Ovarian ca. OVCAR-3 | 0.0 | 0.0 |
| Ovarian ca. OVCAR-4 | 0.0 | 22.1 |
| Ovarian ca. OVCAR-5 | 7.0 | 0.0 |
| Ovarian ca. OVCAR-8 | 0.0 | 0.0 |
| Ovarian ca. IGROV-1 | 0.0 | 0.0 |
| Ovarian ca.* (ascites) SK-OV-3 | 0.0 | 0.0 |
| Uterus | 0.0 | 2.6 |
| Placenta | 0.0 | 0.0 |
| Prostate | 0.0 | 0.0 |
| Prostate ca.* (bone met) PC-3 | 0.0 | 2.0 |
| Testis | 21.0 | 0.0 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 11.0 |
| Melanoma* (met) SK-MEL-5 | 0.0 | 0.0 |
| Adipose | 0.0 | 0.0 |

Column A - Rel. Exp. (%) Ag2841, Run 161922470
Column B - Rel. Exp. (%) Ag2841, Run 165721032

TABLE FD
Panel 2D

| Tissue Name | A |
|---|---|
| Normal Colon | 0.0 |
| CC Well to Mod Diff (ODO3866) | 0.0 |
| CC Margin (ODO3866) | 20.2 |
| CC Gr.2 rectosigmoid (ODO3868) | 0.0 |
| CC Margin (ODO3868) | 0.0 |
| CC Mod Diff (ODO3920) | 100.0 |
| CC Margin (ODO3920) | 0.0 |
| CC Gr.2 ascend colon (ODO3921) | 41.8 |
| CC Margin (ODO3921) | 0.0 |
| CC from Partial Hepatectomy (ODO4309) Mets | 0.0 |
| Liver Margin (ODO4309) | 0.0 |
| Colon mets to lung (OD04451-01) | 0.0 |
| Lung Margin (OD04451-02) | 0.0 |
| Normal Prostate 6546-1 | 0.0 |
| Prostate Cancer (OD04410) | 0.0 |
| Prostate Margin (OD04410) | 0.0 |
| Prostate Cancer (OD04720-01) | 0.0 |
| Prostate Margin (OD04720-02) | 0.0 |
| Normal Lung 061010 | 55.9 |
| Lung Met to Muscle (ODO4286) | 0.0 |
| Muscle Margin (ODO4286) | 0.0 |
| Lung Malignant Cancer (OD03126) | 0.0 |
| Lung Margin (OD03126) | 0.0 |
| Lung Cancer (OD04404) | 0.0 |
| Lung Margin (OD04404) | 0.0 |
| Lung Cancer (OD04565) | 0.0 |
| Lung Margin (OD04565) | 0.0 |
| Lung Cancer (OD04237-01) | 0.0 |
| Lung Margin (OD04237-02) | 0.0 |
| Ocular Mel Met to Liver (ODO4310) | 0.0 |
| Liver Margin (ODO4310) | 0.0 |
| Melanoma Mets to Lung (OD04321) | 0.0 |
| Lung Margin (OD04321) | 0.0 |
| Normal Kidney | 0.0 |
| Kidney Ca, Nuclear grade 2 (OD04338) | 0.0 |
| Kidney Margin (OD04338) | 0.0 |
| Kidney Ca Nuclear grade 1/2 (OD04339) | 0.0 |
| Kidney Margin (OD04339) | 0.0 |
| Kidney Ca, Clear cell type (OD04340) | 0.0 |
| Kidney Margin (OD04340) | 0.0 |
| Kidney Ca, Nuclear grade 3 (OD04348) | 0.0 |
| Kidney Margin (OD04348) | 0.0 |
| Kidney Cancer (OD04622-01) | 0.0 |
| Kidney Margin (OD04622-03) | 0.0 |
| Kidney Cancer (OD04450-01) | 0.0 |
| Kidney Margin (OD04450-03) | 0.0 |
| Kidney Cancer 8120607 | 0.0 |
| Kidney Margin 8120608 | 0.0 |
| Kidney Cancer 8120613 | 0.0 |
| Kidney Margin 8120614 | 0.0 |
| Kidney Cancer 9010320 | 0.0 |
| Kidney Margin 9010321 | 0.0 |
| Normal Uterus | 0.0 |
| Uterus Cancer 064011 | 0.0 |
| Normal Thyroid | 0.0 |
| Thyroid Cancer 064010 | 19.1 |
| Thyroid Cancer A302152 | 0.0 |
| Thyroid Margin A302153 | 0.0 |
| Normal Breast | 10.5 |
| Breast Cancer (OD04566) | 0.0 |
| Breast Cancer (OD04590-01) | 0.0 |
| Breast Cancer Mets (OD04590-03) | 0.0 |
| Breast Cancer Metastasis (OD04655-05) | 0.0 |
| Breast Cancer 064006 | 0.0 |
| Breast Cancer 1024 | 45.4 |
| Breast Cancer 9100266 | 0.0 |
| Breast Margin 9100265 | 0.0 |
| Breast Cancer A209073 | 0.0 |
| Breast Margin A209073 | 49.3 |
| Normal Liver | 0.0 |
| Liver Cancer 064003 | 23.3 |
| Liver Cancer 1025 | 0.0 |
| Liver Cancer 1026 | 0.0 |
| Liver Cancer 6004-T | 0.0 |
| Liver Tissue 6004-N | 0.0 |
| Liver Cancer 6005-T | 0.0 |
| Liver Tissue 6005-N | 0.0 |
| Normal Bladder | 21.6 |
| Bladder Cancer 1023 | 0.0 |
| Bladder Cancer A302173 | 14.3 |
| Bladder Cancer (OD04718-01) | 0.0 |
| Bladder Normal Adjacent (OD04718-03) | 0.0 |
| Normal Ovary | 0.0 |
| Ovarian Cancer 064008 | 16.3 |
| Ovarian Cancer (OD04768-07) | 57.8 |
| Ovary Margin (OD04768-08) | 0.0 |
| Normal Stomach | 0.0 |
| Gastric Cancer 9060358 | 0.0 |
| Stomach Margin 9060359 | 0.0 |
| Gastric Cancer 9060395 | 0.0 |
| Stomach Margin 9060394 | 0.0 |
| Gastric Cancer 9060397 | 0.0 |
| Stomach Margin 9060396 | 0.0 |
| Gastric Cancer 064005 | 0.0 |

Column A - Rel. Exp. (%) Ag2841, Run 161930423

TABLE FE
Panel 3D

| Tissue Name | A |
|---|---|
| Daoy- Medulloblastoma | 11.6 |
| TE671- Medulloblastoma | 7.0 |
| D283 Med- Medulloblastoma | 0.0 |
| PFSK-1- Primitive Neuroectodermal | 0.0 |
| XF-498- CNS | 0.0 |
| SNB-78- Glioma | 0.0 |
| SF-268- Glioblastoma | 12.8 |
| T98G- Glioblastoma | 0.0 |
| SK-N-SH- Neuroblastoma (metastasis) | 39.0 |
| SF-295- Glioblastoma | 0.0 |
| Cerebellum | 0.0 |
| Cerebellum | 25.7 |
| NCI-H292- Mucoepidermoid lung carcinoma | 0.0 |
| DMS-114- Small cell lung cancer | 10.7 |
| DMS-79- Small cell lung cancer | 100.0 |
| NCI-H146- Small cell lung cancer | 0.0 |
| NCI-H526- Small cell lung cancer | 16.2 |
| NCI-N417- Small cell lung cancer | 0.0 |
| NCI-H82- Small cell lung cancer | 0.0 |
| NCI-H157- Squamous cell lung cancer (metastasis) | 7.5 |
| NCI-H1155- Large cell lung cancer | 16.8 |
| NCI-H1299- Large cell lung cancer | 39.8 |
| NCI-H727- Lung carcinoid | 10.3 |
| NCI-UMC-11- Lung carcinoid | 0.0 |
| LX-1- Small cell lung cancer | 0.0 |
| Colo-205- Colon cancer | 0.0 |
| KM12- Colon cancer | 25.7 |
| KM20L2- Colon cancer | 21.6 |
| NCI-H716- Colon cancer | 0.0 |
| SW-48- Colon adenocarcinoma | 0.0 |
| SW1116- Colon adenocarcinoma | 0.0 |
| LS 174T- Colon adenocarcinoma | 0.0 |
| SW-948- Colon adenocarcinoma | 0.0 |
| SW-480- Colon adenocarcinoma | 0.0 |
| NCI-SNU-5- Gastric carcinoma | 21.6 |
| KATO III- Gastric carcinoma | 4.2 |
| NCI-SNU-16- Gastric carcinoma | 0.0 |
| NCI-SNU-1- Gastric carcinoma | 0.0 |
| RF-1- Gastric adenocarcinoma | 0.0 |
| RF-48- Gastric adenocarcinoma | 0.0 |
| MKN-45- Gastric carcinoma | 39.5 |
| NCI-N87- Gastric carcinoma | 0.0 |
| OVCAR-5- Ovarian carcinoma | 0.0 |
| RL95-2- Uterine carcinoma | 11.4 |
| HelaS3- Cervical adenocarcinoma | 0.0 |
| Ca Ski- Cervical epidermoid carcinoma (metastasis) | 0.0 |
| ES-2- Ovarian clear cell carcinoma | 3.6 |
| Ramos- Stimulated with PMA/ionomycin 6 h | 0.0 |
| Ramos- Stimulated with PMA/ionomycin 14 h | 0.0 |
| MEG-01- Chronic myelogenous leukemia (megokaryoblast) | 3.6 |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.0 |
| HUT 78- T-cell lymphoma | 0.0 |
| U937- Histiocytic lymphoma | 0.0 |
| KU-812- Myelogenous leukemia | 0.0 |
| 769-P- Clear cell renal carcinoma | 0.0 |
| Caki-2- Clear cell renal carcinoma | 3.8 |
| SW 839- Clear cell renal carcinoma | 0.0 |
| Rhabdoid kidney tumor | 36.6 |
| Hs766T- Pancreatic carcinoma (LN metastasis) | 14.3 |
| CAPAN-1- Pancreatic adenocarcinoma (liver metastasis) | 6.7 |
| SU86.86- Pancreatic carcinoma (liver metastasis) | 0.0 |
| BxPC-3- Pancreatic adenocarcinoma | 0.0 |
| HPAC- Pancreatic adenocarcinoma | 0.0 |
| MIA PaCa-2- Pancreatic carcinoma | 25.7 |
| CFPAC-1- Pancreatic ductal adenocarcinoma | 35.4 |
| PANC-1- Pancreatic epithelioid ductal carcinoma | 40.9 |
| T24- Bladder carcinma (transitional cell) | 0.0 |
| 5637- Bladder carcinoma | 0.0 |
| HT-1197- Bladder carcinoma | 0.0 |
| UM-UC-3- Bladder carcinma (transitional cell) | 12.2 |
| A204- Rhabdomyosarcoma | 0.0 |
| HT-1080- Fibrosarcoma | 4.6 |
| MG-63- Osteosarcoma | 0.0 |
| SK-LMS-1- Leiomyosarcoma (vulva) | 31.4 |
| SJRH30- Rhabdomyosarcoma (met to bone marrow) | 0.0 |
| A431- Epidermoid carcinoma | 0.0 |
| WM266-4- Melanoma | 0.0 |
| DU 145- Prostate carcinoma (brain metastasis) | 6.7 |
| MDA-MB-468- Breast adenocarcinoma | 0.0 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

Column A - Rel. Exp. (%) Ag2841, Run 164538045

TABLE FF
Panel 4D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1 anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 5.9 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 5.8 |
| HUVEC starved | 6.9 |
| HUVEC IL-1beta | 2.7 |
| HUVEC IFN gamma | 8.8 |
| HUVEC TNF alpha + IFN gamma | 15.9 |
| HUVEC TNF alpha + IL4 | 2.2 |
| HUVEC IL-11 | 13.6 |
| Lung Microvascular EC none | 21.5 |
| Lung Microvascular EC TNFalpha + IL-1beta | 33.7 |
| Microvascular Dermal EC none | 12.1 |
| Microsvasular Dermal EC TNFalpha + IL-1beta | 0.0 |
| Bronchial epithelium TNFalpha + IL1beta | 0.0 |
| Small airway epithelium none | 2.4 |
| Small airway epithelium TNFalpha + IL-1beta | 0.0 |
| Coronery artery SMC rest | 10.8 |
| Coronery artery SMC TNFalpha + IL-1beta | 8.3 |
| Astrocytes rest | 0.0 |
| Astrocytes TNFalpha + IL-1beta | 9.8 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 15.1 |
| Lupus kidney | 0.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 19.6 |
| HPAEC TNF alpha + IL-1 beta | 7.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 3.6 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| IBD Colitis 2 | 0.0 |
| IBD Crohn's | 0.0 |
| Colon | 100.0 |
| Lung | 40.1 |
| Thymus | 0.0 |
| Kidney | 0.0 |

Column A - Rel. Exp. (%) Ag2841, Run 159616564

TABLE FG
Panel CNS_1

| Tissue Name | A |
|---|---|
| BA4 Control | 0.0 |
| BA4 Control2 | 9.2 |
| BA4 Alzheimer's2 | 0.0 |
| BA4 Parkinson's | 47.6 |
| BA4 Parkinson's2 | 50.3 |
| BA4 Huntington's | 13.2 |
| BA4 Huntington's2 | 11.7 |
| BA4 PSP | 38.2 |
| BA4 PSP2 | 14.4 |
| BA4 Depression | 0.0 |
| BA4 Depression2 | 0.0 |
| BA7 Control | 10.1 |
| BA7 Control2 | 0.0 |
| BA7 Alzheimer's2 | 0.0 |
| BA7 Parkinson's | 51.4 |
| BA7 Parkinson's2 | 17.4 |
| BA7 Huntington's | 46.3 |
| BA7 Huntington's2 | 36.3 |
| BA7 PSP | 46.3 |
| BA7 PSP2 | 37.9 |
| BA7 Depression | 29.9 |
| BA9 Control | 0.0 |
| BA9 Control2 | 38.2 |
| BA9 Alzheimer's | 8.2 |
| BA9 Alzheimer's2 | 41.2 |
| BA9 Parkinson's | 100.0 |
| BA9 Parkinson's2 | 54.3 |
| BA9 Huntington's | 9.3 |
| BA9 Huntington's2 | 57.8 |
| BA9 PSP | 0.0 |
| BA9 PSP2 | 0.0 |
| BA9 Depression | 0.0 |
| BA9 Depression2 | 14.1 |
| BA17 Control | 51.4 |
| BA17 Control2 | 25.9 |
| BA17 Alzheimer's2 | 0.0 |
| BA17 Parkinson's | 16.3 |
| BA17 Parkinson's2 | 47.3 |
| BA17 Huntington's | 89.5 |
| BA17 Huntington's2 | 13.0 |
| BA17 Depression | 0.0 |
| BA17 Depression2 | 66.4 |
| BA17 PSP | 9.2 |
| BA17 PSP2 | 0.0 |
| Sub Nigra Control | 8.7 |
| Sub Nigra Control2 | 35.1 |
| Sub Nigra Alzheimer's2 | 0.0 |
| Sub Nigra Parkinson's2 | 0.0 |
| Sub Nigra Huntington's | 10.0 |
| Sub Nigra Huntington's2 | 25.9 |
| Sub Nigra PSP2 | 0.0 |
| Sub Nigra Depression | 0.0 |
| Sub Nigra Depression2 | 0.0 |
| Glob Palladus Control | 0.0 |
| Glob Palladus Control2 | 0.0 |
| Glob Palladus Alzheimer's | 0.0 |
| Glob Palladus Alzheimer's2 | 0.0 |
| Glob Palladus Parkinson's | 18.2 |
| Glob Palladus Parkinson's2 | 0.0 |
| Glob Palladus PSP | 0.0 |
| Glob Palladus PSP2 | 0.0 |
| Glob Palladus Depression | 0.0 |
| Temp Pole Control | 20.3 |
| Temp Pole Control2 | 28.5 |
| Temp Pole Alzheimer's | 0.0 |
| Temp Pole Alzheimer's2 | 0.0 |
| Temp Pole Parkinson's | 17.2 |
| Temp Pole Parkinson's2 | 0.0 |
| Temp Pole Huntington's | 51.4 |
| Temp Pole PSP | 0.0 |
| Temp Pole PSP2 | 0.0 |
| Temp Pole Depression2 | 21.5 |
| Cing Gyr Control | 51.4 |
| Cing Gyr Control2 | 0.0 |
| Cing Gyr Alzheimer's | 0.0 |
| Cing Gyr Alzheimer's2 | 0.0 |
| Cing Gyr Parkinson's | 38.7 |
| Cing Gyr Parkinson's2 | 13.3 |
| Cing Gyr Huntington's | 27.2 |
| Cing Gyr Huntington's2 | 30.4 |
| Cing Gyr PSP | 0.0 |
| Cing Gyr PSP2 | 0.0 |
| Cing Gyr Depression | 0.0 |
| Cing Gyr Depression2 | 0.0 |

Column A - Rel. Exp. (%) Ag2841, Run 171669732

CNS_neurodegeneration_v1.0 Summary: Data obtained from RTQPCR assays performed for this panel was analyzed using multivariate analysis (ANOVA). The multivariate results obtained using Ag2841 indicates expression of this gene is low (CTs>35) across all of the samples on this panel. Although levels are low for this gene, there is a significant difference in expression levels between non-demented controls and patients suffering from Alzheimer's disease, such that the levels of mRNA appear to be downregulated 2-fold in the postmortem AD brain (p=0.0018 when analyzed by ANCOVA; estimate of RNA loaded per well used as a covariate). This gene may therefore represent a drug target for the treatment of Alzheimer's disease or other dementias.

Panel 1.3D Summary: Ag2841 Two experiments with same primer and probe set are in excellent agreement, with highest expression of this gene in brain hippocampus region (CT=34). Expression of this gene is exclusive to the brain region. In addition, this gene may play a role in central nervous system disorders such as Alzheimer's disease, Parkinson's disease, epilepsy, multiple sclerosis, schizophrenia and depression.

This gene codes for a homolog of neuronal voltage-gated calcium channel. In Caenorhabditis elegans voltage-gated calcium channels have been shown to direct neuronal migration. In C. elegans mutants carrying loss-of-function alleles of the calcium channel gene unc-2, the touch receptor neuron AVM and the intemeuron SDQR often migrated inappropriately, leading to misplacement of their cell bodies (Tam T, Mathews E, Snutch T P, Schafer W R. (2000) Voltage-gated calcium channels direct neuronal migration in Caenorhabditis elegans. Dev Biol 226(1):104-17, PMID: 10993677). Therefore, in analogy with C. elegan unc-2, neuronal voltage-gated calcium channel encoded by this gene may also play a role in directing neuronal migration. In addition, calcium channels have been implicated in number of neurological diseases such as familial hemiplegic migraine, episodic ataxia type 2, spinocerebellar ataxia 6, and Lambert-Eaton myasthenic syndrome and other diseases (Greenberg D A. (1997) Calcium channels in neurological disease. Ann Neurol 42(3):275-82, PMID: 9307247). Therefore, therapeutic modulation of the activity of this gene may prove useful in the treatment of the different neurological diseases.

Panel 4D Summary: Ag2841 Low but significant expression of this gene is detected exclusively in colon (CT=34). Therefore, expression of this gene may be used to distinguish colon from the other tissues on this panel. Furthermore, expression of this gene is decreased in colon samples from patients with IBD colitis and Crohn's disease relative to normal colon. Therefore, therapeutic modulation of the activity of the calcium channel encoded by this gene may be useful in the treatment of inflammatory bowel disease.

G. CG56001-01: 3-Hydroxybutyrate Dehydrogenase

Expression of gene CG56001-01 was assessed using the primer-probe set Ag2868, described in Table GA. Results of the RTQ-PCR runs are shown in Tables GB, GC, GD, GE and GF.

TABLE GA
Probe Name Ag2868

| | | | Start | SEQ ID |
|---|---|---|---|---|
| Primers | Sequences | Length | Position | No |
| Forward | 5′-ctactactggtggctgcgaat-3′ | 21 | 1025 | 116 |
| Probe | TET-5′-cagatcatgacccacttgcctggag-3′-TAMRA | 25 | 1047 | 117 |
| Reverse | 5′-actcttcagcggatgtagatca-3′ | 22 | 1084 | 118 |

TABLE GB
CNS_neurodegeneration_v1.0

| Tissue Name | A | B |
|---|---|---|
| AD 1 Hippo | 10.7 | 11.1 |
| AD 2 Hippo | 32.1 | 39.2 |
| AD 3 Hippo | 8.7 | 4.0 |
| AD 4 Hippo | 12.3 | 9.6 |
| AD 5 Hippo | 99.3 | 74.2 |
| AD 6 Hippo | 34.4 | 35.8 |
| Control 2 Hippo | 27.9 | 29.3 |
| Control 4 Hippo | 15.4 | 13.1 |
| Control (Path) 3 Hippo | 8.7 | 12.9 |
| AD 1 Temporal Ctx | 12.4 | 9.7 |
| AD 2 Temporal Ctx | 34.9 | 40.6 |
| AD 3 Temporal Ctx | 6.3 | 5.0 |
| AD 4 Temporal Ctx | 13.4 | 28.9 |
| AD 5 Inf Temporal Ctx | 100.0 | 100.0 |
| AD 5 Sup Temporal Ctx | 57.0 | 46.3 |
| AD 6 Inf Temporal Ctx | 32.3 | 33.4 |
| AD 6 Sup Temporal Ctx | 45.4 | 39.0 |
| Control 1 Temporal Ctx | 10.0 | 12.6 |
| Control 2 Temporal Ctx | 53.6 | 43.2 |
| Control 3 Temporal Ctx | 28.1 | 20.7 |
| Control 3 Temporal Ctx | 16.8 | 15.6 |
| Control (Path) 1 Temporal Ctx | 73.2 | 63.3 |
| Control (Path) 2 Temporal Ctx | 57.8 | 43.2 |
| Control (Path) 3 Temporal Ctx | 8.2 | 9.7 |
| Control (Path) 4 Temporal Ctx | 50.7 | 56.3 |
| AD 1 Occipital Ctx | 16.0 | 20.6 |
| AD 2 Occipital Ctx (Missing) | 0.0 | 0.0 |
| AD 3 Occipital Ctx | 6.3 | 8.4 |
| AD 4 Occipital Ctx | 24.8 | 22.7 |
| AD 5 Occipital Ctx | 48.3 | 19.3 |
| AD 6 Occipital Ctx | 18.2 | 43.5 |
| Control 1 Occipital Ctx | 6.6 | 5.5 |
| Control 2 Occipital Ctx | 67.4 | 60.7 |
| Control 3 Occipital Ctx | 26.2 | 28.9 |
| Control 4 Occipital Ctx | 7.6 | 5.5 |
| Control (Path) 1 Occipital Ctx | 92.0 | 68.8 |
| Control (Path) 2 Occipital Ctx | 18.9 | 15.6 |
| Control (Path) 3 Occipital Ctx | 3.8 | 2.7 |
| Control (Path) 4 Occipital Ctx | 35.1 | 30.6 |
| Control 1 Parietal Ctx | 15.5 | 10.1 |
| Control 2 Parietal Ctx | 47.0 | 36.3 |
| Control 3 Parietal Ctx | 17.3 | 29.5 |
| Control (Path) 1 Parietal Ctx | 94.6 | 77.9 |
| Control (Path) 2 Parietal Ctx | 35.4 | 41.2 |
| Control (Path) 3 Parietal Ctx | 4.3 | 6.2 |
| Control (Path) 4 Parietal Ctx | 68.8 | 59.5 |

Column A - Rel. Exp. (%) Ag2868, Run 206485413
Column B - Rel. Exp. (%) Ag2868, Run 224079571

TABLE GC
Panel 1.3D

| Tissue Name | A |
|---|---|
| Liver adenocarcinoma | 0.5 |
| Pancreas | 1.1 |
| Pancreatic ca. CAPAN 2 | 1.6 |
| Adrenal gland | 1.0 |
| Thyroid | 9.9 |
| Salivary gland | 7.0 |
| Pituitary gland | 3.0 |
| Brain (fetal) | 3.3 |
| Brain (whole) | 23.5 |
| Brain (amygdala) | 13.7 |
| Brain (cerebellum) | 28.3 |
| Brain (hippocampus) | 31.6 |
| Brain (substantia nigra) | 8.5 |
| Brain (thalamus) | 16.8 |
| Cerebral Cortex | 100.0 |
| Spinal cord | 10.2 |
| glio/astro U87-MG | 1.3 |
| glio/astro U-118-MG | 0.4 |
| astrocytoma SW1783 | 4.7 |
| neuro*; met SK-N-AS | 1.0 |
| astrocytoma SF-539 | 6.5 |
| astrocytoma SNB-75 | 1.4 |
| glioma SNB-19 | 11.5 |
| glioma U251 | 5.1 |
| glioma SF-295 | 0.5 |
| Heart (fetal) | 45.1 |
| Heart | 21.9 |
| Skeletal muscle (fetal) | 27.9 |
| Skeletal muscle | 20.2 |
| Bone marrow | 3.5 |
| Thymus | 30.1 |
| Spleen | 3.0 |
| Lymph node | 2.4 |
| Colorectal | 52.5 |
| Stomach | 6.7 |
| Small intestine | 17.1 |
| Colon ca. SW480 | 9.7 |
| Colon ca.* SW620(SW480 met) | 3.8 |
| Colon ca. HT29 | 13.1 |
| Colon ca. HCT-116 | 3.5 |
| Colon ca. CaCo-2 | 14.1 |
| Colon ca. tissue(ODO3866) | 17.8 |
| Colon ca. HCC-2998 | 18.0 |
| Gastric ca.* (liver met) NCI-N87 | 7.9 |
| Bladder | 5.6 |
| Trachea | 28.5 |
| Kidney | 29.9 |
| Kidney (fetal) | 9.1 |
| Renal ca. 786-0 | 0.0 |
| Renal ca. A498 | 1.1 |
| Renal ca. RXF 393 | 2.8 |
| Renal ca. ACHN | 1.5 |
| Renal ca. UO-31 | 2.7 |
| Renal ca. TK-10 | 3.4 |
| Liver | 36.9 |
| Liver (fetal) | 24.7 |
| Liver ca. (hepatoblast) HepG2 | 5.0 |
| Lung | 1.4 |
| Lung (fetal) | 3.4 |
| Lung ca. (small cell) LX-1 | 2.8 |
| Lung ca. (small cell) NCI-H69 | 7.1 |
| Lung ca. (s. cell var.) SHP-77 | 3.1 |
| Lung ca. (large cell)NCI-H460 | 0.3 |
| Lung ca. (non-sm. cell) A549 | 0.7 |
| Lung ca. (non-s. cell) NCI-H23 | 0.6 |
| Lung ca. (non-s. cell) HOP-62 | 2.8 |
| Lung ca. (non-s. cl) NCI-H522 | 0.4 |
| Lung ca. (squam.) SW 900 | 0.9 |
| Lung ca. (squam.) NCI-H596 | 3.4 |
| Mammary gland | 9.9 |
| Breast ca.* (pl. ef) MCF-7 | 13.3 |
| Breast ca.* (pl. ef) MDA-MB-231 | 3.2 |
| Breast ca.* (pl. ef) T47D | 12.2 |
| Breast ca. BT-549 | 1.6 |
| Breast ca. MDA-N | 3.9 |
| Ovary | 10.4 |
| Ovarian ca. OVCAR-3 | 4.5 |
| Ovarian ca. OVCAR-4 | 2.6 |
| Ovarian ca. OVCAR-5 | 3.7 |
| Ovarian ca. OVCAR-8 | 5.3 |
| Ovarian ca. IGROV-1 | 0.7 |
| Ovarian ca.* (ascites) SK-OV-3 | 1.0 |
| Uterus | 1.2 |
| Placenta | 0.2 |
| Prostate | 14.5 |
| Prostate ca.* (bone met)PC-3 | 1.1 |
| Testis | 2.3 |
| Melanoma Hs688(A).T | 0.1 |
| Melanoma* (met) Hs688(B).T | 0.6 |
| Melanoma UACC-62 | 0.0 |
| Melanoma M14 | 2.3 |
| Melanoma LOX IMVI | 0.2 |
| Melanoma* (met) SK-MEL-5 | 0.1 |
| Adipose | 1.0 |

Column A - Rel. Exp. (%) Ag2868, Run 162011291

TABLE GD
Panel 2D

| Tissue Name | A |
|---|---|
| Normal Colon | 43.8 |
| CC Well to Mod Diff (ODO3866) | 5.9 |
| CC Margin (ODO3866) | 8.9 |
| CC Gr.2 rectosigmoid (ODO3868) | 19.1 |
| CC Margin (ODO3868) | 1.4 |
| CC Mod Diff (ODO3920) | 34.2 |
| CC Margin (ODO3920) | 15.7 |
| CC Gr.2 ascend colon (ODO3921) | 38.4 |
| CC Margin (ODO3921) | 16.0 |
| CC from Partial Hepatectomy (ODO4309) Mets | 28.7 |
| Liver Margin (ODO4309) | 100.0 |
| Colon mets to lung (OD04451-01) | 6.7 |
| Lung Margin (OD04451-02) | 0.8 |
| Normal Prostate 6546-1 | 4.1 |
| Prostate Cancer (OD04410) | 20.3 |
| Prostate Margin (OD04410) | 16.3 |
| Prostate Cancer (OD04720-01) | 8.4 |
| Prostate Margin (OD04720-02) | 11.2 |
| Normal Lung 061010 | 6.7 |
| Lung Met to Muscle (ODO4286) | 0.5 |
| Muscle Margin (ODO4286) | 0.3 |
| Lung Malignant Cancer (OD03126) | 10.4 |
| Lung Margin (OD03126) | 4.1 |
| Lung Cancer (OD04404) | 20.9 |
| Lung Margin (OD04404) | 1.4 |
| Lung Cancer (OD04565) | 3.7 |
| Lung Margin (OD04565) | 1.3 |
| Lung Cancer (OD04237-01) | 14.2 |
| Lung Margin (OD04237-02) | 1.0 |
| Ocular Mel Met to Liver (ODO4310) | 6.8 |
| Liver Margin (ODO4310) | 58.6 |
| Melanoma Mets to Lung (OD04321) | 5.7 |
| Lung Margin (OD04321) | 2.9 |
| Normal Kidney | 30.1 |
| Kidney Ca, Nuclear grade 2 (OD04338) | 42.9 |
| Kidney Margin (OD04338) | 17.6 |
| Kidney Ca Nuclear grade 1/2 (OD04339) | 7.3 |
| Kidney Margin (OD04339) | 14.7 |
| Kidney Ca, Clear cell type (OD04340) | 0.3 |
| Kidney Margin (OD04340) | 14.1 |
| Kidney Ca, Nuclear grade 3 (OD04348) | 0.2 |
| Kidney Margin (OD04348) | 7.3 |
| Kidney Cancer (OD04622-01) | 9.3 |
| Kidney Margin (OD04622-03) | 4.0 |
| Kidney Cancer (OD04450-01) | 10.7 |
| Kidney Margin (OD04450-03) | 11.7 |
| Kidney Cancer 8120607 | 2.4 |
| Kidney Margin 8120608 | 9.7 |
| Kidney Cancer 8120613 | 31.9 |
| Kidney Margin 8120614 | 13.8 |
| Kidney Cancer 9010320 | 4.9 |
| Kidney Margin 9010321 | 17.1 |
| Normal Uterus | 0.3 |
| Uterus Cancer 064011 | 3.4 |
| Normal Thyroid | 8.0 |
| Thyroid Cancer 064010 | 7.5 |
| Thyroid Cancer A302152 | 6.8 |
| Thyroid Margin A302153 | 8.2 |
| Normal Breast | 4.6 |
| Breast Cancer (OD04566) | 12.1 |
| Breast Cancer (OD04590-01) | 22.1 |
| Breast Cancer Mets (OD04590-03) | 22.7 |
| Breast Cancer Metastasis (OD04655-05) | 23.8 |
| Breast Cancer 064006 | 4.8 |
| Breast Cancer 1024 | 41.2 |
| Breast Cancer 9100266 | 16.0 |
| Breast Margin 9100265 | 6.9 |
| Breast Cancer A209073 | 5.5 |
| Breast Margin A209073 | 7.8 |
| Normal Liver | 55.5 |
| Liver Cancer 064003 | 17.7 |
| Liver Cancer 1025 | 70.7 |
| Liver Cancer 1026 | 20.3 |
| Liver Cancer 6004-T | 90.1 |
| Liver Tissue 6004-N | 5.2 |
| Liver Cancer 6005-T | 17.2 |
| Liver Tissue 6005-N | 32.5 |
| Normal Bladder | 5.4 |
| Bladder Cancer 1023 | 2.8 |
| Bladder Cancer A302173 | 2.5 |
| Bladder Cancer (OD04718-01) | 7.2 |
| Bladder Normal Adjacent (OD04718-03) | 1.4 |
| Normal Ovary | 1.5 |
| Ovarian Cancer 064008 | 9.6 |
| Ovarian Cancer (OD04768-07) | 1.4 |
| Ovary Margin (OD04768-08) | 0.2 |
| Normal Stomach | 4.8 |
| Gastric Cancer 9060358 | 0.6 |
| Stomach Margin 9060359 | 5.0 |
| Gastric Cancer 9060395 | 7.9 |
| Stomach Margin 9060394 | 6.8 |
| Gastric Cancer 9060397 | 16.8 |
| Stomach Margin 9060396 | 3.4 |
| Gastric Cancer 064005 | 8.0 |

Column A - Rel. Exp. (%) Ag2868, Run 162011370

TABLE GE
Panel 4D

| Tissue Name | A |
|---|---|
| Secondary Th1 act | 48.0 |
| Secondary Th2 act | 40.9 |
| Secondary Tr1 act | 55.1 |
| Secondary Th1 rest | 3.1 |
| Secondary Th2 rest | 7.8 |
| Secondary Tr1 rest | 12.5 |
| Primary Th1 act | 64.6 |
| Primary Th2 act | 52.9 |
| Primary Tr1 act | 88.3 |
| Primary Th1 rest | 54.0 |
| Primary Th2 rest | 30.6 |
| Primary Tr1 rest | 100.0 |
| CD45RA CD4 lymphocyte act | 17.0 |
| CD45RO CD4 lymphocyte act | 33.0 |
| CD8 lymphocyte act | 30.8 |
| Secondary CD8 lymphocyte rest | 30.8 |
| Secondary CD8 lymphocyte act | 16.4 |
| CD4 lymphocyte none | 3.5 |
| 2ry Th1/Th2/Tr1 anti-CD95 CH11 | 9.9 |
| LAK cells rest | 10.2 |
| LAK cells IL-2 | 24.5 |
| LAK cells IL-2 + IL-12 | 30.4 |
| LAK cells IL-2 + IFN gamma | 31.4 |
| LAK cells IL-2 + IL-18 | 33.0 |
| LAK cells PMA/ionomycin | 1.8 |
| NK Cells IL-2 rest | 14.5 |
| Two Way MLR 3 day | 6.4 |
| Two Way MLR 5 day | 13.6 |
| Two Way MLR 7 day | 11.7 |
| PBMC rest | 2.0 |
| PBMC PWM | 43.8 |
| PBMC PHA-L | 22.7 |
| Ramos (B cell) none | 23.7 |
| Ramos (B cell) ionomycin | 62.9 |
| B lymphocytes PWM | 76.8 |
| B lymphocytes CD40L and IL-4 | 26.6 |
| EOL-1 dbcAMP | 27.2 |
| EOL-1 dbcAMP PMA/ionomycin | 12.3 |
| Dendritic cells none | 8.1 |
| Dendritic cells LPS | 3.1 |
| Dendritic cells anti-CD40 | 5.9 |
| Monocytes rest | 1.2 |
| Monocytes LPS | 0.9 |
| Macrophages rest | 15.1 |
| Macrophages LPS | 1.4 |
| HUVEC none | 1.7 |
| HUVEC starved | 1.8 |
| HUVEC IL-1beta | 0.1 |
| HUVEC IFN gamma | 2.7 |
| HUVEC TNF alpha + IFN gamma | 0.3 |
| HUVEC TNF alpha + IL4 | 1.1 |
| HUVEC IL-11 | 2.6 |
| Lung Microvascular EC none | 1.9 |
| Lung Microvascular EC TNFalpha + IL-1beta | 1.8 |
| Microvascular Dermal EC none | 1.0 |
| Microsvasular Dermal EC TNFalpha + IL-1beta | 1.4 |
| Bronchial epithelium TNFalpha + IL1beta | 1.1 |
| Small airway epithelium none | 3.8 |
| Small airway epithelium TNFalpha + IL-1beta | 14.2 |
| Coronery artery SMC rest | 0.3 |
| Coronery artery SMC TNFalpha + IL-1beta | 0.0 |
| Astrocytes rest | 3.0 |
| Astrocytes TNFalpha + IL-1beta | 1.7 |
| KU-812 (Basophil) rest | 25.3 |
| KU-812 (Basophil) PMA/ionomycin | 50.0 |
| CCD1106 (Keratinocytes) none | 12.7 |
| CCD1106 (Keratinocytes) TNFalpha + IL-1beta | 0.5 |
| Liver cirrhosis | 3.8 |
| Lupus kidney | 2.0 |
| NCI-H292 none | 31.4 |
| NCI-H292 IL-4 | 36.1 |
| NCI-H292 IL-9 | 41.8 |
| NCI-H292 IL-13 | 25.5 |
| NCI-H292 IFN gamma | 23.7 |
| HPAEC none | 1.0 |
| HPAEC TNF alpha + IL-1 beta | 0.2 |
| Lung fibroblast none | 1.5 |
| Lung fibroblast TNF alpha + IL-1 beta | 0.3 |
| Lung fibroblast IL-4 | 1.0 |
| Lung fibroblast IL-9 | 1.9 |
| Lung fibroblast IL-13 | 0.7 |
| Lung fibroblast IFN gamma | 0.5 |
| Dermal fibroblast CCD1070 rest | 1.8 |
| Dermal fibroblast CCD1070 TNF alpha | 31.6 |
| Dermal fibroblast CCD1070 IL-1 beta | 2.3 |
| Dermal fibroblast IFN gamma | 0.2 |
| Dermal fibroblast IL-4 | 2.6 |
| IBD Colitis 2 | 0.6 |
| IBD Crohn's | 3.0 |
| Colon | 31.6 |
| Lung | 2.9 |
| Thymus | 29.5 |
| Kidney | 11.9 |

Column A - Rel. Exp. (%) Ag2868, Run 159776784

TABLE GF
Panel 5 Islet

| Tissue Name | A |
|---|---|
| 97457_Patient-02go_adipose | 1.6 |
| 97476_Patient-07sk_skeletal muscle | 0.0 |
| 97477_Patient-07ut_uterus | 3.5 |
| 97478_Patient-07p1_placenta | 9.4 |
| 99167_Bayer Patient 1 | 12.2 |
| 97482_Patient-08ut_uterus | 0.7 |
| 97483_Patient-08p1_placenta | 4.0 |
| 97486_Patient-09sk_skeletal muscle | 0.0 |
| 97487_Patient-09ut_uterus | 4.0 |
| 97488_Patient-09p1_placenta | 9.8 |
| 97492_Patient-10ut_uterus | 6.6 |
| 97493_Patient-10p1_placenta | 6.1 |
| 97495_Patient-11go_adipose | 7.5 |
| 97496_Patient-11sk_skeletal muscle | 12.5 |
| 97497_Patient-11ut_uterus | 3.9 |
| 97498_Patient-11p1_placenta | 5.0 |
| 97500_Patient-12go_adipose | 13.8 |
| 97501_Patient-12sk_skeletal muscle | 20.6 |
| 97502_Patient-12ut_uterus | 1.3 |
| 97503_Patient-12p1_placenta | 2.1 |
| 94721_Donor2 U- A_Mesenchymal Stem Cells | 2.9 |
| 94722_Donor 2 U - B_Mesenchymal Stem Cells | 1.5 |
| 94723_Donor 2 U - C_Mesenchymal Stem Cells | 3.3 |
| 94709_Donor 2 AM - A_adipose | 6.4 |
| 94710_Donor 2 AM - B_adipose | 0.0 |
| 94711_Donor 2 AM - C_adipose | 0.0 |
| 94712_Donor 2 AD - A_adipose | 4.0 |
| 94713_Donor 2 AD - B_adipose | 2.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 3.9 |
| 94731_Donor 3 AM - B_adipose | 1.7 |
| 94732_Donor 3 AM - C_adipose | 4.1 |
| 94733_Donor 3 AD - A_adipose | 1.2 |
| 94734_Donor 3 AD - B_adipose | 2.0 |
| 94735_Donor 3 AD - C_adipose | 3.7 |
| 77138_Liver_HepG2untreated | 85.3 |
| 73556_Heart_Cardiac stromal cells (primary) | 0.0 |
| 81735_Small Intestine | 58.2 |
| 72409_Kidney_Proximal Convoluted Tubule | 11.6 |
| 82685_Small intestine_Duodenum | 58.2 |
| 90650_Adrenal_Adrenocortical adenoma | 5.4 |
| 72410_Kidney_HRCE | 100.0 |
| 72411_Kidney_HRE | 35.1 |
| 73139_Uterus_Uterine smooth muscle cells | 0.0 |

Column A - Rel. Exp. (%) Ag2868, Run 233071460

CNS_neurodegeneration_v1.0 Summary: Data obtained from RTQPCR assays performed for this panel was analyized using multivariate analysis (ANOVA). The multivariate results obtained using Ag2868 indicates this gene is found to be down-regulated in the temporal cortex of Alzheimer's disease patients. Therefore, up-regulation of this gene or its protein product, or treatment with specific agonists for this receptor may be of use in reversing the dementia/memory loss associated with this disease and neuronal death. This gene encodes for D-beta-hydroxybutyrate dehydrogenase homolog. D-beta-hydroxybutyrate dehydrogenase function is also controlled at the translational, post-translational and catalytic levels. (Kante A, et al., 1990, Biochim Biophys Acta 1033(3):291-7). Please see Panel 1.3D for additional discussion of role of this gene in the central nervous system.

Panel 1.3D Summary: Ag2868 Expression of this gene is highest in the cerebral cortex (CT=27.6). The expression of this gene in multiple brain regions is consistent with a published role for this gene in CNS energetic processes. This gene encodes a hydroxybutyrate dehydrogenase homolog. D-beta-hydroxybutyrate protects neurons in models of Alzheimer's and Parkinson's disease. Other enzymes, such as amyloid beta-peptide-binding alcohol dehydrogenase, which have been shown to possess D-beta-hydroxybutyrate dehydrogenase activity, contribute to the protective response to metabolic stress, especially in the setting of ischemia (Kashiwaya Y, et al. Proc Natl Acad Sci USA May 9, 2000;97(10):5440-4; Du Yan S, et al. J Biol Chem Sep. 1, 2000;275(35):27100-9). Since this protein encoded by this gene processes D-beta-hydroxybutyrate to provide a neuronal energy source, activators of the protein encoded by this gene may be useful in treating and protecting the CNS of Alzheimer's and Parkinson's disease patients, as well as stroke.

Overall, expression of this gene appears to be largely associated with normal tissues when compared to cancer cell lines. Thus, therapeutic modulation of this gene, through the use of small molecule drugs, antibodies or protein therapeutics might be of benefit in the treatment of cancer.

This gene is also moderately expressed in a variety of metabolic tissues, including pancreas, adrenal, thyroid, pituitary, adult and fetal heart, adult and fetal skeletal muscle, adult and fetal liver and adipose. Mutations in the hydroxybutyrate dehydrogenase enzyme are associated with hypoglycemia and cardiac arrest. Activators of this enzyme could be drug targets for obesity because increased fatty acid oxidation may prevent the incorporation of fatty acids into triglylcerides, thus decreasing adipose mass.

Panel 2D Summary: Ag2868 The expression of this gene appears to be highest in a sample derived from normal liver tissue adjacent to a metastatic colon cancer (CT=25.9). In addition, there appears to be substantial expression associated with malignant liver tissue when compared to their associated normal adjacent tissue. Thus, therapeutic modulation of this gene, through the use of small molecule drugs, antibodies or protein therapeutics might be of benefit in the treatment of liver cancer.

Panel 4D Summary: Ag2868 This gene is expressed primarily in activated leukocytes, especially in T cells and B cells (CTs=27-30). It is also expressed in NCI-H292 cells and in TNF alpha treated dermal fibroblasts. The protein encoded for by this trancript has homology to hydroxybutyrate dehydrogenase, a protein that has been found in lymphocytes (Curi R, Williams J F, Newsholme E A., 1989, Pyruvate metabolism by lymphocytes: evidence for an additional ketogenic tissue. Biochem Int 19(4):755-67). Thus, the protein encoded for by this transcript may be important for cellular responses to inflammatory/activating stimuli. Therefore, therapeutics designed with the protein encoded for by this transcript could be used for the treatment of inflammatory diseases such as asthma, emphysema, COPD, arthritis, IBD and psoriasis.

Panel 5 Islet Summary: Ag2868 Expression of this gene is highest a in kidney cell line (CT=32.8). In addition low expression of this gene is also seen in a liver cancer cell line, and small intestine. Please see panel 1.3D for further discussion of this gene.

H. CG56151-01: Glucose Transporter Type2

Expression of gene CG56151-01 was assessed using the primer-probe set Ag1681, described in Table HA. Results of the RTQ-PCR runs are shown in Tables HB, HC, HD, HE, HF and HG.

TABLE HA
Probe Name Ag1681

| | | | | |
|---|---|---|---|---|
| | | | Start | SEQ ID |
| Primers | Sequences | Length | Position | No |

| | | | | |
|---|---|---|---|---|
| Forward | 5′-ggacttctgtggaccttatgtg-3′ | 22 | 1412 | 119 |
| Probe | TET-5′-ttttcctctttgctggagtgctcctg-3′-TAMRA | 26 | 1435 | 120 |
| Reverse | 5′-ttcctttggtttctggaacttt-3′ | 22 | 1485 | 121 |

TABLE HB
General screening panel_v1.4

| Tissue Name | A |
|---|---|
| Adipose | 0.0 |
| Melanoma* Hs688(A).T | 0.0 |
| Melanoma* Hs688(B).T | 0.0 |
| Melanoma* M14 | 0.2 |
| Melanoma* LOXIMVI | 0.2 |
| Melanoma* SK-MEL-5 | 0.0 |
| Squamous cell carcinoma SCC-4 | 0.0 |
| Testis Pool | 0.4 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.4 |
| Ovarian ca. SK-OV-3 | 0.0 |
| Ovarian ca. OVCAR-4 | 0.0 |
| Ovarian ca. OVCAR-5 | 0.1 |
| Ovarian ca. IGROV-1 | 0.0 |
| Ovarian ca. OVCAR-8 | 0.1 |
| Ovary | 0.2 |
| Breast ca. MCF-7 | 0.2 |
| Breast ca. MDA-MB-231 | 0.0 |
| Breast ca. BT 549 | 0.0 |
| Breast ca. T47D | 0.1 |
| Breast ca. MDA-N | 0.2 |
| Breast Pool | 0.1 |
| Trachea | 0.0 |
| Lung | 0.1 |
| Fetal Lung | 1.2 |
| Lung ca. NCI-N417 | 0.0 |
| Lung ca. LX-1 | 0.1 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.2 |
| Lung ca. NCI-H460 | 0.0 |
| Lung ca. HOP-62 | 0.6 |
| Lung ca. NCI-H522 | 0.0 |
| Liver | 23.2 |
| Fetal Liver | 100.0 |
| Liver ca. HepG2 | 7.4 |
| Kidney Pool | 0.1 |
| Fetal Kidney | 1.9 |
| Renal ca. 786-0 | 0.6 |
| Renal ca. A498 | 0.1 |
| Renal ca. ACHN | 4.8 |
| Renal ca. UO-31 | 0.1 |
| Renal ca. TK-10 | 0.1 |
| Bladder | 0.4 |
| Gastric ca. (liver met.) NCI-N87 | 0.7 |
| Gastric ca. KATO III | 0.1 |
| Colon ca. SW-948 | 0.0 |
| Colon ca. SW480 | 0.2 |
| Colon ca.* (SW480 met) SW620 | 0.0 |
| Colon ca. HT29 | 0.1 |
| Colon ca. HCT-116 | 0.1 |
| Colon ca. CaCo-2 | 3.7 |
| Colon cancer tissue | 0.0 |
| Colon ca. SW1116 | 0.0 |
| Colon ca. Colo-205 | 0.1 |
| Colon ca. SW-48 | 0.0 |
| Colon Pool | 0.0 |
| Small Intestine Pool | 0.0 |
| Stomach Pool | 0.2 |
| Bone Marrow Pool | 0.0 |
| Fetal Heart | 0.0 |
| Heart Pool | 0.1 |
| Lymph Node Pool | 0.1 |
| Fetal Skeletal Muscle | 0.1 |
| Skeletal Muscle Pool | 0.0 |
| Spleen Pool | 0.1 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.1 |
| CNS cancer (glio) SNB-19 | 0.0 |
| CNS cancer (glio) SF-295 | 0.0 |
| Brain (Amygdala) Pool | 0.2 |
| Brain (cerebellum) | 0.0 |
| Brain (fetal) | 0.0 |
| Brain (Hippocampus) Pool | 0.1 |
| Cerebral Cortex Pool | 0.2 |
| Brain (Substantia nigra) Pool | 0.4 |
| Brain (Thalamus) Pool | 0.1 |
| Brain (whole) | 0.4 |
| Spinal Cord Pool | 0.3 |
| Adrenal Gland | 0.1 |
| Pituitary gland Pool | 0.1 |
| Salivary Gland | 0.0 |
| Thyroid (female) | 0.0 |
| Pancreatic ca. CAPAN2 | 0.5 |
| Pancreas Pool | 0.5 |

Column A - Rel. Exp. (%) Ag1681, Run 208016706

TABLE HC
Human Metabolic

| Tissue Name | A |
|---|---|
| 137857_psoas-AA.M.Diab.-hi BMI-6 | 0.0 |
| 135760_psoas-HI.M.Diab.-hi BMI-21 | 0.7 |
| 134827_psoas-CC.M.Diab.-hi BMI-4 | 0.0 |
| 137860_psoas-AA.M.Diab.-med BMI-8 | 0.0 |
| 137834_psoas-CC.M.Diab.-med BMI-2 | 0.0 |
| 137828_psoas-CC.M.Diab.-med BMI-1 | 0.0 |
| 135763_psoas-HI.M.Diab.-med BMI-23 | 0.0 |
| 142740_psoas-AS.M.Diab.-low BMI-20 | 0.0 |
| 134834_psoas-AA.M.Diab.-low BMI-17 | 0.1 |
| 137850_psoas-AS.M.Norm-hi BMI-34 | 0.0 |
| 135769_psoas-HI.M.Norm-hi BMI-31 | 0.0 |
| 135766_psoas-AA.M.Norm-hi BMI-25 | 0.0 |
| 142746_psoas-AA.M.Norm-med BMI-37 | 0.0 |
| 142745_psoas-HI.M.Norm-med BMI-35 | 0.0 |
| 137855_psoas-AA.M.Norm-med BMI-47 | 0.0 |
| 137844_psoas-CC.M.Norm-med BMI-26 | 0.0 |
| 142742_psoas-CC.M.Norm-low BMI-40 | 0.0 |
| 137873_psoas-AS.M.Norm-low BMI-28 | 0.0 |
| 137853_psoas-HI.M.Norm-low BMI-41 | 0.0 |
| 135775_psoas-CC.M.Norm-low BMI-39 | 0.0 |
| 137858_diaphragm-AA.M.Diab.-hi BMI-6 | 0.0 |
| 135772_diaphragm-AS.M.Diab-hi BMI-9 | 0.0 |
| 135761_diaphragm-HI.M.Diab.-hi BMI-21 | 0.1 |
| 134828_diaphragm-CC.M.Diab.-hi BMI-4 | 0.0 |
| 137835_diaphragm-CC.M.Diab.-med BMI-2 | 0.0 |
| 135764_diaphragm-HI.M.Diab.-med BMI-23 | 0.0 |
| 134835_diaphragm-AA.M.Diab.-low BMI-17 | 0.0 |
| 142738_diaphragm-CC.M.Norm-hi BMI-29 | 0.0 |
| 139517_diaphragm-AS.M.Norm-hi BMI-34 | 0.0 |
| 137848_diaphragm-HI.M.Norm-hi BMI-31 | 0.0 |
| 137843_diaphragm-AA.M.Norm-hi BMI-25 | 0.0 |
| 137879_diaphragm-AA.M.Norm-med BMI-47 | 0.0 |
| 137872_diaphragm-CC.M.Norm-med BMI-26 | 0.0 |
| 135773_diaphragm-HI.M.Norm-med BMI-35 | 0.0 |
| 139542_diaphragm-HI.M.Norm-low BMI-41 | 0.0 |
| 137877_diaphragm-CC.M.Norm-low BMI-39 | 0.0 |
| 137874_diaphragm-AS.M.Norm-low BMI-28 | 0.0 |
| 141340_subQadipose-AA.M.Diab.-hi BMI-6 | 0.0 |
| 137836_subQadipose-HI.M.Diab.-hi BMI-21 | 0.0 |
| 135771_subQadipose-AS.M.Diab-hi BMI-9 | 0.0 |
| 141329_subQadipose-AA.M.Diab-medbmi-8 | 0.0 |
| 137862_subQadipose-CC.M.Diab.-med BMI-1 | 0.0 |
| 135762_subQadipose-HI.M.Diab.-med BMI-23 | 0.0 |
| 141338_subQadipose-AS.M.Diab.-low BMI-20 | 0.0 |
| 139547_subQadipose-HI.M.Diab.-low BMI-22 | 0.1 |
| 135757_subQadipose-CC.M.Diab.-low BMI-13 | 0.0 |
| 134832_subQadipose-AA.M.Diab.-low BMI-17 | 0.0 |
| 141332_subQadipose-HI.M.Norm-hi BMI-31 | 0.0 |
| 135767_subQadipose-CC.M.Norm-hi BMI-29 | 0.0 |
| 135765_subQadipose-AS.M.Norm-hi BMI-34 | 0.0 |
| 141339_subQadipose-HI.M.Norm-med BMI-35 | 0.0 |
| 141334_subQadipose-CC.M.Norm-med BMI-26 | 0.0 |
| 139544_subQadipose-AA.M.Norm-med BMI-47 | 0.0 |
| 137875_subQadipose-AA.M.Norm-med BMI-37 | 0.0 |
| 141331_subQadipose-AS.M.Norm-low BMI-28 | 0.0 |
| 137878_subQadipose-HI.M.Norm-low BMI-41 | 0.0 |
| 137876_subQadipose-CC.M.Norm-low BMI-39 | 0.0 |
| 137859_vis.adipose-AA.M.Diab.-hi BMI-6 | 0.0 |
| 135770_vis.adipose-AS.M.Diab-hi BMI-9 | 0.0 |
| 135759_vis.adipose-HI.M.Diab.-hi BMI-21 | 0.0 |
| 143502_vis.adipose-CC.M.Diab.-med BMI-2 | 0.0 |
| 139510_vis.adipose-AA.M.Diab.-med BMI-8 | 0.0 |
| 137861_vis.adipose-CC.M.Diab.-med-1 | 0.0 |
| 137839_vis.adipose-HI.M.Diab.-med BMI-23 | 0.0 |
| 139546_vis.adipose-HI.M.Diab.-low BMI-22 | 0.0 |
| 137831_vis.adipose-CC.M.Diab.-low BMI-13 | 0.0 |
| 139522_vis.adipose-HI.M.Norm-hi BMI-31 | 0.0 |
| 139516_vis.adipose-AS.M.Norm-hi BMI-34 | 0.0 |
| 137846_vis.adipose-CC.M.Norm-hi BMI-29 | 0.0 |
| 137841_vis.adipose-AA.M.Norm-hi BMI-25 | 0.0 |
| 139543_vis.adipose-AA.M.Norm-med BMI-47 | 0.0 |
| 139532_vis.adipose-AA.M.Norm-med BMI-37 | 0.0 |
| 139530_vis.adipose-HI.M.Norm-med BMI-35 | 0.0 |
| 139539_vis.adipose-HI.M.Norm-low BMI-41 | 0.0 |
| 139535_vis.adipose-CC.M.Norm-low BMI-40 | 0.1 |
| 137852_vis.adipose-CC.M.Norm-low BMI-39 | 0.0 |
| 135768_vis.adipose-AS.M.Norm-low BMI-28 | 0.0 |
| 141327_liver-CC.M.Diab.-hi BMI-4 | 0.2 |
| 139514_liver-HI.M.Diab.-hi BMI-21 | 3.7 |
| 139526_liver-CC.M.Diab.-med BMI-2 | 2.0 |
| 139511_liver-AA.M.Diab.-med BMI-8 | 1.4 |
| 137840_liver-HI.M.Diab.-med BMI-23 | 31.0 |
| 137827_liver-CC.M.Diab.-med BMI-1 | 27.2 |
| 137838_liver-HI.M.Diab.-low BMI-22 | 28.9 |
| 135758_liver-CC.M.Diab.-low BMI-13 | 23.5 |
| 139519_liver-CC.M.Norm-hi BMI-29 | 7.9 |
| 139518_liver-AA.M.Norm-hi BMI-25 | 16.2 |
| 137849_liver-AS.M.Norm-hi BMI-34 | 72.2 |
| 137847_liver-HI.M.Norm-hi BMI-31 | 26.2 |
| 142741_liver-AA.M.Norm-med BMI-37 | 100.0 |
| 141341_liver-HI.M.Norm-med BMI-35 | 8.6 |
| 141335_liver-CC.M.Norm-med BMI-26 | 1.7 |
| 139540_liver-HI.M.Norm-low BMI-41 | 1.6 |
| 139534_liver-CC.M.Norm-low BMI-39 | 7.4 |
| 139521_liver-AS.M.Norm-low BMI-28 | 15.2 |
| 141328_pancreas-CC.M.Diab.-hi BMI-4 | 0.0 |
| 139525_pancreas-AS.M.Diab.-hi BMI-9 | 0.0 |
| 137856_pancreas-AA.M.Diab.-hi BMI-6 | 0.1 |
| 137837_pancreas-HI.M.Diab.-hi BMI-21 | 0.6 |
| 141337_pancreas-CC.M.Diab.-med BMI-2 | 0.0 |
| 139527_pancreas-CC.M.Diab.-med BMI-1 | 0.0 |
| 139515_pancreas-HI.M.Diab.-med BMI-23 | 0.0 |
| 139512_pancreas-AA.M.Diab.-med BMI-8 | 0.0 |
| 142739_pancreas-AS.M.Diab.-low BMI-20 | 0.0 |
| 139513_pancreas-CC.M.Diab.-low BMI-13 | 0.0 |
| 142743_pancreas-AA.M.Norm-hi BMI-25 | 0.0 |
| 139523_pancreas-HI.M.Norm-hi BMI-31 | 0.0 |
| 139520_pancreas-CC.M.Norm-hi BMI-29 | 0.0 |
| 142744_pancreas-HI.M.Norm-med BMI-35 | 0.0 |
| 139545_pancreas-AA.M.Norm-med BMI-47 | 0.0 |
| 13953l_pancreas-AA.M.Norm-med BMI-37 | 0.0 |
| 13787l_pancreas-CC.M.Norm-med BMI-26 | 0.7 |
| 139541_pancreas-Hi.M.Norm-low BMI-41 | 0.0 |
| 139537_pancreas-CC.M.Norm-low BMI-40 | 0.0 |
| 139533_pancreas-CC.M.Norm-low BMI-39 | 0.0 |
| 137845_pancreas-AS.M.Norm-low BMI-28 | 0.1 |
| 143530_small intestine-AA.M.Diab.-hi BMI-6 | 0.0 |
| 143529_small intestine-CC.M.Diab.-hi BMI-4 | 0.1 |
| 143538_small intestine-HI.M.Diab.-med BMI-23 | 0.0 |
| 143531_small intestine-AA.M.Diab.-med BMI-8 | 0.1 |
| 143528_small intestine-CC.M.Diab.-med BMI-2 | 0.0 |
| 143537_small intestine-HI.M.Diab.-low BMI-22 | 0.0 |
| 143535_small intestine-AS.M.Diab.-low BMI-20 | 0.0 |
| 143534_small intestine-AA.M.Diab.-low BMI-17 | 0.0 |
| 143544_small intestine-AS.M.Norm-hi BMI-34 | 0.1 |
| 143543_small intestine-HI.M.Norm-hi BMI-31 | 1.4 |
| 143542_small intestine-CC.M.Norm-hi BMI-29 | 0.0 |
| 143539_small intestine-AA.M.Norm-hi BMI-25 | 0.0 |
| 143548_small intestine-AA.M.Norm-med BMI-47 | 0.1 |
| 143547_small intestine-AA.M.Norm-med BMI-37 | 0.1 |
| 143540_small intestine-CC.M.Norm-med BMI-26 | 0.0 |
| 143550_small intestine-CC.M.Norm-low BMI-40 | 0.0 |
| 143549_small intestine-CC.M.Norm-low BMI-39 | 0.0 |
| 143546_small intestine-HI.M.Norm-low BMI-41 | 0.1 |
| 143525_hypothalamus-HI.M.Diab.-hi BMI-21 | 0.0 |
| 143515_hypothalamus-CC.M.Diab.-hi BMI-4 | 0.0 |
| 143513_hypothalamus-AA.M.Diab.-hi BMI-6 | 0.2 |
| 143507_hypothalamus-AS.M.Diab.-hi BMI-9 | 0.1 |
| 143506_hypothalamus-CC.M.Diab.-med BMI-1 | 0.2 |
| 143505_hypothalamus-HI.M.Diab.-med BMI-23 | 0.1 |
| 143509_hypothalamus-AA.M.Diab.-low BMI-17 | 0.1 |
| 143508_hypothalamus-CC.M.Diab.-low BMI-13 | 0.1 |
| 143503_hypothalamus-AS.M.Diab.-low BMI-20 | 0.1 |
| 143522_hypothalamus-HI.M.Norm-hi BMI-31 | 0.0 |
| 143516_hypothalamus-AS.M.Norm-hi BMI-34 | 0.0 |
| 143511_hypothalamus-CC.M.Norm-hi BMI-29 | 0.2 |
| 143504_hypothalamus-AA.M.Norm-hi BMI-25 | 0.1 |
| 143517_hypothalamus-AA.M.Norm-med BMI-47 | 0.1 |
| 143514_hypothalamus-HI.M.Norm-med BMI-35 | 0.0 |
| 143521_hypothalamus-AS.M.Norm-low BMI-28 | 0.0 |
| 143512_hypothalamus-CC.M.Norm-low BMI-40 | 0.0 |
| 145454_Patient-25pl (CC.Diab.low BMI.no insulin) | 0.0 |
| 110916_Patient-18pl (HI.Diab.obese.no insulin) | 0.0 |
| 110913_Patient-18go (HI.Diab.obese.no insulin) | 0.0 |
| 110911_Patient-17pl (CC.Diab.low BMI.no insulin) | 0.0 |
| 110908_Patient-17go (CC.Diab.low BMI.no insulin) | 0.0 |
| 100752_Patient-15sk (CC.Diab.obese.no insulin) | 0.0 |
| 97828_Patient-13pl (CC.Diab.overwt.no insulin) | 0.1 |
| 160114_Patient27-ut (CC.Diab.obese.insulin) | 0.0 |
| 160113_Patient27-pl (CC.Diab.obese.insulin) | 0.0 |
| 160112_Patient27-sk (CC.Diab.obese.insulin) | 0.0 |
| 160111_Patient27-go (CC.Diab.obese.insulin) | 0.1 |
| 145461_Patient-26sk (CC.Diab.obese.insulin) | 0.0 |
| 145441_Patient-22sk (CC.Diab.low BMI.insulin) | 0.1 |
| 145438_Patient-22pl (CC.Diab.low BMI.insulin) | 0.0 |
| 145427_Patient-20pl (CC.Diab.overwt.insulin) | 0.1 |
| 97503_Patient-12pl (CC.Diab.unknown BMI.insulin) | 0.0 |
| 145443_Patient-23pl (CC.Non-diab.overwt) | 0.0 |
| 145435_Patient-21pl (CC.Non-diab.overwt) | 0.0 |
| 110921_Patient-19pl (CC.Non-diab.low BMI) | 0.0 |
| 110918_Patient-19go (CC.Non-diab.low BMI) | 0.1 |
| 97481_Patient-08sk (CC.Non-diab.obese) | 0.0 |
| 97478_Patient-07pl (CC.Non-diab.obese) | 0.0 |
| 160117_Human Islets-male, obese | 0.5 |
| 145474_PANC1 (pancreas carcinoma) 1 | 0.7 |
| 154911_Capan2 (pancreas adenocarcinoma) | 4.4 |
| 141190_SW579 (thyroid carcinoma) | 0.0 |
| 145489_SK-N-MC (neuroblastoma) 1 | 0.0 |
| 145495_SK-N-SH (neuroblastoma) 1 | 0.5 |
| 145498_U87 MG (glioblastoma) 2 | 0.0 |
| 145484_HEp-2 (larynx carcinoma) 1 | 0.0 |
| 145479_A549 (lung carcinoma) | 0.0 |
| 145488_A427 (lung carcinoma) 2 | 0.0 |
| 145472_FHs 738Lu (normal lung) 1 | 0.1 |
| 141187_SKW6.4 (B lymphocytes) | 0.0 |
| 154644_IM-9 (immunoglobulin secreting lymphoblast) | 0.0 |
| 154645_MOLT-4 (acute lymphoblastic | 0.0 |
| leukemia derived from peripheral blood) | |
| 154648_U-937 (histiocystic lymphoma) | 0.1 |
| 154647_Daudi (Burkitt's lymphoma) | 0.1 |
| 145494_SK-MEL-2 (melanoma) 2 | 0.3 |
| 141176_A375 (melanoma) | 0.0 |
| 154642_SW 1353 (humerus chondrosarcoma) | 0.1 |
| 141179_HT-1080 (fibrosarcoma) | 0.0 |
| 145491_MG-63 (osteosarcoma) 1 | 0.0 |
| 141186_MCF7 (breast carcinoma) | 0.0 |
| 141193_T47D (breast carcinoma) | 0.1 |
| 154641_BT-20 (breast carcinoma) | 1.6 |
| 141175_293 (kidney transformed with adenovirus 5 DNA) | 0.0 |
| 141182_HUH hepatoma 1 | 0.6 |
| 141184_HUH7 hepatoma 1 | 0.8 |
| 145478_HT1376 (bladder carcinoma) | 0.0 |
| 14548l_SCaBER (bladder carcinoma) | 0.3 |
| 141192_SW620 (lymph node metastatsis, colon carcinoma) 2 | 0.1 |
| 141180_HT29 (colon carcinoma) 1 | 0.2 |
| 141188_SW480 (colon carcinoma) 1 | 0.2 |
| 154646_CAOV-3 (ovary adenocarcinoma) | 0.2 |
| 141194_HeLa (cervix carcinoma)- 2 | 0.0 |
| 145482_HeLa S3 (cervix carcinoma) 1 | 0.0 |
| 145486_DU145 (prostate carcinoma) | 0.0 |
| 154643_PC-3 (prostate adenocarcinoma) | 0.0 |
| 154649_HCT-8 (ileocecal adenocarcinoma) | 0.2 |

Column A - Rel. Exp. (%) Ag1681, Run 324668035

TABLE HD
Panel 1.3D

| Tissue Name | A |
|---|---|
| Liver adenocarcinoma | 0.0 |
| Pancreas | 1.5 |
| Pancreatic ca. CAPAN 2 | 0.3 |
| Adrenal gland | 0.1 |
| Thyroid | 0.0 |
| Salivary gland | 0.0 |
| Pituitary gland | 0.2 |
| Brain (fetal) | 0.0 |
| Brain (whole) | 0.0 |
| Brain (amygdala) | 0.0 |
| Brain (cerebellum) | 0.0 |
| Brain (hippocampus) | 0.1 |
| Brain (substantia nigra) | 0.0 |
| Brain (thalamus) | 0.0 |
| Cerebral Cortex | 0.1 |
| Spinal cord | 0.1 |
| glio/astro U87-MG | 0.2 |
| glio/astro U-118-MG | 0.0 |
| astrocytoma SW1783 | 0.2 |
| neuro*; met SK-N-AS | 0.1 |
| astrocytoma SF-539 | 0.2 |
| astrocytoma SNB-75 | 0.0 |
| glioma SNB-19 | 0.1 |
| glioma U251 | 0.0 |
| glioma SF-295 | 0.0 |
| Heart (fetal) | 0.0 |
| Heart | 0.0 |
| Skeletal muscle (fetal) | 0.0 |
| Skeletal muscle | 0.0 |
| Bone marrow | 0.0 |
| Thymus | 0.0 |
| Spleen | 0.0 |
| Lymph node | 0.0 |
| Colorectal | 0.3 |
| Stomach | 0.1 |
| Small intestine | 7.6 |
| Colon ca. SW480 | 0.3 |
| Colon ca.* SW620(SW480 met) | 0.0 |
| Colon ca. HT29 | 0.2 |
| Colon ca. HCT-116 | 0.0 |
| Colon ca. CaCo-2 | 8.8 |
| Colon ca. tissue (ODO3866) | 0.1 |
| Colon ca. HCC-2998 | 1.7 |
| Gastric ca.* (liver met) NCI-N87 | 0.8 |
| Bladder | 0.4 |
| Trachea | 0.0 |
| Kidney | 8.9 |
| Kidney (fetal) | 5.8 |
| Renal ca. 786-0 | 0.5 |
| Renal ca. A498 | 0.5 |
| Renal ca. RXF 393 | 0.1 |
| Renal ca. ACHN | 11.7 |
| Renal ca. UO-31 | 0.2 |
| Renal ca. TK-10 | 0.1 |
| Liver | 100.0 |
| Liver (fetal) | 99.3 |
| Liver ca. (hepatoblast) HepG2 | 22.2 |
| Lung | 0.0 |
| Lung (fetal) | 0.0 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.0 |
| Lung ca. (non-sm. cell) A549 | 0.1 |
| Lung ca. (non-s. cell) NCI-H23 | 0.7 |
| Lung ca. (non-s. cell) HOP-62 | 0.5 |
| Lung ca. (non-s. cl) NCI-H522 | 0.0 |
| Lung ca. (squam.) SW 900 | 0.1 |
| Lung ca. (squam.) NCI-H596 | 0.0 |
| Mammary gland | 0.0 |
| Breast ca.* (pl. ef) MCF-7 | 0.2 |
| Breast ca.* (pl. ef) MDA-MB-231 | 0.2 |
| Breast ca.* (pl. ef) T47D | 0.2 |
| Breast ca. BT-549 | 0.0 |
| Breast ca. MDA-N | 0.4 |
| Ovary | 0.1 |
| Ovarian ca. OVCAR-3 | 0.3 |
| Ovarian ca. OVCAR-4 | 0.1 |
| Ovarian ca. OVCAR-5 | 0.2 |
| Ovarian ca. OVCAR-8 | 0.2 |
| Ovarian ca. IGROV-1 | 0.0 |
| Ovarian ca.* (ascites) SK-OV-3 | 0.0 |
| Uterus | 0.0 |
| Placenta | 0.0 |
| Prostate | 0.0 |
| Prostate ca.* (bone met)PC-3 | 0.0 |
| Testis | 0.2 |
| Melanoma Hs688(A).T | 0.0 |
| Melanoma* (met) Hs688(B).T | 0.1 |
| Melanoma UACC-62 | 0.0 |
| Melanoma M14 | 0.2 |
| Melanoma LOX IMVI | 0.0 |
| Melanoma* (met) SK-MEL-5 | 0.0 |
| Adipose | 0.1 |

Column A - Rel. Exp. (%) Ag1681, Run 146581527

TABLE HE
Panel 2D

| Tissue Name | A |
|---|---|
| Normal Colon | 0.5 |
| CC Well to Mod Diff (ODO3866) | 0.0 |
| CC Margin (ODO3866) | 0.0 |
| CC Gr.2 rectosigmoid (ODO3868) | 0.0 |
| CC Margin (ODO3868) | 0.0 |
| CC Mod Diff (ODO3920) | 0.1 |
| CC Margin (ODO3920) | 0.0 |
| CC Gr.2 ascend colon (ODO3921) | 0.0 |
| CC Margin (ODO3921) | 0.0 |
| CC from Partial Hepatectomy (ODO4309) Mets | 6.6 |
| Liver Margin (ODO4309) | 100.0 |
| Colon mets to lung (OD04451-01) | 0.0 |
| Lung Margin (OD04451-02) | 0.0 |
| Normal Prostate 6546-1 | 0.0 |
| Prostate Cancer (OD04410) | 0.0 |
| Prostate Margin (OD04410) | 0.0 |
| Prostate Cancer (OD04720-01) | 0.0 |
| Prostate Margin (OD04720-02) | 0.0 |
| Normal Lung 061010 | 0.1 |
| Lung Met to Muscle (ODO4286) | 0.0 |
| Muscle Margin (ODO4286) | 0.0 |
| Lung Malignant Cancer (OD03126) | 0.0 |
| Lung Margin (OD03126) | 0.0 |
| Lung Cancer (OD04404) | 0.0 |
| Lung Margin (OD04404) | 0.0 |
| Lung Cancer (OD04565) | 0.0 |
| Lung Margin (OD04565) | 0.0 |
| Lung Cancer (OD04237-01) | 0.0 |
| Lung Margin (OD04237-02) | 0.0 |
| Ocular Mel Met to Liver (ODO4310) | 0.0 |
| Liver Margin (ODO4310) | 62.0 |
| Melanoma Mets to Lung (OD04321) | 0.0 |
| Lung Margin (OD04321) | 0.0 |
| Normal Kidney | 9.2 |
| Kidney Ca, Nuclear grade 2 (OD04338) | 1.0 |
| Kidney Margin (OD04338) | 1.5 |
| Kidney Ca Nuclear grade 1/2 (OD04339) | 0.0 |
| Kidney Margin (OD04339) | 12.9 |
| Kidney Ca, Clear cell type (OD04340) | 10.4 |
| Kidney Margin (OD04340) | 3.7 |
| Kidney Ca, Nuclear grade 3 (OD04348) | 0.3 |
| Kidney Margin (OD04348) | 2.1 |
| Kidney Cancer (OD04622-01) | 1.0 |
| Kidney Margin (OD04622-03) | 0.5 |
| Kidney Cancer (OD04450-01) | 0.4 |
| Kidney Margin (OD04450-03) | 2.2 |
| Kidney Cancer 8120607 | 0.0 |
| Kidney Margin 8120608 | 1.7 |
| Kidney Cancer 8120613 | 0.0 |
| Kidney Margin 8120614 | 2.2 |
| Kidney Cancer 9010320 | 0.1 |
| Kidney Margin 9010321 | 4.0 |
| Normal Uterus | 0.0 |
| Uterus Cancer 064011 | 0.0 |
| Normal Thyroid | 0.1 |
| Thyroid Cancer 064010 | 0.2 |
| Thyroid Cancer A302152 | 0.1 |
| Thyroid Margin A302153 | 0.1 |
| Normal Breast | 0.1 |
| Breast Cancer (OD04566) | 0.1 |
| Breast Cancer (OD04590-01) | 0.0 |
| Breast Cancer Mets (OD04590-03) | 0.1 |
| Breast Cancer Metastasis (OD04655-05) | 0.0 |
| Breast Cancer 064006 | 0.5 |
| Breast Cancer 1024 | 0.0 |
| Breast Cancer 9100266 | 0.0 |
| Breast Margin 9100265 | 0.0 |
| Breast Cancer A209073 | 0.1 |
| Breast Margin A209073 | 0.1 |
| Normal Liver | 86.5 |
| Liver Cancer 064003 | 23.5 |
| Liver Cancer 1025 | 39.8 |
| Liver Cancer 1026 | 13.6 |
| Liver Cancer 6004-T | 47.0 |
| Liver Tissue 6004-N | 8.1 |
| Liver Cancer 6005-T | 12.6 |
| Liver Tissue 6005-N | 14.1 |
| Normal Bladder | 0.3 |
| Bladder Cancer 1023 | 0.0 |
| Bladder Cancer A302173 | 0.0 |
| Bladder Cancer (OD04718-01) | 0.0 |
| Bladder Normal Adjacent (OD04718-03) | 0.0 |
| Normal Ovary | 0.0 |
| Ovarian Cancer 064008 | 0.0 |
| Ovarian Cancer (OD04768-07) | 0.8 |
| Ovary Margin (OD04768-08) | 0.0 |
| Normal Stomach | 0.0 |
| Gastric Cancer 9060358 | 0.0 |
| Stomach Margin 9060359 | 0.0 |
| Gastric Cancer 9060395 | 0.0 |
| Stomach Margin 9060394 | 0.3 |
| Gastric Cancer 9060397 | 0.0 |
| Stomach Margin 9060396 | 0.0 |
| Gastric Cancer 064005 | 0.4 |

Column A - Rel. Exp. (%) Ag1681, Run 148168295

TABLE HF
Panel 5 Islet

| Tissue Name | A |
|---|---|
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 0.7 |
| 99167_Bayer Patient 1 | 1.3 |
| 97482_Patient-08ut_uterus | 0.0 |
| 97483_Patient-08pl_placenta | 0.0 |
| 97486_Patient-09sk_skeletal muscle | 0.0 |
| 97487_Patient-09ut_uterus | 0.0 |
| 97488_Patient-09pl_placenta | 0.0 |
| 97492_Patient-10ut_uterus | 0.0 |
| 97493_Patient-l0pl_placenta | 0.0 |
| 97495_Patient-11go_adipose | 1.0 |
| 97496_Patient-11sk_skeletal muscle | 0.0 |
| 97497_Patient-11ut_uterus | 0.0 |
| 97498_Patient-11pl_placenta | 0.0 |
| 97500_Patient-12go_adipose | 2.1 |
| 97501_Patient-12sk_skeletal muscle | 0.0 |
| 97502_Patient-12ut_uterus | 0.0 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 30.1 |
| 73556_Heart_Cardiac stromal cells (primary) | 0.4 |
| 81735_Small Intestine | 17.9 |
| 72409_Kidney_Proximal Convoluted Tubule | 1.6 |
| 82685_Small intestine_Duodenum | 100.0 |
| 90650_Adrenal_Adrenocortical adenoma | 0.0 |
| 72410_Kidney_HRCE | 9.3 |
| 72411_Kidney_HRE | 0.0 |
| 73139_Uterus_Uterine smooth muscle cells | 0.7 |

Column A - Rel. Exp. (%) Ag1681, Run 248029382

TABLE HG
Panel 5D

| Tissue Name | A |
|---|---|
| 97457_Patient-02go_adipose | 0.2 |
| 97476_Patient-07sk_skeletal muscle | 0.0 |
| 97477_Patient-07ut_uterus | 0.0 |
| 97478_Patient-07pl_placenta | 0.0 |
| 9748l_Patient-08sk_skeletal muscle | 0.0 |
| 97482_Patient-08ut_uterus | 0.0 |
| 97483_Patient-08pl_placenta | 0.2 |
| 97486_Patient-09sk_skeletal muscle | 0.0 |
| 97487_Patient-09ut_uterus | 0.1 |
| 97488_Patient-09pl_placenta | 0.0 |
| 97492_Patient-1Out_uterus | 0.0 |
| 97493_Patient-1Opl_placenta | 0.0 |
| 97495_Patient-11go_adipose | 0.0 |
| 97496_Patient-11sk_skeletal muscle | 0.0 |
| 97497_Patient-11ut_uterus | 0.0 |
| 97498_Patient-11pl_placenta | 0.0 |
| 97500_Patient-12go_adipose | 0.3 |
| 97501_Patient-12sk_skeletal muscle | 0.0 |
| 97502_Patient-12ut_uterus | 0.2 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 28.3 |
| 73556_Heart_Cardiac stromal cells (primary) | 0.0 |
| 81735_Small Intestine | 12.3 |
| 72409_Kidney_Proximal Convoluted Tubule | 4.3 |
| 82685_Small intestine_Duodenum | 100.0 |
| 90650_Adrenal_Adrenocortical adenoma | 0.0 |
| 72410_Kidney_HRCE | 3.6 |
| 72411_Kidney_HRE | 0.1 |
| 73139_Uterus_Uterine smooth muscle cells | 0.0 |

Column A - Rel. Exp. (%) Ag1681, Run 169271477

General_screening_panel_v1.4 Summary: Ag1681 The CG56151-01 gene, a glucose transporter type 2 homolog, is predominantly expressed in liver. GLUT2 facilitates the transport of glucose into the liver. This gene is also expressed in brain, pancreas, and testis. This is consistent with immunocytochemistry data that shows that the Glut2 gene is expressed in insulin producing beta cells in the pancreas and aids in regulation of insulin secretion. Since the liver is responsible for gluconeogenesis, enhancing glucose uptake through GLUT2 may produce a negative feedback loop that would decrease hepatic glucose production. This could result in a lowering of blood glucose, a major therapeutic goal for the treatment of Type II (non-insulin dependent) diabetes. Thus, enhancing the function of the protein encoded by the CG56151-01 gene with an agonist antibody therapeutic could restore balance to blood glucose levels in patients with Type II diabetes.

In addition, this gene is expressed at higher levels in fetal liver and lung (CTs=29) than in the adult sources of-these tissues. Thus, expression of this gene could be used to differentiate disorders or predisposition to disorders between the two sources of these tissues.

References:

Waeber G, et al. Mol Cell Endocrinol Oct. 30, 1995;114(1-2):205-15. (PMID: 8674846)

Human Metabolic Summary: Ag1681 Highest expression is seen in normal liver (CT=22)samples, with lower expression seen in liver samples from diabetic patients.

Panel 1.3D Summary: Ag1681 Expression of the CG56151-01 gene is restricted to liver derived tissue, an important metabolic tissue, in this panel (CTs=27). This liver specific expression is consistent with expression in other panels and with published data (see reference below.) Thus, expression of this gene could be used as a marker for liver tissue. This gene encodes a glut2 homolog. Please see General_screening_panel_v1.4 for disscussion of this gene in metabolic disease.

References:

Rencurel F, et al. Biochem J Mar. 15, 1996;314 (Pt 3):903-9. (PMID: 8615787)

Panel 2D Summary: Ag1681 The expression of the CG56151-01 gene appears to be highest in a sample of normal liver tissue adjacent to a colon cancer metastasis (CT=24.6). In addition, there is substantial expression in both normal and malignant liver tissue. This restricted pattern of expression in liver derived tissue is consistent with expression in the previous panels. Moreover, therapeutic modulation of this gene, through the use of small molecule drugs, protein therapeutics or antibodies might be benefical in the treatment of liver cancer.

Panel 5 Islet Summary: Ag1681 Moderate expression is seen in samples derived from small intestine and a liver cell line.

Panel 5D Summary: Ag1681 The expression pattern of the CG56151-01 gene, a Glut2 homolog, is limited to a liver cell line (HepG2) and small intestines. The presence of this isoform in the intestines may indicate an important role in glucose uptake from the digestive tract. Please refer to panel 1.4 for a further discussion of this gene in metabolic disease.

I. CG56155-02: Plasma Kallikrein Precursor

Expression of gene CG56155-02 was assessed using the primer-probe set Ag1688, described in Table IA. Results of the RTQ-PCR runs are shown in Tables IB, IC, ID, IE, IF, IG and IH.

TABLE IA
Probe Name Ag1688

| | | | Start | SEQ ID |
|---|---|---|---|---|
| Primers | Sequences | Length | Position | No |
| Forward | 5′-tcagaagggaatcatgatatcg-3′ | 22 | 577 | 122 |
| Probe | TET-5′-ccttgataaaactccaggctcctttga-3′-TAMRA | 27 | 550 | 123 |
| Reverse | 5′-tttggaaggtaggcatattgg-3′ | 21 | 509 | 124 |

TABLE IB
AI_comprehensive panel_v1.0

| Tissue Name | A |
|---|---|
| 110967 COPD-F | 53.6 |
| 110980 COPD-F | 14.2 |
| 110968 COPD-M | 48.3 |
| 110977 COPD-M | 53.6 |
| 110989 Emphysema-F | 61.6 |
| 110992 Emphysema-F | 21.6 |
| 110993 Emphysema-F | 23.8 |
| 110994 Emphysema-F | 20.7 |
| 110995 Emphysema-F | 55.1 |
| 110996 Emphysema-F | 17.8 |
| 110997 Asthma-M | 25.2 |
| 111001 Asthma-F | 23.0 |
| 111002 Asthma-F | 22.1 |
| 111003 Atopic Asthma-F | 15.5 |
| 111004 Atopic Asthma-F | 19.9 |
| 111005 Atopic Asthma-F | 23.8 |
| 111006 Atopic Asthma-F | 6.0 |
| 111417 Allergy-M | 4.6 |
| 112347 Allergy-M | 0.0 |
| 112349 Normal Lung-F | 0.0 |
| 112357 Normal Lung-F | 38.7 |
| 112354 Normal Lung-M | 24.0 |
| 112374 Crohns-F | 10.4 |
| 112389 Match Control Crohns-F | 7.4 |
| 112375 Crohns-F | 4.6 |
| 112732 Match Control Crohns-F | 25.0 |
| 112725 Crohns-M | 11.3 |
| 112387 Match Control Crohns-M | 1.0 |
| 112378 Crohns-M | 0.0 |
| 112390 Match Control Crohns-M | 44.1 |
| 112726 Crohns-M | 19.5 |
| 112731 Match Control Crohns-M | 58.2 |
| 112380 Ulcer Col-F | 3.2 |
| 112734 Match Control Ulcer Col-F | 56.6 |
| 112384 Ulcer Col-F | 10.1 |
| 112737 Match Control Ulcer Col-F | 21.6 |
| 112386 Ulcer Col-F | 0.0 |
| 112738 Match Control Ulcer Col-F | 9.3 |
| 112381 Ulcer Col-M | 0.0 |
| 112735 Match Control Ulcer Col-M | 41.8 |
| 112382 Ulcer Col-M | 3.8 |
| 112394 Match Control Ulcer Col-M | 5.2 |
| 112383 Ulcer Col-M | 31.6 |
| 112736 Match Control Ulcer Col-M | 12.9 |
| 112423 Psoriasis-F | 9.2 |
| 112427 Match Control Psoriasis-F | 77.4 |
| 112418 Psoriasis-M | 12.7 |
| 112723 Match Control Psoriasis-M | 0.0 |
| 112419 Psoriasis-M | 100.0 |
| 112424 Match Control Psoriasis-M | 35.6 |
| 112420 Psoriasis-M | 87.7 |
| 112425 Match Control Psoriasis-M | 29.1 |
| 104689 (MF) OA Bone-Backus | 50.0 |
| 104690 (MF) Adj “Normal” Bone-Backus | 34.9 |
| 104691 (MF) OA Synovium-Backus | 25.5 |
| 104692 (BA) OA Cartilage-Backus | 37.6 |
| 104694 (BA) OA Bone-Backus | 8.4 |
| 104695 (BA) Adj “Normal” Bone-Backus | 34.4 |
| 104696 (BA) OA Synovium-Backus | 6.9 |
| 104700 (SS) OA Bone-Backus | 22.8 |
| 104701 (SS) Adj “Normal” Bone-Backus | 42.3 |
| 104702 (SS) OA Synovium-Backus | 29.5 |
| 117093 OA Cartilage Rep7 | 10.6 |
| 112672 OA Bone5 | 94.0 |
| 112673 OA Synovium5 | 43.2 |
| 112674 OA Synovial Fluid cells5 | 58.6 |
| 117100 OA Cartilage Rep 14 | 0.0 |
| 112756 OA Bone9 | 2.6 |
| 112757 OA Synovium9 | 8.0 |
| 112758 OA Synovial Fluid Cells9 | 22.1 |
| 117125 RA Cartilage Rep2 | 22.1 |
| 113492 Bone2 RA | 10.0 |
| 113493 Synovium2 RA | 11.0 |
| 113494 Syn Fluid Cells RA | 31.6 |
| 113499 Cartilage4 RA | 47.6 |
| 113500 Bone4 RA | 37.9 |
| 113501 Synovium4 RA | 55.5 |
| 113502 Syn Fluid Cells4 RA | 10.0 |
| 113495 Cartilage3 RA | 20.7 |
| 113496 Bone3 RA | 16.2 |
| 113497 Synovium3 RA | 11.5 |
| 113498 Syn Fluid Cells3 RA | 25.3 |
| 117106 Normal Cartilage Rep20 | 0.0 |
| 113663 Bone3 Normal | 0.9 |
| 113664 Synovium3 Normal | 0.0 |
| 113665 Syn Fluid Cells3 Normal | 1.1 |
| 117107 Normal Cartilage Rep22 | 2.7 |
| 113667 Bone4 Normal | 8.1 |
| 113668 Synovium4 Normal | 5.8 |
| 113669 Syn Fluid Cells4 Normal | 5.3 |

Column A - Rel. Exp. (%) Ag1688, Run 248429492

TABLE IC
CNS_neurodegeneration_v1.0

| Tissue Name | A |
|---|---|
| AD 1 Hippo | 24.5 |
| AD 2 Hippo | 34.4 |
| AD 3 Hippo | 17.9 |
| AD 4 Hippo | 18.0 |
| AD 5 hippo | 94.6 |
| AD 6 Hippo | 34.9 |
| Control 2 Hippo | 35.4 |
| Control 4 Hippo | 50.7 |
| Control (Path) 3 Hippo | 9.3 |
| AD 1 Temporal Ctx | 31.9 |
| AD 2 Temporal Ctx | 31.4 |
| AD 3 Temporal Ctx | 20.4 |
| AD 4 Temporal Ctx | 29.5 |
| AD 5 Inf Temporal Ctx | 100.0 |
| AD 5 Sup Temporal Ctx | 92.0 |
| AD 6 Inf Temporal Ctx | 43.8 |
| AD 6 Sup Temporal Ctx | 69.7 |
| Control 1 Temporal Ctx | 16.5 |
| Control 2 Temporal Ctx | 34.9 |
| Control 3 Temporal Ctx | 32.3 |
| Control 4 Temporal Ctx | 35.4 |
| Control (Path) 1 Temporal Ctx | 46.0 |
| Control (Path) 2 Temporal Ctx | 45.7 |
| Control (Path) 3 Temporal Ctx | 9.7 |
| Control (Path) 4 Temporal Ctx | 41.8 |
| AD 1 Occipital Ctx | 42.3 |
| AD 2 Occipital Ctx (Missing) | 0.0 |
| AD 3 Occipital Ctx | 7.1 |
| AD 4 Occipital Ctx | 26.4 |
| AD 5 Occipital Ctx | 9.9 |
| AD 6 Occipital Ctx | 27.2 |
| Control 1 Occipital Ctx | 6.3 |
| Control 2 Occipital Ctx | 49.7 |
| Control 3 Occipital Ctx | 39.2 |
| Control 4 Occipital Ctx | 26.6 |
| Control (Path) 1 Occipital Ctx | 47.3 |
| Control (Path) 2 Occipital Ctx | 21.3 |
| Control (Path) 3 Occipital Ctx | 3.5 |
| Control (Path) 4 Occipital Ctx | 17.8 |
| Control 1 Parietal Ctx | 19.5 |
| Control 2 Parietal Ctx | 85.3 |
| Control 3 Parietal Ctx | 15.5 |
| Control (Path) 1 Parietal Ctx | 44.4 |
| Control (Path) 2 Parietal Ctx | 52.9 |
| Control (Path) 3 Parietal Ctx | 9.7 |
| Control (Path) 4 Parietal Ctx | 52 1 |

Column A - Rel. Exp. (%) Ag1688, Run 269217573

TABLE ID
Panel 1.3D

| Tissue Name | A |
|---|---|
| Liver adenocarcinoma | 0.0 |
| Pancreas | 6.7 |
| Pancreatic ca. CAPAN 2 | 0.2 |
| Adrenal gland | 1.8 |
| Thyroid | 3.8 |
| Salivary gland | 1.5 |
| Pituitary gland | 6.1 |
| Brain (fetal) | 0.5 |
| Brain (whole) | 3.6 |
| Brain (amygdala) | 3.3 |
| Brain (cerebellum) | 0.4 |
| Brain (hippocampus) | 6.2 |
| Brain (substantia nigra) | 1.0 |
| Brain (thalamus) | 2.1 |
| Cerebral Cortex | 6.3 |
| Spinal cord | 3.1 |
| glio/astro U87-MG | 0.0 |
| glio/astro U-118-MG | 0.0 |
| astrocytoma SW1783 | 0.0 |
| neuro*; met SK-N-AS | 0.2 |
| astrocytoma SF-539 | 0.0 |
| astrocytoma SNB-75 | 0.1 |
| glioma SNB-19 | 0.2 |
| glioma U251 | 1.2 |
| glioma SF-295 | 0.0 |
| Heart (fetal) | 0.2 |
| Heart | 1.6 |
| Skeletal muscle (fetal) | 0.7 |
| Skeletal muscle | 1.2 |
| Bone marrow | 0.5 |
| Thymus | 3.2 |
| Spleen | 1.0 |
| Lymph node | 2.9 |
| Colorectal | 0.8 |
| Stomach | 3.3 |
| Small intestine | 6.2 |
| Colon ca. SW480 | 0.0 |
| Colon ca.* SW620(SW480 met) | 0.0 |
| Colon ca. HT29 | 0.0 |
| Colon ca. HCT-116 | 0.0 |
| Colon ca. CaCo-2 | 0.2 |
| Colon ca. tissue(ODO3866) | 0.0 |
| Colon ca. HCC-2998 | 0.2 |
| Gastric ca.* (liver met) NCI-N87 | 4.4 |
| Bladder | 3.1 |
| Trachea | 3.0 |
| Kidney | 6.8 |
| Kidney (fetal) | 9.2 |
| Renal ca. 786-0 | 0.0 |
| Renal ca. A498 | 1.7 |
| Renal ca. RXF 393 | 0.0 |
| Renal ca. ACHN | 0.0 |
| Renal ca. UO-31 | 0.0 |
| Renal ca. TK-10 | 0.0 |
| Liver | 100.0 |
| Liver (fetal) | 99.3 |
| Liver ca. (hepatoblast) HepG2 | 0.0 |
| Lung | 1.3 |
| Lung (fetal) | 1.8 |
| Lung ca. (small cell) LX-1 | 0.0 |
| Lung ca. (small cell) NCI-H69 | 0.0 |
| Lung ca. (s. cell var.) SHP-77 | 0.8 |
| Lung ca. (large cell)NCI-H460 | 0.0 |
| Lung ca. (non-sm. cell) A549 | 0.2 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.2 |
| Lung ca. (squam.) NCI-H596 | 0.0 |
| Mammary gland | 2.9 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.2 |
| Ovarian ca. OVCAR-4 | 0.0 |
| Ovarian ca. OVCAR-5 | 0.3 |
| Ovarian ca. OVCAR-8 | 0.0 |
| Ovarian ca. IGROV-1 | 0.0 |
| Ovarian ca.* (ascites) SK-OV-3 | 1.0 |
| Uterus | 1.4 |
| Placenta | 0.4 |
| Prostate | 1.0 |
| Prostate ca.* (bone met)PC-3 | 0.0 |
| Testis | 6.1 |
| Melanoma Hs688(A).T | 0.4 |
| Melanoma* (met) Hs688(B).T | 0.9 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 0.5 |

Column A - Rel. Exp. (%) Ag1688, Run 147249266

TABLE IE
Panel 2D

| Tissue Name | A |
|---|---|
| Normal Colon | 1.7 |
| CC Well to Mod Diff (ODO3866) | 0.0 |
| CC Margin (ODO3866) | 0.2 |
| CC Gr.2 rectosigmoid (ODO3868) | 0.2 |
| CC Margin (ODO3868) | 0.1 |
| CC Mod Diff (ODO3920) | 0.1 |
| CC Margin (ODO3920) | 0.9 |
| CC Gr.2 ascend colon (ODO3921) | 0.1 |
| CC Margin (ODO3921) | 0.1 |
| CC from Partial Hepatectomy (ODO4309) Mets | 4.7 |
| Liver Margin (ODO4309) | 100.0 |
| Colon mets to lung (OD04451-01) | 0.1 |
| Lung Margin (OD04451-02) | 0.1 |
| Normal Prostate 6546-1 | 2.1 |
| Prostate Cancer (OD04410) | 0.6 |
| Prostate Margin (OD04410) | 0.5 |
| Prostate Cancer (OD04720-01) | 1.1 |
| Prostate Margin (OD04720-02) | 1.6 |
| Normal Lung 061010 | 2.0 |
| Lung Met to Muscle (ODO4286) | 0.0 |
| Muscle Margin (ODO4286) | 0.2 |
| Lung Malignant Cancer (OD03126) | 0.1 |
| Lung Margin (OD03126) | 0.5 |
| Lung Cancer (OD04404) | 0.1 |
| Lung Margin (OD04404) | 0.2 |
| Lung Cancer (OD04565) | 0.0 |
| Lung Margin (OD04565) | 0.1 |
| Lung Cancer (OD04237-01) | 0.1 |
| Lung Margin (OD04237-02) | 0.4 |
| Ocular Mel Met to Liver (ODO4310) | 0.1 |
| Liver Margin (ODO4310) | 77.4 |
| Melanoma Mets to Lung (OD04321) | 0.0 |
| Lung Margin (OD04321) | 0.1 |
| Normal Kidney | 12.9 |
| Kidney Ca, Nuclear grade 2 (OD04338) | 3.8 |
| Kidney Margin (OD04338) | 1.6 |
| Kidney Ca Nuclear grade 1/2 (OD04339) | 2.8 |
| Kidney Margin (OD04339) | 9.3 |
| Kidney Ca, Clear cell type (OD04340) | 1.4 |
| Kidney Margin (OD04340) | 4.1 |
| Kidney Ca, Nuclear grade 3 (OD04348) | 0.1 |
| Kidney Margin (OD04348) | 3.8 |
| Kidney Cancer (OD04622-01) | 0.2 |
| Kidney Margin (OD04622-03) | 0.7 |
| Kidney Cancer (OD04450-01) | 0.2 |
| Kidney Margin (OD04450-03) | 2.6 |
| Kidney Cancer 8120607 | 0.0 |
| Kidney Margin 8120608 | 0.7 |
| Kidney Cancer 8120613 | 0.0 |
| Kidney Margin 8120614 | 0.5 |
| Kidney Cancer 9010320 | 0.2 |
| Kidney Margin 9010321 | 1.0 |
| Normal Uterus | 0.2 |
| Uterus Cancer 064011 | 0.8 |
| Normal Thyroid | 0.9 |
| Thyroid Cancer 064010 | 0.2 |
| Thyroid Cancer A302152 | 0.5 |
| Thyroid Margin A302153 | 1.0 |
| Normal Breast | 0.3 |
| Breast Cancer (OD04566) | 0.1 |
| Breast Cancer (OD04590-01) | 0.1 |
| Breast Cancer Mets (OD04590-03) | 0.4 |
| Breast Cancer Metastasis (OD04655-05) | 0.9 |
| Breast Cancer 064006 | 0.6 |
| Breast Cancer 1024 | 1.2 |
| Breast Cancer 9100266 | 0.1 |
| Breast Margin 9100265 | 0.1 |
| Breast Cancer A209073 | 0.3 |
| Breast Margin A209073 | 0.3 |
| Normal Liver | 69.7 |
| Liver Cancer 064003 | 13.7 |
| Liver Cancer 1025 | 18.0 |
| Liver Cancer 1026 | 1.2 |
| Liver Cancer 6004-T | 22.2 |
| Liver Tissue 6004-N | 1.0 |
| Liver Cancer 6005-T | 1.9 |
| Liver Tissue 6005-N | 4.2 |
| Normal Bladder | 2.7 |
| Bladder Cancer 1023 | 0.0 |
| Bladder Cancer A302173 | 0.2 |
| Bladder Cancer (OD04718-01) | 0.1 |
| Bladder Normal Adjacent (OD04718-03) | 0.5 |
| Normal Ovary | 0.0 |
| Ovarian Cancer 064008 | 0.1 |
| Ovarian Cancer (OD04768-07) | 0.2 |
| Ovary Margin (OD04768-08) | 0.1 |
| Normal Stomach | 0.3 |
| Gastric Cancer 9060358 | 0.1 |
| Stomach Margin 9060359 | 0.0 |
| Gastric Cancer 9060395 | 0.2 |
| Stomach Margin 9060394 | 0.3 |
| Gastric Cancer 9060397 | 0.3 |
| Stomach Margin 9060396 | 0.0 |
| Gastric Cancer 064005 | 1.1 |

Column A - Rel. Exp. (%) Ag1688, Run 162646059

TABLE IF
Panel 4.1D

| Tissue Name | A |
|---|---|
| Secondary Th1 act | 1.6 |
| Secondary Th2 act | 1.7 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 1.3 |
| Primary Th2 rest | 1.3 |
| Primary Tr1 rest | 1.6 |
| CD45RA CD4 lymphocyte act | 3.5 |
| CD45RO CD4 lymphocyte act | 4.2 |
| CD8 lymphocyte act | 3.2 |
| Secondary CD8 lymphocyte rest | 1.8 |
| Secondary CD8 lymphocyte act | 0.0 |
| CD4 lymphocyte none | 3.8 |
| 2ry Th1/Th2/Tr1_anti-CD95 CH11 | 0.0 |
| LAK cells rest | 0.0 |
| LAK cells IL-2 | 6.2 |
| LAK cells IL-2 + IL-12 | 0.0 |
| LAK cells IL-2 + IFN gamma | 1.7 |
| LAK cells IL-2 + IL-18 | 3.4 |
| LAK cells PMA/ionomycin | 0.0 |
| NK Cells IL-2 rest | 22.1 |
| Two Way MLR 3 day | 3.3 |
| Two Way MLR 5 day | 1.9 |
| Two Way MLR 7 day | 1.7 |
| PBMC rest | 1.5 |
| PBMC PWM | 5.1 |
| PBMC PHA-L | 0.7 |
| Ramos (B cell) none | 0.0 |
| Ramos (B cell) ionomycin | 0.0 |
| B lymphocytes PWM | 2.8 |
| B lymphocytes CD40L and IL-4 | 21.5 |
| EOL-1 dbcAMP | 0.0 |
| EOL-1 dbcAMP PMA/ionomycin | 0.0 |
| Dendritic cells none | 2.0 |
| Dendritic cells LPS | 0.0 |
| Dendritic cells anti-CD40 | 4.9 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 2.4 |
| KU-812 (Basophil) rest | 1.8 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 100.0 |
| NCI-H292 none | 0.0 |
| NCI-H292 IL-4 | 1.5 |
| NCI-H292 IL-9 | 1.9 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 2.6 |
| Lung fibroblast TNF alpha + IL-1 beta | 10.4 |
| Lung fibroblast IL-4 | 1.8 |
| Lung fibroblast IL-9 | 12.3 |
| Lung fibroblast IL-13 | 0.0 |
| Lung fibroblast IFN gamma | 3.1 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 6.8 |
| Dermal fibroblast IL-4 | 5.8 |
| Dermal Fibroblasts rest | 0.0 |
| Neutrophils TNFa + LPS | 0.0 |
| Neutrophils rest | 0.0 |
| Colon | 0.0 |
| Lung | 1.2 |
| Thymus | 0.0 |
| Kidney | 82.9 |

Column A - Rel. Exp. (%) Ag1688, Run 248389308

TABLE IG
Panel 5 Islet

| Tissue Name | A |
|---|---|
| 97457_Patient-02go_adipose | 41.2 |
| 97476_Patient-07sk_skeletal muscle | 9.9 |
| 97477_Patient-07ut_uterus | 8.1 |
| 97478_Patient-07pl_placenta | 0.0 |
| 99167_Bayer Patient 1 | 84.7 |
| 97482_Patient-08ut_uterus | 2.4 |
| 97483_Patient-08pl_placenta | 0.0 |
| 97486_Patient-09sk_skeletal muscle | 8.0 |
| 97487_Patient-09ut_uterus | 9.6 |
| 97488_Patient-09pl_placenta | 0.0 |
| 97492_Patient-10ut_uterus | 0.0 |
| 97493_Patient-10pl_placenta | 0.0 |
| 97495_Patient-11go_adipose | 0.0 |
| 97496_Patient-11sk_skeletal muscle | 52.9 |
| 97497_Patient-11ut_uterus | 35.8 |
| 97498_Patient-11pl_placenta | 10.5 |
| 97500_Patient-12go_adipose | 0.0 |
| 97501_Patient-12sk_skeletal muscle | 35.4 |
| 97502_Patient-12ut_uterus | 20.7 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 11.4 |
| 94713_Donor 2 AD - B_adipose | 0.0 |
| 94714_Donor 2 AD - C_adipose | 29.1 |
| 94742_Donor 3 U - A_Mesenchymal Stem Cells | 19.2 |
| 94743_Donor 3 U - B_Mesenchymal Stem Cells | 0.0 |
| 94730_Donor 3 AM - A_adipose | 15.0 |
| 94731_Donor 3 AM - B_adipose | 37.9 |
| 94732_Donor 3 AM - C_adipose | 0.0 |
| 94733_Donor 3 AD - A_adipose | 39.2 |
| 94734_Donor 3 AD - B_adipose | 11.4 |
| 94735_Donor 3 AD - C_adipose | 34.4 |
| 77138_Liver_HepG2untreated | 8.4 |
| 73556_Heart_Cardiac stromal cells (primary) | 0.0 |
| 81735_Small Intestine | 100.0 |
| 72409_Kidney_Proximal Convoluted Tubule | 9.9 |
| 82685_Small intestine_Duodenum | 70.2 |
| 90650_Adrenal_Adrenocortical adenoma | 25.5 |
| 72410_Kidney_HRCE | 10.4 |
| 72411_Kidney_HRE | 7.2 |
| 73139_Uterus_Uterine smooth muscle cells | 0.0 |

Column A - Rel. Exp. (%) Ag1688, Run 226587524

TABLE IH
general oncology screening panel_v_2.4

| Tissue Name | A |
|---|---|
| Colon cancer 1 | 1.8 |
| Colon cancer NAT 1 | 1.0 |
| Colon cancer 2 | 0.4 |
| Colon cancer NAT 2 | 1.2 |
| Colon cancer 3 | 0.8 |
| Colon cancer NAT 3 | 2.5 |
| Colon malignant cancer 4 | 2.1 |
| Colon normal adjacent tissue 4 | 0.2 |
| Lung cancer 1 | 0.2 |
| Lung NAT 1 | 0.2 |
| Lung cancer 2 | 1.0 |
| Lung NAT 2 | 0.8 |
| Squamous cell carcinoma 3 | 0.5 |
| Lung NAT 3 | 0.0 |
| metastatic melanoma 1 | 1.1 |
| Melanoma 2 | 0.1 |
| Melanoma 3 | 0.0 |
| metastatic melanoma 4 | 2.0 |
| metastatic melanoma 5 | 3.0 |
| Bladder cancer 1 | 0.6 |
| Bladder cancer NAT 1 | 0.0 |
| Bladder cancer 2 | 0.3 |
| Bladder cancer NAT 2 | 0.1 |
| Bladder cancer NAT 3 | 0.0 |
| Bladder cancer NAT 4 | 1.1 |
| Prostate adenocarcinoma 1 | 3.7 |
| Prostate adenocarcinoma 2 | 0.2 |
| Prostate adenocarcinoma 3 | 1.2 |
| Prostate adenocarcinoma 4 | 3.5 |
| Prostate cancer NAT 5 | 0.6 |
| Prostate adenocarcinoma 6 | 0.2 |
| Prostate adenocarcinoma 7 | 0.0 |
| Prostate adenocarcinoma 8 | 0.0 |
| Prostate adenocarcinoma 9 | 0.0 |
| Prostate cancer NAT 10 | 0.1 |
| Kidney cancer 1 | 7.7 |
| Kidney NAT 1 | 5.7 |
| Kidney cancer 2 | 40.1 |
| Kidney NAT 2 | 23.8 |
| Kidney cancer 3 | 100.0 |
| Kidney NAT 3 | 5.6 |
| Kidney cancer 4 | 2.0 |
| Kidney NAT 4 | 4.2 |

Column A - Rel. Exp. (%) Ag1688, Run 260552690

AI_comprehensive panel_v1.0 Summary: Ag1688 Highest expression of this gene is detected in psoriasis sample (CT=31.9). Moderate to low levels of expression of this gene is also seen in samples derived from ostcooarthitis/rheumatoid arthritis bone, cartilage, synovium and synovial fluid samples, from normal lung, COPD lung, emphysema, atopic asthma, asthma, Crohn's disease (normal matched control and diseased), ulcerative colitis(normal matched control and diseased), and psoriasis (normal matched control and diseased). Therefore, therapeutic modulation of this gene product may ameliorate symptoms/conditions associated with autoimmune and inflammatory disorders including psoriasis, asthma, inflammatory bowel disease, rheumatoid arthritis and osteoarthritis.

CNS_neurodegeneration_v1.0 Summary: Data obtained from RTQPCR assays performed for this panel was analyized using multivariate analysis (ANOVA). The multivariate results obtained analyzing Ag1688 confirms the expression of this gene at low levels in the brains of an independent group of individuals. However, no differential expression of this gene was detected between Alzheimer's diseased postmortem brains and those of non-demented controls in this experiment. Please see Panel 1.3D for a discussion of the potential role of this gene in treatment of central nervous system disorders.

Panel 1.3D Summary: Ag1688 Expression of this gene, a plasma kallikrein, is significantly higher in liver (CTs=28) than in any other sample on this panel. Thus, expression of this gene could be used as a marker of liver tissue. In addition, low levels of expression of this gene is also detected in tissues with metabolic/endocrine functions including pancreas, adrenal gland, thyroid, pituitary gland, skeletal muscle, heart, and the gastrointestinal tract. Plasma prekallikrein is a glycoprotein that participates in the surface-dependent activation of blood coagulation, fibrinolysis, kinin generation and inflammation. It is synthesized in the liver and secreted into the blood as a single polypeptide chain. It is converted to plasma kallikrein by factor XIIa. Recently, plasma kallikrein has been implicated in adipose differentiation by remodeling of the fibronectin-rich ECM of preadipocytes. Pig −/− mice show a reduction of fat deposit (Ref. 1, 2). At Curagen, it was found that plasma kallikrein significantly down-regulated in the liver of mice with ‘lean’ phenotype. Thus, based on Curagen GeneCalling data it is hypothesized that plasma kallikrein might cause disruption of adipose differentiation thus leading to obesity if over expressed and to a leaner phenotype if expression is below normal. Therefore, an antagonist to this gene product may be beneficial in the treatment of obesity.

Moderate to low levels of expression of this gene is also seen in some of the regions of central nervous system examined, including amygdala, hippocampus, substantia nigra, thalamus, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

References:

Hoover-Plow J, et al. Biochem.Biophys.Res.Commun. (2001) 284, 389-394. (PMID: 11394891).

Selvarajan S, et al. Nature Cell Biol. (2001) 3, 267-275. (PMID: 11231576)

Panel 2D Summary: Ag1688 The expression of the CG56155-01 gene appears to be highest in a sample derived from a sample of normal liver tissue adjacent to a metastatic colon cancer CT=26.2). In addition, there is substantial expression in other samples of normal liver, and to a much lesser degree, malignant liver tissue. This liver specific expression is consistent with the expression seen in Panel 1.3D.

Panel 4.1D Summary: Ag1688 Highest expression of this gene is detected in liver cirrhosis (CT=31.8). In addition, moderate to low levels of expression of this gene in IL-2 treated NK cells, CD40L and IL-4 treated B lymphocytes and normal kidney. Therefore, therapeutic modulation of the protein encoded for by this gene may be useful in the treatment of inflammatory or autoimmune diseases, liver cirrhosis and fibrosis, lupus erythematosus and glomerulonephritis.

Panel 5 Islet Summary: Ag1688 Expression of the CG56155-01 gene is limited to pancreatic islets and small intestines. Please see Panel 1.3 for discussion of this gene in metabolic disease.

general oncology screening panel_v_{—}2.4 Summary: Ag1688 Highest expression of this gene is detected in kidney cancer (CT=28.4). Higher expression of this gene is associated with cancer compared to normal kidney. Therefore, expression of this gene may be used as diagnostic marker for kidney cancer and therapeutic modulation of this gene or protein encoded by this gene may through the use of antibodies or small molecule drug may be useful in the treatment of kidney cancer.

J. CG56262-01: Ca-binding Transporter

Expression of gene CG56262-01 was assessed using the primer-probe sets Ag2896 and Ag2920, described in Tables JA and JB. Results of the RTQ-PCR runs are shown in Tables JC, JD, JE, JF and JG.

TABLE JA
Probe Name Ag2896

| | | | Start | SEQ ID |
|---|---|---|---|---|
| Primers | Sequences | Length | Position | No |
| Forward | 5′-gtcagcttctcttgctttgaga-3′ | 22 | 900 | 125 |
| Probe | TET-5′-cactgtcaggcactcgccaatgt-3′-TAMRA | 23 | 932 | 126 |
| Reverse | 5′-ctgtatttctggaagcattcca-3′ | 22 | 964 | 127 |

TABLE JB

Probe Name Ag2920

| | | | | |
|---|---|---|---|---|
| | | | Start | SEQ ID |
| Primers | Sequences | Length | Position | No |

| | | | | |
|---|---|---|---|---|
| Forward | 5′-ttgatgtctctgagatccaaca-3′ | 22 | 1134 | 128 |
| Probe | TET-5′-agtttccgagctctgggcatttccat-3′-TAMRA | 26 | 1107 | 129 |
| Reverse | 5′-catgctgtgcaaaattttctc-3′ | 21 | 1070 | 130 |

TABLE JC
CNS_neurodegeneration_v1.0

| Tissue Name | A | B |
|---|---|---|
| AD 1 Hippo | 17.7 | 21.6 |
| AD 2 Hippo | 41.5 | 40.3 |
| AD 3 Hippo | 13.9 | 18.2 |
| AD 4 Hippo | 10.7 | 11.3 |
| AD 5 Hippo | 65.1 | 54.7 |
| AD 6 Hippo | 62.4 | 73.2 |
| Control 2 Hippo | 45.4 | 51.8 |
| Control 4 Hippo | 15.8 | 19.8 |
| Control (Path) 3 Hippo | 10.6 | 12.9 |
| AD 1 Temporal Ctx | 17.6 | 18.9 |
| AD 2 Temporal Ctx | 41.5 | 41.8 |
| AD 3 Temporal Ctx | 10.1 | 12.9 |
| AD 4 Temporal Ctx | 29.9 | 27.5 |
| AD 5 Inf Temporal Ctx | 78.5 | 79.6 |
| AD 5 Sup Temporal Ctx | 47.0 | 43.5 |
| AD 6 Inf Temporal Ctx | 48.0 | 47.6 |
| AD 6 Sup Temporal Ctx | 47.3 | 55.5 |
| Control 1 Temporal Ctx | 14.8 | 16.6 |
| Control 2 Temporal Ctx | 53.6 | 66.9 |
| Control 3 Temporal Ctx | 24.3 | 22.8 |
| Control 3 Temporal Ctx | 18.0 | 15.8 |
| Control (Path) 1 Temporal Ctx | 86.5 | 88.3 |
| Control (Path) 2 Temporal Ctx | 45.1 | 50.0 |
| Control (Path) 3 Temporal Ctx | 11.0 | 15.3 |
| Control (Path) 4 Temporal Ctx | 41.2 | 36.9 |
| AD 1 Occipital Ctx | 10.7 | 13.1 |
| AD 2 Occipital Ctx (Missing) | 0.0 | 0.0 |
| AD 3 Occipital Ctx | 8.1 | 9.2 |
| AD 4 Occipital Ctx | 23.5 | 25.3 |
| AD 5 Occipital Ctx | 45.4 | 15.7 |
| AD 6 Occipital Ctx | 14.7 | 44.4 |
| Control 1 Occipital Ctx | 6.8 | 8.4 |
| Control 2 Occipital Ctx | 52.1 | 57.4 |
| Control 3 Occipital Ctx | 14.3 | 18.8 |
| Control 4 Occipital Ctx | 11.0 | 12.3 |
| Control (Path) 1 Occipital Ctx | 80.7 | 100.0 |
| Control (Path) 2 Occipital Ctx | 11.1 | 11.5 |
| Control (Path) 3 Occipital Ctx | 5.3 | 7.0 |
| Control (Path) 4 Occipital Ctx | 14.2 | 12.3 |
| Control 1 Parietal Ctx | 13.8 | 15.4 |
| Control 2 Parietal Ctx | 40.6 | 40.6 |
| Control 3 Parietal Ctx | 2.8 | 17.8 |
| Control (Path) 1 Parietal Ctx | 100.0 | 100.0 |
| Control (Path) 2 Parietal Ctx | 25.0 | 23.2 |
| Control (Path) 3 Parietal Ctx | 7.1 | 9.7 |
| Control (Path) 4 Parietal Ctx | 46.3 | 47.6 |

Column A - Rel. Exp. (%) Ag2896, Run 209734744
Column B - Rel. Exp. (%) Ag2920, Run 209779301

TABLE JD
Panel 1.3D

| Tissue Name | A | B |
|---|---|---|
| Liver adenocarcinoma | 36.6 | 40.1 |
| Pancreas | 4.2 | 7.4 |
| Pancreatic ca. CAPAN 2 | 10.1 | 9.3 |
| Adrenal gland | 3.3 | 2.8 |
| Thyroid | 11.8 | 18.9 |
| Salivary gland | 6.7 | 6.6 |
| Pituitary gland | 2.2 | 2.7 |
| Brain (fetal) | 27.0 | 27.7 |
| Brain (whole) | 81.2 | 74.2 |
| Brain (amygdala) | 40.1 | 40.3 |
| Brain (cerebellum) | 30.8 | 33.0 |
| Brain (hippocampus) | 44.8 | 42.0 |
| Brain (substantia nigra) | 23.0 | 21.5 |
| Brain (thalamus) | 25.5 | 31.6 |
| Cerebral Cortex | 100.0 | 100.0 |
| Spinal cord | 12.6 | 12.9 |
| glio/astro U87-MG | 2.2 | 2.4 |
| glio/astro U-118-MG | 9.9 | 8.1 |
| astrocytoma SW1783 | 8.8 | 10.1 |
| neuro*; met SK-N-AS | 4.2 | 3.3 |
| astrocytoma SF-539 | 5.8 | 5.4 |
| astrocytoma SNB-75 | 10.2 | 10.5 |
| glioma SNB-19 | 10.1 | 11.0 |
| glioma U251 | 14.1 | 15.8 |
| glioma SF-295 | 6.0 | 5.9 |
| Heart (fetal) | 38.7 | 40.1 |
| Heart | 10.7 | 9.9 |
| Skeletal muscle (fetal) | 16.0 | 11.8 |
| Skeletal muscle | 31.2 | 28.7 |
| Bone marrow | 0.4 | 0.6 |
| Thymus | 2.5 | 2.5 |
| Spleen | 1.1 | 1.4 |
| Lymph node | 2.6 | 1.7 |
| Colorectal | 14.0 | 12.3 |
| Stomach | 4.4 | 4.0 |
| Small intestine | 4.4 | 4.9 |
| Colon ca. SW480 | 5.1 | 5.3 |
| Colon ca.* SW620 (SW480 met) | 14.9 | 18.7 |
| Colon ca. HT29 | 6.8 | 7.2 |
| Colon ca. HCT-116 | 10.7 | 10.4 |
| Colon ca. CaCo-2 | 17.0 | 21.9 |
| Colon ca. tissue (ODO3866) | 2.4 | 2.4 |
| Colon ca. HCC-2998 | 9.3 | 7.6 |
| Gastric ca.* (liver met) NCI-N87 | 5.7 | 5.5 |
| Bladder | 5.1 | 5.1 |
| Trachea | 1.9 | 2.8 |
| Kidney | 12.4 | 17.0 |
| Kidney (fetal) | 23.2 | 21.6 |
| Renal ca. 786-0 | 15.5 | 19.6 |
| Renal ca. A498 | 9.5 | 9.4 |
| Renal ca. RXF 393 | 17.3 | 16.6 |
| Renal ca. ACHN | 10.5 | 14.5 |
| Renal ca. UO-31 | 7.7 | 9.9 |
| Renal ca. TK-10 | 12.4 | 14.7 |
| Liver | 4.3 | 3.5 |
| Liver (fetal) | 1.8 | 2.6 |
| Liver ca. (hepatoblast) HepG2 | 4.7 | 4.9 |
| Lung | 5.4 | 3.2 |
| Lung (fetal) | 4.8 | 4.7 |
| Lung ca. (small cell) LX-1 | 6.7 | 6.2 |
| Lung ca. (small cell) NCI-H69 | 0.0 | 0.1 |
| Lung ca. (s. cell var.) SHP-77 | 26.1 | 31.9 |
| Lung ca. (large cell) NCI-H460 | 1.2 | 1.4 |
| Lung ca. (non-sm. cell) A549 | 10.4 | 8.9 |
| Lung ca. (non-s. cell) NCI-H23 | 11.0 | 12.7 |
| Lung ca. (non-s. cell) HOP-62 | 4.9 | 4.7 |
| Lung ca. (non-s. cl) NCI-H522 | 11.4 | 11.4 |
| Lung ca. (squam.) SW 900 | 7.9 | 8.6 |
| Lung ca. (squam.) NCI-H596 | 0.3 | 0.4 |
| Mammary gland | 8.3 | 8.5 |
| Breast ca.* (pl. ef) MCF-7 | 7.5 | 8.1 |
| Breast ca.* (pl. ef) MDA-MB-231 | 6.6 | 7.1 |
| Breast ca.* (pl. ef) T47D | 16.2 | 17.0 |
| Breast ca. BT-549 | 5.8 | 5.1 |
| Breast ca. MDA-N | 19.5 | 22.5 |
| Ovary | 10.4 | 10.3 |
| Ovarian ca. OVCAR-3 | 11.5 | 9.2 |
| Ovarian ca. OVCAR-4 | 35.6 | 32.5 |
| Ovarian ca. OVCAR-5 | 31.0 | 34.2 |
| Ovarian ca. OVCAR-8 | 5.4 | 5.5 |
| Ovarian ca. IGROV-1 | 10.3 | 10.2 |
| Ovarian ca.* (ascites) SK-OV-3 | 13.8 | 18.0 |
| Uterus | 6.3 | 8.4 |
| Placenta | 0.0 | 0.0 |
| Prostate | 3.2 | 3.8 |
| Prostate ca.* (bone met) PC-3 | 16.5 | 18.3 |
| Testis | 1.4 | 1.3 |
| Melanoma Hs688(A).T | 2.8 | 2.6 |
| Melanoma* (met) Hs688(B).T | 2.6 | 3.8 |
| Melanoma UACC-62 | 10.9 | 11.2 |
| Melanoma M14 | 8.6 | 5.8 |
| Melanoma LOX IMVI | 12.2 | 10.8 |
| Melanoma* (met) SK-MEL-5 | 24.0 | 25.2 |
| Adipose | 6.1 | 6.4 |

Column A - Rel. Exp. (%) Ag2896, Run 167660338
Column B - Rel. Exp. (%) Ag2920, Run 167646813

TABLE JE
Panel 4D

| Tissue Name | A | B |
|---|---|---|
| Secondary Th1 act | 5.6 | 7.3 |
| Secondary Th2 act | 6.8 | 6.0 |
| Secondary Tr1 act | 7.4 | 7.7 |
| Secondary Th1 rest | 6.4 | 6.7 |
| Secondary Th2 rest | 7.6 | 7.1 |
| Secondary Tr1 rest | 12.2 | 9.7 |
| Primary Th1 act | 12.7 | 14.5 |
| Primary Th2 act | 16.0 | 15.1 |
| Primary Tr1 act | 26.4 | 22.1 |
| Primary Th1 rest | 31.6 | 33.4 |
| Primary Th2 rest | 19.3 | 18.7 |
| Primary Tr1 rest | 14.7 | 16.5 |
| CD45RA CD4 lymphocyte act | 6.2 | 5.1 |
| CD45RO CD4 lymphocyte act | 11.2 | 12.3 |
| CD8 lymphocyte act | 11.6 | 10.8 |
| Secondary CD8 lymphocyte rest | 8.2 | 9.9 |
| Secondary CD8 lymphocyte act | 3.0 | 3.3 |
| CD4 lymphocyte none | 2.8 | 3.4 |
| 2ry Th1/Th2/Tr1_anti-CD95 CH11 | 7.3 | 7.4 |
| LAK cells rest | 7.1 | 6.7 |
| LAK cells IL-2 | 14.7 | 17.0 |
| LAK cells IL-2 + IL-12 | 6.9 | 7.0 |
| LAK cells IL-2 + IFN gamma | 12.8 | 11.0 |
| LAK cells IL-2 + IL-18 | 6.7 | 9.0 |
| LAK cells PMA/ionomycin | 0.9 | 0.6 |
| NK Cells IL-2 rest | 7.2 | 6.5 |
| Two Way MLR 3 day | 6.3 | 6.8 |
| Two Way MLR 5 day | 3.5 | 3.0 |
| Two Way MLR 7 day | 4.2 | 4.5 |
| PBMC rest | 2.0 | 1.6 |
| PBMC PWM | 27.9 | 26.2 |
| PBMC PHA-L | 27.5 | 26.8 |
| Ramos (B cell) none | 16.8 | 16.0 |
| Ramos (B cell) ionomycin | 100.0 | 100.0 |
| B lymphocytes PWM | 36.6 | 22.8 |
| B lymphocytes CD40L and IL-4 | 13.5 | 14.9 |
| EOL-1 dbcAMP | 14.5 | 15.5 |
| EOL-1 dbcAMP PMA/ionomycin | 7.1 | 6.3 |
| Dendritic cells none | 0.8 | 1.5 |
| Dendritic cells LPS | 0.1 | 0.2 |
| Dendritic cells anti-CD40 | 0.9 | 0.7 |
| Monocytes rest | 0.1 | 0.0 |
| Monocytes LPS | 0.2 | 0.0 |
| Macrophages rest | 4.0 | 3.4 |
| Macrophages LPS | 0.5 | 0.4 |
| HUVEC none | 12.2 | 12.8 |
| HUVEC starved | 21.6 | 20.4 |
| HUVEC IL-1beta | 4.9 | 4.3 |
| HUVEC IFN gamma | 19.6 | 19.8 |
| HUVEC TNF alpha + IFN gamma | 7.8 | 8.9 |
| HUVEC TNF alpha + IL4 | 6.1 | 7.9 |
| HUVEC IL-11 | 10.4 | 11.8 |
| Lung Microvascular EC none | 7.5 | 9.8 |
| Lung Microvascular EC TNF alpha + IL-1beta | 5.5 | 6.1 |
| Microvascular Dermal EC none | 13.7 | 12.6 |
| Microsvasular Dermal EC TNFalpha + IL-1beta | 5.7 | 6.6 |
| Bronchial epithelium TNFalpha + IL1beta | 15.9 | 11.9 |
| Small airway epithelium none | 4.7 | 5.3 |
| Small airway epithelium TNFalpha + IL-1beta | 35.6 | 37.1 |
| Coronery artery SMC rest | 7.1 | 6.7 |
| Coronery artery SMC TNFalpha + IL-1beta | 4.6 | 5.9 |
| Astrocytes rest | 27.0 | 23.8 |
| Astrocytes TNFalpha + IL-1beta | 30.8 | 28.1 |
| KU-812 (Basophil) rest | 8.2 | 6.3 |
| KU-812 (Basophil) PMA/ionomycin | 22.5 | 19.9 |
| CCD1106 (Keratinocytes) none | 11.1 | 11.7 |
| CCD1106 (Keratinocytes) TNFalpha + IL-1beta | 6.8 | 6.3 |
| Liver cirrhosis | 3.0 | 3.0 |
| Lupus kidney | 6.5 | 6.2 |
| NCI-H292 none | 63.3 | 72.7 |
| NCI-H292 IL-4 | 57.4 | 69.7 |
| NCI-H292 IL-9 | 57.0 | 65.5 |
| NCI-H292 IL-13 | 30.8 | 35.6 |
| NCI-H292 IFN gamma | 29.3 | 34.4 |
| HPAEC none | 10.8 | 11.7 |
| HPAEC TNF alpha + IL-1 beta | 6.9 | 6.4 |
| Lung fibroblast none | 16.8 | 17.4 |
| Lung fibroblast TNF alpha + IL-1 beta | 7.4 | 8.0 |
| Lung fibroblast IL-4 | 30.6 | 34.6 |
| Lung fibroblast IL-9 | 24.8 | 24.1 |
| Lung fibroblast IL-13 | 19.6 | 21.8 |
| Lung fibroblast IFN gamma | 31.4 | 37.6 |
| Dermal fibroblast CCD1070 rest | 10.7 | 12.0 |
| Dermal fibroblast CCD1070 TNF alpha | 20.6 | 21.3 |
| Dermal fibroblast CCD1070 IL-1 beta | 6.5 | 5.9 |
| Dermal fibroblast IFN gamma | 10.1 | 11.3 |
| Dermal fibroblast IL-4 | 23.0 | 23.2 |
| IBD Colitis 2 | 2.0 | 2.3 |
| IBD Crohn's | 3.4 | 4.8 |
| Colon | 41.5 | 50.7 |
| Lung | 15.8 | 17.2 |
| Thymus | 57.8 | 55.5 |
| Kidney | 5.0 | 8.5 |

Column A - Rel. Exp. (%) Ag2896, Run 164401737
Column B - Rel. Exp. (%) Ag2920, Run 164403312

TABLE JF
Panel 5 Islet

| Tissue Name | A |
|---|---|
| 97457_Patient-02go_adipose | 19.6 |
| 97476_Patient-07sk_skeletal muscle | 6.6 |
| 97477_Patient-07ut_uterus | 31.6 |
| 97478_Patient-07pl_placenta | 2.6 |
| 99167_Bayer Patient 1 | 32.5 |
| 97482_Patient-08ut_uterus | 26.6 |
| 97483_Patient-08pl_placenta | 1.2 |
| 97486_Patient-09sk_skeletal muscle | 8.3 |
| 97487_Patient-09ut_uterus | 47.6 |
| 97488_Patient-09pl_placenta | 1.3 |
| 97492_Patient-10ut_uterus | 28.3 |
| 97493_Patient-10pl_placenta | 3.0 |
| 97495_Patient-11go_adipose | 47.6 |
| 97496_Patient-11sk_skeletal muscle | 35.1 |
| 97497_Patient-11ut_uterus | 100.0 |
| 97498_Patient-11pl_placenta | 0.4 |
| 97500_Patient-12go_adipose | 17.3 |
| 97501_Patient-12sk_skeletal muscle | 37.4 |
| 97502_Patient-12ut_uterus | 76.3 |
| 97503_Patient-12pl_placenta | 0.9 |
| 94721_Donor 2 U - A_Mesenchymal Stem Cells | 15.2 |
| 94722_Donor 2 U - B_Mesenchymal Stem Cells | 11.9 |
| 94723_Donor 2 U - C_Mesenchymal Stem Cells | 9.7 |
| 94709_Donor 2 AM - A_adipose | 8.3 |
| 94710_Donor 2 AM - B_adipose | 8.1 |
| 94711_Donor 2 AM - C_adipose | 5.0 |
| 94712_Donor 2 AD - A_adipose | 17.6 |
| 94713_Donor 2 AD - B_adipose | 29.5 |
| 94714_Donor 2 AD - C_adipose | 25.7 |
| 94742_Donor 3 U - A_Mesenchymal Stem Cells | 5.7 |
| 94743_Donor 3 U - B_Mesenchymal Stem Cells | 5.1 |
| 94730_Donor 3 AM - A_adipose | 8.7 |
| 94731_Donor 3 AM - B_adipose | 4.4 |
| 94732_Donor 3 AM - C_adipose | 3.4 |
| 94733_Donor 3 AD - A_adipose | 9.9 |
| 94734_Donor 3 AD - B_adipose | 4.4 |
| 94735_Donor 3 AD - C_adipose | 4.6 |
| 77138_Liver_HepG2untreated | 54.7 |
| 73556_Heart_Cardiac stromal cells (primary) | 10.6 |
| 81735_Small Intestine | 60.7 |
| 72409_Kidney_Proximal Convoluted Tubule | 24.1 |
| 82685_Small intestine Duodenum | 18.8 |
| 90650_Adrenal_Adrenocortical adenoma | 3.8 |
| 72410_Kidney_HRCE | 70.2 |
| 72411_Kidney_HRE | 59.5 |
| 73139_Uterus_Uterine smooth muscle cells | 8.7 |

Column A - Rel. Exp. (%) Ag2896, Run 268363565

TABLE JG
Panel CNS_1

| Tissue Name | A |
|---|---|
| BA4 Control | 22.1 |
| BA4 Control2 | 41.8 |
| BA4 Alzheimer's2 | 5.2 |
| BA4 Parkinson's | 39.2 |
| BA4 Parkinson's2 | 68.8 |
| BA4 Huntington's | 28.1 |
| BA4 Huntington's2 | 13.9 |
| BA4 PSP | 9.9 |
| BA4 PSP2 | 25.5 |
| BA4 Depression | 22.7 |
| BA4 Depression2 | 6.7 |
| BA7 Control | 34.9 |
| BA7 Control2 | 21.1 |
| BA7 Alzheimer's2 | 6.2 |
| BA7 Parkinson's | 18.3 |
| BA7 Parkinson's2 | 38.2 |
| BA7 Huntington's | 51.1 |
| BA7 Huntington's2 | 38.7 |
| BA7 PSP | 44.4 |
| BA7 PSP2 | 18.9 |
| BA7 Depression | 10.5 |
| BA9 Control | 27.9 |
| BA9 Control2 | 83.5 |
| BA9 Alzheimer's | 4.7 |
| BA9 Alzheimer's2 | 12.6 |
| BA9 Parkinson's | 22.5 |
| BA9 Parkinson's2 | 45.4 |
| BA9 Huntington's | 39.2 |
| BA9 Huntington's2 | 20.0 |
| BA9 PSP | 12.1 |
| BA9 PSP2 | 3.9 |
| BA9 Depression | 8.5 |
| BA9 Depression2 | 9.2 |
| BA17 Control | 25.3 |
| BA17 Control2 | 34.6 |
| BA17 Alzheimer's2 | 4.3 |
| BA17 Parkinson's | 20.0 |
| BA17 Parkinson's2 | 28.3 |
| BA17 Huntington's | 24.1 |
| BA17 Huntington's2 | 12.1 |
| BA17 Depression | 8.7 |
| BA17 Depression2 | 16.6 |
| BA17 PSP | 22.4 |
| BA17 PSP2 | 6.5 |
| Sub Nigra Control | 21.3 |
| Sub Nigra Control2 | 18.3 |
| Sub Nigra Alzheimer's2 | 7.2 |
| Sub Nigra Parkinson's2 | 27.7 |
| Sub Nigra Huntington's | 25.5 |
| Sub Nigra Huntington's2 | 13.6 |
| Sub Nigra PSP2 | 3.4 |
| Sub Nigra Depression | 6.3 |
| Sub Nigra Depression2 | 6.1 |
| Glob Palladus Control | 18.9 |
| Glob Palladus Control2 | 19.9 |
| Glob Palladus Alzheimer's | 7.2 |
| Glob Palladus Alzheimer's2 | 9.8 |
| Glob Palladus Parkinson's | 100.0 |
| Glob Palladus Parkinson's2 | 20.9 |
| Glob Palladus PSP | 13.8 |
| Glob Palladus PSP2 | 12.4 |
| Glob Palladus Depression | 7.5 |
| Temp Pole Control | 20.0 |
| Temp Pole Control2 | 66.9 |
| Temp Pole Alzheimer's | 6.1 |
| Temp Pole Alzheimer's2 | 6.6 |
| Temp Pole Parkinson's | 34.6 |
| Temp Pole Parkinson's2 | 24.0 |
| Temp Pole Huntington's | 33.4 |
| Temp Pole PSP | 8.4 |
| Temp Pole PSP2 | 6.4 |
| Temp Pole Depression2 | 6.7 |
| Cing Gyr Control | 53.6 |
| Cing Gyr Control2 | 34.6 |
| Cing Gyr Alzheimer's | 15.9 |
| Cing Gyr Alzheimer's2 | 12.2 |
| Cing Gyr Parkinson's | 24.5 |
| Cing Gyr Parkinson's2 | 30.8 |
| Cing Gyr Huntington's | 48.0 |
| Cing Gyr Huntington's2 | 16.4 |
| Cing Gyr PSP | 15.2 |
| Cing Gyr PSP2 | 6.0 |
| Cing Gyr Depression | 7.3 |
| Cing Gyr Depression2 | 11.7 |

Column A - Rel. Exp. (%) Ag2896, Run 171688452

CNS_neurodegeneration_v1.0 Summary: Data obtained from RTQPCR assays performed for this panel was analyized using multivariate analysis (ANOVA). The multivariate results obtained analyzing both Ag2896/Ag2920 indicates this gene is found to be down-regulated in the temporal cortex of Alzheimer's disease patients. Therefore, up-regulation of this gene or its gene product, or treatment with specific agonists for this receptor may be of use in treating dementia/memory loss associated with this disease and neuronal death.

Panel 1.3D Summary: Ag2896/Ag2920 Two experiments produce results that are in excellent agreement, with highest expression of this gene in the brain cerebral cortex (CTs=26). High expression of this gene is seen mainly in all the regions of the central nervous system examined, including amygdala, hippocampus, substantia nigra, thalamus, cerebellum, cerebral cortex, and spinal cord. This gene encodes a Ca binding transporter. Ca++ is critical for synaptic vesicle release (Kovacs I, Szarics E, Nyitrai G, Blandl T, Kardos J. Matching kinetics of synaptic vesicle recycling and enhanced neurotransmitter influx by Ca2+ in brain plasma membrane vesicles. Neurochem Int November 1998;33(5):399-405). Thus, this gene would be an excellent small molecule target for diseases resulting from altered/inappropriate synaptic transmission such as epilepsy, schizophrenia, bipolar disorder, depression, and mania.

This gene also has moderate levels of expression adult and fetal heart, skeletal muscle and liver, and adipose. This gene product is homologous to a mitochondrial calcium-dependent transporter. Since intracellular calcium homeostasis is critically important for energy metabolism and signal transduction, modulation of this gene product may therefore be a therapeutic for metabolic and endocrine diseases.

Panel 4D Summary: Ag2896/Ag2920 Two experiments show moderate to low expression of this gene across a wide range of cells of this panel including epithelium, fibroblasts, and endothelial cells. Lower but still significant levels of expression are also seen in the key players of innate and adaptive immunity: monocytes/macrophages, T and B cells. However, the expression of this transcript is highest in the B lymphoma cell line, and NCI H292, a mucoepidermoid cell line (CTs=26.4-27). Thus, inhibition of the function of the protein encoded by this transcript with a small molecule drug, could lead to improvement of the symptoms of patients suffering from autoimmune and inflammatory diseases such as asthma, COPD, emphysema, psoriasis, inflammatory bowel disease, lupus erythematosus, or rheumatoid arthritis.

Panel 5 Islet Summary: Ag2896 This gene shows widespread expression in this panel with highest expression seen in uterus from a non-diabetic patient (CT=28.8). Significant expression of this gene is seen in adipose, skeletal muscle, uterus, kidney, small intestine and a liver cancer cell line, which is in agreement with expression seen in panel 1.3D. Please see panel 1.3D for further discussion on the role of this gene.

Panel CNS_{—}1 Summary: Ag2896 This expression profile confirms the presence of this gene in the brain.

K CG56829-01: Human Testis Specific Serine Kinase-3

Expression of gene CG56829-01 was assessed using the primer-probe sets Ag1301b, Ag1415 and Ag3031, described in Tables KA, KB and KC. Results of the RTQ-PCR runs are shown in Tables KD, KE, KF and KG.

TABLE KA

Probe Name Ag1301b

| | | | | |
|---|---|---|---|---|
| | | | Start | SEQ ID |
| Primers | Sequences | Length | Position | No |

| | | | | |
|---|---|---|---|---|
| Forward | 5′-aaaaggtgatgtctggagcat-3′ | 21 | 616 | 131 |
| Probe | TET-5′-tgtatgtcatgctctgtgccagccta-3′-TAMRA | 26 | 648 | 132 |
| Reverse | 5′-gatgtctgtgtcgtcaaaagg-3′ | 21 | 674 | 133 |

TABLE KB

Probe Name Ag1415

| | | | | |
|---|---|---|---|---|
| | | | Start | SEQ ID |
| Primers | Sequences | Length | Position | No |

| | | | | |
|---|---|---|---|---|
| Forward | 5′-aaaaggtgatgtctggagcat-3′ | 21 | 616 | 134 |
| Probe | TET-5′-tgtatgtcatgctctgtgccagccta-3′-TAMRA | 26 | 648 | 135 |
| Reverse | 5′-gatgtctgtgtcgtcaaaagg-3′ | 21 | 674 | 136 |

TABLE KC

Probe Name Ag3031

| | | | | |
|---|---|---|---|---|
| | | | Start | SEQ ID |
| Primers | Sequences | Length | Position | No |

| | | | | |
|---|---|---|---|---|
| Forward | 5′-aaaaggtgatgtctggagcat-3′ | 21 | 616 | 137 |
| Probe | TET-5′-tgtatgtcatgctctgtgccagccta-3′-TAMRA | 26 | 648 | 138 |
| Reverse | 5′-gatgtctgtgtcgtcaaaagg-3′ | 21 | 674 | 139 |

TABLE KD
CNS_neurodegeneration_v1.0

| Tissue Name | A | B |
|---|---|---|
| AD 1 Hippo | 23.8 | 46.7 |
| AD 2 Hippo | 21.2 | 21.9 |
| AD 3 Hippo | 14.2 | 11.6 |
| AD 4 Hippo | 7.4 | 10.5 |
| AD 5 hippo | 94.0 | 94.0 |
| AD 6 Hippo | 53.6 | 61.6 |
| Control 2 Hippo | 12.7 | 17.0 |
| Control 4 Hippo | 14.6 | 15.2 |
| Control (Path) 3 Hippo | 22.2 | 7.2 |
| AD 1 Temporal Ctx | 42.3 | 42.9 |
| AD 2 Temporal Ctx | 78.5 | 29.9 |
| AD 3 Temporal Ctx | 17.9 | 20.4 |
| AD 4 Temporal Ctx | 30.1 | 31.2 |
| AD 5 Inf Temporal Ctx | 100.0 | 94.6 |
| AD 5 Sup Temporal Ctx | 59.9 | 52.5 |
| AD 6 Inf Temporal Ctx | 75.3 | 95.9 |
| AD 6 Sup Temporal Ctx | 94.6 | 100.0 |
| Control 1 Temporal Ctx | 8.5 | 7.3 |
| Control 2 Temporal Ctx | 16.3 | 18.8 |
| Control 3 Temporal Ctx | 13.0 | 15.3 |
| Control 4 Temporal Ctx | 12.8 | 16.6 |
| Control (Path) 1 Temporal Ctx | 26.8 | 31.0 |
| Control (Path) 2 Temporal Ctx | 25.5 | 17.8 |
| Control (Path) 3 Temporal Ctx | 6.0 | 10.2 |
| Control (Path) 4 Temporal Ctx | 26.4 | 28.1 |
| AD 1 Occipital Ctx | 31.9 | 41.8 |
| AD 2 Occipital Ctx (Missing) | 0.0 | 0.0 |
| AD 3 Occipital Ctx | 17.9 | 17.4 |
| AD 4 Occipital Ctx | 12.9 | 17.3 |
| AD 5 Occipital Ctx | 35.1 | 20.9 |
| AD 6 Occipital Ctx | 20.9 | 27.5 |
| Control 1 Occipital Ctx | 4.9 | 4.9 |
| Control 2 Occipital Ctx | 46.7 | 32.1 |
| Control 3 Occipital Ctx | 17.8 | 19.2 |
| Control 4 Occipital Ctx | 13.2 | 12.1 |
| Control (Path) 1 Occipital Ctx | 49.3 | 59.9 |
| Control (Path) 2 Occipital Ctx | 13.6 | 8.8 |
| Control (Path) 3 Occipital Ctx | 6.1 | 4.7 |
| Control (Path) 4 Occipital Ctx | 31.2 | 21.2 |
| Control 1 Parietal Ctx | 10.3 | 11.1 |
| Control 2 Parietal Ctx | 56.6 | 60.7 |
| Control 3 Parietal Ctx | 15.4 | 15.0 |
| Control (Path) 1 Parietal Ctx | 34.2 | 41.8 |
| Control (Path) 2 Parietal Ctx | 15.4 | 20.3 |
| Control (Path) 3 Parietal Ctx | 8.4 | 7.0 |
| Control (Path) 4 Parietal Ctx | 44.1 | 31.6 |

Column A - Rel. Exp. (%) Ag3031, Run 211011868
Column B - Rel. Exp. (%) Ag3031, Run 225437445

TABLE KE
Panel 1.3D

| Tissue Name | A | B |
|---|---|---|
| Liver adenocarcinoma | 18.4 | 32.3 |
| Pancreas | 4.7 | 11.9 |
| Pancreatic ca. CAPAN 2 | 4.9 | 4.9 |
| Adrenal gland | 23.0 | 11.0 |
| Thyroid | 7.1 | 8.5 |
| Salivary gland | 10.1 | 13.7 |
| Pituitary gland | 18.2 | 9.2 |
| Brain (fetal) | 24.0 | 42.3 |
| Brain (whole) | 43.8 | 19.6 |
| Brain (amygdala) | 20.4 | 9.6 |
| Brain (cerebellum) | 50.7 | 50.3 |
| Brain (hippocampus) | 18.7 | 8.1 |
| Brain (substantia nigra) | 8.4 | 8.9 |
| Brain (thalamus) | 20.0 | 10.6 |
| Cerebral Cortex | 5.3 | 7.2 |
| Spinal cord | 9.6 | 7.0 |
| glio/astro U87-MG | 12.1 | 16.5 |
| glio/astro U-118-MG | 15.5 | 13.4 |
| astrocytoma SW1783 | 6.9 | 11.1 |
| neuro*; met SK-N-AS | 11.4 | 10.9 |
| astrocytoma SF-539 | 12.5 | 25.5 |
| astrocytoma SNB-75 | 10.9 | 18.9 |
| glioma SNB-19 | 32.1 | 25.3 |
| glioma U251 | 100.0 | 100.0 |
| glioma SF-295 | 18.4 | 46.0 |
| Heart (fetal) | 2.3 | 6.2 |
| Heart | 8.4 | 8.2 |
| Skeletal muscle (fetal) | 6.3 | 24.1 |
| Skeletal muscle | 20.2 | 7.9 |
| Bone marrow | 21.8 | 25.2 |
| Thymus | 18.2 | 38.4 |
| Spleen | 26.6 | 14.0 |
| Lymph node | 42.6 | 23.7 |
| Colorectal | 16.8 | 16.8 |
| Stomach | 37.4 | 5.9 |
| Small intestine | 36.6 | 14.2 |
| Colon ca. SW480 | 3.7 | 4.5 |
| Colon ca.* SW620 (SW480 met) | 3.8 | 24.0 |
| Colon ca. HT29 | 1.6 | 4.6 |
| Colon ca. HCT-116 | 3.0 | 9.9 |
| Colon ca. CaCo-2 | 2.1 | 5.1 |
| Colon ca. tissue (ODO3866) | 5.4 | 3.6 |
| Colon ca. HCC-2998 | 6.2 | 15.6 |
| Gastric ca.* (liver met) NCI-N87 | 20.0 | 13.3 |
| Bladder | 8.4 | 12.2 |
| Trachea | 13.6 | 6.4 |
| Kidney | 10.4 | 23.8 |
| Kidney (fetal) | 10.7 | 66.0 |
| Renal ca. 786-0 | 15.3 | 25.5 |
| Renal ca. A498 | 11.9 | 12.5 |
| Renal ca. RXF 393 | 14.9 | 18.0 |
| Renal ca. ACHN | 8.0 | 10.5 |
| Renal ca. UO-31 | 6.0 | 1.1 |
| Renal ca. TK-10 | 7.4 | 18.6 |
| Liver | 11.9 | 17.8 |
| Liver (fetal) | 20.6 | 14.7 |
| Liver ca. (hepatoblast) HepG2 | 22.2 | 28.7 |
| Lung | 17.0 | 22.8 |
| Lung (fetal) | 10.4 | 26.4 |
| Lung ca. (small cell) LX-1 | 9.1 | 17.4 |
| Lung ca. (small cell) NCI-H69 | 1.5 | 4.0 |
| Lung ca. (s. cell var.) SHP-77 | 18.4 | 57.4 |
| Lung ca. (large cell) NCI-H460 | 50.3 | 4.2 |
| Lung ca. (non-sm. cell) A549 | 8.0 | 28.1 |
| Lung ca. (non-s. cell) NCI-H23 | 10.2 | 18.2 |
| Lung ca. (non-s. cell) HOP-62 | 20.3 | 33.0 |
| Lung ca. (non-s. cl) NCI-H522 | 9.7 | 23.5 |
| Lung ca. (squam.) SW 900 | 4.0 | 8.5 |
| Lung ca. (squam.) NCI-H596 | 1.6 | 4.8 |
| Mammary gland | 36.9 | 22.5 |
| Breast ca.* (pl. ef) MCF-7 | 5.4 | 6.7 |
| Breast ca.* (pl. ef) MDA-MB-231 | 16.7 | 7.9 |
| Breast ca.* (pl. ef) T47D | 13.2 | 46.7 |
| Breast ca. BT-549 | 9.9 | 6.6 |
| Breast ca. MDA-N | 1.6 | 11.9 |
| Ovary | 3.0 | 5.1 |
| Ovarian ca. OVCAR-3 | 5.1 | 12.0 |
| Ovarian ca. OVCAR-4 | 4.7 | 8.8 |
| Ovarian ca. OVCAR-5 | 8.3 | 42.3 |
| Ovarian ca. OVCAR-8 | 2.8 | 0.9 |
| Ovarian ca. IGROV-1 | 2.6 | 11.7 |
| Ovarian ca.* (ascites) SK-OV-3 | 10.0 | 29.9 |
| Uterus | 38.4 | 17.4 |
| Placenta | 11.1 | 9.4 |
| Prostate | 18.6 | 8.5 |
| Prostate ca.* (bone met) PC-3 | 6.9 | 14.1 |
| Testis | 45.1 | 26.6 |
| Melanoma Hs688(A).T | 0.8 | 1.2 |
| Melanoma* (met) Hs688(B).T | 3.8 | 3.4 |
| Melanoma UACC-62 | 4.6 | 8.0 |
| Melanoma M14 | 28.5 | 9.9 |
| Melanoma LOX IMVI | 0.7 | 8.1 |
| Melanoma* (met) SK-MEL-5 | 2.2 | 2.9 |
| Adipose | 8.1 | 12.3 |

Column A - Rel. Exp. (%) Ag1301b, Run 165528224
Column B - Rel. Exp. (%) Ag3031, Run 167961982

TABLE KF
Panel 2.2

| Tissue Name | A |
|---|---|
| Normal Colon | 39.2 |
| Colon cancer (OD06064) | 6.3 |
| Colon Margin (OD06064) | 13.0 |
| Colon cancer (OD06159) | 0.0 |
| Colon Margin (OD06159) | 27.0 |
| Colon cancer (OD06297-04) | 2.5 |
| Colon Margin (OD06297-05) | 39.8 |
| CC Gr.2 ascend colon (ODO3921) | 3.8 |
| CC Margin (ODO3921) | 4.4 |
| Colon cancer metastasis (OD06104) | 14.3 |
| Lung Margin (OD06104) | 17.2 |
| Colon mets to lung (OD04451-01) | 0.0 |
| Lung Margin (OD04451-02) | 10.2 |
| Normal Prostate | 30.6 |
| Prostate Cancer (OD04410) | 7.6 |
| Prostate Margin (OD04410) | 25.5 |
| Normal Ovary | 25.0 |
| Ovarian cancer (OD06283-03) | 8.1 |
| Ovarian Margin (OD06283-07) | 34.4 |
| Ovarian Cancer 064008 | 46.3 |
| Ovarian cancer (OD06145) | 34.4 |
| Ovarian Margin (OD06145) | 52.9 |
| Ovarian cancer (OD06455-03) | 11.6 |
| Ovarian Margin (OD06455-07) | 18.8 |
| Normal Lung | 22.8 |
| Invasive poor diff. lung adeno (ODO4945-01 | 18.7 |
| Lung Margin (ODO4945-03) | 13.9 |
| Lung Malignant Cancer (OD03126) | 11.7 |
| Lung Margin (OD03126) | 12.2 |
| Lung Cancer (OD05014A) | 9.0 |
| Lung Margin (OD05014B) | 35.4 |
| Lung cancer (OD06081) | 23.7 |
| Lung Margin (OD06081) | 27.9 |
| Lung Cancer (OD04237-01) | 13.3 |
| Lung Margin (OD04237-02) | 28.5 |
| Ocular Melanoma Metastasis | 11.5 |
| Ocular Melanoma Margin (Liver) | 27.0 |
| Melanoma Metastasis | 21.6 |
| Melanoma Margin (Lung) | 14.7 |
| Normal Kidney | 17.8 |
| Kidney Ca, Nuclear grade 2 (OD04338) | 50.3 |
| Kidney Margin (OD04338) | 24.3 |
| Kidney Ca Nuclear grade 1/2 (OD04339) | 62.0 |
| Kidney Margin (OD04339) | 16.3 |
| Kidney Ca, Clear cell type (OD04340) | 7.3 |
| Kidney Margin (OD04340) | 10.7 |
| Kidney Ca, Nuclear grade 3 (OD04348) | 4.6 |
| Kidney Margin (OD04348) | 79.0 |
| Kidney malignant cancer (OD06204B) | 6.0 |
| Kidney normal adjacent tissue (OD06204E) | 15.4 |
| Kidney Cancer (OD04450-01) | 24.5 |
| Kidney Margin (OD04450-03) | 23.7 |
| Kidney Cancer 8120613 | 3.0 |
| Kidney Margin 8120614 | 38.2 |
| Kidney Cancer 9010320 | 8.0 |
| Kidney Margin 9010321 | 10.8 |
| Kidney Cancer 8120607 | 15.2 |
| Kidney Margin 8120608 | 3.8 |
| Normal Uterus | 33.7 |
| Uterine Cancer 064011 | 33.9 |
| Normal Thyroid | 2.9 |
| Thyroid Cancer 064010 | 4.4 |
| Thyroid Cancer A302152 | 29.9 |
| Thyroid Margin A302153 | 7.3 |
| Normal Breast | 37.6 |
| Breast Cancer (OD04566) | 21.6 |
| Breast Cancer 1024 | 100.0 |
| Breast Cancer (OD04590-01) | 25.2 |
| Breast Cancer Mets (OD04590-03) | 35.6 |
| Breast Cancer Metastasis (OD04655-05) | 45.1 |
| Breast Cancer 064006 | 21.2 |
| Breast Cancer 9100266 | 26.6 |
| Breast Margin 9100265 | 19.3 |
| Breast Cancer A209073 | 6.1 |
| Breast Margin A2090734 | 35.8 |
| Breast cancer (OD06083) | 49.3 |
| Breast cancer node metastasis (OD06083) | 25.9 |
| Normal Liver | 36.3 |
| Liver Cancer 1026 | 2.5 |
| Liver Cancer 1025 | 45.7 |
| Liver Cancer 6004-T | 30.1 |
| Liver Tissue 6004-N | 27.7 |
| Liver Cancer 6005-T | 6.7 |
| Liver Tissue 6005-N | 17.4 |
| Liver Cancer 064003 | 32.3 |
| Normal Bladder | 13.2 |
| Bladder Cancer 1023 | 23.0 |
| Bladder Cancer A302173 | 20.0 |
| Normal Stomach | 89.5 |
| Gastric Cancer 9060397 | 5.7 |
| Stomach Margin 9060396 | 17.7 |
| Gastric Cancer 9060395 | 19.6 |
| Stomach Margin 9060394 | 42.6 |
| Gastric Cancer 064005 | 7.5 |

Column A - Rel. Exp. (%) Ag1301b, Run 173859869

TABLE KG
Panel 4D

| Tissue Name | A | B | C |
|---|---|---|---|
| Secondary Th1 act | 17.0 | 25.3 | 13.8 |
| Secondary Th2 act | 22.7 | 18.9 | 20.0 |
| Secondary Tr1 act | 32.3 | 26.4 | 20.0 |
| Secondary Th1 rest | 22.2 | 20.3 | 12.0 |
| Secondary Th2 rest | 42.6 | 37.9 | 21.0 |
| Secondary Tr1 rest | 27.9 | 27.5 | 26.8 |
| Primary Th1 act | 33.4 | 29.9 | 19.1 |
| Primary Th2 act | 28.1 | 41.2 | 13.3 |
| Primary Tr1 act | 49.0 | 52.5 | 20.9 |
| Primary Th1 rest | 80.7 | 87.1 | 80.7 |
| Primary Th2 rest | 67.4 | 68.8 | 64.2 |
| Primary Tr1 rest | 46.0 | 50.3 | 54.7 |
| CD45RA CD4 lymphocyte act | 12.9 | 8.0 | 16.5 |
| CD45RO CD4 lymphocyte act | 33.4 | 44.8 | 22.4 |
| CD8 lymphocyte act | 22.8 | 23.0 | 18.4 |
| Secondary CD8 lymphocyte rest | 22.2 | 25.5 | 25.0 |
| Secondary CD8 lymphocyte act | 19.3 | 21.8 | 22.5 |
| CD4 lymphocyte none | 41.5 | 42.3 | 34.6 |
| 2ry Th1/Th2/Tr1_anti-CD95 CH11 | 65.1 | 54.7 | 41.2 |
| LAK cells rest | 28.7 | 37.1 | 32.3 |
| LAK cells IL-2 | 38.7 | 49.0 | 36.3 |
| LAK cells IL-2 + IL-12 | 26.8 | 27.4 | 26.8 |
| LAK cells IL-2 + IFN gamma | 43.5 | 45.4 | 42.9 |
| LAK cells IL-2 + IL-18 | 26.2 | 25.2 | 35.6 |
| LAK cells PMA/ionomycin | 8.3 | 8.6 | 3.3 |
| NK Cells IL-2 rest | 28.7 | 35.8 | 32.8 |
| Two Way MLR 3 day | 42.3 | 49.0 | 46.3 |
| Two Way MLR 5 day | 17.9 | 16.2 | 13.5 |
| Two Way MLR 7 day | 14.7 | 12.6 | 14.4 |
| PBMC rest | 21.9 | 29.9 | 19.2 |
| PBMC PWM | 66.9 | 53.2 | 50.3 |
| PBMC PHA-L | 35.6 | 46.7 | 23.3 |
| Ramos (B cell) none | 23.5 | 33.2 | 16.5 |
| Ramos (B cell) ionomycin | 53.2 | 53.2 | 49.3 |
| B lymphocytes PWM | 29.9 | 36.3 | 34.2 |
| B lymphocytes CD40L and IL-4 | 33.2 | 36.6 | 35.8 |
| EOL-1 dbcAMP | 18.9 | 14.8 | 12.8 |
| EOL-1 dbcAMP | 30.4 | 29.1 | 25.5 |
| PMA/ionomycin | | | |
| Dendritic cells none | 14.9 | 10.0 | 13.7 |
| Dendritic cells LPS | 15.7 | 5.8 | 8.7 |
| Dendritic cells anti-CD40 | 12.7 | 17.7 | 15.2 |
| Monocytes rest | 35.6 | 27.5 | 36.6 |
| Monocytes LPS | 34.6 | 43.8 | 25.0 |
| Macrophages rest | 19.2 | 16.6 | 17.2 |
| Macrophages LPS | 17.9 | 16.2 | 6.7 |
| HUVEC none | 9.8 | 13.2 | 17.3 |
| HUVEC starved | 27.2 | 27.5 | 28.3 |
| HUVEC IL-1beta | 11.5 | 13.0 | 11.3 |
| HUVEC IFN gamma | 28.3 | 24.7 | 20.2 |
| HUVEC TNF alpha + IFN gamma | 3.2 | 6.6 | 12.3 |
| HUVEC TNF alpha + IL4 | 11.7 | 9.7 | 9.7 |
| HUVEC IL- 11 | 10.1 | 8.2 | 11.6 |
| Lung Microvascular EC none | 33.9 | 28.7 | 25.2 |
| Lung Microvascular EC TNFalpha + IL- | 23.2 | 19.1 | 24.8 |
| 1beta | | | |
| Microvascular Dermal EC none | 41.8 | 45.1 | 25.7 |
| Microsvasular Dermal EC TNFalpha + | 27.5 | 40.3 | 22.4 |
| IL-1beta | | | |
| Bronchial epithelium TNFalpha + | 20.3 | 32.3 | 22.1 |
| IL1beta | | | |
| Small airway epithelium none | 7.3 | 3.3 | 4.8 |
| Small airway epithelium TNFalpha + | 34.4 | 35.1 | 26.8 |
| IL-1beta | | | |
| Coronery artery SMC rest | 7.9 | 7.9 | 12.3 |
| Coronery artery SMC TNFalpha + IL- | 7.6 | 10.7 | 8.1 |
| 1beta | | | |
| Astrocytes rest | 7.9 | 10.3 | 12.7 |
| Astrocytes TNFalpha + IL-1beta | 8.7 | 5.8 | 13.3 |
| KU-812 (Basophil) rest | 40.1 | 35.4 | 48.0 |
| KU-812 (Basophil) PMA/ionomycin | 57.8 | 61.1 | 78.5 |
| CCD1106 (Keratinocytes) none | 3.8 | 8.0 | 6.6 |
| CCD1106 (Keratinocytes) TNFalpha + | 27.4 | 24.5 | 5.3 |
| IL-1beta | | | |
| Liver cirrhosis | 17.0 | 9.4 | 5.0 |
| Lupus kidney | 24.1 | 23.8 | 9.5 |
| NCI-H292 none | 38.7 | 49.3 | 45.4 |
| NCI-H292 IL-4 | 58.6 | 51.4 | 46.7 |
| NCI-H292 IL-9 | 56.3 | 46.0 | 54.3 |
| NCI-H292 IL-13 | 30.4 | 31.9 | 23.3 |
| NCI-H292 IFN gamma | 16.7 | 28.3 | 29.1 |
| HPAEC none | 15.7 | 26.4 | 19.1 |
| HPAEC TNF alpha + IL-1 beta | 23.8 | 32.5 | 27.9 |
| Lung fibroblast none | 13.0 | 11.0 | 11.9 |
| Lung fibroblast TNF alpha + IL-1 | 8.7 | 7.2 | 13.2 |
| beta | | | |
| Lung fibroblast IL-4 | 5.6 | 12.0 | 10.5 |
| Lung fibroblast IL-9 | 8.5 | 7.4 | 15.3 |
| Lung fibroblast IL-13 | 20.3 | 16.3 | 8.9 |
| Lung fibroblast IFN gamma | 11.1 | 10.4 | 13.0 |
| Dermal fibroblast CCD1070 rest | 47.0 | 11.3 | 13.8 |
| Dermal fibroblast CCD1070 TNF alpha | 45.7 | 53.6 | 55.1 |
| Dermal fibroblast CCD1070 IL-1 beta | 16.8 | 17.3 | 15.4 |
| Dermal fibroblast IFN gamma | 5.8 | 8.8 | 6.5 |
| Dermal fibroblast IL-4 | 17.6 | 20.0 | 13.6 |
| IBD Colitis 2 | 2.0 | 1.3 | 1.1 |
| IBD Crohn's | 3.0 | 1.4 | 1.4 |
| Colon | 33.0 | 26.2 | 34.6 |
| Lung | 6.8 | 13.5 | 7.5 |
| Thymus | 100.0 | 100.0 | 55.5 |
| Kidney | 87.1 | 79.0 | 100.0 |

Column A - Rel. Exp. (%) Ag1301b, Run 138983163
Column B - Rel. Exp. (%) Ag1415, Run 138642033
Column C - Rel. Exp. (%) Ag3031, Run 162426783

CNS_neurodegeneration_v1.0 Summary: Data obtained from RTQPCR assays performed for this panel was analyized using multivariate analysis (ANOVA). The multivariate results obtained analyzing Ag3031 in two experiments with the same probe and primer set produce results that are in excellent agreement. This gene, a kinase homolog, is upregulated in the temporal cortex of brains from Alzheimer's disease patients compared to th expression in temporal cortex of normal brains. Kinases have been shown to play a role in the pathogenesis of Alzheimer's disease (Morishima Y, et al. J Neurosci Oct. 1, 2001;21(19):7551-60). The dysregulation of this kinase, CG56829-01, indicates an active role for this pathway in disease pathogenesis. Thus, inhibitors of this gene or the kinase encoded by this gene may have utility in the treatment of Alzheimer's disease and other neurodegenerative diseases.

Panel 1.3D Summary: Ag1301b/Ag3031 Two experiments with the same probe and primer set produce results that are in excellent agreement, with highest expression of the CG56829-01 gene in a brain cancer cell line (CTs=29-30). Overall, this gene is expressed at moderate to low levels in all the samples in this panel.

This gene has low to moderate expression in several endocrine/metabolic-related tissues, including adipose, pancreas, liver, skeletal muscle and thyroid. Thus, a therapeutic modulator to this gene and/or gene-product may be useful in the treatment of diseases which affect the endocrine system.

Panel 2.2 Summary: Ag1301b The CG56829-01 gene is expressed in breast cancer at a moderate level. It is also expressed at a higher level in normal gastric, prostate and colon tissues compared to the adjacent tumors. Hence, inhibition of this drug might be used for treatment of breast cancer. It could also be used as a diagnostic marker for gastric, prostate and colon cancers.

Panel 4D Summary: Ag1301b/Ag1415/Ag3031 Three experiments with the same probe and primer sets produce results that are in excellent agreement, with highest expression of the CG56829-01 gene in the thymus and kidney. This gene is also expressed at higher levels in resting Th1and Th2 lymphocytes than in activated Th1and Th2 lymphocytes. Therefore, modulation of the gene product with small molecule compounds and biomolecules may be useful as therapeutics to reduce the activation of Th1 and Th2 cells and thus reduce symptoms in patients with autoimmune and inflammatory diseases, such as Crohn's disease, ulcerative colitis, multiple sclerosis, chronic obstructive pulmonary disease, asthma, emphysema, rheumatoid arthritis, lupus erythematosus, or psoriasis.

L. CG57183-01: Fibroblast Growth Factor Receptor

Expression of gene CG57183-01 was assessed using the primer-probe sets Ag4039, Ag4040 and Ag4045, described in Tables LA, LB and LC. Results of the RTQ-PCR runs are shown in Tables LD, LE, LF, LG, LH, LI, LJ and LK.

TABLE LA

Probe Name Ag4039

| | | | | |
|---|---|---|---|---|
| | | | Start | SEQ ID |
| Primers | Sequences | Length | Position | No |

| | | | | |
|---|---|---|---|---|
| Forward | 5′-cctacgttaccgtgctcaag-3′ | 20 | 950 | 140 |
| Probe | TET-5′-cgctaacaccaccgacaaggagctag-3′-TAMRA | 26 | 978 | 141 |
| Reverse | 5′-gacgttgtgcaaggagagaac-3′ | 21 | 1006 | 142 |

TABLE LB

Probe Name Ag4040

| | | | | |
|---|---|---|---|---|
| | | | Start | SEQ ID |
| Primers | Sequences | Length | Position | No |

| | | | | |
|---|---|---|---|---|
| Forward | 5′-cctacgttaccgtgctcaag-3′ | 20 | 950 | 143 |
| Probe | TET-5′-cgctaacaccaccgacaaggagctag-3′-TAMRA | 26 | 978 | 144 |
| Reverse | 5′-gacgttgtgcaaggagagaac-3′ | 21 | 1006 | 145 |

TABLE LC

Probe Name Ag4045

| | | | | |
|---|---|---|---|---|
| | | | Start | SEQ ID |
| Primers | Sequences | Length | Position | No |

| | | | | |
|---|---|---|---|---|
| Forward | 5′-tcaccgtagccgtgaagat-3′ | 19 | 1547 | 146 |
| Probe | TET-5′-aaagacgatgccactgacaaggacct-3′-TAMRA | 26 | 1570 | 147 |
| Reverse | 5′-ttcatcatctccatctcagaca-3′ | 22 | 1607 | 148 |

TABLE LD
A1.05 chondrosarcoma

| Tissue Name | A | B |
|---|---|---|
| 138353_PMA (18 hrs) | 17.9 | 5.6 |
| 138352_IL-1beta + Oncostatin M (18 hrs) | 27.4 | 2.4 |
| 138351_IL-1beta + TNFa (18 hrs) | 12.5 | 0.4 |
| 138350_IL-1beta (18 hrs) | 1.0 | 11.8 |
| 138354_Untreated-complete medium (18 hrs) | 15.0 | 6.3 |
| 138347_PMA (6 hrs) | 54.0 | 73.2 |
| 138346_IL-1beta + Oncostatin M (6 hrs) | 43.5 | 50.0 |
| 138345_IL-1beta + TNFa (6 hrs) | 42.3 | 30.8 |
| 138344_IL-1beta (6 hrs) | 51.1 | 22.5 |
| 138348_Untreated-complete medium (6 hrs) | 41.8 | 54.7 |
| 138349_Untreated-serum starved (6 hrs) | 100.0 | 100.0 |

Column A - Rel. Exp. (%) Ag4039, Run 316264543
Column B - Rel. Exp. (%) Ag4045, Run 306518772

TABLE LE
A1_comprehensive panel_v1.0

| Tissue Name | A | B | C |
|---|---|---|---|
| 110967 COPD-F | 0.0 | 0.0 | 0.0 |
| 110980 COPD-F | 0.6 | 0.0 | 0.6 |
| 110968 COPD-M | 0.7 | 1.5 | 0.2 |
| 110977 COPD-M | 2.1 | 0.0 | 0.4 |
| 110989 Emphysema-F | 1.0 | 2.4 | 1.8 |
| 110992 Emphysema-F | 1.3 | 0.9 | 0.7 |
| 110993 Emphysema-F | 0.4 | 0.6 | 0.3 |
| 110994 Emphysema-F | 0.4 | 0.2 | 0.0 |
| 110995 Emphysema-F | 0.0 | 1.8 | 2.3 |
| 110996 Emphysema-F | 0.0 | 0.5 | 0.4 |
| 110997 Asthma-M | 0.2 | 0.0 | 1.7 |
| 111001 Asthma-F | 1.2 | 0.8 | 0.8 |
| 111002 Asthma-F | 1.1 | 3.4 | 1.6 |
| 111003 Atopic Asthma-F | 2.0 | 1.0 | 2.6 |
| 111004 Atopic Asthma-F | 2.6 | 2.3 | 10.3 |
| 111005 Atopic Asthma-F | 1.0 | 1.0 | 9.9 |
| 111006 Atopic Asthma-F | 1.1 | 0.0 | 0.6 |
| 111417 Allergy-M | 0.0 | 0.5 | 3.3 |
| 112347 Allergy-M | 0.0 | 0.0 | 0.0 |
| 112349 Normal Lung-F | 0.0 | 0.0 | 0.0 |
| 112357 Normal Lung-F | 0.1 | 0.6 | 1.2 |
| 112354 Normal Lung-M | 0.4 | 0.0 | 0.0 |
| 112374 Crohns-F | 0.0 | 0.5 | 0.9 |
| 112389 Match Control Crohns-F | 0.5 | 0.6 | 61.6 |
| 112375 Crohns-F | 1.6 | 1.1 | 1.0 |
| 112732 Match Control Crohns-F | 1.6 | 0.7 | 49.0 |
| 112725 Crohns-M | 0.0 | 0.4 | 0.0 |
| 112387 Match Control Crohns-M | 0.8 | 1.3 | 0.4 |
| 112378 Crohns-M | 0.0 | 0.0 | 0.0 |
| 112390 Match Control Crohns-M | 0.1 | 1.4 | 1.4 |
| 112726 Crohns-M | 1.5 | 1.8 | 4.2 |
| 112731 Match Control Crohns-M | 0.2 | 1.4 | 3.6 |
| 112380 Ulcer Col-F | 2.5 | 1.2 | 5.8 |
| 112734 Match Control Ulcer Col-F | 1.6 | 2.1 | 62.4 |
| 112384 Ulcer Col-F | 1.4 | 1.8 | 1.0 |
| 112737 Match Control Ulcer Col-F | 1.1 | 1.1 | 1.5 |
| 112386 Ulcer Col-F | 0.0 | 0.2 | 1.0 |
| 112738 Match Control Ulcer Col-F | 0.5 | 1.2 | 4.5 |
| 112381 Ulcer Col-M | 0.0 | 0.0 | 0.0 |
| 112735 Match Control Ulcer Col-M | 0.0 | 0.5 | 0.1 |
| 112382 Ulcer Col-M | 0.1 | 0.0 | 19.1 |
| 112394 Match Control Ulcer Col-M | 0.4 | 0.0 | 0.1 |
| 112383 Ulcer Col-M | 2.3 | 2.9 | 3.6 |
| 112736 Match Control Ulcer Col-M | 0.2 | 0.2 | 19.2 |
| 112423 Psoriasis-F | 0.0 | 0.0 | 0.2 |
| 112427 Match Control Psoriasis-F | 1.9 | 2.8 | 1.4 |
| 112418 Psoriasis-M | 0.2 | 0.0 | 0.1 |
| 112723 Match Control Psoriasis-M | 0.5 | 0.0 | 0.7 |
| 112419 Psoriasis-M | 0.0 | 0.5 | 0.1 |
| 112424 Match Control Psoriasis-M | 0.5 | 0.6 | 0.0 |
| 112420 Psoriasis-M | 1.4 | 2.0 | 1.9 |
| 112425 Match Control Psoriasis-M | 2.4 | 1.1 | 1.5 |
| 104689 (MF) OA Bone-Backus | 52.5 | 63.7 | 65.1 |
| 104690 (MF) Adj “Normal” Bone-Backus | 14.1 | 18.3 | 23.8 |
| 104691 (MF) OA Synovium-Backus | 0.3 | 1.1 | 0.9 |
| 104692 (BA) OA Cartilage-Backus | 100.0 | 83.5 | 94.0 |
| 104694 (BA) OA Bone-Backus | 83.5 | 100.0 | 100.0 |
| 104695 (BA) Adj “Normal” Bone-Backus | 55.9 | 58.6 | 51.4 |
| 104696 (BA) OA Synovium-Backus | 2.1 | 1.7 | 0.9 |
| 104700 (SS) OA Bone-Backus | 22.2 | 27.7 | 34.9 |
| 104701 (SS) Adj “Normal” Bone-Backus | 42.0 | 47.3 | 57.0 |
| 104702 (SS) OA Synovium-Backus | 5.8 | 2.8 | 5.9 |
| 117093 OA Cartilage Rep7 | 0.0 | 1.6 | 1.1 |
| 112672 OA Bone5 | 1.0 | 1.3 | 2.4 |
| 112673 OA Synovium5 | 0.0 | 0.0 | 0.9 |
| 112674 OA Synovial Fluid cells5 | 0.0 | 0.3 | 0.3 |
| 117100 OA Cartilage Rep14 | 0.5 | 0.5 | 0.4 |
| 112756 OA Bone9 | 1.1 | 1.5 | 3.1 |
| 112757 OA Synovium9 | 0.8 | 0.8 | 0.0 |
| 112758 OA Synovial Fluid Cells9 | 0.0 | 0.9 | 0.3 |
| 117125 RA Cartilage Rep2 | 0.3 | 0.4 | 0.8 |
| 113492 Bone2 RA | 1.0 | 4.8 | 11.6 |
| 113493 Synovium2 RA | 1.0 | 0.8 | 3.8 |
| 113494 Syn Fluid Cells RA | 2.7 | 1.5 | 7.5 |
| 113499 Cartilage4 RA | 1.1 | 2.0 | 3.8 |
| 113500 Bone4 RA | 0.7 | 0.0 | 5.6 |
| 113501 Synovium4 RA | 0.0 | 1.1 | 3.9 |
| 113502 Syn Fluid Cells4 RA | 0.3 | 0.1 | 3.4 |
| 113495 Cartilage3 RA | 1.1 | 2.5 | 8.3 |
| 113496 Bone3 RA | 2.2 | 1.4 | 9.3 |
| 113497 Synovium3 RA | 1.5 | 0.7 | 3.8 |
| 113498 Syn Fluid Cells3 RA | 4.2 | 0.6 | 10.1 |
| 117106 Normal Cartilage Rep20 | 0.0 | 0.7 | 2.0 |
| 113663 Bone3 Normal | 0.0 | 0.0 | 0.0 |
| 113664 Synovium3 Normal | 0.0 | 0.0 | 0.0 |
| 113665 Syn Fluid Cells3 Normal | 0.0 | 0.0 | 0.0 |
| 117107 Normal Cartilage Rep22 | 0.4 | 0.0 | 0.0 |
| 113667 Bone4 Normal | 0.0 | 0.4 | 0.5 |
| 113668 Synovium4 Normal | 0.5 | 0.2 | 0.1 |
| 113669 Syn Fluid Cells4 Normal | 0.5 | 0.6 | 0.0 |

Column A - Rel. Exp. (%) Ag4039, Run 257315337
Column B - Rel. Exp. (%) Ag4040, Run 257315338
Column C - Rel. Exp. (%) Ag4045, Run 257315365

TABLE KG
Panel 4D

| Tissue Name | A | B | C | D |
|---|---|---|---|---|
| AD 1 Hippo | 25.2 | 31.6 | 23.8 | 19.8 |
| AD 2 Hippo | 61.1 | 98.6 | 64.6 | 58.6 |
| AD 3 Hippo | 9.9 | 9.7 | 11.7 | 23.8 |
| AD 4 Hippo | 34.6 | 26.8 | 33.0 | 39.5 |
| AD 5 hippo | 47.3 | 48.0 | 33.9 | 53.2 |
| AD 6 Hippo | 50.3 | 60.7 | 42.0 | 57.4 |
| Control 2 Hippo | 38.7 | 41.2 | 39.0 | 41.2 |
| Control 4 Hippo | 60.3 | 58.6 | 64.6 | 82.4 |
| Control (Path) 3 Hippo | 24.0 | 20.7 | 13.0 | 18.2 |
| AD 1 Temporal Ctx | 19.2 | 23.5 | 21.3 | 30.6 |
| AD 2 Temporal Ctx | 57.4 | 52.1 | 52.1 | 41.5 |
| AD 3 Temporal Ctx | 10.2 | 11.3 | 10.0 | 12.8 |
| AD 4 Temporal Ctx | 39.0 | 47.3 | 44.4 | 36.9 |
| AD 5 Inf Temporal Ctx | 52.1 | 52.5 | 45.7 | 45.4 |
| AD 5 Sup Temporal Ctx | 42.0 | 52.9 | 33.9 | 58.2 |
| AD 6 Inf Temporal Ctx | 36.6 | 40.9 | 40.9 | 34.2 |
| AD 6 Sup Temporal Ctx | 33.2 | 48.6 | 35.6 | 31.6 |
| Control 1 Temporal Ctx | 37.9 | 31.4 | 33.0 | 32.5 |
| Control 2 Temporal Ctx | 52.1 | 52.9 | 46.3 | 39.8 |
| Control 3 Temporal Ctx | 34.2 | 33.4 | 32.8 | 33.7 |
| Control 4 Temporal Ctx | 44.4 | 50.7 | 40.9 | 46.0 |
| Control (Path) 1 Temporal Ctx | 70.7 | 77.4 | 61.6 | 66.4 |
| Control (Path) 2 Temporal Ctx | 41.2 | 72.2 | 46.0 | 57.0 |
| Control (Path) 3 Temporal Ctx | 20.4 | 27.9 | 26.8 | 26.8 |
| Control (Path) 4 Temporal Ctx | 58.2 | 76.8 | 50.3 | 66.4 |
| AD 1 Occipital Ctx | 23.0 | 24.5 | 13.0 | 20.3 |
| AD 2 Occipital Ctx (Missing) | 0.0 | 0.0 | 0.0 | 0.0 |
| AD 3 Occipital Ctx | 12.6 | 20.3 | 13.2 | 15.6 |
| AD 4 Occipital Ctx | 35.1 | 39.2 | 27.4 | 37.1 |
| AD 5 Occipital Ctx | 26.6 | 42.3 | 16.2 | 31.9 |
| AD 6 Occipital Ctx | 36.3 | 28.1 | 30.4 | 40.1 |
| Control 1 Occipital Ctx | 26.4 | 34.2 | 28.9 | 24.0 |
| Control 2 Occipital Ctx | 52.9 | 57.4 | 60.3 | 73.7 |
| Control 3 Occipital Ctx | 36.9 | 55.9 | 46.7 | 46.3 |
| Control 4 Occipital Ctx | 35.4 | 34.9 | 35.1 | 36.6 |
| Control (Path) 1 Occipital Ctx | 100.0 | 92.0 | 100.0 | 100.0 |
| Control (Path) 2 Occipital Ctx | 25.7 | 27.0 | 25.0 | 28.7 |
| Control (Path) 3 Occipital Ctx | 19.5 | 23.5 | 17.1 | 18.9 |
| Control (Path) 4 Occipital Ctx | 31.9 | 42.3 | 23.3 | 48.6 |
| Control 1 Parietal Ctx | 35.6 | 37.4 | 31.6 | 33.9 |
| Control 2 Parietal Ctx | 30.6 | 58.6 | 31.0 | 51.4 |
| Control 3 Parietal Ctx | 35.8 | 42.0 | 30.8 | 26.4 |
| Control (Path) 1 Parietal Ctx | 79.6 | 100.0 | 67.4 | 73.7 |
| Control (Path) 2 Parietal Ctx | 41.2 | 50.7 | 43.2 | 58.6 |
| Control (Path) 3 Parietal Ctx | 24.8 | 32.3 | 19.9 | 31.6 |
| Control (Path) 4 Parietal Ctx | 76.8 | 90.8 | 49.7 | 85.9 |

Column A - Rel. Exp. (%) Ag4039, Run 214151873
Column B - Rel. Exp. (%) Ag4040, Run 214151886
Column C - Rel. Exp. (%) Ag4040, Run 224341025
Column D - Rel. Exp. (%) Ag4045, Run 214291846

TABLE LG
General_screening_panel_v1.4

| Tissue Name | A | B | C |
|---|---|---|---|
| Adipose | 1.3 | 0.7 | 0.9 |
| Melanoma* Hs688(A).T | 0.2 | 0.8 | 0.2 |
| Melanoma* Hs688(B).T | 1.0 | 1.4 | 0.7 |
| Melanoma* M14 | 0.2 | 0.2 | 0.0 |
| Melanoma* LOXIMVI | 0.5 | 1.3 | 0.4 |
| Melanoma* SK-MEL-5 | 1.5 | 1.5 | 1.8 |
| Squamous cell carcinoma | 0.1 | 0.1 | 16.3 |
| SCC-4 | | | |
| Testis Pool | 7.0 | 5.4 | 4.0 |
| Prostate ca.* | 0.0 | 0.0 | 0.0 |
| (bone met) PC-3 | | | |
| Prostate Pool | 1.5 | 1.2 | 4.9 |
| Placenta | 7.7 | 5.7 | 4.5 |
| Uterus Pool | 0.4 | 0.0 | 1.0 |
| Ovarian ca. OVCAR-3 | 0.0 | 0.0 | 6.0 |
| Ovarian ca. SK-OV-3 | 6.8 | 6.5 | 8.7 |
| Ovarian ca. OVCAR-4 | 0.0 | 0.0 | 3.1 |
| Ovarian ca. OVCAR-5 | 18.4 | 17.8 | 42.9 |
| Ovarian ca. IGROV-1 | 42.0 | 22.8 | 14.6 |
| Ovarian ca. OVCAR-8 | 26.2 | 26.4 | 13.9 |
| Ovary | 0.0 | 0.2 | 0.6 |
| Breast ca. MCF-7 | 0.0 | 0.0 | 1.4 |
| Breast ca. MDA-MB-231 | 4.0 | 1.7 | 1.6 |
| Breast ca. BT 549 | 2.0 | 1.8 | 1.8 |
| Breast ca. T47D | 48.3 | 47.0 | 100.0 |
| Breast ca. MDA-N | 0.0 | 0.0 | 0.0 |
| Breast Pool | 0.3 | 0.3 | 1.1 |
| Trachea | 6.8 | 4.2 | 17.8 |
| Lung | 0.0 | 0.1 | 0.0 |
| Fetal Lung | 12.6 | 10.3 | 31.4 |
| Lung ca. NCI-N417 | 0.7 | 0.3 | 0.3 |
| Lung ca. LX-1 | 0.2 | 0.3 | 12.9 |
| Lung ca. NCI-H146 | 0.0 | 0.0 | 0.2 |
| Lung ca. SHP-77 | 0.0 | 0.0 | 0.0 |
| Lung ca. A549 | 5.1 | 4.8 | 3.6 |
| Lung ca. NCI-H526 | 0.0 | 0.1 | 1.2 |
| Lung ca. NCI-H23 | 6.7 | 6.8 | 4.2 |
| Lung ca. NCI-H460 | 0.0 | 0.1 | 0.1 |
| Lung ca. HOP-62 | 5.3 | 4.6 | 2.1 |
| Lung ca. NCI-H522 | 6.0 | 3.0 | 2.5 |
| Liver | 1.5 | 1.3 | 3.4 |
| Fetal Liver | 4.0 | 4.4 | 22.8 |
| Liver ca. HepG2 | 74.2 | 70.2 | 61.1 |
| Kidney Pool | 0.3 | 0.8 | 0.7 |
| Fetal Kidney | 20.6 | 10.4 | 31.0 |
| Renal ca. 786-0 | 31.9 | 20.4 | 14.9 |
| Renal ca. A498 | 5.6 | 1.4 | 2.2 |
| Renal ca. ACHN | 5.8 | 3.0 | 2.0 |
| Renal ca. UO-31 | 0.9 | 0.8 | 0.9 |
| Renal ca. TK-10 | 31.9 | 32.8 | 27.7 |
| Bladder | 4.0 | 3.9 | 28.1 |
| Gastric ca. (liver met.) NCI-N87 | 0.0 | 0.0 | 2.2 |
| Gastric ca. KATO III | 0.0 | 0.0 | 0.0 |
| Colon ca. SW-948 | 0.1 | 0.0 | 4.5 |
| Colon ca. SW480 | 0.0 | 0.1 | 2.8 |
| Colon ca.* (SW480 met) | 0.7 | 0.3 | 2.3 |
| SW620 | | | |
| Colon ca. HT29 | 0.3 | 0.1 | 17.6 |
| Colon ca. HCT-116 | 0.4 | 0.6 | 21.6 |
| Colon ca. CaCo-2 | 4.2 | 4.7 | 77.4 |
| Colon cancer tissue | 0.1 | 0.0 | 2.2 |
| Colon ca. SW1116 | 0.1 | 0.0 | 4.5 |
| Colon ca. Colo-205 | 0.0 | 0.1 | 3.0 |
| Colon ca. SW-48 | 0.0 | 0.0 | 1.9 |
| Colon Pool | 0.9 | 0.4 | 0.7 |
| Small Intestine Pool | 0.0 | 0.1 | 2.1 |
| Stomach Pool | 0.6 | 0.2 | 1.1 |
| Bone Marrow Pool | 0.1 | 0.0 | 1.0 |
| Fetal Heart | 1.0 | 0.3 | 0.7 |
| Heart Pool | 0.1 | 0.4 | 0.8 |
| Lymph Node Pool | 0.7 | 0.1 | 1.0 |
| Fetal Skeletal Muscle | 0.4 | 0.1 | 0.3 |
| Skeletal Muscle Pool | 0.7 | 0.4 | 0.8 |
| Spleen Pool | 3.5 | 1.3 | 2.0 |
| Thymus Pool | 1.0 | 0.3 | 3.3 |
| CNS cancer (glio/astro) | 0.0 | 0.0 | 0.0 |
| U87-MG | | | |
| CNS cancer (glio/astro) U- | 0.0 | 0.0 | 0.1 |
| 118-MG | | | |
| CNS cancer (neuro; met) | 0.4 | 0.1 | 0.1 |
| SK-N-AS | | | |
| CNS cancer (astro) SF-539 | 2.0 | 0.0 | 1.4 |
| CNS cancer (astro) SNB-75 | 15.3 | 10.2 | 8.9 |
| CNS cancer (glio) SNB-19 | 30.8 | 29.5 | 17.1 |
| CNS cancer (glio) SF-295 | 0.9 | 0.8 | 0.8 |
| Brain (Amygdala) Pool | 51.8 | 32.5 | 47.6 |
| Brain (cerebellum) | 68.8 | 47.6 | 60.7 |
| Brain (fetal) | 10.4 | 9.3 | 12.8 |
| Brain (Hippocampus) Pool | 79.0 | 39.8 | 46.7 |
| Cerebral Cortex Pool | 60.7 | 55.5 | 66.0 |
| Brain (Substantia nigra) | 90.8 | 100.0 | 78.5 |
| Pool | | | |
| Brain (Thalamus) Pool | 59.5 | 55.9 | 64.6 |
| Brain (whole) | 43.8 | 26.8 | 24.7 |
| Spinal Cord Pool | 100.0 | 57.8 | 95.3 |
| Adrenal Gland | 1.1 | 0.5 | 0.3 |
| Pituitary gland Pool | 0.7 | 0.6 | 4.9 |
| Salivary Gland | 0.3 | 0.0 | 0.5 |
| Thyroid (female) | 0.4 | 0.2 | 1.1 |
| Pancreatic ca. CAPAN2 | 0.0 | 0.0 | 0.6 |
| Pancreas Pool | 1.5 | 1.2 | 10.9 |

Column A - Rel. Exp. (%) Ag4039, Run 218425990
Column B - Rel. Exp. (%) Ag4040, Run 218426022
Column C - Rel. Exp. (%) Ag4045, Run 218426255

TABLE LH
Oncology_cell_line_screening_panel_v3.2

| Tissue Name | A | B | C |
|---|---|---|---|
| 94905_Daoy_Medulloblas- | 0.0 | 0.7 | 1.6 |
| toma/Cerebellum_sscDNA | | | |
| 94906_TE671_Medullob- | 0.7 | 2.3 | 1.0 |
| lastom/Cerebellum_sscDNA | | | |
| 94907_D283 | 7.4 | 4.6 | 0.3 |
| Med_Medulloblastoma/ | | | |
| Cerebellum_sscDNA | | | |
| 94908_PFSK- | 75.3 | 82.4 | 31.9 |
| 1_Primitive | | | |
| Neuroectodermal/ | | | |
| Cerebellum_sscDNA | | | |
| 94909_XF- | 27.0 | 24.7 | 9.9 |
| 498_CNS_sscDNA | | | |
| 94910_SNB- | 0.0 | 0.0 | 0.0 |
| 78_CNS/glioma_sscDNA | | | |
| 94911_SF- | 0.0 | 0.0 | 0.4 |
| 268_CNS/glioblastoma_{—} | | | |
| sscDNA | | | |
| 94912_T98G_Glioblasto- | 3.5 | 3.1 | 4.4 |
| ma_sscDNA | | | |
| 96776_SK-N- | 0.9 | 1.5 | 0.4 |
| SH_Neuroblastoma | | | |
| (metastasis)_sscDNA | | | |
| 94913_SF- | 2.8 | 1.6 | 0.9 |
| 295_CNS/glioblastoma_{—} | | | |
| sscDNA | | | |
| 132565_NT2 | 90.1 | 76.3 | 52.1 |
| pool_sscDNA | | | |
| 94914_Cerebellum_sscDNA | 100.0 | 97.9 | 25.2 |
| 96777_Cerebellum_sscDNA | 100.0 | 100.0 | 6.5 |
| 94916_NCI- | 0.0 | 0.0 | 6.7 |
| H292_Mucoepidermoid | | | |
| lung carcinoma_sscDNA | | | |
| 94917_DMS-114_Small | 10.5 | 9.1 | 6.4 |
| cell lung cancer_sscDNA | | | |
| 94918_DMS-79_Small | 4.4 | 1.6 | 33.4 |
| cell lung | | | |
| cancer/neuroendocrine_{—} | | | |
| sscDNA | | | |
| 94919_NCI-H146_Small | 0.0 | 0.0 | 1.1 |
| cell lung | | | |
| cancer/neuroendocrine_{—} | | | |
| sscDNA | | | |
| 94920_NCI-H526_Small | 0.9 | 2.0 | 8.6 |
| cell lung | | | |
| cancer/neuroendocrine_{—} | | | |
| sscDNA | | | |
| 94921_NCI-N417_Small | 0.7 | 0.4 | 0.5 |
| cell lung | | | |
| cancer/neuroendocrine_{—} | | | |
| sscDNA | | | |
| 94923_NCI-H82_Small | 5.8 | 1.3 | 2.6 |
| cell lung | | | |
| cancer/neuroendocrine_{—} | | | |
| sscDNA | | | |
| 94924_NCI- | 2.1 | 1.6 | 0.6 |
| H157_Squamous cell | | | |
| lung cancer | | | |
| (metastasis)_sscDNA | | | |
| 94925_NCI- | 0.0 | 1.3 | 1.8 |
| H1155_Large cell lung | | | |
| cancer/neuroendocrine_{—} | | | |
| sscDNA | | | |
| 94926_NCI- | 5.4 | 4.9 | 3.5 |
| H1299_Large cell lung | | | |
| cancer/neuroendocrine_{—} | | | |
| sscDNA | | | |
| 94927_NCI-H727_Lung | 0.2 | 0.0 | 4.8 |
| carcinoid_sscDNA | | | |
| 94928_NCI-UMC- | 0.0 | 0.0 | 0.2 |
| 11_Lung | | | |
| carcinoid_sscDNA | | | |
| 94929_LX-1_Small cell | 0.0 | 0.0 | 18.7 |
| lung cancer_sscDNA | | | |
| 94930_Colo-205_Colon | 0.0 | 0.0 | 14.8 |
| cancer_sscDNA | | | |
| 94931_KM12_Colon | 0.0 | 0.0 | 1.2 |
| cancer_sscDNA | | | |
| 94932_KM20L2_Colon | 0.0 | 0.0 | 11.2 |
| cancer_sscDNA | | | |
| 94933_NCI-H716_Colon | 18.0 | 20.9 | 0.0 |
| cancer_sscDNA | | | |
| 94935_SW-48_Colon | 0.0 | 0.8 | 6.2 |
| adenocarcinoma_{—} | | | |
| sscDNA | | | |
| 94936_SW1116_Colon | 0.0 | 0.0 | 18.6 |
| adenocarcinoma_{—} | | | |
| sscDNA | | | |
| 94937_LS 174T_Colon | 0.7 | 0.6 | 30.4 |
| adenocarcinoma_{—} | | | |
| sscDNA | | | |
| 94938_SW-948_Colon | 0.0 | 0.0 | 2.1 |
| adenocarcinoma_{—} | | | |
| sscDNA | | | |
| 94939_SW-480_Colon | 0.0 | 0.0 | 8.5 |
| adenocarcinoma_{—} | | | |
| sscDNA | | | |
| 94940_NCI-SNU- | 0.0 | 0.0 | 3.5 |
| 5_Gastric | | | |
| carcinoma_sscDNA | | | |
| 112197_KATO | 0.0 | 0.0 | 0.0 |
| III_Stomach_sscDNA | | | |
| 94943_NCI-SNU- | 0.0 | 0.0 | 0.0 |
| 16_Gastric | | | |
| carcinoma_sscDNA | | | |
| 94944_NCI-SNU- | 0.0 | 0.0 | 0.0 |
| 1_Gastric | | | |
| carcinoma_sscDNA | | | |
| 94946_RF-1_Gastric | 80.1 | 0.0 | 0.0 |
| adenocarcinoma_{—} | | | |
| sscDNA | | | |
| 94947_RF-48_Gastric | 0.0 | 0.0 | 0.0 |
| adenocarcinoma_sscDNA | | | |
| 96778_MKN-45_Gastric | 0.0 | 0.3 | 23.5 |
| carcinoma_sscDNA | | | |
| 94949_NCI-N87_Gastric | 0.2 | 0.0 | 6.8 |
| carcinoma_sscDNA | | | |
| 94951_OVCAR- | 0.0 | 0.0 | 12.1 |
| 5_Ovarian | | | |
| carcinoma_sscDNA | | | |
| 94952_RL95-2_Uterine | 0.8 | 0.0 | 19.1 |
| carcinoma_sscDNA | | | |
| 94953_HelaS3_Cervical | 3.6 | 1.0 | 7.2 |
| adenocarcinoma_{—} | | | |
| sscDNA | | | |
| 94954_Ca Ski_Cervical | 0.8 | 0.0 | 2.3 |
| epidermoid carcinoma | | | |
| (metastasis)_sscDNA | | | |
| 94955_ES-2_Ovarian clear | 0.0 | 0.0 | 0.0 |
| cell carcinoma_sscDNA | | | |
| 94957_Ramos/6h stim_{—} | 0.0 | 0.0 | 0.0 |
| Stimulated with | | | |
| PMA/ionomycin | | | |
| 6h_sscDNA | | | |
| 94958_Ramos/14h stim_{—} | 0.0 | 0.0 | 0.0 |
| Stimulated with | | | |
| PMA/ionomycin | | | |
| 14h_sscDNA | | | |
| 94962_MEG-01_Chronic | 5.8 | 3.8 | 3.6 |
| myelogenous leukemia | | | |
| (megokaryoblast)_sscDNA | | | |
| 94963_Raji_Burkitt's | 0.0 | 0.0 | 0.0 |
| lymphoma_sscDNA | | | |
| 94964_Daudi_Burkitt's | 0.0 | 0.0 | 0.0 |
| lymphoma_sscDNA | | | |
| 94965_U266_B-cell | 0.0 | 0.4 | 0.3 |
| plasmacytoma/myeloma_{—} | | | |
| sscDNA | | | |
| 94968_CA46_Burkitt's | 0.0 | 0.0 | 0.0 |
| lymphoma_sscDNA | | | |
| 94970_RL_non-Hodgkin's | 0.0 | 0.0 | 0.0 |
| B-cell lymphoma_sscDNA | | | |
| 94972_JM1_pre-B-cell | 0.0 | 0.0 | 1.0 |
| lymphoma/leukemia_sscDNA | | | |
| 94973_Jurkat_T cell | 5.5 | 6.3 | 2.0 |
| leukemia_sscDNA | | | |
| 94974_TF- | 2.6 | 9.7 | 4.5 |
| 1_Erythroleukemia_sscDNA | | | |
| 94975_HUT 78_T-cell | 6.0 | 2.9 | 4.8 |
| lymphoma_sscDNA | | | |
| 94977_U937_Histiocytic | 2.4 | 1.0 | 0.0 |
| lymphoma_sscDNA | | | |
| 94980_KU- | 19.6 | 18.2 | 7.5 |
| 812_Myelogenous | | | |
| leukemia_sscDNA | | | |
| 94981_769-P_Clear cell | 57.0 | 61.1 | 50.3 |
| renal carcinoma_sscDNA | | | |
| 94983_Caki-2_Clear cell | 0.8 | 4.3 | 4.0 |
| renal carcinoma_sscDNA | | | |
| 94984_SW 839_Clear cell | 0.0 | 0.0 | 0.0 |
| renal carcinoma_sscDNA | | | |
| 94986_G401_Wilms' | 10.2 | 6.8 | 3.5 |
| tumor_sscDNA | | | |
| 126768_293 cells_sscDNA | 15.8 | 23.8 | 18.6 |
| 94987_Hs766T_Pancreatic | 0.6 | 1.0 | 2.1 |
| carcinoma (LN | | | |
| metastasis)_sscDNA | | | |
| 94988_CAPAN- | 1.9 | 0.0 | 0.8 |
| 1_Pancreatic | | | |
| adenocarcinoma (liver | | | |
| metastasis)_sscDNA | | | |
| 94989_SU86.86_Pancreatic | 0.0 | 0.0 | 6.1 |
| carcinoma (liver | | | |
| metastasis)_sscDNA | | | |
| 94990_BxPC-3_Pancreatic | 0.8 | 0.4 | 100.0 |
| adenocarcinoma_sscDNA | | | |
| 94991_HPAC_Pancreatic | 0.0 | 0.0 | 1.2 |
| adenocarcinoma_sscDNA | | | |
| 94992_MIA PaCa- | 0.7 | 0.8 | 3.1 |
| 2_Pancreatic | | | |
| carcinoma_sscDNA | | | |
| 94993_CFPAC- | 0.7 | 1.8 | 24.0 |
| 1_Pancreatic ductal | | | |
| adenocarcinoma_sscDNA | | | |
| 94994_PANC-1_Pancreatic | 5.8 | 5.3 | 11.8 |
| epithelioid ductal | | | |
| carcinoma_sscDNA | | | |
| 94996_T24_Bladder | 0.0 | 0.0 | 0.0 |
| carcinma (transitional | | | |
| cell)_sscDNA | | | |
| 94997_5637_Bladder | 0.0 | 0.0 | 0.8 |
| carcinoma_sscDNA | | | |
| 94998_HT-1197_Bladder | 0.0 | 0.0 | 10.2 |
| carcinoma_sscDNA | | | |
| 94999_UM-UC-3_Bladder | 0.6 | 5.5 | 1.7 |
| carcinma (transitional | | | |
| cell)_sscDNA | | | |
| 95000_A204_Rhabdomyos- | 8.3 | 20.9 | 10.4 |
| arcoma_sscDNA | | | |
| 95001_HT- | 2.3 | 4.0 | 3.0 |
| 1080_Fibrosarcoma_{—} | | | |
| sscDNA | | | |
| 95002_MG- | 0.0 | 0.9 | 0.7 |
| 63_Osteosarcoma | | | |
| (bone)_sscDNA | | | |
| 95003_SK-LMS- | 0.0 | 0.0 | 0.0 |
| 1_Leiomyosarcoma | | | |
| (vulva)_sscDNA | | | |
| 95004_SJRH30_Rhabdomy- | 1.4 | 0.0 | 4.5 |
| osarcoma (met to bone | | | |
| marrow)_sscDNA | | | |
| 95005_A431_Epidermoid | 0.0 | 0.0 | 18.6 |
| carcinoma_sscDNA | | | |
| 95007_WM266- | 0.0 | 0.0 | 0.0 |
| 4_Melanoma_sscDNA | | | |
| 112195_DU | 0.7 | 0.0 | 3.6 |
| 145_Prostate_sscDNA | | | |
| 95012_MDA-MB- | 0.0 | 0.0 | 11.5 |
| 468_Breast | | | |
| adenocarcinoma_sscDNA | | | |
| 112196_SSC- | 1.5 | 0.0 | 39.5 |
| 4_Tongue_sscDNA | | | |
| 112194_SSC- | 0.7 | 0.0 | 11.5 |
| 9_Tongue_sscDNA | | | |
| 112191_SSC- | 0.0 | 0.0 | 26.2 |
| 15_Tongue_sscDNA | | | |
| 95017_CAL 27_Squamous | 0.0 | 0.5 | 54.0 |
| cell carcinoma of | | | |
| tongue_sscDNA | | | |

Column A - Rel. Exp. (%) Ag4039, Run 257342469
Column B - Rel. Exp. (%) Ag4040, Run 257342474
Column C - Rel. Exp. (%) Ag4045, Run 257342527

TABLE LI
Panel 4.1D

| Tissue Name | A | B | C |
|---|---|---|---|
| Secondary Th1 act | 0.0 | 0.0 | 0.0 |
| Secondary Th2 act | 0.0 | 0.0 | 0.0 |
| Secondary Tr1 act | 6.2 | 0.0 | 0.4 |
| Secondary Th1 rest | 2.4 | 0.0 | 0.0 |
| Secondary Th2 rest | 0.0 | 0.0 | 0.0 |
| Secondary Tr1 rest | 0.0 | 2.6 | 0.0 |
| Primary Th1 act | 0.0 | 0.0 | 0.0 |
| Primary Th2 act | 0.0 | 0.0 | 0.5 |
| Primary Tr1 act | 0.0 | 0.0 | 0.0 |
| Primary Th1 rest | 0.0 | 0.0 | 0.0 |
| Primary Th2 rest | 0.0 | 0.0 | 0.0 |
| Primary Tr1 rest | 2.4 | 3.5 | 0.0 |
| CD45RA CD4 | 0.0 | 6.5 | 0.9 |
| lymphocyte act | | | |
| CD45RO CD4 | 0.0 | 0.0 | 0.0 |
| lymphocyte act | | | |
| CD8 lymphocyte act | 0.0 | 0.0 | 0.0 |
| Secondary CD8 | 0.0 | 0.0 | 0.0 |
| lymphocyte rest | | | |
| Secondary CD8 | 0.0 | 0.0 | 0.4 |
| lymphocyte act | | | |
| CD4 lymphocyte none | 0.0 | 0.0 | 0.0 |
| 2ry Th1/Th2/Tr1_anti- | 2.3 | 0.0 | 0.0 |
| CD95 CH11 | | | |
| LAK cells rest | 2.4 | 0.0 | 0.0 |
| LAK cells IL-2 | 0.0 | 0.0 | 0.0 |
| LAK cells IL-2 + IL-12 | 0.0 | 0.0 | 0.0 |
| LAK cells IL-2 + IFN | 0.0 | 0.0 | 0.6 |
| gamma | | | |
| LAK cells IL-2 + IL-18 | 0.0 | 0.0 | 0.0 |
| LAK cells | 2.9 | 0.0 | 0.4 |
| PMA/ionomycin | | | |
| NK Cells IL-2 rest | 0.0 | 0.0 | 0.0 |
| Two Way MLR 3 day | 0.0 | 0.0 | 0.0 |
| Two Way MLR 5 day | 0.0 | 0.0 | 0.0 |
| Two Way MLR 7 day | 0.0 | 0.0 | 0.0 |
| PBMC rest | 4.2 | 0.0 | 0.0 |
| PBMC PWM | 0.0 | 0.0 | 0.0 |
| PBMC PHA-L | 0.0 | 0.0 | 0.0 |
| Ramos (B cell) none | 0.0 | 0.0 | 0.0 |
| Ramos (B cell) | 0.0 | 0.0 | 0.0 |
| ionomycin | | | |
| B lymphocytes PWM | 0.0 | 0.0 | 0.0 |
| B lymphocytes CD40L | 0.0 | 0.0 | 0.1 |
| and IL-4 | | | |
| EOL-1 dbcAMP | 14.9 | 16.3 | 9.3 |
| EOL-1 dbcAMP | 15.9 | 14.0 | 6.2 |
| PMA/ionomycin | | | |
| Dendritic cells none | 0.0 | 0.0 | 0.0 |
| Dendritic cells LPS | 0.0 | 0.0 | 0.0 |
| Dendritic cells anti-CD40 | 0.0 | 0.0 | 0.0 |
| Monocytes rest | 0.0 | 0.0 | 0.0 |
| Monocytes LPS | 0.0 | 0.0 | 0.0 |
| Macrophages rest | 0.0 | 0.0 | 0.0 |
| Macrophages LPS | 0.0 | 0.0 | 0.0 |
| HUVEC none | 0.0 | 0.0 | 0.0 |
| HUVEC starved | 8.4 | 2.3 | 2.0 |
| HUVEC IL-1beta | 1.8 | 1.9 | 0.6 |
| HUVEC IFN gamma | 3.4 | 4.7 | 0.4 |
| HUVEC TNF alpha + IFN | 1.6 | 0.0 | 0.5 |
| gamma | | | |
| HUVEC TNF alpha + IL4 | 0.0 | 0.5 | 1.1 |
| HUVEC IL-11 | 1.6 | 3.1 | 1.2 |
| Lung Microvascular EC none | 25.7 | 36.9 | 7.6 |
| Lung Microvascular EC | 5.4 | 13.0 | 4.4 |
| TNFalpha + IL-1beta | | | |
| Microvascular Dermal EC | 1.4 | 2.0 | 0.0 |
| none | | | |
| Microsvasular Dermal EC | 0.0 | 1.9 | 1.7 |
| TNFalpha + IL-1beta | | | |
| Bronchial epithelium | 4.3 | 5.6 | 27.4 |
| TNFalpha + IL1beta | | | |
| Small airway epithelium | 1.6 | 0.0 | 35.4 |
| none | | | |
| Small airway epithelium | 4.3 | 0.9 | 94.0 |
| TNFalpha + IL-1beta | | | |
| Coronery artery SMC rest | 0.0 | 4.0 | 0.0 |
| Coronery artery SMC | 2.0 | 0.0 | 0.6 |
| TNFalpha + IL-1beta | | | |
| Astrocytes rest | 27.9 | 49.3 | 3.6 |
| Astrocytes TNFalpha + IL- | 14.5 | 34.2 | 2.9 |
| 1beta | | | |
| KU-812 (Basophil) rest | 24.1 | 35.1 | 8.2 |
| KU-812 (Basophil) | 5.1 | 9.4 | 2.7 |
| PMA/ionomycin | | | |
| CCD1106 (Keratinocytes) | 1.9 | 0.0 | 15.8 |
| none | | | |
| CCD1106 (Keratinocytes) | 1.8 | 4.4 | 16.5 |
| TNFalpha + IL-1beta | | | |
| Liver cirrhosis | 3.3 | 7.8 | 25.2 |
| NCI-H292 none | 0.0 | 2.7 | 5.9 |
| NCI-H292 IL-4 | 0.0 | 0.0 | 7.4 |
| NCI-H292 IL-9 | 0.0 | 0.0 | 2.9 |
| NCI-H292 IL-13 | 0.0 | 0.0 | 5.6 |
| NCI-H292 IFN gamma | 6.1 | 0.0 | 2.3 |
| HPAEC none | 0.0 | 5.1 | 1.8 |
| HPAEC TNF alpha + IL-1 | 0.0 | 2.1 | 0.0 |
| beta | | | |
| Lung fibroblast none | 0.0 | 0.0 | 0.2 |
| Lung fibroblast TNF alpha + | 0.0 | 7.8 | 0.0 |
| IL-1 beta | | | |
| Lung fibroblast IL-4 | 1.6 | 0.0 | 0.5 |
| Lung fibroblast IL-9 | 0.0 | 0.0 | 0.0 |
| Lung fibroblast IL-13 | 0.0 | 0.0 | 0.0 |
| Lung fibroblast IFN gamma | 6.0 | 2.4 | 1.3 |
| Dermal fibroblast CCD1070 | 3.3 | 18.3 | 2.4 |
| rest | | | |
| Dermal fibroblast CCD1070 | 15.2 | 11.4 | 2.8 |
| TNF alpha | | | |
| Dermal fibroblast CCD1070 | 1.6 | 3.2 | 2.3 |
| IL-1 beta | | | |
| Dermal fibroblast IFN | 11.3 | 3.4 | 1.0 |
| gamma | | | |
| Dermal fibroblast IL-4 | 8.2 | 0.0 | 1.0 |
| Dermal Fibroblasts rest | 0.0 | 3.0 | 0.3 |
| Neutrophils TNFa + LPS | 0.0 | 0.0 | 0.4 |
| Neutrophils rest | 0.0 | 0.0 | 0.7 |
| Colon | 2.8 | 2.9 | 11.1 |
| Lung | 0.0 | 0.0 | 4.2 |
| Thymus | 22.8 | 8.2 | 8.7 |
| Kidney | 100.0 | 100.0 | 100.0 |

Column A - Rel. Exp. (%) Ag4039, Run 171616916
Column B - Rel. Exp. (%) Ag4040, Run 171616917
Column C - Rel. Exp. (%) Ag4045, Run 171617030

TABLE LJ
Panel 5D

| Tissue Name | A | B | C |
|---|---|---|---|
| 97457_Patient- | 0.0 | 2.0 | 0.0 |
| 02go_adipose | | | |
| 97476_Patient- | 1.4 | 0.0 | 0.7 |
| 07sk_skeletal muscle | | | |
| 97477_Patient- | 0.0 | 1.4 | 0.2 |
| 07ut_uterus | | | |
| 97478_Patient- | 8.7 | 12.7 | 4.4 |
| 07pl_placenta | | | |
| 97481_Patient- | 0.0 | 0.0 | 0.3 |
| 08sk_skeletal muscle | | | |
| 97482_Patient- | 0.0 | 0.0 | 0.0 |
| 08ut_uterus | | | |
| 97483_Patient- | 5.8 | 12.2 | 4.9 |
| 08pl_placenta | | | |
| 97486_Patient- | 0.0 | 0.0 | 0.0 |
| 09sk_skeletal muscle | | | |
| 97487_Patient- | 0.0 | 2.3 | 0.0 |
| 09ut_uterus | | | |
| 97488_Patient- | 13.5 | 7.7 | 6.9 |
| 09pl_placenta | | | |
| 97492_Patient- | 0.0 | 2.1 | 0.0 |
| 10ut_uterus | | | |
| 97493_Patient- | 22.1 | 14.6 | 8.5 |
| 10pl_placenta | | | |
| 97495_Patient- | 1.6 | 0.0 | 0.8 |
| 11go_adipose | | | |
| 97496_Patient- | 0.0 | 1.3 | 0.3 |
| 11sk_skeletal muscle | | | |
| 97497_Patient- | 0.0 | 0.0 | 0.0 |
| 11ut_uterus | | | |
| 97498_Patient- | 14.0 | 15.7 | 5.0 |
| 11pl_placenta | | | |
| 97500_Patient- | 1.8 | 1.5 | 1.7 |
| 12go_adipose | | | |
| 97501_Patient- | 1.5 | 1.1 | 0.5 |
| 12sk_skeletal muscle | | | |
| 97502_Patient- | 1.6 | 1.7 | 1.1 |
| 12ut_uterus | | | |
| 97503_Patient- | 11.3 | 11.3 | 2.7 |
| 12pl_placenta | | | |
| 94721_Donor 2 U - | 0.0 | 0.0 | 0.9 |
| A_Mesenchymal | | | |
| Stem Cells | | | |
| 94722_Donor 2 U - | 0.0 | 0.0 | 0.0 |
| B_Mesenchymal | | | |
| Stem Cells | | | |
| 94723_Donor 2 U - | 0.0 | 2.1 | 1.2 |
| C_Mesenchymal | | | |
| Stem Cells | | | |
| 94709_Donor 2 AM - A_adipose | 1.0 | 9.0 | 0.3 |
| 94710_Donor 2 AM - B_adipose | 0.9 | 0.0 | 0.9 |
| 94711_Donor 2 AM - C_adipose | 0.0 | 2.4 | 0.4 |
| 94712_Donor 2 AD - A_adipose | 0.0 | 0.0 | 0.0 |
| 94713_Donor 2 AD - B_adipose | 0.0 | 0.0 | 0.0 |
| 94714_Donor 2 AD - C_adipose | 1.4 | 0.0 | 0.3 |
| 94742_Donor 3 U - | 0.0 | 0.0 | 1.2 |
| A_Mesenchymal Stem Cells | | | |
| 94743_Donor 3 U - | 2.7 | 0.0 | 0.4 |
| B_Mesenchymal Stem Cells | | | |
| 94730_Donor 3 AM - A_adipose | 1.6 | 6.2 | 1.2 |
| 94731_Donor 3 AM - B_adipose | 3.8 | 4.1 | 0.4 |
| 94732_Donor 3 AM - C_adipose | 1.3 | 2.3 | 0.8 |
| 94733_Donor 3 AD - A_adipose | 6.7 | 0.0 | 1.7 |
| 94734_Donor 3 AD - B_adipose | 3.1 | 0.0 | 0.7 |
| 94735_Donor 3 AD - C_adipose | 0.2 | 0.9 | 1.7 |
| 77138_Liver_HepG2untreated | 100.0 | 100.0 | 100.0 |
| 73556_Heart_Cardiac stromal | 0.0 | 0.0 | 0.0 |
| cells (primary) | | | |
| 81735_Small Intestine | 0.0 | 0.0 | 8.1 |
| 72409_Kidney_Proximal | 0.0 | 0.0 | 0.0 |
| Convoluted Tubule | | | |
| 82685_Small | 2.0 | 2.0 | 7.0 |
| intestine_Duodenum | | | |
| 90650_Adrenal_Adrenocortical | 0.0 | 0.0 | 0.3 |
| adenoma | | | |
| 72410_Kidney_HRCE | 0.0 | 1.9 | 0.4 |
| 72411_Kidney_HRE | 2.6 | 0.0 | 1.7 |
| 73139_Uterus_Uterine smooth | 0.0 | 2.2 | 0.2 |
| muscle cells | | | |

Column A - Rel. Exp. (%) Ag4039, Run 257460989
Column B - Rel. Exp. (%) Ag4040, Run 257460990
Column C - Rel. Exp. (%) Ag4045, Run 257488184

TABLE LK
general oncology screening panel_v_2.4

| Tissue Name | A | B | C |
|---|---|---|---|
| Colon cancer 1 | 2.3 | 1.0 | 6.8 |
| Colon NAT 1 | 0.5 | 1.3 | 6.2 |
| Colon cancer 2 | 2.6 | 1.9 | 0.3 |
| Colon NAT 2 | 0.5 | 0.7 | 7.7 |
| Colon cancer 3 | 1.3 | 3.8 | 9.7 |
| Colon NAT 3 | 3.6 | 0.8 | 12.4 |
| Colon malignant cancer 4 | 0.5 | 0.0 | 8.4 |
| Colon NAT 4 | 1.3 | 1.0 | 1.3 |
| Lung cancer 1 | 1.3 | 0.0 | 2.2 |
| Lung NAT 1 | 0.0 | 0.9 | 0.4 |
| Lung cancer 2 | 100.0 | 100.0 | 100.0 |
| Lung NAT 2 | 2.7 | 1.9 | 1.2 |
| Squamous cell carcinoma 3 | 0.0 | 1.3 | 33.2 |
| Lung NAT 3 | 0.0 | 0.0 | 0.5 |
| Metastatic melanoma 1 | 0.0 | 1.0 | 0.3 |
| Melanoma 2 | 1.0 | 0.0 | 22.4 |
| Melanoma 3 | 0.0 | 0.0 | 2.5 |
| Metastatic melanoma 4 | 1.9 | 1.2 | 0.3 |
| Metastatic melanoma 5 | 3.7 | 3.4 | 0.1 |
| Bladder cancer 1 | 0.0 | 0.0 | 0.0 |
| Bladder NAT 1 | 0.0 | 0.0 | 0.0 |
| Bladder cancer 2 | 0.0 | 0.0 | 2.7 |
| Bladder NAT 2 | 0.0 | 0.0 | 0.0 |
| Bladder NAT 3 | 0.0 | 0.0 | 1.3 |
| Bladder NAT 4 | 0.2 | 0.7 | 0.4 |
| Prostate adenocarcinoma 1 | 0.0 | 0.0 | 2.0 |
| Prostate adenocarcinoma 2 | 1.0 | 2.0 | 1.4 |
| Prostate adenocarcinoma 3 | 4.3 | 0.8 | 5.2 |
| Prostate adenocarcinoma 4 | 0.7 | 0.0 | 7.6 |
| Prostate NAT 5 | 0.0 | 0.0 | 6.0 |
| Prostate adenocarcinoma 6 | 0.9 | 1.4 | 2.1 |
| Prostate adenocarcinoma 7 | 1.3 | 1.2 | 0.7 |
| Prostate adenocarcinoma 8 | 1.1 | 0.0 | 0.3 |
| Prostate adenocarcinoma 9 | 0.8 | 1.3 | 0.7 |
| Prostate NAT 10 | 0.8 | 1.3 | 1.1 |
| Kidney cancer 1 | 13.9 | 11.6 | 8.7 |
| Kidney NAT 1 | 3.5 | 6.8 | 18.4 |
| Kidney cancer 2 | 66.4 | 66.9 | 32.3 |
| Kidney NAT 2 | 60.3 | 28.5 | 76.3 |
| Kidney cancer 3 | 25.2 | 42.3 | 49.0 |
| Kidney NAT 3 | 21.5 | 13.2 | 91.4 |
| Kidney cancer 4 | 72.2 | 43.5 | 15.7 |
| Kidney NAT 4 | 48.6 | 53.6 | 18.7 |

Column A - Rel. Exp. (%) Ag4039, Run 268362924
Column B - Rel. Exp. (%) Ag4040, Run 268362927
Column C - Rel. Exp. (%) Ag4045, Run 268362935

AI.05 chondrosarcoma Summary: Ag4039/Ag4045 Highest expression of this gene is detected in untreated serum starved chondrosarcoma cell line (SW1353) (CTs29-32). Interestingly, expression of this gene appears to be somewhat down regulated upon IL-1 treatment, a potent activator of pro-inflammatory cytokines and matrix metalloproteinases which participate in the destruction of cartilage observed in Osteoarthritis (OA). Therefore, therapeutic modulation of the activity of this gene or its protein product may be important for preventing the degeneration of cartilage observed in osteoarthritis.

AI_comprehensive panel_v1.0 Summary: Ag4039/Ag4040/Ag4045 Three experiments with two different probe-primer sets are in good agreement with highest expression of this gene seen in osteoarthritis (OA) cartilage samples (CTs=29). Significant expression of this gene is mainly detected in samples derived from OA bone and adjacent normal bone, and OA cartilage. Therefore, therapeutic modulation of this gene or its protein product may be useful in the treatment of osteoarthritis.

CNS_neurodegeneration_v1.0 Summary: Data obtained from RTQPCR assays performed for this panel was analyized using multivariate analysis (ANOVA). The multivariate results obtained analyzing Ag4039/Ag4040/Ag4045 confirms the expression of this gene at low levels in the brain in an independent group of individuals. This gene is found to be down-regulated in the temporal cortex of Alzheimer's disease patients (p=0.0007). Therefore, up-regulation of this gene or its protein product, or treatment with specific agonists for this receptor may be of use in reversing the dementia/memory loss associated with this disease and neuronal death.

General_screening_panel_v1.4 Summary: Ag4039/Ag4040/Ag4045 Three experiments with two different probe-primer sets are in good agreement with highest expression of this gene seen in spinal cord, brain substantia nigra and a breast cancer T47D cell line (CTs=26-27.4). High expression of this gene is mainly seen in all the regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Oncology_cell_line_screening_panel_v3.2 Summary: Ag4039/Ag4040 Two experiments with same probe-primer sets are in good agreement. Highest expression of this gene seen in cerebellum samples (CTs=31). Significant expression of this gene is seen in number of cell lines derived from brain, colon, gastric, renal and bone marrow cancers.

Ag4045 Highest expression of this gene is seen in pancreatic adenocarcinoma cell line samples (CT=30.7). Moderate to low expression of this gene is seen in several cancer cell lines derived from brain, lung, colon, gastric, ovarian, uterine, pancreatic, renal, bone marrow, bladder, epidermoid, ovarian and tongue cancers. Therefore, therapeutic modulation of this gene or its gene product may be useful in the treatment of these cancers.

Panel 4.1D Summary: Ag4039/Ag4040/Ag4045 Highest expression of this gene is seen in kidney (CT=31.3). Moderate to low expression of this gene is seen in eosinophils, lung microvascular endothelial cells, astrocytes, basophils, and activated dermal fibroblasts. The variant gene recognized by the probe-primer set Ag4045 also shows expression in bronchial and small airway epithelium, mucoepidermoid NCI-H292 cell line, keratinocytes, and normal tissues represented by colon, lung and thymus. Therefore, therapeutic modulation of this gene or its protein product may be useful in treatment of autoimmune and inflammatory diseases such as asthma, allergies, inflammatory bowel disease, lupus erythematosus, psoriasis, rheumatoid arthritis, and osteoarthritis.

Panel 5D Summary: Ag4039/Ag4040/Ag4045 Highest expression of this gene is seen in liver cancer HepG2 cell line (CTs=31.3). Low expression of this gene is also seen in placenta from diabetic and non-diabetic patient.

general oncology screening panel_v_{—}2.4 Summary: Ag4039/Ag4040/Ag4045 Three experiments with two different probe-primer sets are in good agreement with highest expression of this gene seen in lung cancer sample (CTs=28-30.8). Significant expression of this gene is also seen in normal and cancer samples from kidney. The variant gene recognized by the probe-primer set Ag4045 also shows expression in normal and cancer samples derived from prostate, metastatic melanoma, lung and colon.

M. CG57341-01: Short Chain Dehydrogenase/reductase 1

Expression of gene CG57341-01 was assessed using the primer-probe set Ag3204, described in Table MA. Results of the RTQ-PCR runs are shown in Tables MB, MC and MD.

TABLE MA

Probe Name Ag3204

| | | | | |
|---|---|---|---|---|
| | | | Start | SEQ ID |
| Primers | Sequences | Length | Position | No |

| | | | | |
|---|---|---|---|---|
| Forward | 5′-ggactttgatcccctacagatg-3′ | 22 | 170 | 149 |
| Probe | TET-5′-tcaaatgaagaggacatcctctccat-3′-TAMRA | 26 | 199 | 150 |
| Reverse | 5′-ctgagaacggatagctgagaac-3′ | 22 | 225 | 151 |

TABLE MB
CNS_neurodegeneration_v1.0

| Tissue Name | A | B |
|---|---|---|
| AD 1 Hippo | 3.9 | 0.1 |
| AD 2 Hippo | 15.3 | 0.2 |
| AD 3 Hippo | 3.3 | 0.1 |
| AD 4 Hippo | 3.9 | 0.1 |
| AD 5 hippo | 67.4 | 0.9 |
| AD 6 Hippo | 20.9 | 0.3 |
| Control 2 Hippo | 24.7 | 0.4 |
| Control 4 Hippo | 5.4 | 0.1 |
| Control (Path) 3 Hippo | 3.9 | 0.1 |
| AD 1 Temporal Ctx | 7.5 | 0.1 |
| AD 2 Temporal Ctx | 10.5 | 0.3 |
| AD 3 Temporal Ctx | 2.7 | 0.1 |
| AD 4 Temporal Ctx | 12.8 | 0.2 |
| AD 5 Inf Temporal Ctx | 57.0 | 0.0 |
| AD 5 Sup Temporal Ctx | 22.8 | 0.4 |
| AD 6 Inf Temporal Ctx | 26.2 | 0.5 |
| AD 6 Sup Temporal Ctx | 30.6 | 0.4 |
| Control 1 Temporal Ctx | 7.7 | 0.1 |
| Control 2 Temporal Ctx | 48.0 | 0.9 |
| Control 3 Temporal Ctx | 17.1 | 0.2 |
| Control 4 Temporal Ctx | 4.6 | 0.1 |
| Control (Path) 1 Temporal Ctx | 55.1 | 0.8 |
| Control (Path) 2 Temporal Ctx | 39.5 | 0.5 |
| Control (Path) 3 Temporal Ctx | 3.7 | 0.1 |
| Control (Path) 4 Temporal Ctx | 25.5 | 0.3 |
| AD 1 Occipital Ctx | 7.2 | 0.1 |
| AD 2 Occipital Ctx (Missing) | 0.0 | 0.0 |
| AD 3 Occipital Ctx | 3.9 | 0.0 |
| AD 4 Occipital Ctx | 14.7 | 0.2 |
| AD 5 Occipital Ctx | 16.8 | 0.7 |
| AD 6 Occipital Ctx | 64.2 | 0.2 |
| Control 1 Occipital Ctx | 5.1 | 0.1 |
| Control 2 Occipital Ctx | 100.0 | 1.5 |
| Control 3 Occipital Ctx | 18.6 | 0.3 |
| Control 4 Occipital Ctx | 5.3 | 100.0 |
| Control (Path) 1 Occipital Ctx | 78.5 | 1.1 |
| Control (Path) 2 Occipital Ctx | 12.1 | 0.2 |
| Control (Path) 3 Occipital Ctx | 3.3 | 0.1 |
| Control (Path) 4 Occipital Ctx | 22.5 | 0.3 |
| Control 1 Parietal Ctx | 7.1 | 0.1 |
| Control 2 Parietal Ctx | 29.1 | 0.3 |
| Control 3 Parietal Ctx | 23.7 | 0.3 |
| Control (Path) 1 Parietal Ctx | 92.7 | 1.2 |
| Control (Path) 2 Parietal Ctx | 24.5 | 0.5 |
| Control (Path) 3 Parietal Ctx | 5.3 | 0.1 |
| Control (Path) 4 Parietal Ctx | 44.1 | 0.5 |

Column A - Rel. Exp. (%) Ag3204, Run 209861769
Column B - Rel. Exp. (%) Ag3204, Run 249265914

TABLE MC
Panel 1.3D

| Tissue Name | A |
|---|---|
| Liver adenocarcinoma | 23.8 |
| Pancreas | 9.9 |
| Pancreatic ca. CAPAN 2 | 9.6 |
| Adrenal gland | 3.8 |
| Thyroid | 10.0 |
| Salivary gland | 5.9 |
| Pituitary gland | 10.5 |
| Brain (fetal) | 14.0 |
| Brain (whole) | 46.3 |
| Brain (amygdala) | 16.8 |
| Brain (cerebellum) | 22.7 |
| Brain (hippocampus) | 18.9 |
| Brain (substantia nigra) | 20.3 |
| Brain (thalamus) | 26.6 |
| Cerebral Cortex | 66.4 |
| Spinal cord | 6.9 |
| glio/astro U87-MG | 11.8 |
| glio/astro U-118-MG | 5.0 |
| astrocytoma SW1783 | 23.7 |
| neuro*; met SK-N-AS | 12.9 |
| astrocytoma SF-539 | 5.4 |
| astrocytoma SNB-75 | 9.4 |
| glioma SNB-19 | 13.9 |
| glioma U251 | 28.5 |
| glioma SF-295 | 21.9 |
| Heart (fetal) | 21.9 |
| Heart | 9.7 |
| Skeletal muscle (fetal) | 13.3 |
| Skeletal muscle | 18.8 |
| Bone marrow | 5.2 |
| Thymus | 4.7 |
| Spleen | 3.1 |
| Lymph node | 6.5 |
| Colorectal | 69.7 |
| Stomach | 8.7 |
| Small intestine | 77.4 |
| Colon ca. SW480 | 26.4 |
| Colon ca.* SW620 (SW480 met) | 47.3 |
| Colon ca. HT29 | 47.0 |
| Colon ca. HCT-116 | 14.0 |
| Colon ca. CaCo-2 | 100.0 |
| Colon ca. tissue (ODO3866) | 4.9 |
| Colon ca. HCC-2998 | 28.1 |
| Gastric ca.* (liver met) NCI-N87 | 18.6 |
| Bladder | 5.3 |
| Trachea | 1.9 |
| Kidney | 27.2 |
| Kidney (fetal) | 23.2 |
| Renal ca. 786-0 | 7.0 |
| Renal ca. A498 | 11.7 |
| Renal ca. RXF 393 | 8.8 |
| Renal ca. ACHN | 9.7 |
| Renal ca. UO-31 | 2.6 |
| Renal ca. TK-10 | 22.8 |
| Liver | 7.8 |
| Liver (fetal) | 8.2 |
| Liver ca. (hepatoblast) HepG2 | 36.9 |
| Lung | 2.2 |
| Lung (fetal) | 5.6 |
| Lung ca. (small cell) LX-1 | 19.1 |
| Lung ca. (small cell) NCI-H69 | 6.1 |
| Lung ca. (s. cell var.) SHP-77 | 75.3 |
| Lung ca. (large cell) NCI-H460 | 1.5 |
| Lung ca. (non-sm. cell) A549 | 13.4 |
| Lung ca. (non-s. cell) NCI-H23 | 5.0 |
| Lung ca. (non-s. cell) HOP-62 | 10.7 |
| Lung ca. (non-s. cl) NCI-H522 | 46.3 |
| Lung ca. (squam.) SW 900 | 4.8 |
| Lung ca. (squam.) NCI-H596 | 23.5 |
| Mammary gland | 16.5 |
| Breast ca.* (pl. ef) MCF-7 | 1.5 |
| Breast ca.* (pl. ef) MDA-MB-231 | 13.0 |
| Breast ca.* (pl. ef) T47D | 35.6 |
| Breast ca. BT-549 | 5.1 |
| Breast ca. MDA-N | 12.0 |
| Ovary | 8.1 |
| Ovarian ca. OVCAR-3 | 10.2 |
| Ovarian ca. OVCAR-4 | 15.3 |
| Ovarian ca. OVCAR-5 | 46.0 |
| Ovarian ca. OVCAR-8 | 3.5 |
| Ovarian ca. IGROV-1 | 4.4 |
| Ovarian ca.* (ascites) SK-OV-3 | 45.4 |
| Uterus | 2.6 |
| Placenta | 0.6 |
| Prostate | 5.8 |
| Prostate ca.* (bone met) PC-3 | 4.5 |
| Testis | 7.9 |
| Melanoma Hs688(A).T | 0.9 |
| Melanoma* (met) Hs688(B).T | 0.8 |
| Melanoma UACC-62 | 13.0 |
| Melanoma M14 | 3.2 |
| Melanoma LOX IMVI | 18.2 |
| Melanoma* (met) SK-MEL-5 | 15.6 |
| Adipose | 6.6 |

Column A - Rel. Exp. (%) Ag3204, Run 167994669

TABLE MD
Panel 4D

| Tissue Name | A |
|---|---|
| Secondary Th1 act | 5.0 |
| Secondary Th2 act | 3.3 |
| Secondary Tr1 act | 4.0 |
| Secondary Th1 rest | 0.7 |
| Secondary Th2 rest | 0.6 |
| Secondary Tr1 rest | 0.2 |
| Primary Th1 act | 7.4 |
| Primary Th2 act | 4.6 |
| Primary Tr1 act | 5.4 |
| Primary Th1 rest | 1.9 |
| Primary Th2 rest | 1.1 |
| Primary Tr1 rest | 1.8 |
| CD45RA CD4 lymphocyte act | 1.4 |
| CD45RO CD4 lymphocyte act | 3.6 |
| CD8 lymphocyte act | 3.8 |
| Secondary CD8 lymphocyte rest | 1.8 |
| Secondary CD8 lymphocyte act | 2.5 |
| CD4 lymphocyte none | 0.6 |
| 2ry Th1/Th2/Tr1_anti-CD95 CH11 | 0.8 |
| LAK cells rest | 2.2 |
| LAK cells IL-2 | 2.1 |
| LAK cells IL-2 + IL-12 | 2.4 |
| LAK cells IL-2 + IFN gamma | 2.9 |
| LAK cells IL-2 + IL-18 | 2.4 |
| LAK cells PMA/ionomycin | 1.2 |
| NK Cells IL-2 rest | 1.4 |
| Two Way MLR 3 day | 1.5 |
| Two Way MLR 5 day | 1.6 |
| Two Way MLR 7 day | 1.4 |
| PBMC rest | 0.0 |
| PBMC PWM | 6.7 |
| PBMC PHA-L | 3.4 |
| Ramos (B cell) none | 3.6 |
| Ramos (B cell) ionomycin | 10.7 |
| B lymphocytes PWM | 9.4 |
| B lymphocytes CD40L and IL-4 | 2.1 |
| EOL-1 dbcAMP | 2.2 |
| EOL-1 dbcAMP PMA/ionomycin | 0.8 |
| Dendritic cells none | 27.0 |
| Dendritic cells LPS | 28.9 |
| Dendritic cells anti-CD40 | 41.8 |
| Monocytes rest | 0.9 |
| Monocytes LPS | 0.2 |
| Macrophages rest | 22.5 |
| Macrophages LPS | 0.8 |
| HUVEC none | 2.8 |
| HUVEC starved | 7.4 |
| HUVEC IL-1beta | 1.2 |
| HUVEC IFN gamma | 2.0 |
| HUVEC TNF alpha + IFN gamma | 1.0 |
| HUVEC TNF alpha + IL4 | 2.9 |
| HUVEC IL-11 | 2.5 |
| Lung Microvascular EC none | 2.9 |
| Lung Microvascular EC TNFalpha + IL-1beta | 2.4 |
| Microvascular Dermal EC none | 3.6 |
| Microsvasular Dermal EC TNFalpha + IL-1beta | 2.8 |
| Bronchial epithelium TNFalpha + IL1beta | 2.5 |
| Small airway epithelium none | 2.9 |
| Small airway epithelium TNFalpha + IL-1beta | 8.8 |
| Coronery artery SMC rest | 0.6 |
| Coronery artery SMC TNFalpha + IL-1beta | 0.1 |
| Astrocytes rest | 4.8 |
| Astrocytes TNFalpha + IL-1beta | 3.6 |
| KU-812 (Basophil) rest | 6.6 |
| KU-812 (Basophil) PMA/ionomycin | 10.2 |
| CCD1106 (Keratinocytes) none | 4.0 |
| CCD1106 (Keratinocytes) TNFalpha + IL-1beta | 1.8 |
| Liver cirrhosis | 1.1 |
| Lupus kidney | 0.4 |
| NCI-H292 none | 7.0 |
| NCI-H292 IL-4 | 8.8 |
| NCI-H292 IL-9 | 7.4 |
| NCI-H292 IL-13 | 3.9 |
| NCI-H292 IFN gamma | 4.1 |
| HPAEC none | 1.8 |
| HPAEC TNF alpha + IL-1 beta | 2.0 |
| Lung fibroblast none | 1.4 |
| Lung fibroblast TNF alpha + IL-1 beta | 0.6 |
| Lung fibroblast IL-4 | 2.4 |
| Lung fibroblast IL-9 | 2.8 |
| Lung fibroblast IL-13 | 1.7 |
| Lung fibroblast IFN gamma | 1.6 |
| Dermal fibroblast CCD1070 rest | 3.8 |
| Dermal fibroblast CCD1070 TNF alpha | 4.1 |
| Dermal fibroblast CCD1070 IL-1 beta | 0.4 |
| Dermal fibroblast IFN gamma | 1.0 |
| Dermal fibroblast IL-4 | 2.8 |
| IBD Colitis 2 | 0.1 |
| IBD Crohn's | 6.0 |
| Colon | 100.0 |
| Lung | 2.0 |
| Thymus | 11.6 |
| Kidney | 1.6 |

Column A - Rel. Exp. (%) Ag3204, Run 164389446

CNS_neurodegeneration_v1.0 Summary: Data obtained from RTQPCR assays performed for this panel was analyized using multivariate analysis (ANOVA). The multivariate results obtained analyzing Ag3204 shows this gene is found to be significantly (p=0.0008) downregulated in the temporal cortex of Alzheimer's disease patients when compared to controls. A close homolog of this gene has been shown to mediate neurotoxicity via amyloid beta binding. This gene may therefore be an excellent drug target for the treatment of Alzheimer's disease, specifically for blocking amyloid beta induced neuronal death. Results from a second experiment with the same probe and primer are not included. The amp plot indicates there were experimental difficulties with this run.

References:

He X Y, et al. J Biol Chem Apr. 1, 1998;273(17):10741-6

Panel 1.3D Summary: Ag3204 The CG57341-01 gene is expressed at a low level in most of the cancer cell lines and normal tissues. There appears to be significantly higher expression in colon, lung, breast and ovarian cancer cell lines with the highest expression shown by a colon cancer cell line (CT=30.94). Thus, therapeutic inhibition of this gene product, through the use of small molecule drugs, might be of utility in the treatment of the above listed cancer types.

Among tissues with metabolic function, this gene has low levels of expression in pancreas, thyroid, pituitary, adult and fetal heart, adult and fetal liver, adult and fetal skeletal muscle, and adipose. This gene product may be a small molecule target for the treatment of metabolic and endocrine disease, including the thyroidopathies, Types 1 and 2 diabetes and obesity.

Panel 4D Summary: Ag3204 The CG57341-01 transcript is expressed at significant levels in the colon and in some types of antigen presenting cells (APC'S) including activated dendritic cells, resting macrophages, and activated B cells. This pattern of expression suggests that the protein encoded by this transcript may be involved in gut immunity, particularly in the function or maintenance of APC's. This transcript encodes a putative reductase. Therefore, regulation of reductase expression could function by modulating gut immunity and be important in the treatment of inflammatory bowel diseases.

N. CG57460-01: N-Acetyltransferase Camello 2

Expression of gene CG57460-01 was assessed using the primer-probe sets Ag3273 and Ag3322, described in Tables NA and NB. Results of the RTQ-PCR runs are shown in Tables NC, ND and NE.

TABLE NA

Probe Name Ag3273

| | | | | |
|---|---|---|---|---|
| | | | Start | SEQ ID |
| Primers | Sequences | Length | Position | No |

| | | | | |
|---|---|---|---|---|
| Forward | 5′-cgctactactacagccgcaa-3′ | 20 | 205 | 152 |
| Probe | TET-5′-gtgatccgcgcctacctggagtg-3′-TAMRA | 23 | 226 | 153 |
| Reverse | 5′-gggcggcttcatgtagtact-3′ | 20 | 281 | 154 |

TABLE NB

Probe Name Ag3322

| | | | | |
|---|---|---|---|---|
| | | | Start | SEQ ID |
| Primers | Sequences | Length | Position | No |

| | | | | |
|---|---|---|---|---|
| Forward | 5′-cgctactactacagccgcaa-3′ | 20 | 205 | 155 |
| Probe | TET-5′-gtgatccgcgcctacctggagtg-3′-TAMRA | 23 | 226 | 156 |
| Reverse | 5′-gggcggcttcatgtagtact-3′ | 20 | 281 | 157 |

TABLE NC
CNS_neurodegeneration_v1.0

| Tissue Name | A | B |
|---|---|---|
| AD 1 Hippo | 15.7 | 18.6 |
| AD 2 Hippo | 28.9 | 23.2 |
| AD 3 Hippo | 11.0 | 11.0 |
| AD 4 Hippo | 10.2 | 13.8 |
| AD 5 hippo | 100.0 | 100.0 |
| AD 6 Hippo | 21.8 | 22.4 |
| Control 2 Hippo | 27.0 | 27.9 |
| Control 4 Hippo | 22.5 | 21.8 |
| Control (Path) 3 Hippo | 8.4 | 9.0 |
| AD 1 Temporal Ctx | 16.5 | 15.1 |
| AD 2 Temporal Ctx | 29.3 | 26.2 |
| AD 3 Temporal Ctx | 10.9 | 12.9 |
| AD 4 Temporal Ctx | 22.5 | 20.3 |
| AD 5 Inf Temporal Ctx | 57.0 | 59.5 |
| AD 5 Sup Temporal Ctx | 30.6 | 26.8 |
| AD 6 Inf Temporal Ctx | 27.2 | 30.1 |
| AD 6 Sup Temporal Ctx | 34.2 | 37.4 |
| Control 1 Temporal Ctx | 12.2 | 11.1 |
| Control 2 Temporal Ctx | 43.2 | 39.8 |
| Control 3 Temporal Ctx | 16.7 | 18.3 |
| Control 4 Temporal Ctx | 18.3 | 19.8 |
| Control (Path) 1 Temporal Ctx | 51.4 | 48.0 |
| Control (Path) 2 Temporal Ctx | 39.2 | 45.7 |
| Control (Path) 3 Temporal Ctx | 12.2 | 12.7 |
| Control (Path) 4 Temporal Ctx | 40.3 | 35.4 |
| AD 1 Occipital Ctx | 20.9 | 18.8 |
| AD 2 Occipital Ctx (Missing) | 0.0 | 0.0 |
| AD 3 Occipital Ctx | 15.2 | 13.1 |
| AD 4 Occipital Ctx | 22.1 | 23.2 |
| AD 5 Occipital Ctx | 18.4 | 18.4 |
| AD 6 Occipital Ctx | 46.3 | 48.3 |
| Control 1 Occipital Ctx | 8.0 | 11.2 |
| Control 2 Occipital Ctx | 80.1 | 82.4 |
| Control 3 Occipital Ctx | 26.2 | 27.2 |
| Control 4 Occipital Ctx | 14.8 | 14.1 |
| Control (Path) 1 Occipital Ctx | 70.2 | 68.8 |
| Control (Path) 2 Occipital Ctx | 21.6 | 20.6 |
| Control (Path) 3 Occipital Ctx | 8.8 | 7.4 |
| Control (Path) 4 Occipital Ctx | 36.1 | 34.2 |
| Control 1 Parietal Ctx | 14.1 | 13.7 |
| Control 2 Parietal Ctx | 33.9 | 36.9 |
| Control 3 Parietal Ctx | 33.0 | 28.7 |
| Control (Path) 1 Parietal Ctx | 63.7 | 67.8 |
| Control (Path) 2 Parietal Ctx | 30.6 | 30.4 |
| Control (Path) 3 Parietal Ctx | 8.1 | 9.7 |
| Control (Path) 4 Parietal Ctx | 64.2 | 59.5 |

Column A - Rel. Exp. (%) Ag3273, Run 210038591
Column B - Rel. Exp. (%) Ag3273, Run 230512515

TABLE ND
General_screening_panel_v1.4

| Tissue Name | A |
|---|---|
| Adipose | 7.1 |
| Melanoma* Hs688(A).T | 0.0 |
| Melanoma* Hs688(B).T | 0.0 |
| Melanoma* M14 | 17.6 |
| Melanoma* LOXIMVI | 0.9 |
| Melanoma* SK-MEL-5 | 4.2 |
| Squamous cell carcinoma SCC-4 | 0.2 |
| Testis Pool | 13.5 |
| Prostate ca.* (bone met) PC-3 | 2.7 |
| Prostate Pool | 0.2 |
| Placenta | 0.0 |
| Uterus Pool | 0.0 |
| Ovarian ca. OVCAR-3 | 5.4 |
| Ovarian ca. SK-OV-3 | 6.8 |
| Ovarian ca. OVCAR-4 | 1.6 |
| Ovarian ca. OVCAR-5 | 2.1 |
| Ovarian ca. IGROV-1 | 7.7 |
| Ovarian ca. OVCAR-8 | 9.6 |
| Ovary | 0.8 |
| Breast ca. MCF-7 | 1.7 |
| Breast ca. MDA-MB-231 | 0.8 |
| Breast ca. BT 549 | 7.7 |
| Breast ca. T47D | 9.3 |
| Breast ca. MDA-N | 0.0 |
| Breast Pool | 0.4 |
| Trachea | 0.2 |
| Lung | 0.0 |
| Fetal Lung | 0.3 |
| Lung ca. NCI-N417 | 5.1 |
| Lung ca. LX-1 | 0.4 |
| Lung ca. NCI-H146 | 12.2 |
| Lung ca. SHP-77 | 2.4 |
| Lung ca. A549 | 3.4 |
| Lung ca. NCI-H526 | 12.0 |
| Lung ca. NCI-H23 | 5.8 |
| Lung ca. NCI-H460 | 2.3 |
| Lung ca. HOP-62 | 1.0 |
| Lung ca. NCI-H522 | 7.4 |
| Liver | 0.0 |
| Fetal Liver | 0.1 |
| Liver ca. HepG2 | 0.3 |
| Kidney Pool | 0.2 |
| Fetal Kidney | 0.7 |
| Renal ca. 786-0 | 0.2 |
| Renal ca. A498 | 1.0 |
| Renal ca. ACHN | 4.5 |
| Renal ca. UO-31 | 3.7 |
| Renal ca. TK-10 | 4.9 |
| Bladder | 0.9 |
| Gastric ca. (liver met.) NCI-N87 | 2.4 |
| Gastric ca. KATO III | 0.0 |
| Colon ca. SW-948 | 0.0 |
| Colon ca. SW480 | 2.7 |
| Colon ca.* (SW480 met) SW620 | 1.0 |
| Colon ca. HT29 | 0.1 |
| Colon ca. HCT-116 | 6.0 |
| Colon ca. CaCo-2 | 2.0 |
| Colon cancer tissue | 0.6 |
| Colon ca. SW1116 | 0.4 |
| Colon ca. Colo-205 | 0.0 |
| Colon ca. SW-48 | 0.0 |
| Colon Pool | 0.4 |
| Small Intestine Pool | 0.1 |
| Stomach Pool | 0.3 |
| Bone Marrow Pool | 0.1 |
| Fetal Heart | 100.0 |
| Heart Pool | 0.2 |
| Lymph Node Pool | 0.0 |
| Fetal Skeletal Muscle | 0.3 |
| Skeletal Muscle Pool | 1.3 |
| Spleen Pool | 0.0 |
| Thymus Pool | 0.4 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.2 |
| CNS cancer (astro) SNB-75 | 0.6 |
| CNS cancer (glio) SNB-19 | 7.1 |
| CNS cancer (glio) SF-295 | 1.8 |
| Brain (Amygdala) Pool | 31.4 |
| Brain (cerebellum) | 32.3 |
| Brain (fetal) | 9.3 |
| Brain (Hippocampus) Pool | 22.5 |
| Cerebral Cortex Pool | 40.3 |
| Brain (Substantia nigra) Pool | 57.4 |
| Brain (Thalamus) Pool | 36.6 |
| Brain (whole) | 24.8 |
| Spinal Cord Pool | 20.2 |
| Adrenal Gland | 0.0 |
| Pituitary gland Pool | 2.2 |
| Salivary Gland | 0.2 |
| Thyroid (female) | 1.1 |
| Pancreatic ca. CAPAN2 | 0.0 |
| Pancreas Pool | 0.6 |

Column A - Rel. Exp. (%) Ag3273, Run 215775405

TABLE NE
Panel 4D

| Tissue Name | A |
|---|---|
| Secondary Th1 act | 1.0 |
| Secondary Th2 act | 2.5 |
| Secondary Tr1 act | 3.7 |
| Secondary Th1 rest | 5.0 |
| Secondary Th2 rest | 0.9 |
| Secondary Tr1 rest | 6.9 |
| Primary Th1 act | 6.2 |
| Primary Th2 act | 5.9 |
| Primary Tr1 act | 9.5 |
| Primary Th1 rest | 3.1 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 19.1 |
| LAK cells rest | 0.0 |
| LAK cells IL-2 | 1.2 |
| LAK cells IL-2 + IL-12 | 2.5 |
| LAK cells IL-2 + IFN gamma | 4.1 |
| LAK cells IL-2 + IL-18 | 0.0 |
| LAK cells PMA/ionomycin | 0.0 |
| NK Cells IL-2 rest | 0.0 |
| Two Way MLR 3 day | 1.1 |
| Two Way MLR 5 day | 0.0 |
| Two Way MLR 7 day | 0.0 |
| PBMC rest | 0.0 |
| PBMC PWM | 1.2 |
| PBMC PHA-L | 0.0 |
| Ramos (B cell) none | 0.0 |
| Ramos (B cell) ionomycin | 0.0 |
| B lymphocytes PWM | 3.5 |
| B lymphocytes CD40L and IL-4 | 0.0 |
| EOL-1 dbcAMP | 100.0 |
| EOL-1 dbcAMP PMA/ionomycin | 25.9 |
| Dendritic cells none | 3.8 |
| Dendritic cells LPS | 2.6 |
| Dendritic cells anti-CD40 | 5.0 |
| Monocytes rest | 0.0 |
| Monocytes LPS | 0.0 |
| Macrophages rest | 4.2 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 2.9 |
| Small airway epithelium TNFalpha + IL-1beta | 0.0 |
| Coronery artery SMC rest | 1.8 |
| Coronery artery SMC TNFalpha + IL-1beta | 0.0 |
| Astrocytes rest | 16.5 |
| Astrocytes TNFalpha + IL-1beta | 10.4 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 12.7 |
| Lupus kidney | 3.9 |
| NCI-H292 none | 10.8 |
| NCI-H292 IL-4 | 23.2 |
| NCI-H292 IL-9 | 22.4 |
| NCI-H292 IL-13 | 10.3 |
| NCI-H292 IFN gamma | 18.0 |
| HPAEC none | 0.0 |
| HPAEC TNF alpha + IL-1 beta | 0.0 |
| Lung fibroblast none | 1.9 |
| Lung fibroblast TNF alpha + IL-1 beta | 0.0 |
| Lung fibroblast IL-4 | 3.8 |
| Lung fibroblast IL-9 | 0.5 |
| Lung fibroblast IL-13 | 5.7 |
| Lung fibroblast IFN gamma | 2.9 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 2.8 |
| Dermal fibroblast IL-4 | 4.8 |
| IBD Colitis 2 | 0.0 |
| IBD Crohn's | 0.0 |
| Colon | 37.4 |
| Lung | 8.4 |
| Thymus | 40.9 |
| Kidney | 6.3 |

Column A - Rel. Exp. (%) Ag3273, Run 165338992

CNS_neurodegeneration_v1.0 Summary: Data obtained from RTQPCR assays performed for this panel was analyized using multivariate analysis (ANOVA). The multivariate results obtained analyzing Ag3273 shows the two experiments with the same probe and primer set produced results that are in excellent agreement. This panel confirms the expression of this gene at low to moderate levels in the brains of an independent group of individuals. Expression of this gene is found to be down-regulated in the temporal cortex of Alzheimer's disease patients. Therefore, up-regulation of this gene or its protein product, or treatment with specific agonists for this protein, may be of use in reversing the dementia/memory loss associated with Alzheimer's disease and neuronal death.

General_screening_panel_v1.4 Summary: Ag3273 Highest expression of the CG57460-01 gene is seen in fetal heart (CT=28.6). In addition, this gene is expressed at much higher levels in fetal heart when compared to expression in the adult heart (CT=38). Thus, expression of this gene may be used to differentiate between disorders or predispositions that differ between the fetal and adult source of this tissue. In addition, the higher expression in fetal heart suggests that this protein product may be involved in the development of this organ. Therefore, therapeutic modulation of the expression or function of this gene may be useful in the treatment of heart disease.

In addition, expression of this gene appears to be upregulated in a number of cancer cell lines when compared to the normal tissues. Specifically, expression of this gene appears to be higher in ovarian, breast, lung and renal cancer cell lines when compared to their respective normal tissues. Therefore, therapeutic modulation of the activity of this gene or its protein product, using small molecule drugs, antibodies, or protein therapeutics, may be of benefit in the treatment of ovarian, breast, lung and renal cancer. The CG57460-01 gene encodes a transmembrane protein with homology to N-acetyltransferase Camello 2, a protein involved in cellular adhesion (ref. 1).

References:

Popsueva A E, et al. Dev Biol Jun. 15, 2001;234(2):483-96 (PMID: 11397015)

Panel 4D Summary: Ag3273 Highest expression of the CG57460-01 is seen in eosinophils. In addition, differential expression is observed in the eosinophil cell line EOL-1 under resting conditions over that in EOL-1 cells stimulated by phorbol ester and ionomycin. Thus, this gene may be involved in eosinophil function. Therefore, therapeutic modulation of the expression or function of this gene may reduce eosinophil activation and be useful in the treatment of asthma and allergies.

In addition, significant expression in normal colon and thymus suggest a role for this gene in the normal homeostasis of these tissues. Therefore, therapeutic modulation of the expression or function of this gene may modulate immune function (T cell development) and be important for organ transplant, AIDS treatment or post chemotherapy immune reconstitiution. Furthermore, since expression of this gene is decreased in colon samples from patients with IBD colitis and Crohn's disease relative to normal colon, therapeutic modulation of the activity of the protein encoded by this gene may be useful in the treatment of inflammatory bowel disease.

O. CG57570-01: Cation Transporter

Expression of gene CG57570-01 was assessed using the primer-probe set Ag3288, described in Table OA. Results of the RTQ-PCR runs are shown in Tables OB, OC and OD.

TABLE OA

Probe Name Ag3288

| | | | | |
|---|---|---|---|---|
| | | | Start | SEQ ID |
| Primers | Sequences | Length | Position | No |

| | | | | |
|---|---|---|---|---|
| Forward | 5′-tctacaccatcagctgtatgca-3′ | 22 | 1380 | 158 |
| Probe | TET-5′-caccaccctcacactcatcttcatca-3′-TAMRA | 26 | 1411 | 159 |
| Reverse | 5′-gcagtgcagctgtcatatagaa-3′ | 22 | 1439 | 160 |

TABLE OB
CNS_neurodegeneration_v1.0

| Tissue Name | A | B |
|---|---|---|
| AD 1 Hippo | 26.6 | 31.0 |
| AD 2 Hippo | 43.5 | 48.0 |
| AD 3 Hippo | 15.0 | 14.8 |
| AD 4 Hippo | 13.0 | 11.5 |
| AD 5 hippo | 97.3 | 100.0 |
| AD 6 Hippo | 100.0 | 90.1 |
| Control 2 Hippo | 29.3 | 28.9 |
| Control 4 Hippo | 33.7 | 41.2 |
| Control (Path) 3 Hippo | 22.8 | 27.9 |
| AD 1 Temporal Ctx | 34.2 | 35.8 |
| AD 2 Temporal Ctx | 47.3 | 52.1 |
| AD 3 Temporal Ctx | 14.0 | 18.6 |
| AD 4 Temporal Ctx | 34.6 | 46.7 |
| AD 5 Inf Temporal Ctx | 80.1 | 100.0 |
| AD 5 Sup Temporal Ctx | 64.2 | 85.9 |
| AD 6 Inf Temporal Ctx | 68.8 | 87.1 |
| AD 6 Sup Temporal Ctx | 66.4 | 87.1 |
| Control 1 Temporal Ctx | 11.3 | 16.2 |
| Control 2 Temporal Ctx | 33.0 | 36.3 |
| Control 3 Temporal Ctx | 19.2 | 18.9 |
| Control 4 Temporal Ctx | 14.7 | 17.8 |
| Control (Path) 1 Temporal Ctx | 32.3 | 50.7 |
| Control (Path) 2 Temporal Ctx | 36.9 | 13.2 |
| Control (Path) 3 Temporal Ctx | 13.3 | 15.1 |
| Control (Path) 4 Temporal Ctx | 33.7 | 36.3 |
| AD 1 Occipital Ctx | 25.7 | 15.5 |
| AD 2 Occipital Ctx (Missing) | 0.0 | 0.0 |
| AD 3 Occipital Ctx | 16.5 | 15.8 |
| AD 4 Occipital Ctx | 26.1 | 35.1 |
| AD 5 Occipital Ctx | 26.4 | 28.5 |
| AD 6 Occipital Ctx | 33.2 | 37.6 |
| Control 1 Occipital Ctx | 8.8 | 10.4 |
| Control 2 Occipital Ctx | 51.8 | 59.9 |
| Control 3 Occipital Ctx | 28.5 | 12.8 |
| Control 4 Occipital Ctx | 16.5 | 17.8 |
| Control (Path) 1 Occipital Ctx | 66.0 | 82.4 |
| Control (Path) 2 Occipital Ctx | 11.3 | 14.4 |
| Control (Path) 3 Occipital Ctx | 9.5 | 8.5 |
| Control (Path) 4 Occipital Ctx | 13.9 | 17.7 |
| Control 1 Parietal Ctx | 11.1 | 15.8 |
| Control 2 Parietal Ctx | 48.0 | 66.0 |
| Control 3 Parietal Ctx | 17.0 | 22.2 |
| Control (Path) 1 Parietal Ctx | 55.5 | 66.4 |
| Control (Path) 2 Parietal Ctx | 30.6 | 34.2 |
| Control (Path) 3 Parietal Ctx | 14.3 | 11.3 |
| Control (Path) 4 Parietal Ctx | 37.1 | 43.2 |

Column A - Rel. Exp. (%) Ag3288, Run 210058660
Column B - Rel. Exp. (%) Ag3288, Run 229929907

TABLE OC
General_screening_panel_v1.4

| Tissue Name | A |
|---|---|
| Adipose | 15.8 |
| Melanoma* Hs688(A).T | 38.4 |
| Melanoma* Hs688(B).T | 51.4 |
| Melanoma* M14 | 43.5 |
| Melanoma* LOXIMVI | 25.0 |
| Melanoma* SK-MEL-5 | 52.9 |
| Squamous cell carcinoma SCC-4 | 16.5 |
| Testis Pool | 44.4 |
| Prostate ca.* (bone met) PC-3 | 57.4 |
| Prostate Pool | 16.2 |
| Placenta | 4.3 |
| Uterus Pool | 4.9 |
| Ovarian ca. OVCAR-3 | 37.9 |
| Ovarian ca. SK-OV-3 | 39.0 |
| Ovarian ca. OVCAR-4 | 18.0 |
| Ovarian ca. OVCAR-5 | 63.3 |
| Ovarian ca. IGROV-1 | 14.5 |
| Ovarian ca. OVCAR-8 | 12.1 |
| Ovary | 17.2 |
| Breast ca. MCF-7 | 32.5 |
| Breast ca. MDA-MB-231 | 47.3 |
| Breast ca. BT 549 | 58.2 |
| Breast ca. T47D | 73.7 |
| Breast ca. MDA-N | 12.6 |
| Breast Pool | 14.6 |
| Trachea | 14.6 |
| Lung | 7.5 |
| Fetal Lung | 63.7 |
| Lung ca. NCI-N417 | 2.7 |
| Lung ca. LX-1 | 19.5 |
| Lung ca. NCI-H146 | 4.9 |
| Lung ca. SHP-77 | 12.0 |
| Lung ca. A549 | 24.8 |
| Lung ca. NCI-H526 | 8.8 |
| Lung ca. NCI-H23 | 23.8 |
| Lung ca. NCI-H460 | 29.5 |
| Lung ca. HOP-62 | 23.0 |
| Lung ca. NCI-H522 | 36.1 |
| Liver | 0.5 |
| Fetal Liver | 27.2 |
| Liver ca. HepG2 | 0.2 |
| Kidney Pool | 23.0 |
| Fetal Kidney | 21.2 |
| Renal ca. 786-0 | 32.1 |
| Renal ca. A498 | 17.6 |
| Renal ca. ACHN | 36.1 |
| Renal ca. UO-31 | 21.0 |
| Renal ca. TK-10 | 24.0 |
| Bladder | 26.8 |
| Gastric ca. (liver met.) NCI-N87 | 55.1 |
| Gastric ca. KATO III | 53.6 |
| Colon ca. SW-948 | 19.1 |
| Colon ca. SW480 | 41.2 |
| Colon ca.* (SW480 met) SW620 | 14.4 |
| Colon ca. HT29 | 9.5 |
| Colon ca. HCT-116 | 50.7 |
| Colon ca. CaCo-2 | 9.5 |
| Colon cancer tissue | 7.3 |
| Colon ca. SW1116 | 6.7 |
| Colon ca. Colo-205 | 10.5 |
| Colon ca. SW-48 | 7.3 |
| Colon Pool | 15.9 |
| Small Intestine Pool | 15.0 |
| Stomach Pool | 9.6 |
| Bone Marrow Pool | 9.4 |
| Fetal Heart | 72.7 |
| Heart Pool | 66.9 |
| Lymph Node Pool | 19.3 |
| Fetal Skeletal Muscle | 17.6 |
| Skeletal Muscle Pool | 63.7 |
| Spleen Pool | 11.0 |
| Thymus Pool | 7.5 |
| CNS cancer (glio/astro) U87-MG | 30.8 |
| CNS cancer (glio/astro) U-118-MG | 44.1 |
| CNS cancer (neuro; met) SK-N-AS | 37.9 |
| CNS cancer (astro) SF-539 | 33.2 |
| CNS cancer (astro) SNB-75 | 34.6 |
| CNS cancer (glio) SNB-19 | 18.9 |
| CNS cancer (glio) SF-295 | 64.2 |
| Brain (Amygdala) Pool | 13.4 |
| Brain (cerebellum) | 100.0 |
| Brain (fetal) | 28.5 |
| Brain (Hippocampus) Pool | 21.5 |
| Cerebral Cortex Pool | 22.8 |
| Brain (Substantia nigra) Pool | 19.2 |
| Brain (Thalamus) Pool | 26.8 |
| Brain (whole) | 40.9 |
| Spinal Cord Pool | 19.8 |
| Adrenal Gland | 40.3 |
| Pituitary gland Pool | 5.8 |
| Salivary Gland | 6.4 |
| Thyroid (female) | 17.2 |
| Pancreatic ca. CAPAN2 | 14.8 |
| Pancreas Pool | 22.8 |

Column A - Rel. Exp. (%) Ag3288, Run 216516909

TABLE OD
Panel 4D

| Tissue Name | A |
|---|---|
| Secondary Th1 act | 15.0 |
| Secondary Th2 act | 15.8 |
| Secondary Tr1 act | 15.0 |
| Secondary Th1 rest | 3.0 |
| Secondary Th2 rest | 8.5 |
| Secondary Tr1 rest | 4.4 |
| Primary Th1 act | 28.5 |
| Primary Th2 act | 19.5 |
| Primary Tr1 act | 24.0 |
| Primary Th1 rest | 11.5 |
| Primary Th2 rest | 8.5 |
| Primary Tr1 rest | 4.6 |
| CD45RA CD4 lymphocyte act | 12.2 |
| CD45RO CD4 lymphocyte act | 21.2 |
| CD8 lymphocyte act | 14.2 |
| Secondary CD8 lymphocyte rest | 22.1 |
| Secondary CD8 lymphocyte act | 10.8 |
| CD4 lymphocyte none | 5.1 |
| 2ry Th1/Th2/Tr1_anti-CD95 CH11 | 4.0 |
| LAK cells rest | 4.8 |
| LAK cells IL-2 | 10.6 |
| LAK cells IL-2 + IL-12 | 15.7 |
| LAK cells IL-2 + IFN gamma | 19.3 |
| LAK cells IL-2 + IL-18 | 14.2 |
| LAK cells PMA/ionomycin | 5.7 |
| NK Cells IL-2 rest | 7.9 |
| Two Way MLR 3 day | 11.5 |
| Two Way MLR 5 day | 9.7 |
| Two Way MLR 7 day | 6.7 |
| PBMC rest | 3.6 |
| PBMC PWM | 61.6 |
| PBMC PHA-L | 31.6 |
| Ramos (B cell) none | 13.7 |
| Ramos (B cell) ionomycin | 94.6 |
| B lymphocytes PWM | 63.7 |
| B lymphocytes CD40L and IL-4 | 100.0 |
| EOL-1 dbcAMP | 3.9 |
| EOL-1 dbcAMP PMA/ionomycin | 6.7 |
| Dendritic cells none | 2.2 |
| Dendritic cells LPS | 1.9 |
| Dendritic cells anti-CD40 | 0.8 |
| Monocytes rest | 0.5 |
| Monocytes LPS | 5.8 |
| Macrophages rest | 1.0 |
| Macrophages LPS | 7.2 |
| HUVEC none | 10.6 |
| HUVEC starved | 17.3 |
| HUVEC IL-1beta | 10.7 |
| HUVEC IFN gamma | 9.3 |
| HUVEC TNF alpha + IFN gamma | 20.9 |
| HUVEC TNF alpha + IL4 | 14.9 |
| HUVEC IL-11 | 6.6 |
| Lung Microvascular EC none | 9.3 |
| Lung Microvascular EC TNFalpha + IL-1beta | 58.2 |
| Microvascular Dermal EC none | 22.4 |
| Microsvasular Dermal EC TNFalpha + IL-1beta | 63.3 |
| Bronchial epithelium TNFalpha + IL1beta | 20.6 |
| Small airway epithelium none | 3.8 |
| Small airway epithelium TNFalpha + IL-1beta | 48.6 |
| Coronery artery SMC rest | 11.7 |
| Coronery artery SMC TNFalpha + IL-1beta | 9.1 |
| Astrocytes rest | 16.4 |
| Astrocytes TNFalpha + IL-1beta | 12.9 |
| KU-812 (Basophil) rest | 16.8 |
| KU-812 (Basophil) PMA/ionomycin | 50.3 |
| CCD1106 (Keratinocytes) none | 14.0 |
| CCD1106 (Keratinocytes) TNFalpha + IL-1beta | 10.8 |
| Liver cirrhosis | 3.3 |
| Lupus kidney | 1.6 |
| NCI-H292 none | 14.6 |
| NCI-H292 IL-4 | 18.9 |
| NCI-H292 IL-9 | 16.3 |
| NCI-H292 IL-13 | 13.1 |
| NCI-H292 IFN gamma | 11.2 |
| HPAEC none | 11.5 |
| HPAEC TNF alpha + IL-1 beta | 31.0 |
| Lung fibroblast none | 13.9 |
| Lung fibroblast TNF alpha + IL-1 beta | 13.6 |
| Lung fibroblast IL-4 | 28.1 |
| Lung fibroblast IL-9 | 22.2 |
| Lung fibroblast IL-13 | 13.5 |
| Lung fibroblast IFN gamma | 23.7 |
| Dermal fibroblast CCD1070 rest | 30.4 |
| Dermal fibroblast CCD1070 TNF alpha | 49.0 |
| Dermal fibroblast CCD1070 IL-1 beta | 15.6 |
| Dermal fibroblast IFN gamma | 6.4 |
| Dermal fibroblast IL-4 | 14.2 |
| IBD Colitis 2 | 1.6 |
| IBD Crohn's | 0.9 |
| Colon | 5.9 |
| Lung | 13.8 |
| Thymus | 48.0 |
| Kidney | 20.4 |

Column A - Rel. Exp. (%) Ag3288, Run 165007638

CNS_neurodegeneration_v1.0 Summary: Data obtained from RTQPCR assays performed for this panel was analyized using multivariate analysis (ANOVA). The multivariate results obtained analyzing Ag3288 shows results from two experiments using the same probe/primer set are in excellent agreement. This gene was found to upregulated in the temporal cortex of Alzheimer's disease (AD) patients (p=0.0007 when analyzed by ANCOVA); the temporal cortex shows marked neuronal loss in the early to middle stages of AD. Upregulation of this gene expression, however, was not apparent in the occipital cortex, where neuronal degeneration does not occur in AD. Taken together, these data suggest that the protein encoded by this gene is involved in the pathologic process of Alzheimer's disease, making this an excellent small molecule drug target.

This gene encodes a protein with homology to cation transporters. For example, iron transporters in the brain have been shown to play an important role in age-related neurodegenerative diseases, including Parkinson's Disease, Alzheimer's disease, Huntington's disease and amyotrophic lateral sclerosis (Qian Z M, et al. Brain Res Brain Res Rev August 1998;27(3):257-67).

General_screening_panel_v1.4 Summary: Ag3288 The CG57570-01 gene is expressed at high to moderate levels across almost all samples in this panel, with highest expression in the cerebellum (CT=26.7). This gene is also moderately expressed in all other regions of the CNS examined, including in amygdala, substantia nigra, thalamus, cerebral cortex, and spinal cord, suggesting that this gene may play a role in central nervous system disorders such as Alzheimer's disease, Parkinson's disease, epilepsy, multiple sclerosis, schizophrenia and depression.

This gene is also expressed in a number of tissues with metabolic or endocrine function, including adipose, adrenal gland, gastrointestinal tract, pancreas, skeletal muscle and thyroid. Therefore, therapeutic modulation of the activity of this gene may prove useful in the treatment of endocrine/metabolically related diseases, such as obesity and diabetes. Interestingly, this gene is differentially expressed in adult liver (CT=34) vs fetal liver (CT=29).

In addition, there is substantial expression of this gene associated with cancer cell lines. Therefore, therapeutic modulation of the activity of this gene or its protein product, through the use of small molecule drugs, protein therapeutics or antibodies, might be beneficial in the treatment of cancer.

Panel 4D Summary: Ag3288 This gene is expressed at high to moderate levels in a wide range of cell types of significance in the immune response and tissue response in health and disease, with the highest expression being detected in activated B cells (CT=25.4). Targeting this gene or its protein product with a small molecule drug or antibody therapeutic may modulate the functions of cells of the immune system and in particular of B cells as well as resident tissue cells and lead to improvement of the symptoms of patients suffering from rheumatoid diseases or B hyperglobulinemia and also other autoimmune disorders.

P. CG57758-02: Sodium/Lithium-dependent Dicarboxylate Transporter

Expression of gene CG57758-02 was assessed using the primer-probe sets Ag3326 and Ag3692, described in Tables PA and PB. Results of the RTQ-PCR runs are shown in Tables PC, PD, PE and PF.

TABLE PA

Probe Name Ag3326

| | | | | |
|---|---|---|---|---|
| | | | Start | SEQ ID |
| Primers | Sequences | Length | Position | No |

| | | | | |
|---|---|---|---|---|
| Forward | 5′-ccatttactggtgcacagaagt-3′ | 22 | 167 | 161 |
| Probe | TET-5′-atccctctggctgtcacctctctcat-3′-TAMRA | 26 | 190 | 162 |
| Reverse | 5′-ggagtccagaatctggaagagt-3′ | 22 | 234 | 163 |

TABLE PB

Probe Name Ag3692

| | | | | |
|---|---|---|---|---|
| | | | Start | SEQ ID |
| Primers | Sequences | Length | Position | NO |

| | | | | |
|---|---|---|---|---|
| Forward | 5′-ccatttactggtgcacagaagt-3′ | 22 | 167 | 164 |
| Probe | TET-5′-atccctctggctgtcacctctctcat-3′-TAMRA | 26 | 190 | 165 |
| Reverse | 5′-ggagtccagaatctggaagagt-3′ | 22 | 234 | 166 |

TABLE PC
CNS_neurodegeneration_v1.0

| Tissue Name | A | B | C |
|---|---|---|---|
| AD 1 Hippo | 2.1 | 4.3 | 1.0 |
| AD 2 Hippo | 20.9 | 28.3 | 25.0 |
| AD 3 Hippo | 0.0 | 0.9 | 0.6 |
| AD 4 Hippo | 2.1 | 7.1 | 2.6 |
| AD 5 hippo | 72.7 | 97.9 | 85.3 |
| AD 6 Hippo | 13.7 | 18.3 | 5.5 |
| Control 2 Hippo | 14.5 | 20.2 | 15.2 |
| Control 4 Hippo | 11.7 | 7.4 | 5.1 |
| Control (Path) 3 Hippo | 6.7 | 4.4 | 4.5 |
| AD 1 Temporal Ctx | 4.0 | 1.7 | 2.8 |
| AD 2 Temporal Ctx | 80.7 | 50.7 | 37.4 |
| AD 3 Temporal Ctx | 3.6 | 0.0 | 1.1 |
| AD 4 Temporal Ctx | 19.5 | 30.6 | 15.2 |
| AD 5 Inf Temporal Ctx | 100.0 | 100.0 | 99.3 |
| AD 5 Sup Temporal Ctx | 32.8 | 29.1 | 33.2 |
| AD 6 Inf Temporal Ctx | 27.7 | 21.3 | 26.6 |
| AD 6 Sup Temporal Ctx | 41.8 | 53.6 | 17.0 |
| Control 1 Temporal Ctx | 12.0 | 33.9 | 18.3 |
| Control 2 Temporal Ctx | 30.1 | 49.3 | 44.4 |
| Control 3 Temporal Ctx | 38.7 | 39.5 | 33.4 |
| Control 4 Temporal Ctx | 17.6 | 25.2 | 24.1 |
| Control (Path) 1 Temporal | 69.7 | 70.7 | 49.7 |
| Ctx | | | |
| Control (Path) 2 Temporal | 35.4 | 50.7 | 33.4 |
| Ctx | | | |
| Control (Path) 3 Temporal | 8.5 | 15.3 | 12.0 |
| Ctx | | | |
| Control (Path) 4 Temporal | 31.2 | 36.6 | 52.1 |
| Ctx | | | |
| AD 1 Occipital Ctx | 2.7 | 3.0 | 0.0 |
| AD 2 Occipital Ctx | 0.0 | 0.0 | 0.0 |
| (Missing) | | | |
| AD 3 Occipital Ctx | 1.5 | 7.2 | 1.3 |
| AD 4 Occipital Ctx | 71.7 | 35.6 | 30.6 |
| AD 5 Occipital Ctx | 25.3 | 31.9 | 12.4 |
| AD 6 Occipital Ctx | 17.2 | 19.1 | 11.2 |
| Control 1 Occipital Ctx | 7.0 | 9.0 | 8.1 |
| Control 2 Occipital Ctx | 33.2 | 44.8 | 26.1 |
| Control 3 Occipital Ctx | 30.1 | 37.6 | 21.9 |
| Control 4 Occipital Ctx | 16.3 | 12.6 | 8.2 |
| Control (Path) 1 Occipital | 42.0 | 55.9 | 52.9 |
| Ctx | | | |
| Control (Path) 2 Occipital | 6.7 | 13.0 | 7.7 |
| Ctx | | | |
| Control (Path) 3 Occipital | 8.7 | 6.6 | 5.4 |
| Ctx | | | |
| Control (Path) 4 Occipital | 8.1 | 9.0 | 7.4 |
| Ctx | | | |
| Control 1 Parietal Ctx | 21.2 | 23.0 | 15.3 |
| Control 2 Parietal Ctx | 48.6 | 38.2 | 22.1 |
| Control 3 Parietal Ctx | 28.3 | 34.4 | 32.8 |
| Control (Path) 1 Parietal | 78.5 | 97.3 | 100.0 |
| Ctx | | | |
| Control (Path) 2 Parietal | 50.7 | 50.7 | 37.9 |
| Ctx | | | |
| Control (Path) 3 Parietal | 10.7 | 10.1 | 9.6 |
| Ctx | | | |
| Control (Path) 4 Parietal | 30.6 | 24.5 | 40.9 |
| Ctx | | | |

Column A - Rel. Exp. (%) Ag3326, Run 210144197
Column B - Rel. Exp. (%) Ag3692, Run 211145262
Column C - Rel. Exp. (%) Ag3692, Run 224337942

TABLE PD
General_screening_panel_v1.4

| Tissue Name | A | B |
|---|---|---|
| Adipose | 0.0 | 0.0 |
| Melanoma* Hs688(A).T | 0.0 | 0.0 |
| Melanoma* Hs688(B).T | 0.1 | 0.0 |
| Melanoma* M14 | 0.0 | 0.0 |
| Melanoma* LOXIMVI | 0.0 | 0.0 |
| Melanoma* SK-MEL-5 | 0.0 | 0.0 |
| Squamous cell carcinoma | 0.9 | 0.7 |
| SCC-4 | | |
| Testis Pool | 0.1 | 0.2 |
| Prostate ca.* (bone met) PC-3 | 0.0 | 0.0 |
| Prostate Pool | 0.0 | 0.0 |
| Placenta | 0.0 | 0.0 |
| Uterus Pool | 0.0 | 0.0 |
| Ovarian ca. OVCAR-3 | 0.0 | 0.0 |
| Ovarian ca. SK-OV-3 | 0.0 | 0.0 |
| Ovarian ca. OVCAR-4 | 0.1 | 0.0 |
| Ovarian ca. OVCAR-5 | 0.0 | 0.0 |
| Ovarian ca. IGROV-1 | 0.0 | 0.0 |
| Ovarian ca. OVCAR-8 | 2.8 | 2.2 |
| Ovary | 0.7 | 0.6 |
| Breast ca. MCF-7 | 0.0 | 0.0 |
| Breast ca. MDA-MB-231 | 0.0 | 0.0 |
| Breast ca. BT 549 | 0.6 | 0.8 |
| Breast ca. T47D | 0.0 | 0.0 |
| Breast ca. MDA-N | 0.0 | 0.0 |
| Breast Pool | 0.0 | 0.1 |
| Trachea | 0.2 | 0.1 |
| Lung | 0.0 | 0.0 |
| Fetal Lung | 0.2 | 0.1 |
| Lung ca. NCI-N417 | 0.0 | 0.0 |
| Lung ca. LX-1 | 0.0 | 0.0 |
| Lung ca. NCI-H146 | 0.0 | 0.0 |
| Lung ca. SHP-77 | 0.0 | 0.0 |
| Lung ca. A549 | 0.0 | 0.1 |
| Lung ca. NCI-H526 | 2.0 | 0.0 |
| Lung ca. NCI-H23 | 0.7 | 0.6 |
| Lung ca. NCI-H460 | 0.0 | 0.0 |
| Lung ca. HOP-62 | 0.1 | 0.2 |
| Lung ca. NCI-H522 | 0.0 | 0.0 |
| Liver | 28.7 | 24.1 |
| Fetal Liver | 100.0 | 100.0 |
| Liver ca. HepG2 | 29.5 | 26.2 |
| Kidney Pool | 0.0 | 0.0 |
| Fetal Kidney | 0.1 | 0.1 |
| Renal ca. 786-0 | 0.0 | 0.0 |
| Renal ca. A498 | 0.0 | 0.0 |
| Renal ca. ACHN | 0.0 | 0.0 |
| Renal ca. UO-31 | 0.0 | 0.0 |
| Renal ca. TK-10 | 11.4 | 12.0 |
| Bladder | 0.0 | 0.1 |
| Gastric ca. (liver met.) NCI-N87 | 0.0 | 0.0 |
| Gastric ca. KATO III | 0.0 | 0.0 |
| Colon ca. SW-948 | 0.0 | 0.0 |
| Colon ca. SW480 | 0.0 | 0.0 |
| Colon ca.* (SW480 met) SW620 | 0.0 | 0.0 |
| Colon ca. HT29 | 0.0 | 0.0 |
| Colon ca. HCT-116 | 0.0 | 0.0 |
| Colon ca. CaCo-2 | 0.0 | 0.0 |
| Colon cancer tissue | 0.1 | 0.0 |
| Colon ca. SW1116 | 0.0 | 0.0 |
| Colon ca. Colo-205 | 0.0 | 0.0 |
| Colon ca. SW-48 | 0.0 | 0.0 |
| Colon Pool | 0.6 | 0.0 |
| Small Intestine Pool | 0.1 | 0.0 |
| Stomach Pool | 0.0 | 0.0 |
| Bone Marrow Pool | 0.0 | 0.1 |
| Fetal Heart | 0.0 | 0.0 |
| Heart Pool | 0.0 | 0.0 |
| Lymph Node Pool | 0.1 | 0.0 |
| Fetal Skeletal Muscle | 0.0 | 0.0 |
| Skeletal Muscle Pool | 0.0 | 0.0 |
| Spleen Pool | 0.4 | 0.2 |
| Thymus Pool | 0.0 | 0.0 |
| CNS cancer (glio/astro) U87-MG | 0.0 | 0.0 |
| CNS cancer (glio/astro) U-118- | 0.0 | 0.0 |
| MG | | |
| CNS cancer (neuro; met) SK-N- | 0.0 | 0.0 |
| AS | | |
| CNS cancer (astro) SF-539 | 0.0 | 0.0 |
| CNS cancer (astro) SNB-75 | 0.0 | 0.0 |
| CNS cancer (glio) SNB-19 | 0.0 | 0.0 |
| CNS cancer (glio) SF-295 | 0.1 | 0.1 |
| Brain (Amygdala) Pool | 0.4 | 0.4 |
| Brain (cerebellum) | 1.4 | 1.0 |
| Brain (fetal) | 0.7 | 0.4 |
| Brain (Hippocampus) Pool | 0.5 | 0.7 |
| Cerebral Cortex Pool | 1.4 | 1.5 |
| Brain (Substantia nigra) Pool | 1.4 | 1.4 |
| Brain (Thalamus) Pool | 1.1 | 0.9 |
| Brain (whole) | 4.1 | 3.7 |
| Spinal Cord Pool | 0.1 | 0.2 |
| Adrenal Gland | 2.6 | 1.9 |
| Pituitary gland Pool | 0.0 | 0.2 |
| Salivary Gland | 40.9 | 35.1 |
| Thyroid (female) | 0.0 | 0.0 |
| Pancreatic ca. CAPAN2 | 0.5 | 0.8 |
| Pancreas Pool | 0.0 | 0.0 |

Column A - Rel. Exp. (%) Ag3326, Run 215678613
Column B - Rel. Exp. (%) Ag3692, Run 217131191

TABLE PE
Panel 4.1D

| Tissue Name | A |
|---|---|
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 4.2 |
| Primary Th1 rest | 0.0 |
| Primary Th2 rest | 0.0 |
| Primary Tr1 rest | 0.0 |
| CD45RA CD4 lymphocyte act | 3.9 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 3.2 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 11.3 |
| Microsvasular Dermal EC TNFalpha + IL-1beta | 0.0 |
| Bronchial epithelium TNFalpha + IL1beta | 28.5 |
| Small airway epithelium none | 5.7 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 3.6 |
| KU-812 (Basophil) PMA/ionomycin | 4.3 |
| CCD1106 (Keratinocytes) none | 10.7 |
| CCD1106 (Keratinocytes) TNFalpha + IL-1beta | 0.0 |
| Liver cirrhosis | 94.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.0 |
| Lung | 0.0 |
| Thymus | 2.4 |
| Kidney | 100.0 |

Column A - Rel. Exp. (%) Ag3692, Run 169987356

TABLE PF
Panel 5 Islet

| Tissue Name | A |
|---|---|
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 0.0 |
| 99167_Bayer Patient 1 | 0.3 |
| 97482_Patient-08ut_uterus | 0.0 |
| 97483_Patient-08pl_placenta | 0.0 |
| 97486_Patient-09sk_skeletal muscle | 0.0 |
| 97487_Patient-09ut_uterus | 0.0 |
| 97488_Patient-09pl_placenta | 0.0 |
| 97492_Patient-10ut_uterus | 0.0 |
| 97493_Patient-10pl_placenta | 0.0 |
| 97495_Patient-11go_adipose | 0.0 |
| 97496_Patient-11sk_skeletal muscle | 0.0 |
| 97497_Patient-11ut_uterus | 0.0 |
| 97498_Patient-11pl_placenta | 0.0 |
| 97500_Patient-12go_adipose | 0.1 |
| 97501_Patient-12sk_skeletal muscle | 0.3 |
| 97502_Patient-12ut_uterus | 0.0 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.2 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 100.0 |
| 73556_Heart_Cardiac stromal cells (primary) | 0.0 |
| 81735_Small Intestine | 39.5 |
| 72409_Kidney_Proximal Convoluted Tubule | 0.0 |
| 82685_Small intestine_Duodenum | 0.0 |
| 90650_Adrenal_Adrenocortical adenoma | 0.0 |
| 72410_Kidney_HRCE | 0.0 |
| 72411_Kidney_HRE | 0.0 |
| 73139_Uterus_Uterine smooth muscle cells | 0.0 |

Column A - Rel. Exp. (%) Ag3326, Run 242385365

CNS_neurodegeneration_v1.0 Summary: Data obtained from RTQPCR assays performed for this panel was analyized using multivariate analysis (ANOVA). The multivariate results obtained analyzing Ag3326/Ag3692 shows three experiments tested with two primer pairs (same sequence) are in excellent agreement. This gene is found to be down-regulated in the temporal cortex of Alzheimer's disease patients. Therefore, up-regulation of this gene or its protein product, or treatment with specific agonists for this gene may be of use in reversing the dementia/memory loss associated with this disease and neuronal death.

General_screening_panel_v1.4 Summary: Ag3326/Ag3692 Two experiments with the same probe and primer set produce results that are in excellent agreement. This gene is highly expressed in fetal liver (CT=26.5-27.0) and moderately expressed in adult liver and liver cancer cell line HepG2. This result agrees with the results seen in Panel 5 (expression in HepG2). These results are in agreement with published data that show a novel sodium dicarboxylate transporter in brain, choroid plexus kidney, intestine and liver (Chen X Z, et al. J Biol Chem Aug. 14, 1998;273(33):20972-81; Pajor A M, et al. Am J Physiol Cell Physiol May 2001;280(5):C1215-23).

This gene is expressed at low levels throughout the CNS, including in amygdala, substantia nigra, thalamus, cerebellum, and cerebral cortex. Therefore, this gene may play a role in central nervous system disorders such as Parkinson's disease, epilepsy, multiple sclerosis, schizophrenia and depression.

Low but significant levels of expression are also seen in the adrenal gland. Thus, this gene product may also be involved in metabolic disorders of this gland, including adrenoleuleukodystrophy and congenital adrenal hyperplasia.

Panel 4.1D Summary: Ag3692 Significant expression of this gene is seen only in kidney and a liver cirrhosis samples in this panel (CTs=34.0). These results confirm that this gene is expressed in liver derived samples.

Panel 5 Islet Summary: Ag3326—The highest expression of this gene is in liver cancer cell line HepG2 (CT=29.2). There is also moderate expression in the small intestine (CT=30.5).

Q. CG59693-01: 20 Alpha-hydroxysteroid Dehydrogenase

Expression of gene CG59693-01 was assessed using the primer-probe set Ag3562, described in Table QA. Results of the RTQ-PCR runs are shown in Tables QB, QE, QF, QG, QH, QI and QJ.

TABLE QA

Probe Name Ag3562

| | | | | |
|---|---|---|---|---|
| | | | Start | SEQ ID |
| Primers | Sequences | Length | Position | No |

| | | | | |
|---|---|---|---|---|
| Forward | 5′-ctggccaagagctacaatga-3′ | 20 | 802 | 168 |
| Probe | TET-5′-catcagacagaacgtgcaggtgtttg-3′-TAMRA | 26 | 828 | 169 |
| Reverse | 5′-aggccatctatggctttcat-3′ | 20 | 877 | 170 |

TABLE QB
Ardais Panel v.1.0

| Tissue Name | A |
|---|---|
| 136799_Lung cancer (362) | 52.9 |
| 136800_Lung NAT (363) | 0.8 |
| 136813_Lung cancer (372) | 100.0 |
| 136814_Lung NAT (373) | 0.4 |
| 136815_Lung cancer (374) | 1.1 |
| 136816_Lung NAT (375) | 1.6 |
| 136791_Lung cancer (35A) | 0.4 |
| 136795_Lung cancer (35E) | 1.6 |
| 136797_Lung cancer (360) | 0.4 |
| 136794_lung NAT (35D) | 1.2 |
| 136818_Lung NAT (377) | 0.5 |
| 136787_lung cancer (356) | 0.1 |
| 136788_lung NAT (357) | 1.1 |
| 136806_Lung cancer (36B) | 0.1 |
| 136807_Lung NAT (36C) | 0.4 |
| 136789_lung cancer (358) | 0.4 |
| 136802_Lung cancer (365) | 1.6 |
| 136803_Lung cancer (368) | 0.5 |
| 136804_Lung cancer (369) | 1.4 |
| 136811_Lung cancer (370) | 64.2 |
| 136810_Lung NAT (36F) | 3.9 |

Column A - Rel. Exp. (%) Ag3562, Run 263525399

TABLE QC
CNS_neurodegeneration_v1.0

| Tissue Name | A | B |
|---|---|---|
| AD 1 Hippo | 47.3 | 54.7 |
| AD 2 Hippo | 30.1 | 31.2 |
| AD 3 Hippo | 9.9 | 14.6 |
| AD 4 Hippo | 10.4 | 11.6 |
| AD 5 Hippo | 57.4 | 74.2 |
| AD 6 Hippo | 100.0 | 100.0 |
| Control 2 Hippo | 22.7 | 27.7 |
| Control 4 Hippo | 15.7 | 20.6 |
| Control (Path) 3 Hippo | 8.2 | 8.3 |
| AD 1 Temporal Ctx | 33.2 | 42.6 |
| AD 2 Temporal Ctx | 28.3 | 28.5 |
| AD 3 Temporal Ctx | 8.3 | 10.4 |
| AD 4 Temporal Ctx | 15.5 | 18.8 |
| AD 5 Inf Temporal Ctx | 55.1 | 83.5 |
| AD 5 Sup Temporal Ctx | 64.6 | 74.2 |
| AD 6 Inf Temporal Ctx | 58.2 | 68.3 |
| AD 6 Sup Temporal Ctx | 59.5 | 70.7 |
| Control 1 Temporal Ctx | 7.6 | 9.2 |
| Control 2 Temporal Ctx | 15.2 | 27.7 |
| Control 3 Temporal Ctx | 14.1 | 12.3 |
| Control 3 Temporal Ctx | 7.7 | 8.3 |
| Control (Path) 1 Temporal Ctx | 24.5 | 29.1 |
| Control (Path) 2 Temporal Ctx | 15.3 | 17.6 |
| Control (Path) 3 Temporal Ctx | 5.0 | 8.5 |
| Control (Path) 4 Temporal Ctx | 18.9 | 20.6 |
| AD 1 Occipital Ctx | 23.0 | 41.5 |
| AD 2 Occipital Ctx (Missing) | 0.0 | 0.0 |
| AD 3 Occipital Ctx | 10.4 | 10.2 |
| AD 4 Occipital Ctx | 13.1 | 8.2 |
| AD 5 Occipital Ctx | 24.5 | 29.3 |
| AD 6 Occipital Ctx | 24.8 | 35.4 |
| Control 1 Occipital Ctx | 4.7 | 5.0 |
| Control 2 Occipital Ctx | 18.0 | 29.9 |
| Control 3 Occipital Ctx | 11.0 | 13.6 |
| Control 4 Occipital Ctx | 7.3 | 9.2 |
| Control (Path) 1 Occipital Ctx | 32.3 | 31.0 |
| Control (Path) 2 Occipital Ctx | 5.2 | 8.0 |
| Control (Path) 3 Occipital Ctx | 7.6 | 5.6 |
| Control (Path) 4 Occipital Ctx | 10.5 | 12.5 |
| Control 1 Parietal Ctx | 6.1 | 8.1 |
| Control 2 Parietal Ctx | 66.0 | 67.8 |
| Control 3 Parietal Ctx | 19.1 | 18.3 |
| Control (Path) 1 Parietal Ctx | 29.9 | 35.6 |
| Control (Path) 2 Parietal Ctx | 11.5 | 13.0 |
| Control (Path) 3 Parietal Ctx | 5.9 | 8.1 |
| Control (Path) 4 Parietal Ctx | 23.2 | 28.1 |

Column A - Rel. Exp. (%) Ag3562, Run 210629741
Column B - Rel. Exp. (%) Ag3562, Run 224078542

TABLE QD
General_screening_panel_v1.4

| Tissue Name | A |
|---|---|
| Adipose | 2.1 |
| Melanoma* Hs688(A).T | 0.2 |
| Melanoma* Hs688(B).T | 0.3 |
| Melanoma* M14 | 0.0 |
| Melanoma* LOXIMVI | 0.0 |
| Melanoma* SK-MEL-5 | 0.0 |
| Squamous cell carcinoma SCC-4 | 0.5 |
| Testis Pool | 0.2 |
| Prostate ca.* (bone met) PC-3 | 0.0 |
| Prostate Pool | 0.1 |
| Placenta | 0.0 |
| Uterus Pool | 0.1 |
| Ovarian ca. OVCAR-3 | 0.0 |
| Ovarian ca. SK-OV-3 | 11.8 |
| Ovarian ca. OVCAR-4 | 0.0 |
| Ovarian ca. OVCAR-5 | 0.3 |
| Ovarian ca. IGROV-1 | 0.5 |
| Ovarian ca. OVCAR-8 | 0.2 |
| Ovary | 0.2 |
| Breast ca. MCF-7 | 0.5 |
| Breast ca. MDA-MB-231 | 1.1 |
| Breast ca. BT 549 | 1.2 |
| Breast ca. T47D | 0.6 |
| Breast ca. MDA-N | 0.0 |
| Breast Pool | 0.1 |
| Trachea | 1.3 |
| Lung | 0.2 |
| Fetal Lung | 0.3 |
| Lung ca. NCI-N417 | 0.0 |
| Lung ca. LX-1 | 1.6 |
| Lung ca. NCI-H146 | 1.0 |
| Lung ca. SHP-77 | 14.7 |
| Lung ca. A549 | 100.0 |
| Lung ca. NCI-H526 | 0.0 |
| Lung ca. NCI-H23 | 0.2 |
| Lung ca. NCI-H460 | 11.1 |
| Lung ca. HOP-62 | 0.1 |
| Lung ca. NCI-H522 | 0.6 |
| Liver | 0.7 |
| Fetal Liver | 4.1 |
| Liver ca. HepG2 | 2.4 |
| Kidney Pool | 0.2 |
| Fetal Kidney | 0.1 |
| Renal ca. 786-0 | 0.3 |
| Renal ca. A498 | 11.2 |
| Renal ca. ACHN | 0.2 |
| Renal ca. UO-31 | 0.1 |
| Renal ca. TK-10 | 1.5 |
| Bladder | 0.8 |
| Gastric ca. (liver met.) NCI-N87 | 0.1 |
| Gastric ca. KATO III | 0.4 |
| Colon ca. SW-948 | 0.6 |
| Colon ca. SW480 | 0.1 |
| Colon ca.* (SW480 met) SW620 | 0.5 |
| Colon ca. HT29 | 0.5 |
| Colon ca. HCT-116 | 0.0 |
| Colon ca. CaCo-2 | 2.6 |
| Colon cancer tissue | 0.4 |
| Colon ca. SW1116 | 0.0 |
| Colon ca. Colo-205 | 3.0 |
| Colon ca. SW-48 | 0.9 |
| Colon Pool | 0.1 |
| Small Intestine Pool | 0.1 |
| Stomach Pool | 0.1 |
| Bone Marrow Pool | 0.1 |
| Fetal Heart | 0.0 |
| Heart Pool | 0.1 |
| Lymph Node Pool | 0.2 |
| Fetal Skeletal Muscle | 0.1 |
| Skeletal Muscle Pool | 0.7 |
| Spleen Pool | 0.0 |
| Thymus Pool | 0.2 |
| CNS cancer (glio/astro) U87-MG | 0.9 |
| CNS cancer (glio/astro) U-118-MG | 1.0 |
| CNS cancer (neuro; met) SK-N-AS | 0.3 |
| CNS cancer (astro) SF-539 | 0.1 |
| CNS cancer (astro) SNB-75 | 10.2 |
| CNS cancer (glio) SNB-19 | 0.4 |
| CNS cancer (glio) SF-295 | 4.0 |
| Brain (Amygdala) Pool | 0.2 |
| Brain (cerebellum) | 0.1 |
| Brain (fetal) | 0.5 |
| Brain (Hippocampus) Pool | 0.1 |
| Cerebral Cortex Pool | 0.1 |
| Brain (Substantia nigra) Pool | 0.2 |
| Brain (Thalamus) Pool | 0.2 |
| Brain (whole) | 0.4 |
| Spinal Cord Pool | 0.3 |
| Adrenal Gland | 0.2 |
| Pituitary gland Pool | 0.0 |
| Salivary Gland | 0.1 |
| Thyroid (female) | 0.1 |
| Pancreatic ca. CAPAN2 | 0.2 |
| Pancreas Pool | 0.2 |

Column A - Rel. Exp. (%) Ag3562, Run 217240778

TABLE QE
General_screening_panel_v1.6

| Tissue Name | A |
|---|---|
| Adipose | 0.3 |
| Melanoma* Hs688(A).T | 0.3 |
| Melanoma* Hs688(B).T | 0.3 |
| Melanoma* M14 | 0.0 |
| Melanoma* LOXIMVI | 0.0 |
| Melanoma* SK-MEL-5 | 0.0 |
| Squamous cell carcinoma SCC-4 | 0.5 |
| Testis Pool | 0.2 |
| Prostate ca.* (bone met) PC-3 | 0.0 |
| Prostate Pool | 0.1 |
| Placenta | 0.0 |
| Uterus Pool | 0.1 |
| Ovarian ca. OVCAR-3 | 0.0 |
| Ovarian ca. SK-OV-3 | 14.7 |
| Ovarian ca. OVCAR-4 | 0.0 |
| Ovarian ca. OVCAR-5 | 0.3 |
| Ovarian ca. IGROV-1 | 0.4 |
| Ovarian ca. OVCAR-8 | 0.2 |
| Ovary | 0.2 |
| Breast ca. MCF-7 | 0.4 |
| Breast ca. MDA-MB-231 | 1.1 |
| Breast ca. BT 549 | 1.0 |
| Breast ca. T47D | 0.0 |
| Breast ca. MDA-N | 0.0 |
| Breast Pool | 0.1 |
| Trachea | 1.3 |
| Lung | 0.1 |
| Fetal Lung | 0.3 |
| Lung ca. NCI-N417 | 0.0 |
| Lung ca. LX-1 | 1.6 |
| Lung ca. NCI-H146 | 0.8 |
| Lung ca. SHP-77 | 13.9 |
| Lung ca. A549 | 100.0 |
| Lung ca. NCI-H526 | 0.0 |
| Lung ca. NCI-H23 | 0.2 |
| Lung ca. NCI-H460 | 13.7 |
| Lung ca. HOP-62 | 0.0 |
| Lung ca. NCI-H522 | 0.7 |
| Liver | 0.6 |
| Fetal Liver | 4.6 |
| Liver ca. HepG2 | 1.9 |
| Kidney Pool | 0.2 |
| Fetal Kidney | 0.1 |
| Renal ca. 786-0 | 0.3 |
| Renal ca. A498 | 11.3 |
| Renal ca. ACHN | 0.2 |
| Renal ca. UO-31 | 0.1 |
| Renal ca. TK-10 | 1.5 |
| Bladder | 0.8 |
| Gastric ca. (liver met.) NCI-N87 | 0.1 |
| Gastric ca. KATO III | 0.3 |
| Colon ca. SW-948 | 0.5 |
| Colon ca. SW480 | 0.0 |
| Colon ca.* (SW480 met) SW620 | 0.6 |
| Colon ca. HT29 | 0.6 |
| Colon ca. HCT-116 | 0.0 |
| Colon ca. CaCo-2 | 2.4 |
| Colon cancer tissue | 0.4 |
| Colon ca. SW1116 | 0.0 |
| Colon ca. Colo-205 | 4.7 |
| Colon ca. SW-48 | 0.9 |
| Colon Pool | 0.1 |
| Small Intestine Pool | 0.1 |
| Stomach Pool | 0.1 |
| Bone Marrow Pool | 0.0 |
| Fetal Heart | 0.1 |
| Heart Pool | 0.1 |
| Lymph Node Pool | 0.2 |
| Fetal Skeletal Muscle | 0.0 |
| Skeletal Muscle Pool | 0.2 |
| Spleen Pool | 0.0 |
| Thymus Pool | 0.2 |
| CNS cancer (glio/astro) U87-MG | 4.7 |
| CNS cancer (glio/astro) U-118-MG | 1.0 |
| CNS cancer (neuro; met) SK-N-AS | 0.3 |
| CNS cancer (astro) SF-539 | 0.1 |
| CNS cancer (astro) SNB-75 | 9.5 |
| CNS cancer (glio) SNB-19 | 0.4 |
| CNS cancer (glio) SF-295 | 3.4 |
| Brain (Amygdala) Pool | 0.1 |
| Brain (cerebellum) | 0.2 |
| Brain (fetal) | 0.4 |
| Brain (Hippocampus) Pool | 0.1 |
| Cerebral Cortex Pool | 0.1 |
| Brain (Substantia nigra) Pool | 0.1 |
| Brain (Thalamus) Pool | 0.2 |
| Brain (whole) | 0.4 |
| Spinal Cord Pool | 0.4 |
| Adrenal Gland | 0.2 |
| Pituitary gland Pool | 0.0 |
| Salivary Gland | 0.1 |
| Thyroid (female) | 0.1 |
| Pancreatic ca. CAPAN2 | 0.1 |
| Pancreas Pool | 0.1 |

Column A - Rel. Exp. (%) Ag3562, Run 277245085

TABLE QF
HASS Panel v1.0

| Tissue Name | A |
|---|---|
| MCF-7 C1 | 8.5 |
| MCF-7 C2 | 12.0 |
| MCF-7 C3 | 15.7 |
| MCF-7 C4 | 13.2 |
| MCF-7 C5 | 20.2 |
| MCF-7 C6 | 10.4 |
| MCF-7 C7 | 8.8 |
| MCF-7 C9 | 6.9 |
| MCF-7 C10 | 10.2 |
| MCF-7 C11 | 5.8 |
| MCF-7 C12 | 8.7 |
| MCF-7 C13 | 7.6 |
| MCF-7 C15 | 3.6 |
| MCF-7 C16 | 13.2 |
| MCF-7 C17 | 9.8 |
| T24 D1 | 32.8 |
| T24 D2 | 12.2 |
| T24 D3 | 26.4 |
| T24 D4 | 48.3 |
| T24 D5 | 18.2 |
| T24 D6 | 0.2 |
| T24 D7 | 1.0 |
| T24 D9 | 0.0 |
| T24 D10 | 23.7 |
| T24 D11 | 2.7 |
| T24 D12 | 0.1 |
| T24 D13 | 0.3 |
| T24 D15 | 0.3 |
| T24 D16 | 0.2 |
| T24 D17 | 0.5 |
| CAPaN B1 | 4.4 |
| CAPaN B2 | 6.3 |
| CAPaN B3 | 4.3 |
| CAPaN B4 | 3.4 |
| CAPaN B5 | 14.9 |
| CAPaN B6 | 2.1 |
| CAPaN B7 | 1.8 |
| CAPaN B8 | 10.7 |
| CAPaN B9 | 2.6 |
| CAPaN B10 | 6.4 |
| CAPaN B11 | 16.3 |
| CAPaN B12 | 1.7 |
| CAPaN B13 | 1.3 |
| CAPaN B14 | 7.4 |
| CAPaN B15 | 1.2 |
| CAPaN B16 | 4.2 |
| CAPaN B17 | 4.2 |
| U87-MG F1 (B) | 19.6 |
| U87-MG F2 | 5.2 |
| U87-MG F3 | 15.8 |
| U87-MG F4 | 10.2 |
| U87-MG F5 | 40.3 |
| U87-MG F6 | 92.7 |
| U87-MG F7 | 5.9 |
| U87-MG F8 | 9.1 |
| U87-MG F9 | 1.9 |
| U87-MG F10 | 57.4 |
| U87-MG F11 | 100.0 |
| U87-MG F12 | 30.4 |
| U87-MG F13 | 10.2 |
| U87-MG F14 | 16.7 |
| U87-MG F15 | 9.5 |
| U87-MG F16 | 66.4 |
| U87-MG F17 | 67.4 |
| LnCAP A1 | 0.2 |
| LnCAP A2 | 0.2 |
| LnCAP A3 | 0.1 |
| LnCAP A4 | 0.1 |
| LnCAP A5 | 0.2 |
| LnCAP A6 | 0.2 |
| LnCAP A7 | 0.4 |
| LnCAP A8 | 0.6 |
| LnCAP A9 | 0.8 |
| LnCAP A10 | 0.2 |
| LnCAP A11 | 0.4 |
| LnCAP A12 | 0.1 |
| LnCAP A13 | 0.0 |
| LnCAP A14 | 0.2 |
| LnCAP A15 | 0.3 |
| LnCAP A16 | 0.2 |
| LnCAP A17 | 0.5 |
| Primary Astrocytes | 0.4 |
| Primary Renal Proximal Tubule Epithelial cell A2 | 25.0 |
| Primary melanocytes A5 | 1.7 |
| 126443 - 341 medullo | 0.1 |
| 126444 - 487 medullo | 0.0 |
| 126445 - 425 medullo | 0.0 |
| 126446 - 690 medullo | 0.2 |
| 126447 - 54 adult glioma | 3.8 |
| 126448 - 245 adult glioma | 0.0 |
| 126449 - 317 adult glioma | 0.0 |
| 126450 - 212 glioma | 0.1 |
| 126451 - 456 glioma | 0.0 |

Column A - Rel. Exp. (%) Ag3562, Run 276044499

TABLE QG
Oncology_cell_line_screening_panel_v3.1

| Tissue Name | A |
|---|---|
| Daoy Medulloblastoma/Cerebellum | 0.0 |
| TE671 Medulloblastom/Cerebellum | 0.2 |
| D283 Med Medulloblastoma/Cerebellum | 0.0 |
| PFSK-1 Primitive Neuroectodermal/Cerebellum | 0.1 |
| XF-498_CNS | 0.0 |
| SNB-78_CNS/glioma | 0.3 |
| SF-268_CNS/glioblastoma | 0.0 |
| T98G_Glioblastoma | 33.2 |
| SK-N-SH_Neuroblastoma (metastasis) | 0.0 |
| SF-295_CNS/glioblastoma | 2.3 |
| Cerebellum | 0.4 |
| Cerebellum | 0.1 |
| NCI-H292_Mucoepidermoid lung ca. | 0.3 |
| DMS-114_Small cell lung cancer | 0.0 |
| DMS-79_Small cell lung cancer/neuroendocrine | 0.0 |
| NCI-H146_Small cell lung cancer/neuroendocrine | 2.2 |
| NCI-H526_Small cell lung cancer/neuroendocrine | 0.0 |
| NCI-N417_Small cell lung cancer/neuroendocrine | 0.0 |
| NCI-H82_Small cell lung cancer/neuroendocrine | 0.0 |
| NCI-H157_Squamous cell lung cancer (metastasis) | 0.0 |
| NCI-H1155_Large cell lung cancer/neuroendocrine | 0.0 |
| NCI-H1299_Large cell lung cancer/neuroendocrine | 0.0 |
| NCI-H727_Lung carcinoid | 1.3 |
| NCI-UMC-11_Lung carcinoid | 100.0 |
| LX-1_Small cell lung cancer | 1.6 |
| Colo-205_Colon cancer | 7.5 |
| KM12_Colon cancer | 0.1 |
| KM20L2_Colon cancer | 0.1 |
| NCI-H716_Colon cancer | 19.2 |
| SW-48_Colon adenocarcinoma | 2.3 |
| SW1116_Colon adenocarcinoma | 0.0 |
| LS 174T_Colon adenocarcinoma | 0.9 |
| SW-948_Colon adenocarcinoma | 0.3 |
| SW-480_Colon adenocarcinoma | 0.5 |
| NCI-SNU-5_Gastric ca. | 0.0 |
| KATO III_Stomach | 0.1 |
| NCI-SNU-16_Gastric ca. | 2.7 |
| NCI-SNU-1_Gastric ca. | 16.8 |
| RF-1_Gastric adenocarcinoma | 0.5 |
| RF-48_Gastric adenocarcinoma | 0.0 |
| MKN-45_Gastric ca. | 1.6 |
| NCI-N87_Gastric ca. | 0.8 |
| OVCAR-5_Ovarian ca. | 0.4 |
| RL95-2_Uterine carcinoma | 0.5 |
| HelaS3_Cervical adenocarcinoma | 0.9 |
| Ca Ski_Cervical epidermoid carcinoma (metastasis) | 0.3 |
| ES-2_Ovarian clear cell carcinoma | 0.0 |
| Ramos/6 h stim_Stimulated with PMA/ionomycin 6 h | 0.0 |
| Ramos/14 h stim_Stimulated with PMA/ionomycin 14 h | 0.0 |
| MEG-01_Chronic myelogenous leukemia (megokaryoblast) | 0.1 |
| Raji_Burkitt's lymphoma | 0.0 |
| Daudi_Burkitt's lymphoma | 0.0 |
| U266_B-cell plasmacytoma/myeloma | 0.0 |
| CA46_Burkitt's lymphoma | 0.0 |
| RL_non-Hodgkin's B-cell lymphoma | 0.0 |
| JM1_pre-B-cell lymphoma/leukemia | 0.0 |
| Jurkat_T cell leukemia | 0.0 |
| TF-1_Erythroleukemia | 1.6 |
| HUT 78_T-cell lymphoma | 0.0 |
| U937_Histiocytic lymphoma | 0.0 |
| KU-812_Myelogenous leukemia | 0.9 |
| 769-P_Clear cell renal ca. | 1.0 |
| Caki-2_Clear cell renal ca. | 63.7 |
| SW 839_Clear cell renal ca. | 0.4 |
| G401_Wilms' tumor | 0.0 |
| Hs766T_Pancreatic ca. (LN metastasis) | 0.2 |
| CAPAN-1_Pancreatic adenocarcinoma (liver metastasis) | 0.3 |
| SU86.86_Pancreatic carcinoma (liver metastasis) | 1.2 |
| BxPC-3_Pancreatic adenocarcinoma | 3.6 |
| HPAC_Pancreatic adenocarcinoma | 3.1 |
| MIA PaCa-2_Pancreatic ca. | 0.0 |
| CFPAC-1_Pancreatic ductal adenocarcinoma | 3.9 |
| PANC-1_Pancreatic epithelioid ductal ca. | 0.0 |
| T24_Bladder ca. (transitional cell) | 2.0 |
| 5637_Bladder ca. | 0.1 |
| HT-1197_Bladder ca. | 0.0 |
| UM-UC-3_Bladder ca. (transitional cell) | 0.1 |
| A204_Rhabdomyosarcoma | 0.8 |
| HT-1080_Fibrosarcoma | 0.0 |
| MG-63_Osteosarcoma (bone) | 0.0 |
| SK-LMS-1_Leiomyosarcoma (vulva) | 0.0 |
| SJRH30_Rhabdomyosarcoma (met to bone marrow) | 0.1 |
| A431_Epidermoid ca. | 3.8 |
| WM266-4_Melanoma | 0.1 |
| DU 145_Prostate | 0.1 |
| MDA-MB-468_Breast adenocarcinoma | 0.5 |
| SSC-4_Tongue | 0.4 |
| SSC-9_Tongue | 0.2 |
| SSC-15_Tongue | 0.3 |
| CAL 27_Squamous cell ca. of tongue | 0.1 |

Column A - Rel. Exp. (%) Ag3562, Run 222546381

TABLE QH
Panel 2D

| Tissue Name | A |
|---|---|
| Normal Colon | 5.0 |
| CC Well to Mod Diff (ODO3866) | 1.5 |
| CC Margin (ODO3866) | 2.0 |
| CC Gr.2 rectosigmoid (ODO3868) | 0.5 |
| CC Margin (ODO3868) | 0.5 |
| CC Mod Diff (ODO3920) | 0.5 |
| CC Margin (ODO3920) | 1.2 |
| CC Gr.2 ascend colon (ODO3921) | 2.1 |
| CC Margin (ODO3921) | 1.4 |
| CC from Partial Hepatectomy (ODO4309) Mets | 5.5 |
| Liver Margin (ODO4309) | 27.0 |
| Colon mets to lung (OD04451-01) | 0.4 |
| Lung Margin (OD04451-02) | 1.8 |
| Normal Prostate 6546-1 | 1.3 |
| Prostate Cancer (OD04410) | 0.4 |
| Prostate Margin (OD04410) | 0.5 |
| Prostate Cancer (OD04720-01) | 0.6 |
| Prostate Margin (OD04720-02) | 1.6 |
| Normal Lung 061010 | 4.1 |
| Lung Met to Muscle (ODO4286) | 41.8 |
| Muscle Margin (ODO4286) | 1.7 |
| Lung Malignant Cancer (OD03126) | 2.0 |
| Lung Margin (OD03126) | 2.1 |
| Lung Cancer (OD04404) | 100.0 |
| Lung Margin (OD04404) | 1.7 |
| Lung Cancer (OD04565) | 43.2 |
| Lung Margin (OD04565) | 0.7 |
| Lung Cancer (OD04237-01) | 0.5 |
| Lung Margin (OD04237-02) | 2.2 |
| Ocular Mel Met to Liver (ODO4310) | 0.1 |
| Liver Margin (ODO4310) | 14.8 |
| Melanoma Mets to Lung (OD04321) | 0.1 |
| Lung Margin (OD04321) | 2.3 |
| Normal Kidney | 5.0 |
| Kidney Ca, Nuclear grade 2 (OD04338) | 29.1 |
| Kidney Margin (OD04338) | 3.1 |
| Kidney Ca Nuclear grade 1/2 (OD04339) | 3.7 |
| Kidney Margin (OD04339) | 5.1 |
| Kidney Ca, Clear cell type (OD04340) | 6.3 |
| Kidney Margin (OD04340) | 3.6 |
| Kidney Ca, Nuclear grade 3 (OD04348) | 0.1 |
| Kidney Margin (OD04348) | 1.8 |
| Kidney Cancer (OD04622-01) | 1.9 |
| Kidney Margin (OD04622-03) | 1.5 |
| Kidney Cancer (OD04450-01) | 12.0 |
| Kidney Margin (OD04450-03) | 4.5 |
| Kidney Cancer 8120607 | 34.2 |
| Kidney Margin 8120608 | 2.3 |
| Kidney Cancer 8120613 | 4.4 |
| Kidney Margin 8120614 | 4.2 |
| Kidney Cancer 9010320 | 1.5 |
| Kidney Margin 9010321 | 2.6 |
| Normal Uterus | 0.4 |
| Uterus Cancer 064011 | 0.5 |
| Normal Thyroid | 1.2 |
| Thyroid Cancer 064010 | 0.1 |
| Thyroid Cancer A302152 | 0.2 |
| Thyroid Margin A302153 | 1.6 |
| Normal Breast | 5.3 |
| Breast Cancer (OD04566) | 0.2 |
| Breast Cancer (OD04590-01) | 1.4 |
| Breast Cancer Mets (OD04590-03) | 5.2 |
| Breast Cancer Metastasis (OD04655-05) | 1.1 |
| Breast Cancer 064006 | 0.3 |
| Breast Cancer 1024 | 0.8 |
| Breast Cancer 9100266 | 0.3 |
| Breast Margin 9100265 | 0.5 |
| Breast Cancer A209073 | 0.6 |
| Breast Margin A209073 | 0.5 |
| Normal Liver | 11.9 |
| Liver Cancer 064003 | 5.0 |
| Liver Cancer 1025 | 14.7 |
| Liver Cancer 1026 | 3.9 |
| Liver Cancer 6004-T | 14.0 |
| Liver Tissue 6004-N | 20.9 |
| Liver Cancer 6005-T | 3.7 |
| Liver Tissue 6005-N | 6.3 |
| Normal Bladder | 5.6 |
| Bladder Cancer 1023 | 0.3 |
| Bladder Cancer A302173 | 0.8 |
| Bladder Cancer (OD04718-01) | 0.1 |
| Bladder Normal Adjacent (OD04718-03) | 0.9 |
| Normal Ovary | 0.4 |
| Ovarian Cancer 064008 | 0.5 |
| Ovarian Cancer (OD04768-07) | 0.6 |
| Ovary Margin (OD04768-08) | 0.3 |
| Normal Stomach | 5.5 |
| Gastric Cancer 9060358 | 1.0 |
| Stomach Margin 9060359 | 11.7 |
| Gastric Cancer 9060395 | 14.4 |
| Stomach Margin 9060394 | 15.3 |
| Gastric Cancer 9060397 | 2.1 |
| Stomach Margin 9060396 | 6.7 |
| Gastric Cancer 064005 | 7.6 |

Column A - Rel. Exp. (%) Ag3562, Run 170858350

TABLE RI
Panel 4.1D

| Tissue Name | A |
|---|---|
| Secondary Th1 act | 0.0 |
| Secondary Th2 act | 0.1 |
| Secondary Tr1 act | 0.2 |
| Secondary Th1 rest | 0.2 |
| Secondary Th2 rest | 0.1 |
| Secondary Tr1 rest | 0.1 |
| Primary Th1 act | 0.0 |
| Primary Th2 act | 0.1 |
| Primary Tr1 act | 0.0 |
| Primary Th1 rest | 0.4 |
| Primary Th2 rest | 0.2 |
| Primary Tr1 rest | 0.1 |
| CD45RA CD4 lymphocyte act | 6.7 |
| CD45RO CD4 lymphocyte act | 0.4 |
| CD8 lymphocyte act | 0.3 |
| Secondary CD8 lymphocyte rest | 0.0 |
| Secondary CD8 lymphocyte act | 0.0 |
| CD4 lymphocyte none | 0.6 |
| 2ry Th1/Th2/Tr1_anti-CD95 CH11 | 0.2 |
| LAK cells rest | 0.5 |
| LAK cells IL-2 | 1.3 |
| LAK cells IL-2 + IL-12 | 0.5 |
| LAK cells IL-2 + IFN gamma | 1.1 |
| LAK cells IL-2 + IL-18 | 0.7 |
| LAK cells PMA/ionomycin | 1.2 |
| NK Cells IL-2 rest | 3.5 |
| Two Way MLR 3 day | 1.4 |
| Two Way MLR 5 day | 0.6 |
| Two Way MLR 7 day | 0.3 |
| PBMC rest | 0.8 |
| PBMC PWM | 15.0 |
| PBMC PHA-L | 0.3 |
| Ramos (B cell) none | 0.0 |
| Ramos (B cell) ionomycin | 0.0 |
| B lymphocytes PWM | 0.2 |
| B lymphocytes CD40L and IL-4 | 0.3 |
| EOL-1 dbcAMP | 0.1 |
| EOL-1 dbcAMP PMA/ionomycin | 0.1 |
| Dendritic cells none | 0.8 |
| Dendritic cells LPS | 2.9 |
| Dendritic cells anti-CD40 | 0.6 |
| Monocytes rest | 0.1 |
| Monocytes LPS | 9.7 |
| Macrophages rest | 2.4 |
| Macrophages LPS | 2.9 |
| HUVEC none | 1.0 |
| HUVEC starved | 0.9 |
| HUVEC IL-1beta | 0.7 |
| HUVEC IFN gamma | 2.1 |
| HUVEC TNF alpha + IFN gamma | 0.7 |
| HUVEC TNF alpha + IL4 | 0.5 |
| HUVEC IL-11 | 2.4 |
| Lung Microvascular EC none | 13.1 |
| Lung Microvascular EC TNFalpha + IL-1beta | 10.1 |
| Microvascular Dermal EC none | 11.8 |
| Microsvasular Dermal EC TNFalpha + IL-1beta | 12.8 |
| Bronchial epithelium TNFalpha + IL1beta | 92.7 |
| Small airway epithelium none | 29.9 |
| Small airway epithelium TNFalpha + IL-1beta | 50.3 |
| Coronery artery SMC rest | 5.4 |
| Coronery artery SMC TNFalpha + IL-1beta | 7.0 |
| Astrocytes rest | 0.6 |
| Astrocytes TNFalpha + IL-1beta | 0.7 |
| KU-812 (Basophil) rest | 16.4 |
| KU-812 (Basophil) PMA/ionomycin | 33.2 |
| CCD1106 (Keratinocytes) none | 2.9 |
| CCD1106 (Keratinocytes) TNFalpha + IL-1beta | 2.3 |
| Liver cirrhosis | 38.2 |
| NCI-H292 none | 11.8 |
| NCI-H292 IL-4 | 7.1 |
| NCI-H292 IL-9 | 22.5 |
| NCI-H292 IL-13 | 5.2 |
| NCI-H292 IFN gamma | 4.7 |
| HPAEC none | 5.1 |
| HPAEC TNF alpha + IL-1 beta | 7.6 |
| Lung fibroblast none | 6.8 |
| Lung fibroblast TNF alpha + IL-1 beta | 48.6 |
| Lung fibroblast IL-4 | 7.5 |
| Lung fibroblast IL-9 | 7.2 |
| Lung fibroblast IL-13 | 8.8 |
| Lung fibroblast IFN gamma | 6.8 |
| Dermal fibroblast CCD1070 rest | 13.9 |
| Dermal fibroblast CCD1070 TNF alpha | 19.9 |
| Dermal fibroblast CCD1070 IL-1 beta | 24.7 |
| Dermal fibroblast IFN gamma | 38.4 |
| Dermal fibroblast IL-4 | 100.0 |
| Dermal Fibroblasts rest | 68.3 |
| Neutrophils TNFa + LPS | 0.2 |
| Neutrophils rest | 0.1 |
| Colon | 14.0 |
| Lung | 5.8 |
| Thymus | 6.6 |
| Kidney | 20.7 |

Column A - Rel. Exp. (%) Ag3562, Run 169990867

TABLE QJ
Panel 5 Islet

| Tissue Name | A |
|---|---|
| 97457_Patient-02go_adipose | 12.8 |
| 97476_Patient-07sk_skeletal muscle | 12.4 |
| 97477_Patient-07ut_uterus | 1.6 |
| 97478_Patient-07pl_placenta | 0.3 |
| 99167_Bayer Patient 1 | 100.0 |
| 97482_Patient-08ut_uterus | 0.7 |
| 97483_Patient-08pl_placenta | 0.0 |
| 97486_Patient-09sk_skeletal muscle | 7.6 |
| 97487_Patient-09ut_uterus | 3.9 |
| 97488_Patient-09pl_placenta | 0.4 |
| 97492_Patient-10ut_uterus | 1.9 |
| 97493_Patient-10pl_placenta | 0.5 |
| 97495_Patient-11go_adipose | 7.4 |
| 97496_Patient-11sk_skeletal muscle | 8.0 |
| 97497_Patient-11ut_uterus | 2.8 |
| 97498_Patient-11pl_placenta | 0.1 |
| 97500_Patient-12go_adipose | 11.3 |
| 97501_Patient-12sk_skeletal muscle | 33.2 |
| 97502_Patient-12ut_uterus | 3.1 |
| 97503_Patient-12pl_placenta | 0.4 |
| 94721_Donor 2 U - A_Mesenchymal Stem Cells | 3.8 |
| 94722_Donor 2 U - B_Mesenchymal Stem Cells | 4.3 |
| 94723_Donor 2 U - C_Mesenchymal Stem Cells | 6.0 |
| 94709_Donor 2 AM - A_adipose | 10.2 |
| 94710_Donor 2 AM - B_adipose | 7.1 |
| 94711_Donor 2 AM - C_adipose | 6.4 |
| 94712_Donor 2 AD - A_adipose | 17.9 |
| 94713_Donor 2 AD - B_adipose | 15.6 |
| 94714_Donor 2 AD - C_adipose | 19.1 |
| 94742_Donor 3 U - A_Mesenchymal Stem Cells | 2.0 |
| 94743_Donor 3 U - B_Mesenchymal Stem Cells | 3.4 |
| 94730_Donor 3 AM - A_adipose | 17.6 |
| 94731_Donor 3 AM - B_adipose | 7.6 |
| 94732_Donor 3 AM - C_adipose | 9.7 |
| 94733_Donor 3 AD - A_adipose | 32.5 |
| 94734_Donor 3 AD - B_adipose | 6.7 |
| 94735_Donor 3 AD - C_adipose | 23.2 |
| 77138_Liver_HepG2untreated | 82.9 |
| 73556_Heart_Cardiac stromal cells (primary) | 1.9 |
| 81735_Small Intestine | 13.4 |
| 72409_Kidney_Proximal Convoluted Tubule | 3.6 |
| 82685_Small intestine_Duodenum | 2.5 |
| 90650_Adrenal_Adrenocortical adenoma | 2.4 |
| 72410_Kidney_HRCE | 11.6 |
| 72411_Kidney_HRE | 1.3 |
| 73139_Uterus_Uterine smooth muscle cells | 0.4 |

Column A - Rel. Exp. (%) Ag3562, Run 242386397

Ardais Panel v.1.0 Summary: Ag3562 Highest expression of this gene is seen in lung cancer (CT=19.1). In addition, this gene is more highly expressed in three lung cancer samples than in the corresponding normal adjacent tissue. Thus, expression of this gene could be used as a marker of this cancer. Furthemore, therapeutic modulation of the expression or function of this gene product may be useful in the treatment of lung cancer.

CNS_neurodegeneration_v1.0 Summary: Data obtained from RTQPCR assays performed for this panel was analyized using multivariate analysis (ANOVA). The multivariate results obtained analyzing Ag3562 in this panel confirms the expression of the CG59693-02 gene at low levels in the brain in an independent group of individuals. This gene is found to be upregulated in the temporal cortex of Alzheimer's disease patients when analyzed by ANCOVA, (p=0.002). Therefore, modulation of gene expression with small molecule therapeutic or biomolecules may prevent or slow the progression of Alzheimer's disease.

General_screening_panel_v1.4 Summary: Ag3562 Highest expression of the CG59693-02 gene is detected in lung cancer A549 cell line (CT=20.01). High expression of this gene is also seen in cluster of cancer cell lines derived from gastric, colon, lung, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of gastric, colon, lung, renal, breast, ovarian, prostate, squamous cell carcinoma, melanoma and brain cancers.

Among tissues with metabolic or endocrine function, this gene is expressed at moderate to high levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.6 Summary: Ag3562 Highest expression of the CG59693-02 gene is detected in lung cancer A549 cell line (CT=20.7). High expression of this gene is also seen in cluster of cancer cell lines derived from gastric, colon, lung, renal, breast, ovarian, prostate, squamous cell carcinoma, melanoma and brain cancers. Among tissues with metabolic or endocrine function, this gene is expressed at moderate to high levels in pancreas, adipose, adrenal gland, thyroid, pituitary gland, skeletal muscle, heart, liver and the gastrointestinal tract. In addition, this gene is expressed at high levels in all regions of the central nervous system examined, including amygdala, hippocampus, substantia nigra, thalamus, cerebellum, cerebral cortex, and spinal cord. This pattern is in agreement with the expression profile in General_screening_panel_v1.4. Please see panel 1.4 for a discussion of this gene

HASS Panel v1.0 Summary: Ag3562. The expression of CG59693-02 gene is not increased by oxygen deprivation, acidic or a serum starved environment in the breast, bladder, pancreatic and prostate cell line in this panel.

However expression is increased in a glioblastoma/astrocytoma cell line when these cells are subjected to an acidic environment (Maximum expression U87-MG F21; CT=23.96) which suggests that expression may also be upregulated in the acidic regions of brain cancers. Moderate to low expression is also shown in 2 of 5 glioma and 2 of 4 medulloblastoma tissue samples in this panel. Therapeutic modulation of this gene product using small molecule drugs may be useful in the treatment of brain cancer.

Oncology_cell_line_screening_panel_v3.1 Summary: Ag3562 Highest expression of the CG59693-02 gene is detected in lung carcinoid sample (CT=21.7). High to moderate levels of expression of this gene is also seen in number of cancer samples including tongue, breast, prostate, melanoma, bone marrow, bladder, pancreatic, renal, lymphoma, ovarian, cervical, uterine, gastric, lung and brain cancer. Therefore, therapeutic modulation of this gene through the use of small molecule drug may be beneficial in the treatment of these cancers.

Panel 2D Summary: Ag3562 Highest expression of the CG59693-02 gene is detected in lung cancer (CT=23.5). High expression of this gene is seen in number of lung cancer samples. Expression of this gene is higher in cancer sample as compared to corresponding adjacent control samples. Therefore, expression of this gene may be used as marker to detect the presence of lung cancer and therapeutic modulation of this gene through the use of small molecule drug may be useful in the treatment of lung cancer.

High to moderate levels of expression of this gene is also seen in number of cancer samples including colon, gastric, ovarian, liver, breast, thyroid, kidney, and prostate cancers. Therefore, therapeutic modulation of this gene through the use of small molecule drug may be beneficial in the treatment of these cancers.

Panel 4.1D Summary: Ag3562 Highest expression of the CG59693-02 gene is detected in IL-4 treated dermal fibroblasts (CT=25.2). This gene is expressed at moderate to low levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag3562 Highest expression of the CG59693-02 gene is detected in islet cells (Bayer patient 1) (CT=25.3). High to moderate levels of expression of this gene is also seen in adipose, skeletal muscle, placenta, uterus, liver, heart, small intestine and kidney. Therefore, therapeutic modulation of the activity of this gene may prove useful in the treatment of endocrine/metabolically related diseases, such as obesity and diabetes.

R. CG93088-01: Moncarboxylate Transporter

Expression of gene CG93088-01 was assessed using the primer-probe set Ag3841, described in Table RA. Results of the RTQ-PCR runs are shown in Tables RB, RC and RD.

TABLE RA

Probe Name Ag3841

| | | | | |
|---|---|---|---|---|
| | | | Start | SEQ ID |
| Primers | Sequences | Length | Position | No |

| | | | | |
|---|---|---|---|---|
| Forward | 5′-ttcctatggcattgttgtaggt-3′ | 22 | 583 | 171 |
| Probe | TET-5′-tggtttattatacactgcaacagtgacca-3′-TAMRA | 29 | 613 | 172 |
| Reverse | 5′-atcgtcaaaatactggcacgta-3′ | 22 | 643 | 173 |

TABLE RB
CNS_neurodegeneration_v1.0

| Tissue Name | A | B |
|---|---|---|
| AD 1 Hippo | 59.5 | 60.7 |
| AD 2 Hippo | 91.4 | 77.4 |
| AD 3 Hippo | 15.8 | 13.0 |
| AD 4 Hippo | 15.0 | 19.3 |
| AD 5 Hippo | 43.2 | 52.5 |
| AD 6 Hippo | 92.7 | 100.0 |
| Control 2 Hippo | 25.5 | 32.3 |
| Control 4 Hippo | 45.7 | 54.7 |
| Control (Path) 3 Hippo | 16.6 | 14.8 |
| AD 1 Temporal Ctx | 55.1 | 54.3 |
| AD 2 Temporal Ctx | 61.6 | 62.0 |
| AD 3 Temporal Ctx | 12.9 | 16.4 |
| AD 4 Temporal Ctx | 42.9 | 44.1 |
| AD 5 Inf Temporal Ctx | 96.6 | 92.0 |
| AD 5 Sup Temporal Ctx | 100.0 | 83.5 |
| AD 6 Inf Temporal Ctx | 51.4 | 48.3 |
| AD 6 Sup Temporal Ctx | 56.3 | 49.0 |
| Control 1 Temporal Ctx | 18.4 | 15.9 |
| Control 2 Temporal Ctx | 23.0 | 27.4 |
| Control 3 Temporal Ctx | 12.4 | 17.9 |
| Control 3 Temporal Ctx | 19.9 | 25.5 |
| Control (Path) 1 Temporal Ctx | 22.2 | 20.7 |
| Control (Path) 2 Temporal Ctx | 29.1 | 26.6 |
| Control (Path) 3 Temporal Ctx | 11.8 | 9.9 |
| Control (Path) 4 Temporal Ctx | 26.4 | 19.1 |
| AD 1 Occipital Ctx | 35.1 | 39.0 |
| AD 2 Occipital Ctx (Missing) | 0.0 | 0.0 |
| AD 3 Occipital Ctx | 16.8 | 16.0 |
| AD 4 Occipital Ctx | 31.9 | 28.1 |
| AD 5 Occipital Ctx | 23.8 | 13.4 |
| AD 6 Occipital Ctx | 15.2 | 28.1 |
| Control 1 Occipital Ctx | 7.0 | 6.1 |
| Control 2 Occipital Ctx | 35.4 | 32.3 |
| Control 3 Occipital Ctx | 20.6 | 18.0 |
| Control 4 Occipital Ctx | 16.8 | 22.7 |
| Control (Path) 1 Occipital Ctx | 52.5 | 50.0 |
| Control (Path) 2 Occipital Ctx | 9.2 | 12.2 |
| Control (Path) 3 Occipital Ctx | 5.1 | 5.3 |
| Control (Path) 4 Occipital Ctx | 6.3 | 6.2 |
| Control 1 Parietal Ctx | 14.4 | 15.9 |
| Control 2 Parietal Ctx | 63.7 | 76.3 |
| Control 3 Parietal Ctx | 14.3 | 14.9 |
| Control (Path) 1 Parietal Ctx | 24.0 | 28.3 |
| Control (Path) 2 Parietal Ctx | 25.5 | 24.8 |
| Control (Path) 3 Parietal Ctx | 8.8 | 7.9 |
| Control (Path) 4 Parietal Ctx | 27.7 | 21.2 |

Column A - Rel. Exp. (%) Ag3841, Run 206873281
Column B - Rel. Exp. (%) Ag3841, Run 224339890

TABLE RC
General_screening_panel_v1.4

| Tissue Name | A |
|---|---|
| Adipose | 1.6 |
| Melanoma* Hs688(A).T | 0.0 |
| Melanoma* Hs688(B).T | 0.1 |
| Melanoma* M14 | 0.0 |
| Melanoma* LOXIMVI | 0.0 |
| Melanoma* SK-MEL-5 | 0.4 |
| Squamous cell carcinoma SCC-4 | 5.7 |
| Testis Pool | 2.3 |
| Prostate ca.* (bone met) PC-3 | 10.2 |
| Prostate Pool | 2.3 |
| Placenta | 0.0 |
| Uterus Pool | 3.8 |
| Ovarian ca. OVCAR-3 | 3.0 |
| Ovarian ca. SK-OV-3 | 1.9 |
| Ovarian ca. OVCAR-4 | 1.3 |
| Ovarian ca. OVCAR-5 | 19.9 |
| Ovarian ca. IGROV-1 | 0.4 |
| Ovarian ca. OVCAR-8 | 1.6 |
| Ovary | 30.6 |
| Breast ca. MCF-7 | 2.4 |
| Breast ca. MDA-MB-231 | 0.0 |
| Breast ca. BT 549 | 3.8 |
| Breast ca. T47D | 33.9 |
| Breast ca. MDA-N | 0.0 |
| Breast Pool | 9.7 |
| Trachea | 8.2 |
| Lung | 5.2 |
| Fetal Lung | 3.0 |
| Lung ca. NCI-N417 | 0.7 |
| Lung ca. LX-1 | 0.3 |
| Lung ca. NCI-H146 | 0.5 |
| Lung ca. SHP-77 | 0.0 |
| Lung ca. A549 | 0.1 |
| Lung ca. NCI-H526 | 0.0 |
| Lung ca. NCI-H23 | 3.8 |
| Lung ca. NCI-H460 | 2.0 |
| Lung ca. HOP-62 | 0.2 |
| Lung ca. NCI-H522 | 2.4 |
| Liver | 0.1 |
| Fetal Liver | 8.0 |
| Liver ca. HepG2 | 3.1 |
| Kidney Pool | 8.7 |
| Fetal Kidney | 12.9 |
| Renal ca. 786-0 | 0.1 |
| Renal ca. A498 | 0.5 |
| Renal ca. ACHN | 0.7 |
| Renal ca. UO-31 | 1.8 |
| Renal ca. TK-10 | 5.6 |
| Bladder | 3.1 |
| Gastric ca. (liver met.) NCI-N87 | 2.6 |
| Gastric ca. KATO III | 0.0 |
| Colon ca. SW-948 | 0.8 |
| Colon ca. SW480 | 1.7 |
| Colon ca.* (SW480 met) SW620 | 2.4 |
| Colon ca. HT29 | 0.0 |
| Colon ca. HCT-116 | 8.7 |
| Colon ca. CaCo-2 | 0.6 |
| Colon cancer tissue | 0.2 |
| Colon ca. SW1116 | 0.3 |
| Colon ca. Colo-205 | 0.0 |
| Colon ca. SW-48 | 0.0 |
| Colon Pool | 9.9 |
| Small Intestine Pool | 3.8 |
| Stomach Pool | 2.9 |
| Bone Marrow Pool | 2.7 |
| Fetal Heart | 0.8 |
| Heart Pool | 1.9 |
| Lymph Node Pool | 8.8 |
| Fetal Skeletal Muscle | 1.4 |
| Skeletal Muscle Pool | 0.7 |
| Spleen Pool | 22.4 |
| Thymus Pool | 3.1 |
| CNS cancer (glio/astro) U87-MG | 0.0 |
| CNS cancer (glio/astro) U-118-MG | 0.0 |
| CNS cancer (neuro; met) SK-N-AS | 3.0 |
| CNS cancer (astro) SF-539 | 2.5 |
| CNS cancer (astro) SNB-75 | 2.6 |
| CNS cancer (glio) SNB-19 | 0.5 |
| CNS cancer (glio) SF-295 | 0.4 |
| Brain (Amygdala) Pool | 1.0 |
| Brain (cerebellum) | 5.5 |
| Brain (fetal) | 4.7 |
| Brain (Hippocampus) Pool | 2.9 |
| Cerebral Cortex Pool | 2.1 |
| Brain (Substantia nigra) Pool | 1.8 |
| Brain (Thalamus) Pool | 2.6 |
| Brain (whole) | 3.1 |
| Spinal Cord Pool | 3.8 |
| Adrenal Gland | 100.0 |
| Pituitary gland Pool | 2.1 |
| Salivary Gland | 1.1 |
| Thyroid (female) | 5.2 |
| Pancreatic ca. CAPAN2 | 0.2 |
| Pancreas Pool | 5.2 |

Column A - Rel. Exp. (%) Ag3841, Run 213604526

TABLE RD
Panel 4.1D

| Tissue Name | A |
|---|---|
| Secondary Th1 act | 0.3 |
| Secondary Th2 act | 0.3 |
| Secondary Tr1 act | 0.3 |
| Secondary Th1 rest | 0.0 |
| Secondary Th2 rest | 0.1 |
| Secondary Tr1 rest | 0.0 |
| Primary Th1 act | 2.4 |
| Primary Th2 act | 1.0 |
| Primary Tr1 act | 1.0 |
| Primary Th1 rest | 0.0 |
| Primary Th2 rest | 0.0 |
| Primary Tr1 rest | 0.1 |
| CD45RA CD4 lymphocyte act | 0.2 |
| CD45RO CD4 lymphocyte act | 0.3 |
| CD8 lymphocyte act | 0.2 |
| Secondary CD8 lymphocyte rest | 0.1 |
| Secondary CD8 lymphocyte act | 0.1 |
| CD4 lymphocyte none | 0.0 |
| 2ry Th1/Th2/Tr1_anti-CD95 CH11 | 0.0 |
| LAK cells rest | 0.1 |
| LAK cells IL-2 | 0.1 |
| LAK cells IL-2 + IL-12 | 0.3 |
| LAK cells IL-2 + IFN gamma | 0.2 |
| LAK cells IL-2 + IL-18 | 0.5 |
| LAK cells PMA/ionomycin | 0.0 |
| NK Cells IL-2 rest | 0.1 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.7 |
| PBMC PHA-L | 0.1 |
| Ramos (B cell) none | 0.0 |
| Ramos (B cell) ionomycin | 0.0 |
| B lymphocytes PWM | 0.9 |
| B lymphocytes CD40L and IL-4 | 0.2 |
| EOL-1 dbcAMP | 0.0 |
| EOL-1 dbcAMP PMA/ionomycin | 0.0 |
| Dendritic cells none | 0.0 |
| Dendritic cells LPS | 0.1 |
| Dendritic cells anti-CD40 | 0.2 |
| Monocytes rest | 0.0 |
| Monocytes LPS | 0.0 |
| Macrophages rest | 0.1 |
| Macrophages LPS | 0.0 |
| HUVEC none | 0.0 |
| HUVEC starved | 0.0 |
| HUVEC IL-1beta | 0.2 |
| HUVEC IFN gamma | 1.3 |
| HUVEC TNF alpha + IFN gamma | 0.1 |
| HUVEC TNF alpha + IL4 | 0.3 |
| HUVEC IL-11 | 1.0 |
| Lung Microvascular EC none | 2.3 |
| Lung Microvascular EC TNFalpha + IL-1beta | 1.4 |
| Microvascular Dermal EC none | 0.6 |
| Microsvasular Dermal EC TNFalpha + IL-1beta | 0.1 |
| Bronchial epithelium TNFalpha + IL1beta | 4.9 |
| Small airway epithelium none | 2.9 |
| Small airway epithelium TNFalpha + IL-1beta | 3.1 |
| Coronery artery SMC rest | 3.7 |
| Coronery artery SMC TNFalpha + IL-1beta | 2.3 |
| Astrocytes rest | 4.4 |
| Astrocytes TNFalpha + IL-1beta | 2.9 |
| KU-812 (Basophil) rest | 18.6 |
| KU-812 (Basophil) PMA/ionomycin | 16.4 |
| CCD1106 (Keratinocytes) none | 3.5 |
| CCD1106 (Keratinocytes) TNFalpha + IL-1beta | 2.5 |
| Liver cirrhosis | 0.1 |
| NCI-H292 none | 0.0 |
| NCI-H292 IL-4 | 0.2 |
| NCI-H292 IL-9 | 0.2 |
| NCI-H292 IL-13 | 0.1 |
| NCI-H292 IFN gamma | 0.4 |
| HPAEC none | 0.1 |
| HPAEC TNF alpha + IL-1 beta | 1.5 |
| Lung fibroblast none | 0.1 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.3 |
| Lung | 0.2 |
| Thymus | 0.3 |
| Kidney | 100.0 |

Column A - Rel. Exp. (%) Ag3841, Run 170126778

CNS_neurodegeneration_v1.0 Summary: Data obtained from RTQPCR assays performed for this panel was analyized using multivariate analysis (ANOVA). The multivariate results obtained analyzing Ag3841 shows two experiments tested with the same probe and primer sets are in excellent agreements. It confirms the expression of the CG93088-01 gene at low levels in the brain in an independent group of individuals. This gene is upregulated in the temporal cortex of Alzheimer's disease patients when compared with non-demented controls (p=0.02 when analyzed by Ancova, estimate of total cDNA loaded per well used asa covariate). This gene may therefore be a small molecule target, and blockade of this transporter may slow or stop the progression of Alzheimer's disease.

General_screening_panel_v1.4 Summary: Ag3841 Highest expression of the CG93088-01 gene is detected in adrenal gland (CT=25). In addition, this gene is also expressed at high to moderate levels in other tissues with metabolic or endocrine function, such as pancreas, adipose, thyroid, pituitary gland, skeletal muscle, heart, liver and the gastrointestinal tract. The CG93088-01 gene codes for monocarboxylate transporter, a transporter belonging to sugar transporter family. Recently, a protein belonging to this family was shown to be associated with non-insulin-dependent diabetes mellitus (NIDDM) (Ref.1). Therefore, therapeutic modulation of the activity of this gene may prove useful in the treatment of endocrine/metabolically related diseases, such as obesity and diabetes including NIDDM.

Interestingly, this gene is expressed at much higher levels in fetal (CT=28.7) when compared to adult liver (CT=35.6). This observation suggests that expression of this gene can be used to distinguish disorders or predisposition thereto between fetal and adult liver.

In addition, this gene is expressed at high to moderate levels in all regions of the central nervous system examined, including amygdala, hippocampus, substantia nigra, thalamus, cerebellum, cerebral cortex, and spinal cord. Therefore, this gene may play a role in central nervous system disorders such as Alzheimer's disease, Parkinson's disease, epilepsy, multiple sclerosis, schizophrenia and depression.

References.

McVie-Wylie A J, et al. (2001) Genomics 72(1):113-7 (PMID: 11247674)

Panel 4.1D Summary: Ag3841 Highest expression of the CG93088-01 gene is detected in kidney sample (CT=26). Therefore, antibody or small molecule therapies designed with the protein encoded for by this gene could modulate kidney function and be important in the treatment of inflammatory or autoimmune diseases that affect the kidney, including lupus and glomerulonephritis.

In addition, low to moderate expression of this gene is also seen in TNF alpha+IL-1 beta treated HPAEC, keratinocytes, basophils, astrocytes, coronery artery SMC, small airway epithelium, lung microvascular EC, microvascular dermal EC and PWM treated B lymphocytes. Interestingly, expression of this gene is stimulated in TNF alpha+IL-1 beta treated HPAEC, IFN gamma/IL-11 treated HUVEC cells, PWM treated PBMC cells, IL-2+IL-18 treated LAK cells, activated primary and secondary Th1, Th2, Tr1 cells as compared to their corresponding untreated or resting cells. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

### Example D

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

TABLE D1
NOV2a SNP Data CG180777-01

| | Nucleotides | | | Amino Acids | | |
|---|---|---|---|---|---|---|
| Variant | Position | Initial | Modified | Position | Initial | Modified |
| 13382237 | 566 | A | G | 171 | His | Arg |

TABLE D2
NOV4a SNP Data CG50183-01

| | Nucleotides | | | Amino Acids | | |
|---|---|---|---|---|---|---|
| Variant | Position | Initial | Modified | Position | Initial | Modified |
| 13382220 | 148 | T | C | 49 | Val | Ala |

TABLE D3
NOV10a SNP Data CG56151-01

| | Nucleotides | | | Amino Acids | | |
|---|---|---|---|---|---|---|
| Variant | Position | Initial | Modified | Position | Initial | Modified |
| 13375160 | 77 | T | A | 13 | Thr | Thr |
| 13375159 | 86 | T | C | 16 | Thr | Thr |
| 13375158 | 242 | C | T | 68 | Pro | Pro |
| 13375157 | 367 | C | T | 110 | Thr | Ile |
| 13375156 | 421 | T | C | 128 | Met | Thr |
| 13375155 | 1301 | C | T | 421 | Phe | Phe |
| 13375153 | 1475 | C | T | 479 | Phe | Phe |
| 13375151 | 1526 | T | C | 496 | Ala | Ala |

TABLE D4
NOV11a: SNP Variants for CG56155-02.

| | Nucleotides | | | Amino Acids | | |
|---|---|---|---|---|---|---|
| Variant | Position | Initial | Modified | Position | Initial | Modified |
| 13374617 | 437 | A | G | 143 | Asn | Ser |
| 13375310 | 664 | T | G | 219 | Phe | Val |
| 13382227 | 1150 | G | T | 381 | Ala | Ser |
| 13375308 | 1210 | G | T | 401 | Glu | End |
| 13382226 | 1770 | C | T | 587 | Asn | Asn |

TABLE D5
NOV18a SNP Data CG57758-02

| | Nucleotides | | | Amino Acids | | |
|---|---|---|---|---|---|---|
| Variant | Position | Initial | Modified | Position | Initial | Modified |
| 13376438 | 1466 | T | C | 479 | Val | Ala |
| 13376435 | 1805 | T | C | 592 | Val | Ala |
| 13376434 | 1826 | T | C | 599 | Ile | Thr |

TABLE D6
NOV19a SNP Data CG59693-01

| | Nucleotides | | | Amino Acids | | |
|---|---|---|---|---|---|---|
| Variant | Position | Initial | Modified | Position | Initial | Modified |
| 13375931 | 259 | T | A | 87 | Cys | Ser |
| 13375927 | 357 | A | G | 119 | Pro | Pro |
| 13375928 | 593 | A | G | 198 | Asn | Ser |
| 13375925 | 666 | C | T | 222 | His | His |
| 13375924 | 783 | A | G | 261 | Leu | Leu |

TABLE D7
NOV20a SNP Data CG93088-01

| | Nucleotides | | | Amino Acids | | |
|---|---|---|---|---|---|---|
| Variant | Position | Initial | Modified | Position | Initial | Modified |
| 13377733 | 1458 | C | T | 399 | Ala | Val |

### Example E

### CuraChip Gene Expression

CuraGen has developed a gene microarray (CuraChip 1.2) for target identification. It provides a high-throughput means of global mRNA expression analyses of CuraGen's collection of cDNA sequences representing the Pharmaceutically Tractable Genome (PTG). This sequence set includes genes which can be developed into protein therapeutics, or used to develop antibody or small molecule therapeutics. CuraChip 1.2 contains ˜11,000 oligos representing approximately 8,500 gene loci, including (but not restricted to) kinases, ion channels, G-protein coupled receptors (GPCRs), nuclear hormone receptors, proteases, transporters, metabolic enzymes, hormones, growth factors, chemokines, cytokines, complement and coagulation factors, and cell surface receptors.

The CuraChip cDNAs were represented as 30-mer oligodeoxyribonucleotides (oligos) on a glass microchip. Hybridization methods using the longer CuraChip oligos are more specific compared to methods using 25-mer oligos. CuraChip oligos were synthesized with a linker, purified to remove truncated oligos (which can influence hybridization strength and specificity), and spotted on a glass slide. Oligo-dT primers were used to generate cRNA probes for hybridization from samples of interest. A biotin-avidin conjugation system was used to detect hybridized probes with a fluorophore-labeled secondary antibody. Gene expression was analyzed using clustering and correlation bioinformatics tools such as Spotfire® (Spotfire, Inc., 212 Elm Street, Somerville, Mass. 02144) and statistical tools such as multivariate analysis (MVA).

Normalization Method Used in CuraChip Software

The median fluorescence intensity of each spot and a background for each spot is read on a scale from 0 to 65,000. CuraGen's CuraChip software, developed in-house, has the capability to present the user with either the raw data (median intensities) or normalized data. If normalized data is chosen, the CuraChip software uses the following method to do mean normalization. The normalization is based on each slide/experiment.

fg_median is the signal/foreground median for each slide/experiment;

bg_median is the background median for each slide/experiment;

original_value is the difference between fg_median and bg_median;

flag is an indicator of a spot's success or failure, where 0 means success and 1 means failure;

raw_fg_mean is the raw foreground mean for each slide/experiment;

raw_bg_mean is the raw background mean for each slide/experiment;

trim_percentage is the trim percentage for each slide/experiment; this could be defined by the user; currently we are using 2% as the trim percentage for each slide/experiment;

nSpots is the number of spots on each slide;

nSlides is the number of slides in each experiment;

fg_mean is the trimmed foreground mean for each slide/experiment;

bg_mean is the trimmed background mean for each slide/experiment;

max_fg_mean is a constant among all slides/experiments, currently 2200.0;

normalized_value is the final normalized value;

coeff is the normalization co-efficient;

MAX_VALUE is a constant representing the highest possible fluorescence reading, currently 65,000.

### Step 1

### Calculate Trimmed Foreground and Background Means

For each slide/experiment, the trimmed foreground mean and the trimmed background mean of all spots are first calculated, suppose nSpots, on each slide. For _each spot, if the data is acceptable (flag=0), we calculate the raw foreground mean and background mean are calculated by subtracting the background median from the foreground median for each spot. This is designated as a spot's “original value”. (Note: If flag=1, all values are set to 0.)

| original_value = fg_median - bg_median; | |
|---|---|
| if (flag == 0) | // experiment is successful |
| { | |
| raw_fg_mean = original_value; | |
| raw_bg_mean = bg_median; | |
| } | |
| else | // experiment is failed |

{
raw_fg_mean = 0.0;

raw_bg_mean = 0.0;

}

After that, the top and bottom 2% of data points are removed (trimmed) from the data set. After the above calculation, there are nSpot number of foreground means and background means for each slide/experiment, and both lists are sorted. For example:

| | raw_fg_mean[1], raw_fg_mean[2], ..., raw_fg_mean[N]; | N = 1, |
|---|---|---|
| nSpots; | | |
| | raw_bg_mean[1], raw_bg_mean[2], ..., raw_bg_mean[N]; | N = 1, |

nSpots;

the trimmed data points are calculated for each slide/experiment. Suppose a is the trimmed start data point and b is the trimmed end data point, there are:

a=ceil(nSpots*trim_percentage);

b=floor(nSpots*(1−trim_percentage);

The “background mean” is calculated from the background medians for the trimmed data set. For the background mean, the average background mean is simply calculated in interval [a, b] then assign to bg_mean:

bg_mean = (raw_bg_mean[a] + raw_bg_mean[a+1] +...+

raw_bg_mean[b] ) / (b−a+1) ;

The “foreground mean” is calculated from the “original values” (i.e. background-subtracted spot signal medians); only “original values” greater than 500 are used for this calculation (excluding the trimmed top and bottom 2% of the data). Suppose the sum of those foreground means is sum_raw_fg_mean and the amount of those foreground means is k.

fg_mean=sum_raw_fg_mean/k;

For clarity, a snippet code in Java looks like the following,

| | |
|---|---|
| int | k = 0; |

double sum_raw_fg_mean = 0.0;
for (int j = a; j < b; j++) {

if ( raw_fg_mean [j] > 500 ) {

sum_raw_fg_mean = sum_raw_fg_mean +
raw_fg_mean [j];
k++;

}

}
fg_mean = sum_raw_fg_mean / k;

After the calculation of trimmed foreground means and background means for all slides is complete, the normalization procedure is started.

### Step 2

### Normalize Data

For each slide a normalization coefficient is calculated which compares the foreground mean of the slide to a fixed maximum foreground mean (2200). This coefficient is:

coeff=max_fg_mean/fg_mean;

The normalized value of each spot is then calculated by multiplying the spot's “original value” by the normalization coefficient. Note that if this value is greater than the maximum reading of 65,000, then the value of 65,000 is used as the normalized value. Also note that if a spot's “original value” is less than the background value, the background value is used.

Recall that origrinal_value =
fg_median − bg_median
if ( original_value > bg_mean ) {
normalized_value = min(coeff * original_value,
MAX_VALUE);
} else {
normalized_value = coeff*bg_mean;
}

The normalized_value for each spot is the final (normalized) value used in the analysis

Threshhold for CuraChip Data Analysis

A number of control spots are present on CuraChip 1.2 for efficiency calculations and to provide alternative normalization methods. For example, CuraChip 1.2 contains a number of empty or negative control spots, as well as positive control spots containing a dilution series of oligos that detect the highly-expressed genes Ubiquitin and glyceraldehyde-3-phosphate dehydrogenase (GAPD). An analysis of spot signal level was performed using raw data from 67 hybridizations using all oligos. The maximum signal intensity for each oligo across all 67 hybridizations was determined, and the fold-over-background for this maximum signal was calculated (i.e. if the background reading is 20 and the raw spot intensity is 100, then the fold-over-background for that spot is 5×). The negative control or empty spots do occasionally “fire” or give a signal over the background level; however, they do not fire very strongly, with 77.1% of empty spots firing<3×over background and 91.7% <5× (see burgundy bars in figure below). The positive control spots (Ubiquitin and GAPD, the light blue and dark blue bars, respectively) always fired at >100× background. The experimental oligos (CuraOligos, in yellow below) fired over the entire range of intensities, with some at low fold-over-background intensities. Since the negative control spots do fire occasionally at low levels, we have set a suggested threshhold for data analysis at >5× background.

CG180777-01

Results of PTG Chip 1.2: One hundred seventy-eight samples of RNA from tissues obtained from surgically dissected tumors, non-diseased tissues from the corresponding organs and tumor xenografts grown in nude nu/nu mices were used to generate probes and run on PTG Chip 1.2. An oligo (optg2_{—}0014957) that corresponds to CG180777-01 on the PTG Chip 1.2 was scrutinized for its expression profile. The statistical analyses identify strong expression in lung, melanomas and breast cancers.

Thus, based upon its profile, the expression of this gene could be of use as a marker for subsets of lung, melanomas and breast cancers, in addition to the subset of Kidney cancers as previously disclosed. In addition, therapeutic inhibition of the activity of the product of this gene, through the use of antibodies or small molecule drugs, may be useful in the therapy of lung, melanomas and breast cancers that express CG180777-01 and are dependent on them.

Expression analysis of CG180777-01 using PTG Chip 1.2: Approximately 418 samples of RNA from tissues obtained from surgically dissected disease- and non-disease tissues, and treated and untreated cell lines, were used to generate labelled nucleic acid which was hybridized to PTG Chip 1.2. An oligo (optg2_{—}0014957) that corresponds to CG180777-01 on the PTG Chip 1.2 was analyzed for its expression profile (Table 1).

This gene shows low expression in number of cancer cell lines, normal and cancer samples from lung, prostate, pancreas, breast, pancreas and kidney. Expression of this gene is upregulated in pancreatic and breast cancer. Therefore, expression of this gene may be used as marker to detect the presence of breast and pancreatic cancer. Furthermore, therapeutic modulation of the expression or function of this gene may be effective in the treatment of lung, pancreatic, breast, prostate, and kidney cancers.

In addition, this gene is expressed at low levels in the brains, including amygdala, anterior cingulate, thalamus, hippocampus and astrocytes. Therefore, therapeutic modulation of this gene product may be useful in the treatment of central nervous system disorders such as Parkinson's disease, epilepsy, multiple sclerosis, schizophrenia and depression.

This gene is also expressed at significant level in thyroid, and pituitary glands. Therefore, therapeutic modulation of the activity of this gene may prove useful in the treatment of endocrine/metabolically related diseases, such as obesity and diabetes.

Significant expression of this gene is also seen in fetal lung. Therefore, therapeutic modulation of this gene may be useful in the treatment of lung related diseases especially development related diseases.

Significant expression of this gene is also seen in resting and activated monocytes (THP1) cells. Upon activation with pathogens such as LPS, monocytes contribute to the innate and specific immunity by migrating to the site of tissue injury and releasing inflammatory cytokines. This release contributes to the inflammation process. Therefore, modulation of the expression of the protein encoded by this transcript may prevent the recruitment of monocytes and the initiation of the inflammatory process, and reduce the symptoms of patients suffering from autoimmune and inflammatory diseases such as asthma, allergies, inflammatory bowel disease, lupus erythematosus, or rheumatoid arthritis.

TABLE E1

| | Foreground | Background | optg2_{—} |
|---|---|---|---|
| Sample | Mean | Mean | 1201117 |
| Lung cancer (35C) | 2536.51 | 22.17 | 17 |
| Lung NAT (36A) | 2733.37 | 20.31 | 152 |
| Lung cancer (35E) | 2933.33 | 21.31 | 140 |
| Lung cancer (365) | 3808.15 | 19.58 | 180 |
| Lung cancer (368) | 3824.5 | 21.07 | 115.5 |
| Lung cancer (369) | 2825.08 | 18.76 | 550 |
| Lung cancer (36E) | 4152.87 | 26.78 | 60 |
| Lung NAT (36F) | 3538.73 | 23.55 | 207.5 |
| Lung cancer (370) | 4143.89 | 21.18 | 69 |
| Lung cancer (376) | 2446.38 | 20.81 | 26 |
| Lung cancer (378) | 3989.95 | 27.35 | 30 |
| Lung cancer (37A) | 4136.72 | 36.64 | 66 |
| Normal Lung 4 | 4083.27 | 28.46 | 168 |
| Normal Lung 5 | 4235.38 | 25.22 | 138 |
| CuraChip reference 1 | 3728.44 | 28.62 | 214 |
| Melanoma | 2915.57 | 20.5 | 87.5 |
| Melanoma | 2646.56 | 20.29 | 98 |
| Melanoma (19585) | 2509.13 | 23.23 | 38 |
| Normal Lung 1 | 2759.91 | 24.22 | 163.5 |
| Lung cancer (372) | 3803.04 | 27.08 | 63 |
| Lung NAT (35D) | 3771.95 | 25.68 | 283 |
| Lung NAT (361) | 2214.53 | 20.77 | 25 |
| Melanoma | 2134.94 | 21.43 | 62 |
| Normal Lung 2 | 3656.2 | 20.99 | 381 |
| Lung cancer (374) | 3295.08 | 24.19 | 746 |
| Lung cancer (36B) | 3776.14 | 21.32 | 175 |
| Lung cancer (362) | 1543.94 | 26.44 | 34 |
| Lung cancer (358) | 1929.4 | 30.01 | 83 |
| Melanoma | 2375.7 | 20.83 | 81 |
| Normal Lung 3 | 3157.31 | 22.69 | 255.5 |
| Lung NAT (375) | 4614.72 | 32.86 | 514 |
| Lung cancer (36D) | 2785.76 | 24.74 | 31 |
| Lung NAT (363) | 4348.91 | 34.21 | 247 |
| Lung cancer (35A) | 3986.34 | 29.19 | 184 |
| Melanoma | 2189.36 | 20.44 | 27 |
| Prostate cancer (B8B) | 2957.66 | 9.6 | 277 |
| Prostate cancer (B88) | 4126.76 | 33.25 | 389 |
| Prostate NAT (B93) | 3378.81 | 37.92 | 286 |
| Prostate cancer (B8C) | 3527 | 42.55 | 273.5 |
| Prostate cancer (AD5) | 4105.44 | 45.35 | 395 |
| Prostate NAT (AD6) | 4196.5 | 41.71 | 522 |
| Prostate cancer (AD7) | 2830.59 | 42.73 | 193.5 |
| Prostate NAT (AD8) | 3404.14 | 29.72 | 346 |
| Prostate cancer (ADA) | 3700.09 | 34.54 | 715 |
| Prostate NAT (AD9) | 3022.26 | 30.92 | 255.5 |
| Prostate cancer (9E7) | 3084.26 | 30.48 | 261.5 |
| Prostate cancer (A0A) | 3983.11 | 24.56 | 499 |
| Prostate cancer (9E2) | 2889.43 | 23.94 | 426 |
| Pancreatic cancer (9E4) | 4473.72 | 23.53 | 400.5 |
| Pancreatic cancer (9D8) | 3443.44 | 20.25 | 193.5 |
| Pancreatic cancer (9D4) | 3819.27 | 17.3 | 315 |
| Pancreatic cancer (9BE) | 3287.48 | 24.17 | 588 |
| Pancreatic NAT (ADB) | 2358 | 28.92 | 63.5 |
| Pancreatic NAT (ADC) | 2863.88 | 36.96 | 42 |
| Pancreatic NAT (ADD) | 3118.81 | 30.22 | 32 |
| Pancreatic NAT (AED) | 3211.96 | 26.31 | 102 |
| Colon cancer (8A3) | 1984.83 | 48.06 | 51 |
| Colon NAT (8B6) | 1682.5 | 39.46 | 61 |
| Colon NAT (9F1) | 2378.93 | 48.8 | 74 |
| Colon cancer (9F2) | 1931.28 | 46.1 | 20 |
| Colon NAT (A1D) | 2029.41 | 46.05 | 65 |
| Colon cancer (9DB) | 2278.96 | 44.08 | 16 |
| Colon NAT (A15) | 1674.01 | 45.41 | 31 |
| Colon cancer (A14) | 1360.97 | 35.04 | 11 |
| Colon NAT (ACB) | 1707.6 | 45.03 | 29 |
| Colon cancer (AC0) | 1894.33 | 46.06 | 16 |
| Colon cancer (8A4) | 1785.56 | 43.34 | 10 |
| Colon NAT (ACD) | 1797.75 | 44.1 | 48 |
| Colon cancer (AC4) | 2198.75 | 49.26 | 28 |
| Colon NAT (AC2) | 1847.84 | 43.83 | 32.5 |
| Colon cancer (AC1) | 1806.35 | 39.49 | 15 |
| Colon NAT (ACC) | 2013.34 | 39.08 | 68 |
| Colon cancer (AC3) | 1539.46 | 47.71 | 16.5 |
| Breast cancer (9B7) | 1857.03 | 46.65 | 122 |
| Breast NAT (9CF) | 1462.79 | 47.35 | 7 |
| Breast cancer (9B6) | 2133.12 | 47.71 | 54 |
| Breast cancer (9C7) | 2302.99 | 47.48 | 116 |
| Colon cancer (8A6) | 2093.72 | 45.39 | 71 |
| Breast NAT (A11) | 1508.35 | 45.43 | 29 |
| Breast cancer (A1A) | 2246.51 | 46.55 | 29 |
| Breast cancer (9F3) | 1881.09 | 46.54 | 30 |
| Breast cancer (9B8) | 2174.46 | 48.66 | 69 |
| Breast NAT (9C4) | 1670.58 | 48.93 | 43 |
| Breast cancer (9EF) | 1168.07 | 23.61 | 29.5 |
| Breast cancer (9F0) | 1506.95 | 28.54 | 55 |
| Breast cancer (9B4) | 1016.05 | 32.36 | 9 |
| Breast cancer (9EC) | 2526.83 | 47.27 | 74 |
| Colon cancer (8A7) | 1594.35 | 48.21 | 5 |
| Colon cancer (8B7) | 2091.33 | 40.64 | 31 |
| Colon cancer (8A9) | 2533.34 | 40.66 | 102 |
| Colon cancer (8AB) | 1638.43 | 30.62 | 10 |
| Colon cancer (8AC) | 1975.26 | 41.39 | 13 |
| Colon NAT (8AD) | 1851.09 | 49.21 | 86 |
| Colon cancer (8B5) | 1920.15 | 47.11 | 22.5 |
| Cervical cancer (B08) | 1393.31 | 2.02 | 0 |
| Brain cancer (9F8) | 1400.44 | 8.86 | 25.5 |
| Brain cancer (9C0) | 655.35 | 4.73 | 0 |
| Brain cancer (9F7) | 1403.07 | 0.42 | 9.5 |
| Brain cancer (A00) | 1509.09 | 3.25 | 3.5 |
| Brain NAT (A01) | 1159.94 | 0.43 | 11 |
| Brain cancer (9DA) | 1019.67 | 0.72 | 0.5 |
| Brain cancer (9FE) | 1352.85 | 2.77 | 5 |
| Brain cancer (9C6) | 1237.61 | 3.47 | 0 |
| Brain cancer (9F6) | 917.48 | 2.17 | 0 |
| Cervical NAT (AEB) | 826.9 | 1.64 | 0 |
| Bladder NAT (23954) | 521.75 | 0.44 | 0 |
| Urinary cancer (AF6) | 1007.77 | 1.4 | 0 |
| Urinary cancer (B0C) | 1256.43 | 1.31 | 0 |
| Urinary cancer (AE4) | 1219.17 | 1.23 | 2.5 |
| Urinary NAT (B20) | 1222.48 | 1.23 | 0 |
| Urinary cancer (AE6) | 1114.91 | 1.03 | 0 |
| Urinary NAT (B04) | 655.35 | 0.07 | 0 |
| Urinary cancer (B07) | 543.73 | 1.64 | 0 |
| Urinary NAT (AF8) | 1247.4 | 0.53 | 0 |
| Cervical cancer (AFF) | 1411.18 | 4.31 | 0 |
| Ovarian cancer (9D7) | 1221.47 | 0.63 | 35 |
| Urinary cancer (AF7) | 1138.73 | 1.3 | 0 |
| Ovarian cancer (9F5) | 1298.98 | 0 | 5.5 |
| Ovarian cancer (A05) | 1134.77 | 2.16 | 17 |
| Ovarian cancer (9BC) | 505 | 0.15 | 0 |
| Ovarian cancer (9C2) | 1025.23 | 0.93 | 0 |
| Ovarian cancer (9D9) | 1203.34 | 1.53 | 0 |
| Ovarian NAT (AC7) | 685.35 | 0.54 | 0 |
| Ovarian NAT (AC9) | 716.79 | 0.85 | 0 |
| Ovarian NAT (ACA) | 628.62 | 2.38 | 0 |
| Cervical NAT (B1E) | 1293.21 | 7.01 | 0 |
| Ovarian NAT (AC5) | 542.12 | 0.99 | 0 |
| Cervical cancer (B00) | 1512.53 | 9.92 | 0 |
| Cervical NAT (AFA) | 1136.08 | 8.76 | 0 |
| Cervical cancer (B1F) | 1782.82 | 18.96 | 0 |
| Cervical NAT (B1C) | 655.35 | 2.36 | 0 |
| Brain cancer (9F9) | 1508.5 | 5.08 | 5.5 |
| Breast cancer (D34) | 2470.88 | 0 | 70 |
| Breast cancer (D35) | 2602.08 | 0 | 63 |
| Breast cancer (D36) | 2909.53 | 0 | 130.5 |
| Breast cancer (D37) | 2811.77 | 0.05 | 173 |
| Breast cancer (D38) | 2986.78 | 0.38 | 150 |
| Breast cancer (D39) | 3026.22 | 0.04 | 286 |
| Breast cancer (D3A) | 3072.62 | 0.08 | 235 |
| Breast cancer (D3B) | 2571.28 | 0.02 | 99.5 |
| Breast cancer (D3C) | 3213.98 | 0.6 | 271 |
| Breast cancer (D3D) | 3484.57 | 2.5 | 135 |
| Breast cancer (D3E) | 2958.51 | 0.17 | 458 |
| Breast cancer (D3F) | 2937.01 | 1.88 | 135 |
| Breast cancer (D40) | 2751.61 | 1.2 | 369 |
| Breast cancer (D42) | 2171.59 | 0.8 | 145 |
| Breast cancer (D43) | 2962.09 | 4.5 | 166.5 |
| Breast cancer (D44) | 2558.08 | 2.95 | 176 |
| Breast cancer (D45) | 2667.3 | 3.59 | 177.5 |
| Breast cancer (D46) | 3190.77 | 2.25 | 190.5 |
| SK-MES | 2804.32 | 0.56 | 47 |
| HLaC-79 | 3402.37 | 0 | 83 |
| H226 | 2562.59 | 0 | 0 |
| HCT-116 | 4221.68 | 0.09 | 74 |
| IGROV-1 | 3243.07 | 0 | 130.5 |
| MX-1 | 3253.75 | 0 | 211 |
| C33A | 3249.59 | 0 | 265 |
| Daudi | 2333.08 | 0.01 | 0 |
| MV522 | 2727.7 | 0.94 | 95 |
| RWP-2 | 2906.49 | 0 | 28 |
| BON | 2502.53 | 0.01 | 93 |
| MiaPaCa | 3604.78 | 0 | 155 |
| H82 | 2357.18 | 2.19 | 98 |
| H69 | 2759.55 | 0.12 | 0 |
| Caki-2 | 2687.93 | 0 | 72 |
| LNCaP | 3352.46 | 0.41 | 135.5 |
| A549 | 2593.12 | 0 | 0 |
| DU145 | 3970.51 | 0.07 | 78 |
| OVCAR-3 | 3230.65 | 0.14 | 275 |
| HT-29 | 3381.64 | 0.07 | 137 |
| DLD-2 | 3610.05 | 0.24 | 39.5 |
| MCF-7 | 3326.73 | 1.78 | 264 |
| H460 | 2464.22 | 0 | 0 |
| SW620 | 2732.11 | 0 | 0 |
| SK-OV-3 | 3519.75 | 0 | 101.5 |
| MDA-231 | 3464.04 | 0.04 | 0 |
| Caki-1 | 3801.64 | 0 | 88.5 |
| PC-3 | 2214.23 | 0 | 0 |
| LoVo | 3237.95 | 0 | 27 |
| Kidney NAT (10B1) | 3041.27 | 6.44 | 235.5 |
| Kidney cancer (10B2) | 3798.53 | 1.31 | 622.5 |
| Kidney NAT (10B3) | 3315.43 | 0 | 168 |
| Kidney cancer (10B4) | 3519.14 | 0.16 | 89.5 |
| Kidney NAT (10B5) | 3017.75 | 0 | 106.5 |
| Kidney cancer (10B6) | 3702.61 | 0 | 69.5 |
| Kidney NAT (10B7) | 3060.89 | 0 | 103.5 |
| Kidney cancer (10BA) | 3437.01 | 0 | 86.5 |
| Kidney NAT (10BB) | 3157.99 | 0 | 159.5 |
| Kidney cancer (10C0) | 3590.87 | 0.77 | 495 |
| Kidney NAT (10C1) | 3012.3 | 0 | 164.5 |
| Kidney cancer (10C4) | 3186.48 | 0.02 | 157 |
| Kidney NAT (10C5) | 3618.74 | 0 | 79 |
| Kidney cancer (10A8) | 3514.51 | 0 | 35.5 |
| Kidney NAT (10A9) | 2771.76 | 1.14 | 156.5 |
| Kidney cancer (10AA) | 3793.55 | 0.4 | 250.5 |
| Kidney NAT (10AB) | 2978.06 | 0 | 166 |
| Kidney cancer (10AC) | 3656.35 | 0.05 | 188 |
| Kidney NAT (10AD) | 3299.97 | 2.13 | 228 |
| Kidney cancer (10AE) | 3456.21 | 0.57 | 169.5 |
| Kidney NAT (10AF) | 2593.7 | 0 | 155 |
| Kidney cancer (10B0) | 3529.2 | 0.67 | 283.5 |
| Lymphoma (9BF) | 2333.81 | 0 | 0 |
| Lymphoma (9D2) | 1327.77 | 0 | 0 |
| Lymphoma (A04) | 1450.41 | 0 | 14 |
| Lymphoma (9DD) | 1095.68 | 0 | 0 |
| Lymphoma (F68) | 865.62 | 0 | 0 |
| Lymphoma (F6A) | 862.12 | 0 | 0 |
| Lymphoma (F6B) | 624.66 | 0 | 0 |
| Lymphoma (F6C) | 1621.52 | 0 | 0 |
| Lymphoma (F6D) | 864.76 | 0 | 0 |
| Lymphoma (F6E) | 1030.71 | 0 | 0 |
| Lymphoma (F6F) | 1120.14 | 0 | 0 |
| Lymphoma (F70) | 891.53 | 0 | 0 |
| Lymphoma (F71) | 915.92 | 0 | 0 |
| Lymphoma (F72) | 1199.28 | 3.13 | 0 |
| Lymphoma (F73) | 1357.46 | 0 | 0 |
| Lymphoma (F74) | 993.02 | 0 | 0 |
| Lymphoma NAT (1002) | 2069.54 | 0 | 0 |
| Lymphoma NAT (1004) | 1928.51 | 0 | 0 |
| Lymphoma NAT (1005) | 1412.32 | 0 | 0 |
| Lymphoma NAT (1007) | 1668.28 | 0 | 0 |
| Lymphoma NAT (1003) | 1919.49 | 0 | 0 |
| Lymphoma (9E3) | 1791.94 | 0 | 0 |
| Lymphoma (9D0) | 1530.59 | 0 | 0 |
| Lymphoma (9E1) | 1677.97 | 0 | 0 |
| Lymphoma (A0D) | 2656.34 | 1.4 | 1.5 |
| Lymphoma (9B5) | 2336.21 | 0 | 0 |
| Lymphoma (9D3) | 1902.13 | 0 | 0 |
| Normal Lung 4 | 4083.27 | 28.46 | 90.52 |
| Normal Lung 5 | 4235.38 | 25.22 | 71.68 |
| Normal Lung 1 | 2759.91 | 24.22 | 130.33 |
| Normal Lung 2 | 3656.2 | 20.99 | 229.25 |
| Normal Lung 3 | 3157.31 | 22.69 | 178.03 |
| SW1353 resting 1 h | 3946.45 | 0.6 | 22.86 |
| SW1353 resting 6 h | 3263.46 | 0.12 | 17.86 |
| SW1353 resting 16 h | 2311.8 | 0 | 0 |
| SW1353 IL-1b (1 ng/) 1 h | 2686.83 | 0.16 | 7.37 |
| SW1353 IL-1b (1 ng/) 6 h | 3159.94 | 2 | 6.27 |
| SW1353 IL-1b (1 ng/) 16 h | 3557.88 | 0.62 | 17.93 |
| SW1353 FGF20 (1 ug/) | 3512.56 | 0.69 | 26.93 |
| 1 h | | | |
| SW1353 FGF20 (1 ug/) | 2510.14 | 0.06 | 1.75 |
| 16 h | | | |
| SW1353 FGF20 (5 ug/) | 3448.11 | 1.01 | 22.33 |
| 1 h | | | |
| SW1353 FGF20 (5 ug/) | 3598.07 | 0.64 | 18.65 |
| 6 h | | | |
| SW1353 FGF20 (5 ug/) | 3687.34 | 4.42 | 21.78 |
| 16 h | | | |
| SW1353 FGF20 (1 ug/) | 3569.19 | 0.17 | 20.34 |
| IL-1b (1 ng/) 6 h | | | |
| SW1353 FGF20 (1 ug/) | 3970.28 | 0.01 | 36.57 |
| IL-1b (1 ng/) 16 h | | | |
| SW1353 FGF20 (5 ug/) | 3011.45 | 0 | 5.11 |
| IL-1b (1 ng/) 1 h | | | |
| SW1353 FGF20 (5 ug/) | 3184.88 | 2.69 | 31.43 |
| IL-1b (1 ng/) 6 h | | | |
| THP-1 aCD40 (1 ug/) 1 h | 3545.99 | 0.07 | 181.16 |
| THP-1 aCD40 (1 ug/) 6 h | 2882.56 | 0.44 | 76.7 |
| THP-1 LPS (100 ng/) 1 h | 3131.64 | 0.74 | 129.26 |
| THP-1 LPS (100 ng/) 6 h | 2356.5 | 0.05 | 19.61 |
| CCD1070SK TNFa (5 | 2888.01 | 0.78 | 32.76 |
| ng/) 6 h | | | |
| CCD1070SK TNFa (5 | 3029 | 0.39 | 51.57 |
| ng/) 24 h | | | |
| CCD1070SK IL-1b (1 ng/) | 3307.91 | 5.76 | 35.58 |
| 24 h | | | |
| THP-1 resting | 3080.68 | 0.87 | 157.47 |
| THP-1 aCD40 (1 ug/) 24 h | 2032.49 | 0.82 | 33.55 |
| THP-1 LPS (100 ng/) 24 h | 1597.29 | 3.58 | 44.76 |
| CCD1070SK IL-1b (1 ng/) | 3026.56 | 3.51 | 43.61 |
| 6 h | | | |
| LC 18 hr | 2725.87 | 0.36 | 34.7 |
| LC-IL-! 18 hr | 1474.79 | 2.66 | 0 |
| Astrocyte_IL1B_1 hr_a | 3116.77 | 10.04 | 264.34 |
| Astrocyte_IL1B_6 hr_a | 3119.7 | 16.51 | 357.18 |
| Astrocyte_IL1B_24 hr_a | 3142.98 | 13.81 | 335.99 |
| SHSY 5Y Undifferentiated | 3166.23 | 14.61 | 105.61 |
| SHSY 5Y Differentiated | 2959.01 | 8.5 | 100.74 |
| LC 0 hr | 1880.7 | 0 | 0 |
| Normal Fetal Kidney | 2011.8 | 0 | 7.65 |
| Normal Liver | 2053.67 | 3.81 | 0 |
| Normal Fetal Liver | 3555.17 | 0 | 3.09 |
| Normal Fetal Lung | 4164.55 | 0 | 208.4 |
| Normal Salivary Gland | 3466.36 | 0 | 0 |
| Normal Fetal Skeletal | 2504.59 | 0 | 0 |
| Muscle | | | |
| Normal Thyroid | 3566.17 | 0 | 193.71 |
| Normal Trachea | 3596.15 | 0 | 61.18 |
| LC-IL-1 0 hr | 2560.23 | 0 | 0.86 |
| Heart pool | 3167.78 | 0 | 0 |
| Pituitary Pool | 2908.48 | 0 | 316.94 |
| Spleen Pool | 2068.62 | 0 | 5.32 |
| Stomach Pool | 2826.7 | 0 | 9.34 |
| Testis Pool | 3348.7 | 0 | 268.04 |
| Thymus Pool | 2653.82 | 0 | 45.59 |
| Small Intestine - 5 donor | 3795.64 | 0 | 68.39 |
| pool | | | |
| Lymph node - 5 donor | 4339.52 | 0 | 69.45 |
| pool | | | |
| Kidney - 5 donor pool | 3347.34 | 0 | 138.02 |
| Jurkat Resting | 3779.74 | 0.48 | 139.11 |
| Jurkat CD3 (500 ng/ml) | 2459.46 | 1.68 | 71.56 |
| 6 hr A | | | |
| Jurkat CD3 (500 ng/ml) | 1897.6 | 0.08 | 71.3 |
| 24 hr A | | | |
| Jurkat CD3 | 1867.35 | 0.68 | 24.74 |
| (500 ng/ml) + CD28 (1 ug/ml) | | | |
| 6 hr A | | | |
| Jurkat CD3 | 1574.07 | 0.35 | 60.1 |
| (500 ng/ml) + CD28 (1 ug/ml) | | | |
| 24 hr A | | | |
| control (no treatment)_1 | 5400.28 | 2.01 | 65.59 |
| hr | | | |
| 10 ng/ml IL-1b_1 hr | 5250.91 | 1.51 | 70.39 |
| 10 ng/ml TNF-a_1 hr | 5668.06 | 2.55 | 80.73 |
| 200 uM BzATP_1 hr | 5619.87 | 0.31 | 89.65 |
| control (no treatment)_5 | 5630.13 | 1.02 | 77.56 |
| hr | | | |
| 10 ng/ml IL-1b_5 hr | 6332.12 | 10.52 | 70.36 |
| 10 ng/ml TNF-a_5 hr | 6070.17 | 6.37 | 75.93 |
| 200 uM BzATP_5 hr | 6425.22 | 2.39 | 99.64 |
| control (no treatment)_24 | 4825.87 | 3.5 | 62 |
| hr | | | |
| 10 ng/ml IL-1b_24 hr | 5349.28 | 9.73 | 73.41 |
| 10 ng/ml TNF-a_24 hr | 5672.31 | 4.97 | 62.25 |
| 200 uM BzATP_24 hr | 4814.1 | 0.51 | 70.61 |
| Alzheimer's disease | 1854.86 | 14.76 | 33.21 |
| B4951 | | | |
| Alzheimer's disease | 2540.02 | 19.95 | 108.27 |
| B4953 | | | |
| Alzheimer's disease | 1757.68 | 22.42 | 45.06 |
| B5018 | | | |
| Alzheimer's disease | 1491.9 | 18.37 | 7.37 |
| B5019 | | | |
| Alzheimer's disease | 2247.49 | 18.04 | 66.56 |
| B5086 | | | |
| Alzheimer's disease | 2150.92 | 19.76 | 38.87 |
| B5096 | | | |
| Alzheimer's disease | 732.56 | 15.93 | 21.02 |
| B5098 | | | |
| Alzheimer's disease | 1841.99 | 18.04 | 9.55 |
| B5129 | | | |
| Alzheimer's disease | 3233.87 | 21.56 | 168.37 |
| B5210 | | | |
| Control B4810 | 2987.22 | 21.85 | 155.76 |
| Control B4825 | 2903.91 | 18.74 | 187.88 |
| Control B4930 | 2287.12 | 22.69 | 30.78 |
| Control B4932 | 3424.98 | 20.12 | 370.95 |
| Control B5024 | 3859.32 | 22.42 | 398.46 |
| Control B5113 | 1897 | 17.87 | 112.49 |
| Control B5140 | 1901.93 | 18.88 | 68.25 |
| Control B5190 | 1284.69 | 15.26 | 53.94 |
| Control B5220 | 2225.75 | 18.7 | 192.25 |
| Control B5245 | 2119.39 | 21.5 | 143.25 |
| AH3 B3791 | 2202.74 | 19.73 | 173.78 |
| AH3 B3855 | 1849.89 | 17.93 | 267.58 |
| AH3 B3877 | 2144.15 | 17.01 | 402.72 |
| AH3 B3893 | 2103.76 | 16.34 | 81.57 |
| AH3 B3894 | 1820.82 | 17.31 | 166.74 |
| AH3 B3949 | 1607.86 | 23.9 | 58.84 |
| AH3 B4477 | 1602.58 | 20.32 | 123.55 |
| AH3 B4540 | 2260.92 | 22.79 | 252.02 |
| AH3 B4577 | 2142.42 | 22.59 | 257.75 |
| AH3 B4639 | 1550.43 | 21.89 | 28.38 |
| Schizophrenia | 2468.43 | 21.23 | 58.82 |
| hippocampus 683 | | | |
| Depression hippocampus | 1473.83 | 18 | 0 |
| 487 | | | |
| Depression hippocampus | 2481.12 | 10.69 | 58.97 |
| 600 | | | |
| Normal hippocampus | 2624.25 | 33.19 | 18.44 |
| 2407a | | | |
| Normal hippocampus | 2114.72 | 21.4 | 19.25 |
| 1042 | | | |
| Depression hippocampus | 1448.13 | 10.6 | 21.27 |
| 2767 | | | |
| Depression hippocampus | 1836.77 | 47.98 | 19.16 |
| 567 | | | |
| Control hippocampus | 2752.14 | 14.38 | 96.72 |
| 3175 | | | |
| Depression hippocampus | 1735.6 | 9.4 | 30.42 |
| 3096 | | | |
| Depression hippocampus | 2784.26 | 17.28 | 41.09 |
| 1491 | | | |
| Depression hippocampus | 2241.25 | 16.73 | 28.47 |
| 2540 | | | |
| Schizophrenia | 1923.26 | 16.97 | 56.05 |
| hippocampus 2798 | | | |
| Control hippocampus | 2605.59 | 14.97 | 33.77 |
| 1973 | | | |
| Normal hippocampus and | 2031.45 | 13.85 | 34.11 |
| amygdala 2601 | | | |
| Schizophrenia | 1621.43 | 21.03 | 0 |
| hippocampus 2785 | | | |
| Schizophrenia | 3271.31 | 39.54 | 62.21 |
| hippocampus 484 | | | |
| Normal hippocampus | 2806.68 | 21.69 | 50.95 |
| 2556 | | | |
| Depression hippocampus | 2705.56 | 15.83 | 32.53 |
| 1158 | | | |
| Control hippocampus 552 | 3378.83 | 21.65 | 161.48 |
| Schizophrenia | 2304.06 | 12.06 | 0 |
| hippocampus 1737 | | | |
| Normal hippocampus | 3335.3 | 17.54 | 67.28 |
| 1239 | | | |
| Normal hippocampus | 3068.38 | 14.64 | 49.47 |
| 1465 | | | |
| Normal hippocampus | 1323.77 | 4.6 | 0 |
| 3080 | | | |
| Normal hippocampus 738 | 4229.67 | 12.96 | 146.16 |
| Schizophrenia | 2067.82 | 12.78 | 6.92 |
| hippocampus 2586 | | | |
| Normal hippocampus | 3306.02 | 13.41 | 156.38 |
| 2551 | | | |
| Depression hippocampus | 2352.1 | 13.53 | 75.76 |
| 588 | | | |
| Depression hippocampus | 3291.7 | 13.05 | 127.65 |
| 529 | | | |
| Depression hippocampus | 1686.08 | 11.37 | 27.4 |
| and dentate gyrus | | | |
| Schizophrenia amygdala | 2136.57 | 0.55 | 40.16 |
| 2586 | | | |
| Normal substantia nigra | 1240.83 | 0.03 | 0 |
| 234 | | | |
| Normal substantia nigra | 1515.69 | 14.19 | 0 |
| 1065 | | | |
| formal substantia nigra | 1341.77 | 0 | 0 |
| 3236 | | | |
| Normal substantia nigra | 3718.91 | 0.43 | 64.19 |
| 2551 | | | |
| Normal substantia nigra | 1003.1 | 0 | 0 |
| 1597 | | | |
| Control thalamus 552 | 948.15 | 0.02 | 4.64 |
| Control thalamus 566 | 1698.42 | 0 | 0 |
| Control thalamus 606 | 2625.59 | 0.31 | 96.78 |
| Control thalamus 738 | 2464.52 | 0 | 42.85 |
| Control thalamus 1065 | 2991.38 | 0 | 54.42 |
| Control thalamus 1092 | 2416.75 | 0 | 36.41 |
| Control thalamus 1597 | 2389.84 | 0 | 48.79 |
| Control thalamus 2253 | 1602.4 | 0 | 0 |
| Control thalamus 2551 | 3017.98 | 0 | 120.28 |
| Depression thalamus 588 | 2245.45 | 0.29 | 31.35 |
| Depression thalamus 600 | 1442.56 | 0 | 0 |
| Depression thalamus 721 | 1921.28 | 0 | 0 |
| Depression thalamus 728 | 3113.17 | 4.33 | 48.05 |
| Depression thalamus 759 | 2433.85 | 0 | 56.95 |
| Depression thalamus 881 | 2456.7 | 0 | 15.67 |
| Schizophrenia thalamus | 1952.08 | 0 | 3.38 |
| 477 | | | |
| Schizophrenia thalamus | 3553.95 | 0 | 68.71 |
| 532 | | | |
| Schizophrenia thalamus | 3798.02 | 0 | 50.11 |
| 683 | | | |
| Schizophrenia thalamus | 3260.82 | 0.03 | 44.87 |
| 544 | | | |
| Schizophrenia thalamus | 2246.95 | 0 | 0 |
| 1671 | | | |
| Schizophrenia thalamus | 1958.75 | 0.01 | 0 |
| 1737 | | | |
| Schizophrenia thalamus | 1953.64 | 0 | 56.31 |
| 2464 | | | |
| Schizophrenia thalamus | 3338.49 | 0 | 28.67 |
| 2586 | | | |
| Depression amygdala | 1936.43 | 0 | 9.09 |
| 600 | | | |
| Depression amygdala | 2378.2 | 0 | 69.38 |
| 759 | | | |
| Depression anterior | 2808.48 | 0 | 75.98 |
| cingulate 759 | | | |
| Control amygdala 552 | 3675.87 | 0.36 | 151.42 |
| Control anterior cingulate | 3115.46 | 0.67 | 236.56 |
| 482 | | | |
| Depression anterior | 1964.87 | 0 | 1.12 |
| cingulate 721 | | | |
| Control amygdala 3175 | 2674.16 | 0.04 | 134.92 |
| Depression anterior | 2389.83 | 0.06 | 37.74 |
| cingulate 600 | | | |
| Depression anterior | 2629.92 | 0 | 92.85 |
| cingulate 588 | | | |
| Control anterior cingulate | 3605.03 | 0.79 | 238.61 |
| 3175 | | | |
| Control anterior cingulate | 2414.64 | 0 | 273.33 |
| 606 | | | |
| Depression anterior | 2397.28 | 1.2 | 40.38 |
| cingulate 567 | | | |
| Depression amygdala | 3410.76 | 3.1 | 79.98 |
| 588 | | | |
| Control anterior cingulate | 2445.09 | 3.01 | 54.89 |
| 3080 | | | |
| Control anterior cingulate | 2520 | 5.45 | 75.08 |
| 2601 | | | |
| Control anterior cingulate | 3118.04 | 0.76 | 87.49 |
| 1042 | | | |
| Control anterior cingulate | 2913.66 | 1.45 | NA |
| 3236 | | | |
| Control amygdala 1502 | 4253.4 | 9.62 | 147.67 |
| Control anterior cingulate | 2624.08 | 8.86 | 0 |
| 807 | | | |
| Control amygdala 1597 | 3710.89 | 11.88 | 141.1 |
| Parkinson's substantia | 2457.51 | 0.25 | 11.64 |
| nigra 2842 | | | |
| Parkinson's substantia | 1548.58 | 0 | 0 |
| nigra 2917 | | | |
| Schizophrenia amygdala | 2009.89 | 0 | 71.15 |
| 544 | | | |
| Schizophrenia amygdala | 730.94 | 0 | 0 |
| 532 | | | |
| Depression amygdala | 2408.1 | 0 | 28.32 |
| 2540 | | | |
| Parkinson's substantia | 1544.99 | 0 | 0 |
| nigra 2899 | | | |
| Depression anterior | 3015.65 | 0 | 103.59 |
| cingulate 881 | | | |

CG181825-01

Results of PTG Chip 1.2: One hundred seventy-eight samples of RNA from tissues obtained from surgically dissected tumors, non-diseased tissues from the corresponding organs and tumor xenografts grown in nude nu/nu mices were used to generate probes and run on PTG Chip 1.2. An oligo (optg2_{—}1206388) that corresponds to CG181825-01 on the PTG Chip 1.2 was scrutinized for its expression profile. The statistical analysis identify strong expression in lung, melanomas and breast cancers.

Thus, based upon its profile, the expression of this gene could be of use as a marker for subsets of lung, melanomas and breast cancers, in addition to the subset of Kidney cancers as previously disclosed. In addition, therapeutic inhibition of the activity of the product of this gene, through the use of antibodies or small molecule drugs, may be useful in the therapy of lung, melanomas and breast cancers that express CG181825-01 and are dependent on them.

Expression analysis of CG181825-01 using PTG Chip 1.2: Approximately 418 samples of RNA from tissues obtained from surgically dissected disease- and non-disease tissues, and treated and untreated cell lines, were used to generate labelled nucleic acid which was hybridized to PTG Chip 1.2. An oligo (optg2_{—}1206388) that corresponds to CG181825-01 on the PTG Chip 1.2 was analyzed for its expression profile (Table 1).

This gene shows low expression in number of cancer cell lines, normal and cancer samples from lung, pancreas, breast, cervix and kidney.

In addition, this gene is expressed at low levels in the brains of an independent group of individuals, especially in substantia nigra and amygdala of samples from normal patients or patients suffering with Parkinson's diseases and schizophrenia.

This gene is also expressed at significant level in thyroid, pituitary glands, fetal liver, and stomach. Therefore, therapeutic modulation of the activity of this gene may prove useful in the treatment of endocrine/metabolically related diseases, such as obesity and diabetes.

TABLE E2

| | Foreground | Background | optg2_{—} |
|---|---|---|---|
| Sample | Mean | Mean | 1206388 |
| Lung cancer(35C) | 2536.51 | 22.17 | 33 |
| Lung NAT(36A) | 2733.37 | 20.31 | 47.5 |
| Lung cancer(35E) | 2933.33 | 21.31 | 60 |
| Lung cancer(365) | 3808.15 | 19.58 | 159 |
| Lung cancer(368) | 3824.5 | 21.07 | 114.5 |
| Lung cancer(369) | 2825.08 | 18.76 | 40 |
| Lung cancer(36E) | 4152.87 | 26.78 | 132 |
| Lung NAT(36F) | 3538.73 | 23.55 | 87 |
| Lung cancer(370) | 4143.89 | 21.18 | 40 |
| Lung cancer(376) | 2446.38 | 20.81 | 34 |
| Lung cancer(378) | 3989.95 | 27.35 | 30 |
| Lung cancer(37A) | 4136.72 | 36.64 | 123.5 |
| Normal Lung 4 | 4083.27 | 28.46 | 85 |
| Normal Lung 5 | 4235.38 | 25.22 | 95 |
| CuraChip reference 1 | 3728.44 | 28.62 | 49 |
| Melanoma | 2915.57 | 20.5 | 25 |
| Melanoma | 2646.56 | 20.29 | 18 |
| Melanoma (19585) | 2509.13 | 23.23 | 16.5 |
| Normal Lung 1 | 2759.91 | 24.22 | 32 |
| Lung cancer(372) | 3803.04 | 27.08 | 30 |
| Lung NAT(35D) | 3771.95 | 25.68 | 147 |
| Lung NAT(361) | 2214.53 | 20.77 | 49.5 |
| Melanoma | 2134.94 | 21.43 | 17 |
| Normal Lung 2 | 3656.2 | 20.99 | 69 |
| Lung cancer(374) | 3295.08 | 24.19 | 72 |
| Lung cancer(36B) | 3776.14 | 21.32 | 84 |
| Lung cancer(362) | 1543.94 | 26.44 | 40 |
| Lung cancer(358) | 1929.4 | 30.01 | 51 |
| Melanoma | 2375.7 | 20.83 | 22 |
| Normal Lung 3 | 3157.31 | 22.69 | 34 |
| Lung NAT(375) | 4614.72 | 32.86 | 96.5 |
| Lung cancer(36D) | 2785.76 | 24.74 | 66.5 |
| Lung NAT(363) | 4348.91 | 34.21 | 76 |
| Lung cancer(35A) | 3986.34 | 29.19 | 117 |
| Melanoma | 2189.36 | 20.44 | 16 |
| Prostate cancer(B8B) | 2957.66 | 9.6 | 70.5 |
| Prostate cancer(B88) | 4126.76 | 33.25 | 18.5 |
| Prostate NAT(B93) | 3378.81 | 37.92 | 16.5 |
| Prostate cancer(B8C) | 3527 | 42.55 | 10 |
| Prostate cancer(AD5) | 4105.44 | 45.35 | 0 |
| Prostate NAT(AD6) | 4196.5 | 41.71 | 23.5 |
| Prostate cancer(AD7) | 2830.59 | 42.73 | 0 |
| Prostate NAT(AD8) | 3404.14 | 29.72 | 25 |
| Prostate cancer(ADA) | 3700.09 | 34.54 | 1 |
| Prostate NAT(AD9) | 3022.26 | 30.92 | 10 |
| Prostate cancer(9E7) | 3084.26 | 30.48 | 0.5 |
| Prostate cancer(A0A) | 3983.11 | 24.56 | 30 |
| Prostate cancer(9E2) | 2889.43 | 23.94 | 23 |
| Pancreatic cancer(9E4) | 4473.72 | 23.53 | 165 |
| Pancreatic cancer(9D8) | 3443.44 | 20.25 | 24 |
| Pancreatic cancer(9D4) | 3819.27 | 17.3 | 8.5 |
| Pancreatic cancer(9BE) | 3287.48 | 24.17 | 43 |
| Pancreatic NAT(ADB) | 2358 | 28.92 | 0 |
| Pancreatic NAT(ADC) | 2863.88 | 36.96 | 23.5 |
| Pancreatic NAT(ADD) | 3118.81 | 30.22 | 1.5 |
| Pancreatic NAT(AED) | 3211.96 | 26.31 | 42 |
| Colon cancer(8A3) | 1984.83 | 48.06 | 13 |
| Colon NAT(8B6) | 1682.5 | 39.46 | 9 |
| Colon NAT(9F1) | 2378.93 | 48.8 | 2 |
| Colon cancer(9F2) | 1931.28 | 46.1 | 10.5 |
| Colon NAT(A1D) | 2029.41 | 46.05 | 4 |
| Colon cancer(9DB) | 2278.96 | 44.08 | 3.5 |
| Colon NAT(A15) | 1674.01 | 45.41 | 0 |
| Colon cancer(A14) | 1360.97 | 35.04 | 4 |
| Colon NAT(ACB) | 1707.6 | 45.03 | 1 |
| Colon cancer(AC0) | 1894.33 | 46.06 | 0 |
| Colon cancer(8A4) | 1785.56 | 43.34 | 5 |
| Colon NAT(ACD) | 1797.75 | 44.1 | 8 |
| Colon cancer(AC4) | 2198.75 | 49.26 | 4 |
| Colon NAT(AC2) | 1847.84 | 43.83 | 1 |
| Colon cancer(AC1) | 1806.35 | 39.49 | 9 |
| Colon NAT(ACC) | 2013.34 | 39.08 | 0 |
| Colon cancer(AC3) | 1539.46 | 47.71 | 2 |
| Breast cancer(9B7) | 1857.03 | 46.65 | 15 |
| Breast NAT(9CF) | 1462.79 | 47.35 | 0 |
| Breast cancer(9B6) | 2133.12 | 47.71 | 9 |
| Breast cancer(9C7) | 2302.99 | 47.48 | 18 |
| Colon cancer(8A6) | 2093.72 | 45.39 | 6 |
| Breast NAT(A11) | 1508.35 | 45.43 | 0 |
| Breast cancer(A1A) | 2246.51 | 46.55 | 1 |
| Breast cancer(9F3) | 1881.09 | 46.54 | 6 |
| Breast cancer(9B8) | 2174.46 | 48.66 | 2 |
| Breast NAT(9C4) | 1670.58 | 48.93 | 4 |
| Breast cancer(9EF) | 1168.07 | 23.61 | 4 |
| Breast cancer(9F0) | 1506.95 | 28.54 | 0 |
| Breast cancer(9B4) | 1016.05 | 32.36 | 0 |
| Breast cancer(9EC) | 2526.83 | 47.27 | 8 |
| Colon cancer(8A7) | 1594.35 | 48.21 | 0 |
| Colon cancer(8B7) | 2091.33 | 40.64 | 4 |
| Colon cancer(8A9) | 2533.34 | 40.66 | 24 |
| Colon cancer(8AB) | 1638.43 | 30.62 | 4 |
| Colon cancer(8AC) | 1975.26 | 41.39 | 9 |
| Colon NAT(8AD) | 1851.09 | 49.21 | 1 |
| Colon cancer(8B5) | 1920.15 | 47.11 | 5.5 |
| Cervical cancer(B08) | 1393.31 | 2.02 | 186 |
| Brain cancer(9F8) | 1400.44 | 8.86 | NA |
| Brain cancer(9C0) | 655.35 | 4.73 | 0 |
| Brain cancer(9F7) | 1403.07 | 0.42 | 0 |
| Brain cancer(A00) | 1509.09 | 3.25 | 0 |
| Brain NAT(A01) | 1159.94 | 0.43 | 6 |
| Brain cancer(9DA) | 1019.67 | 0.72 | 0 |
| Brain cancer(9FE) | 1352.85 | 2.77 | 0 |
| Brain cancer(9C6) | 1237.61 | 3.47 | 0 |
| Brain cancer(9F6) | 917.48 | 2.17 | 0 |
| Cervical NAT(AEB) | 826.9 | 1.64 | NA |
| Bladder NAT(23954) | 521.75 | 0.44 | 0 |
| Urinary cancer(AF6) | 1007.77 | 1.4 | 0 |
| Urinary cancer(B0C) | 1256.43 | 1.31 | 0 |
| Urinary cancer(AE4) | 1219.17 | 1.23 | 0 |
| Urinary NAT(B20) | 1222.48 | 1.23 | 0 |
| Urinary cancer(AE6) | 1114.91 | 1.03 | 0 |
| Urinary NAT(B04) | 655.35 | 0.07 | 0 |
| Urinary cancer(B07) | 543.73 | 1.64 | 1 |
| Urinary NAT(AF8) | 1247.4 | 0.53 | 0 |
| Cervical cancer(AFF) | 1411.18 | 4.31 | NA |
| Ovarian cancer(9D7) | 1221.47 | 0.63 | 13 |
| Urinary cancer(AF7) | 1138.73 | 1.3 | 0 |
| Ovarian cancer(9F5) | 1298.98 | 0 | NA |
| Ovarian cancer(A05) | 1134.77 | 2.16 | 3 |
| Ovarian cancer(9BC) | 505 | 0.15 | NA |
| Ovarian cancer(9C2) | 1025.23 | 0.93 | 0 |
| Ovarian cancer(9D9) | 1203.34 | 1.53 | NA |
| Ovarian NAT(AC7) | 685.35 | 0.54 | NA |
| Ovarian NAT(AC9) | 716.79 | 0.85 | NA |
| Ovarian NAT(ACA) | 628.62 | 2.38 | NA |
| Cervical NAT(B1E) | 1293.21 | 7.01 | NA |
| Ovarian NAT(AC5) | 542.12 | 0.99 | 0 |
| Cervical cancer(B00) | 1512.53 | 9.92 | 185 |
| Cervical NAT(AFA) | 1136.08 | 8.76 | NA |
| Cervical cancer(B1F) | 1782.82 | 18.96 | NA |
| Cervical NAT(B1C) | 655.35 | 2.36 | NA |
| Brain cancer(9F9) | 1508.5 | 5.08 | NA |
| Breast cancer(D34) | 2470.88 | 0 | 48.5 |
| Breast cancer(D35) | 2602.08 | 0 | 127 |
| Breast cancer(D36) | 2909.53 | 0 | NA |
| Breast cancer(D37) | 2811.77 | 0.05 | 75 |
| Breast cancer(D38) | 2986.78 | 0.38 | 96 |
| Breast cancer(D39) | 3026.22 | 0.04 | 78 |
| Breast cancer(D3A) | 3072.62 | 0.08 | 115.5 |
| Breast cancer(D3B) | 2571.28 | 0.02 | 31 |
| Breast cancer(D3C) | 3213.98 | 0.6 | 99.5 |
| Breast cancer(D3D) | 3484.57 | 2.5 | 73 |
| Breast cancer(D3E) | 2958.51 | 0.17 | 106 |
| Breast cancer(D3F) | 2937.01 | 1.88 | 30 |
| Breast cancer(D40) | 2751.61 | 1.2 | 31.5 |
| Breast cancer(D42) | 2171.59 | 0.8 | 19 |
| Breast cancer(D43) | 2962.09 | 4.5 | 44 |
| Breast cancer(D44) | 2558.08 | 2.95 | 24 |
| Breast cancer(D45) | 2667.3 | 3.59 | 39 |
| Breast cancer(D46) | 3190.77 | 2.25 | 53.5 |
| SK-MES | 2804.32 | 0.56 | 130.5 |
| HLaC-79 | 3402.37 | 0 | 103.5 |
| H226 | 2562.59 | 0 | 8 |
| HCT-116 | 4221.68 | 0.09 | 10 |
| IGROV-1 | 3243.07 | 0 | 32 |
| MX-1 | 3253.75 | 0 | 0 |
| C33A | 3249.59 | 0 | 11 |
| Daudi | 2333.08 | 0.01 | 65 |
| MV522 | 2727.71 | 0.94 | 0 |
| RWP-2 | 2906.49 | 0 | 0 |
| BON | 2502.53 | 0.01 | 9 |
| MiaPaCa | 3604.78 | 0 | 39 |
| H82 | 2357.18 | 2.19 | 15 |
| H69 | 2759.55 | 0.12 | 46.5 |
| Caki-2 | 2687.93 | 0 | 1 |
| LNCaP | 3352.46 | 0.41 | 64.5 |
| A549 | 2593.12 | 0 | 9 |
| DU145 | 3970.51 | 0.07 | 44 |
| OVCAR-3 | 3230.65 | 0.14 | 51 |
| HT-29 | 3381.64 | 0.07 | 41.5 |
| DLD-2 | 3610.05 | 0.24 | 4 |
| MCF-7 | 3326.73 | 1.78 | 48 |
| H460 | 2464.22 | 0 | 102 |
| SW620 | 2732.11 | 0 | 12 |
| SK-OV-3 | 3519.75 | 0 | NA |
| MDA-231 | 3464.04 | 0.04 | 72 |
| Caki-1 | 3801.64 | 0 | 87 |
| PC-3 | 2214.23 | 0 | 77.5 |
| LoVo | 3237.95 | 0 | 49 |
| Kidney NAT(10B1) | 3041.27 | 6.44 | 187 |
| Kidney cancer(10B2) | 3798.53 | 1.31 | 34 |
| Kidney NAT(10B3) | 3315.43 | 0 | 1197 |
| Kidney cancer(10B4) | 3519.14 | 0.16 | 73 |
| Kidney NAT(10B5) | 3017.75 | 0 | 177 |
| Kidney cancer(10B6) | 3702.61 | 0 | 13403.5 |
| Kidney NAT(10B7) | 3060.89 | 0 | 330 |
| Kidney cancer(10BA) | 3437.01 | 0 | 12600.5 |
| Kidney NAT(10BB) | 3157.99 | 0 | 308 |
| Kidney cancer(10C0) | 3590.87 | 0.77 | 40 |
| Kidney NAT(10C1) | 3012.3 | 0 | 157 |
| Kidney cancer(10C4) | 3186.48 | 0.02 | 270.5 |
| Kidney NAT(10C5) | 3618.74 | 0 | 64 |
| Kidney cancer(10A8) | 3514.51 | 0 | 14 |
| Kidney NAT(10A9) | 2771.76 | 1.14 | 241.5 |
| Kidney cancer(10AA) | 3793.55 | 0.4 | 86 |
| Kidney NAT(10AB) | 2978.06 | 0 | 222.5 |
| Kidney cancer(10AC) | 3656.35 | 0.05 | 26 |
| Kidney NAT(10AD) | 3299.97 | 2.13 | 436 |
| Kidney cancer(10AE) | 3456.21 | 0.57 | 49.5 |
| Kidney NAT(10AF) | 2593.7 | 0 | 100 |
| Kidney cancer(10B0) | 3529.2 | 0.67 | 35 |
| Lymphoma(9BF) | 2333.81 | 0 | 0 |
| Lymphoma(9D2) | 1327.77 | 0 | 0 |
| Lymphoma(A04) | 1450.41 | 0 | 0 |
| Lymphoma(9DD) | 1095.68 | 0 | 0 |
| Lymphoma(F68) | 865.62 | 0 | 0 |
| Lymphoma(F6A) | 862.12 | 0 | 0 |
| Lymphoma(F6B) | 624.66 | 0 | 0 |
| Lymphoma(F6C) | 1621.52 | 0 | 0 |
| Lymphoma(F6D) | 864.76 | 0 | 0 |
| Lymphoma(F6E) | 1030.71 | 0 | 0 |
| Lymphoma(F6F) | 1120.14 | 0 | 54 |
| Lymphoma(F70) | 891.53 | 0 | 0 |
| Lymphoma(F71) | 915.92 | 0 | 0 |
| Lymphoma(F72) | 1199.28 | 3.13 | 15 |
| Lymphoma(F73) | 1357.46 | 0 | 2 |
| Lymphoma(F74) | 993.02 | 0 | 0 |
| Lymphoma NAT(1002) | 2069.54 | 0 | 0 |
| Lymphoma NAT(1004) | 1928.51 | 0 | 0 |
| Lymphoma NAT(1005) | 1412.32 | 0 | 0 |
| Lymphoma NAT(1007) | 1668.28 | 0 | 0 |
| Lymphoma NAT(1003) | 1919.49 | 0 | 0 |
| Lymphoma(9E3) | 1791.94 | 0 | 0 |
| Lymphoma(9D0) | 1530.59 | 0 | 0 |
| Lymphoma(9E1) | 1677.97 | 0 | 0 |
| Lymphoma(A0D) | 2656.34 | 1.4 | 61 |
| Lymphoma(9B5) | 2336.21 | 0 | 8 |
| Lymphoma(9D3) | 1902.13 | 0 | 0 |
| Normal Lung 4 | 4083.27 | 28.46 | 45.8 |
| Normal Lung 5 | 4235.38 | 25.22 | 49.35 |
| Normal Lung 1 | 2759.91 | 24.22 | 25.51 |
| Normal Lung 2 | 3656.2 | 20.99 | 41.52 |
| Normal Lung 3 | 3157.31 | 22.69 | 23.69 |
| SW1353 resting 1 h | 3946.45 | 0.6 | 0 |
| SW1353 resting 6 h | 3263.46 | 0.12 | 0 |
| SW1353 resting 16 h | 2311.8 | 0 | 25.69 |
| SW1353 IL-1b (1 ng/) 1 h | 2686.83 | 0.16 | 0 |
| SW1353 IL-1b (1 ng/) 6 h | 3159.94 | 2 | 8.35 |
| SW1353 IL-1b (1 ng/) 16 h | 3557.88 | 0.62 | 0.93 |
| SW1353 FGF20 (1 ug/) | 3512.56 | 0.69 | 0.63 |
| 1 h | | | |
| SW1353 FGF20 (1 ug/) | 2510.14 | 0.06 | 0 |
| 16 h | | | |
| SW1353 FGF20 (5 ug/) | 3448.11 | 1.01 | 4.15 |
| 1 h | | | |
| SW1353 FGF20 (5 ug/) | 3598.07 | 0.64 | 6.73 |
| 6 h | | | |
| SW1353 FGF20 (5 ug/) | 3687.34 | 4.42 | 12.83 |
| 16 h | | | |
| SW1353 FGF20 (1 ug/) | 3569.19 | 0.17 | 4.31 |
| IL-1b (1 ng/) 6 h | | | |
| SW1353 FGF20 (1 ug/) | 3970.28 | 0.01 | 0 |
| IL-1b (1 ng/) 16 h | | | |
| SW1353 FGF20 (5 ug/) | 3011.45 | 0 | 0 |
| IL-1b (1 ng/) 1 h | | | |
| SW1353 FGF20 (5 ug/) | 3184.88 | 2.69 | 0 |
| IL-1b (1 ng/) 6 h | | | |
| THP-1 aCD40 (1 ug/) 1 h | 3545.99 | 0.07 | 42.81 |
| THP-1 aCD40 (1 ug/) 6 h | 2882.56 | 0.44 | 28.24 |
| THP-1 LPS (100 ng/) 1 h | 3131.64 | 0.74 | 77.98 |
| THP-1 LPS (100 ng/) 6 h | 2356.5 | 0.05 | 75.15 |
| CCD1070SK TNFa (5 | 2888.01 | 0.78 | 0 |
| ng/) 6 h | | | |
| CCD1070SK TNFa (5 | 3029 | 0.39 | 25.42 |
| ng/) 24 h | | | |
| CCD1070SK IL-1b (1 ng/) | 3307.91 | 5.76 | 0 |
| 24 h | | | |
| THP-1 resting | 3080.68 | 0.87 | 41.42 |
| THP-1 aCD40 (1 ug/) 24 h | 2032.49 | 0.82 | 2.16 |
| THP-1 LPS (100 ng/) 24 h | 1597.29 | 3.58 | 0 |
| CCD1070SK IL-1b (1 ng/) | 3026.56 | 3.51 | 1.09 |
| 6 h | | | |
| LC 18 hr | 2725.87 | 0.36 | 0 |
| LC-IL-! 18 hr | 1474.79 | 2.66 | 0 |
| Astrocyte_IL1B_1 hr_a | 3116.77 | 10.04 | 25.41 |
| Astrocyte_IL1B_6 hr_a | 3119.7 | 16.51 | 28.21 |
| Astrocyte_IL1B_24 hr_a | 3142.98 | 13.81 | 26.6 |
| SHSY 5Y Undifferentiated | 3166.23 | 14.61 | 47.94 |
| SHSY 5Y Differentiated | 2959.01 | 8.5 | 33.46 |
| LC 0 hr | 1880.7 | 0 | 1.17 |
| Normal Fetal Kidney | 2011.8 | 0 | 10.94 |
| Normal Liver | 2053.67 | 3.81 | 0 |
| Normal Fetal Liver | 3555.17 | 0 | 157.18 |
| Normal Fetal Lung | 4164.55 | 0 | 75.01 |
| Normal Salivary Gland | 3466.36 | 0 | 59.02 |
| Normal Fetal Skeletal | 2504.59 | 0 | 32.5 |
| Muscle | | | |
| Normal Thyroid | 3566.17 | 0 | 160.4 |
| Normal Trachea | 3596.15 | 0 | 100.33 |
| LC-IL-1 0 hr | 2560.23 | 0 | 2.58 |
| Heart pool | 3167.78 | 0 | 22.92 |
| Pituitary Pool | 2908.48 | 0 | 363.83 |
| Spleen Pool | 2068.62 | 0 | 36.16 |
| Stomach Pool | 2826.7 | 0 | 5169.42 |
| Testis Pool | 3348.7 | 0 | 36.46 |
| Thymus Pool | 2653.82 | 0 | 75.44 |
| Small Intestine - 5 donor | 3795.64 | 0 | 41.44 |
| pool | | | |
| Lymph node - 5 donor | 4339.52 | 0 | 108.49 |
| pool | | | |
| Kidney - 5 donor pool | 3347.34 | 0 | 12.82 |
| Jurkat Resting | 3779.74 | 0.48 | 32.89 |
| Jurkat CD3 (500 ng/ml) | 2459.46 | 1.68 | 89.9 |
| 6 hr A | | | |
| Jurkat CD3 (500 ng/ml) | 1897.6 | 0.08 | 85.79 |
| 24 hr A | | | |
| Jurkat CD3 | 1867.35 | 0.68 | 65.39 |
| (500 ng/ml) + CD28(1 ug/ml) | | | |
| 6 hr A | | | |
| Jurkat CD3 | 1574.07 | 0.35 | 103.43 |
| (500 ng/ml) + CD28(1 ug/ml) | | | |
| 24 hr A | | | |
| control (no treatment)_1 | 5400.28 | 2.01 | 46.65 |
| hr | | | |
| 10 ng/ml IL-1b_1 hr | 5250.91 | 1.51 | 63.47 |
| 10 ng/ml TNF-a_1 hr | 5668.06 | 2.55 | 57.06 |
| 200 uM BzATP_1 hr | 5619.87 | 0.31 | 47.95 |
| control (no treatment)_5 hr | 5630.13 | 1.02 | 73.66 |
| 10 ng/ml IL-1b_5 hr | 6332.12 | 10.52 | 55.94 |
| 10 ng/ml TNF-a_5 hr | 6070.17 | 6.37 | 52.01 |
| 200 uM BzATP_5 hr | 6425.22 | 2.39 | 155.96 |
| control (no treatment)_24 | 4825.87 | 3.5 | 56.3 |
| hr | | | |
| 10 ng/ml IL-1b_24 hr | 5349.28 | 9.73 | 55.52 |
| 10 ng/ml TNF-a_24 hr | 5672.31 | 4.97 | 85.13 |
| 200 uM BzATP_24 hr | 4814.1 | 0.51 | 38.39 |
| Alzheimer's disease | 1854.86 | 14.76 | 13.05 |
| B4951 | | | |
| Alzheimer's disease | 2540.02 | 19.95 | 20.79 |
| B4953 | | | |
| Alzheimer's disease | 1757.68 | 22.42 | 23.78 |
| B5018 | | | |
| Alzheimer's disease | 1491.9 | 18.37 | 10.32 |
| B5019 | | | |
| Alzheimer's disease | 2247.49 | 18.04 | 11.26 |
| B5086 | | | |
| Alzheimer's disease | 2150.92 | 19.76 | 23.52 |
| B5096 | | | |
| Alzheimer's disease | 732.56 | 15.93 | 0 |
| B5098 | | | |
| Alzheimer's disease | 1841.99 | 18.04 | 4.78 |
| B5129 | | | |
| Alzheimer's disease | 3233.87 | 21.56 | 24.49 |
| B5210 | | | |
| Control B4810 | 2987.22 | 21.85 | 18.41 |
| Control B4825 | 2903.91 | 18.74 | 18.18 |
| Control B4930 | 2287.12 | 22.69 | 22.12 |
| Control B4932 | 3424.98 | 20.12 | 17.99 |
| Control B5024 | 3859.32 | 22.42 | 27.93 |
| Control B5113 | 1897 | 17.87 | 11.6 |
| Control B5140 | 1901.93 | 18.88 | 11.57 |
| Control B5190 | 1284.69 | 15.26 | 0 |
| Control B5220 | 2225.75 | 18.7 | 20.26 |
| Control B5245 | 2119.39 | 21.5 | 15.57 |
| AH3 B3791 | 2202.74 | 19.73 | 18.98 |
| AH3 B3855 | 1849.89 | 17.93 | 17.84 |
| AH3 B3877 | 2144.15 | 17.01 | 14.36 |
| AH3 B3893 | 2103.76 | 16.34 | 38.69 |
| AH3 B3894 | 1820.82 | 17.31 | 24.16 |
| AH3 B3949 | 1607.86 | 23.9 | 19.16 |
| AH3 B4477 | 1602.58 | 20.32 | 19.91 |
| AH3 B4540 | 2260.92 | 22.79 | 24.33 |
| AH3 B4577 | 2142.42 | 22.59 | 15.4 |
| AH3 B4639 | 1550.43 | 21.89 | 22.7 |
| Schizophrenia | 2468.43 | 21.23 | 23.17 |
| hippocampus 683 | | | |
| Depression hippocampus | 1473.83 | 18 | 0 |
| 487 | | | |
| Depression hippocampus | 2481.12 | 10.69 | 19.51 |
| 600 | | | |
| Normal hippocampus | 2624.25 | 33.19 | 5.45 |
| 2407a | | | |
| Normal hippocampus | 2114.72 | 21.4 | 26.01 |
| 1042 | | | |
| Depression hippocampus | 1448.13 | 10.6 | 0 |
| 2767 | | | |
| Depression hippocampus | 1836.77 | 47.98 | 24.55 |
| 567 | | | |
| Control hippocampus | 2752.14 | 14.38 | 12.79 |
| 3175 | | | |
| Depression hippocampus | 1735.6 | 9.4 | 29.15 |
| 3096 | | | |
| Depression hippocampus | 2784.26 | 17.28 | 43.46 |
| 1491 | | | |
| Depression hippocampus | 2241.25 | 16.73 | 19.63 |
| 2540 | | | |
| Schizophrenia | 1923.26 | 16.97 | 25.74 |
| hippocampus 2798 | | | |
| Control hippocampus | 2605.59 | 14.97 | 17.73 |
| 1973 | | | |
| Normal hippocampus and | 2031.45 | 13.85 | 4.33 |
| amygdala 2601 | | | |
| Schizophrenia | 1621.43 | 21.03 | 0 |
| hippocampus 2785 | | | |
| Schizophrenia | 3271.31 | 39.54 | 13.79 |
| hippocampus 484 | | | |
| Normal hippocampus | 2806.68 | 21.69 | 43.9 |
| 2556 | | | |
| Depression hippocampus | 2705.56 | 15.83 | 27.65 |
| 1158 | | | |
| Control hippocampus 552 | 3378.83 | 21.65 | 42 |
| Schizophrenia | 2304.06 | 12.06 | 26.74 |
| hippocampus 1737 | | | |
| Normal hippocampus | 3335.3 | 17.54 | 32.32 |
| 1239 | | | |
| Normal hippocampus | 3068.38 | 14.64 | 20.08 |
| 1465 | | | |
| Normal hippocampus | 1323.77 | 4.6 | 0 |
| 3080 | | | |
| Normal hippocampus 738 | 4229.67 | 12.96 | 31.21 |
| Schizophrenia | 2067.82 | 12.78 | 24.47 |
| hippocampus 2586 | | | |
| Normal hippocampus | 3306.02 | 13.41 | 40.59 |
| 2551 | | | |
| Depression hippocampus | 2352.1 | 13.53 | 48.17 |
| 588 | | | |
| Depression hippocampus | 3291.7 | 13.05 | 70.51 |
| 529 | | | |
| Depression hippocampus | 1686.08 | 11.37 | 13.7 |
| and dentate gyrus | | | |
| Schizophrenia amygdala | 2136.57 | 0.55 | 12.87 |
| 2586 | | | |
| Normal substantia nigra | 1240.83 | 0.03 | 24.82 |
| 234 | | | |
| Normal substantia nigra | 1515.69 | 14.19 | 4.35 |
| 1065 | | | |
| Normal substantia nigra | 1341.77 | 0 | 4.92 |
| 3236 | | | |
| Normal substantia nigra | 3718.91 | 0.43 | 9.47 |
| 2551 | | | |
| Normal substantia nigra | 1003.1 | 0 | 16.45 |
| 1597 | | | |
| Control thalamus 552 | 948.15 | 0.02 | 27.84 |
| Control thalamus 566 | 1698.42 | 0 | 0 |
| Control thalamus 606 | 2625.59 | 0.31 | 0 |
| Control thalamus 738 | 2464.52 | 0 | 16.96 |
| Control thalamus 1065 | 2991.38 | 0 | 9.56 |
| Control thalamus 1092 | 2416.75 | 0 | 21.85 |
| Control thalamus 1597 | 2389.84 | 0 | 11.51 |
| Control thalamus 2253 | 1602.4 | 0 | 16.48 |
| Control thalamus 2551 | 3017.98 | 0 | 7.29 |
| Depression thalamus 588 | 2245.45 | 0.29 | 0 |
| Depression thalamus 600 | 1442.56 | 0 | 4.58 |
| Depression thalamus 721 | 1921.28 | 0 | 0 |
| Depression thalamus 728 | 3113.17 | 4.33 | 21.2 |
| Depression thalamus 759 | 2433.85 | 0 | 4.52 |
| Depression thalamus 881 | 2456.7 | 0 | 4.48 |
| Schizophrenia thalamus | 1952.08 | 0 | 11.27 |
| 477 | | | |
| Schizophrenia thalamus | 3553.95 | 0 | 22.29 |
| 532 | | | |
| Schizophrenia thalamus | 3798.02 | 0 | 8.69 |
| 683 | | | |
| Schizophrenia thalamus | 3260.82 | 0.03 | 11.47 |
| 544 | | | |
| Schizophrenia thalamus | 2246.95 | 0 | 5.39 |
| 1671 | | | |
| Schizophrenia thalamus | 1958.75 | 0.01 | 0 |
| 1737 | | | |
| Schizophrenia thalamus | 1953.64 | 0 | 0 |
| 2464 | | | |
| Schizophrenia thalamus | 3338.49 | 0 | 19.77 |
| 2586 | | | |
| Depression amygdala | 1936.43 | 0 | NA |
| 600 | | | |
| Depression amygdala | 2378.2 | 0 | 74.93 |
| 759 | | | |
| Depression anterior | 2808.48 | 0 | 28.2 |
| cingulate 759 | | | |
| Control amygdala 552 | 3675.87 | 0.36 | 7.78 |
| Control anterior cingulate | 3115.46 | 0.67 | 28.6 |
| 482 | | | |
| Depression anterior | 1964.87 | 0 | 0 |
| cingulate 721 | | | |
| Control amygdala 3175 | 2674.16 | 0.04 | 16.87 |
| Depression anterior | 2389.83 | 0.06 | 0 |
| cingulate 600 | | | |
| Depression anterior | 2629.92 | 0 | 10.04 |
| cingulate 588 | | | |
| Control anterior cingulate | 3605.03 | 0.79 | 21.36 |
| 3175 | | | |
| Control anterior cingulate | 2414.64 | 0 | NA |
| 606 | | | |
| Depression anterior | 2397.28 | 1.2 | 9.64 |
| cingulate 567 | | | |
| Depression amygdala | 3410.76 | 3.1 | 33.86 |
| 588 | | | |
| Control anterior cingulate | 2445.09 | 3.01 | 18.9 |
| 3080 | | | |
| Control anterior cingulate | 2520 | 5.45 | 22.26 |
| 2601 | | | |
| Control anterior cingulate | 3118.04 | 0.76 | 31.75 |
| 1042 | | | |
| Control anterior cingulate | 2913.66 | 1.45 | 34.73 |
| 3236 | | | |
| Control amygdala 1502 | 4253.4 | 9.62 | 40.86 |

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequence selected from the group consisting of SEQ ID NO: 2n, wherein n is an integer between 1 and 48.

2. An isolated polypeptide comprising an amino acid sequence selected from the group consisting of SEQ ID NO: 2n, wherein n is an integer between 1 and 48.

3. An isolated polypeptide comprising an amino acid sequence which is at least 95% identical to an amino acid sequence selected from the group consisting of SEQ ID NO: 2n, wherein n is an integer between 1 and 48.

4. An isolated polypeptide, wherein the polypeptide comprises an amino acid sequence comprising one or more conservative substitutions in the amino acid sequence selected from the group consisting of SEQ ID NO: 2n, wherein n is an integer between 1 and 48.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide,
thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator of activity or of latency or of predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO: 2n, wherein n is an integer between 1 and 48 or a biologically active fragment thereof.

20. An isolated nucleic acid molecule comprising a nucleic acid sequence selected from the group consisting of SEQ ID NO: 2n-1, wherein n is an integer between 1 and 48.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n-1, wherein n is an integer between 1 and 48.

23. An isolated nucleic acid molecule encoding the mature form of a polypeptide having an amino acid sequence selected from the group consisting of SEQ ID NO: 2n, wherein n is an integer between 1 and 48.

24. An isolated nucleic acid molecule comprising a nucleic acid selected from the group consisting of 2n-1, wherein n is an integer between 1 and 48.

25. The nucleic acid molecule of claim 20, wherein said nucleic acid molecule hybridizes under stringent conditions to the nucleotide sequence selected from the group consisting of SEQ ID NO: 2n-1, wherein n is an integer between 1 and 48, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO: 2n-1, wherein n is an integer between 1 and 48.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO: 2n-1, wherein n is an integer between 1 and 48.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.

46. An isolated polypeptide comprising an amino acid sequence at least 95% similar to SEQ ID NO: 6, wherein said amino acid sequence comprises at least one amino acid substitution, wherein said substitution is at the amino acid position selected from the group consisting of 171 when numbered in accordance with SEQ ID NO: 6.

47. An isolated nucleic acid molecule comprising an nucleic acid sequence at least 95% similar to SEQ ID NO: 5, wherein said nucleic acid sequence comprises at least one nucleic acid substitution, wherein said substitution is at the nucleic acid position selected from the group consisting of 566 when numbered in accordance with SEQ ID NO: 5.

48. An isolated polypeptide comprising an amino acid sequence at least 95% similar to SEQ ID NO: 12, wherein said amino acid sequence comprises at least one amino acid substitution, wherein said substitution is at the amino acid position selected from the group consisting of 49 when numbered in accordance with SEQ ID NO: 12.

49. An isolated nucleic acid molecule comprising an nucleic acid sequence at least 95% similar to SEQ ID NO: 11, wherein said nucleic acid sequence comprises at least one nucleic acid substitution, wherein said substitution is at the nucleic acid position selected from the group consisting of 148 when numbered in accordance with SEQ ID NO: 11.

50. An isolated polypeptide comprising an amino acid sequence at least 95% similar to SEQ ID NO: 34, wherein said amino acid sequence comprises at least one amino acid substitution, wherein said substitution is at the amino acid position selected from the group consisting of 13, 16, 68, 110, 128, 421, 479, or 496 when numbered in accordance with SEQ ID NO: 34.

51. An isolated nucleic acid molecule comprising an nucleic acid sequence at least 95% similar to SEQ ID NO: 33, wherein said nucleic acid sequence comprises at least one nucleic acid substitution, wherein said substitution is at the nucleic acid position selected from the group consisting of 77, 86, 242, 367, 421, 1301, 1475, or 1526 when numbered in accordance with SEQ ID NO: 33.

52. An isolated polypeptide comprising an amino acid sequence at least 95% similar to SEQ ID NO: 46, wherein said amino acid sequence comprises at least one amino acid substitution, wherein said substitution is at the amino acid position selected from the group consisting of 143, 219, 381, 401, or 587 when numbered in accordance with SEQ ID NO: 46.

53. An isolated nucleic acid molecule comprising an nucleic acid sequence at least 95% similar to SEQ ID NO: 45, wherein said nucleic acid sequence comprises at least one nucleic acid substitution, wherein said substitution is at the nucleic acid position selected from the group consisting of 437, 664, 1150, 1210, or 1770 when numbered in accordance with SEQ ID NO: 45.

54. An isolated polypeptide comprising an amino acid sequence at least 95% similar to SEQ ID NO: 68, wherein said amino acid sequence comprises at least one amino acid substitution, wherein said substitution is at the amino acid position selected from the group consisting of 479, 592, or 599 when numbered in accordance with SEQ ID NO: 68.

55. An isolated nucleic acid molecule comprising an nucleic acid sequence at least 95% similar to SEQ ID NO: 67, wherein said nucleic acid sequence comprises at least one nucleic acid substitution, wherein said substitution is at the nucleic acid position selected from the group consisting of 1466, 1805, or 1826 when numbered in accordance with SEQ ID NO: 67.

56. An isolated polypeptide comprising an amino acid sequence at least 95% similar to SEQ ID NO: 78, wherein said amino acid sequence comprises at least one amino acid substitution, wherein said substitution is at the amino acid position selected from the group consisting of 87, 119, 198, 222, or 261 when numbered in accordance with SEQ ID NO: 78.

57. An isolated nucleic acid molecule comprising an nucleic acid sequence at least 95% similar to SEQ ID NO: 77, wherein said nucleic acid sequence comprises at least one nucleic acid substitution, wherein said substitution is at the nucleic acid position selected from the group consisting of 259, 357, 593, 666, or 783 when numbered in accordance with SEQ ID NO: 77.

58. An isolated polypeptide comprising an amino acid sequence at least 95% similar to SEQ ID NO: 96, wherein said amino acid sequence comprises at least one amino acid substitution, wherein said substitution is at the amino acid position selected from the group consisting of 399 when numbered in accordance with SEQ ID NO: 96.

59. An isolated nucleic acid molecule comprising an nucleic acid sequence at least 95% similar to SEQ ID NO: 95, wherein said nucleic acid sequence comprises at least one nucleic acid substitution, wherein said substitution is at the nucleic acid position selected from the group consisting of 1458 when numbered in accordance with SEQ ID NO: 95.


SEQUENCE LISTING (173 sequences)

SEQ ID NO: 1 - DNA, 2343 bp, Homo sapiens
  CDS (36)..(1328)
gagcgctaacgtctttctgtctccccgcggtggtgatgacggtgaaaactgaggctgctaagggcaccctcacttactccaggatgaggggcatggtggcaattctcatcgctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgacttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttagagtgccgccttagacggaggcaggagtttcgttagaaagcggacgctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcttaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttgaaaaata

SEQ ID NO: 2 - protein, 431 aa, Homo sapiens
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 3 - DNA, 1315 bp, Homo sapiens
  CDS (14)..(1306)
caccggatccaccatgacggtgaaaactgaggctgctaagggcaccctcacttactccaggatgaggggcatggtggcaattctcatcgctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcgtttctatgctgctgaaatagccagtgccttgggctacctgcattcactgaacatcgtttatagagacttaaaaccagagaatattttgctagattcacagggacacattgtccttactgacttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctcctcgagggc

SEQ ID NO: 4 - protein, 431 aa, Homo sapiens; identical to SEQ ID NO: 2

SEQ ID NO: 5 - DNA, 1140 bp, Homo sapiens
  CDS (55)..(1131)
attggagtttggaagttcaggagcacaggagcacaggcccacgactgcagcgggatggaccagtactgcatcctgggccgcatcggggagggcgcccacggcatcgtcttcaaggccaagcacgtggagccgagggtgggctggcagtgtctgccttctatcctgcagactggcgagatagttgccctcaagaaggtggccctaaggcggttggaggacggcttccctaaccaggccctgcgggagattaaggctctgcaggagatggaggacaatcagtatgtggtacaactgaaggctgtgttcccacacggtggaggctttgtgctggcctttgagttcatgctgtcggatctggccgaggtggtgcgccatgcccagaggccactagcccaggcacaggtcaagagctacctgcagatgctgctcaagggtgtcgccttctgccatgccaacaacattgtacatcgggacctgaaacctgccaacctgctcatcagcgcctcaggccagctcaagatagcggactttggcctggctcgagtcttttccccagacggcagccacctctacacacaccaggtggccaccaggtggtaccgagcccccgagctcctgtatggtgcccgccagtatgaccagggcgtcgatctgtggtctgtgggctgcatcatgggggagctgttgaatgggtccccccttttcccgggcaagaacgatattgaacagctttgctatgtgcttcgcatcttgggcaccccaaaccctcaagtctggccggagctcactgagctgccggactacaacaagatctcctttaaggagcaggtgcccatgcccctggaggaggtgctgcctgacgtctctccccaggcattggatctgctgggtcaattccttctctaccctcctcaccagcgcatcgcagcttccaaggctctcctccatcagtacttcttcacagctcccctgcctgcccatccatctgagctgccgattcctcagcgtctagggggacctgcccccaaggcccatccagggcccccccacatccatgacttccacgtggaccggcctcttgaggagtcgctgttgaacccagagctgattcggcccttcatcctggaggggtgagaagtt

SEQ ID NO: 6 - protein, 359 aa, Homo sapiens
MDQYCILGRIGEGAHGIVFKAKHVEPRVGWQCLPSILQTGEIVALKKVALRRLEDGFPNQALREIKALQEMEDNQYVVQLKAVFPHGGGFVLAFEFMLSDLAEVVRHAQRPLAQAQVKSYLQMLLKGVAFCHANNIVHRDLKPANLLISASGQLKIADFGLARVFSPDGSHLYTHQVATRWYRAPELLYGARQYDQGVDLWSVGCIMGELLNGSPLFPGKNDIEQLCYVLRILGTPNPQVWPELTELPDYNKISFKEQVPMPLEEVLPDVSPQALDLLGQFLLYPPHQRIAASKALLHQYFFTAPLPAHPSELPIPQRLGGPAPKAHPGPPHIHDFHVDRPLEESLLNPELIRPFILEG

SEQ ID NO: 7 - DNA, 2078 bp, Homo sapiens
  CDS (238)..(1593)
caggtgctgcgtgacagcggagggctaggaaaaggcgcagtggggcccggagctgtcacccctgactcgacgcagcttccgttctcctggtgacgtggcctacaggaaccgccccggtggtcagctgccgcgctgttgctaggcaacagcgtgcgtgctcagatcagcgtggggtggaggagaagtggagtttggaagttcaggggcacaggggcacaggcccacgactgcagcgggatggaccagtactgcatcctgggccgcatcggggagggcgcccacggcatcgtcttcaaggccaagcacgtggagactggcgagataattgccctcaagaaggtggccctaaggcggttggaagacggcttccctaaccaggccctgcgggagattaaggctctgcaggagatggaggacaatcagtatgtggtacaactgaaggctgtgttcccacacggtggaggctttgtgctggcctttgagttcatgctgtcggatctggccgaggtggtgcgccatgcccagaggccactagcccaggcacaggtcaagagctacctgcagatgctgctcaagggtgtcgccttctgccatgccaacaacattgtacatcgggacctgaaacctgccaacctgctcatcagcgcctcaggccagctcaagatagcggactttggcctggctcgagtcttttccccagacggcagccgcctctacacacaccaggtggccaccaggtctgtgggctgcatcatgggggagctgttgaatgggtccccccttttcccgggcaagaacgatattgaacagctttgctatgtgcttcgcatcttgggcaccccaaaccctcaagtctggccggagctcactgagctgccggactacaacaagatctcctttaaggagcaggtgcccatgcccctggaggaggtgctgcctgacgtctctccccaggcattggatctgctgggtcaattccttctctaccctcctcaccagcgcatcgcagcttccaaggctctcctccatcagtacttcttcacagctcccctgcctgcccatccatctgagctgccgattcctcagcgtctagggggacctgcccccaaggcccatccagggcccccccacatccatgacttccacgtggaccggcctcttgagggagtcgctgttgaacccagagctgattcggcccttcatcctggaggggtgagaagttggccctggtcccgtctgcctgctcctcaggaccactcagtccacctgttcctctgccacctgcctggcttcaccctccaaggcctccccatggccacagtgggcccacaccacaccttgccccttagcccttgcgagggttggtctcgaggcagaggtcatgttcccagccaagagtatgagaacatccagtcgagcagaggagattcatggcctgtgctcggtgagccttaccttctgtgtgctactgacgtacccatcaggacagtgagctctgctgccagtcaaggcctgcatatgcagaatgacgatgcctgccttggtgctgcttcccccgagtgctgcctcctggtcaaggagaagtgcagagagtaaggtgtccttatgttggaaactcaagtggaaggaagatttggtttggttttattctcagagccattaaacactagttcagtatgtgagatatagattctaaaaacctcaggtggtctgcttagtctgtcttcttcctttctcaagggaaatggctaaggtggcattgtctcatggctctcgtttttggggtcatggggagggtagcaccagcatagccacttttgccctgagggactcctgtgtgcttcacatcactgagcactcatttagaagtgagggagacagaagtctaggcccagggatggctccagttggggatccagcaggagaccctctgcacatgaggctggtttaccaacatctactccctcaggatgagcgtgagccagaagcagctgtgtatttaaggaaacaagcgttcctggaattaatttataaatttaataaatcccaatataatcccaaaaaaaaaaaaaaaaaaa

SEQ ID NO: 8 - protein, 452 aa, Homo sapiens [sequence omitted]

SEQ ID NO: 9 - DNA, 958 bp, Homo sapiens
  CDS (99)..(944)
agccagagacaggacaccagaagagacaggagatcagagaccagaggaacagagaagaggccccagagcaaggcaaggaacggccaaggcaccaggacatggatgcagtggagccaggaggacgtggctgggccagcatgttggcgtgcaggctttggaaagccatcagcagggcgctgtttgcagagttcctggccacggggctgtatgtgttctttggcgtgggctcagtcatgcgctggcccacagcacttccctccgtgctacagattgccatcaccttcaacctggtcaccgccatggctgtgcaggtcacctggaaggccagcggggcccacgccaaccccgccgtgacgctggccttcctcgtaggctcccacatctctctgccccgtgctgtggcctatgtggctgcccagctggtgggggccacggtgggggctgctctgctttatggggtcatgccgggagacatccgagagacccttgggatcaacgtggtccggaacagtgtctcaactggccaggcggtggcagtggagctgcttctgaccctgcagctggtgctctgtgtcttcgcttccaccgacagccgtcagacatcaggctccccggccaccatgattgggatctctgtggcactgggccacctcattgggatccacttcactggctgctccatgaatccagcccgctccttcggccctgccatcatcattgggaagttcacagtccactgggtcttctgggtggggcccctgatgggagccctcctggcctcactgatctacaacttcgtcctgttccccgacaccaagaacctggcgcagcggctggctatcctcacaggcaccgtagaggtggggacaggggcaggggcaggggcggagcccctgaagaaggaatcccagccgggttcgggagccgtggagatggagagtgtgtgaaacagcctaac

SEQ ID NO: 10 - protein, 282 aa, Homo sapiens
MDAVEPGGRGWASMLACRLWKAISRALFAEFLATGLYVFFGVGSVMRWPTALPSVLQIAITFNLVTAMAVQVTWKASGAHANPAVTLAFLVGSHISLPRAVAYVAAQLVGATVGAALLYGVMPGDIRETLGINVVRNSVSTGQAVAVELLLTLQLVLCVFASTDSRQTSGSPATMIGISVALGHLIGIHFTGCSMNPARSFGPAIIIGKFTVHWVFWVGPLMGALLASLIYNFVLFPDTKNLAQRLAILTGTVEVGTGAGAGAEPLKKESQPGSGAVEMESV

SEQ ID NO: 11 - DNA, 1067 bp, Homo sapiens
  CDS (3)..(1046)
ctttggaacagaaccagtcaacagattattattatgaggaaaatgaaatgaatggcacttatgactacagtcaatatgaactgatctgtatcaaagaagatgtcagagaatttgcaaaagttttcctccctgtattcctcacaatagttttcgtcattggacttgcaggcaattccatggtagtggcaatttatgcctattacaagaaacagagaaccaaaacagatgtgtacatcctgaatttggctgtagcagatttactccttctattcactctgcctttttgggctgttaatgcagttcatgggtgggttttagggaaaataatgtgcaaaataacttcagccttgtacacactaaactttgtctctggaatgcagtttctggcttgtatcagcatagacagatatgtggcagtaactaaagtccccagccaatcaggagtgggaaaaccatgctggatcatctgtttctgtgtctggatggctgccatcttgctgagcataccccagctggttttttatacagtaaatgacaatgctaggtgcattcccattttcccccgctacctaggaacatcaatgaaagcattgattcaaatgctagagatctgcattggatttgtagtaccctttcttattatgggggtgtgctactttatcacggcaaggacactcatgaagatgccaaacattaaaatatctcgacccctaaaagttctgctcacagtcgttatagttttcattgtcactcaactgccttataacattgtcaagttctgccgagccatagacatcatctactctctgatcaccagctgcaacatgagcaaacgcatggacatcgccatccaagtcacagaaagcatcgcactctttcacagctgcctcaacccaatcctttatgtttttatgggagcatctttcaaaaactacgttatgaaagtggccaagaaatatgggtcctggagaagacagagacaaagtgtggaggagtttccttttgattctgagggtcctacagagccaaccagtacttttagcatttaaaggtaaaactgctctgcc

SEQ ID NO: 12 - protein, 348 aa, Homo sapiens
LEQNQSTDYYYEENEMNGTYDYSQYELICIKEDVREFAKVFLPVFLTIVFVIGLAGNSMVVAIYAYYKKQRTKTDVYILNLAVADLLLLFTLPFWAVNAVHGWVLGKIMCKITSALYTLNFVSGMQFLACISIDRYVAVTKVPSQSGVGKPCWIICFCVWMAAILLSIPQLVFYTVNDNARCIPIFPRYLGTSMKALIQMLEICIGFVVPFLIMGVCYFITARTLMKMPNIKISRPLKVLLTVVIVFIVTQLPYNIVKFCRAIDIIYSLITSCNMSKRMDIAIQVTESIALFHSCLNPILYVFMGASFKNYVMKVAKKYGSWRRQRQSVEEFPFDSEGPTEPTSTFSI

SEQ ID NO: 13 - DNA, 1953 bp, Homo sapiens
  CDS (16)..(1929)
gtctgagtcacagagatgggcaagatcgagaacaacgagagggtgatcctcaatgtcgggggcacccggcacgaaacctaccgcagcaccctcaagaccctgcctggaacacgcctggcccttcttgcctcctccgagcccccaggcgactgcttgaccacggcgggcgacaagctgcagccgtcgccgcctccactgtcgccgccgccgagagcgcccccgctgtcccccgggccaggcggctgcttcgagggcggcgcgggcaactgcagttcccgcggcggcagggccagcgaccatcccggtggcggccgcgagttcttcttcgaccggcacccgggcgtcttcgcctatgtgctcaattactaccgcaccggcaagctgcactgccccgcagacgtgtgcgggccgctcttcgaggaggagctggccttctggggcatcgacgagaccgacgtggagccctgctgctggatgacctaccggcagcaccgcgacgccgaggaggcgctggacatcttcgagacccccgacctcattggcggcgaccccggcgacgacgaggacctggcggccaagaggctgggcatcgaggacgcggcggggctcgggggcccggacggcaaatctggccgctggaggaggctgcagccccgcatgtgggccctcttcgaagacccctactcgtccagagccgccaggtttattgcttttgcttctttattcttcatcctggtttcaattacaactttttgcctggaaacacatgaagctttcaatattgttaaaaacaagacagaaccagtcatcaatggcacaagtgttgttctacagtatgaaattgaaacggatcctgccttgacgtatgtagaaggagtgtgtgtggtgtggtttacttttgaatttttagtccgtattgttttttcacccaataaacttgaattcatcaaaaatctcttgaatatcattgactttgtggccatcctacctttctacttagaggtgggactcagtgggctgtcatccaaagctgctaaagatgtgcttggcttcctcagggtggtaaggtttgtgaggatcctgagaattttcaagctcacccgccattttgtaggtctgagggtgcttggacatactcttcgagctagtactaatgaatttttgctgctgataattttcctggctctaggagttttgatatttgctaccatgatctactatgccgagagagtgggagctcaacctaacgacccttcagctagtgagcacacacagttcaaaaacattcccattgggttctggtgggctgtagtgaccatgactaccctgggttatggggatatgtacccccaaacatggtcaggcatgctggtgggagccctgtgtgctctggctggagtgctgacaatagccatgccagtgcctgtcattgtcaataattttggaatgtactactccttggcaatggcaaagcagaaacttccaaggaaaagaaagaagcacatccctcctgctcctcaggcaagctcacctactttttgcaagacagaattaaatatggcctgcaatagtacacagagtgacacatgtctgggcaaagacaatcgacttctggaacataacagatcagtgttatcaggtgacgacagtacaggaagtgagccgccactatcacccccagaaaggctccccatcagacgctctagtaccagagacaaaaacagaagaggggaaacatgtttcctactgacgacaggtgattacacgtgtgcttctgatggagggatcaggaaaggttatgaaaaatcccgaagcttaaacaacatagcgggcttggcaggcaatgctctgaggctctctccagtaacatcaccctacaactctccttgtcctctgaggcgctctcgatctcccatcccatctatcttgtaaaccaaacaaccaaactgcatc

SEQ ID NO: 14 - protein, 638 aa, Homo sapiens
MGKIENNERVILNVGGTRHETYRSTLKTLPGTRLALLASSEPPGDCLTTAGDKLQPSPPPLSPPPRAPPLSPGPGGCFEGGAGNCSSRGGRASDHPGGGREFFFDRHPGVFAYVLNYYRTGKLHCPADVCGPLFEEELAFWGIDETDVEPCCWMTYRQHRDAEEALDIFETPDLIGGDPGDDEDLAAKRLGIEDAAGLGGPDGKSGRWRRLQPRMWALFEDPYSSRAARFIAFASLFFILVSITTFCLETHEAFNIVKNKTEPVINGTSVVLQYEIETDPALTYVEGVCVVWFTFEFLVRIVFSPNKLEFIKNLLNIIDFVAILPFYLEVGLSGLSSKAAKDVLGFLRVVRFVRILRIFKLTRHFVGLRVLGHTLRASTNEFLLLIIFLALGVLIFATMIYYAERVGAQPNDPSASEHTQFKNIPIGFWWAVVTMTTLGYGDMYPQTWSGMLVGALCALAGVLTIAMPVPVIVNNFGMYYSLAMAKQKLPRKRKKHIPPAPQASSPTFCKTELNMACNSTQSDTCLGKDNRLLEHNRSVLSGDDSTGSEPPLSPPERLPIRRSSTRDKNRRGETCFLLTTGDYTCASDGGIRKGYEKSRSLNNIAGLAGNALRLSPVTSPYNSPCPLRRSRSPIPSIL

SEQ ID NO: 15 - DNA, 607 bp, Homo sapiens
  CDS (13)..(603)
agatttcccaccatgggcaagatcgagaacaacgagagggtgatcctcaatgtcgggggcacccggcacgaaacctaccgcagcaccctcaagaccctgcctggaacacgcctggcccttcttgcctcctccgagcccccaggcgactgcttgaccacagcgggcaactgcagttcccgcggcggcagggccagcgaccatcccggtggcggccgcgagttcttcttcgaccggcatccgggcgtcttcgcctatgtgctcaattactaccgcaccggcaagctgcactgtcccgcagacgtgtgcgggccgctcttcgaggaggagctggccttctggggcatcgacgagaccgacgtggagccctgctgctggatgacctaccggcagcaccgcgacgccgaggaggcgctggacatcttcgagacccccgacctcattggcggcgaccccggcgacgacgaggacctggcggccaagaggctgggcatcgaggacgcggcggggctcgggggccccgacggcaaatctggccgctggaggaggctgcagccccgcatgtgggccctcttcgaagacccctactcgtccagagccgccaggctcg

SEQ ID NO: 16 - protein, 197 aa, Homo sapiens
MGKIENNERVILNVGGTRHETYRSTLKTLPGTRLALLASSEPPGDCLTTAGNCSSRGGRASDHPGGGREFFFDRHPGVFAYVLNYYRTGKLHCPADVCGPLFEEELAFWGIDETDVEPCCWMTYRQHRDAEEALDIFETPDLIGGDPGDDEDLAAKRLGIEDAAGLGGPDGKSGRWRRLQPRMWALFEDPYSSRAAR

SEQ ID NO: 17 - DNA, 1815 bp, Homo sapiens
  CDS (13)..(1809)
agatctcccaccatgggcaagatcgagaacaacgagagggtgatcctcaatgtcgggggcacccggcacgaaacctaccgcagcaccctcaagaccctgcctggaacacgcctggcccttcttgcctcctccgagcccccaggcaactgcagttcccgcggcggcagggccagcgaccatcccggtggcggccgcgagttcttcttcgaccggcacccgggcgtcttcgcctatgtgctcaattactaccgcaccggcaagctgcactgccccgcagacgtgtgcgggccgctcttcgaggaggagctggccttctggggcatcgacgagaccgacgtggagccctgctgctggatgacctaccggcagcaccgcgacgccgaggaggcgctggacatcttcgagacccccgacctcattggcggcgaccccggcgacgacgaggacctggcggccaagaggctgggcatcgaggacgcggcggggctcgggggccccgacggcaaatctggccgctggaggaggctgcagccccgcatgtgggccctcttcgaagacccctactcgtccagagccgccaggtttattgcttttgcttctttattcttcatcctggtttcaattacaactttttgcctggaaacacatgaagctttcaatattgttaaaaacaagacagaaccagtcatcaatggcacaagtgttgttctacagtatgaaattgaaacggatcctgccttgacgtatgtagaaggagtgtgtgtggtgtggtttacttttgaatttttagtccgtattgttttttcacccaataaacttgaattcatcaaaaatctcttgaatatcattgactttgtggccatcctacctttctacttagaggtgggactcagtgggctgtcatccaaagctgctaaagatgtgcttggcttcctcagggtggtaaggtttgtgaggatcctgagaattttcaagctcacccgccattttgtaggtctgagggtgcttggacatactcttcgagctagtactaatgaatttttgctgctgataattttcctggctctaggagttttgatatttgctaccatgatctactatgccgagagagtgggagctcaacctaacgacccttcagctagtgagcacacacagttcaaaaacattcccattgggttctggtgggctgtagtgaccatgactaccctgggttatgaggatacgtacccccaaacatggtcaggcatgctggtgggagccctgtgtgctctggctggagtgctgacaatagccatgccagtgcctgtcattgtcaataattttggaatgtactactccttggcaatggcaaagcagaaacttccaaggaaaagaaagaagcacatccctcctgctcctcaggcaagctcacctactttttgcaagacagaattaaatatggcctgcaatagtacacagagtgacacatgtctgggcaaagacaatcgacttctggaacataacagatcagtgttatcaggtgacgacagtacaggaagtgagccgccactatcacccccagaaaggctccccatcagacgctctagtaccagagacaaaaacagaagaggggaaacatgtttcctactgacgacaggtgattacacgtgtgcttctgatggagggatcaggaaaggatatgaaaaatcccgaagcttaaacaacatagcgggcttggcaggcaatgctctgaggctctctccagtaacatcaccctacaactctccttgtcctctgaggcgctctcgatctcccatcccatctatcttgctcgag

SEQ ID NO: 18 - protein, 599 aa, Homo sapiens
MGKIENNERVILNVGGTRHETYRSTLKTLPGTRLALLASSEPPGNCSSRGGRASDHPGGGREFFFDRHPGVFAYVLNYYRTGKLHCPADVCGPLFEEELAFWGIDETDVEPCCWMTYRQHRDAEEALDIFETPDLIGGDPGDDEDLAAKRLGIEDAAGLGGPDGKSGRWRRLQPRMWALFEDPYSSRAARFIAFASLFFILVSITTFCLETHEAFNIVKNKTEPVINGTSVVLQYEIETDPALTYVEGVCVVWFTFEFLVRIVFSPNKLEFIKNLLNIIDFVAILPFYLEVGLSGLSSKAAKDVLGFLRVVRFVRILRIFKLTRHFVGLRVLGHTLRASTNEFLLLIIFLALGVLIFATMIYYAERVGAQPNDPSASEHTQFKNIPIGFWWAVVTMTTLGYEDTYPQTWSGMLVGALCALAGVLTIAMPVPVIVNNFGMYYSLAMAKQKLPRKRKKHIPPAPQASSPTFCKTELNMACNSTQSDTCLGKDNRLLEHNRSVLSGDDSTGSEPPLSPPERLPIRRSSTRDKNRRGETCFLLTTGDYTCASDGGIRKGYEKSRSLNNIAGLAGNALRLSPVTSPYNSPCPLRRSRSPIPSIL

SEQ ID NO: 19 - DNA, 3028 bp, Homo sapiens
  CDS (22)..(1860)
agtcatgtctgagtcacagagatgggcaagatcgagaacaacgagagggtgatcctcaatgtcgggggcacccggcacgaaacctaccgcagcaccctcaagaccctgcctggaacacgcctggcccttcttgcctcctccgagcccccaggcgactgcttgaccacggcgggcgacaagctgcagccgtcgccgcctccactgtcgccgccgccgagagcgcccccgctgtcccccgggccaggcggctgcttcgagggcggcgcgggcaactgcagttcccgcggcggcagggccagcgaccatcccggtggcggccgcgagttcttcttcgaccggcacccgggcgtcttcgcctatgtgctcaattactaccgcaccggcaagctgcactgccccgcagacgtgtgcgggccgctcttcgaggaggagctggccttctggggcatcgacgagaccgacgtggagccctgctgctggatgacctaccggcagcaccgcgacgccgaggaggcgctggacatcttcgagacccccgacctcattggcggcgaccccggcgacgacgaggacctggcggccaagaggctgggcatcgaggacgcggcggggctcgggggccccgacggcaaatctggccgctggaggaggctgcagccccgcatgtgggccctcttcgaagacccctactcgtccagagccgccaggtttattgcttttgcttctttattcttcatcctggtttcaattacaactttttgcctggaaacacatgaagctttcaatattgttaaaaacaagacagaaccagtcatcaatggcacaagtgttgttctacagtatgaaattgaaacggatcctgccttgacgtatgtagaaggagtgtgtgtggtgtggtttacttttgaatttttagtccgtattgttttttcacccaataaacttgaattcatcaaaaatctcttgaatatcattgactttgtggccatcctacctttctacttagaggtgggactcagtgggctgtcatccaaagctgctaaagatgtgcttggcttcctcagggtggtaaggtttgtgaggatcctgagaattttcaagctcacccgccattttgtaggtctgagggtgcttggacatactcttcgagctagtactaatgaatttttgctgctgataattttcctggctctaggagttttgatatttgctaccatgatctactatgccgagagagtgggagctcaacctaacgacccttcagctagtgagcacacacagttcaaaaacattcccattgggttctggtgggctgtagtgaccatgactaccctgggttatggggatatgtacccccaaacatggtcaggcatgctggtgggagccctgtgtgctctggctggagtgctgacaatagccatgccagtgcctgtcattgtcaataattttggaatgtactactccttggcaatggcaaagcagaaacttccaaggaaaagaaagaagcacatccctcctgctcctcaggcaagctcacctactttttgcaagacagaattaaatatggcctgcaatagtacacagagtgacacatgtctgggcaaagacaatcgacttctggaacataacagatcagtgttatcaggtgacgacagtacaggaagtgagccgccactatcacccccagaaaggctccccatcagacgctctagtaccagagacaaaaacagaagaggggaaacatgtttcctactgacgacaggtgattacacgtgtgcttctgatggagggatcaggaaagataactgcaaagaggttgtcattactggttacacgcaagccgaggccagatctcttacttaatgacttgggggaaggcacaaaacatgagagaaagtgttgtacagaatttatcatggattattgactgctgagaaagggacagtggaatttagccatacaaaggactatactggaaacagacttctgctgctgaatgtgccctgatgtgaccaggttgcacttggaagagatcctccgcgtcttcatgaggcacttaaagcttataaaagaactgcggctggaactcatctggtgctccccatgagagtgctctgcttgtagactggccagtgtccatgaaacaactgtaaataccaacatgtgtgcatgggtcaacagtcttggccatttctcatcaaaagaagccaaattcatgatcaacatctctgaagtttcaagtaaggcccacacttctttgaattactcttcatgggcccacattaggttgtgctgtgaattacttaaggcagtgatactgatgtagtatagttttgtcttaatttcccttatttctacttctttggttgaatctatgaacttgattgtataattttcttataaattactgatgtaatcagcttgtcaattatgttgtgaaattgttagtattcatttatcaaaaatgacctatgtttagtcacatatttgtttagttctgggaaattgttatagcttaaatggaactcaccaacattattcatagtttaagtcttttatcattattacctcaattataaatattacaaaaacataattctggcaatgagagtatttttttattcaatgatcaaggagcaatgtcagtatatagtagaatatcaattaaattatatcctaaaatgtatattttgcataaaagagatattctttaatcaattacttttttgtgagttttgtggcgaatgaagcttgtacgtgtctttaaaactgttgtagatgaaactgtataagatttttacatcttgcttaatcaatattttcagagtctattagttcccctgggattctgaatataacatatagcctattataaatccctgtatcgtggaccttttgtgaacatttcaaggcgcatgcacaaccttgatgataaccagtggaaatgtaactaactgaaatgaagaataaaaggcaaatgagctggggataaacttgaatgttatctgattaaattactcaaattatt

SEQ ID NO: 20 - protein, 613 aa, Homo sapiens
MGKIENNERVILNVGGTRHETYRSTLKTLPGTRLALLASSEPPGDCLTTAGDKLQPSPPPLSPPPRAPPLSPGPGGCFEGGAGNCSSRGGRASDHPGGGREFFFDRHPGVFAYVLNYYRTGKLHCPADVCGPLFEEELAFWGIDETDVEPCCWMTYRQHRDAEEALDIFETPDLIGGDPGDDEDLAAKRLGIEDAAGLGGPDGKSGRWRRLQPRMWALFEDPYSSRAARFIAFASLFFILVSITTFCLETHEAFNIVKNKTEPVINGTSVVLQYEIETDPALTYVEGVCVVWFTFEFLVRIVFSPNKLEFIKNLLNIIDFVAILPFYLEVGLSGLSSKAAKDVLGFLRVVRFVRILRIFKLTRHFVGLRVLGHTLRASTNEFLLLIIFLALGVLIFATMIYYAERVGAQPNDPSASEHTQFKNIPIGFWWAVVTMTTLGYGDMYPQTWSGMLVGALCALAGVLTIAMPVPVIVNNFGMYYSLAMAKQKLPRKRKKHIPPAPQASSPTFCKTELNMACNSTQSDTCLGKDNRLLEHNRSVLSGDDSTGSEPPLSPPERLPIRRSSTRDKNRRGETCFLLTTGDYTCASDGGIRKDNCKEVVITGYTQAEARSLT

SEQ ID NO: 21 - DNA, 1193 bp, Homo sapiens
  CDS (105)..(1142)
tgctccctgtttcattaaaacctagagagatgtaatcagtaagcaagaaggaaaaagggaaattcacaaagtaactttttgtgtctgtttctttttaacccagcatggagagaaaatttatgtccttgcaaccatccatctccgtatcagaaatggaaccaaatggcaccttcagcaataacaacagcaggaactgcacaattgaaaacttcaagagagaatttttcccaattgtatatctgataatatttttctggggagtcttgggaaatgggttgtccatatatgttttcctgcagccttataagaagtccacatctgtgaacgttttcatgctaaatctggccatttcagatctcctgttcataagcacgcttcccttcagggctgactattatcttagaggctccaattggatatttggagacctggcctgcaggattatgtcttattccttgtatgtcaacatgtacagcagtatttatttcctgaccgtgctgagtgttgtgcgtttcctggcaatggttcacccctttcggcttctgcatgtcaccagcatcaggagtgcctggatcctctgtgggatcatatggatccttatcatggcttcctcaataatgctcctggacagtggctctgagcagaacggcagtgtcacatcatgcttagagctgaatctctataaaattgctaagctgcagaccatgaactatattgccttggtggtgggctgcctgctgccatttttcacactcagcatctgttatctgctgatcattcgggttctgttaaaagtggaggtcccagaatcggggctgcgggtttctcacaggaaggcactgaccaccatcatcatcaccttgatcatcttcttcttgtgtttcctgccctatcacacactgaggaccgtccacttgacgacatggaaagtgggtttatgcaaagacagactgcataaagctttggttatcacactggccttggcagcagccaatgcctgcttcaatcctctgctctattactttgctggggagaattttaaggacagactaaagtctgcactcagaaaaggccatccacagaaggcaaagacaaagtgtgttttccctgttagtgtgtggttgagaaaggaaacaagagtataaggagctcttagatgagacctgttcttgtatccttgtgtccatcttcat

SEQ ID NO: 22 - protein, 346 aa, Homo sapiens
MERKFMSLQPSISVSEMEPNGTFSNNNSRNCTIENFKREFFPIVYLIIFFWGVLGNGLSIYVFLQPYKKSTSVNVFMLNLAISDLLFISTLPFRADYYLRGSNWIFGDLACRIMSYSLYVNMYSSIYFLTVLSVVRFLAMVHPFRLLHVTSIRSAWILCGIIWILIMASSIMLLDSGSEQNGSVTSCLELNLYKIAKLQTMNYIALVVGCLLPFFTLSICYLLIIRVLLKVEVPESGLRVSHRKALTTIIITLIIFFLCFLPYHTLRTVHLTTWKVGLCKDRLHKALVITLALAAANACFNPLLYYFAGENFKDRLKSALRKGHPQKAKTKCVFPVSVWLRKETRV

SEQ ID NO: 23 - DNA, 1260 bp, Homo sapiens
  CDS (105)..(1142)
tgctccctgtttcattaaaacctagagagatgtaatcagtaagcaagaaggaaaaagggaaattcacaaagtaactttttgtgtctgtttctttttaacccagcatggagagaaaatttatgtccttgcaaccatccatctccgtatcagaaatggaaccaaatggcaccttcagcaataacaacagcaggaactgcacaattgaaaacttcaagagagaatttttcccaattgtatatctgataatatttttctggggagtcttgggaaatgggttgtccatatatgttttcctgcagccttataagaagtccacatctgtgaacgttttcatgctaaatctggccatttcagatctcctgttcataagcacgcttcccttcagggctgactattatcttagaggctccaattggatatttggagacctggcctgcaggattatgtcttattccttgtatgtcaacatgtacagcagtatttatttcctgaccgtgctgagtgttgtgcgtttcctggcaatggttcacccctttcggcttctgcatgtcaccagcatcaggagtgcctggatcctctgtgggatcatatggatccttatcatggcttcctcaataatgctcctggacagtggctctgagcagaacggcagtgtcacatcatgcttagagctgaatctctataaaattgctaagctgcagaccatgaactatattgccttggtggtgggctgcctgctgccatttttcacactcagcatctgttatctgctgatcattcgggttctgttaaaagtggaggtcccagaatcggggctgcgggtttctcacaggaaggcactgaccaccatcatcatcaccttgatcatcttcttcttgtgtttcctgccctatcacacactgaggaccgtccacttgacgacatggaaagtgggtttatgcaaagacagactgcataaagctttggttatcacactggccttggcagcagccaatgcctgcttcaatcctctgctctattactttgctggggagaattttaaggacagactaaagtctgcactcagaaaaggccatccacagaaggcaaagacaaagtgtgttttccctgttagtgtgtggttgagaaaggaaacaagagtataaggagctcttagatgagacctgttcttgtatccttgtgtccatcttcattcactcatagtctccaaatgactttgtatttacatcactcccaacaaatgttgattcttaatattta

SEQ ID NO: 24 - protein, 346 aa, Homo sapiens; identical to SEQ ID NO: 22

SEQ ID NO: 25 - DNA, 1358 bp, Homo sapiens
  CDS (5)..(1348)
agtgatggatgttaacagcagcggccacccggacctctacgggcgcctctgctctttcctcctgccggaggtggggggcaggctgcccgacctgagccccgacggtggcgccgaaccggtcgcggtctccgggacgccgcatctgctgagcgaggtgacggccagcccggcgcccacctgggacgcaaccccgggcaatgcctccggccgcggggagcaaatcaatcaagaaagggccgagaaagttgtgatcggctctgtcctgacgctcatctctctgtctgcgatcgcgggcaactgcctggtggtaatctctgtgtgcttcgtcaagaagctccgccagccctccaactacctcatcgtgtccatggcgctggccaacctctcggtggccatggcggtcatgcccttcatcagtgtcaccgacctcatcgggggcaagtggatctttggacactttttctgtaacgtcttctccgtgaatgtcatgtgctgcacggcctggatcttgaccttgtacgtgatcagcatcgacagggaccttgggatcatgaagcctctcacgtaccctatgaggcagaaggggaaatgcatgacgaagatgattctttctgtctgccttctttccgcctttgtcactttacctaccatttttggtcgggctcagaatgtaaacgatgataaggtgtgcttggtcaatcaagactttggctacacgatttactccccgcatttggcagcatttatccccatgtgcgtcatgcttttcatgtactatcagatttacaaggccgccaggaaaagcgcggccaaacacaggttacctggcttccctcgagtggagccagacagcgtagtcaccctgaatggcacagtgaagttccaggaggtggaagagtgtgcaaacctttcgagactcctcaagcatgaaaggaaaaatatctccatctttaagcggaaacagaaagcagcgactaccttggggatcatcgtctgggcctccaccatgtgctggccgccctttttcctcctgacagccagacccttctgtctatggcactgcccttctgtctatggcactgcctgcagctgcatcccactgtgggtggagaggatatttccatggctgggctatgcaaactctctcattaacccttttatttatgccttcttcaactgggacctgaggaccacctattgcagccggctccagtgccagtaccagaatatcaaccagacactctcagctgcaggcatgcatgaagccctgaagcttgctgagaggccagagagacctgagtttgtcctacaaaactctgactactgtagaaaaaaaagtcatgattcatgactgaaag

SEQ ID NO: 26 - protein, 448 aa, Homo sapiens
MDVNSSGHPDLYGRLCSFLLPEVGGRLPDLSPDGGAEPVAVSGTPHLLSEVTASPAPTWDATPGNASGRGEQINQERAEKVVIGSVLTLISLSAIAGNCLVVISVCFVKKLRQPSNYLIVSMALANLSVAMAVMPFISVTDLIGGKWIFGHFFCNVFSVNVMCCTAWILTLYVISIDRDLGIMKPLTYPMRQKGKCMTKMILSVCLLSAFVTLPTIFGRAQNVNDDKVCLVNQDFGYTIYSPHLAAFIPMCVMLFMYYQIYKAARKSAAKHRLPGFPRVEPDSVVTLNGTVKFQEVEECANLSRLLKHERKNISIFKRKQKAATTLGIIVWASTMCWPPFFLLTARPFCLWHCPSVYGTACSCIPLWVERIFPWLGYANSLINPFIYAFFNWDLRTTYCSRLQCQYQNINQTLSAAGMHEALKLAERPERPEFVLQNSDYCRKKSHDS

SEQ ID NO: 27 - DNA, 1173 bp, Homo sapiens
  CDS (55)..(1023)
aatatagccggtcctgtccccgtgttaactggagtgctgaagcgctggaacgaaatgcggggcctctggtgcgagaagggggtgcaggtgctgctgacgacggtgggcgccttcgccgccttcggcctcatgaccatcgccatcagcactgactactggctctacacggggcaacaagagcgaaaatctgtctcaaaaaataaaagaagtaagaaggaccccggcggcctcacgcactcgggcctctggaggatctgctgcctggaagggttgaaaagaggcgtctgcgtgaagatcaatcatttcccggaggacacggactacgaccacgacagcgcggagtatctactccgtacggtccgggcctccagcatcttccccatccttagcgccatcctgctgctgctcgggggtgtgtgcgtggcggcctcccgcgtctacaagtccaagaggaacatcattctgggcgcagggatcctgttcgtggcagcaggtctgagcaacatcatcggcgtgatcgtgtacatctccgccaacgcgggcgagccgggccgaagcagagccaagaaaaaccactactcgtacggctggtccttctacttcggcgggctgtcgttcatcctggccgaggtgataggcgtgctggccgtcaacatctacatcgagccagccgaggcgcactgccagtctcggagcgggaccgcggggggtcgtccggcttcctcacgctgcacaacgccttccccaaggaggcgggcggcggcgtcacggtcacgttcacccggccgcccgccccgcccgcgccacgccaccccggccaacaccaactccacggacatctccatgtacacgctcagccgcgacccctccaagggcagcccccattccaatgccaccacccccacccccactagcctcaaggataggaaaaactcatttgtgtccataaaaataaaggtaaaaaagaaaaaaagaaatatatatatatatatatatacgctcaacaggaaaaccacgcctgtgtaggggcgcggcgggggagccgaggggcgtgtccggggcgcgtgcgggcgcgcgtgcatcgaggctgccggggtcgggggcgcccccgctttcccccgtgagcgcgctggagactgctgggcccgccccacgcccaccctccccgccccc

SEQ ID NO: 28 - protein, 323 aa, Homo sapiens [sequence omitted]

SEQ ID NO: 29 - DNA, 1192 bp, Homo sapiens
  CDS (69)..(1097)
tgctgagggtgcatttatgtttcagaaccaccgggaggaactgggccattctaacacccgttgctaccatgctggccacccgcctctccagacccctgtcacggctcccaggaaaaaccctaagtgcctgtgatagagaaaatggagcaaggcgcccactattgcttggttctacttcctttatcccgattggccgtcggacttatgccagtgcggcggagccggtgagtggaaaagctgtcctggtcacaggctgtgactctggatttgggttctcattggccaagcatctgcattcaaaaggcttccttgtgtttgctggctgcttgatgaaggacaaaggccatgatggggtcaaggagctggacagcctaaacagtgaccgattgagaaccgtccagctcaatgtctgcagcagcgaagaggtggagaaagtggtggagattgtccgctcgagcctgaaggaccctgagaaaggtatgtggggcctcgttaacaatgccggcatctcaacgttcggggaggtggagttcaccagcctggagacctacaagcaggtggcagaagtgaacctttggggcacagtgcggatgacgaaatcctttctccccctcatccgaagggccaaaggtcgcgtcgtcaatatcagcagcatgctgggccgcatggccaacccggcccgctccccgtactgcatcaccaagttcggggtagaggctttctcggactgcctgcgctatgagatgtaccccctgggcgtgaaggtcagcgtggtggagcccggcaacttcatcgctgccaccagcctttacagccctgagagcattcaggccatcgccaagaagatgtgggaggagctgcctgaggtcgtgcgcaaggactacggcaagaagtactttgatgaaaagatcgccaagatggagacctactgcagcagtggctccacagacacgtcccctgtcatcgatgctgtcacacacgccctgaccgccaccaccccctacacccgctaccaccccatggactactactggtggctgcgaatgcagatcatgacccacttgcctggagccatctccgacatgatctacatccgctgaagagtctcgctgtggcctctgtcagggatccctggtggaaggggaggggagggaggaacccatatagtcaactcttgattatccacgtgtgg

SEQ ID NO: 30 - protein, 343 aa, Homo sapiens
MLATRLSRPLSRLPGKTLSACDRENGARRPLLLGSTSFIPIGRRTYASAAEPVSGKAVLVTGCDSGFGFSLAKHLHSKGFLVFAGCLMKDKGHDGVKELDSLNSDRLRTVQLNVCSSEEVEKVVEIVRSSLKDPEKGMWGLVNNAGISTFGEVEFTSLETYKQVAEVNLWGTVRMTKSFLPLIRRAKGRVVNISSMLGRMANPARSPYCITKFGVEAFSDCLRYEMYPLGVKVSVVEPGNFIAATSLYSPESIQAIAKKMWEELPEVVRKDYGKKYFDEKIAKMETYCSSGSTDTSPVIDAVTHALTATTPYTRYHPMDYYWWLRMQIMTHLPGAISDMIYIR

SEQ ID NO: 31 - DNA, 1166 bp, Homo sapiens
  CDS (69)..(1097)
tgctgagggtgcatttatgtttcagaaccaccgggaggaactgggccattctaacacccgttgctaccatgctggccacccgcctctccagacccctgtcacggctcccaggaaaaaccctaagtgcctgtgatagagaaaatggagcaagacgcccactattgcttggttctacttcctttatcccgattggccgtcggacttatgccagtgcggcggagccggttggcagcaaagctgtcctggtcacaggctgtgactctggatttgggttctcattggccaagcatctgcattcaaaaggcttccttgtgtttgctggctgcttgatgaaggacaaaggccatgatggggtcaaggagctggacagcctaaacagtgaccgattgagaaccgtccagctcaatgtctgcagcagcgaagaggtggagaaagtggtggagattgtccgctcgagcctgaaggaccctgagaaaggcatgtggggcctcgttaacaatgccggcatctcaacgttcggggaggtggagttcaccagcctggagacctacaagcaggtggcagaagtgaacctttggggcacagtgcggatgacgaaatcctttctccccctcatccgaagggccaaaggccgcgtcgtcaatatcagcagcatgctgggccgcatggccaacccggcccgctccccgtactgcatcaccaagttcggggtagaggctttctcggactgcctgcgctatgagatgtaccccctgggcgtgaaggtcagcgtggtggagcccggcaacttcatcgctgccaccagcctttacagccctgagagcattcaggccatcgccaagaagatgtgggaggagctgcctgaggtcgtgcgcaaggactacggcaagaagtactttgatgaaaagatcgccaagatggagacctactgcagcagtggctccacagacacgtcccctgtcatcgatgctgtcacacacgccctgaccgccaccaccccctacacccgctaccaccccatggactactactggtggctgcgaatgcagatcatgacccacttgcctggagccatctccgacatgatctacatccgctgaagagtctcgctgtggcctctgtcagggattcctggtggaaggggaggggagggaggaacccatata

SEQ ID NO: 32 - protein, 343 aa, Homo sapiens
MLATRLSRPLSRLPGKTLSACDRENGARRPLLLGSTSFIPIGRRTYASAAEPVGSKAVLVTGCDSGFGFSLAKHLHSKGFLVFAGCLMKDKGHDGVKELDSLNSDRLRTVQLNVCSSEEVEKVVEIVRSSLKDPEKGMWGLVNNAGISTFGEVEFTSLETYKQVAEVNLWGTVRMTKSFLPLIRRAKGRVVNISSMLGRMANPARSPYCITKFGVEAFSDCLRYEMYPLGVKVSVVEPGNFIAATSLYSPESIQAIAKKMWEELPEVVRKDYGKKYFDEKIAKMETYCSSGSTDTSPVIDAVTHALTATTPYTRYHPMDYYWWLRMQIMTHLPGAISDMIYIR

SEQ ID NO: 33 - DNA, 3168 bp, Homo sapiens
  CDS (39)..(1610)
cacaagacctggaattgacaggactcccaactagtacaatgacagaagataaggtcactgggaccctggttttcactgtcatcactgctgtgctgggttccttccagtttggatatgacattggtgtgatcaatgcacctcaacaggtaataatatctcactatagacatgttttgggtgttccactggatgaccgaaaagctatcaacaactatgttatcaacagtacagatgaactgcccacaatctcatactcaatgaacccaaaaccaaccccttgggctgaggaagagactgtggcagctgctcaactaatcaccatgctctggtccctgtctgtatccagctttgcagttggtggaatgactgcatcattctttggtgggtggcttggggacacacttggaagaatcaaagccatgttagtagcaaacattctgtcattagttggagctctcttgatggggttttcaaaattgggaccatctcatatacttataattgctggaagaagcatatcaggactatattgtgggctaatttcaggcctggttcctatgtatatcggtgaaattgctccaaccgctctcaggggagcacttggcacttttcatcagctggccatcgtcacgggcattcttattagtcagattattggtcttgaatttatcttgggcaattatgatctgtggcacatcctgcttggcctgtctggtgtgcgagccatccttcagtctctgctactctttttctgtccagaaagccccagatacctttacatcaagttagatgaggaagtcaaagcaaaacaaagcttgaaaagactcagaggatatgatgatgtcaccaaagatattaatgaaatgagaaaagaaagagaagaagcatcgagtgagcagaaagtctctataattcagctcttcaccaattccagctaccgacagcctattctagtggcactgatgctgcatgtggctcagcaattttccggaatcaatggcattttttactactcaaccagcatttttcagacggctggtatcagcaaacctgtttatgcaaccattggagttggcgctgtaaacatggttttcactgctgtctctgtattccttgtggagaaggcagggcgacgttctctctttctaattggaatgagtgggatgtttgtttgtgccatcttcatgtcagtgggacttgtgctgctgaataagttctcttggatgagttatgtgagcatgatagccatcttcctctttgtcagcttctttgaaattgggccaggcccgatcccctggttcatggtggctgagtttttcagtcaaggaccacgtcctgctgctttagcaatagctgcattcagcaattggacctgcaatttcattgtagctctgtgtttccagtacattgcggacttctgtggaccttatgtgtttttcctctttgctggagtgctcctggcctttaccctgttcacattttttaaagttccagaaaccaaaggaaagtcttttgaggaaattgctgcagaattccaaaagaagagtggctcagcccacaggccaaaagctgctgtagaaatgaaattcctaggagctacagagactgtgtaaaaaaaaaaccctgctttttgacatgaacagaaacaataagggaaccgtctgtttttaaatgatgattccttgagcattttatatccacatctttaagtattgttttatttttatgtgctctcatcagaaatgtcatcaaatattaccaaaaaagtatttttttaagttagagaatatatttttgatggtaagactgtaattaagtaaaccaaaaaggctagtttattttgttacactaaagggcaggtggttctaatatttttagctctgttctttataacaaggttcttctaaaattgaagagatttcaacatatcatttttttaacacataactagaaacctgaggatgcaacaaatatttatatatttgaatatcattaaattggaattttcttacccatatatcttatgttaaaggagatatggctagtggcaataagttccatgttaaaatagacaactcttccatttattgcactcagcttttttcttgagtactagaatttgtattttgcttaaaattttacttttgttctgtattttcatgtggaatggattatagagtatactaaaaaatgtctatagagaaaaactttcatttttggtaggcttatcaaaatctttcagcactcagaaaagaaaaccattttagttcctttatttaatggccaaatggtttttgcaagatttaacactaaaaaggtttcacctgatcatatagcgtgggttatcagttaacattaacatctattataaaaccatgttgattcccttctggtacaatcctttgagttatagtttgctttgctttttaattgaggacagcctggttttcacatacactcaaacaatcatgagtcagacatttggtatattacctcaaattcctaataagtttgatcaaatctaatgtaagaaaatttgaagtaaaggattgatcactttgttaaaaatattttctgaattattatgtctcaaaataagttgaaaaggtagggtttgaggattcctgagtgtgggcttctgaaacttcataaatgttcagcttcagacttttatcaaaatccctatttaattttcctggaaagactgattgttttatggtgtgttcctaacataaaataatcgtctcctttgacatttccttctttgtcttagctgtatacagattctagccaaactattctatggccattactaacacgcattgtacactatctatctgcctttacctacataggcaaattggaaatacacagatgattaaacagactttagcttacagtcaattttacaattatggaaatatagttctgatgggtcccaaaagcttagcagggtgctaacgtatctctaggctgttttctccaccaactggagcactgatcaatccttcttatgtttgctttaatgtgtattgaagaaaagcactttttaaaaagtactctttaagagtgaaataattaaaaaccactgaacatttgctttgttttctaaagttgttcacatatatgtaatttagcagtccaaagaacaagaaattgtttcttttc

SEQ ID NO: 34 - protein, 524 aa, Homo sapiens
MTEDKVTGTLVFTVITAVLGSFQFGYDIGVINAPQQVIISHYRHVLGVPLDDRKAINNYVINSTDELPTISYSMNPKPTPWAEEETVAAAQLITMLWSLSVSSFAVGGMTASFFGGWLGDTLGRIKAMLVANILSLVGALLMGFSKLGPSHILIIAGRSISGLYCGLISGLVPMYIGEIAPTALRGALGTFHQLAIVTGILISQIIGLEFILGNYDLWHILLGLSGVRAILQSLLLFFCPESPRYLYIKLDEEVKAKQSLKRLRGYDDVTKDINEMRKEREEASSEQKVSIIQLFTNSSYRQPILVALMLHVAQQFSGINGIFYYSTSIFQTAGISKPVYATIGVGAVNMVFTAVSVFLVEKAGRRSLFLIGMSGMFVCAIFMSVGLVLLNKFSWMSYVSMIAIFLFVSFFEIGPGPIPWFMVAEFFSQGPRPAALAIAAFSNWTCNFIVALCFQYIADFCGPYVFFLFAGVLLAFTLFTFFKVPETKGKSFEEIAAEFQKKSGSAHRPKAAVEMKFLGATETV

SEQ ID NO: 35 - DNA, 1587 bp, Homo sapiens
  CDS (1)..(1587)
ggatccaccatgacagaagataaggtcactgggaccctggttttcactgtcatcactgctgtgctgggttccttccagtttggatatgacattggtgtgatcaatgcacctcaacaggtaataatatctcactatagacatgttttgggtgttccactggatgaccgaaaagctatcaacaactatgttatcaacagtacagatgaactgcccacaatctcatactcaatgaacccaaaaccaaccccttgggctgaggaagagactgtggcagctgctcaactaatcaccatgctctggtccctgtctgtatccagctttgcagttggtggaatgattgcatcattctttggtgggtggcttggggacacacttggaagaatcaaagccatgttagtagcaaacattctgtcattagttggagctctcttgatggggttttcaaaattgggaccatctcatatacttataattgctggaagaagcatatcaggactatattgtgggctaatttcaggcctggttcctatgtatatcggtgaaattgctccaaccgctctcaggggagcacttggcacttttcatcagctggccatcgtcacgggcattcttattagtcagattattggtcttgaatttatcttgggcaattatgatctgtggcacatcctgcttggcctgtctggtgtgcgagccatccttcagtctctgctactctttttctgtccagaaagccccagatacctttacatcaagttagatgaggaagtcaaagcaaaacaaatcttgaaaagactcagaggatatgatgatgtcaccaaagatattaatgaaatgagaaaagaaagagaagaagcatcgagtgagcagaaagtctctataattcagctcttcaccaattccagctaccgacagcctattctagtggcactgatgctgcatgtggctcagcaattttccggaatcaatggcattttttactactcaaccagcatttttcagacggctggtatcagcaaacctgtttatgcaaccattggagttggcgctgtaaacatggttttcactgctgtctctgtattccttgtggagaaggcagggcgacgttctctctttctaattggaatgagtgggatgtttgtttgtgccatcttcatgtcagtgggacttgtgctgctgaataagttctcttggatgagttatgtgagcatgatagccatcttcctctttgtcagcttctttgaaattgggccaggcccgatcccctggttcatggtggctgagtttttcagtcaaggaccacgtcctgctgctttagcaatagctgcattcagcaattggacctgcaatttcattgtagctctgtgtttccagtacattgcggacttctgtggaccttatgtgtttttcctctttgctggagtgctcctggcctttaccctgtttacattttttaaagttccagaaaccaaaggaaagtcttttgaggaaattgctgcagaattccaaaagaagagtggctcagcccacaggccaaaagctgctgtagaaatgaaattcctaggagctacagagactgtgctcgag

SEQ ID NO: 36 - protein, 529 aa, Homo sapiens
GSTMTEDKVTGTLVFTVITAVLGSFQFGYDIGVINAPQQVIISHYRHVLGVPLDDRKAINNYVINSTDELPTISYSMNPKPTPWAEEETVAAAQLITMLWSLSVSSFAVGGMIASFFGGWLGDTLGRIKAMLVANILSLVGALLMGFSKLGPSHILIIAGRSISGLYCGLISGLVPMYIGEIAPTALRGALGTFHQLAIVTGILISQIIGLEFILGNYDLWHILLGLSGVRAILQSLLLFFCPESPRYLYIKLDEEVKAKQILKRLRGYDDVTKDINEMRKEREEASSEQKVSIIQLFTNSSYRQPILVALMLHVAQQFSGINGIFYYSTSIFQTAGISKPVYATIGVGAVNMVFTAVSVFLVEKAGRRSLFLIGMSGMFVCAIFMSVGLVLLNKFSWMSYVSMIAIFLFVSFFEIGPGPIPWFMVAEFFSQGPRPAALAIAAFSNWTCNFIVALCFQYIADFCGPYVFFLFAGVLLAFTLFTFFKVPETKGKSFEEIAAEFQKKSGSAHRPKAAVEMKFLGATETVLE

SEQ ID NO: 37 - DNA, 1587 bp, Homo sapiens
  CDS (1)..(1587)
ggatccaccatgacagaagataaggtcactgggaccctggttttcactgtcatcactgctgtgctgggttccttccagtttggatatgacattggtgtgatcaatgcacctcaacaggtaataatatctcactatagacatgttttgggtgttccactggatgaccgaaaagctatcaacaactatgttatcaacagtacagatgaactgcccacaatctcatactcaatgaacccaaaaccaaccccttgggctgaggaagagactgtggcagctgctcaactaatcaccatgctctggtccctgtctgtatccagctttgcagttggtggaatgattgcatcattctttggtgggtggcttggggacacacttggaagaatcaaagccatgttagtagcaaacattctgtcattagttggagctctcttgatggggttttcaaaattgggaccatctcatatacttataattgctggaagaagcatatcaggactatattgtgggctaatttcaggcctggttcctatgtatatcggtgaaattgctccaaccgctctcaggggagcacttggcacttttcatcagctggccatcgtcacgggcattcttattagtcagattattggtcttgaatttatcttgggcaattatgatctgtggcacatcctgcttggcctgtctggtgtgcgagccatccttcagtctctgctactctttttctgtccagaaagccccagatacctttacatcaagttagatgaggaagtcaaagcaaaacaaagcttgaaaagactcagaggatatgatgatgtcaccaaagatattaatgaaatgagaaaagaaagagaagaagcatcgagtgagcagaaagtctctataattcagctcttcaccaattccagctaccgacagcctattctagtggcactgatgctgcatgtggctcagcaattttccggaatcaatggcattttttactactcaaccagcatttttcagacggctggtatcagcaaacctgtttatgcaaccattggagttggcgctgtaaacatggttttcactgctgtctctgtattccttgtggagaaggcagggcgacgttctctctttctaattggaatgagtgggatgtttgtttgtgccatcttcatgtcagtgggacttgtgctgctgaataagttctcttggatgagttatgtgagcatgatagccatcttcctctttgtcagcttctttgaaattgggccaggcccgatcccctggttcatggtggctgagtttttcagtcaaggaccacgtcctgctgctttagcaatagctgcattcagcaattggacctgcaatttcattgtagctctgtgtttccagtacattgcggacttctgtggaccttatgtgtttttcctctttgctggagtgctcctggcctttaccctgtttacattttttaaagttccagaaaccaaaggaaagtcttttgaggaaattgctgcagaattccaaaagaagagtggctcagcccacaggccaaaagctgctgtagaaatgaaattcctaggagctacagagactgtgctcgag

SEQ ID NO: 38 - protein, 529 aa, Homo sapiens
GSTMTEDKVTGTLVFTVITAVLGSFQFGYDIGVINAPQQVIISHYRHVLGVPLDDRKAINNYVINSTDELPTISYSMNPKPTPWAEEETVAAAQLITMLWSLSVSSFAVGGMIASFFGGWLGDTLGRIKAMLVANILSLVGALLMGFSKLGPSHILIIAGRSISGLYCGLISGLVPMYIGEIAPTALRGALGTFHQLAIVTGILISQIIGLEFILGNYDLWHILLGLSGVRAILQSLLLFFCPESPRYLYIKLDEEVKAKQSLKRLRGYDDVTKDINEMRKEREEASSEQKVSIIQLFTNSSYRQPILVALMLHVAQQFSGINGIFYYSTSIFQTAGISKPVYATIGVGAVNMVFTAVSVFLVEKAGRRSLFLIGMSGMFVCAIFMSVGLVLLNKFSWMSYVSMIAIFLFVSFFEIGPGPIPWFMVAEFFSQGPRPAALAIAAFSNWTCNFIVALCFQYIADFCGPYVFFLFAGVLLAFTLFTFFKVPETKGKSFEEIAAEFQKKSGSAHRPKAAVEMKFLGATETVLE

SEQ ID NO: 39 - DNA, 1664 bp, Homo sapiens
  CDS (39)..(1610)
cacaagacctggaattgacaggactcccaactagtacaatgacagaagataaggtcactgggaccctggttttcactgtcatcactgctgtgctgggttccttccagtttggatatgacattggtgtgatcaatgcacctcaacaggtaataatatctcactatagacatgttttgggtgttccactggatgaccgaaaagctatcaacaactatgttatcaacagtacagatgaactgcccacaatctcatactcaatgaacccaaaaccaaccccttgggctgaggaagagactgtggcagctgctcaactaatcaccatgctctggtccctgtctgtatccagctttgcagttggtggaatgactgcatcattctttggtgggtggcttggggacacacttggaagaatcaaagccatgttagtagcaaacattctgtcattagttggagctctcttgatggggttttcaaaattgggaccatctcatatacttataattgctggaagaagcatatcaggactatattgtgggctaatttcaggcctggttcctatgtatatcggtgaaattgctccaaccgctctcaggggagcacttggcacttttcatcagctggccatcgtcacgggcattcttattagtcagattattggtcttgaatttatcttgggcaattatgatctgtggcacatcctgcttggcctgtctggtgtgcgagccatccttcagtctctgctactctttttctgtccagaaagccccagatacctttacatcaagttagatgaggaagtcaaagcaaaacaaagcttgaaaagactcagaggatatgatgatgtcaccaaagatattaatgaaatgagaaaagaaagagaagaagcatcgagtgagcagaaagtctctataattcagctcttcaccaattccagctaccgacagcctattctagtggcactgatgctgcatgtggctcagcaattttccggaatcaatggcattttttactactcaaccagcatttttcagacggctggtatcagcaaacctgtttatgcaaccattggagttggcgctgtaaacatggttttcactgctgtctctgtattccttgtggagaaggcagggcgacgttctctctttctaattggaatgagtgggatgtttgtttgtgccatcttcatgtcagtgggacttgtgctgctgaataagttctcttggatgagttatgtgagcatgatagccatcttcctctttgtcagcttctttgaaattgggccaggcccgatcccctggtttatggtggctgagtttttcagtcaaggaccacgtcctgctgctttagcaatagctgcattcagcaattggacctgcaatttcattgtagctctgtgtttccagtacattgcggacttctgtggaccttatgtgtttttcctctttgctggagtgctcctggcctttaccctgtttacattttttaaagttccagaaaccaaaggaaagtcttttgaggaaattgctgcagaattccaaaagaagagtggctcagcccacaggccaaaagctgctgtagaaatgaaattcctaggagctacagagactgtgtaaaaaaaaaaccctgctttttggcatgaacagaaacaataagggaaccgtctg

SEQ ID NO: 40 - protein, 524 aa, Homo sapiens; identical to SEQ ID NO: 34

SEQ ID NO: 41 - DNA, 1587 bp, Homo sapiens
  CDS (10)..(1581)
ggatccaccatgacagaagataaggtcactgggaccctggttttcactgtcatcactgctgtgctgggttccttccagtttggatatgacattggtgtgatcaatgcacctcaacaggtaataatatctcactatagacatgttttgggtgttccactggatgaccgaaaagctatcaacaactatgttatcaacagtacagatgaactgcccacaatctcatactcaatgaacccaaaaccaaccccttgggctgaggaagagactgtggcagctgctcaactaatcaccatgctctggtccctgtctgtatccagctttgcagttggtggaatgactgcatcattctttggtgggtggcttggggacacacttggaagaatcaaagccatgttagtagcaaacattctgtcattagttggagctctcttgatggggttttcaaaattgggaccatctcatatacttataattgctggaagaagcatatcaggactatattgtgggctaatttcaggcctggttcctatgtatatcggtgaaattgctccaaccgctctcaggggagcacttggcacttttcatcagctggccatcgtcacgggcattcttattagtcagattattggtcttgaatttatcttgggcaattatgatctgtggcacatcctgcttggcctgtctggtgtgcgagccatccttcagtctctgctactctttttctgtccagaaagccccagatacctttacatcaagttagatgaggaagtcaaagcaaaacaaagcttgaaaagactcagaggatatgatgatgtcaccaaagatattaatgaaatgagaaaagaaagagaagaagcatcgagtgagcagaaagtctctataattcagctcttcaccaattccagctaccgacagcctattctagtggcactgatgctgcatgtggctcagcaattttccggaatcaatggcattttttactactcaaccagcatttttcagacggctggtatcagcaaacctgtttatgcaaccattggagttggcgctgtaaacatggttttcactgctgtctctgtattccttgtggagaaggcagggcgacgttctctctttctaattggaatgagtgggatgtttgtttgtgccatcttcatgtcagtgggacttgtgctgctgaataagttctcttggatgagttatgtgagcatgatagccatcttcctctttgtcagcttctttgaaattgggccaggcccgatcccctggtttatggtggctgagtttttcagtcaaggaccacgtcctgctgctttagcaatagctgcattcagcaattggacctgcaatttcattgtagctctgtgtttccagtacattgcggacttctgtggaccttatgtgtttttcctctttgctggagtgctcctggcctttaccctgtttacattttttaaagttccagaaaccaaaggaaagtcttttgaggaaattgctgcagaattccaaaagaagagtggctcagcccacaggccaaaagctgctgtagaaatgaaattcctaggagctacagagactgtgctcgag

SEQ ID NO: 42 - protein, 524 aa, Homo sapiens; identical to SEQ ID NO: 34

SEQ ID NO: 43 - DNA, 1473 bp, Homo sapiens
  CDS (7)..(1467)
ggatccactgtcatcactgctgtgctgggttccttccagtttggatatgacattggtgtgatcaatgcacctcaacaggtaataatatctcactatagacatgttttgggtgttccactggatgaccgaaaagctatcaacaactatgttatcaacagtacagatgaactgcccacaatctcatactcaatgaacccaaaaccaaccccttgggctgaggaagagactgtggcagctgctcaactaatcaccatgctctggtccctgtctgtatccagctttgcagttggtggaatgactgcatcattctttggtgggtggcttggggacacacttggaagaatcaaagccatgttagtagcaaacattctgtcattagttggagctctcttgatggggttttcaaaattgggaccatctcatatacttataattgctggaagaagcatatcaggactatattgtgggctaatttcaggcctggttcctatgtatatcggtgaaattgctccaaccgctctcaggggagcacttggcacttttcatcagctggccatcgtcacgggcattcttattagtcagattattggtcttgaatttatcttgggcaattatgatctgtggcacatcctgcttggcctgtctggtgtgcgagccatccttcagtctctgctactctttttctgtccagaaagccccagatacctttacatcaagttagatgaggaagtcaaagcaaaacaaagcttgaaaagactcagaggatatgatgatgtcaccaaagatattaatgaaatgagaaaagaaagagaagaagcatcgagtgagcagaaagtctctataattcagctcttcaccaattccagctaccgacagcctattctagtggcactgatgctgcatgtggctcagcaattttccggaatcaatggcattttttactactcaaccagcatttttcagacggctggtatcagcaaacctgtttatgcaaccattggagttggcgctgtaaacatggttttcactgctgtctctgtattccttgtggagaaggcagggcgacgttctctctttctaattggaatgagtgggatgtttgtttgtgccatcttcatgtcagtgggacttgtgctgctgaataagttctcttggatgagttatgtgagcatgatagccatcttcctctttgtcagcttctttgaaattgggccaggcccgatcccctggtttatggtggctgagtttttcagtcaaggaccacgtcctgctgctttagcaatagctgcattcagcaattggacctgcaatttcattgtagctctgtgtttccagtacattgcggacttctgtggaccttatgtgtttttcctctttgctggagtgctcctggcctttaccctgtttacattttttaaagttccagaaaccaaaggaaagtcttttgaggaaattgctgcagaattcctcgag

SEQ ID NO: 44 - protein, 487 aa, Homo sapiens
TVITAVLGSFQFGYDIGVINAPQQVIISHYRHVLGVPLDDRKAINNYVINSTDELPTISYSMNPKPTPWAEEETVAAAQLITMLWSLSVSSFAVGGMTASFFGGWLGDTLGRIKAMLVANILSLVGALLMGFSKLGPSHILIIAGRSISGLYCGLISGLVPMYIGEIAPTALRGALGTFHQLAIVTGILISQIIGLEFILGNYDLWHILLGLSGVRAILQSLLLFFCPESPRYLYIKLDEEVKAKQSLKRLRGYDDVTKDINEMRKEREEASSEQKVSIIQLFTNSSYRQPILVALMLHVAQQFSGINGIFYYSTSIFQTAGISKPVYATIGVGAVNMVFTAVSVFLVEKAGRRSLFLIGMSGMFVCAIFMSVGLVLLNKFSWMSYVSMIAIFLFVSFFEIGPGPIPWFMVAEFFSQGPRPAALAIAAFSNWTCNFIVALCFQYIADFCGPYVFFLFAGVLLAFTLFTFFKVPETKGKSFEEIAAEF

SEQ ID NO: 45 - DNA, 2038 bp, Homo sapiens
  CDS (10)..(1923)
gttttcagaatgattttattcaagcaagcaacttatttcatttccttgtttgctacagtttcctgtggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtttggttgcttcttgaaagatagtgttacaggaaccctgccaaaagtacatcgaacaggtgcagtttctggacattccttgaagcaatgtggtcatcaaataagtgcttgccatcgagacatttataaaggagttgatatgagaggagtcaattttaatgtgtctaaggttagcagtgttgaagaatgccaaaaaaggtgcaccaataacattcgctgccagtttttttcatatgccacgcaaacatttcacaaggcagagtaccggaacaattgcctattaaagtacagtcccggaggaacacctaccgctataaaggtgctgagtaacgtggaatctggattctcactgaagccctgtgccctttcagaaattggttgccacatgaacatcttccagcatcttgcgttctcagatgtggatgttgccaggtttctcactccagatgcttttgtgtgtcggaccatctgcacctatcaccccaactgcctcttctttacattctatacaaatgtatggaaaatcgagtcacaaagaaatgtttgtcttcttaaaacatctgaaagtggcacaccaagttcctctactcctcaagaaaacaccatatctggatatagccttttaacctgcaaaagaactttacctgaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtgtaagtgtttcttaagattatctatggatggttctccaactaggattgcgtatgggacacaagggagctctggttactctttgagattgtgtaacactggggacaacgctgtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactgaattccaaaaaccaatatgcctaccttccaaaggtgacacaagcacaatttataccaactgttgggtaaccggatggggcttctcgaaggagaaaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaagggagattcaggtggtcccttagtttgcaaacacaacggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatgaagagtggcatcttctttgcatcctaag

SEQ ID NO: 46 - protein, 638 aa, Homo sapiens
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARFLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNAVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 47 - DNA, 1869 bp, Homo sapiens
  CDS (1)..(1869)
ggatccggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtttggttgcttcttgaaagatagtgttacaggaaccctgccaaaagtacatcgaacaggtgcagtttctggacattccttgaagcaatgtggtcatcaaataagtgcttgccatcgagacatttataaaggagttgatatgagaggagtcaattttaatgtgtctaaggttagcagtgttgaagaatgccaaaaaaggtgcaccagtaacattcgctgccagtttttttcatatgccacgcaaacatttcacaaggcagagtaccggaacaattgcctattaaagtacagtcccggaggaacacctaccgctataaaggtgctgagtaacgtggaatctggattctcactgaagccctgtgccctttcagaaattggttgccacatgaacatcttccagcatcttgcgttctcagatgtggatgttgccaggtttctcactccagatgcttttgtgtgtcggaccatctgcacctatcaccccaactgcctcttctttacattctatacaaatgtatggaaaatcgagtcacaaagaaatgtttgtcttcttaaaacatctgaaagtggcacaccaagttcctctactcctcaagaaaacaccatatctggatatagccttttaacctgcaaaagaactttacctgaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtgtaagtgtttcttaagattatctatggatggttctccaactaggattgcgtatgggacacaagggagctctggttactctttgagattgtgtaacactggggacaacgctgtctgcacaacaaaaacaagcacacgcattgttggaggaacaaactcttcttggggagagtggccctggcaggtgagcctgcaggtgaagctgacagctcagaggcatctgtgtggagggtcactcataggacaccagtgggtcctcactgctgcccactgctttgatgggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactgaattccaaaaaccaatatgcctaccttccaaaggtgacacaagcacaatttataccaactgttgggtaaccggatggggcttctcgaaggagaaaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcactcgag

SEQ ID NO: 48 - protein, 623 aa, Homo sapiens
GSGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTSNIRCQFFSYATQTFHKAEYRNNCLLKYSPGGTPTAIKVLSNVESGFSLKPCALSEIGCHMNIFQHLAFSDVDVARFLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNAVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPALE

SEQ ID NO: 49 - DNA, 2245 bp, Homo sapiens
  CDS (72)..(1985)
agaacagcttgaagaccgttcatttttaagtgacaagagactcacctccaagaagcaattgtgttttcagaatgattttattcaagcaagcaacttatttcatttccttgtttgctacagtttcctgtggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtttggttgcttcttgaaagatagtgttacaggaaccctgccaaaagtacatcgaacaggtgcagtttctggacattccttgaagcaatgtggtcatcaaataagtgcttgccatcgagacatttataaaggagttgatatgagaggagtcaattttaatgtgtctaaggttagcagtgttgaagaatgccaaaaaaggtgcaccaataacattcgctgccagtttttttcatatgccacgcaaacatttcacaaggcagagtaccggaacaattgcctattaaagtacagtcccggaggaacacctaccgctataaaggtgctgagtaacgtggaatctggattctcactgaagccctgtgccctttcagaaattggttgccacatgaacatcttccagcatcttgcgttctcagatgtggatgttgccagggttctcactccagatgcttttgtgtgtcggaccatctgcacctatcaccccaactgcctcttctttacattctatacaaatgtatggaaaatcgagtcacaaagaaatgtttgtcttcttaaaacatctgaaagtggcacaccaagttcctctactcctcaagaaaacaccatatctggatatagccttttaacctgcaaaagaactttacctgaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtgtaagtgtttcttaagattatctatggatggttctccaactaggattgcgtatgggacacaagggagctctggttactctttgagattgtgtaacactggggacaactctgtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactgaattccaaaaaccaatatgcctaccttccaaaggtgacacaagcacaatttataccaactgttgggtaaccggatggggcttctcgaaggagaaaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaagggagattcaggtggtcccttagtttgcaaacacaacggaatgtggcgtttggtgggcatcacaagctggggtgaaggctgtgcccgcagggagcaacctggtgtctacaccaaagtcgctgagtacatggactggattttagagaaaacacagagcagtgatggaaaagctcagatgcagtcaccagcatgagaagcagtccagagtctaggcaatttttacaacctgagttcaagtcaaattctgagcctggggggtcctcatctgcaaagcatggagagtggcatcttctttgcatcctaaggacgaaagacacagtgcactcagagctgctgaggacaatgtctgctgaagcccgctttcagcacgccgtaaccaggggctgacaatgcgaggtcgcaactgagatctccatgactgtgtgttgtgaaataaaatggtgaaagatc

SEQ ID NO: 50 - protein, 638 aa, Homo sapiens
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 51 - DNA, 1869 bp, Homo sapiens
  CDS (7)..(1863)
ggatccggatgtctgactcaactctatgaaaacgccttcttcagaggtggggatgtagcttccatgtacaccccaaatgcccaatactgccagatgaggtgcacattccacccaaggtgtttgctattcagttttcttccagcaagttcaatcaatgacatggagaaaaggtttggttgcttcttgaaagatagtgttacaggaaccctgccaaaagtacatcgaacaggtgcagtttctggacattccttgaagcaatgtggtcatcaaataagtgcttgccatcgagacatttataaaggagttgatatgagaggagtcaattttaatgtgtctaaggttagcagtgttgaagaatgccaaaaaaggtgcaccagtaacattcgctgccagtttttttcatatgccacgcaaacatttcacaaggcagagtaccggaacaattgcctattaaagtacagtcccggaggaacacctaccgctataaaggtgctgagtaacgtggaatctggattctcactgaagccctgtgccctttcagaaattggttgccacatgaacatcttccagcatcttgcgttctcagatgtggatgttgccaggtttctcactccagatgcttttgtgtgtcggaccatctgcacctatcaccccaactgcctcttctttacattctatacaaatgtatggaaaatcgagtcacaaagaaatgtttgtcttcttaaaacatctgaaagtggcacaccaagttcctctactcctcaagaaaacaccatatctggatatagccttttaacctgcaaaagaactttacctgaaccctgccattctaaaatttacccgggagttgactttggaggagaagaattgaatgtgacttttgttaaaggagtgaatgtttgccaagagacttgcacaaagatgattcgctgtcagtttttcacttattctttactcccagaagactgtaaggaagagaagtgtaagtgtttcttaagattatctatggatggttctccaactaggattgcgtatgggacacaagggagctctggttactctttgagattgtgtaacactggggacaacgctgtctgcacaacaaaaacaagcacacgcattgttggaggaacaaactcttcttggggagagtggccctggcaggtgagcctgcaggtgaagctgacagctcagaggcacctgtgtggagggtcactcataggacaccagtgggtcctcactgctgcccactgctttgatgggcttcccctgcaggatgtttggcgcatctatagtggcattttaaatctgtcagacattacaaaagatacacctttctcacaaataaaagagattattattcaccaaaactataaagtctcagaagggaatcatgatatcgccttgataaaactccaggctcctttgaattacactgaattccaaaaaccaatatgcctaccttccaaaggtgacacaagcacaatttataccaactgttgggtaaccggatggggcttctcgaaggagaaaggtgaaatccaaaatattctacaaaaggtaaatattcctttggtaacaaatgaagaatgccagaaaagatatcaagattataaaataacccaacggatggtctgtgctggctataaagaagggggaaaagatgcttgtaagggagattcaggtggtcccttagtttgcaaacacaatggaatgtggcgtttggtgggcatcaccagctggggtgaaggctgtgcccgcagggagcaacctggtgtctacaccaaagtcgctgagtacatggactggattttagagaaaacacagagcagtgatggaaaagctcagatgcagtcaccagcactcgag

SEQ ID NO: 52 - protein, 619 aa, Homo sapiens
GCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTSNIRCQFFSYATQTFHKAEYRNNCLLKYSPGGTPTAIKVLSNVESGFSLKPCALSEIGCHMNIFQHLAFSDVDVARFLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNAVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 53 - DNA, 1551 bp, Homo sapiens
  CDS (108)..(1511)
gcggccgcgggagctgaccctgcggggtcccggggggggagggggagccgcgaagcccccactgaggccgccgctgccgggcctcccctcccccccgggcgggcgccatgcgggggagcccgggcgacgcggagcggcggcagcgctggggtcgcctgttcgaggagctggacagtaacaaggatggccgcgtggacgtgcacgagttgcgccaggggctggccaggctgggcgggggcaacccagaccccggcgcccaacagggtatctcctctgagggtgatgctgacccagatggcgggctcgacctggaggaattttcccgctatctgcaggagcgggaacagcgtctgctgctcatgtttcacagtcttgaccggaaccaggatggtcacattgatgtctctgagatccaacagagtttccgagctctgggcatttccatctcgctggagcaggctgagaaaattttgcacagcatggaccgagacggcacaatgaccattgactggcaagaatggcgcgaccacttcctgttgcattcgctggaaaatgtggaggacgtgctgtatttctggaagcattccacggtcctggacattggcgagtgcctgacagtgccggacgagttctcaaagcaagagaagctgacgggcatgtggtggaaacagctggtggccggcgcagtggcaggtgccgtgtcacggacaggcacggcccctctggaccgcctcaaggtcttcattcaggtccatgcctcaaagaccaaccggctgaacatccttggggggcttcgaagcatggtccttgagggaggcatccgctgcctgtggcgcggcaatggtattaatgtactcaagattgcccccgagtcagctatcaagttcatggcctatgaacaggtgaggagggccatcctggggcagcaggagacactgcatgtgcaggagcgcttcgtggctggctccctggctggtgccacagcccaaaccatcatttaccctatggaggtgctgaagacgcggctgaccttgcgccggacgggccagtataaggggctgctggactgcgccaggcgtatcctggagagggaggggccccgtgccttctaccgcggctacctccccaacgtgctgggcatcatcccctatgcgggcatcgacctggccgtctacgaggtcctgaagaactggtggcttcagcagtacagccacgactcggcagacccaggcatcctcgtgctcctggcctgcggtaccatatccagcacctgcggccagatagccagttacccgctggccctggtccggacccgcatgcaggcacaagcctccatcgagggtggcccccagctgtccatgctgggtctgctacgtcacatcctgtcccaggagggcatgcggggcctctaccgggggatcgcccccaacttcatgaaggttattccagctgtgagcatctcctatgtggtctacgagaacatgaagcaggccttgggggtcacgtccaggtgagggacccggagcccgtccccccaatccctcacccccc

SEQ ID NO: 54 - protein, 468 aa, Homo sapiens
MRGSPGDAERRQRWGRLFEELDSNKDGRVDVHELRQGLARLGGGNPDPGAQQGISSEGDADPDGGLDLEEFSRYLQEREQRLLLMFHSLDRNQDGHIDVSEIQQSFRALGISISLEQAEKILHSMDRDGTMTIDWQEWRDHFLLHSLENVEDVLYFWKHSTVLDIGECLTVPDEFSKQEKLTGMWWKQLVAGAVAGAVSRTGTAPLDRLKVFIQVHASKTNRLNILGGLRSMVLEGGIRCLWRGNGINVLKIAPESAIKFMAYEQVRRAILGQQETLHVQERFVAGSLAGATAQTIIYPMEVLKTRLTLRRTGQYKGLLDCARRILEREGPRAFYRGYLPNVLGIIPYAGIDLAVYEVLKNWWLQQYSHDSADPGILVLLACGTISSTCGQIASYPLALVRTRMQAQASIEGGPQLSMLGLLRHILSQEGMRGLYRGIAPNFMKVIPAVSISYVVYENMKQALGVTSR

SEQ ID NO: 55 - DNA, 953 bp, Homo sapiens
  CDS (50)..(853)
cagaggcagcatgagctgagagggtgataggaaggcggcgctagacagcatggaggactttctgctctccaatgggtaccagctgggcaagaccattggggaagggacctactcaaaagtcaaagaagcattttccaaaaaacaccaaagaaaagtggcaattaaagttatagacaagatgggaacttcctcagagtttatccagagattcctccctcgggagctccaaatcgtccgtaccctggaccacaagaacatcatccaggtgtatgagatgctggagtctgccgacgggaaaatctgcctggtgatggagctcgctgagggaggggatgtctttgactgcgtgctgaatggggggccactgcctgaaagccgggccaaggccctcttccgtcagatggttgaggccatccgctactgccatggctgtggtgtggcccaccgggacctcaaatgtgagaacgccttgttgcagggcttcaacctgaagctgactgactttggctttgccaaggtgttgcccaagtcacaccgggagctgagccagaccttctgcggcagtacagcctatgctgcccccgaggtgctgcagggcattccccacgatagcaaaaaaggtgatgtctggagcatgggtgtggtcctgtatgtcatgctctgtgccagcctaccttttgacgacacagacatccccaagatgctgtggcagcagcagaagggggtgtccttccccactcatctgagcatctcggccgattgccaggacctgctcaagaggctcctggaacccgatatgatcctccggccttcaattgaagaagttagttggcatccatggctagcaagcacttgataaaagcaatggcaagtgctctccaataaagtagggggagaaagcaaacccaaaaacccgcttctaaaatggtgatatatattttacgctttaagtt

SEQ ID NO: 56 - protein, 268 aa, Homo sapiens
MEDFLLSNGYQLGKTIGEGTYSKVKEAFSKKHQRKVAIKVIDKMGTSSEFIQRFLPRELQIVRTLDHKNIIQVYEMLESADGKICLVMELAEGGDVFDCVLNGGPLPESRAKALFRQMVEAIRYCHGCGVAHRDLKCENALLQGFNLKLTDFGFAKVLPKSHRELSQTFCGSTAYAAPEVLQGIPHDSKKGDVWSMGVVLYVMLCASLPFDDTDIPKMLWQQQKGVSFPTHLSISADCQDLLKRLLEPDMILRPSIEEVSWHPWLAST

SEQ ID NO: 57 - DNA, 2490 bp, Homo sapiens
  CDS (40)..(2457)
cgcgcgctgcctgaggacgccgcggcccccgcccccgccatgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaacggcagcaaggtgggcccggacggcacaccctacgttaccgtgctcaagacggcgggcgctaacaccaccgacaaggagctagaggttctctccttgcacaacgtcacctttgaggacgccggggagtacacctgcctggcgggcaattctattgggttttctcatcactctgcgtggctggtggtgctgccagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtgaagggccactggtccccaacaatgtgagggg

SEQ ID NO: 58 - protein, 806 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKTAGANTTDKELEVLSLHNVTFEDAGEYTCLAGNSIGFSHHSAWLVVLPAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 59 - DNA, 2427 bp, Homo sapiens
  CDS (1)..(2424)
atgggcgcccctgcctgcgccctcgcgctctgcgtggccgtggccatcgtggccggcgcctcctcggagtccttggggacggagcagcgcgtcgtggggcgagcggcagaagtcccgggcccagagcccggccagcaggagcagttggtcttcggcagcggggatgctgtggagctgagctgtcccccgcccgggggtggtcccatggggcccactgtctgggtcaaggatggcacagggctggtgccctcggagcgtgtcctggtggggccccagcggctgcaggtgctgaatgcctcccacgaggactccggggcctacagctgccggcagcggctcacgcagcgcgtactgtgccacttcagtgtgcgggtgacagacgctccatcctcgggagatgacgaagacggggaggacgaggctgaggacacaggtgtggacacaggggccccttactggacacggcccgagcggatggacaagaagctgctggccgtgccggccgccaacaccgtccgcttccgctgcccagccgctggcaaccccactccctccatctcctggctgaagaacggcagggagttccgcggcgagcaccgcattggaggcatcaagctgcggcatcagcagtggagcctggtcatggaaagcgtggtgccctcggaccgcggcaactacacctgcgtcgtggagaacaagtttggcagcatccggcagacgtacacgctggacgtgctggagcgctccccgcaccggcccatcctgcaggcggggctgccggccaaccagacggcggtgctgggcagcgacgtggagttccactgcaaggtgtacagtgacgcacagccccacatccagtggctcaagcacgtggaggtgaacggcagcaaggtgggcccggacggcacaccctacgttaccgtgctcaagtcctggatcagtgagagtgtggaggccgacgtgcgcctccgcctggccaatgtgtcggagcgggacgggggcgagtacctctgtcgagccaccaatttcataggcgtggccgagaaggccttttggctgagcgttcacgggccccgagcagccgaggaggagctggtggaggctgacgaggcgggcagtgtgtatgcaggcatcctcagctacggggtgggcttcttcctgttcatcctggtggtggcggctgtgacgctctgccgcctgcgcagcccccccaagaaaggcctgggctcccccaccgtgcacaagatctcccgcttcccgctcaagcgacaggtgtccctggagtccaacgcgtccatgagctccaacacaccactggtgcgcatcgcaaggctgtcctcaggggagggccccacgctggccaatgtctccgagctcgagctgcctgccgaccccaaatgggagctgtctcgggcccggctgaccctgggcaagccccttggggagggctgcttcggccaggtggtcatggcggaggccatcggcattgacaaggaccgggccgccaagcctgtcaccgtagccgtgaagatgctgaaagacgatgccactgacaaggacctgtcggacctggtgtctgagatggagatgatgaagatgatcgggaaacacaaaaacatcatcaacctgctgggcgcctgcacgcagggcgggcccctgtacgtgctggtggagtacgcggccaagggtaacctgcgggagtttctgcgggcgcggcggcccccgggcctggactactccttcgacacctgcaagccgcccgaggagcagctcaccttcaaggacctggtgtcctgtgcctaccaggtggcccggggcatggagtacttggcctcccagaagtgcatccacagggacctggctgcccgcaatgtgctggtgaccgaggacaacgtgatgaagatcgcagacttcgggctggcccgggacgtgcacaacctcgactactacaagaagacaaccaacggccggctgcccgtgaagtggatggcgcctgaggccttgtttgaccgagtctacactcaccagagtgacgtctggtcctttggggtcctgctctgggagatcttcacgctggggggctccccgtaccccggcatccctgtggaggagctcttcaagctgctgaaggagggccaccgcatggacaagcccgccaactgcacacacgacctgtacatgatcatgcgggagtgctggcatgccgcgccctcccagaggcccaccttcaagcagctggtggaggacctggaccgtgtccttaccgtgacgtccaccgacgagtacctggacctgtcggcgcctttcgagcagtactccccgggtggccaggacacccccagctccagctcctcaggggacgactccgtgtttgcccacgacctgctgcccccggccccacccagcagtgggggctcgcggacgtga

SEQ ID NO: 60 - protein, 808 aa, Homo sapiens
MGAPACALALCVAVAIVAGASSESLGTEQRVVGRAAEVPGPEPGQQEQLVFGSGDAVELSCPPPGGGPMGPTVWVKDGTGLVPSERVLVGPQRLQVLNASHEDSGAYSCRQRLTQRVLCHFSVRVTDAPSSGDDEDGEDEAEDTGVDTGAPYWTRPERMDKKLLAVPAANTVRFRCPAAGNPTPSISWLKNGREFRGEHRIGGIKLRHQQWSLVMESVVPSDRGNYTCVVENKFGSIRQTYTLDVLERSPHRPILQAGLPANQTAVLGSDVEFHCKVYSDAQPHIQWLKHVEVNGSKVGPDGTPYVTVLKSWISESVEADVRLRLANVSERDGGEYLCRATNFIGVAEKAFWLSVHGPRAAEEELVEADEAGSVYAGILSYGVGFFLFILVVAAVTLCRLRSPPKKGLGSPTVHKISRFPLKRQVSLESNASMSSNTPLVRIARLSSGEGPTLANVSELELPADPKWELSRARLTLGKPLGEGCFGQVVMAEAIGIDKDRAAKPVTVAVKMLKDDATDKDLSDLVSEMEMMKMIGKHKNIINLLGACTQGGPLYVLVEYAAKGNLREFLRARRPPGLDYSFDTCKPPEEQLTFKDLVSCAYQVARGMEYLASQKCIHRDLAARNVLVTEDNVMKIADFGLARDVHNLDYYKKTTNGRLPVKWMAPEALFDRVYTHQSDVWSFGVLLWEIFTLGGSPYPGIPVEELFKLLKEGHRMDKPANCTHDLYMIMRECWHAAPSQRPTFKQLVEDLDRVLTVTSTDEYLDLSAPFEQYSPGGQDTPSSSSSGDDSVFAHDLLPPAPPSSGGSRT

SEQ ID NO: 61 - DNA, 2077 bp, Homo sapiens
  CDS (1)..(1977)
atggagcggtggcgcgaccggctggcgctggtgacgggggcctcggggggcatcggcgcggccgtggcccgggccctggtccagcagggactgaaggtggtgggctgcgcccgcactgtgggcaacatcgaggagctggctgctgaatgtaagagtgcaggctaccccgggactttgatcccctacagatgtgacctatcaaatgaagaggacatcctctccatgttctcagctatccgttctcagcacagcggtgtagacatctgcatcaacaatgctggcttggcccggcctgacaccctgctctcaggcagcaccagtggttggaaggacatgttcaatgtgaacgtgctggccctcagcatctgcacacgggaagcctaccagtccatgaaggagcggaatgtggacgatgggcacatcattaacatcaatagcatgtctggccaccgagtgttacccctgtctgtgacccacttctatagtgccaccaagtatgccgtcactgcgctgacagagggactgaggcaagagcttcgggaggcccagacccacatccgagccacgtggcagcttcggagggaggaggccgctgccggatatcaggcagccatcactgtgaagctggggttctgtggcctccatcctctcccctcgacctccccaagacctggcaaagctcagcccctgagaaggccctctctgttggcccagtgcatctctccaggtgtggtggagacacaattcgccttcaaactccacgacaaggaccctgagaaggcagctgccacctatgagcaaatgaagtgtctcaaacccgaggatgtggccgaggctgttatctacgtcctcagcacccccgcacacatccagattggagacatccagatgaggcccacggagcagagagctcggcggagacggctgtcgagtacccttcacctcggtgttgggagcctgggagcgaactgcggcgcgggttaccgctcccggggacgcagcaaggggcatcgagtccctggcgggagctgcgccatggcattgctctcgaccgtccggggcgcgacctggggtcgcctcgtcacccgtcatttctcccatgcagcgcggcatggggagcggcctggtggggaggagctaagccgcttgctgctggatgacctggtgccgacctctcggctggagcttctgtttggcatgaccccgtgtctcctggctctgcaggccgcccgccgctctgtggcccggctcctgctccaggcgggtaaagctgggctgcaggggaagcgggccgagctgctccggatggccgaggcgcgggacattccagttctgcggcccagacggcagaaactggacacaatgtgccgctaccaggtccaccagggtgtctgcatggaggtgagcccgctgcggccccggccttggagagaggccggggaggcgagcccaggcgacgacccccagcagttgtggctcgtcctcgatgggatccaggatccccggaattttggggctgtgctgcgttccgcacacttcctcggagtggataagaccaaagcccagcagggctggctcgtggccggcacggtgggctgcccaagcacagaggatccccagtcctccgagatccccatcatgagttgcttggagttcctctgggaacggcctactctccttgtgctggggaatgagggctcaggtctatcccaggaggtgcaggcctcctgccagcttctcctcaccatcctgccccggcgccagctgcctcctggacttgagtccttgaacgtctctgtggctgcaggaattcttcttcactccatttgcagccagaggaagggtttccccacagagggggagagaaggcagcttctccaagacccccaagaaccctcagccaggtctgaagggctcagcatggctcagcacccagggctgtcttcaggcccagagaaagagaggcaaaatgagggctgacgtggactgtccacagtgttcatgtgctggagtcagggacggccgcacctgcctccgccggctccagtgtgcggggagcctctgcctgagtgtgcac

SEQ ID NO: 62 - protein, 659 aa, Homo sapiens
MERWRDRLALVTGASGGIGAAVARALVQQGLKVVGCARTVGNIEELAAECKSAGYPGTLIPYRCDLSNEEDILSMFSAIRSQHSGVDICINNAGLARPDTLLSGSTSGWKDMFNVNVLALSICTREAYQSMKERNVDDGHIININSMSGHRVLPLSVTHFYSATKYAVTALTEGLRQELREAQTHIRATWQLRREEAAAGYQAAITVKLGFCGLHPLPSTSPRPGKAQPLRRPSLLAQCISPGVVETQFAFKLHDKDPEKAAATYEQMKCLKPEDVAEAVIYVLSTPAHIQIGDIQMRPTEQRARRRRLSSTLHLGVGSLGANCGAGYRSRGRSKGHRVPGGSCAMALLSTVRGATWGRLVTRHFSHAARHGERPGGEELSRLLLDDLVPTSRLELLFGMTPCLLALQAARRSVARLLLQAGKAGLQGKRAELLRMAEARDIPVLRPRRQKLDTMCRYQVHQGVCMEVSPLRPRPWREAGEASPGDDPQQLWLVLDGIQDPRNFGAVLRSAHFLGVDKTKAQQGWLVAGTVGCPSTEDPQSSEIPIMSCLEFLWERPTLLVLGNEGSGLSQEVQASCQLLLTILPRRQLPPGLESLNVSVAAGILLHSICSQRKGFPTEGERRQLLQDPQEPSARSEGLSMAQHPGLSSGPEKERQNEG

SEQ ID NO: 63 - DNA, 736 bp, Homo sapiens
  CDS (13)..(696)
aaagcacccgagatgaccccggctcctccaccaggagcgcggccgggcgcggcgtccctagcgggcttcgccggggtggcgtctctggggcctggggacccccgccgcgccgctgacccgcgccctctgcccccagcgctgtgcttcgccgtgagccgctcgctgctgctgacgtgcctggtgccggccgcgctgctgggcctgcgctactactacagccgcaaggtgatccgcgcctacctggagtgcgcgctgcacacggacatggcggacatcgagcagtactacatgaagccgcccggtgtgtccctgaccgccctatcccctgcaggctcctgcttctgggtggccgtgctggatggcaacgtggtgggcattgtggctgcacgggcccacgaggaggacaacacggtggagctgctgcggatgtctgtggactcacgtttccgaggcaagggcatcgccaaggcgctgggccggaaggtgctggagttcgccgtggtgcacaactactccgcggtggtgctgggcacgacggccgtcaaggtggccgcccacaagctctacgagtcgctgggcttcagacacatgggcgccagtgaccactacgtgctgccgggcatgaccctctcgctggctgagcgcctcttcttccaggtccgctaccaccgctaccgcctgcagctgcgcgaggagtgaccgccgccgctcgcccgcccgcccccccggccgccct

SEQ ID NO: 64 - protein, 228 aa, Homo sapiens
MTPAPPPGARPGAASLAGFAGVASLGPGDPRRAADPRPLPPALCFAVSRSLLLTCLVPAALLGLRYYYSRKVIRAYLECALHTDMADIEQYYMKPPGVSLTALSPAGSCFWVAVLDGNVVGIVAARAHEEDNTVELLRMSVDSRFRGKGIAKALGRKVLEFAVVHNYSAVVLGTTAVKVAAHKLYESLGFRHMGASDHYVLPGMTLSLAERLFFQVRYHRYRLQLREE

SEQ ID NO: 65 - DNA, 1748 bp, Homo sapiens
  CDS (98)..(1648)
gttcaccccaagactaagttctttcccaagttagagaagaagagagaaagcaaaaagaagagaggaaagttctcccttcccctcctccgtgcctgtcatgtcctctaagccagagccgaaggacgtccaccaactgaacgggactggcccttctgcctctccctgctcttcagatggcccagggagagagcccttggctgggacctcagagttcctggggcctgatggggctggggtagaggtggtgattgagtctcgggccaacgccaagggggttcgggaggaggacgccctgctggagaacgggagccagagcaacgaaagtgacgacgtcagcacagaccgtggccctgcgccaccttccccgctcaaggagacctccttttccatcgggctgcaagtactgtttccattcctcctggcaggctttgggaccgtggctgctggcatggtgttggacatcgtgcagcactgggaagtcttccagaaggtgacagaggtcttcatcctagtgcctgcgctgctggggctcaaagggaacctggaaatgaccctggcatcaaggctttccactgcagcgagtatcaacattggacacatggacacacccaaggagctctggcggatgatcactgggaacatggccctcatccaggtgcaggccacggtggtgggcttcctggcgtccatcgcagccgtcgtctttggctggatccctgatggccacttcagtattccgcacgccttcctgctctgtgctagcagcgtggccacagccttcattgcctccctggtactgggtatgatcatgattggagtcatcattggctctcgcaagattgggatcaacccagacaatgtggccacacccattgctgccagcctgggcgacctcatcaccttggcgctgctctcaggcatcagctggggactcctgacctctgccctctcagatcactggcgatacatctacccactggtgtgtgctttctttgtggccctgctgcctgtctgggtggtgctggcccgacgaagtccagccacaagggaggtgttgtactcgggctgggagcctgttatcattgccatggccatcagcagtgtgggaggcctcatcttggacaagactgtctcagaccccaactttgctgggatggctgtcttcacgcctgtgattaatggtgttgggggcaatctggtggcagtgcaggccagccgcatctccaccttcctgcacatgaatggaatgcccggagagaactctgagcaagctcctcgccgctgtcccagtccttgtaccaccttcttcagccctggtgtgaattctcgctcagcccgggtcctcttcctcctcgtggtcccaggacacctggtgttcctctacaccatcagctgtatgcagggcgggcacaccaccctcacactcatcttcatcatcttctatatgacagctgcactgctccaggtgctgattctcctgtacatcgcagactggatggtgcactggatgtggggccggggcctggacccggacaacttctccatcccatacttgactgctctgggggacctgcttggcactgggctcctagcactcagcttccatgttctctggctcataggggaccgagacacggatgtcggggactagcttggtcactcaacattttccccatccctctgcactttctatttgaaatttttcttttgttcccctgtccctcctccaccccacactcccacctctt

SEQ ID NO: 66 - protein, 517 aa, Homo sapiens
MSSKPEPKDVHQLNGTGPSASPCSSDGPGREPLAGTSEFLGPDGAGVEVVIESRANAKGVREEDALLENGSQSNESDDVSTDRGPAPPSPLKETSFSIGLQVLFPFLLAGFGTVAAGMVLDIVQHWEVFQKVTEVFILVPALLGLKGNLEMTLASRLSTAASINIGHMDTPKELWRMITGNMALIQVQATVVGFLASIAAVVFGWIPDGHFSIPHAFLLCASSVATAFIASLVLGMIMIGVIIGSRKIGINPDNVATPIAASLGDLITLALLSGISWGLLTSALSDHWRYIYPLVCAFFVALLPVWVVLARRSPATREVLYSGWEPVIIAMAISSVGGLILDKTVSDPNFAGMAVFTPVINGVGGNLVAVQASRISTFLHMNGMPGENSEQAPRRCPSPCTTFFSPGVNSRSARVLFLLVVPGHLVFLYTISCMQGGHTTLTLIFIIFYMTAALLQVLILLYIADWMVHWMWGRGLDPDNFSIPYLTALGDLLGTGLLALSFHVLWLIGDRDTDVGD

SEQ ID NO: 67 - DNA, 1899 bp, Homo sapiens
  CDS (31)..(1878)
cgtctcgcccgccagtctccctcccgcgcgatggcctcggcgctgagctatgtctccaagttcaagtccttcgtgatcttgttcgtcaccccgctcctgctgctgccactcgtcattctgatgcccgccaaggtcagttgctgtgcctacgtcatcatcctcatggccatttactggtgcacagaagtcatccctctggctgtcacctctctcatgcctgtcttgcttttcccactcttccagattctggactccaggcaggtgtgtgtccagtacatgaaggacaccaacatgctgttcctgggcggcctcatcgtggccgtggctgtggagcgctggaacctgcacaagaggatcgccctgcgcacgctcctctgggtgggggccaagcctgcacggctgatgctgggcttcatgggcgtcacagccctcctgtccatgtggatcagtaacacggcaaccacggccatgatggtgcccatcgtggaggccatattgcagcagatggaagccacaagcgcagccaccgaggccggcctggagggacaaggtaccacaataaacaacctgaatgcactggaggatgatacagtgaaagcagtactaggaggaaagtgtgtagctataataagcacttacgtcaaaaaagtagaaaaacttcaaataaacaatctaatgacacctcttaaaaaactagaaaagcaagagcaacaggacctagggcctggcatcaggcctcaggactctgcccagtgccaggaagaccaagagcggaagaggttgtgtaaggccatgaccctgtgcatctgctacgcggccagcatcgggggcaccgccaccctgaccgggacgggacccaacgtggtgctcctgggccagatgaacgagttgtttcctgacagcaaggacctcgtgaactttgcttcctggtttgcatttgcctttcccaacatgctggtgatgctgctgttcgcctggctgtggctccagtttgtttacatgttctccagttttaaaaagtcctggggctgcgggctagagagcaagaaaaacgagaaggctgccctcaaggtgctgcaggaggagtaccggaagctggggcccttgtccttcgcggagatcaacgtgctgatctgcttcttcctgctggtcatcctgtggttctcccgagaccccggcttcatgcccggctggctgactgttgcctgggtggagggtgagacaaagtcagtctccgatgccactgtggccatctttgtggccaccctgctattcattgtgccttcacagaagcccaagtttaacttccgcagccagactgaggaaggtaagtctcctgttctgatcgccccccctcccctgctggattggaaggtaacccaggagaaagtgccctggggcatcgtgctgctactagggggcggatttgctctggctaaaggatccgaggcctcggggctgtccgtgtggatggggaagcagatggagcccttgcacgcagtgcccccggcagccatcaccttgatcttgtccttgctcgttgccgtgttcactgagtgcacaagcaacgtggccaccaccaccttgttcctgcccatctttgcctccatgtctcgctccatcggcctcaatccgctgtacatcatgctgccctgtaccctgagtgcctcctttgccttcatgttgcctgtggccacccctccaaatgccatcgtgttcacctatgggcacctcaaggttgctgacatggtaaaaacaggagtcataatgaacataattggagtcttctgtgtgtttttggctgtcaacacctggggacgggccatatttgacttggatcatttccctgactgggctaatgtgacacatattgagacttaggaagagccacaagaccac

SEQ ID NO: 68 - protein, 616 aa, Homo sapiens [sequence omitted]

SEQ ID NO: 69 - DNA, 1790 bp, Homo sapiens
  CDS (16)..(1719)
tctccctcccgcgcgatggcctcggcgctgagctatgtctccaagttcaagtccttcgtgatcttgttcgtcaccccgctcctgctgctgccactcgtcattctgatgcccgccaaggtcagttgtgcctacgtcatcatcctcatggccatttactggtgcacagaagtcatccctctggctgtcacctctctcatgcctgtcttgcttttcccactcttccagattctggactccaggcaggtgtgtgtccagtacatgaaggacaccaacatgctgttcctgggcggcctcatcgtggccgtggctgtggagcgctggaacctgcacaagaggatcgccctgcgcacgctcctctgggtgggggccaagcctgcacggctgatgctgggcttcatgggcgtcacagccctcctgtccatgtggatcagtaacacggcaaccacggccatgatggtgcccatcgtggaggccatattgcagcagatggaagccacaagcgcagccaccgaggccggcctggagctggtggacaagggcaaggccaaggagctgccagggagtcaagtgatttttgaaggccccactctggggcagcaggaagaccaagagcggaagaggttgtgtaaggccatgaccctgtgcatctgctacgcggccagcatcgggggcaccgccaccctgaccgggacgggacccaacgtggtgctcctgggccagatgaacgagttgtttcctgacagcaaggacctcgtgaactttgcttcctggtttgcatttgcctttcccaacatgctggtgatgctgctgttcgcctggctgtggctccagtttgtttacatgttctccagttttaaaaagtcctggggctgcgggctagagagcaagaaaaacgagaaggctgccctcaaggtgctgcaggaggagtaccggaagctggggcccttgtccttcgcggagatcaacgtgctgatctgcttcttcctgctggtcatcctgtggttctcccgagaccccggcttcatgcccggctggctgactgttgcctgggtggagggtgagacaaagtatgtctccgatgccactgtggccatctttgtggccaccctgctattcattgtgccttcacagaagcccaagtttaacttccgcagccagactgaggaaggtaagtctcctgttctgatcgccccccctcccctgctggattggaaggtaacccaggagaaagtgccctggggcatcgtgctgctactagggggcggatttgctctggctaaaggatccgaggcctcggggctgtccgtgtggatggggaagcagatggagcccttgcacgcagtgcccccggcagccatcaccttgatcttgtccttgctcgttgccgtgttcactgagtgcacaagcaacgtggccaccaccaccttgttcctgcccatctttgcctccatgtctcgctccatcggcctcaatccgctgtacatcatgctgccctgtaccctgagtgcctcctttgccttcatgttgcctgtggccacccctccaaatgccatcgtgttcacctatgggcacctcaaggttgctgacatggtgaaaacaggagtcataatgaacataattggagtcttctgtgtgtttttggctgtcaacacctggggacgggccatatttgacttggatcatttccctgactgggctaatgtgacacatattgagacttaggaagagccacaagaccacacacacagcccttaccctcctcaggactaccgaaccttctggcacacctt

SEQ ID NO: 70 - protein, 568 aa, Homo sapiens
MASALSYVSKFKSFVILFVTPLLLLPLVILMPAKVSCAYVIILMAIYWCTEVIPLAVTSLMPVLLFPLFQILDSRQVCVQYMKDTNMLFLGGLIVAVAVERWNLHKRIALRTLLWVGAKPARLMLGFMGVTALLSMWISNTATTAMMVPIVEAILQQMEATSAATEAGLELVDKGKAKELPGSQVIFEGPTLGQQEDQERKRLCKAMTLCICYAASIGGTATLTGTGPNVVLLGQMNELFPDSKDLVNFASWFAFAFPNMLVMLLFAWLWLQFVYMFSSFKKSWGCGLESKKNEKAALKVLQEEYRKLGPLSFAEINVLICFFLLVILWFSRDPGFMPGWLTVAWVEGETKYVSDATVAIFVATLLFIVPSQKPKFNFRSQTEEGKSPVLIAPPPLLDWKVTQEKVPWGIVLLLGGGFALAKGSEASGLSVWMGKQMEPLHAVPPAAITLILSLLVAVFTECTSNVATTTLFLPIFASMSRSIGLNPLYIMLPCTLSASFAFMLPVATPPNAIVFTYGHLKVADMVKTGVIMNIIGVFCVFLAVNTWGRAIFDLDHFPDWANVTHIET

SEQ ID NO: 71 - DNA, 3147 bp, Homo sapiens
  CDS (2)..(1705)
gatggcctcggcgctgagctatgtctccaagttcaagtccttcgtgatcttgttcgtcaccccgctcctgctgctgccactcgtcattctgatgcccgccaagtttgtcaggtgtgcctacgtcatcatcctcatggccatttactggtgcacagaagtcatccctctggctgtcacctctctcatgcctgtcttgcttttcccactcttccagattctggactccaggcaggtgtgtgtccagtacatgaaggacaccaacatgctgttcctgggcggcctcatcgtggccgtggctgtggagcgctggaacctgcacaagaggatcgccctgcgcacgctcctctgggtgggggccaagcctgcacggctgatgctgggcttcatgggcgtcacagccctcctgtccatgtggatcagtaacacggcaaccacggccatgatggtgcccatcgtggaggccatattgcagcagatggaagccacaagcgcagccaccgaggccggcctggagctggtggacaagggcaaggccaaggagctgccagggagtcaagtgatttttgaaggccccactctggggcagcaggaagaccaagagcggaagaggttgtgtaaggccatgaccctgtgcatctgctacgcggccagcatcgggggcaccgccaccctgaccgggacgggacccaacgtggtgctcctgggccagatgaacgagttgtttcctgacagcaaggacctcgtgaactttgcttcctggtttgcatttgcctttcccaacatgctggtgatgctgctgttcgcctggctgtggctccagtttgtttacatgagattcaattttaaaaagtcctggggctgcgggctagagagcaagaaaaacgagaaggctgccctcaaggtgctgcaggaggagtaccggaagttggggcccttgtccttcgcggagatcaacgtgctgatctgcttcttcctgctggtcatcctgtggttctcccgagaccccggcttcatgcccggctggctgactgttgcctgggtggagggtgagacaaagtatgtctccgatgccactgtggccatctttgtggccaccctgctattcattgtgccttcacagaagcccaagtttaacttccgcagccagactgaggaagaaaggaaaactccattttatccccctcccctgctggattggaaggtaacccaggagaaagtgccctggggcatcgtgctgctactagggggcggatttgctctggctaaaggatccgaggcctcggggctgtccgtgtggatggggaagcagatggagcccttgcacgcagtgcccccggcagccatcaccttgatcttgtccttgctcgttgccgtgttcactgagtgcacaagcaacgtggccaccaccaccttgttcctgcccatctttgcctccatgtctcgctccatcggcctcaatccgctgtacatcatgctgccctgtaccctgagtgcctcctttgccttcatgttgcctgtggccacccctccaaatgccatcgtgttcacctatgggcacctcaaggttgctgacatggtgaaaacaggagtcataatgaacataattggagtcttctgtgtgtttttggctgtcaacacctggggacgggccatatttgacttggatcatttccctgactgggctaatgtgacacatattgagacttaggaagagccacaagaccacacacacagcccttaccctcctcaggactaccgaaccttctggcacaccttgtacagagttttggggttcacaccccaaaatgacccaacgatgtccacacaccaccaaaacccagccaatgggccacctcttcctccaagcccagatgcagagatggtcatgggcagctggagggtaggctcagaaatgaagggaacccctcagtgggctgctggacccatctttcccaagccttgccattatctctgtgagggaggccaggtagccgagggatcaggatgcaggctgctgtacccgctctgcctcaagcatcccccacacagggctctggttttcactcgcttcgtcctagatagtttaaatgggaatcggatcccctggttgagagctaagacaaccacctaccagtgcccatgtcccttccagctcaccttgagcagcctcagatcatctctgtcactctggaagggacaccccagccagggacggaatgcctggtcttgagcaacctcccactgctggagtgcgagtgggaatcagagcctcctgaagcctctgggaactcctcctgtggccaccaccaaaggatgaggaatctgagttgccaacttcaggacgacacctggcttgccacccacagtgcaccacaggccaacctacgcccttcatcacttggttctgttttaatcgactggccccctgtcccacctctccagtgagcctccttcaactccttggtcccctgttgtctgggtcaacatttgccgagacgccttggctggcaccctctggggtcccccttttctcccaggcaggtcatcttttctgggagatgcttcccctgccatccccaaatagctaggatcacactccaagtatgggcagtgatggcgctctgggggccacagtgggctatctaggccctccctcacctgaggcccagagtggacacagctgttaatttccactggctatgccacttcagagtctttcatgccagcgtttgagctcctctgggtaaaatcttccctttgttgactggccttcacagccatggctggtgacaacagaggatcgttgagattgagcagcgcttggtgatctctcagcaaacaacccctgcccgtgggccaatctacttgaagttactcggacaaagaccccaaagtggggcaacaactccagagaggctgtgggaatcttcagaagcccccctgtaagagacagacatgagagacaagcatcttctttcccccgcaagtccattttatttccttcttgtgctgctctggaagagaggcagtagcaaagagatgagctcctggatggcattttccagggcaggagaaagtatgagagcctcaggaaaccccatcaaggaccgagtatgtgtctggttcctttggtggttggcttctggc

SEQ ID NO: 72 - protein, 568 aa, Homo sapiens [sequence omitted]

SEQ ID NO: 73 - DNA, 1606 bp, Homo sapiens
  CDS (2)..(1567)
gatggcctcggcgctgagctatgtctccaagttcaagtccttcgtgatcttgttcgtcaccccgctcctgctgctgccactcgtcattctgatgcccgccaagtttgtcaggtgtgcctacgtcatcatcctcatggccatttactggtgcacagaagtcatccctctggctgtcacctctctcatgcctgtcttgcttttcccactcttccagattctggactccaggcaggtgtgtgtccagtacatgaaggacaccaacatgctgttcctgggcggcctcatcgtggccgtggctgtggagcgctggaacctgcacaagaggatcgccctgcgcacgctcctctgggtgggggccaagcctgcacggctgatgctgggcttcatgggcgtcacagccctcctgtccatgtggatcagtaacacggcaaccacggccatgatggtgcccatcgtggaggccatattgcagcagatggaagccacaagcgcagccaccgaggccggcctggagctggtggacaagggcaaggccaaggagctgccagggagtcaagtgatttttgaaggccccactctggggcagcaggaagaccaagagcggaagaggttgtgtaaggccatgaccctgtgcatctgctacgcggccagcatcgggggcaccgccaccctgaccgggacgggacccaacgtggtgctcctgggccagatgaacgagttgtttcctgacagcaaggacctcgtgaactttgcttcctggtttgcatttgcctttcccaacatgctggtgatgctgctgttcgcctggctgtggctccagtttgtttacatgagattcaattttaaaaagtcctggggctgcgggctagagagcaagaaaaacgagaaggctgccctcaaggtgctgcaggaggagtaccggaagttggggcccttgtccttcgcggagatcaacgtgctgatctgcttcttcctgctggtcatcctgtggttctcccgagaccccggcttcatgcccggctggctgactgttgcctgggtggagggtgagacaaagtatgtctccgatgccactgtggccatctttgtggccaccctgctattcattgtgccttcacagaagcccaagtttaacttccgcagccagactgaggaagaaaggaaaactccattttatccccctcccctgctggattggaaggtaacccaggagaaagtgccctggggcatcgtgctgctactagggggcggatttgctctggctaaaggatccgaggcctcggggctgtccgtgtggatggggaagcagatggagcccttgcacgcagtgcccccggcagccatcaccttgatcttgtccttgctcgttgccgtgttcactgagtgcacaagcaacgtggccaccaccaccttgttcctgcccatctttgcctccatggtgaaaacaggagtcataatgaacataattggagtcttctgtgtgtttttggctgtcaacacctggggacgggccatatttgacttggatcatttccctgactgggctaatgtgacacatattgagacttaggaagagccacaagaccacacacatagcccttaccct

SEQ ID NO: 74 - protein, 522 aa, Homo sapiens
MASALSYVSKFKSFVILFVTPLLLLPLVILMPAKFVRCAYVIILMAIYWCTEVIPLAVTSLMPVLLFPLFQILDSRQVCVQYMKDTNMLFLGGLIVAVAVERWNLHKRIALRTLLWVGAKPARLMLGFMGVTALLSMWISNTATTAMMVPIVEAILQQMEATSAATEAGLELVDKGKAKELPGSQVIFEGPTLGQQEDQERKRLCKAMTLCICYAASIGGTATLTGTGPNVVLLGQMNELFPDSKDLVNFASWFAFAFPNMLVMLLFAWLWLQFVYMRFNFKKSWGCGLESKKNEKAALKVLQEEYRKLGPLSFAEINVLICFFLLVILWFSRDPGFMPGWLTVAWVEGETKYVSDATVAIFVATLLFIVPSQKPKFNFRSQTEEERKTPFYPPPLLDWKVTQEKVPWGIVLLLGGGFALAKGSEASGLSVWMGKQMEPLHAVPPAAITLILSLLVAVFTECTSNVATTTLFLPIFASMVKTGVIMNIIGVFCVFLAVNTWGRAIFDLDHFPDWANVTHIET

SEQ ID NO: 75 - DNA, 1781 bp, Homo sapiens
  CDS (2)..(1549)
gatggcctcggcgctgagctatgtctccaagttcaagtccttcgtgatcttgttcgtcaccccgctcctgctgctgccactcgtcattctgatgcccgccaagtttgtcaggtgtgcctacgtcatcatcctcatggccatttactggtgcacagaagtcatccctctggctgtcacctctctcatgcctgtcttgcttttcccactcttccagattctggactccaggcaggtgtgtgtccagtacatgaaggacaccaacatgctgttcctgggcggcctcatcgtggccgtggctgtggagcgctggaacctgcacaagaggatcgccctgcgcacgctcctctgggtgggggccaagcctgcacggctgatgctgggcttcatgggcgtcacagccctcctgtccatgtggatcagtaacacggcaaccacggccatgatggtgcccatcgtggaggccatattgcagcagatggaagccacaagcgcagccaccgaggccggcctggagctggtggacaagggcaaggccaaggagctgccagggagtcaagtgatttttgaaggccccactctggggcagcaggaagaccaagagcggaagaggttgtgtaaggccatgaccctgtgcatctgctacgcggccagcatcgggggcaccgccaccctgaccgggacgggacccaacgtggtgctcctgggccagatgaacgagttgtttcctgacagcaaggacctcgtgaactttgcttcctggtttgcatttgcctttcccaacatgctggtgatgctgctgttcgcctggctgtggctccagtttgtttacatgagattcaattttaaaaagtcctggggctgcgggctagagagcaagaaaaacgagaaggctgccctcaaggtgctgcaggaggagtaccggaagttggggcccttgtccttcgcggagatcaacgtgctgatctgcttcttcctgctggtcatcctgtggttctcccgagaccccggcttcatgcccggctggctgactgttgcctgggtggagggtgagacaaagtatgtctccgatgccactgtggccatctttgtggccaccctgctattcattgtgccttcacagaagcccaagtttaacttccgcagccagactgaggaagaaaggaaaactccattttatccccctcccctgctggattggaaggtaacccaggagaaagtgccctggggcatcgtgctgctactagggggcggatttgctctggctaaaggatccgaggcctcggggctgtccgtgtggatggggaagcagatggagcccttgcacgcagtgcccccggcagccatcaccttgatcttgtccttgctcgttgccgtgttcactgagtgcacaagcaacgtggccaccaccaccttgttcctgcccatctttgcctccatgaatcacgtccccaagagcttctgtgttctgtacggtgatgttgcagtgctgtctttccgcagtctcgctccatcggcctcaatccgctgtacatcatgctgccctgtaccctgagtgcctcctttgccttcatgttgcctgtggccacccctccaaatgccatcgtgttcacctatgggcacctcaaggttgctgacatggtgaaaacaggagtcataatgaacataattggagtcttctgtgtgtttttggctgtcaacacctggggacgggccatatttgacttggatcatttccctgactgggctaatgtgacacatattgagacttaggaagagccaca

SEQ ID NO: 76 - protein, 516 aa, Homo sapiens
MASALSYVSKFKSFVILFVTPLLLLPLVILMPAKFVRCAYVIILMAIYWCTEVIPLAVTSLMPVLLFPLFQILDSRQVCVQYMKDTNMLFLGGLIVAVAVERWNLHKRIALRTLLWVGAKPARLMLGFMGVTALLSMWISNTATTAMMVPIVEAILQQMEATSAATEAGLELVDKGKAKELPGSQVIFEGPTLGQQEDQERKRLCKAMTLCICYAASIGGTATLTGTGPNVVLLGQMNELFPDSKDLVNFASWFAFAFPNMLVMLLFAWLWLQFVYMRFNFKKSWGCGLESKKNEKAALKVLQEEYRKLGPLSFAEINVLICFFLLVILWFSRDPGFMPGWLTVAWVEGETKYVSDATVAIFVATLLFIVPSQKPKFNFRSQTEEERKTPFYPPPLLDWKVTQEKVPWGIVLLLGGGFALAKGSEASGLSVWMGKQMEPLHAVPPAAITLILSLLVAVFTECTSNVATTTLFLPIFASMNHVPKSFCVLYGDVAVLSFRSLAPSASIRCTSCCPVP

SEQ ID NO: 77 - DNA, 972 bp, Homo sapiens
  CDS (1)..(969)
atggattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacgtgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaa

SEQ ID NO: 78 - protein, 323 aa, Homo sapiens
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 79 - DNA, 983 bp, Homo sapiens
  CDS (30)..(980)
atggattcgatatcagtgtgtgaagctgaatgatggtcacttcgtgcctgtcctgggatttggcacctatgcgcctgcagaggttactcccccaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaagtggaaaaatactatttgacacagtggatctctgtgccacgtgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctggttgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaa

SEQ ID NO: 80 - protein, 317 aa, Homo sapiens
MMVTSCLSWDLAPMRLQRLLPQVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDESGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALVALRYQLQRGVVVLAKSYNEQRIRQNVQVFEFQLTSEEMKAIDGLNRNVRYLTLDIFAGPPNYPFSDEY

SEQ ID NO: 81 - DNA, 972 bp, Homo sapiens
  CDS (1)..(969)
atggattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacgtgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagatacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaa

SEQ ID NO: 82 - protein, 323 aa, Homo sapiens
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKRYNEQRIRQNVQVFEFQLTSEEMKAIDGLNRNVRYLTLDIFAGPPNYPFSDEY

SEQ ID NO: 83 - DNA, 994 bp, Homo sapiens
  CDS (16)..(978)
gccagatctcccaccatggattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacgtgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatatctcgagggtg

SEQ ID NO: 84 - protein, 321 aa, Homo sapiens
MDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSD

SEQ ID NO: 85 - DNA, 1219 bp, Homo sapiens
  CDS (24)..(992)
tgctaaccaggccagtgacagaaatggattcgaaataccagtgtgtgaagctgaatgatggtcacttcatgcctgtcctgggatttggcacctatgcgcctgcagaggttcctaaaagtaaagctctagaggccgtcaaattggcaatagaagccgggttccaccatattgattctgcacatgtttacaataatgaggagcaggttggactggccatccgaagcaagattgcagatggcagtgtgaagagagaagacatattctacacttcaaagctttggagcaattcccatcgaccagagttggtccgaccagccttggaaaggtcactgaaaaatcttcaattggactatgttgacctctatcttattcattttccagtgtctgtaaagccaggtgaggaagtgatcccaaaagatgaaaatggaaaaatactatttgacacagtggatctctgtgccacgtgggaggccatggagaagtgtaaagatgcaggattggccaagtccatcggggtgtccaacttcaaccacaggctgctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccatcgagaagaaccatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggcgacttcagtaagctacagctaagcccatcggccggaaaagaaagacaataattttgttttttcattttgaaaaaattaaatgctctctcctaaagattcttcaccta

SEQ ID NO: 86 - protein, 323 aa, Homo sapiens
MDSKYQCVKLNDGHFMPVLGFGTYAPAEVPKSKALEAVKLAIEAGFHHIDSAHVYNNEEQVGLAIRSKIADGSVKREDIFYTSKLWSNSHRPELVRPALERSLKNLQLDYVDLYLIHFPVSVKPGEEVIPKDENGKILFDTVDLCATWEAMEKCKDAGLAKSIGVSNFNHRLLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 87 - DNA, 1001 bp, Homo sapiens
  CDS (11)..(982)
catctaggccaccatggccatggattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacgtgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaaacgcgtgatc

SEQ ID NO: 88 - protein, 324 aa, Homo sapiens
TMAMDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSD

SEQ ID NO: 89 - DNA, 1012 bp, Homo sapiens
  CDS (1)..(1011)
gccggtaccaccatgggccaccatcaccaccatcacgattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacgtgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatatctcgagggtg

SEQ ID NO: 90 - protein, 337 aa, Homo sapiens
AGTTMGHHHHHHDSKYQCVKLNDGHFMPVLGFGTYAPAEVPKSKALEATKLAIEAGFRHIDSAHLYNNEEQVGLAIRSKIADGSVKREDIFYTSKLWCNSHRPELVRPALERSLKNLQLDYVDLYLIHFPVSVKPGEEVIPKDENGKILFDTVDLCATWEAVEKCKDAGLAKSIGVSNFNRRQLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEYLEG

SEQ ID NO: 91 - DNA, 1225 bp, Homo sapiens
  CDS (24)..(992)
tgctaaccaggccagtgacagaaatggattcgaaataccagtgtgtgaagctgaatgatggtcacttcatgcctgtcctgggatttggcacctatgcgcctgcagaggttcctaaaagtaaagctctagaggccgtcaaattggcaatagaagccgggttccaccatattgattctgcacatgtttacaataatgaggagcaggttggactggccatccgaagcaagattgcagatggcagtgtgaagagagaagacatattctacacttcaaagctttggagcaattcccatcgaccagagttggtccgaccagccttggaaaggtcactgaaaaatcttcaattggactatgttgacctctatcttattcattttccagtgtctgtaaagccaggtgaggaagtgatcccaaaagatgaaaatggaaaaatactatttgacacagtggatctctgtgccacgtgggaggccatggagaagtgtaaagatgcaggattggccaagtccatcggggtgtccaacttcaaccacaggctgctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccatcgagaagaaccatgggtggacccgaactccccggtgctcttggaggacccagtcctttgtgccttggcaaaaaagcacaagcgaaccccagccctgattgccctgcgctaccagctgcagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaacatggagggcattgcatgaggtctgccagaaggccctgcgtgtggatggtgacacagaggatggctctatgctggtgactggacacatcgcctctggttaaatctctcctgcttggcgacttcagtaagctacagctaagcccatcggccggaaaagaaagacaataattttgttttttcattttgaaaaaattaaatgctctctcctaaagattcttcacctaaaaaaa

SEQ ID NO: 92 - protein, 323 aa, Homo sapiens; identical to SEQ ID NO: 86

SEQ ID NO: 93 - DNA, 996 bp, Homo sapiens
  CDS (16)..(984)
caccgcggccgcaccatggattcgaaatatcagtgtgtgaagctgaatgatggtcacttcatgcctgtcctgggatttggcacctatgcgcctgcagaggttcctaaaagtaaagctttagaggccaccaaattggcaattgaagctggcttccgccatattgattctgctcatttatacaataatgaggagcaggttggactggccatccgaagcaagattgcagatggcagtgtgaagagagaagacatattctacacttcaaagctttggtgcaattcccatcgaccagagttggtccgaccagccttggaaaggtcactgaaaaatcttcaattggattatgttgacctctaccttattcattttccagtgtctgtaaagccaggtgaggaagtgatcccaaaagatgaaaatggaaaaatactatttgacacagtggatctctgtgccacgtgggaggccgtggagaagtgtaaagatgcaggattggccaagtccatcggggtgtccaacttcaaccgcaggcagctggagatgatcctcaacaagccagggctcaagtacaagcctgtctgcaaccaggtggaatgtcatccttacttcaaccagagaaaactgctggatttctgcaagtcaaaagacattgttctggttgcctatagtgctctgggatcccaccgagaagaaccatgggtggacccgaactccccggtgctcttggaggacccagtcctttgtgccttggcaaaaaagcacaagcgaaccccagccctgattgccctgcgctaccagctacagcgtggggttgtggtcctggccaagagctacaatgagcagcgcatcagacagaacgtgcaggtgtttgaattccagttgacttcagaggagatgaaagccatagatggcctaaacagaaatgtgcgatatttgacccttgatatttttgctggcccccctaattatccattttctgatgaatattaggtcgacggc

SEQ ID NO: 94 - protein, 323 aa, Homo sapiens; identical to SEQ ID NO: 78

SEQ ID NO: 95 - DNA, 3815 bp, Homo sapiens
  CDS (263)..(1789)
cggccgcgatccccaccacaccaccagcccggccgcacggggcactgagccgggtgctgagcaccggaggccccgccgaggccgggactcagatgttgaaagttaatttgtgtaaagacttatgcacgtggtgacatgagttctgcccagtgctctgaaatcaaagtgaagaaataaatccatggaagcccaggcaaatgatgggtgtagctatgactctctgaaggacctgcagagaaacgcctcctgattttgtcttacaatggaacttaaaaagtcgcctgacggtggatggggctgggtgattgtgtttgtctccttccttactcagtttttgtgttacggatccccactagctgttggagtcctgtacatagaatggctggatgcctttggtgaaggaaaaggaaaaacagcctgggttggatccctggcaagtggagttggcttgcttgcaagtcctgtctgcagtctctgtgtctcatcttttggagcaagacctgtcacaatcttcagtggcttcatggtggctggaggcctgatgttgagcagttttgctcccaatatctactttctgtttttttcctatggcattgttgtaggtcttggatgtggtttattatacactgcaacagtgaccattacgtgccagtattttgacgatcgccgaggcctagcgcttggcctgatttcaacaggttcaagcgttggccttttcatatatgctgctctgcagaggatgctggttgagttctatggactggatggatgcttgctgattgtgggtgctttagctttaaatatattagcctgtggcagtctgatgagacccctccaatcttctgattgtcctttgcctaaaaaaatagctccagaagatctaccagataaatactccatttacaatgaaaaaggaaagaatctggaagaaaacataaacattcttgacaagagctacagtagtgaggaaaaatgcaggatcacgttagccaatggtgactggaaacaagacagcctacttcataaaaaccccacagtgacacacacaaaagagcctgaaacgtacaaaaagaaagttgcagaacagacatatttttgcaaacagcttgccaagaggaagtggcagttatataaaaactactgtggtgaaactgtggctctttttaaaaacaaagtattttcagcccttttcattgctatcttactctttgacatcggagggtttccaccttcattacttatggaagatgtagcaagaagttcaaacgtgaaagaagaagagtttattatgccacttatttccattataggcattatgacagcagttggtaaactgcttttagggatactggctgacttcaagtggattaataccttgtatctttatgttgctaccttaatcatcatgggcctagccttgtgtgcaattccatttgccaaaagctatgtcacattggcgttgctttctgggatcctagggtttcttactggtaattggtccatctttccatatgtgaccacgaagactgtgggaattgaaaaattagcccatgcctatgggatattaatgttctttgctggacttggaaatagcctaggaccaccatcgttgggttggttttatgactggacccagacctatgatattgcattttattttagtggcttctgcgtcctgctgggaggttttattctgctgctggcagccttgccctcttgggatacatgcaacaagcaactccccaagccagctccaacaactttcttgtacaaagttgcctctaatgtttagaagaatattggaagacactatttttgctattttataccatatagcaacgatattttaacagattctcaagcaaattttctagagtcaagactattttctcatagcaaaatttcacaatgactgactctgaatgaattatttttttttatatatcctattttttatgtagtgtatgcgtagcctctatctcgtatttttttctatttctcctccccacaccatcaatgggactattctgttttgctgttattcactagttcttaacattgtaaaaagtttgaccagcctcagaaggctttctctgtgtaaagaagtataatttctctgctgactccatttaatccactgcaaggcacctagagagactgctcctattttaaaagtgatgcaagcatcatgataagatatgtgtgaagcccactaggaaataaatcattctcttctctatgtttgacttgctagtaaacagaagacttcaagccagccaggaaattaaagtggcgactaaaacagccttaagaattgcagtggagcaaattggtcattttttaaaaaaatatattttaacctacagtcaccagttttcattattctatttacctcactgaagtactcgcatgttgtttggtacccactgagcaactgtttcagttcctaaggtatttgctgagatgtgggtgaactccaaatggagaagtagtcactgtagactttcttcatggttgaccactccaaccttgctcacttttgcttcttggccatccactcagctgatgtttcctggaagtgctaattttacctgtttccaaattggaaacacatttctcaatcattccgttctggcaaatgggaaacatccatttgctttgggcacagtggggatgggctgcaagttcttgcatatcctcccagtgaagcatttatttgctactatcagattttaccactatcaaatataattcaagggcagaattaaacgtgagtgtgtgtgtgtgtgtgtgtgtgtgtgctatgcatgctctaagtctgcatgggatatgggaatggaaaagggcaataagaaattaatacccttatgcagttgcatttaaccttaagaaaaatgtccttgggataaactccaatgtttaatacattgattttttttctaaagaaatgggttttaaactttggtatgcatcagaattccctatagatctttttgaaaatataggtacctgggtatcacacatagaacttttaattctgctggtgtaggctgttgcccaaacatctataattttactgagctcttcaagtgattctgataacacagcctggattgagaatttttataagattggcaatggaaaaacatttattcttttaaataataatttttttaaaacccaagaggtcaggggattttataaaccaatagccaagtgttctttaaataggaggcacccttcccattgtgccaaaatcatcttttcatttattttgaaatttgtatgattattttatacttgtatgttgcctttcttcgaaggcgcctgaagcactttataaacacaaatcctcacaatacctctgtgaggtaggtaaatagtacttttctatgtagtaaacctggaatatggagaatttcataacagttcattctacttaataatgcaataatggagctccaagttgtcttggacttctacaccacactcagacttctggaaagttttctgtacctcattctttagtccctgtcaaggttagtaaataaaataagtgacataaaaaaaaaaaaaaaactaaactacttgttgtgttgaaagttcctttttgccagttatgttcaggaaacccaataacctgaaaaagtttgactttgatgtgacatcttcatattcatcaatgctgataattgtccaaaggcatcttcactatgtctgctaaataacatccaatgtgggcgttatctgttgtctaggggatgaattttaagttacaataaaatatttttctttgttttgcatcaaaaaaaaaa

SEQ ID NO: 96 - protein, 509 aa, Homo sapiens
MELKKSPDGGWGWVIVFVSFLTQFLCYGSPLAVGVLYIEWLDAFGEGKGKTAWVGSLASGVGLLASPVCSLCVSSFGARPVTIFSGFMVAGGLMLSSFAPNIYFLFFSYGIVVGLGCGLLYTATVTITCQYFDDRRGLALGLISTGSSVGLFIYAALQRMLVEFYGLDGCLLIVGALALNILACGSLMRPLQSSDCPLPKKIAPEDLPDKYSIYNEKGKNLEENINILDKSYSSEEKCRITLANGDWKQDSLLHKNPTVTHTKEPETYKKKVAEQTYFCKQLAKRKWQLYKNYCGETVALFKNKVFSALFIAILLFDIGGFPPSLLMEDVARSSNVKEEEFIMPLISIIGIMTAVGKLLLGILADFKWINTLYLYVATLIIMGLALCAIPFAKSYVTLALLSGILGFLTGNWSIFPYVTTKTVGIEKLAHAYGILMFFAGLGNSLGPPSLGWFYDWTQTYDIAFYFSGFCVLLGGFILLLAALPSWDTCNKQLPKPAPTTFLYKVASNV

SEQ ID NO: 97 - skipped

SEQ ID NO: 98 - DNA, 22 bp, Artificial Sequence
  Description of Artifical Sequence: Primer/Probe
aattctcatcgctttcatgaag

SEQ ID NO: 99 - DNA, 26 bp, Artificial Sequence
  Description of Artifical Sequence: Primer/Probe
taagtcgttcagacccatcctcctct

SEQ ID NO: 100 - DNA, 25 bp, Artificial Sequence
  Description of Artifical Sequence: Primer/Probe
ataggagttattggcaatcttctga

SEQ ID NO: 101 - DNA, 20 bp, Artificial Sequence
  Description of Artifical Sequence: Primer/Probe
tgagcaaacgcatggacatc

SEQ ID NO: 102 - DNA, 28 bp, Artificial Sequence
  Description of Artifical Sequence: Primer/Probe
tccatccaagtcacagaaagcatcgcac

SEQ ID NO: 103 - DNA, 19 bp, Artificial Sequence
  Description of Artifical Sequence: Primer/Probe
tgggttgaggcagctgtga

SEQ ID NO: 104 - DNA, 22 bp, Artificial Sequence
  Description of Artifical Sequence: Primer/Probe
gaggctctctccagtaacatca

SEQ ID NO: 105 - DNA, 26 bp, Artificial Sequence
  Description of Artifical Sequence: Primer/Probe
tactctccttgtcctctgaggcgctc

SEQ ID NO: 106 - DNA, 22 bp, Artificial Sequence
  Description of Artifical Sequence: Primer/Probe
gcagtttggttgtttggtttac

SEQ ID NO: 107 - DNA, 22 bp, Artificial Sequence
  Description of Artifical Sequence: Primer/Probe
gggaaatgggttgtccatatat

SEQ ID NO: 108 - DNA, 26 bp, Artificial Sequence
  Description of Artifical Sequence: Primer/Probe
ttcctgcagccttataagaagtccac

SEQ ID NO: 109 - DNA, 22 bp, Artificial Sequence
  Description of Artifical Sequence: Primer/Probe
atctgaaatggccagatttagc

SEQ ID NO: 110 - DNA, 22 bp, Artificial Sequence
  Description of Artifical Sequence: Primer/Probe
ccagtgccagtaccagaatatc

SEQ ID NO: 111 - DNA, 26 bp, Artificial Sequence
  Description of Artifical Sequence: Primer/Probe
taaccagacactctcagctgcaggca

SEQ ID NO: 112 - DNA, 19 bp, Artificial Sequence
  Description of Artifical Sequence: Primer/Probe
ctctctggcctctcagcaa

SEQ ID NO: 113 - DNA, 22 bp, Artificial Sequence
  Description of Artifical Sequence: Primer/Probe
gtctgcgtgaagatcaatcatt

SEQ ID NO: 114 - DNA, 25 bp, Artificial Sequence
  Description of Artifical Sequence: Primer/Probe
taggacacggactacgaccacgaca

SEQ ID NO: 115 - DNA, 22 bp, Artificial Sequence
  Description of Artifical Sequence: Primer/Probe
cggaccgtacggagtagatact

SEQ ID NO: 116 - DNA, 21 bp, Artificial Sequence
  Description of Artifical Sequence: Primer/Probe
ctactactggtggctgcgaat

SEQ ID NO: 117 - DNA, 25 bp, Artificial Sequence
  Description of Artifical Sequence: Primer/Probe
tcagatcatgacccacttgcctgga

SEQ ID NO: 118 - DNA, 22 bp, Artificial Sequence
  Description of Artifical Sequence: Primer/Probe
actcttcagcggatgtagatca

SEQ ID NO: 119 - DNA, 22 bp, Artificial Sequence
  Description of Artifical Sequence: Primer/Probe
ggacttctgtggaccttatgtg

SEQ ID NO: 120 - DNA, 26 bp, Artificial Sequence
  Description of Artifical Sequence: Primer/Probe
tttttcctctttgctggagtgctcct

SEQ ID NO: 121 - DNA, 22 bp, Artificial Sequence
  Description of Artifical Sequence: Primer/Probe
ttcctttggtttctggaacttt

SEQ ID NO: 122 - DNA, 22 bp, Artificial Sequence
  Description of Artifical Sequence: Primer/Probe
tcagaagggaatcatgatatcg

SEQ ID NO: 123 - DNA, 27 bp, Artificial Sequence
  Description of Artifical Sequence: Primer/Probe
tccttgataaaactccaggctcctttg

SEQ ID NO: 124 - DNA, 21 bp, Artificial Sequence
  Description of Artifical Sequence: Primer/Probe
tttggaaggtaggcatattgg

SEQ ID NO: 125 - DNA, 22 bp, Artificial Sequence
  Description of Artifical Sequence: Primer/Probe
gtcagcttctcttgctttgaga

SEQ ID NO: 126 - DNA, 23 bp, Artificial Sequence
  Description of Artifical Sequence: Primer/Probe
tcactgtcaggcactcgccaatg

SEQ ID NO: 127 - DNA, 22 bp, Artificial Sequence
  Description of Artifical Sequence: Primer/Probe
ctgtatttctggaagcattcca

SEQ ID NO: 128 - DNA, 22 bp, Artificial Sequence
  Description of Artifical Sequence: Primer/Probe
ttgatgtctctgagatccaaca

SEQ ID NO: 129 - DNA, 26 bp, Artificial Sequence
  Description of Artifical Sequence: Primer/Probe
tagtttccgagctctgggcatttcca

SEQ ID NO: 130 - DNA, 21 bp, Artificial Sequence
  Description of Artifical Sequence: Primer/Probe
catgctgtgcaaaattttctc

SEQ ID NO: 131 - DNA, 21 bp, Artificial Sequence
  Description of Artifical Sequence: Primer/Probe
aaaaggtgatgtctggagcat

SEQ ID NO: 132 - DNA, 26 bp, Artificial Sequence
  Description of Artifical Sequence: Primer/Probe
ttgtatgtcatgctctgtgccagcct

SEQ ID NO: 133 - DNA, 21 bp, Artificial Sequence
  Description of Artifical Sequence: Primer/Probe
gatgtctgtgtcgtcaaaagg

SEQ ID NO: 134 - DNA, 21 bp, Artificial Sequence
  Description of Artifical Sequence: Primer/Probe
aaaaggtgatgtctggagcat

SEQ ID NO: 135 - DNA, 26 bp, Artificial Sequence
  Description of Artifical Sequence: Primer/Probe
ttgtatgtcatgctctgtgccagcct

SEQ ID NO: 136 - DNA, 21 bp, Artificial Sequence
  Description of Artifical Sequence: Primer/Probe
gatgtctgtgtcgtcaaaagg

SEQ ID NO: 137 - DNA, 21 bp, Artificial Sequence
  Description of Artifical Sequence: Primer/Probe
aaaaggtgatgtctggagcat

SEQ ID NO: 138 - DNA, 26 bp, Artificial Sequence
  Description of Artifical Sequence: Primer/Probe
ttgtatgtcatgctctgtgccagcct

SEQ ID NO: 139 - DNA, 21 bp, Artificial Sequence
  Description of Artifical Sequence: Primer/Probe
gatgtctgtgtcgtcaaaagg

SEQ ID NO: 140 - DNA, 20 bp, Artificial Sequence
  Description of Artifical Sequence: Primer/Probe
cctacgttaccgtgctcaag

SEQ ID NO: 141 - DNA, 26 bp, Artificial Sequence
  Description of Artifical Sequence: Primer/Probe
tcgctaacaccaccgacaaggagcta

SEQ ID NO: 142 - DNA, 21 bp, Artificial Sequence
  Description of Artifical Sequence: Primer/Probe
gacgttgtgcaaggagagaac

SEQ ID NO: 143 - DNA, 20 bp, Artificial Sequence
  Description of Artifical Sequence: Primer/Probe
cctacgttaccgtgctcaag

SEQ ID NO: 144 - DNA, 26 bp, Artificial Sequence
  Description of Artifical Sequence: Primer/Probe
tcgctaacaccaccgacaaggagcta

SEQ ID NO: 145 - DNA, 21 bp, Artificial Sequence
  Description of Artifical Sequence: Primer/Probe
gacgttgtgcaaggagagaac

SEQ ID NO: 146 - DNA, 19 bp, Artificial Sequence
  Description of Artifical Sequence: Primer/Probe
tcaccgtagccgtgaagat

SEQ ID NO: 147 - DNA, 26 bp, Artificial Sequence
  Description of Artifical Sequence: Primer/Probe
taaagacgatgccactgacaaggacc

SEQ ID NO: 148 - DNA, 22 bp, Artificial Sequence
  Description of Artifical Sequence: Primer/Probe
ttcatcatctccatctcagaca

SEQ ID NO: 149 - DNA, 22 bp, Artificial Sequence
  Description of Artifical Sequence: Primer/Probe
ggactttgatcccctacagatg

SEQ ID NO: 150 - DNA, 26 bp, Artificial Sequence
  Description of Artifical Sequence: Primer/Probe
ttcaaatgaagaggacatcctctcca

SEQ ID NO: 151 - DNA, 22 bp, Artificial Sequence
  Description of Artifical Sequence: Primer/Probe
ctgagaacggatagctgagaac

SEQ ID NO: 152 - DNA, 20 bp, Artificial Sequence
  Description of Artifical Sequence: Primer/Probe
cgctactactacagccgcaa

SEQ ID NO: 153 - DNA, 23 bp, Artificial Sequence
  Description of Artifical Sequence: Primer/Probe
tgtgatccgcgcctacctggagt

SEQ ID NO: 154 - DNA, 20 bp, Artificial Sequence
  Description of Artifical Sequence: Primer/Probe
gggcggcttcatgtagtact

SEQ ID NO: 155 - DNA, 20 bp, Artificial Sequence
  Description of Artifical Sequence: Primer/Probe
cgctactactacagccgcaa

SEQ ID NO: 156 - DNA, 23 bp, Artificial Sequence
  Description of Artifical Sequence: Primer/Probe
tgtgatccgcgcctacctggagt

SEQ ID NO: 157 - DNA, 20 bp, Artificial Sequence
  Description of Artifical Sequence: Primer/Probe
gggcggcttcatgtagtact

SEQ ID NO: 158 - DNA, 22 bp, Artificial Sequence
  Description of Artifical Sequence: Primer/Probe
tctacaccatcagctgtatgca

SEQ ID NO: 159 - DNA, 26 bp, Artificial Sequence
  Description of Artifical Sequence: Primer/Probe
tcaccaccctcacactcatcttcatc

SEQ ID NO: 160 - DNA, 22 bp, Artificial Sequence
  Description of Artifical Sequence: Primer/Probe
gcagtgcagctgtcatatagaa

SEQ ID NO: 161 - DNA, 22 bp, Artificial Sequence
  Description of Artifical Sequence: Primer/Probe
ccatttactggtgcacagaagt

SEQ ID NO: 162 - DNA, 26 bp, Artificial Sequence
  Description of Artifical Sequence: Primer/Probe
tatccctctggctgtcacctctctca

SEQ ID NO: 163 - DNA, 22 bp, Artificial Sequence
  Description of Artifical Sequence: Primer/Probe
ggagtccagaatctggaagagt

SEQ ID NO: 164 - DNA, 22 bp, Artificial Sequence
  Description of Artifical Sequence: Primer/Probe
ccatttactggtgcacagaagt

SEQ ID NO: 165 - DNA, 26 bp, Artificial Sequence
  Description of Artifical Sequence: Primer/Probe
tatccctctggctgtcacctctctca

SEQ ID NO: 166 - DNA, 22 bp, Artificial Sequence
  Description of Artifical Sequence: Primer/Probe
ggagtccagaatctggaagagt

SEQ ID NO: 167 - skipped

SEQ ID NO: 168 - DNA, 20 bp, Artificial Sequence
  Description of Artifical Sequence: Primer/Probe
ctggccaagagctacaatga

SEQ ID NO: 169 - DNA, 26 bp, Artificial Sequence
  Description of Artifical Sequence: Primer/Probe
tcatcagacagaacgtgcaggtgttt

SEQ ID NO: 170 - DNA, 20 bp, Artificial Sequence
  Description of Artifical Sequence: Primer/Probe
aggccatctatggctttcat

SEQ ID NO: 171 - DNA, 22 bp, Artificial Sequence
  Description of Artifical Sequence: Primer/Probe
ttcctatggcattgttgtaggt

SEQ ID NO: 172 - DNA, 29 bp, Artificial Sequence
  Description of Artifical Sequence: Primer/Probe
ttggtttattatacactgcaacagtgacc

SEQ ID NO: 173 - DNA, 22 bp, Artificial Sequence
  Description of Artifical Sequence: Primer/Probe
atcgtcaaaatactggcacgta